(19) United States
(12) Patent Application Publication
(10) Pub. No.: US 2026/0347494 A1
(43) Pub. Date: Nov. 5, 2026

(54) IMMUNOTHERAPEUTIC PROTEINS

(71) Applicant: MACFARLANE BURNET INSTITUTE FOR MEDICAL RESEARCH AND PUBLIC HEALTH LIMITED, Melbourne, Victoria (AU)

(72) Inventors: Mark Phillip HOGARTH, Melbourne (AU); Bruce David WINES, Melbourne (AU); Clarissa Anne WHITEHEAD, Melbourne (AU)

(21) Appl. No.: 19/481,999

(22) PCT Filed: May 10, 2024
(86) PCT No.: PCT/AU2024/050468

§ 371 Date: Nov. 6, 2025

(30) Foreign Application Priority Data
May 10, 2023 (AU) 2023901425

(51) Int. Cl. C07K 16/28; A61P 35/00; C07K 1/107; C07K 16/32

(52) U.S. Cl. CPC C07K 16/2887; A61P 35/00; C07K 1/1077; C07K 16/2896; C07K 16/32

(57) ABSTRACT

The present disclosure relates to immunotherapeutic protein and methods of use and production thereof are disclosed, comprising one or more immunoglobulin heavy chain polypeptide comprising one or more of: a constant heavy chain domain 1 (CH1), a constant heavy chain domain 2 (CH2), a constant heavy chain domain 3 (CH3) and a hinge region, wherein the one or more polypeptide comprises a hexamerising mutation, and wherein the one or more polypeptide comprises at least one C1_{q} binding modification.

### TECHNICAL FIELD

The present disclosure relates to immunotherapeutic protein and methods of use and production thereof are disclosed, comprising one or more immunoglobulin heavy chain polypeptide comprising one or more of: a constant heavy chain domain 1 (CH1), a constant heavy chain domain 2 (CH2), a constant heavy chain domain 3 (CH3) and a hinge region, wherein the one or more polypeptide comprises a hexamerising mutation, and wherein the one or more polypeptide comprises at least one C1q binding modification.

### BACKGROUND

Human immunoglobulin is produced to recognise foreign antigens through their antigen combining regions. Such antibodies can be produced wherein the heavy chain of the immunoglobulin can be of distinct immunoglobulin classes for example human IgG, human IgA, human IgE human IgM, human IgG, human IgD.

In some cases the heavy chain of the immunoglobulin (Ig) may be composed of subclasses of an immunoglobulin class for example the antibody may comprise heavy chains of human IgG1 or human IgG2 or human IgG3 or human IgG4 which are subclasses of human IgG.

A number of effector systems can be harnessed by immunoglobulins in order to mediate an effective outcome. Such an outcome includes an immune or innate immune response to target antigens for example resistance to pathogens by antibodies induced either through infection or vaccination or monoclonal antibodies for example directed at the target for example a cancer cell or a pathogen. Another outcome is the modulation of inflammatory responses in autoimmune disease.

The heavy chains of immunoglobulins are responsible for the effector functions attributable to immunoglobulins. However not all classes or subclasses of immunoglobulins can mediate all effector functions. Indeed using the human IgG class as an example its subclasses vary in their capacity to initiate effector functions.

The two main effector systems that are harnessed by immunoglobulins are the serum complement system which is a multi-faceted cascade of serum protein activation that when activated can lead to death of targets by lysis via complement-dependent cytotoxicity (CDC) or by cellular mechanisms meditated by inflammatory leukocytes attracted to the area of complement activation or other leukocytes or tissue cells that can ingest or destroy circulating targets that have been opsonised with antibody and complement. Not all human Ig classes or subclasses are able to activate complement.

The second major system are the Fc receptors which are cell surface proteins expressed on the surface of leukocytes and other cells. These are cell surface receptors that specifically bind immunoglobulins often in the form of an immune complex (a complex of antigen with its cognate antibody). The receptors are characterised by the immunoglobulin class of the immunoglobulin they bind i.e. IgG is bound by the IgG Fc receptors—FcγR; IgA is bound by IgA Fc receptors—FcαR and so on. The interaction of the immunoglobulin with the Fc receptors can initiate a range of cellular effector responses.

Not all immunoglobulins are capable of utilising one or both effector systems. Human IgG1 or human IgG3 are universal activators of effector responses and as such are potent activators of both serum complement and cellular FcγR receptors i.e. human IgG1 or human IgG3 activate serum complement and bind to all activating type FcγR and inhibitory type FcγR.

In contrast, human IgG2 or human IgG4 subclasses are widely known to fail to activate serum complement. Furthermore they are not universal ligands of FcγR and demonstrate specific binding to limited types of FcγR i.e. IgG2 fails to bind to all but one activating-type FcγR, binding only to one allelic form of human FcγRIIa (FcγRIIa-H131) and fails to bind the inhibitory FcγRIIb and other activating-type FcγR. Human IgG4 binds only to the high affinity activating receptor FcγRI and to the inhibitory FcγRIIb.

Given the above, it is perhaps unsurprising that considerable research effort has been applied to elucidate the details of Fc interactions (e.g. with Fc γ Rs and complement proteins such as C1q and the membrane attack complex (MAC) proteins C4-C9) and identify modifications (e.g. mutations) that may be made to the Fc in order to potentially enhance the potency of therapeutic mAbs. However, very few such Fc mutations have been included to date in therapeutic mAbs; the best known being antibodies wherein the heavy chain glycan has been engineered to lack fucose and thereby result in enhanced FcγRIII interaction, for example obinutuzumab and margetuximab (which comprises six point mutations of the heavy chain of an IgG1 antibody), and others that are undergoing clinical trials but yet to be approved such as various HexaBody™ mAbs (GenMab BV; Copenhagen, Denmark) which comprise a triple mutation of the Fc fragment of IgG1, namely amino acid substitutions at positions 345 (i.e. E345R), 430 (i.e. E430G) and 440 (i.e. S440Y) ((known as “IgG-RGY”) (see International patent publication no. WO 2014/006217)), or a single mutation only at position 430 (i.e. E430G) of the Fc of IgG1 (de Jong et al., PLoS Biol 14(1):e1002344, 2016). This E430G mutation has been shown to enhance the ability of IgG1 antibodies to form hexamers on target molecules, and IgG1 mutants including the E430G mutation have been reported as displaying strongly enhanced CDC conditional on antigen binding at the target cell (de Jong et al., 2016 supra). It will therefore be apparent to those skilled in the art that there is a need in the art for modifications which enhance the therapeutic potency of immunotherapeutic proteins (e.g., mAbs).

### SUMMARY

In work leading to the present disclosure, the inventors produced a series of immunotherapeutic proteins (particularly mutant immunoglobulin G (IgG) molecules) comprising, for example, point mutations at various positions in and around the interface of the CH2 and CH3 domains of the heavy chain polypeptides. Some molecules including a mutation at position 429, considered to be a “buried” or inaccessible site within the IgG1 structure (see FIG. 1) and occupied by a histidine (His/H) residue, were found to show, for example, enhanced activation of the complement system by the classical complement pathway including binding of C1q, formation of the membrane attack complex and complement-dependent cytotoxicity in assays of complement function. In addition, the inventors obtained evidence showing that some molecules comprising an Fc region component with a mutation at position 429 are capable of forming oligomers, either in solution or upon binding to a relevant target. Further, the inventors have identified a series of C1q binding modifications that modulate binding of the immunotherapeutic protein to C1q.

Thus, in an aspect, the present invention provides an immunotherapeutic protein comprising one or more immunoglobulin heavy chain polypeptide comprising one or more of: a constant heavy chain domain 1 (CH1), a constant heavy chain domain 2 (CH2), a constant heavy chain domain 3 (CH3) and a hinge region, wherein the one or more polypeptide comprises a hexamerising mutation, and wherein the one or more polypeptide comprises at least one C1q binding modification.

Thus, in an aspect, the present invention provides an immunotherapeutic protein comprising one or more immunoglobulin heavy chain polypeptide comprising one or more of: a constant heavy chain domain 1 (CH1), a constant heavy chain domain 2 (CH2), a constant heavy chain domain 3 (CH3) and a hinge region, wherein the one or more polypeptide comprises an amino acid substitution at a position corresponding to H429 of the amino acid sequence of human IgG1, IgG2, IgG3 or IgG4 heavy chain (Eu numbering), and wherein the one or more polypeptide comprises at least one C1 q binding modification.

In an aspect, the present invention provides an oligomer comprising the immunotherapeutic protein as described herein.

In an aspect, the present invention provides a nucleic acid encoding the immunotherapeutic protein as described herein.

In an aspect, the present invention provides for the use of the immunotherapeutic protein, oligomer, or nucleic acid as described herein for treating or preventing a disease or condition in a subject, wherein the disease or condition is selected from autoimmune diseases and conditions, cardiovascular disease, neurodegenerative disease, other inflammatory diseases, transplantation condition or rejection, infectious diseases and proliferative diseases.

In an aspect, the present invention provides for the use of the immunotherapeutic protein, oligomer, or nucleic acid as described herein in the manufacture of a medicament for treating or preventing a disease or condition, wherein the disease or condition is selected from autoimmune diseases and conditions, cardiovascular disease, neurodegenerative disease, other inflammatory diseases, transplantation condition or rejection, infectious diseases and proliferative diseases.

In an aspect, the present invention provides a method for treating or preventing a disease or condition, comprising administering to the subject an effective amount of the immunotherapeutic protein, oligomer, or nucleic acid as described herein, wherein the disease or condition is selected from autoimmune diseases and conditions, cardiovascular disease, neurodegenerative disease, other inflammatory diseases, transplantation condition or rejection, infectious diseases and proliferative diseases.

In an aspect, the present invention provides a pharmaceutical composition or medicament comprising the immunotherapeutic protein, oligomer, or nucleic acid as described herein, and a pharmaceutically acceptable carrier, diluent and/or excipient.

In an aspect, the present invention provides a kit comprising at least one immunotherapeutic protein, oligomer or nucleic acid as described herein.

In an aspect, the present invention provides a method of producing an immunotherapeutic protein as described herein, comprising culturing a host cell comprising a construct encoding said protein under conditions suitable for the expression of said protein, and recovering the protein from culture supernatant under conditions of:
(i) mildly acidic pH to recover immunotherapeutic protein in a monomeric form; or
(ii) substantially neutral pH to recover immunotherapeutic protein in an oligomeric form.

In an aspect, the present invention provides a method of producing/modifying a immunotherapeutic protein comprising one or more immunoglobulin heavy chain polypeptide comprising: i) substituting the amino acid at a position corresponding to H429 of the amino acid sequence of human IgG1, IgG2, IgG3 or IgG4 heavy chain (Eu numbering) with an aromatic, cyclic or hydrophobic amino acid, and ii) introducing a C1q modification as described in Table 5 into the immunotherapeutic protein, wherein the one or more polypeptide comprises one or more of: a constant heavy chain domain 1 (CH1), a constant heavy chain domain 2 (CH2), a constant heavy chain domain 3 (CH3) and a hinge region.

In an aspect, the present invention provides a method of producing/modifying a immunotherapeutic protein comprising one or more immunoglobulin heavy chain polypeptide comprising: introducing a C1q modification as described in Table 5 into the immunotherapeutic protein, wherein the one or more polypeptide comprises one or more of: a constant heavy chain domain 1 (CH1), a constant heavy chain domain 2 (CH2), a constant heavy chain domain 3 (CH3) and a hinge region, and wherein the one or more polypeptide comprises an amino acid substitution at a position corresponding to H429 of the amino acid sequence of human IgG1, IgG2, IgG3 or IgG4 heavy chain (Eu numbering) with an aromatic, cyclic or hydrophobic amino acid.

In an aspect, the present invention provides a method of producing/modifying a immunotherapeutic protein comprising one or more immunoglobulin heavy chain polypeptide comprising: substituting the amino acid at a position corresponding to H429 of the amino acid sequence of human IgG1, IgG2, IgG3 or IgG4 heavy chain (Eu numbering) with an aromatic, cyclic or hydrophobic amino acid, wherein the one or more polypeptide comprises one or more of: a constant heavy chain domain 1 (CH1), a constant heavy chain domain 2 (CH2), a constant heavy chain domain 3 (CH3) and a hinge region, and wherein the one or more polypeptide comprises at least one C1q binding modification.

In an aspect, the present invention provides a method of enhancing complement-based lysis mediated by an immunotherapeutic protein comprising one or more immunoglobulin heavy chain polypeptide, the method comprising: i) substituting the amino acid at a position corresponding to H429 of the amino acid sequence of human IgG1, IgG2, IgG3 or IgG4 heavy chain (Eu numbering) with an aromatic, cyclic or hydrophobic amino acid, and ii) introducing a C1q modification as described in Table 5 into the immunotherapeutic protein, wherein the polypeptide comprises one or more of: a constant heavy chain domain 5 (CH1), a constant heavy chain domain 2 (CH2), a constant heavy chain domain 3 (CH3) and a hinge region.

In an aspect, the present invention provides a method of producing an immunotherapeutic protein as described herein, comprising culturing a host cell comprising a construct encoding said protein under conditions suitable for the expression of said protein, and recovering the protein from culture supernatant, e.g., using a method comprising affinity chromatography which may comprise using an elution buffer comprising a concentration of arginine of less than 130 mM and at less than or equal to pH 5.0.

In an aspect, the present invention provides a method of producing/modifying a immunotherapeutic protein comprising one or more immunoglobulin heavy chain polypeptide comprising: i) substituting an amino acid at a position corresponding to H429 of the amino acid sequence of human IgG1, IgG2, IgG3 or IgG4 heavy chain (Eu numbering) with F, Q, E, S, A, Y, L, V, G, W, R or P and ii) introducing a C1q modification as described in Table 5 into the immunotherapeutic protein, wherein the one or more polypeptide comprises one or more of: a constant heavy chain domain 1 (CH1), a constant heavy chain domain 2 (CH2), a constant heavy chain domain 3 (CH3) and a hinge region.

In an embodiment, the present invention provides a method of producing/modifying a immunotherapeutic protein comprising one or more immunoglobulin heavy chain polypeptide comprising: introducing a C1q modification as described in Table 5 into the immunotherapeutic protein, wherein the one or more polypeptide comprises one or more of: a constant heavy chain domain 1 (CH1), a constant heavy chain domain 2 (CH2), a constant heavy chain domain 3 (CH3) and a hinge region, and wherein the one or more polypeptide comprises an amino acid substation at a position corresponding to H429 of the amino acid sequence of human IgG1, IgG2, IgG3 or IgG4 heavy chain (Eu numbering) with F, Q, E, S, A, Y, L, V, G, W, R or P.

In an embodiment, the present invention provides a method of producing/modifying a immunotherapeutic protein comprising one or more immunoglobulin heavy chain polypeptide comprising: substituting an amino acid at a position corresponding to H429 of the amino acid sequence of human IgG1, IgG2, IgG3 or IgG4 heavy chain (Eu numbering) with F, Q, E, S, A, Y, L, V, G, W, R or P, wherein the one or more polypeptide comprises one or more of: a constant heavy chain domain 1 (CH1), a constant heavy chain domain 2 (CH2), a constant heavy chain domain 3 (CH3) and a hinge region, and wherein the one or more polypeptide comprises at least one C1q binding modification.

In an aspect, the present invention provides a method of enhancing complement-based lysis mediated by an immunotherapeutic protein comprising one or more immunoglobulin heavy chain polypeptide, the method comprising: i) substituting an amino acid at a position corresponding to H429 of the amino acid sequence of human IgG1, IgG2, IgG3 or IgG4 heavy chain (Eu numbering) with F, Q, E, S, A, Y, L, V, G, W, R or P, and ii) introducing a C1q modification as described in Table 5 into the immunotherapeutic protein, wherein the polypeptide comprises one or more of: a constant heavy chain domain 5 (CH1), a constant heavy chain domain 2 (CH2), a constant heavy chain domain 3 (CH3) and a hinge region.

In aspect, the present invention provides an immunotherapeutic protein comprising modified C1q binding wherein the immunotherapeutic protein comprises one or more immunoglobulin heavy chain polypeptide comprising one or more of: a constant heavy chain domain 1 (CH1), a constant heavy chain domain 2 (CH2), a constant heavy chain domain 3 (CH3) and a hinge region, wherein the one or more polypeptide comprises substitution of an amino acid at a position corresponding to H429 of the amino acid sequence of human IgG1, IgG2, IgG3 or IgG4 heavy chain (Eu numbering) with an aromatic, cyclic or hydrophobic amino acid.

In an aspect, the present invention provides a method of producing/modifying C1q binding of an immunotherapeutic protein comprising one or more immunoglobulin heavy chain polypeptide, the method comprising substituting an amino acid at a position corresponding to H429 of the amino acid sequence of human IgG1, IgG2, IgG3 or IgG4 heavy chain (Eu numbering) with an aromatic, cyclic or hydrophobic amino acid.

In an aspect, the present invention provides a method of enhancing complement-based lysis mediated by an immunotherapeutic protein comprising one or more immunoglobulin heavy chain polypeptide, the method comprising: substituting an amino acid at a position corresponding to H429 of the amino acid sequence of human IgG1, IgG2, IgG3 or IgG4 heavy chain (Eu numbering) with an aromatic, cyclic or hydrophobic amino acid.

In aspect, the present invention provides an immunotherapeutic protein comprising modified C1q binding wherein the immunotherapeutic protein comprises one or more immunoglobulin heavy chain polypeptide comprising one or more of: a constant heavy chain domain 1 (CH1), a constant heavy chain domain 2 (CH2), a constant heavy chain domain 3 (CH3) and a hinge region, wherein the one or more polypeptide comprises substitution of an amino acid at a position corresponding to H429 of the amino acid sequence of human IgG1, IgG2, IgG3 or IgG4 heavy chain (Eu numbering) with F, Q, E, S, A, Y, L, V, G, W, R or P.

In an aspect, the present invention provides a method of producing/modifying C1q binding of an immunotherapeutic protein comprising one or more immunoglobulin heavy chain polypeptide, the method comprising substituting an amino acid at a position corresponding to H429 of the amino acid sequence of human IgG1, IgG2, IgG3 or IgG4 heavy chain (Eu numbering) with F, Q, E, S, A, Y, L, V, G, W, R or P.

In an aspect, the present invention provides a method of enhancing complement-based lysis mediated by an immunotherapeutic protein comprising one or more immunoglobulin heavy chain polypeptide, the method comprising: substituting an amino acid at a position corresponding to H429 of the amino acid sequence of human IgG1, IgG2, IgG3 or IgG4 heavy chain (Eu numbering) with F, Q, E, S, A, Y, L, V, G, W, R or P.

### BRIEF DESCRIPTION OF FIGURES

FIG. 1 provides diagrammatic representations of the structure of the Fc of human IgG1 showing that the H429 residue is located in an inaccessible site within the Fc fragment. The representations are of the Human IgG1-Fc (PDB: 1Fc1) reproduced from Deisenhofer J., Biochemistry 20:2361-2370, 1981 with space filled rendering. (A) Side view of the Fc with the A-chain shown in black and B-chain in light grey with N-linked glycan shown in dark grey. The boxed area of the A-chain indicates the region enlarged in panels B and C; (B) the enlarged boxed area of panel A indicating in grey, solvent accessible amino acids of chain-B labelled according to the Eu numbering of human IgG1: methionine at position 428, (M428), glutamic acid 430 (E430), alanine 431 (A431), leucine 432 (L432), histidine H435 which sit above and obscure histidine 429 (H429) which is buried in the Fc; (C) The view of panel B after the solvent accessible residues shown in panel B are not rendered i.e. removed from view. Only now does the buried H429 shown in grey CPK, become apparent beneath these overlying neighbouring solvent accessible residues shown in panel B. Accessible surface area (ASA) for residues calculated using PISA (i.e. “Protein interfaces, surfaces and assemblies” service PISA; http://www.ebi. ac.uk/pdbe/prot_int/pistart.htm), and Krissinel E and K Henrick. J Mol Biol 372:774-797, 2007) indicates that H429 is inaccessible, recording a value of 0.0;

FIG. 2 provides representations of immunoglobulin (antibodies) and antibody-like (Ab-like) molecules showing the modular nature of the antibody:

Left panel: Provides representations of the prototypical immunoglobulin structure—Immunoglobulins are comprised of chains of variable (V) and constant (C) domains arranged in heavy (H) chains and, optionally, light (L) chains that self-assemble. Domains are further identified by chain (e.g. CH3 is the third constant domain of the heavy chain). Heavy chains (H) normally further include a distinct linking, or hinge sequence, between the Fab and Fc segments. Some H chains comprise three constant H domains, as shown in the panel (with residue H429 indicated in the CH3 domain), and others comprise four constant H domains (e.g. IgM and IgE) where CH4 is the equivalent domain to the CH3 of other antibodies (e.g. IgG and IgA). Antibodies from many species include a light (L) chain. In antibodies that lack a light chain, two H chains dimerise (H₂) and normally comprise the antibody. On the other hand, in antibodies that include a light (L) chain, the two antibody H chains will normally dimerise and a L chain is associated with each H chain (i.e. H₂L₂). Some antibodies lacking strong H chain interactions (e.g. such as human IgG4) may at times be antibody “half-molecules” comprising a monomeric H chain and one L chain (H₁L₁). The Fc fragment (or Fc) of an immunoglobulin is a dimer formed by covalent and/or non-covalent interactions between parts of each H chain (i.e. two Fc regions of the H chain, which each comprise a hinge, CH2 and CH3 domain of a heavy chain).
Right panel: Provides representations showing that the modular nature of immunoglobulins allows flexibility in the production of Ab-like molecules and fusion proteins (including immunoglobulin parts). The molecules shown all include an “H-like” chain, comprising at least a CH3 domain. In particular, the Ab-like molecules depicted each consist of a V domain for target antigen recognition joined by a linking sequence to a CH3 domain of an Fc region from, for example, an IgG (which, in variations of the molecules shown, could be an equivalent CH4 domain from an IgE or IgM). The CH3 domain can carry mutations such as those described herein at position 429 (Eu numbering). The V and CH3 domains may be linked by a sequence that may comprise heterologous linking sequences, synthetic linking sequences and/or comprise other homologous or heterologous sequences or domains linking the V and CH3 domains (e.g. as depicted in the examples shown in the figure, the V and CH3 domains can be linked by CH1-CH2 or CH2 with or without a further short polypeptide sequence such as a heterologous or synthetic linking sequence or an immunoglobulin hinge sequence), and the CH1, CH2 and hinge sequence modules may be provided in varied combinations and orders, not limited to the examples shown. The molecules shown are each in a monomeric form, which may dimerise to form homodimeric or heterodimeric forms. Similarly for the fusion proteins shown, these each consist of a target recognition moiety (depicted as “X”) joined by a linking sequence to at least a CH3 domain of an Fc region from, for example, an IgG (or an equivalent CH4 domain of an IgE or IgM). Again, the CH3 domain may include mutations such as those at position 429 (Eu numbering), and the target recognition moiety and H chain modules (e.g. the CH3 domain, CH2-CH3 or CH3-CH2 components) are linked by a sequence that may comprise heterologous or synthetic linking sequences and/or comprise other homologous or heterologous sequences or domains linking the V and CH3 domains (e.g. CH1-CH2 or CH2 with or without a further short polypeptide sequence such as a heterologous or synthetic linking sequence or an immunoglobulin hinge sequence);

FIG. 3 illustrates the conservation of the domain structure and sequence homology in immunoglobulin G (IgG) and immunoglobulin A (IgA) molecules: The sequence comparison of the hinges and constant domains of human immunoglobulin heavy (H) chain sequences are provided depicting the domain-based structure of the H chain (i.e. CH1-Hinge-CH2-CH3 defined by IgG1; Eu numbering indicated (inverted triangle) for the first amino acid of each IgG1 domain and hinge is shown thus: CH1 domain, amino acids 118 to 215; hinge amino acids 216 to 230; CH2 domain amino acids 231 to 340; and CH3 domain amino acids 341 to 447). The other IgG subclasses IgG2, IgG3, IgG4, as well as IgA subclasses, IgA1 and IgA2, have a corresponding domain structure with amino acid sequence homology. Also shown is the conservation of the histidine residue at position 429 of human IgG1 across the other IgG subclasses and IgA subclasses (indicated by the arrow). Amino acid sequences were derived from translation of open reading frames of sequences from the European Nucleotide Archive (https://www.ebi.ac.uk/ena/browser/). Accession numbers of the H chain sequences are: IgG1 H chain, J00228-IGHG1; IgG3 H chain, X03604-IGHG3; IgG4 H chain, K01316-IGHG; IgG2 H chain, J00230-IGHG2; IgAQ1 H chain, J00220-IGHA1; and IgA2 H chain, J00221-IGHA2. Alignments were performed using Clustal except for the hinges which were aligned manually;

FIG. 4 shows that the histidine of position 429 in IgG1 is conserved in all immunoglobulin classes. Comparison of the amino acid sequence of the IgG1 CH3 domain and the corresponding domains of other human immunoglobulin classes shows that Histidine 429, indicated by the arrow, is conserved in the CH3 domains of the other IgG subclasses, IgG2, IgG3 and IgG4 and the IgA subclasses IgAQ1 and IgA2, IgD and in the equivalent domain (CH4) of IgE and IgM. The IgG1 CH3 domain is defined and numbered according to the Eu numbering and the first amino acid in the CH3 domain is indicated by the inverted triangle above glycine. The amino acid sequences shown were derived from translation of open reading frames of sequences from the European Nucleotide Archive (https://www.ebi.ac.uk/ena/browser!). Accession numbers of the H chain sequences are: IgG1 CH3 domain, J00228-IGHG1; IgG3 CH3 domain, X03604-IGHG3; IgG4 CH3 domain, K01316-IGHG4; IgG2 CH3 domain, J00230-IGHG2; IgAQ1 CH3 domain, J00220-IGHA1; IgA2 CH3 domain, J00221-IGHA2; IgE CH4 domain, IGHE-CH4-J00222; IgM CH4 domain, IGHM-CH4-X57331; and IgD CH3 domain, IGHD-K02879;

FIG. 5 shows the results of the purification of an ACE2-Fc H429Y fusion protein according to the present disclosure: (A) Anion exchange (IEX) chromatography of flACE2-Fc-WT (flow through (ft), eluted fractions, and wash) with the ACE2-Fc-containing peak highlighted by *; (B) SDS-PAGE of flow through (ft) and IEX peak fractions with the flACE2-Fc H429Y migrating above the 250 kDa marker and low molecular weight (mw) impurities marked T; (C) Size-exclusion chromatography (SEC) of IEX fractions containing flACE2-Fc-WT (comparator) using a Superose 6 column, with oligomeric (oli), monomeric (mn) and low mw impurities (T) indicated; and (D) SEC of IEX fractions containing flACE2-Fc H429Y, showing the high proportion of oligomeric species. The monomeric (mn) species are considered to be single molecules (i.e. monomer molecules) comprising two copies of the respective ACE2-Fc fusion polypeptide self-associated through the Fc region components;

FIG. 6 provides graphical results showing SARS-CoV-2 RBD binding activities of ACE2-Fc fusion proteins according to the present disclosure. ACE2-Fc fusion protein binding to immobilised RBD-Ig was determined by ELISA for: (A) trACE2-Fc-WT, (B) flACE2-Fc-WT and (C) and EflACE2-Fc-WT and for the following variants thereof:
F ACE2-Fc proteins including a mutated H429F Fc region component
Yₒₗᵢ SEC purified ACE2-Fc proteins including a mutated H429Y Fc region component (oligomeric proteins)
Yₘₙ SEC purified ACE2-Fc proteins including a mutated H429Y Fc region component (monomer proteins; comprising two copies of the ACE2-Fc fusion protein dimerised through the Fc region components)
kif trACE2-Fc-WT proteins produced in the presence of the mannosidase inhibitor, kifunensine. (D) Summary of EC₅₀ binding constants for the trACE2-Fc-WT, flACE2-Fc-WT and variant proteins demonstrating a higher apparent RBD binding affinity for the flACE2-Fc-WT over trACE2-Fc-WT and a weaker binding affinity for the flACE2-Fc-H429Y monomer (Yₘₙ; i.e. comprising two copies of the flACE2-Fc-H429Y fusion protein dimerised through the Fc region components). Welch's unpaired t test p =0.0332 (*), <0.0001 (****);

FIG. 7 provides results showing that the flACE2-Fc H429Y fusion protein according to the present disclosure forms pH-dependant oligomers: SEC of flACE2-Fc H429Y purified by IEX was dialysed against (A) PBS 7.4 or (B) 100 mM citrate, 100 mM NaCl pH 5 and then SEC was performed in the same buffers. SEC at pH 5 yielded a greater proportion of monomeric (mn) fusion protein, than separation at pH 7.4. (C) Native PAGE (1 μg) of ACE2-Fc H429Y. Lane 1, SEC pH 5 oligomers (oli); Lane 2, SEC pH 5 monomer (mn); Lane 3, SEC pH 7.4 oligomers; Lane 4, SEC pH 7.4 monomer; Lane 5, SEC pH 5 monomer re-dialysed against PBS pH 7.4 and purified by SEC and the mn fractions collected as in panel D. (D) SEC chromatogram of pH 5.0 oligomers analysed in lane 1 of panel C, re-dialysed against PBS pH 7.4 and SEC performed in PBS pH 7.4. (E) Improved RBD-Ig binding activity of flACE2-Fc-H429Y monomer (Yₘₙ) prepared at pH 5. The monomers are considered to be single molecules (i.e. monomer molecules) comprising two copies of the respective ACE2-Fc fusion protein dimerised through the Fc region components;

FIG. 8 provides results showing that the SARS-CoV-2 neutralisation potency of ACE2-Fc fusion proteins according to the present disclosure is affected by the ACE2 scaffold (i.e. truncated or full-length) and Fc mutation. Neutralisation potencies of the ACE2 polypeptide and the three groups of ACE2-Fc-WT fusion and variant proteins were determined by titration to cytopathic effect (CPE) endpoint in a micro-neutralisation assay. The fusion proteins are trACE2-Fc, flACE2-Fc and EflACE2-Fc WT, and Fc variant, H429F, F; H429Y oligomers on SEC, Y_{oli l} and H429Y monomers on SEC, Yₘₙ (comprising two copies of the fusion protein dimerised through the Fc region components). The trACE2-Fc fusion proteins include the glycan-modified trACE2-Fc-kif Neutralisation endpoint mean±SEM, ANOVA with Dunnett's multiple comparisons test compared to ACE2 and ACE2-Fc WT. p=0.1234 (ns), 0.0332 (*), 0.0021 (**), 0.0002 (***), <0.0001 (****);

FIG. 9 provides the results of assays to assess the interaction of ACE2-Fc fusion proteins according to the present disclosure with FcγR. Ramos-S cells were opsonised with the ACE2-Fc WT fusion protein and variant proteins (5 μg/ml). Biotinylated (A) dimeric rsFcγRIIa or (B) dimeric rsFcγRIIIa probes, followed by streptavidin-APC were bound to the opsonised cells and binding described as median fluorescence intensity (Median FI) determined by flow cytometry. FcR binding activity of the ACE2-Fc fusion proteins was readily detected with the exception of ACE2-Fc H429Y fusion proteins which was greatly diminished (3 replicates, mean±SEM);

FIG. 10 provides the results of assays to assess the capacity of ACE2-Fc fusion proteins according to the present disclosure to mediate cell activation via FcγRIIIa. The results demonstrate that flACE2-Fc proteins are potent activators of Fc γ RIIIa with the exception of the Fc H429Y mutants in any ACE2 format which fail to stimulate. The afucosylated trACE2-Fc-kif is also a potent activator of FcγRIIIa. Ramos-S target cells were opsonised with (A) trACE2-Fc, (B) flACE2-Fc and (C) EflACE2-Fc, WT and variant proteins, including H429F, F; H429Y, oligomers, Yₒₗᵢ; H429Y monomer, Yₘₙ (comprising two copies of the fusion protein dimerised through the Fc region components); or trACE2-Fc kif produced from trACE2-Fc WT in Expi293 cells in the presence of the mannosidase inhibitor, kifunensine. In some experiments, Ramos-S target cells were separately opsonised with anti-CD20 mAb rituximab, RIT. These opsonised targets were incubated with FcγRIIIa-NF-κB-RE nanoluciferase reporter cells, and FcγRIIIa activation measured by the induction of nanoluciferase (RLU). Data was fitted to agonist response curves to estimate EC₅₀ (D) EC₅₀ (nM) values from the curve fits are shown. Mean±SEM, n≥4, ANOVA with Dunnett's multiple comparisons test comparing to trACE2-Fc WT. p=0.0021 (**), 0.0002 (***), <0.0001 (****);

FIG. 11 provides results showing that ACE2-Fc fusion proteins according to the present disclosure, comprising Fc region components with the H429F and H429Y mutations, strongly fix complement and direct complement dependent cytotoxicity killing of Ramos-S target cells; as determined using ELISA analysis of the complement fixing activity of trACE2-Fc (A, C, E) or flACE2-Fc (B, D, F) bound to SARS-CoV-2 RBD-biotin captured by plate bound avidin: C1q binding (A, B) to ACE2-Fc fusion protein variants (2.0 μg/ml) at different concentrations of avidin-captured RBD on the plate; titration of C1q (C, D) or C5b-9 (E, F) binding by serially diluted ACE2-Fc fusion protein variants bound to avidin-captured RBD-biotin (2.5 μg/ml) (mean±SEM); two independent ELISA experiments. (G) Flow cytometric analysis of complement dependent cytotoxicity (CDC) (% killing) of opsonised Ramos-S cells was determined using the presence of a 1/3 dilution of normal human serum as a source of complement (EC₅₀ (nM) values from the curve fits are shown);

FIG. 12 provides the results of assays showing that IgG1 antibodies comprising Fc region components with the H429F mutation of the CH3 domain strongly fix complement and show identical antigen binding. (A,B) TNP-BSA antigen was adsorbed at different concentrations (20 μg/ml-0.625 μg/ml) to the wells of ELISA plates and were reacted with a single concentration (2 μg/ml) of chimeric anti-TNP human IgG1 and IgG2 mAbs comprising unmodified wildtype (WT) heavy chains or with 2 μg/ml anti-TNP mAbs comprising IgG1 heavy chains comprising the Fc component mutation H429F (TNP-IgG1-H429F). The antibody-opsonised TNP-BSA coated wells were treated with (A) purified human C1q and the fixation of C1q detected with anti-C1q rabbit polyclonal antibody and (B) with human serum as a source of complement and the formation of the membrane attack complex (C5b-C9) was detected with anti-C5b-C9 rabbit polyclonal antibody. (C) provides the results from an ELISA showing the antigen binding activity of the chimeric anti-TNP mAbs used in panels A and B including the unmodified (TNP-IgG1-WT and TNP-IgG2-WT mAbs) and H429 modified mAb, namely TNP-IgG1-H429F. ELISA plates were coated with TNP-BSA then anti-TNP mAbs titrated and antibody binding detected with HRP-conjugated anti-human IgG. All mAbs showed similar antigen binding activity;

FIG. 13 provides results showing C1q fixation and MAC (C5b-9) formation by mAbs comprising IgG1 heavy chains with glutamic acid, glutamine or serine modifications at position 429 of the heavy chain. TNP-BSA (20 μg/ml) was adsorbed to the wells and reacted with chimeric human anti-TNP mAbs, titrated across the concentration range 4 μg/ml-0.125 μg/ml. The mAbs tested comprised unmodified wild type (WT) heavy chains of IgG1 (TNP-IgG1-WT) (panels A-D) or IgG2 (TNP-IgG2-WT) (panels A-C) or IgG1 H chains comprising mutations TNP-IgG1-H429Q or TNP-IgG1-H429E (A,C) and TNP-IgG1-H429S (B,D). Antibody opsonised TNP-BSA coated wells were treated with human serum as a source of complement. The fixation of C1q (A,B) was detected with anti-C1q rabbit polyclonal antibody and (C,D) the formation of the membrane attack complex (C5b-C9) was detected with rabbit anti-C5b-C9 polyclonal antibody;

FIG. 14 provides the results of flow cytometric quantitation of SEC purified mAb binding to target cell surface antigens. The binding activity of the unmodified (WT) rituximab, daratumumab-WT, 11B8-WT mAbs and their CH3-modified mutants carrying the mutations H429F or H429Y (SEC (IgG H₂L₂) peak fraction p1 and IgGₒₗᵢ, peak fraction p2) was evaluated by flow cytometry on Ramos lymphoma cells expressing CD20 and CD38. The binding activity of the unmodified trastuzumab-WT, pertuzumab-WT and their CH3 heavy chain mutants carrying the mutation H429F was evaluated on SK-OV-3 cells expressing HER2;

FIG. 15 provides elution chromotagrams of Protein A affinity purification characteristics of rituximab-WT, trastuzumab-WT and their mutants bearing modification in the CH3 of the heavy chain. The IgG was recovered from the column following elution with sodium citrate buffer pH 3.0: (A) Elution profiles of anti-CD20 rituximab-based mAbs. The rituximab-WT IgG and each mutant IgG eluted as a single homogenous peak; (B) Elution profiles of anti-HER2 trastuzumab-based mAbs. The trastuzumab-WT IgG and each mutant IgG eluted as a single homogenous peak;

FIG. 16 provides size exclusion chromatography (SEC) profiles revealing that H429 mutation can alter the physical properties of IgG. Following Protein A affinity purification, the mAbs were further purified by SEC at pH 7.2: (A) The SEC profiles of the unmodified rituximab-WT (WT) and the rituximab-H429F (H429F) mutant consisted of a single homogenous IgG peak (fractions right of the vertical dashed line) with minimal oligomeric species, (fractions left of the vertical dashed line). Thus, for example, the SEC profile of unmodified rituximab-WT showed a single major species corresponding, as expected, to IgG (H₂L₂) as confirmed by SDS-PAGE analysis (FIG. 17A) where it migrated at the expected ~150 kDa mass of unreduced IgG and which resolved, after reduction in DTT, to its ~50 kDa heavy (H) chain and ~25 kDa light (L) chain species. The rituximab-H429Y mAb on the other hand comprised both non-oligomeric IgG and oligomeric IgG; (B) The SEC profiles of Protein A purified anti-HER2 trastuzumab wild-type (WT) (that is, an unmodified form of trastuzumab) and its mutants including the amino acid substitution of H429F or H429Y. A single peak of non-oligomeric IgG, to the right of the dashed vertical line, was observed for the trastuzumab-WT, and the H429F mutant mAb, but the trastuzumab-H429Y mutant IgG (H429Y) contained both non-oligomeric IgG and oligomeric IgG. The non-oligomeric IgG peak of the trastuzumab-H429Y was coincident with that of the unmodified trastuzumab-WT, or its H429F variant and the equivalent rituximab-based mAbs in panel (A). The SEC profile of the oligomeric IgG in trastuzumab-H429Y was coincident with the oligomeric IgG in rituximab-H429Y in panel (A);

FIG. 17 provides images obtained from SDS-PAGE of SEC purified mAbs. mAbs purified by SEC were analysed by SDS-PAGE in 5-15% gradient gels with or without reduction of disulphide bonds: (A) Under non-reducing conditions, the non-oligomeric IgG peak of rituximab-WT (WT IgG) and rituximab-H429F (HF IgG) migrated as a single species at the expected ~150 kDa molecular size of IgG (i.e. H₂L₂). Following reduction in DTT, the 150 kDa IgG resolved as expected into ~50 kDa heavy (H) chain and ~25 kDa light (L) chains. Prior to reduction, the non-oligomeric IgG (HY IgG (H₂L₂)) and oligomeric (HY IgG(oli)) forms of rituximab-H429Y mAb (see FIG. 16), both migrated identically as a single 150 kDa IgG i.e. H₂L₂ species. Following reduction, both forms migrated as ~50 kDa heavy (H) chain and ~25 kDa light (L) chain; WT=wild-type, HF=H429F, HY=H429Y; M=molecular-weight markers, their masses (kD) are shown on the left side; (B) Under non-reducing conditions, the IgG peak of trastuzumab-WT (WT IgG), and trastuzumab-H429F (HF IgG) migrated as a single species at the expected ~150 kD molecular size of IgG i.e. H₂L₂. Following reduction with dithiothreitol (DTT), the 150 kDa IgG resolved as expected into ~50 kDa heavy (H) chain and ~25 kDa light (L) chains. Prior to reduction, the non-oligomeric IgG (HY IgG (H₂L₂)) and oligomeric (HY IgG(oli)) forms of trastuzumab-H429Y mAb both migrated identically as a single 150 kDa IgG species under non-reducing conditions and following reduction, as the expected ~50 kDa heavy (H) chain and ~25 kDa light (L) chain; WT=wild-type, HF=H429F, HY=H429Y; M=molecular-weight markers, their masses (kD) are shown on the left side;

FIG. 18 provides the results of experimentation showing that oligomeric and non-oligomeric forms of H429Y-modified IgG antibodies show equivalent CDC potency and that their formation is pH sensitive: (A) SEC of the rituximab-H429Y at pH7.2 showing the presence of two major IgG forms (see also FIG. 16A). The non-oligomeric IgG (H₂L₂) (right of the vertical dashed line, indicated as p1) and the oligomeric form of IgG (IgG(oli)), (left of the vertical dashed line indicated as p2) were separately collected for further evaluation of complement-dependent cytotoxicity (CDC) potency; (B) The oligomeric (p2) and non-oligomeric (p1) IgG forms from panel A, and the SEC purified unmodified rituximab-WT IgG were titrated and CDC potency was determined by flow cytometry using Ramos lymphoma cells and normal human serum diluted 1/3 as the source of complement. The extent of killing of the Ramos cells opsonised with the non-oligomeric rituximab H429Y p1 (open squares), oligomeric rituximab-H429Y p2 (triangles) or unmodified rituximab-WT (filled circles) was determined. The control background CDC of complement in the absence of mAb (no mAb C′ only) is shown as a filled diamond; and (C) The formation of IgG oligomers is sensitive to pH. Protein A purified trastuzumab-H429Y was analysed by size exclusion chromatography (SEC) either at pH 7.2 (left panel) or at pH 5.0 (right panel). At pH 7.2, both oligomeric (IgG (oli)) and non-oligomeric IgG were present, however at pH 5.0, only the single peak corresponding to non-oligomeric IgG was present;

FIG. 19 provides results which show that an H429F mutation in the CH3 domain of the IgG H chain potently promotes C1q binding and complement dependent cell-mediated cytotoxicity. Flow cytometric detection of C1q binding (fluorescence intensity) to Ramos lymphoma cells opsonised with unmodified rituximab-WT (A) or with the modified rituximab-H429F mAb (B), or to SK-OV-3 ovarian cancer cells opsonised with trastuzumab-WT (C) or with the modified trastuzumab-H429F mAb (D) is depicted. The cells opsonised with the mAbs were treated with normal human serum as a source of complement and the binding of C1q was detected by staining with anti-C1q rabbit polyclonal antibody. The C1q binding to the mAb opsonised cells is shown in the un-shaded histograms and the C1q background binding control (i.e. cells treated with serum complement in the absence of mAbs), is shown in the grey-shaded histograms. The median fluorescence intensity (MFI) values for each histogram are indicated in parentheses. (E) The complement-dependent cytotoxicity (CDC; i.e. % killing detected using Zombie Green) of Ramos lymphoma cells opsonised with rituximab-WT (filled circles) or its mutated variant mAb rituximab-H429F (filled squares), was determined by flow cytometry using normal human serum as the source of complement. CDC is greatly enhanced by mutation at position 429 in the rituximab-H429F to compared rituximab-WT mAb;

FIG. 20 shows the results of Protein A affinity chromatography of Type-II anti-CD20 11B8-WT mAb and mutant mAb including an H429 substitution. The mAbs were 11B8-WT mAb (WT) produced with a wild-type human IgG1 heavy chain. The 11B8-H429F (H429F) was produced with a modified IgG1 heavy chain wherein amino acid histidine at position 429 in the CH3 domain of the Ig heavy chain was replaced with phenylalanine;

FIG. 21 shows the results of size exclusion chromatography (SEC) purification and SDS PAGE analysis of the 11B8-WT mAb and the 11B8-H429F (H429F) mutant. (A) Chromatograms of the mAbs purified by SEC at pH 7.2; monomeric non-oligomeric IgG is shown to the right of the vertical dotted line and (B) SDS-PAGE analysis (5-15% gradient gel) of the SEC-purified monomeric IgG mAbs from panel A. Prior to reduction (Non-Reduced), all SEC purified antibodies migrated at the expected ~150 kDa molecular size of IgG i.e H₂L₂. Following reduction with DTT (Reduced), all of the antibodies resolved as expected into ~50 kDa heavy (H) chain and ~25 kDa light (L) chain. WT=wild-type, HF=H429F, M=molecular-weight markers, their masses (kD) are shown on the left side;

FIG. 22 provides results showing that H429 modification of the CH3 domain confers CDC potency on a type II anti-CD20 mAb. CDC by the 11B8-WT mAb (solid circles) or CH3 mutated 11B8-H429F mAb (solid squares). Complement-dependent lysis was determined using Ramos lymphoma cells opsonised with the mAbs at the indicated concentrations and normal human serum diluted 1/3 as the source of complement. CDC (% killing) was determined by flow cytometry using Zombie Green. The control background CDC of complement in the absence of mAb (no mAb C′ only) is shown as a filled diamond. The 11B8-WT mAb failed to mediate CDC whereas the 11B8-H429F mutant mAb mediated potent complement-dependent lysis of lymphoma cells (WT=wild type);

FIG. 23 shows the results of Protein A affinity chromatography of the anti-CD38 daratumumab-WT mAb and the daratumumab-H429F mAb. Elution chromatograms showed that each of the antibodies eluted as a single coincident homogenous peak;

FIG. 24 shows the results of size exclusion chromatography (SEC) purification and SDS-PAGE analysis of the daratumumab-WT mAb as well as the mutant mAb, daratumumab-H429F. (A) Chromatograms of the SEC purified mAbs. In each case, the mAbs contained a single non-oligomeric IgG peak (fractions right of the vertical dashed line) and the absence of oligomeric species (fractions left of the vertical dashed line); (B) SDS-PAGE analysis (5-15% gradient gel) of SEC purified IgG from panel A. Prior to reduction (Non-Reduced), all of the antibodies migrated at the expected ~150 kDa molecular size of IgG (i.e. H₂L₂), and following reduction with DTT (Reduced), all of the antibodies resolved as expected into ~50 kDa heavy (H) chain and ~25 kDa light (L) chain. WT=wild-type, HF=H429F, M=molecular-weight markers, their masses (kD) are shown on the left side;

FIG. 25 provides results which show that the H429F substitution profoundly enhances complement-dependent lysis of lymphoma cells by daratumumab-WT. The graph shows the level of CDC (% killing) by the daratumumab-WT mAb (solid circles) and daratumumab-H429F (solid squares). Background lysis (solid triangle) was determined in the absence of mAb, but in the presence of only complement. CDC was determined by flow cytometry using Zombie Green. The daratumumab-H429F mAb mediated more potent complement-dependent lysis of lymphoma cells than the unmodified daratumumab-WT;

FIG. 26 provides results showing that the H429F substitution confers complement-dependent lysis of myeloma and leukaemia cells resistant to lysis by anti-CD38 mAb. CDC by complement-dependent lysis was determined using: (A) KMS-12-PE myeloma cells opsonised with the daratumumab-WT mAb (solid circles) or daratumumab-H429F mutant mAb (solid squares) at the indicated concentrations. Background lysis (solid triangle) was determined in the absence of mAb, but in the presence of only complement; and (B) SUP-15 acute lymphoblastic leukaemia (ALL) cells which were opsonised with the daratumumab-WT mAb (solid circles) and daratumumab-H429F mutant mAb (solid squares) at the indicated concentrations. Background lysis (solid triangle) was determined in the absence of mAb but in the presence of only complement. The CDC (% killing) was determined by flow cytometry using Zombie Green;

FIG. 27 shows the results of Protein A affinity chromatography of the anti-HER2 mAb pertuzumab-WT and CH3 H429F variant. Elution chromatograms showed that each of the antibodies eluted as a single coincident homogenous peak;

FIG. 28 shows the results of size exclusion chromatography (SEC) purification and SDS-PAGE analysis of the anti-HER2 mAb pertuzumab-WT and CH3 H429F variant. (A) Chromatograms of SEC purified mAbs. Following Protein A affinity chromatography, the mAbs were further purified by size exclusion chromatography (SEC) at pH 7.2. The chromatographic profiles are shown for unmodified pertuzumab-WT and for an Fc modified variant, pertuzumab-H429F (H429F). In each case, the Protein A-purified mAbs contained a single non-oligomeric IgG peak (fractions right of the vertical dashed line) and the absence of oligomeric species (fractions left of the vertical dashed line). (B) SDS-PAGE analysis (5-15% gradient gel) of SEC purified IgG from panel A. Prior to reduction (Non-Reduced), all of the SEC purified antibodies migrated at the expected ~150 kDa molecular size of IgG (i.e. H₂L₂). Following reduction with DTT (Reduced), all of the antibodies resolved as expected into ~50 kDa heavy (H) chain and ~25 kDa light (L) chain. WT=wild-type, HF=H429F, M=molecular-weight markers shown in kilodaltons (kD);

FIG. 29 provides results showing the co-operation and functional synergy in mixtures of mAbs including amino acid substitution at position H429; in particular, the results show that H429F-modified mAbs can combine to enhance C1q binding to target cells: (A) Flow cytometric histograms of C1q binding to HER2 expressing SK-OV-3 cells opsonised (unfilled histograms) with trastuzumab-H429F alone, pertuzumab-H429F alone or a 1:1 mixture of trastuzumab-H429F and pertuzumab-H429F. Filled histograms show the background binding of C1q to non-opsonised cells. The Median Fluorescence Intensity (MFI) values for each histogram is also shown. C1q binding was detected using an anti-C1q-specific polyclonal rabbit antibody; (B) Titration of enhanced functional cooperation. SK-OV-3 cells were opsonised with titrated individual anti-HER2 mAbs, trastuzumab-WT, pertuzumab-WT, trastuzumab-H429F, pertuzumab-H429F or with 1:1 mixtures of trastuzumab-WT with pertuzumab-WT or trastuzumab-H429F with pertuzumab-H429F. The double-headed vertical arrow exemplifies extensive cooperation in the mixture of trastuzumab-H429F with pertuzumab-H429F where antibody concentrations are limiting compared to C1q binding of either mAb alone;

FIG. 30 provides graphical results showing that the co-operation and functional synergy of H429-modified mAbs enhances complement dependent killing (CDC) of target cells. The indicated rituximab-based mAbs were titrated alone (light grey columns) on Ramos cells or titrated in the presence of a fixed concentration of the indicated anti-CD38 or anti-CD20 mAbs (dark columns): (A) rituximab-WT titrated in the presence of 0.025 μg/ml, daratumumab-WT, (B) rituximab-HF titrated in the presence 0.025 μg/ml daratumumab-H429F, (C) rituximab-HF titrated in the presence of 0.5 μg/ml, 11B8-H429F. CDC (% killing) was determined by flow cytometry using Zombie Green. The dotted horizontal line in (B, C) indicates the percentage of CDC killing obtained in the absence (0 μg/ml) of rituximab-based mAbs, but in the presence only of (B) 0.025 μg/ml daratumumab-H429F or (C) 0.5 μg/ml 11B8-H429F. The black arrows in (B, C) indicate the enhanced CDC of the mixtures of mAbs above that of the individual mAbs (i.e. the CDC of either the rituximab-H429F alone at the indicated concentrations (light columns) or of 0.025 μg/ml daratumumab-H429F or 0.5 μg/ml 11B8-H429F (dark column, 0 μg/ml rituximab));

FIG. 31 provides the results of flow cytometry analysis of CDC potency of a rituximab antibody comprising an H429F mutation on normal peripheral blood CD19⁺ B lymphocytes. The results indicate that the rituximab-H429F mAb exhibits more potent CDC killing of normal peripheral blood B cells compared to unmodified rituximab-WT. Following treatment of peripheral blood mononuclear cells with the indicated mAbs and human complement, the proportion of live or dead B cells was determined by staining with anti-CD19 to identify B cells and also with Zombie Green (ZG) to identify dead cells. The cytograms show Zombie Green median fluorescence intensity (ZG MFI) of gated CD19 B cells (CD19 median fluorescence intensity (CD19 MFI). In each cytogram, the % of dead cells (ZG positive) is shown in the upper section, Q1, of each cytogram for rituximab-WT (30.4% ZG positive), rituximab-H429F (85.6% ZG positive) and the corresponding negative control mAbs trastuzumab-WT (3.05% ZG positive) and trastuzumab-H429F (3.11% ZG positive); the % live B cells are shown in the lower section, Q2;

FIG. 32 provides results showing that the effects of modification of H429 on the function of antibodies are not restricted to IgG1 and extend to other immunoglobulin types. Here, CDC potency on Ramos lymphoma cells of rituximab antibodies formatted with wild-type (WT) or H429F (HF) modified heavy chains of (A) IgG3 or (B) IgG4 subclasses were assessed;

FIG. 33 provides results showing that potent CDC of target cells mediated by mAbs is dependent on the presence of both the monoclonal antibody and serum complement. CDC (% killing) of Ramos cells by rituximab-H429F, daratumumab-H429F or 11B8-H429F, or of SUP-15 cells by daratumumab-H429F is shown. A=percent killing in the presence of both the indicated mAb and human serum as a source of complement; B=lysis in the presence of mAb only at the same concentration as in A; and C=lysis in the presence of complement only;

FIG. 34 provides results showing that the H429F substitution confers complement-dependent lysis of leukaemia cells resistant to lysis by the unmodified anti-CD38 mAb isatuximab. CDC by complement-dependent lysis was determined using SUP-15 acute lymphoblastic leukaemia (ALL) cells which were opsonised with the isatuximab-WT mAb (dashed line with filled circles) and isatuximab-H429F mutant mAb (solid line with filled squares) at the indicated concentrations. Background lysis (unfilled circle) was determined in the absence of mAb but in the presence of only complement (C′ only, no mAb). Background lysis induced by the mAbs at 5 μg/ml in the absence of complement was determined for isatuximab-WT mAb (inverted unfilled triangle) and isatuximab-H429F mutant mAb (open unfilled square). The CDC (% killing) was determined by flow cytometry using Zombie Green;

FIG. 35 provides the results of flow cytometric quantitation of the binding to Colo205 colorectal cells of purified DR5-specific mAbs that comprised unmodified wild-type (WT) H chains of the human IgG1 or IgG2 subclasses or comprised human IgG1 or human IgG2 subclass heavy chains that had been modified by replacement of histidine 429 with phenylalanine (H429F). The panels show: (A) binding of BDR5-1WT which comprised wild-type H chains of the human IgG1 subclass or binding of the BDR5-1HF mAb which comprised IgG1 heavy chains containing the H429F modification, (B) binding of BDR5-2WT which comprised unmodified heavy chains of the human IgG2 subclass or binding of the BDR5-2HF mAb which comprised IgG2 heavy chains containing the H429F modification, (C) the binding of the TDR5-1WT mAb which comprised unmodified heavy chains of the human IgG1 subclass or the TDR5-1HF mAb which comprised IgG1 heavy chains containing the H429F modification, and (D) binding of TDR5-2WT which comprised unmodified heavy chains of the human IgG2 subclass or binding the TDR5-2HF mAb which comprised IgG2 heavy chains containing the H429F modification. The mAbs were serially 2-fold titrated and the binding activity on Colo205 cells was quantified by flow cytometry using an anti-IgG secondary reagent labelled with goat anti-hIgG Fc FITC. In all panels, the level of non-specific binding of fluorescent conjugate to cells is shown (▴ conj); MFI=Median Fluorescence Intensity;

FIG. 36 provides the results of flow cytometric quantitation of the binding to target Ramos lymphoma cells of purified DR5-specific mAbs that comprised unmodified wild-type (WT) H chains of the human IgG1 or human IgG2 subclasses or comprised human IgG1 or human IgG2 heavy chains that had been modified by replacement of histidine 429 with phenylalanine (H429F). The panels show: (A) binding of BDR5-1WT which comprised unmodified heavy chains of the human IgG1 subclass or binding of the BDR5-1HF mAb which comprised IgG1 heavy chains containing the H429F modification; (B) binding of BDR5-2WT which comprised unmodified heavy chains of the human IgG2 subclass or binding of the BDR5-2HF mAb which comprised IgG2 heavy chains containing the H429F modification; (C) binding of TDR5-1WT which comprised unmodified heavy chains of the human IgG1 subclass or binding of the TDR5-1HF mAb which comprised IgG1 heavy chains containing the H429F modification; and (D) binding of TDR5-2WT which comprised unmodified heavy chains of the human IgG2 subclass or binding of the TDR5-2HF mAb which comprised IgG2 heavy chains containing the H429F modification. The mAbs were serially 2-fold titrated and the binding to Ramos cells was quantified by flow cytometry using an anti-IgG secondary reagent labelled with goat anti-hIgG Fc FITC. In all panels, the level of non-specific binding to cells was determined using an irrelevant IgG antibody (negative IgG) and the background binding of the fluorescence anti-IgG conjugate to cells is shown (▴ conj); MFI=Median Fluorescence Intensity;

FIG. 37 provides the results of flow cytometric quantitation of the binding to target KMS-12-PE myeloma cells of purified DR5-specific mAbs that comprised unmodified wild-type (WT) H chains of the human IgG1 or human IgG2 subclasses or comprised human IgG1 or human IgG2 heavy chains that had been modified by replacement of histidine 429 with phenylalanine (H429F). The binding activity is shown for the DR5-specific mAbs, BDR5 and TDR5, which comprised unmodified, wild-type, H chains of the human IgG1 subclass (BDR5-1WT, TDR5-1WT) or human IgG2 subclass (BDR5-2WT, TDR5-2WT) or comprised Fc mutated H chains of the IgG1 subclass (BDR5-1HF, TDR5-1HF) or IgG2 subclass (BDR5-2HF, TDR5-2HF) containing the H249F mutation. KMS12-PE cells were incubated with mAbs at 5 g/ml. Binding activity was quantified by flow cytometry using an anti-IgG secondary reagent labelled with FITC goat anti-hIgG Fc. The background control of anti-IgG conjugate only binding is shown (conj.) along with the background fluorescence of cells only (cells);

FIG. 38 provides results showing the survival of Colo205 colorectal cells in the presence of purified DR5-specific mAbs comprised of unmodified wild-type H chains of the human IgG1 or human IgG2 subclasses, or comprised of Fc mutated H chains of the human IgG1 or human IgG2 subclasses containing the H429F mutation. Ten thousand cells were cultured in each well of a 96 well plate in the presence of 20 μg/ml of each of the following mAbs: TDR5-1WT mAb which comprised unmodified wild-type H chains of the human IgG1 subclass, or TDR5-1HF mAb which comprised H chains of the human IgG1 subclass containing the H429F mutation, or TDR5-2WT mAb which comprised unmodified wild-type H chains of the human IgG2 subclass, or TDR5-2HF mAb which comprised H chain of the human IgG2 subclass containing the H429F mutation. Additionally, Colo205 cells were separately cultured in the presence of a mixture of 10 μg/ml TDR5-2HF and 10 μg/ml BDR5-2HF mAbs (TDR5-2HF+BDR5-2HF) both of which comprised Fc mutated human IgG2 H chains carrying H429F mutations. Cell viability was quantitated using a CCK8 colorimetric cell viability assay where absorbance (Abs450 nm) of cell culture supernatant is a measure of cell survival and viability. Maximum cell viability was determined by the culture of cells in the absence of antibody (no ab). Survival is represented by: [Abs450 nm of mAb or control treatment determined experimentally −background Abs450 of cell culture medium], the mean Abs450 and the four replicate values are shown;

FIG. 39 provides results showing the survival of Ramos lymphoma cells in the presence of purified DR5-specific mAbs comprised of unmodified wild-type or Fc mutated H chains of the human IgG1 or IgG2 subclasses containing the H429F mutation. Ten thousand cells were cultured in each well of a 96 well plate in the presence of 20 μg/ml of each of the following mAbs: TDR5-1WT mAb which comprised unmodified wild-type H chains of the human IgG1 subclass, or TDR5-1HF mAb which comprised H chain of the human IgG1 subclass containing the H429F mutation, TDR5-2WT mAb which comprised unmodified wild-type H chains of the human IgG2 subclass, or TDR5-2HF mAb which comprised H chains of the human IgG2 subclass containing the H429F mutation. Additionally, Ramos cells were separately cultured in the presence of a mixture of 10 μg/ml TDR5-2HF and 10 μg/ml BDR5-2HF mAbs (TDR5-2HF+BDR5-2HF) both of which comprised Fc mutated human IgG2 H chains carrying H429F mutations. Cell viability was quantitated using a CCK8 colorimetric cell viability assay where absorbance (Abs450 nm) of cell culture supernatant is a measure of cell survival and viability. Maximum cell viability was determined by culture of cells in the absence of antibody (no ab). Survival is represented by: [Abs450 nm of mAb or control treatment determined experimentally −background Abs450 of cell culture medium], the mean Abs450 and the four replicate values are shown;

FIG. 40 provides results showing the survival of Colo205 colorectal cells in the presence of mixtures of purified DR5-specific mAbs wherein the mAbs comprised wild-type or Fc modified H chains of the same IgG subclass. The BDR5 and TDR5 mAbs were comprised of unmodified wild-type H chains of the human IgG1 or human IgG2 subclasses or were comprised of heavy chains of the IgG1 subclass or IgG2 subclasses containing the H429F modification. Colo205 cells, 30,000 cells per well of 96-well plate, were cultured in the presence of serial 2-fold dilutions of 1:1 mixtures of mAbs comprised of identical H chains. The starting concentration of 1 μg/ml was comprised of 0.5 μg/ml of each mAb in the mixture. Thus, the mixtures used were: BDR5-1WT mAb comprising unmodified wild-type IgG1 H chain mAb mixed with TDR5-1WT comprising unmodified wild-type IgG1 H chain (BDR5-1WT+TDR5-1WT); BDR5-2WT mAb comprising unmodified wild-type IgG2 H chain mAb mixed with TDR5-2WT comprising unmodified wild-type IgG2 H chain (BDR5-2WT+TDR5-2WT); BDR5-1HF mAb comprising IgG1 H chains containing H429F modification mixed with TDR5-1HF mAb comprising IgG1 H chains containing H429F modification (BDR5-1HF+TDR5-1HF); BDR5-2HF mAb comprising IgG2 H chains containing H429F modification mixed with TDR5-2HF mAb comprising IgG2 H chains containing H429F modification (BDR5-2HF+TDR5-2HF). Cell viability was quantitated using a CCK8 colorimetric cell viability assay where absorbance (Abs450 nm) of cell culture supernatant is a measure of cell survival and viability. Maximum cell viability was determined by the culture of cells in the absence of antibody (no ab) and maximum death determined by culture of cells with sodium dodecyl sulphate (SDS). Survival is represented by: [Abs450 nm of mAb or control treatment determined experimentally−background Abs450 of cell culture medium];

FIG. 41 provides results showing the survival of Colo205 colorectal cells in the presence of different pairwise combinations of purified DR5-specific mAbs wherein the mAbs comprised wild-type or Fc mutated H chains of different IgG subclasses. MAbs comprised H chains of human IgG1 subclass or human IgG2 subclass comprising wild-type heavy chains or comprising heavy chains modified by H429F modification. Colo205 cells, 10,000 cells per well of 96-well tissue culture plate, were cultured in the presence of 1:1 mixtures of mAbs serially diluted 2-fold from 1 g/ml. The starting concentration of 1 μg/ml was comprised of 0.5 μg/ml of each mAb in the mixture. The mixtures of wild-type mAbs used were: BDR5-2WT comprising wild-type human IgG2 H chain mixed with TDR5-1WT comprising unmodified wild-type human IgG1 H chain (BDR5-2WT+TDR5-1WT); BDR5-1WT comprising wild-type human IgG1 H chain mixed with TDR5-2WT comprising unmodified wild-type human IgG2 H chain (BDR5-1WT+TDR5-2WT); and a control mixture of BDR5-2WT comprising unmodified wild-type human IgG2 H chain mixed with TDR5-2WT comprising unmodified wild-type human IgG2 H chain (BDR5-2WT+TDR5-2WT) as observed in FIG. 40. The mixtures of DR5 mAbs comprising H429F Fc-mutated H chains were: BDR5-2HF comprising H429F Fc-mutated human IgG2 H chain mixed with TDR5-1HF comprising H429F Fc-mutated human IgG1 H chain (BDR5-2HF+TDR5-1HF); BDR5-1HF comprising H429F Fc-mutated human IgG1 H chain mixed with TDR5-2HF comprising H429F Fc-mutated human IgG2 H chain (BDR5-2HF+TDR5-2HF); positive killing control mixture of BDR5-2HF comprising H429F Fc-mutated human IgG2 H chain mixed with TDR5-2HF comprising H429F Fc-mutated IgG2 H chain (BDR5 2HF+TDR5-2HF) as observed in FIG. 38 and FIG. 40. Cell viability was quantitated using a CCK8 colorimetric cell viability assay where absorbance (Abs450 nm) of cell culture supernatant is a measure of cell survival and viability. Maximum cell viability was determined by culture of cells in the absence of antibody (no ab) and maximum death determined by culture of cells with sodium dodecyl sulphate (SDS). Survival is represented by: [Abs450 nm of mAb or control treatment determined experimentally −background Abs450 of cell culture medium];

FIG. 42 provides results showing the survival of Colo205 cells in the presence of pairwise mixtures of BDR5 and TDR5 mAbs at different ratios. Ten thousand Colo205 cells were cultured for 48 hours in the presence of individual mAbs BDR5-1WT or TDR5-1WT comprising wild-type IgG1 H chains or individual mAbs BDR5-1HF or TDR5-1HF comprising H chains with the H429F modification or mixtures of BDR5-1HF with TDR5-1HF mAbs at the following ratios, 90:10, 75:25, 50:50, 25:75, and 10:90. In the mixes shown in the plot, the BDR5-1HF mAb is designated as B-1HF and TDR5-1HF is designated as T-1HF. Controls include maximum cell survival in the absence of mAbs (no ab) or maximum death control in the presence of sodium dodecyl sulphate (SDS). Cell viability was quantitated using a CCK8 colorimetric cell viability assay where absorbance (Abs450 nm) of cell culture supernatant is a measure of cell survival and viability. Survival is represented by: [Abs450 nm of mAb or control treatment determined experimentally −background Abs450 of cell culture medium];

FIG. 43 provides results showing the survival of Ramos lymphoma cells in the presence of pairwise mixtures of BDR5 and TDR5 mAbs at different ratios. 10,000 Ramos cells were cultured for 48 hours in the presence of individual mAbs BDR5-1WT or TDR5-1WT comprising wild-type H chains or individual mAbs BDR5-1HF or TDR5-1HF comprising H chains with the H429F modification or mixtures of BDR5-1HF with TDR5-1HF mAbs at the following ratios, 90:10, 75:25, 50:50, 25:75, and 10:90. In the mixes shown in the plot, the BDR5-1HF mAb is designated as B-1HF and TDR5-1HF is designated as T-1HF. Cell viability was quantitated using a CCK8 colorimetric cell viability assay where absorbance (Abs450 nm) of cell culture supernatant is a measure of cell survival and viability. Maximum cell viability was determined by culture of cells in the absence of antibody (no ab) and maximum death determined by culture of cells with sodium dodecyl sulphate (SDS). Survival is represented by: [Abs450 nm of mAb or control treatment determined experimentally −background Abs450 of cell culture medium];

FIG. 44 provides results quantitating the binding to Colo205 cells of DR5-specific mAbs that detect distinct epitopes and comprise wild-type or Fc mutated heavy chains of the human IgA2 subclass. The mAbs, used as tissue culture supernatants from appropriately transfected Expi293 cells, comprised unmodified wild-type IgA2 heavy chain (BDR5-A2WT) and (TDR5-A2WT) or Fc mutated IgA2 H chains carrying the H429F mutation (BDR5-A2HF) and (TDR5-A2HF) of the human IgA2 subclass. Background fluorescence determined by measuring binding of fluorescence conjugate only (no ab) is shown along with the non-specific fluorescence of cells only (cells);

FIG. 45 provides results showing the Colo205 cell survival in the presence of two distinct DR5-specific mAbs comprising heavy chains of the human IgA2 subclass. Colo205 cells, 10,000 cells per well of 96-well plate, were cultured for 48 hours in the presence of serial 2-fold dilutions of tissue culture supernatants from Expi293 cells producing the DR5 IgA mAbs used in FIG. 44. The mAbs used were: BDR5-A2WT which comprised wild-type heavy chain of the human IgA2 subclass; TDR5-A2WT which comprised wild-type heavy chain of human IgA2 subclass; BDR5-A2HF which comprised heavy chains of the human IgA2 subclass carrying the H429F modification; TDR5-A2HF which comprised heavy chains of the human IgA2 subclass carrying the H429F modification. Cell viability was quantitated using a CCK8 colorimetric cell viability assay where absorbance (Abs450 nm) of cell culture supernatant is a measure of cell survival and viability. Maximum cell viability was determined by culture of cells in the absence of antibody (no ab) and maximum death determined by culture of cells with sodium dodecyl sulphate (SDS). Survival is represented by: [experimentally determined Abs450 nm −background Abs450 of cell culture medium];

FIG. 46 provides results showing the enhanced killing of Ramos lymphoma cells by mixtures of H429F-modified mAbs detecting distinct molecular targets. Ramos cells at 10,000 per well were separately cultured in the presence of anti-CD38 mAb isatuximab comprising IgG1 WT H Chains (Isa-WT), or anti-DR5 mAb BDR5-1 comprising IgG1 WT H chains (BDR5-1WT), or a 1:1 mixture of both mAbs (Isa-WT+BDR5-1WT) or cultured with their Fc modified counterparts, isatuximab comprising H chains carrying the H429F modification (Isa-HF) as described in Example 7 and BDR5-1 comprising H chains carrying the H429F modification (BDR5-1HF) or a 1:1 mixture of both (Isa-HF+BDR5-1HF). All mAbs were present in the cultures at 10 μg/ml and, for the mixtures, at 10 g/ml of each mAb for a total mAb concentration of 20 μg/ml. After culture for 48 h, cell viability was quantitated using a CCK8 colorimetric cell viability assay where absorbance (Abs450 nm) of the supernatant of the CCK8 treated cells is a measure of cell survival and viability. Maximum cell viability was determined by the culture of cells in the absence of antibody (no Ab) and maximum cell death by cell incubation with SDS before CCK8 treatment of the cells. Survival is represented by: [experimentally determined Abs450 nm−background Abs450 of cell culture medium], the mean Abs450 and the four replicate values are shown;

FIG. 47 provides results showing that H429F substitution confers complement-dependent lysis (CDC) of leukaemia cells resistant to lysis by the unmodified anti-CD38 mAb mezagitamab. CDC by complement-dependent lysis was determined using Ramos lymphoma cells which were opsonised especially with the mezagitamab-WT mAb or mezagitamab-H429F mutant mAb at the indicated concentrations. Background lysis was determined in the absence of mAb but in the presence of only complement (C′ only). The CDC (% killing) was determined by flow cytometry using Zombie Green;

FIG. 48 provides results showing that additional mutations of K439E or S440K in the Fc region component may suppress the enhanced CDC of H429F-modified rituximab and that the combination of rituximab-H429F/K439E with rituximab-H429F/S440K restores efficient CDC killing of Ramos cells. (A) Ramos lymphoma cells were opsonised with the individual rituximab-WT (WT) or rituximab-H429F (H429F) mAbs or with rituximab-H429F comprising the additional H chain mutation K439E (H429F/K439E) or with rituximab-H429F comprising the additional H chain mutation S440K (H429F/S440K), or with pairwise mixtures of rituximab-H429F/S440K with rituximab-H429F/K439E (H429F/K439E+H429F/S440K). Background lysis was determined in the absence of mAb but in the presence of only complement (no mAb C′ only). The CDC (% killing) of the mAbs was measured in the presence of a 1/3 dilution of human serum by flow cytometry using Zombie green. The enhanced CDC mediated by H429F modification of rituximab was suppressed by either Fc:Fc interactions inhibiting the K439E or S440K mutations, but was found to be fully recovered by the mixture of both IgG mutants. (B) For the binding analysis, the proteins were serially two-fold titrated and binding activity (on Ramos cells) quantified by flow cytometry using a FITC-conjugated anti-hIgG-Fc secondary reagent. The level of non-specific background binding of fluorescent conjugate to cells is shown (conj only); MFI=Median Fluorescence Intensity. All mAbs gave near identical binding to CD20;

FIG. 49 provides the results of a flow cytometric analysis of binding (A, C) and CDC (B, D) of purified flACE2-Fc fusion protein (A, B) and SARS-CoV spike-specific mAbs (C, D) using Ramos-S cells. For the binding analysis, the proteins were serially two-fold titrated and binding activity (on Ramos-S cells) quantified by flow cytometry using a FITC-conjugated anti-hIgG-Fc secondary reagent. The level of non-specific background binding of fluorescent conjugate to cells is shown (conj); MFI=Median Fluorescence Intensity. For CDC analysis, the proteins were serially two-fold titrated and human serum used as a source of complement. The CDC lysis was quantitated using Zombie Green. Background lysis by complement in the absence antibodies was determined and is shown (no mAb C′ only);

FIG. 50 provides graphical results from flow cytometric analysis showing that cooperative and functional synergy of the H429F-modified flACE2-Fc fusion protein and H429F-modified anti-SARS-CoV-2 mAbs further enhances complement dependent killing (CDC). (A) The mAbs S2P6-H429F (S2P6-HF) or S2P6-WT were titrated alone or titrated in the presence of a fixed concentration of the flACE2-Fc-H429 fusion protein (1 μg/ml final concentration) (S2P6-HF+flACE2-Fc-HF) that, when used alone, mediated CDC killing of 23.4% (indicated by open diamond symbol), or were titrated in the presence of fixed concentration of flACE2-Fc-WT (1 μg/ml final) (S2P6-WT+flACE2-Fc-WT) which, when used alone, mediated a % kill of 5.0% (filled diamond). The background lysis by complement in the absence of mAb or Fc fusion protein is shown (C′ only); Arrow shows example of greatest synergy; (B) CDC killing potency of H429F-modified flACE2-Fc was evaluated on Ramos-S cells. The mAbs CC40.8-H429F (CC40.8-HF) and CV3-25-H429F (CV3-25-HF) were used alone (2.5 μg/ml final concentration) or mixed with flACE2-Fc-H429F (CC40.8-HF+flACE2-Fc-HF; CV3-25-HF+flACE2-Fc-HF; note that the final concentrations of the mAbs was 2.5 μg/ml and of the flACE2-Fc-H429F was 1 μg/ml). Killing potency was evaluated in the flow cytometric assay using Zombie Green. The % CDC killing mediated by flACE-2-Fc-WT and background lysis by complement in the absence of mAb or Fc fusion protein (C′ only) is also shown. Four replicate values and SEM are shown. Mean % kill value is shown above each column;

FIG. 51 provides results showing that ACE2-Fc fusion proteins in three formats, namely trACE2-Fc, flACE2Fc and EflACE2-Fc, comprising Fc region components with the wild type sequence or the H429F mutation bind equivalently to Ramos-S target cells; as determined using flow cytometric analysis using an anti-IgG secondary reagent labelled with goat anti-hIgG Fc FITC: (A) trACE2-Fc (mean±SEM, n=3); (B) flACE2-Fc (mean±SEM, n=3); and (C) EflACE2-Fc (WT n=3, H429F n=1). In all panels, the level of non-specific binding of fluorescent conjugate to cells is shown (conj only); MFI=Median Fluorescence Intensity. The EC50 (nM) values from the curve fits are shown;

FIG. 52 provides results showing that the ACE2-Fc fusion proteins in three formats (trACE2-Fc, flACE2-Fc and EflACE2-Fc) comprising Fc region components with the wild type sequence or the H429F mutation, strongly fix complement and direct complement dependent cytotoxicity (CDC) of Ramos-S target cells; as determined using flow cytometric analysis of CDC of opsonised Ramos-S cells using diluted normal human serum as a source of complement: (A) trACE2-Fc (mean±SEM, n=3); (B) flACE2-Fc (mean±SEM, n=3); (C) EflACE2-Fc (WT n=3, H429F n=1). In all panels, the level of non-specific complement killing in the absence of ACE2-Fc fusion proteins is shown complement only (C′ only). The EC50 (nM) values from the curve fits are shown for the H429F proteins. Fits for the WT proteins could not be determined (nd);

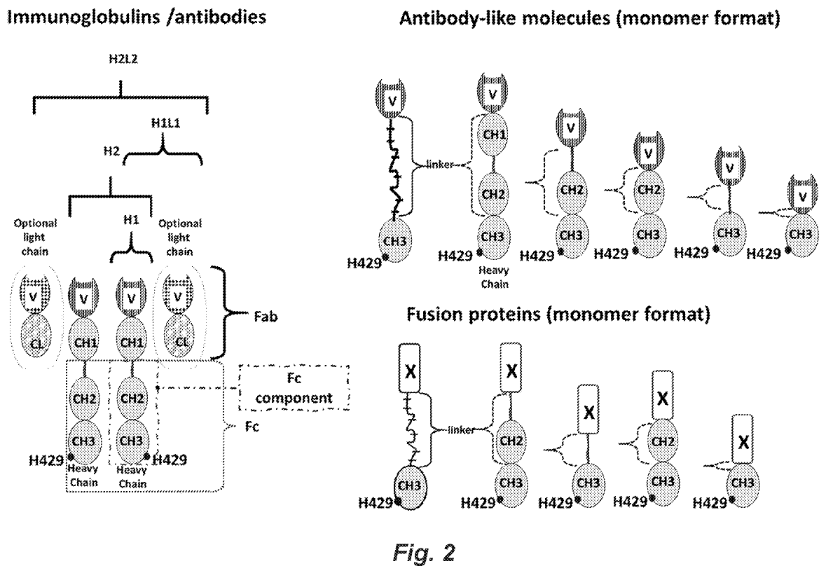

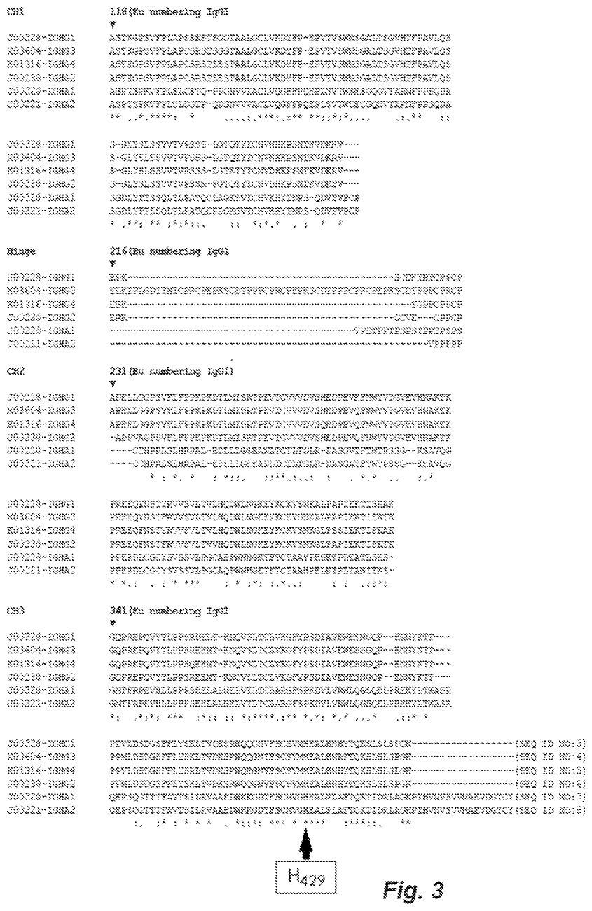

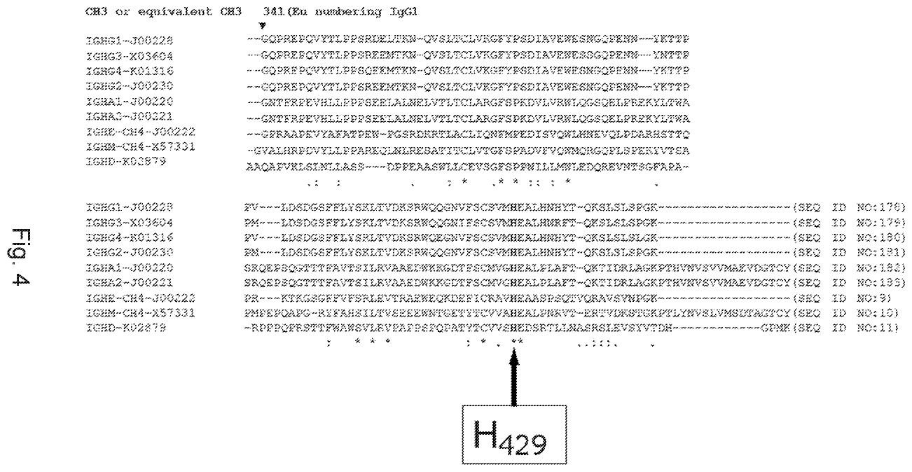

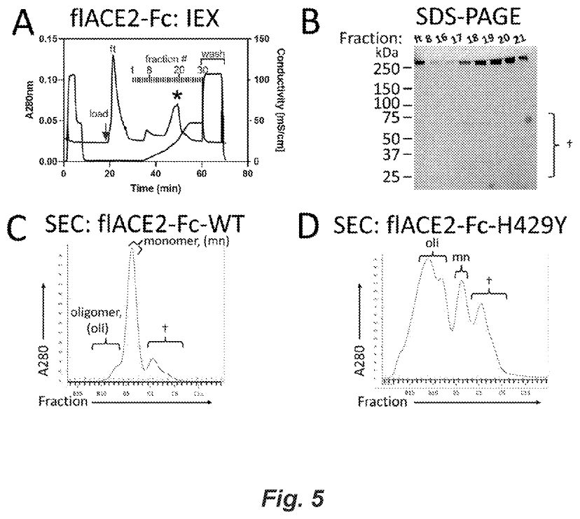

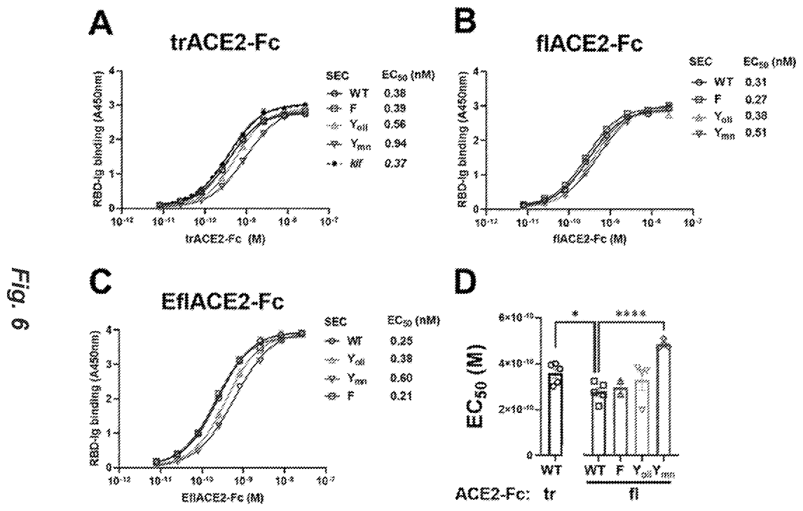

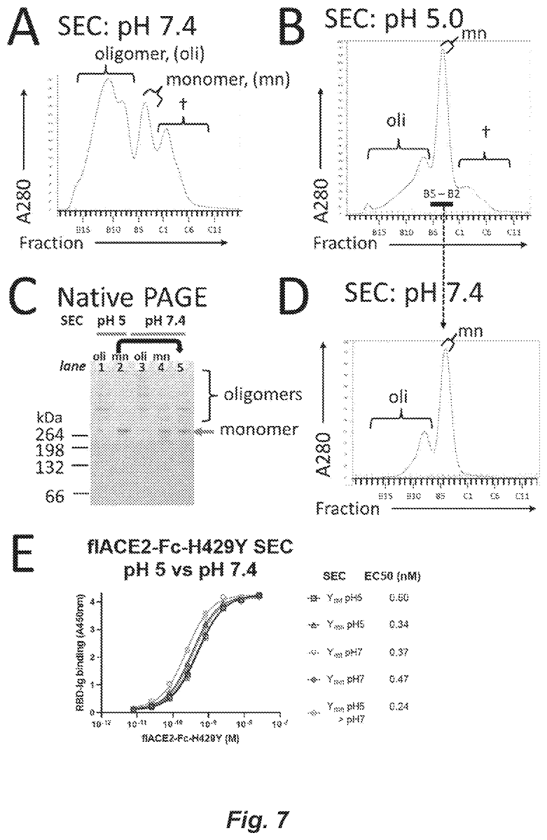

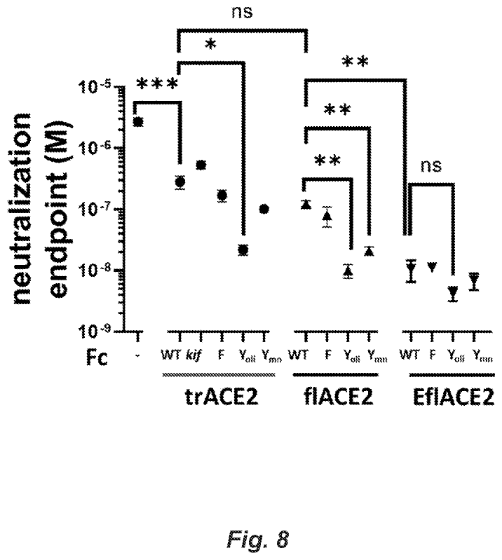

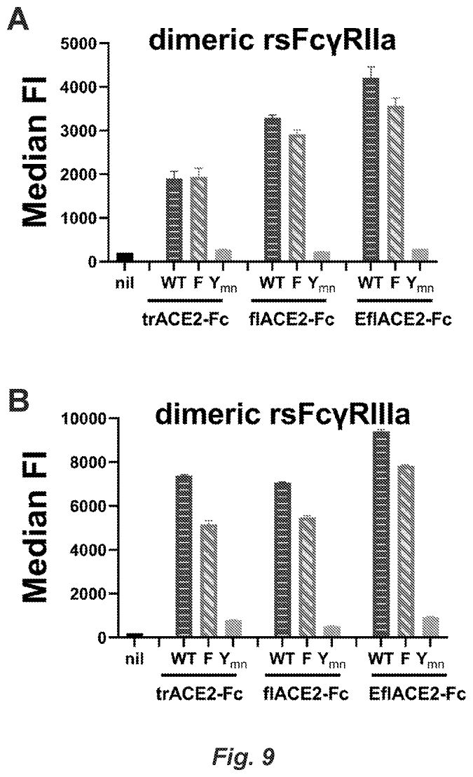

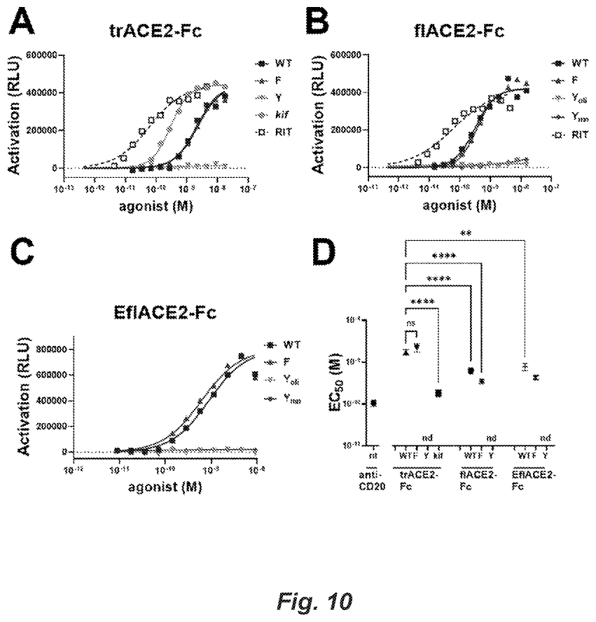

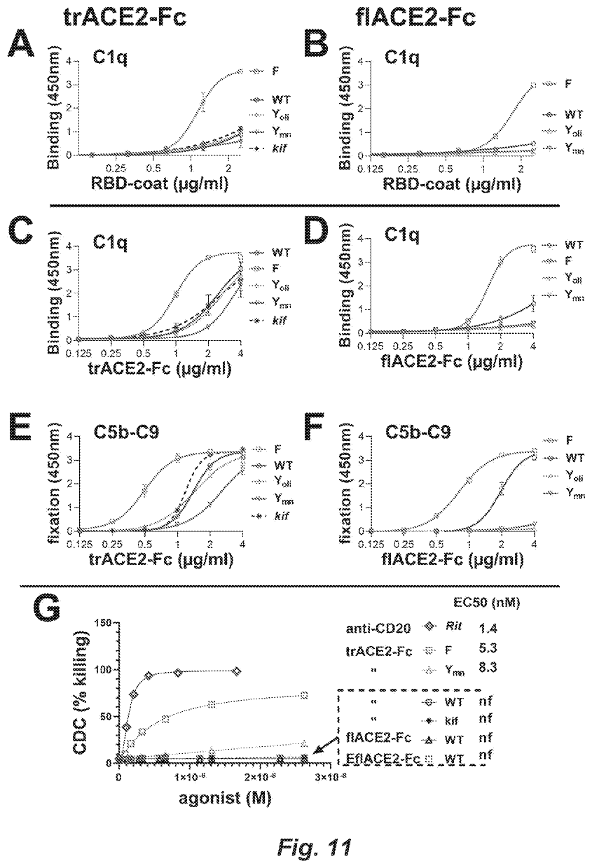

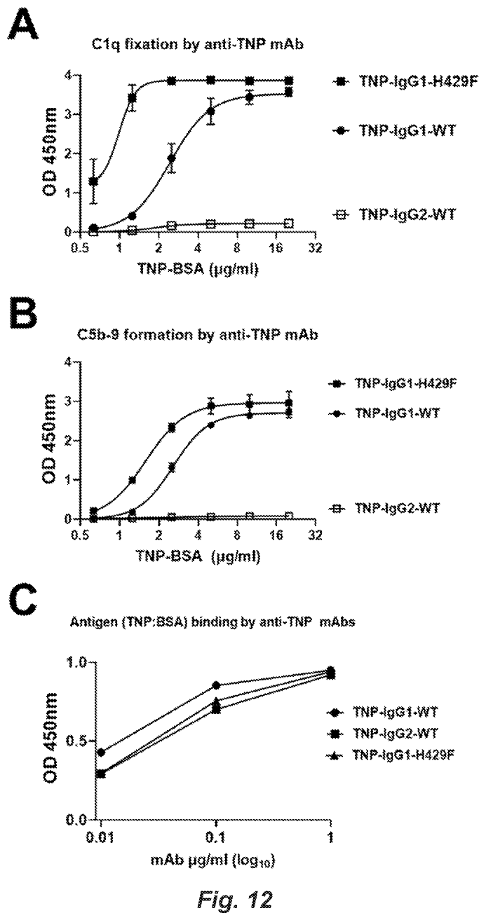

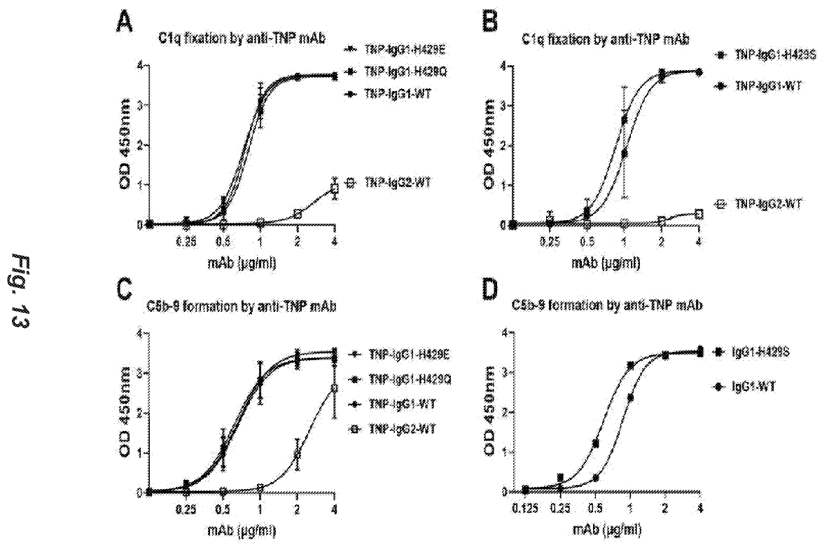

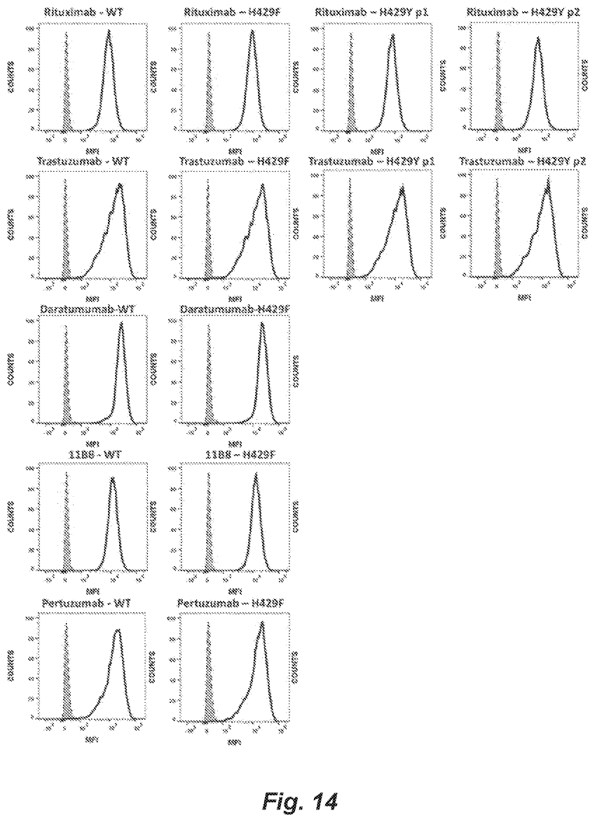

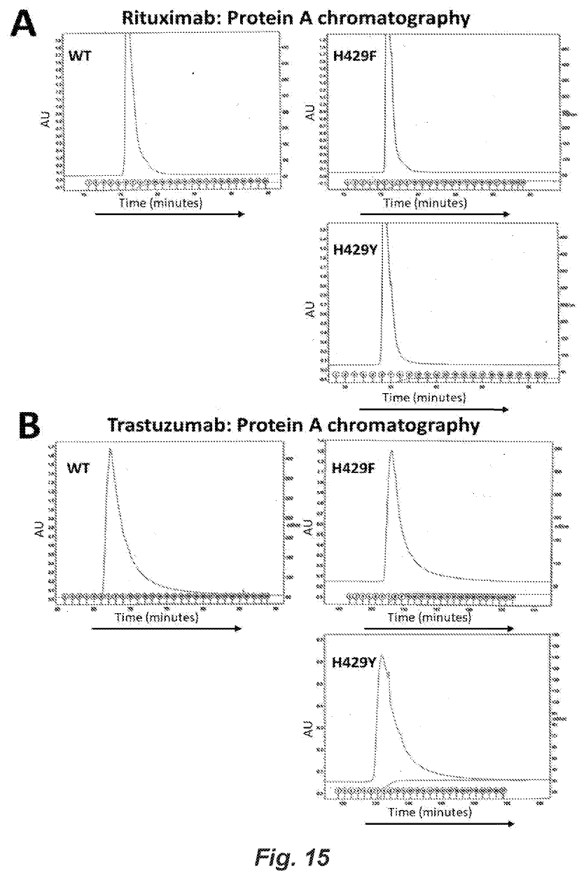

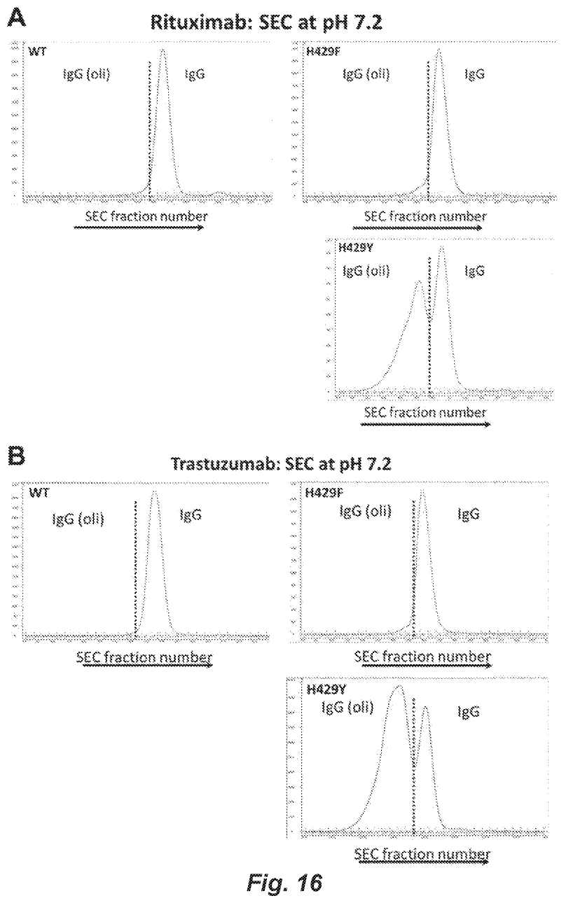

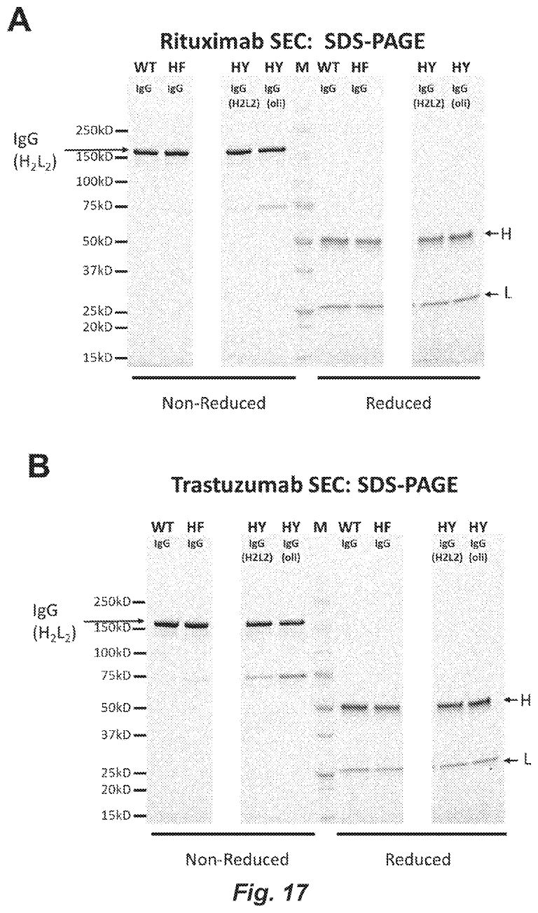

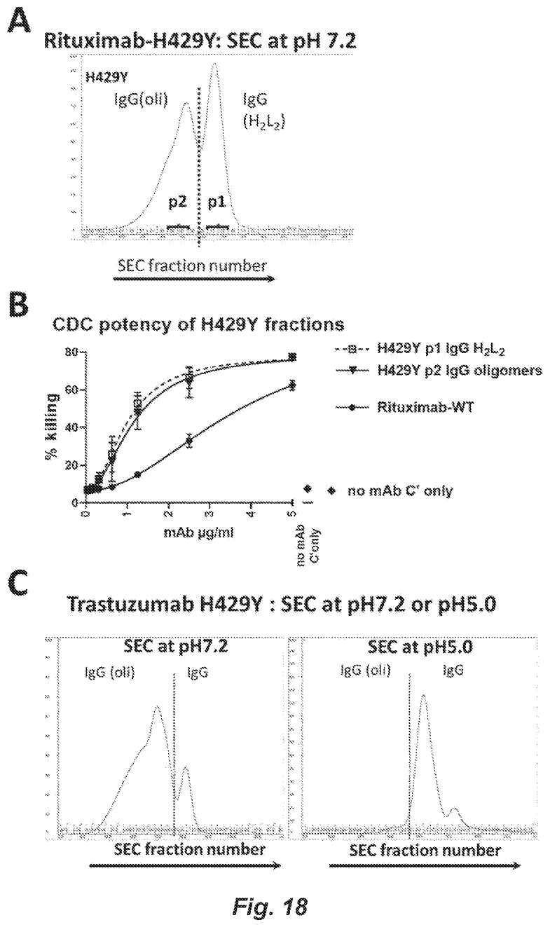

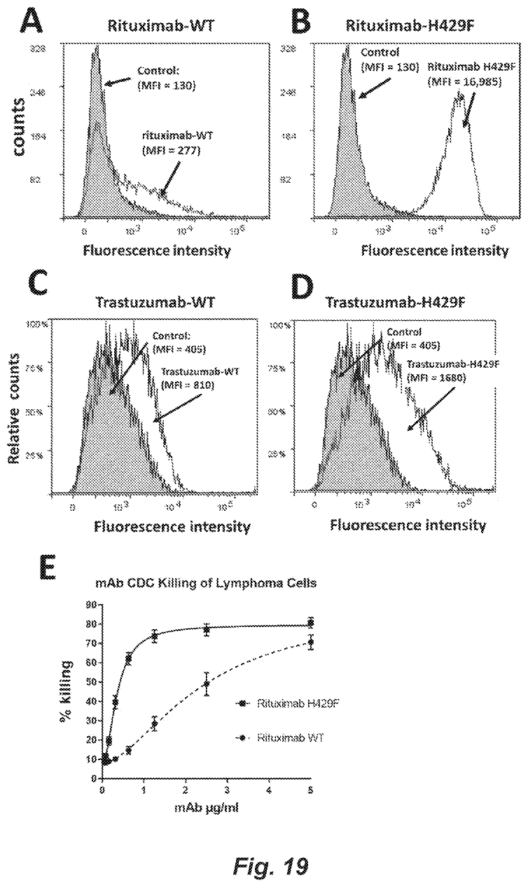

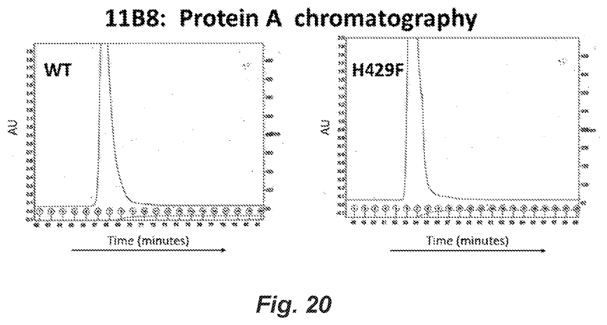

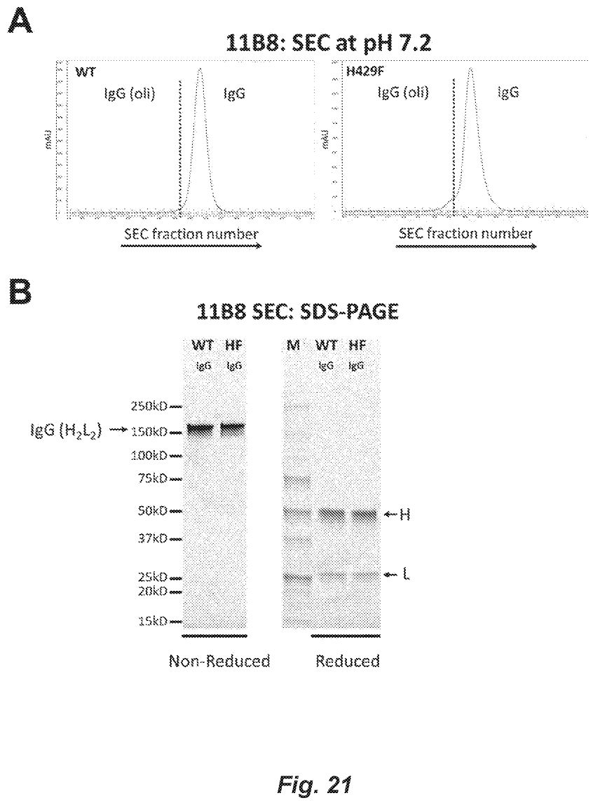

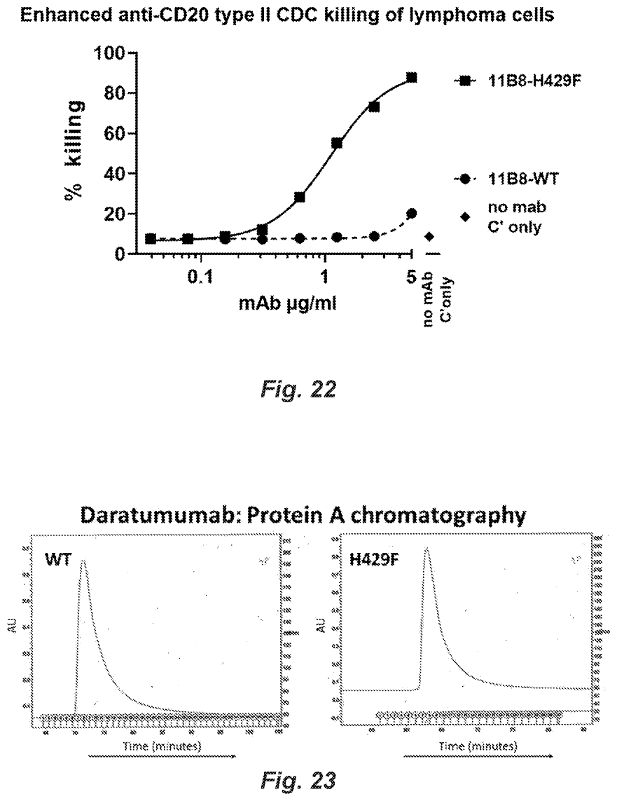

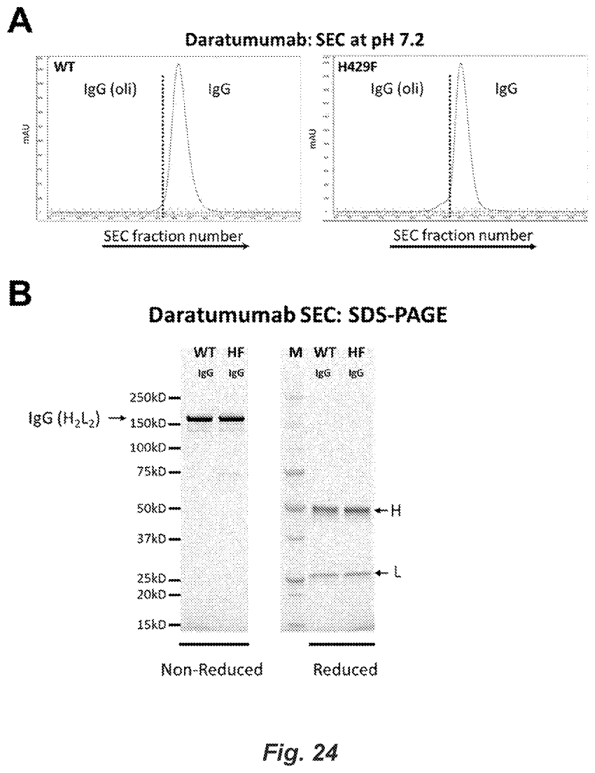

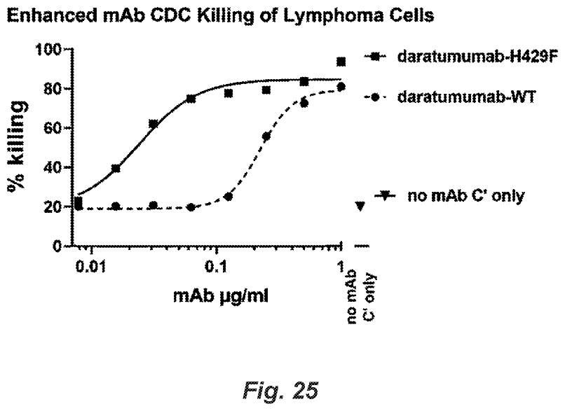

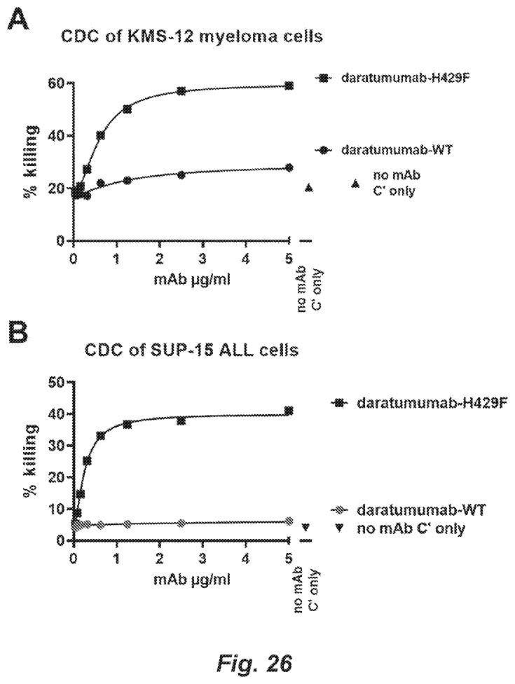

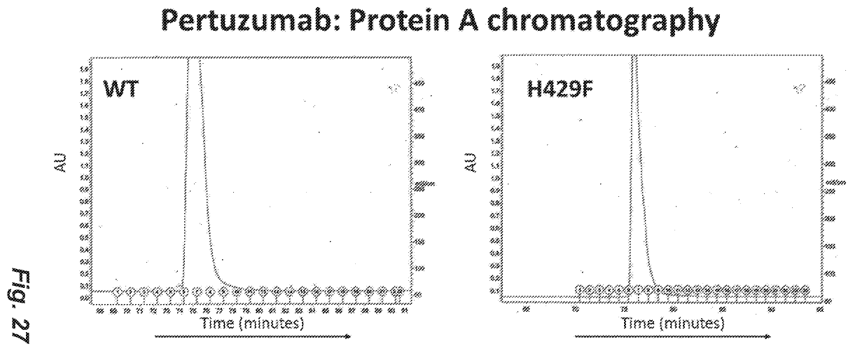

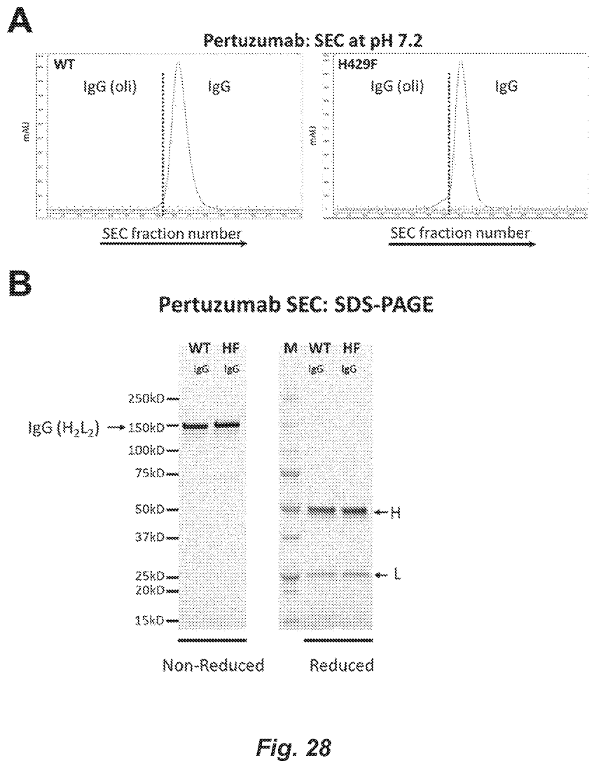

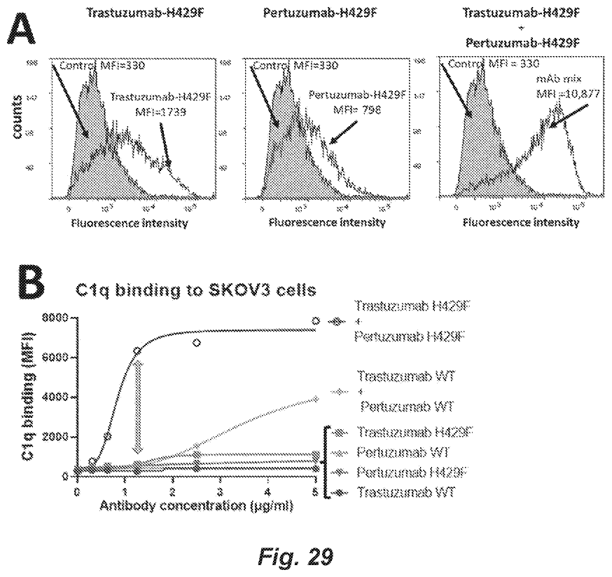

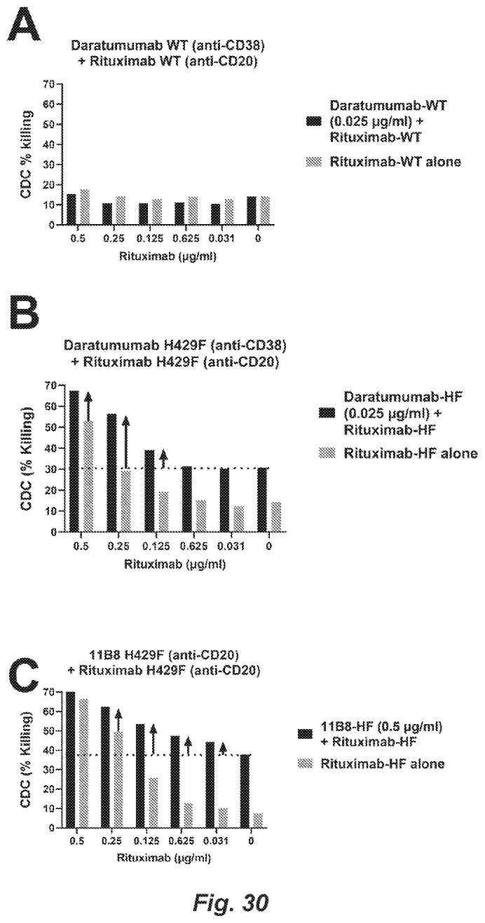

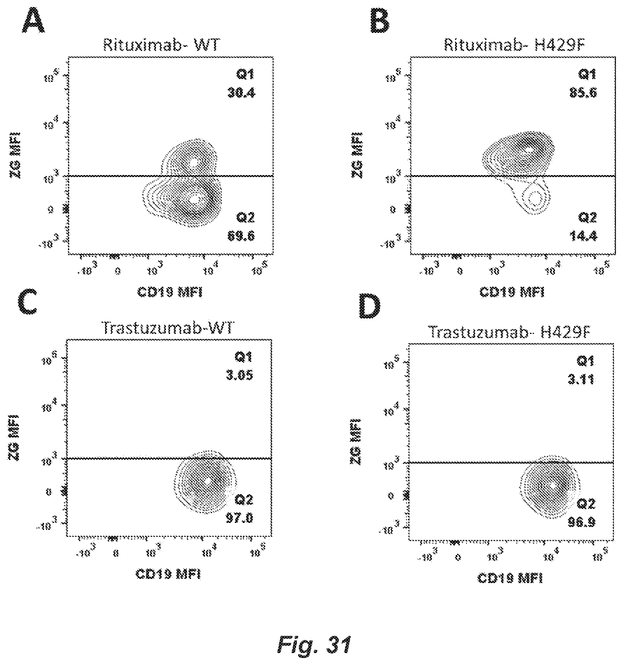

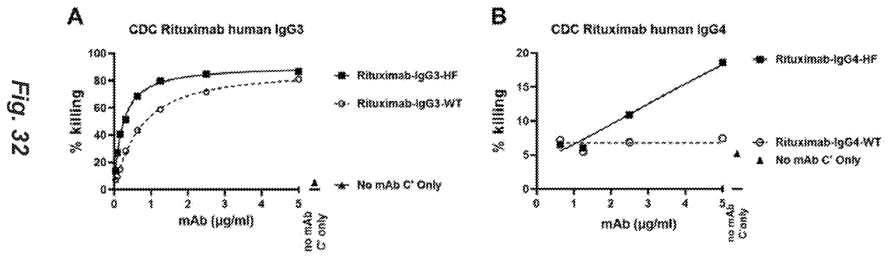

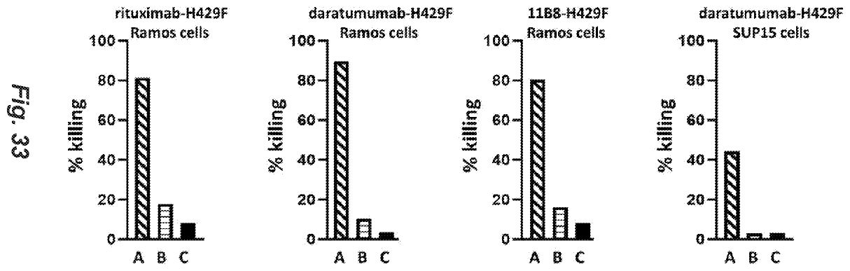

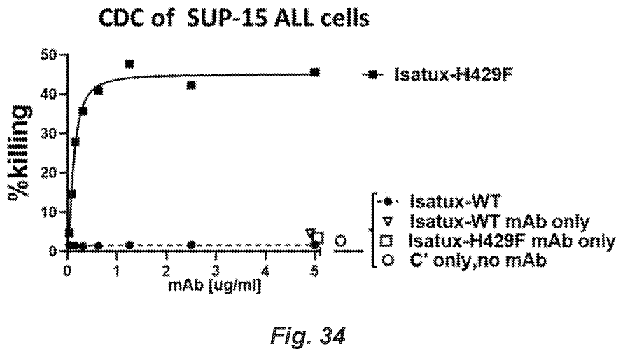

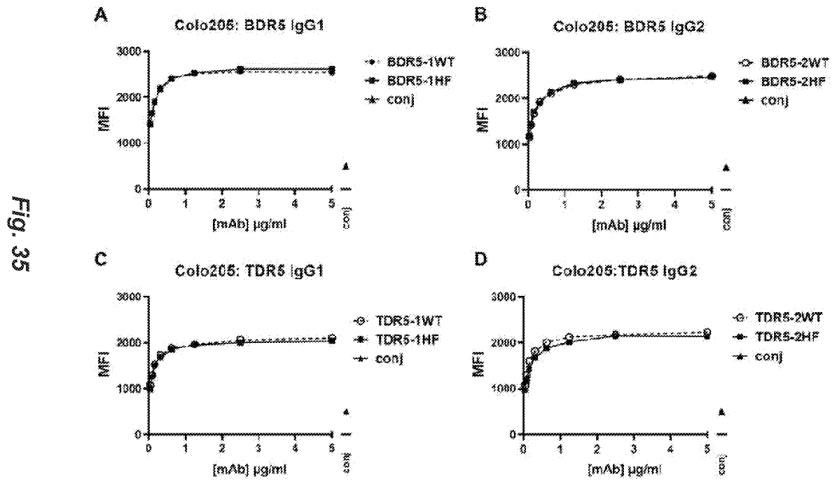

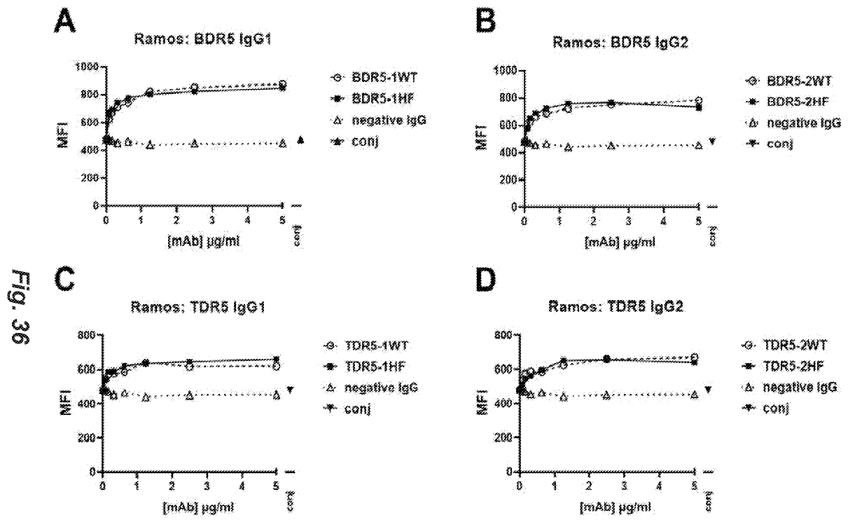

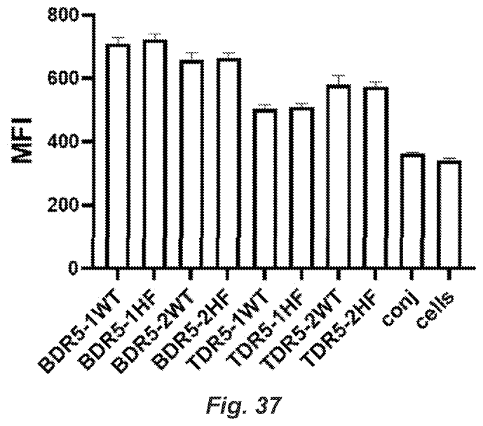

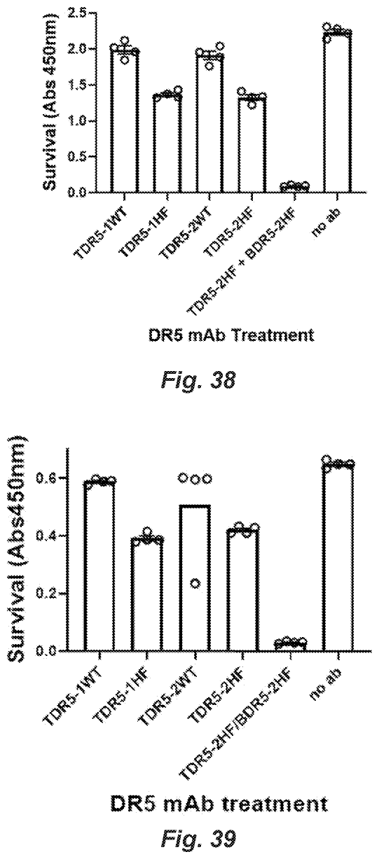

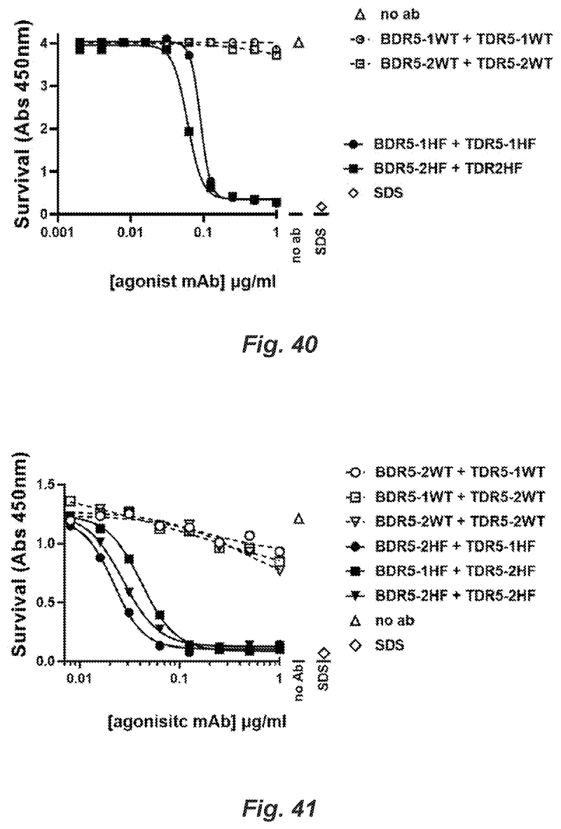

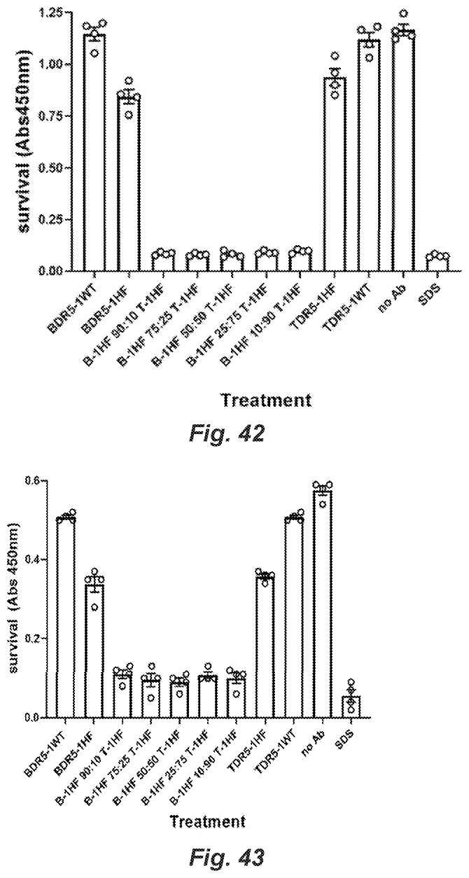

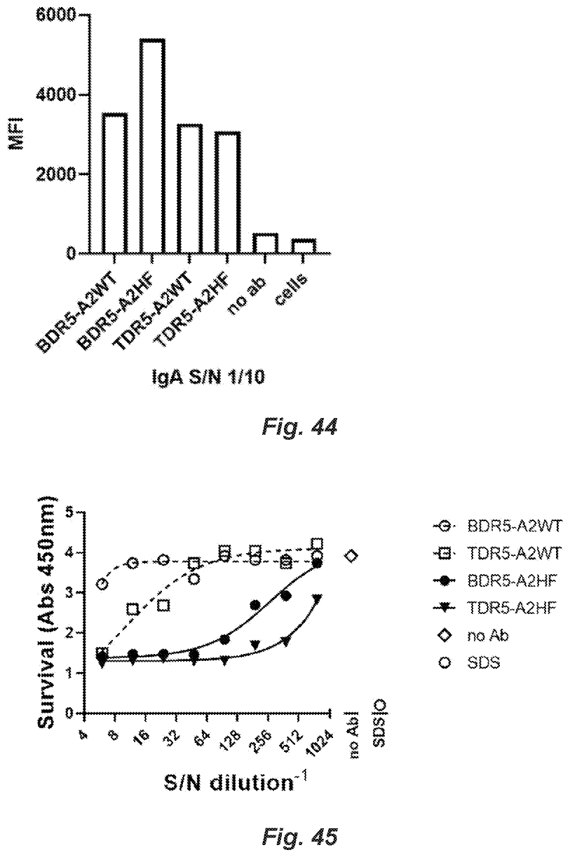

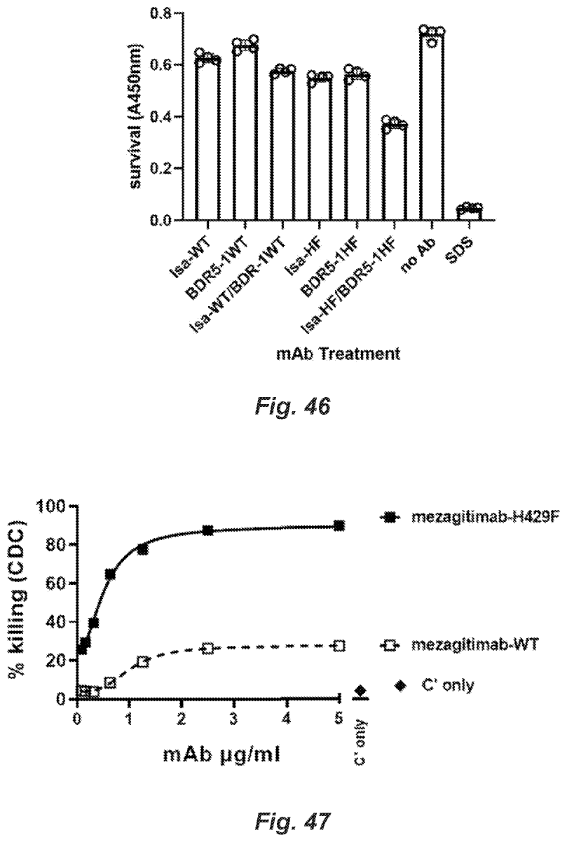

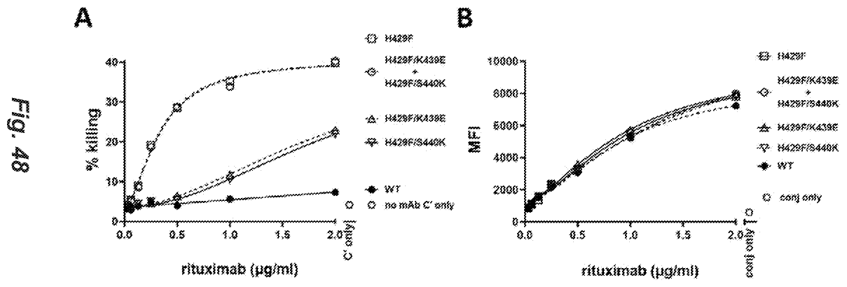

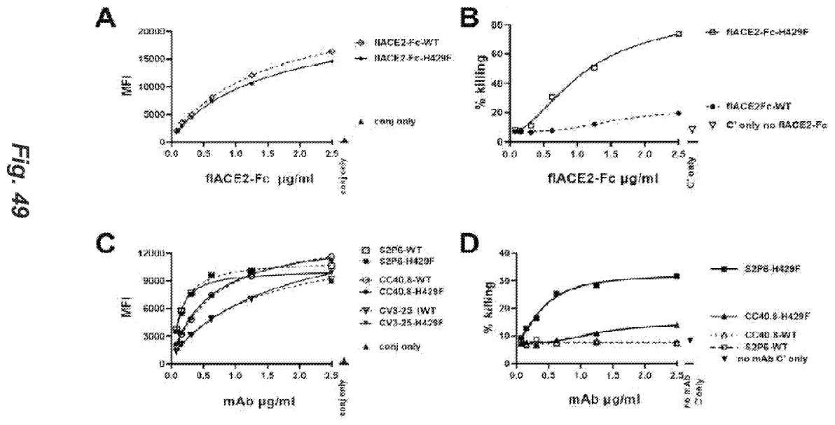

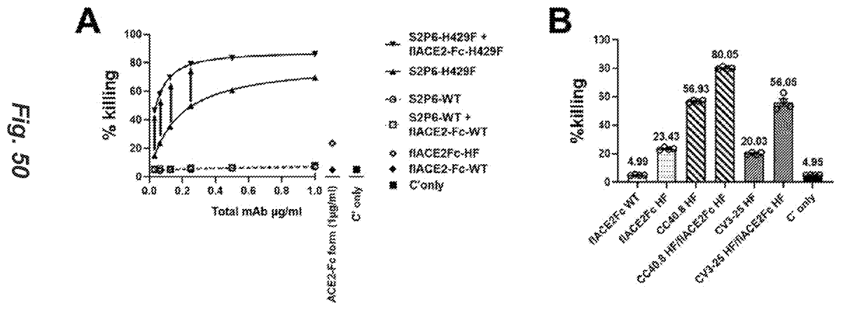

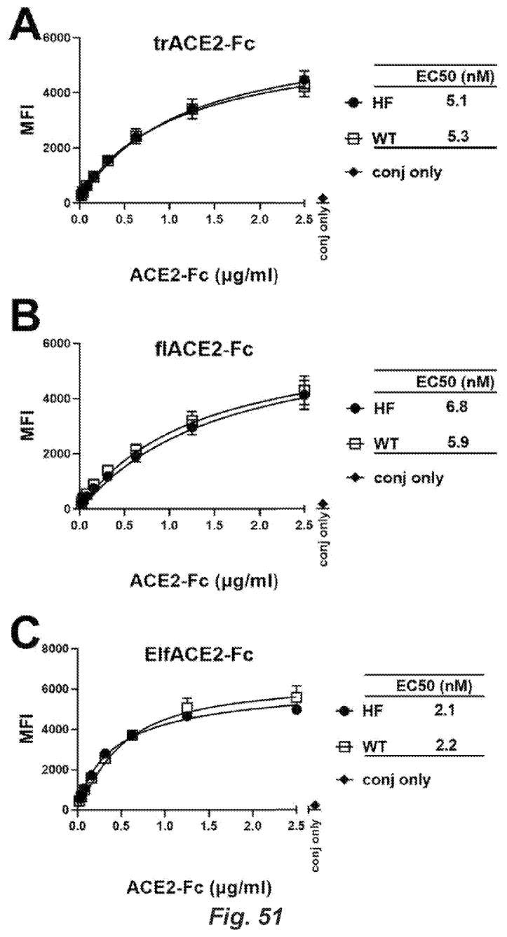

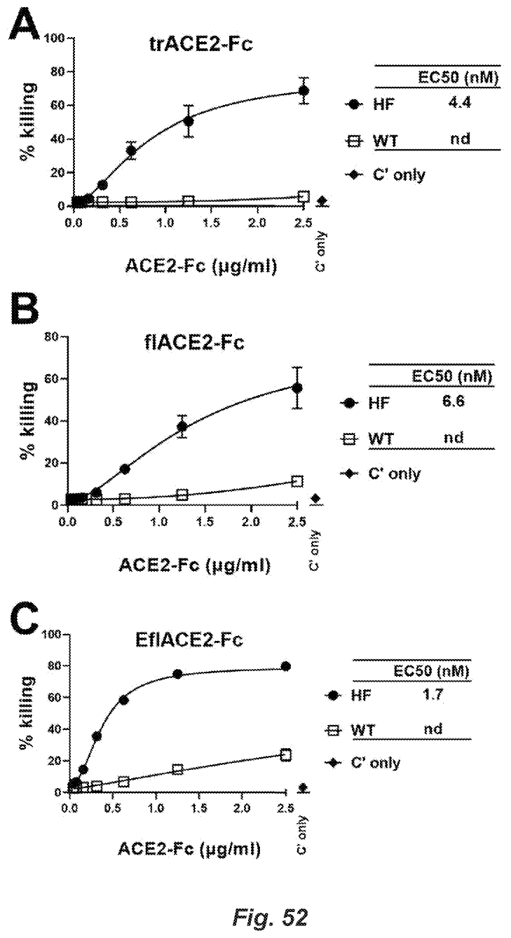

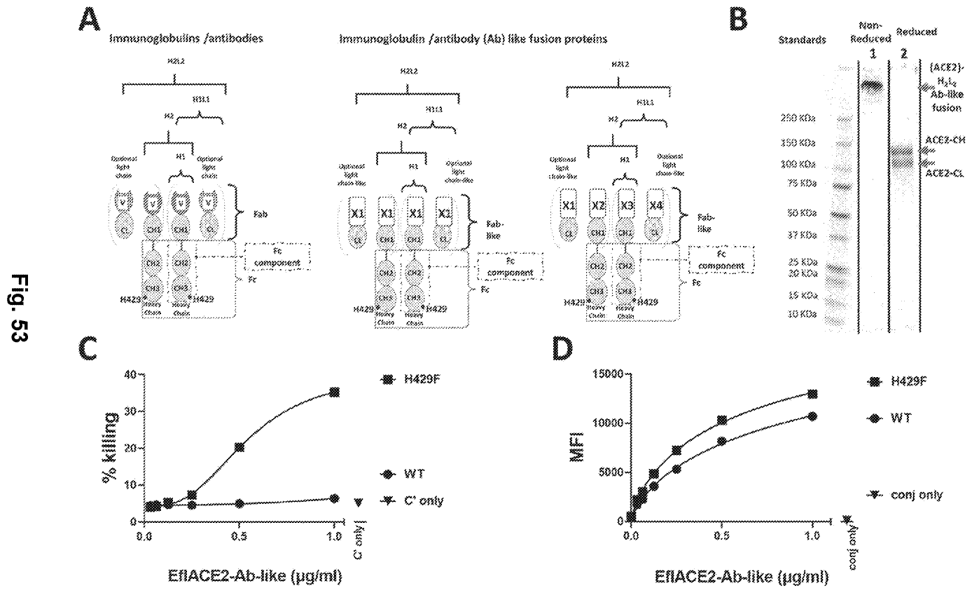

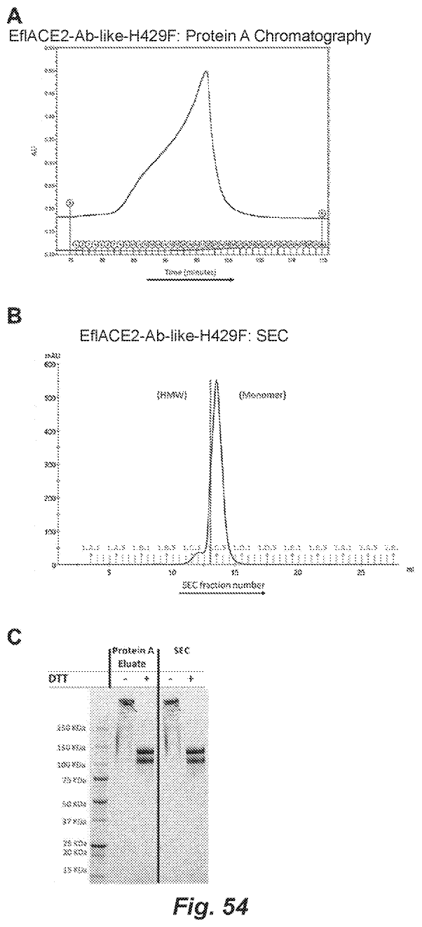

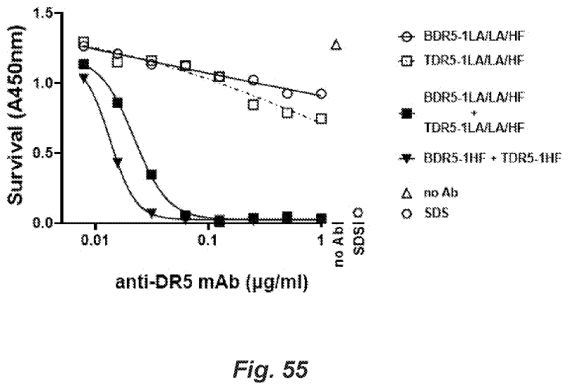

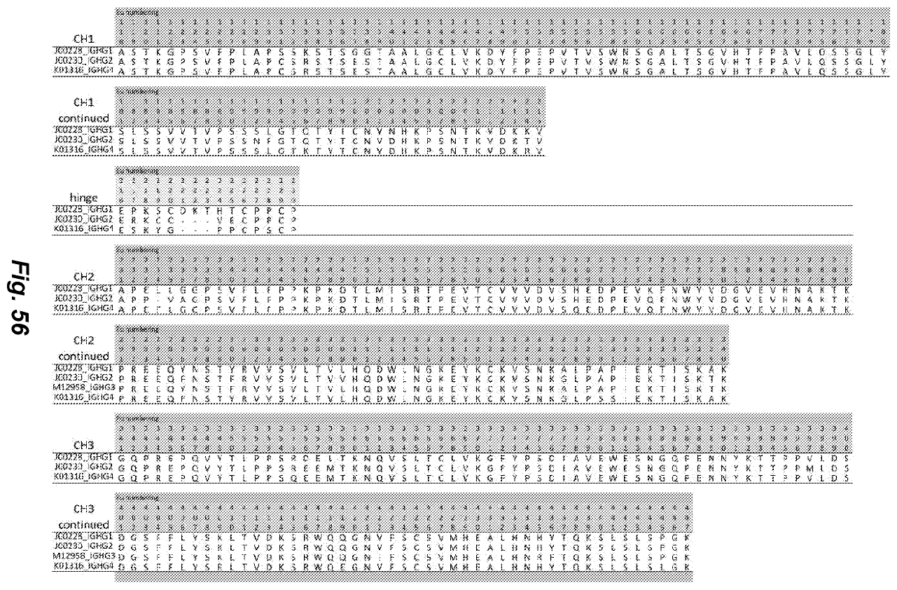

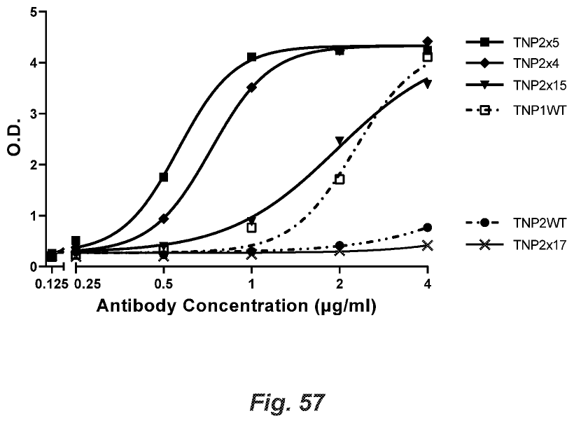

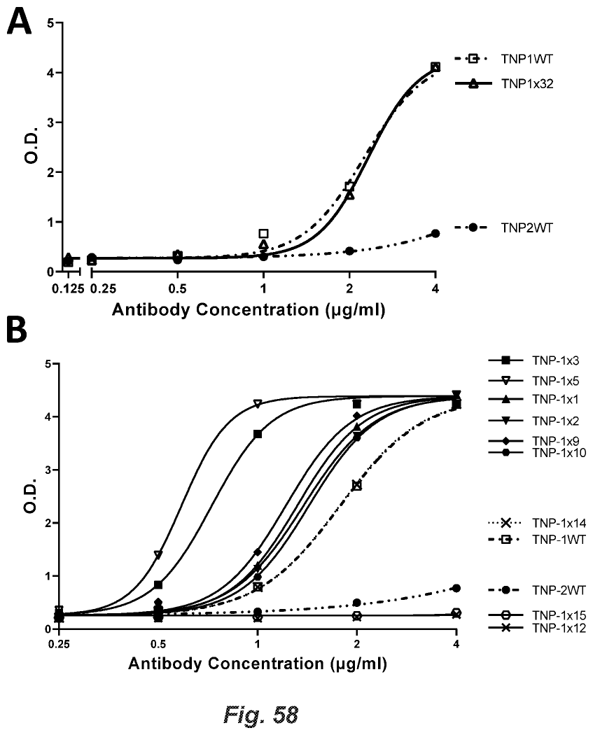

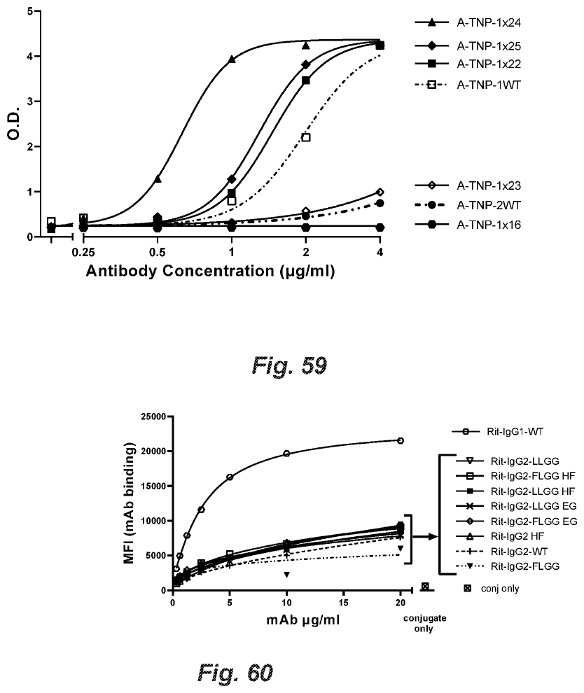

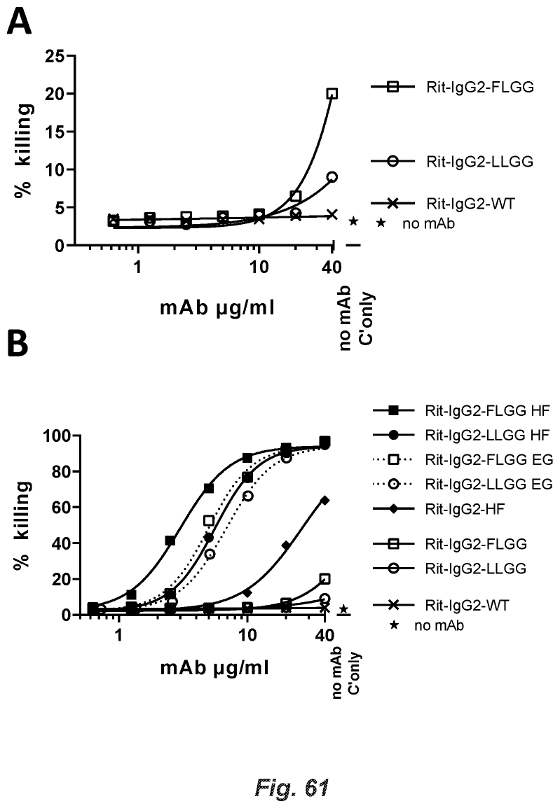

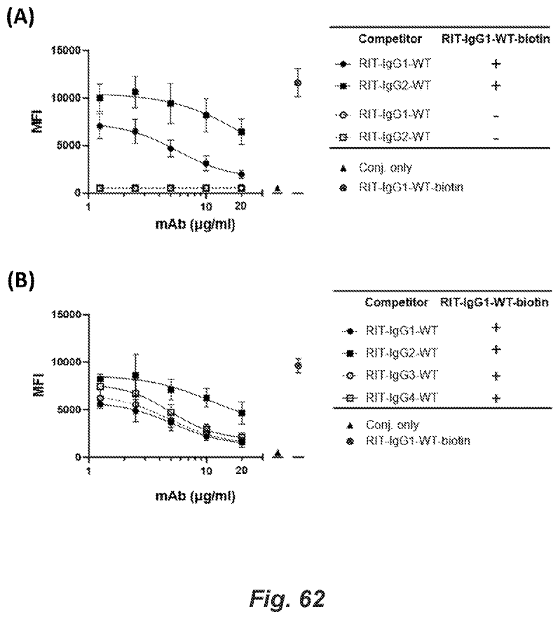

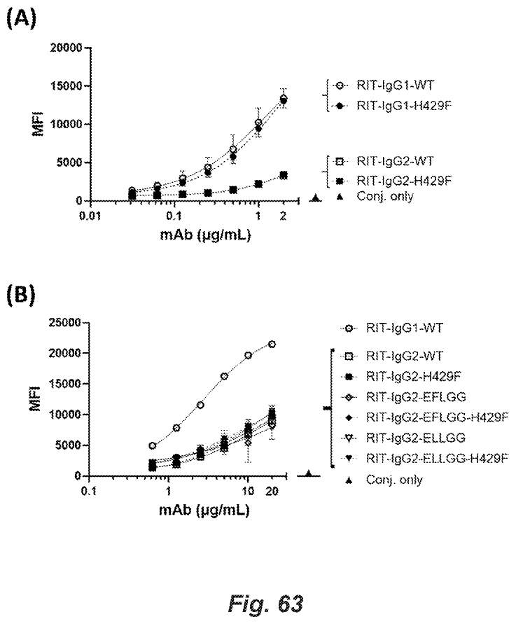

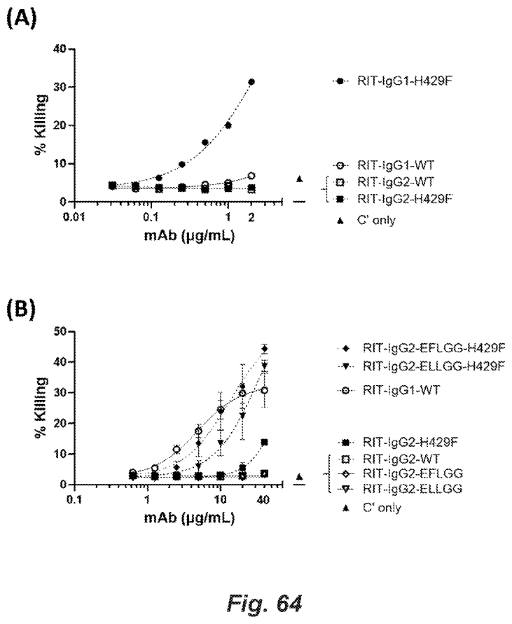

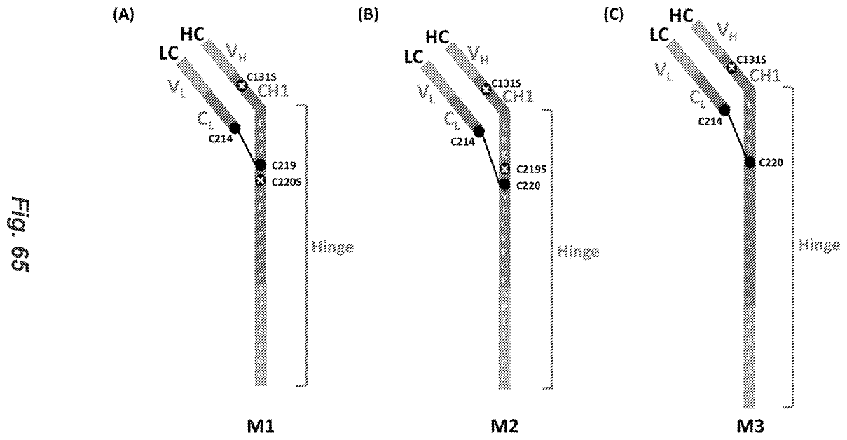

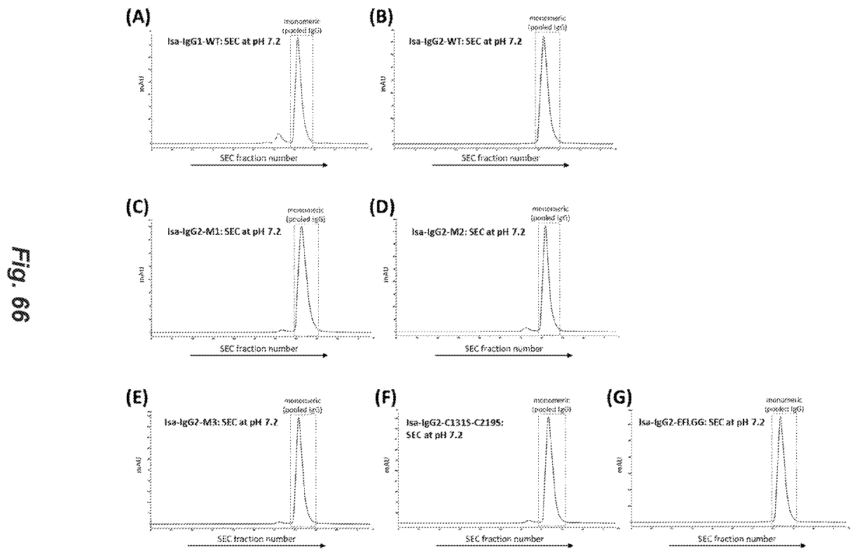

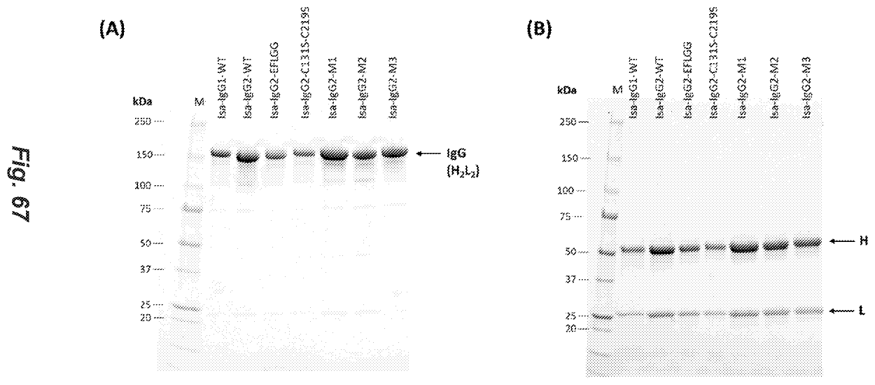

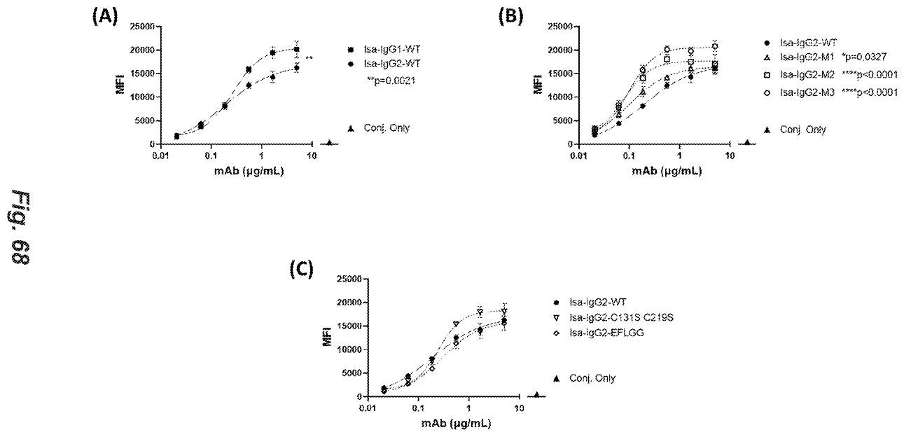

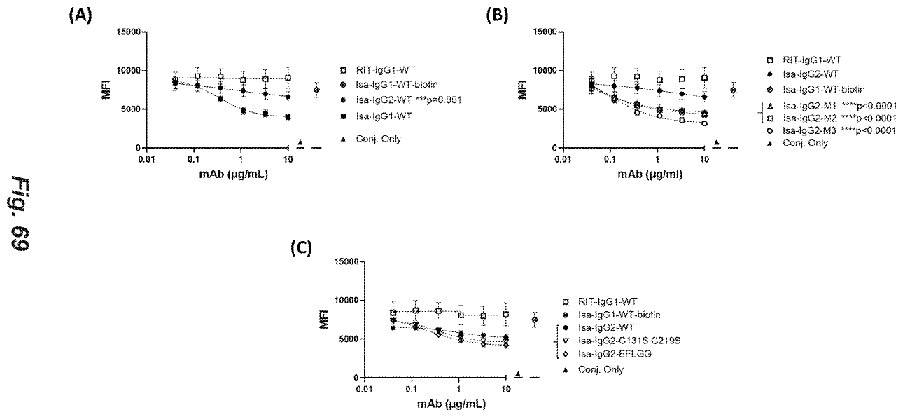

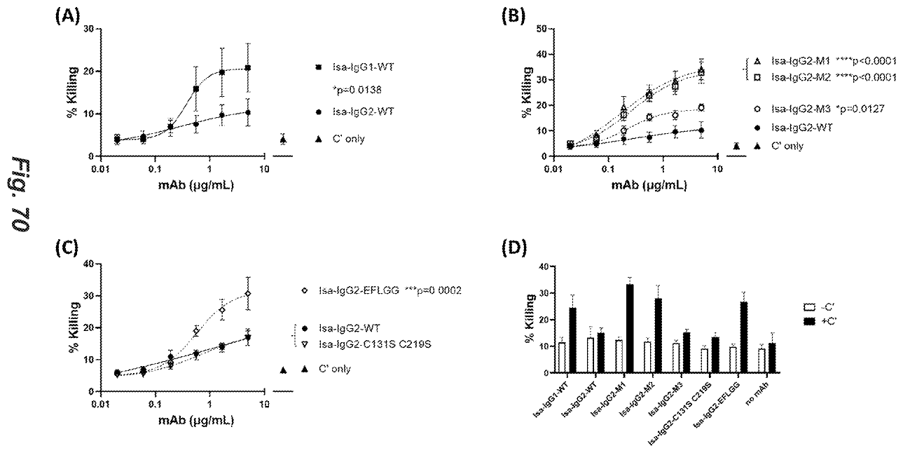

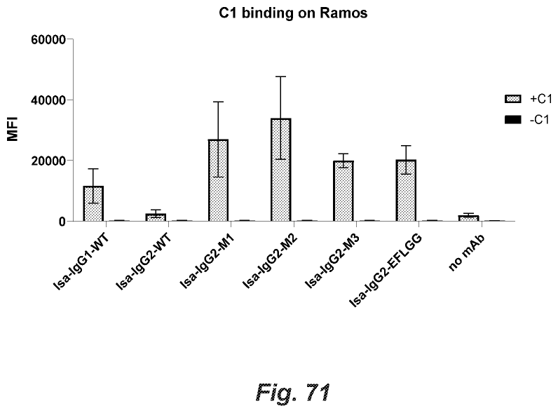

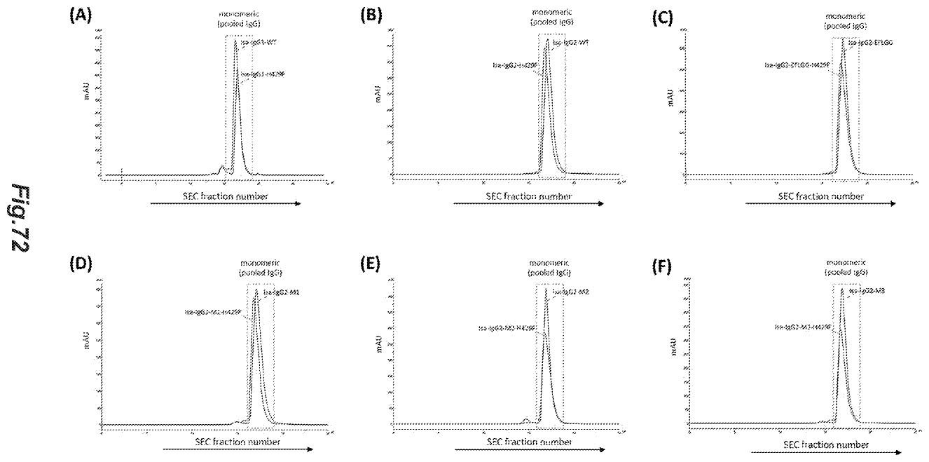

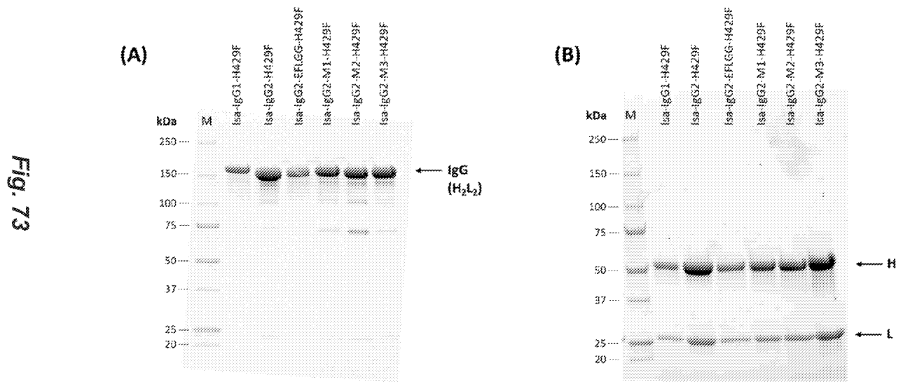

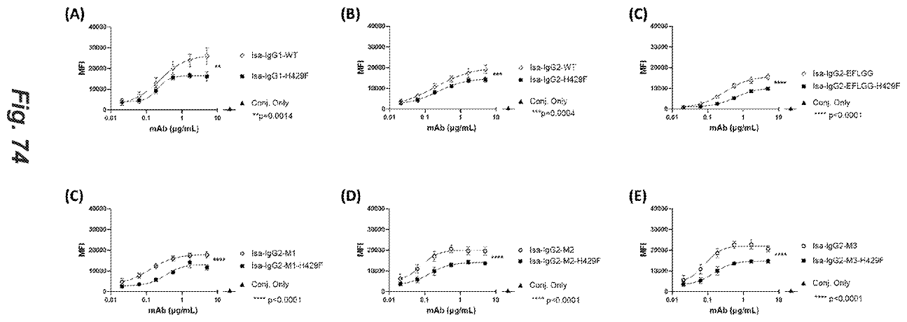

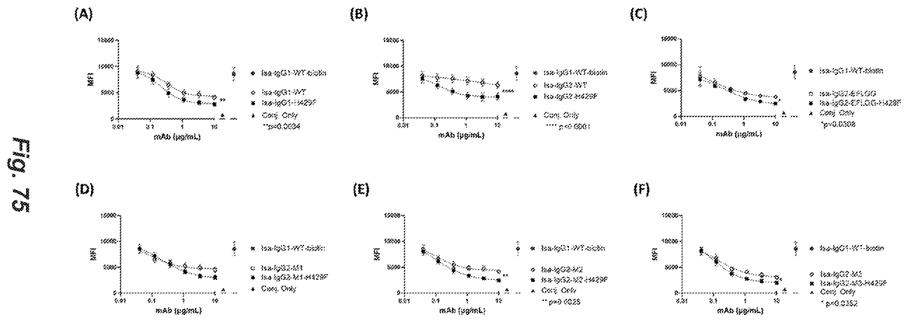

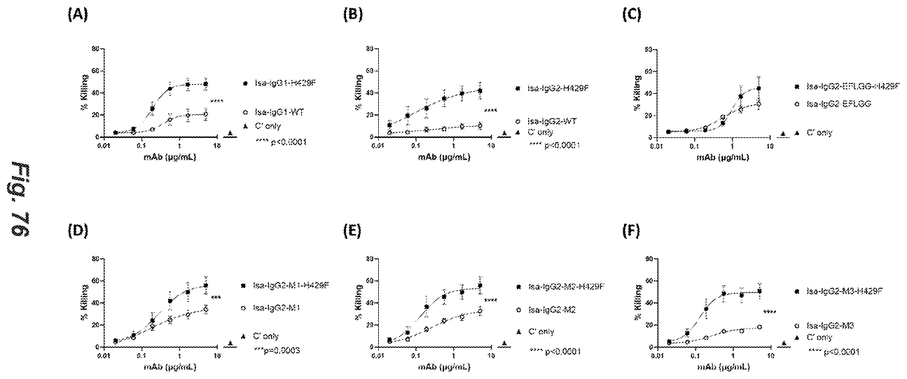

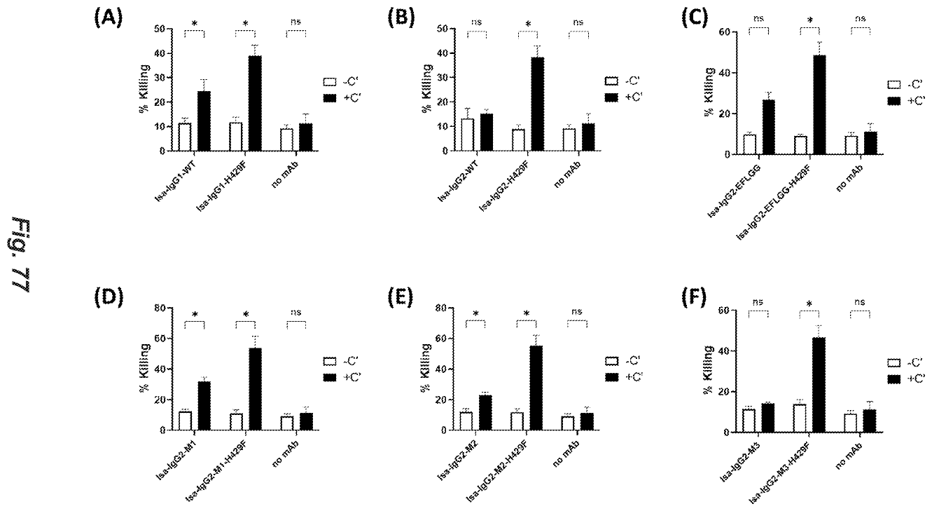

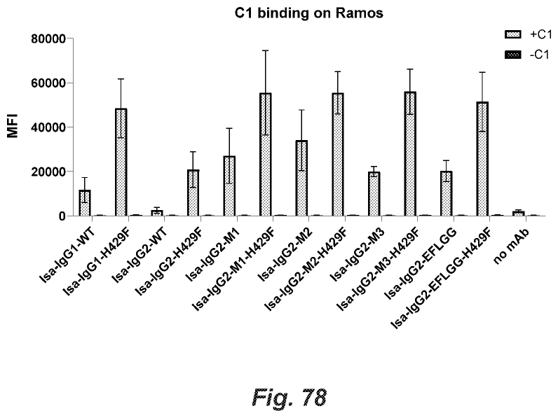

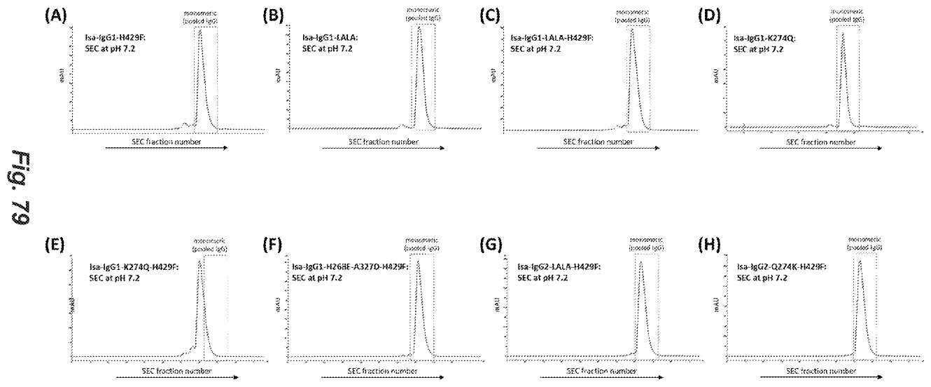

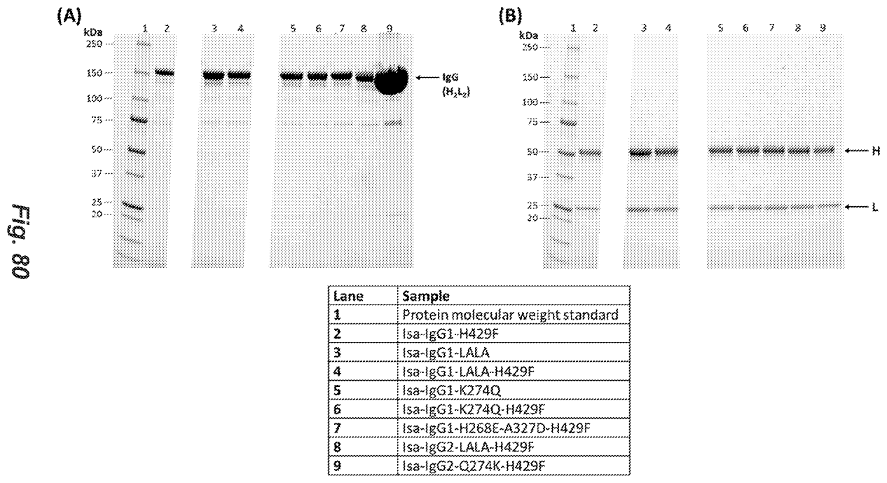

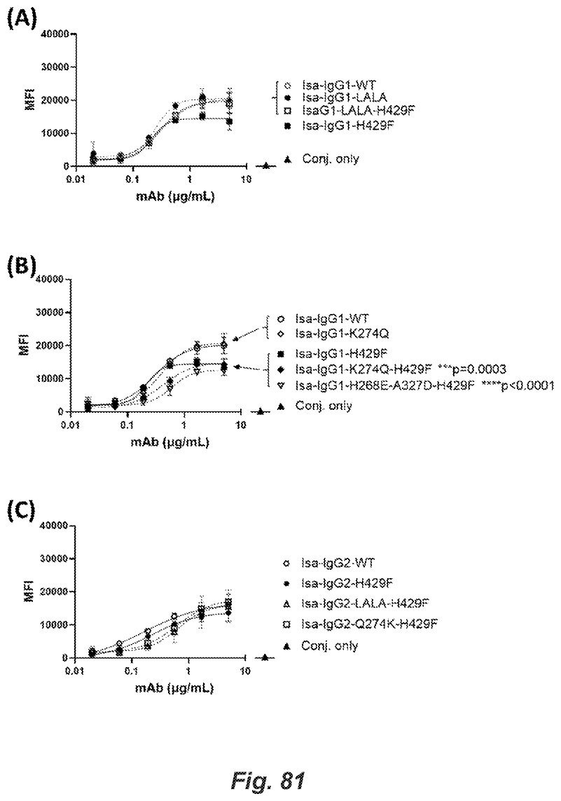

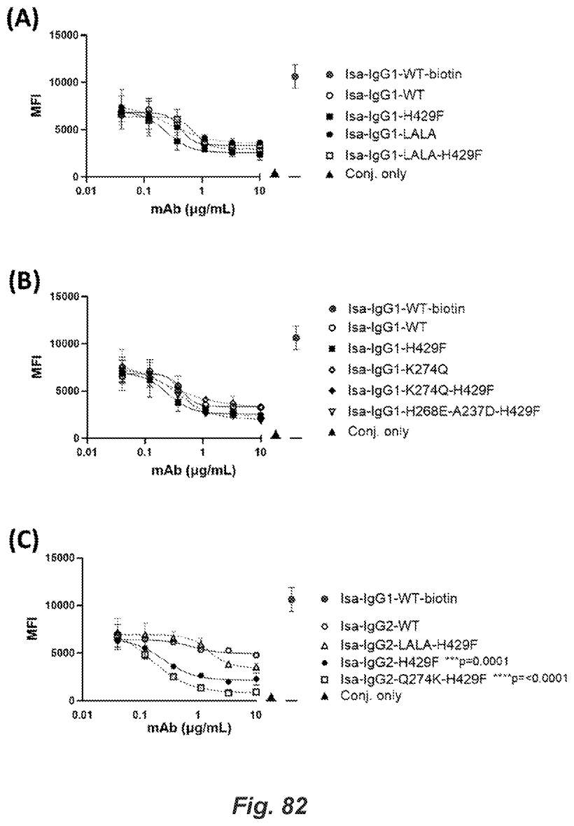

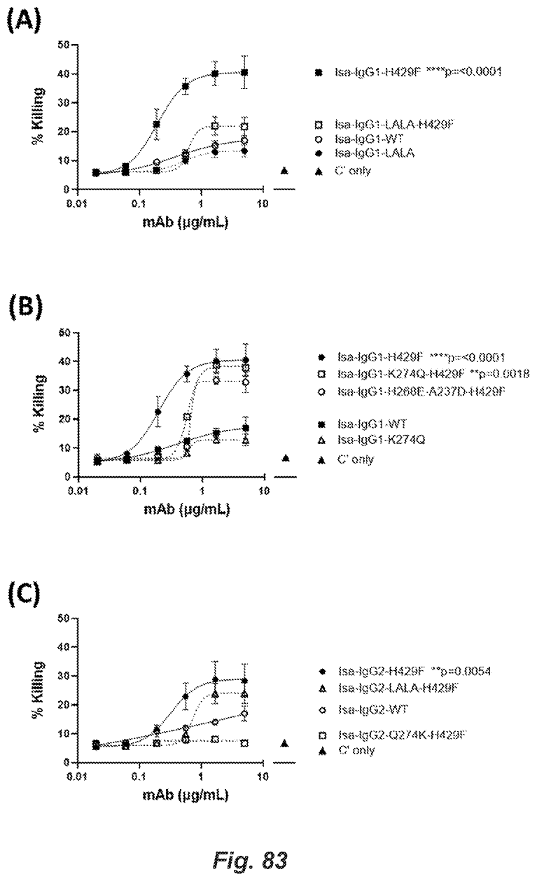

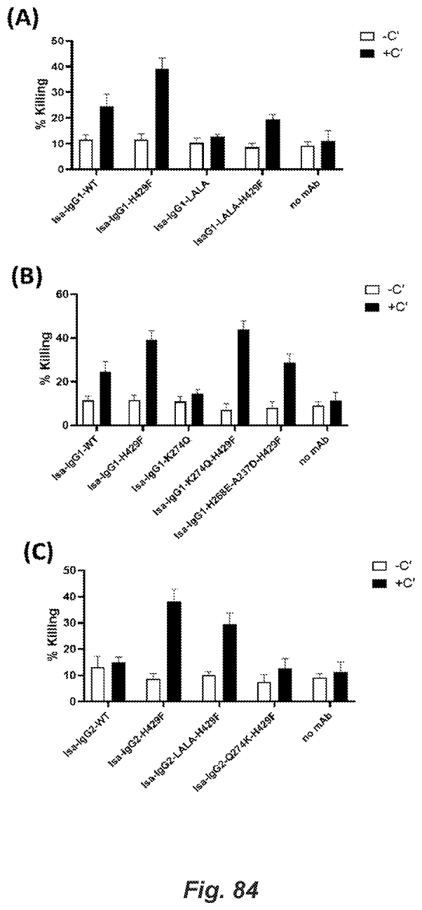

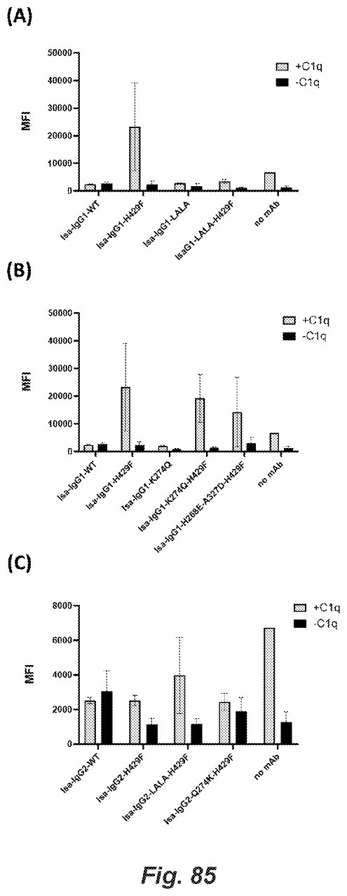

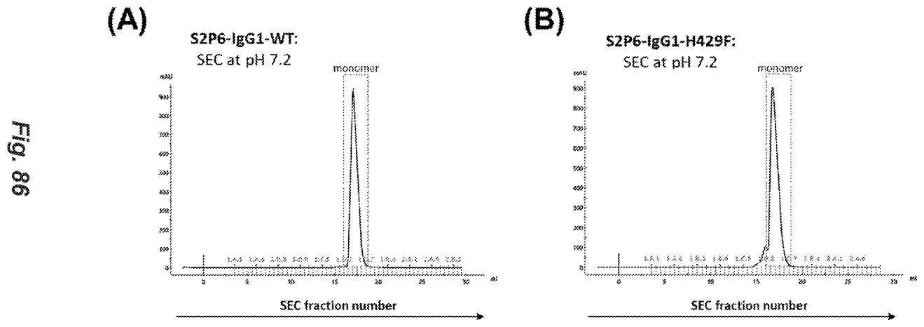

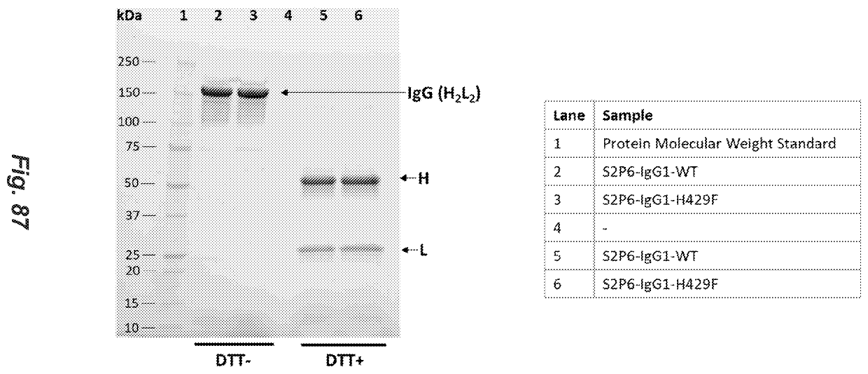

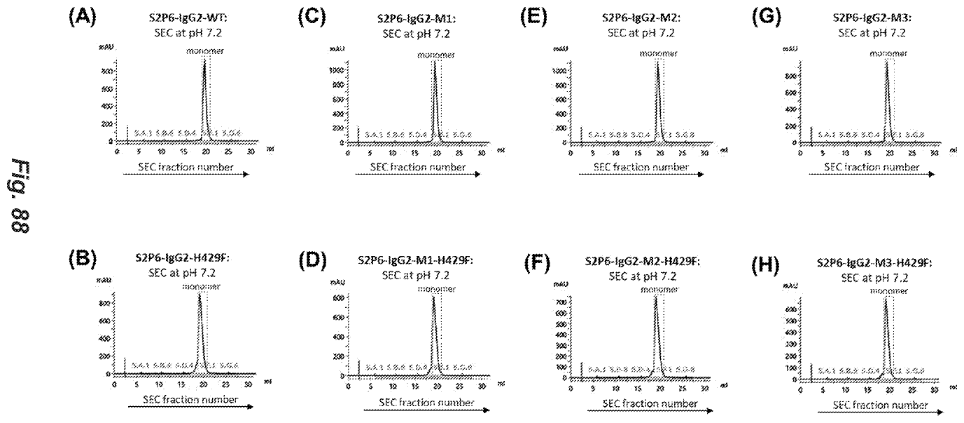

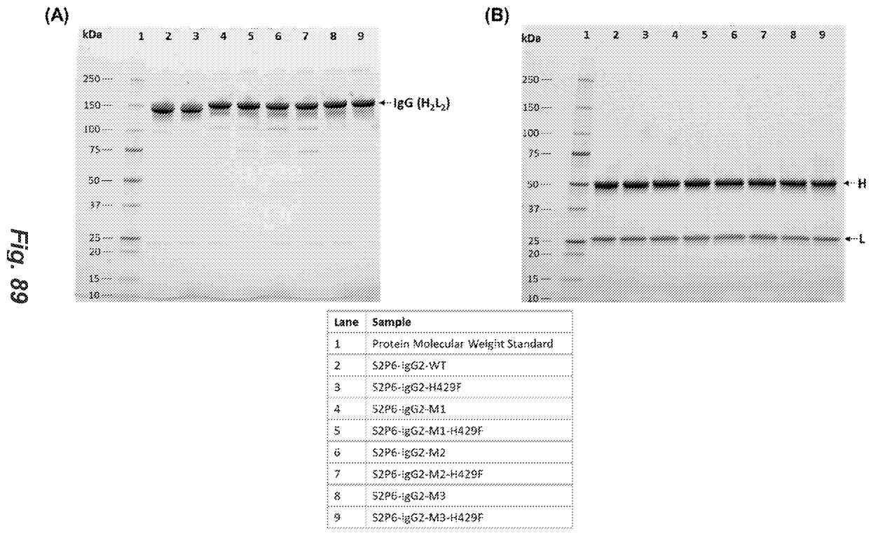

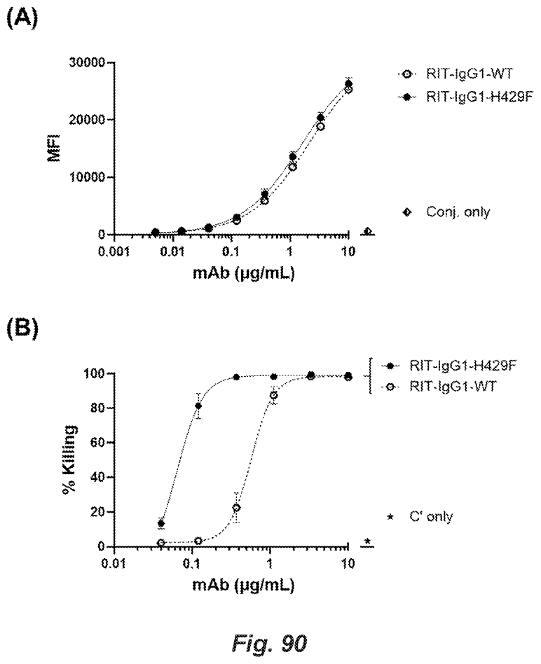

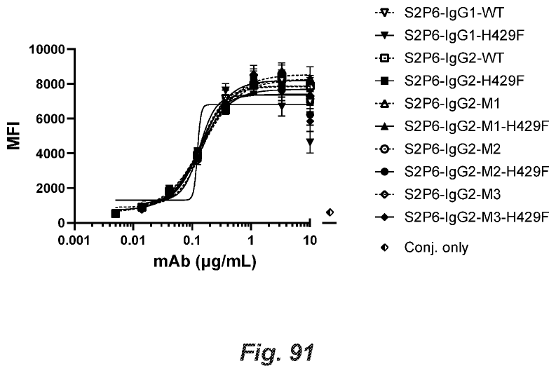

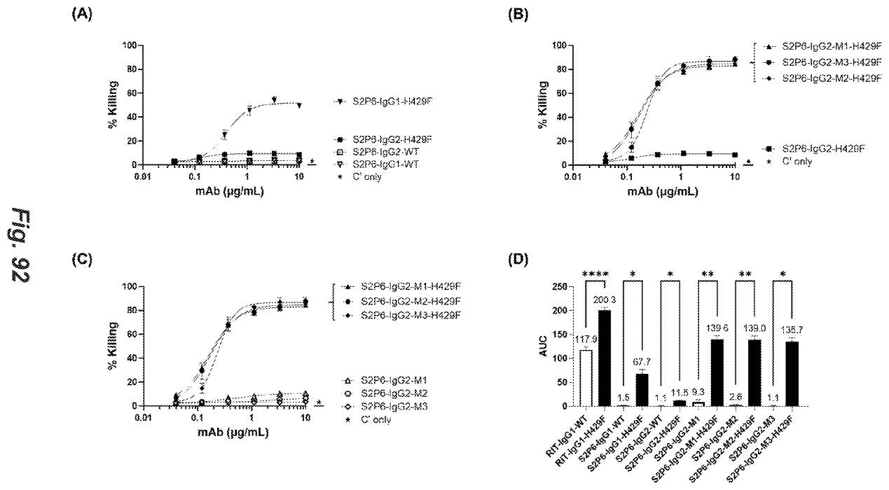

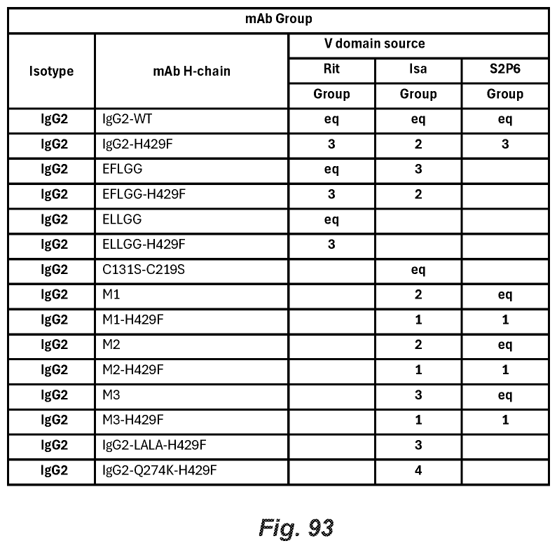

FIG. 53 provides representations of immunoglobulin (antibodies) and immunoglobulin (antibody)-like molecules showing the modular nature of the antibody:
(A) Left panel: The definition of the immunoglobulin molecule chains and components are as indicated and as also defined in the left panel of FIG. 2.
Middle panel: Provides one example of an Ab-like fusion protein, showing that the modular nature of immunoglobulins allows flexibility in the production of Ab-like molecules (as provided in FIG. 2). In the particular depicted Ab-like fusion protein, the target recognition structure (shown as X1) is the same in all chains, as is the case of the Ab-like molecule described in Example 14 (where an EflACE2 polypeptide is separately linked to both the H chain at the CH1 domain and the L chain constant domain, enabling assembly into an H₂L₂ Ab-like fusion protein).
Right panel: Depicts possible Ab-like fusion proteins comprising fusions to different target recognition structures (or enzymes and/or reporter molecules) in any combination of specificities (e.g. “X1 X1 X1 X1”, “X1 X1 X1 X2”, “X1 X1 X2 X2, “X1 X1 X2 X3”, “X1 X2 X3 X4”; where “X1”, “X2”, “X3” and “X4” represent different target recognition structures (or enzymes or reporter molecules));
(B) SDS-PAGE analysis of EflACE2-Ab-like-WT fusion protein eluted from Protein A affinity matrix with 0.4 M arginine (pH 4) demonstrating that a fully disulfide-linked molecule consistent with an H₂L₂ Ab-like configuration was achieved (lane 1). This molecule comprises an EflACE2 polypeptide fusion to the immunoglobulin constant heavy chain (EflACE2-CH) which self assembles with an equivalent EflACE2 polypeptide fused to the immunoglobulin light chain constant domain (EflACE2-CL). Upon reduction with dithiothreitol, these two chains (i.e. EflACE2-CH (ACE2-CH) and EflACE2-CL (ACE2-CL); lane 2) are resolved separately.
(C, D) Provide results which show that the EflACE2-Ab-like-H429F fusion protein (H429F) strongly directs CDC of Ramos-S target cells, whereas a corresponding fusion protein with H chains of wild type sequence (WT) was ineffective. CDC of the opsonised Ramos-S cells was determined using the presence of a 1/3 dilution of normal human serum as a source of complement. This potent CDC mediated by the EflACE2-Ab-like-H429F fusion protein was not attributable to different levels of opsonisation compared to the EflACE2-Ab-like-WT fusion protein, as the binding of these to Ramos cells expressing SARS-CoV-2 spike were comparable for both proteins. Binding was determined using flow cytometric analysis using an anti-IgG secondary reagent labelled with goat anti-hIgG Fc FITC. MFI=median fluorescent intensity; background binding of the anti-Ig fluorescent conjugate shown as (conj only);

FIG. 54 shows the results of purification of the EflACE2-Ab-like-Fc-H429F fusion protein using Protein A and elution with arginine. (A) Protein A chromatography using Hitrap™ Protein A column with gradient elution from 30 mM arginine (pH 4) to 35% of 130 mM arginine (pH 4); (B) Size-exclusion chromatography (SEC) of the pooled and concentrated Protein A fractions containing EflACE2-Ab-like-Fc-H429F using a Superose 6 Increase 10/300 column with oligomeric material indicated (HMW); and (C) SDS-PAGE analysis of the pooled Protein A eluate and the pooled SEC monomeric fractions under non-reducing (without DTT, dithiothreitol) and reducing (with DTT) conditions;

FIG. 55 provides results showing the survival of Colo205 colorectal cells in the presence of purified DR5-specific mAbs comprising IgG1 H chains with the single H429F mutation (HF) or the L234A, L235A and H429F (LA/LA/HF) mutations. The mAbs were titrated individually or mixed and titrated (BDR5-1LA/LA/HF+TDR5-1LA/LA/HF) and compared to a mixture of BDR5-1HF+TDR5-1HF wherein both mAbs in the mixture comprised IgG1 H chains with only the H429F modification; Colo205 cells, 10,000 cells per well of 96-well plate, were cultured in the presence of serial two-fold dilutions of the indicated mAbs. The individual mAbs were titrated from 1 μg/ml. For the titration of the mixtures, the starting concentration of 1 μg/ml was comprised of 0.5 μg/ml of each mAb in the mixture. Cell viability was quantitated using a CCK8 colorimetric cell viability assay, where absorbance (Abs450 nm) of cell culture supernatant is a measure of cell survival and viability. Maximum cell viability was determined by the culture of cells in the absence of antibody (no Ab) and maximum death determined by culture of cells with sodium dodecyl sulphate (SD S). Survival is represented by: [Abs450 nm of mAb or control treatment determined experimentally determined Abs450 nm −background Abs450 of cell culture medium];

FIG. 56 illustrates a sequence comparison of the hinges and constant domains of human immunoglobulin heavy (H) chain sequences and depicts the domain-based structure of the H chain (i.e. CH1-Hinge-CH2-CH3; Numbering of the amino acid positions is shown vertically and follows the Eu numbering and thus: CH1 domain, amino acids 118 to 215; hinge amino acids 216 to 230; CH2 domain amino acids 231 to 340 including the lower hinge positions; and CH3 domain amino acids 341 to 447). The other IgG subclasses IgG2, IgG4, have a corresponding domain structure with amino acid sequence homology. Amino acid sequences were derived from translation of open reading frames of sequences from the European Nucleotide Archive (https://www.ebi.ac.uk/ena/browser). Alignments were performed using Clustal except for the hinges which were aligned manually; gaps represented by a dot, may be present in the aligned sequences for the purpose of optimal sequence alignment; resides 231-237 inclusive, comprise the structural lower hinge of the IgG subclasses but are encoded by the CH2 exon and may also be known in the literature as the hinge proximal region of CH2. Accession numbers of the H chain sequences are: IgG1 H chain, J00228-IGHG1; IgG4 H chain, K01316-IGHG; IgG2 H chain, J00230-IGHG2;

FIG. 57 provides the results of an ELISA showing the capacity of IgG antibodies comprising Fc region components to fix complement as measured by the binding of human C1q. Serial dilutions of mAbs, commencing at 4 μg/ml, were titrated on TNP-BSA antigen adsorbed to the wells of ELISA plates and the binding of human C1q determined and their C1q binding curves are shown. All mAbs showed similar antigen binding activity (not shown); the mAbs recognised TNP hapten and comprised unmodified wild-type (WT) heavy chains of the human IgG1 subclass (TNP-IgG1-WT) or comprised unmodified wild-type (WT) heavy chains of the human IgG2 subclass (TNP-IgG2-WT) or comprised heavy chains of the human IgG2 subclass which had been mutated and are shown as mAb TNP2x4, mAb TNP2x5, mAb TNP2x15 and mAb TNP2x17. O.D.=O.D. (450 nm);

FIG. 58 provides the results of an ELISA showing the capacity of IgG antibodies comprising Fc region components to fix complement as measured by the binding of human C1q. Serial dilutions of mAbs, commencing at 4 μg/ml, were titrated on TNP-BSA antigen adsorbed to the wells of ELISA plates and the binding of human C1q determined and their C1q binding curves are shown. All mAbs showed similar antigen binding activity (not shown); the mAbs tested recognised TNP and comprised unmodified wild-type (WT) heavy chains the human IgG1 subclass, mAb TNP-IgG1-WT, or comprised unmodified wild-type (WT) heavy chains of the human IgG2 subclass (TNP-IgG2-WT) or comprised mutated heavy chains of the human IgG1 subclass and are shown (A) as TNP1x32, and in (B) mAb TNP1x1, mAb TNP1x2, mAb TNP1x3, mAb TNP1x5, mAb TNP1x9, mAb TNP1x10, mAb TNP1x12, mAb TNP1x14 and mAb TNP1x15 O.D.=O.D. (450 nm);

FIG. 59 provides the results of an ELISA showing the capacity of IgG antibodies comprising Fc region components to fix complement is measured by the binding of human C1q. Serial dilutions of mAbs, commencing at 4 μg/ml, were titrated on TNP-BSA antigen adsorbed to the wells of ELISA plates and the binding of human C1q determined and their C1q binding curves are shown. All mAbs showed similar antigen binding activity (not shown); the mAbs tested recognised TNP hapten and comprised unmodified wild-type (WT) heavy chains of the human IgG1 subclass, (mAb TNP-IgG1-WT), or comprised unmodified wild-type (WT) heavy chains of the human IgG2 subclass (mAb TNP-IgG2-WT) or comprised mutated heavy chains of the human IgG1 subclass shown as mAb TNP1x16, mAb TNP1x22, mAb TNP1x23, mAb TNP1x24, mAb TNP1x25. O.D.=O.D. (450 nm);

FIG. 60 provides the results of flow cytometric quantitation of binding of SEC purified mAbs to target cell surface antigens. The mAbs detected CD20 and were based on rituximab variable domains and also comprised unmodified wild-type heavy chains of the IgG2 subclass or IgG1 subclass or comprising mutated IgG2 heavy chains carrying one or more mutations. Binding was evaluated by flow cytometry on Ramos lymphoma cells expressing CD20. The data shows that the mAbs comprising IgG2 based H chains bound similarly to one another and to Rit-IgG2-WT and in every case the level of binding of these IgG2 based mAbs was considerably lower than the binding of rituximab comprising unmodified wild-type heavy chains of the IgG1 subclass (Rit-IgG1-WT). HF is the mutation H429F, EG is the E430G mutation, LLGG is the PVA(233-236)ELLG mutation, FLGG is the PVA(233-236)EFLG mutation;

FIG. 61 provides results showing that H429F modification of the mutants provides superior CDC to mAbs carrying H chain mutation without Stellabody. Complement-dependent cytotoxicity (CDC) of target cells was determined using Daudi lymphoma cells opsonised with the mAbs at the indicated concentrations and normal human serum diluted 1/3 as the source of complement. CDC (% killing) was determined by flow cytometry using Zombie Green. The control background CDC of complement in the absence of mAb (no mAb C′ only) is shown. Whereas Rit-IgG2-WT comprising unmodified wild-type heavy chains failed to mediate detectable CDC, all mAbs that comprised specifically mutated IgG2 H chains mediated detectable CDC. (A) CDC mediated by Rit-IgG2-LLGG or Rit-IgG2-FLGG was superior in CDC compared to the unmodified Rit-IgG2-WT which failed to mediate CDC. (B) CDC mediated by mAb Rit-IgG2-FLGG HF, Rit-IgG2-LLGG HF, Rit-IgG2-FLGG-EG, Rit-IgG2-LLGG EG, Rit-IgG2-HF, Rit-IgG2-LLGG or Rit-IgG2-FLGG was superior compared to CDC by the Rit-IgG2-WT mAb which comprised unmodified H chains and which failed to mediate CDC. The background lysis control (no mAb C′ only) i.e. CDC in the presence of complement (serum) but without mAb is shown in both panels. HF is the mutation H429F, EG is the E430G mutation, LLGG is the PVA(233-236)ELLG mutation, FLGG is the PVA(233-236)EFLG mutation;

FIG. 62 provides the results of a competitive binding assay for anti-CD20 rituximab mAbs on Daudi cells. The mAbs were serially 2-fold titrated from a starting concentration of 20 μg/mL and competition with biotinylated anti-CD20 IgG1-WT (RIT-IgG1-WT-biotin) for binding to the cell surface was measured by flow cytometry: (A) Comparison of RIT-IgG1-WT and RIT-IgG2-WT shows that IgG1 is a better competitor than IgG2 despite having the same VL and VH domains. A control for non-specific effects used each mAb titrated in the absence of the biotinylated anti-CD20 IgG1-WT antibody followed by incubation with the fluorescent conjugate (streptavidin-FITC) (mean±SEM, n=4); (B) Comparison of the IgG1, IgG2, IgG3 and IgG4 human antibody subclasses showing that the IgG2 subclass uniquely demonstrates a reduced capacity to bind to the CD20 antigen (mean±SEM, n=2). In all panels, the maximum binding signal of biotinylated anti-CD20 IgG1-WT in the absence of any competitor and the non-specific binding of streptavidin-FITC alone (conj. only control) is shown. MFI=Median Fluorescence Intensity;

FIG. 63 provides the binding profiles of anti-CD20 rituximab mAbs on target Ramos cells. The binding of mAbs to Ramos cells was detected using goat anti-human IgG Fc FITC and flow cytometry (A) RIT-IgG1 and RIT-IgG2 comprising a WT or a H429F mutant heavy chain, suggesting that rituximab formatted as an IgG2 displays reduced binding to target cells. MAbs were serially 2-fold titrated from 2 μg/mL (mean±SEM, n=2); (B) RIT-IgG2 mAbs comprising the lower hinge residues of IgG4 (RIT-IgG2-EFLGG), the lower hinge residues of IgG1 (RIT-IgG2-ELLGG), the H429F mutation in the CH3 domain (RIT-IgG2-H429F), or a combination of lower hinge and H429F mutations (RIT-IgG2-EFLGG-H429F and RIT-IgG2-ELLGG-H429F). MAbs were serially 2-fold titrated from 20 μg/mL (mean±SEM, n=2); The binding profile of IgG1-WT mAb is shown for comparison. The level of non-specific binding of anti-IgG Fc FITC to cells is shown (▴ conj. only); MFI=Median Fluorescence Intensity;

FIG. 64 provides results showing anti-CD38 mAb variants' ability to kill Ramos cells in the presence of serum. Purified mAbs were serially 2-fold titrated and CDC measured by flow cytometry. Normal human serum diluted 1 in 3 was used as the source of complement. (A) Comparison of the cytotoxic effect of RIT-IgG1 and RIT-IgG2 comprising a WT or a H429F mutant heavy chain. MAbs were serially 2-fold titrated from 2 μg/mL (n=1); (B) Comparison of the cytotoxic effect of RIT-IgG2 mAbs comprising the lower hinge residues of IgG4 (RIT-IgG2-EFLGG), the lower hinge residues of IgG1 (RIT-IgG2-ELLGG), the H429F mutation in the CH3 domain (RIT-IgG2-H429F), or a combination of lower hinge and H429F mutations (RIT-IgG2-EFLGG-H429F and RIT-IgG2-ELLGG-H429F). IgG2-WT and IgG1-WT mAbs were included for comparison. MAbs were serially 2-fold titrated from 40 μg/mL (mean±SEM, n=3). Non-specific target cell killing in the absence of mAb (C′ only) is shown as a filled triangle (▴);

FIG. 65 shows representations of the light chain and the M1, M2 and M3 mutant IgG2 H-chains, inclusive of the VH, CH1, upper hinge, core hinge and lower hinge sequences only. Each indicate the disulfide linkages (depicted by a black line) expected in the upper hinge region of each IgG2 mutant mAb (M1, M2, and M3) between the light chain (LC) and heavy chain (HC). The core hinge, CPPC, forms inter-heavy chain disulfides which are not shown. Remaining cysteine residues capable of forming interchain disulfide bonds are shown as black circles, and cysteine residues that have been substituted with a serine are shown as black circles with a white cross. (A) The M1 mutant comprises the heavy chain upper hinge sequence of IgG2 and a cysteine-to-serine substitution at positions 131 (C131S) in the CH1 domain and 220 (C220S) and is thus expected to form a disulfide bond between the cysteine at position 214 in the light chain (C214) and the cysteine at position 219 (C219) in the upper hinge region of the heavy chain. (B) The M2 mutant comprises the heavy chain upper hinge sequence of IgG2 and a cysteine-to-serine substitution at positions 131 (C131S) and 219 (C219S) and is thus expected to form a disulfide bond between the cysteine at position 214 in the light chain (C214) and the cysteine at position 220 (C220) in the upper hinge region of the heavy chain. (C) The M3 mutant comprises the heavy chain upper hinge sequence of IgG1 (EPKSCDKTHT) and a cysteine-to-serine substitution at positions 131 (C131S) and is thus expected to form a disulfide bond between the cysteine at position 214 in the light chain (C214) and the cysteine at position 220 (C220) in the upper hinge region of the heavy chain. As this mutant has the upper hinge sequence of IgG1, it has 2 additional amino acids;

FIG. 66 provides size exclusion chromatography (SEC) elution profiles of the various isatuximab IgG1 and IgG2 mAbs as shown in each panel. Protein A affinity purified IgG was further purified by SEC at pH 7.2; (A) Isa-IgG1-WT, (B) Isa-IgG2-WT (C) Isa-IgG2-M1, (D) Isa-IgG2-M2, (E) Isa-IgG2-M3, (F) Isa-IgG2-C131S-C219S, and (G) Isa-IgG2-EFLGG are shown, each consisting of a single IgG peak with minimal oligomeric species;

FIG. 67 shows the results of (A) non-reduced SDS-PAGE analysis of SEC purified mAbs, showing that each migrated at the expected ~150 kDa mass of IgG (H2L2). (B) Purified mAbs were also reduced in dithiothreitol and examined by SDS-PAGE analysis, showing that each mAb resolved to its ~50 kDa heavy (H) chain and ~25 kDa light (L) chain species. M=molecular weight marker;

FIG. 68 provides the results of flow cytometric quantitation of the binding of anti-CD38 mAbs to Raji cells. The mAbs were serially 3-fold titrated from 5 μg/mL and the binding to Raji cells was detected by flow cytometry using a goat (Fab′)₂ anti-human-IgG Fc FITC secondary reagent. (A) Isa-IgG1-WT and Isa-IgG2-WT, suggesting that isatuximab formatted as an IgG2 has reduced binding to target cells, (B) Isa-IgG2-WT and hinge-modified IgG2 mutants (Isa-IgG1-M1, Isa-IgG2-M2, and Isa-IgG2-M3), (C) Isa-IgG2-WT, Isa-IgG2-C131S-C219S and Isa-IgG2-EFLG, showing modest effects on binding by either the upper hinge or lower hinge mutations in isolation. In all panels, the level of non-specific binding of goat anti-human-IgG Fc FITC to cells is shown (▴ conj. only); MFI=Median Fluorescence Intensity. Mean±SEM; n=4. P values were determined comparing to Isa-IgG2-WT by 2-way ANOVA for main column effect; and for B, C used Dunnett's multiple comparisons test;

FIG. 69 provides the results of the binding of anti-CD38 isatuximab mAbs to Raji cells by a flow cytometric competitive binding assay. The mAbs were serially 3-fold titrated and used to compete with biotinylated Isa-IgG1-WT for binding. RIT-IgG1-WT was used as a negative control that does not compete with biotinylated Isa-IgG1-WT for binding. (A) Isa-IgG1-WT and Isa-IgG2-WT, shows that the IgG2-WT displays reduced binding to target cells (mean±SEM, n=6); (B) Isa-IgG2-WT, Isa-IgG1-M1, Isa-IgG2-M2, and Isa-IgG2-M3, showing hinge-modification enhances binding and competition (mean±SEM, n=6); (C) Isa-IgG2-WT, Isa-IgG2-C131S-C219S and Isa-IgG2-EFLGG, showing no effect on binding by either the upper hinge or lower hinge mutations in isolation. In all panels, the level of non-specific binding of fluorescent conjugate (streptavidin-FITC) to cells is shown (▴ conj only) and the maximum binding of biotinylated Isa-IgG1-WT (i.e. in the absence of a competitor mAb) is shown (Isa-IgG1-WT-biotin); MFI=Median Fluorescence Intensity. P values were determined comparing to Isa-IgG2-WT by 2-way ANOVA for main column effect and Dunnett's multiple comparisons test;

FIG. 70 provides results showing anti-CD38 mAbs mediate the killing of Raji cells in the presence of serum. Purified mAbs were serially 3-fold titrated and CDC measured by flow cytometry. Normal human serum diluted 1 in 3 was used as the source of complement. (A) Isa-IgG1-WT and Isa-IgG2-WT (mean±SEM, n=6), (B) Isa-IgG2-WT, Isa-IgG1-M1, Isa-IgG2-M2, and Isa-IgG2-M3 (mean±SEM, n=6), (C) Isa-IgG2-WT, Isa-IgG2-C131S-C219S, Isa-IgG2-EFLGG (mean±SEM, n=3). A, B, C) P values determined comparing to Isa-IgG2-WT by 2-way ANOVA by main column effect using B, C) Dunnett's multiple comparisons test; (D) The cell killing in the presence each mAb at the highest concentration tested (5 μg/mL) was measured with (+C′) and without (−C′) human serum as a source of complement (mean±SEM, n=4);

FIG. 71 provides results showing C1q binding, as the C1 complex, to opsonised Ramos cells. Ramos cells were opsonised with 5 μg/mL of anti-CD38 mAbs, IgG-WT and IgG2-mutants, followed by normal human serum (NHS; diluted 1 in 3 in DPBS+BSA) as a source of C1. The binding of C1 was detected with an anti-C1q rabbit polyclonal antibody followed by a fluorescently labelled anti-rabbit-Fc probe. Grey bars indicate C1 binding to mAb opsonized cells, and black bars indicate the level of background fluorescence from mAb opsonized cells in the absence of C1q. Background binding of C1 to cells without mAb is shown in the ‘no mAb’ control. Mean±SEM, n=4; MFI=median fluorescence intensity;

FIG. 72 provides size exclusion chromatography (SEC) profiles of the various isatuximab IgG1 and IgG2 mAbs generated with and without the H429F mutation. Following Protein A affinity purification, mAbs were further purified by SEC at pH 7.2. The SEC elution profiles of (A) Isa-IgG1-WT and Isa-IgG1-H429F, (B) Isa-IgG2-WT and Isa-IgG2-H429F, (C) Isa-IgG2-EFLGG and Isa-IgG2-EFLGG-H429F, (D) Isa-IgG2-M1 and Isa-IgG2-M1-H429F, (E) Isa-IgG2-M2 and Isa-IgG2-M2-H429F, and (F) Isa-IgG2-M3 and Isa-IgG2-M3-H429F are shown, each consisting of a single homogenous IgG peak with minimal oligomeric species. Fractions containing monomeric IgG are outlined in a box with a dotted line and were pooled for subsequent analyses of mAb function;

FIG. 73 shows the results of (A) non-reduced SDS-PAGE analysis of SEC purified mAbs as indicated, showing that each migrated predominately at the expected ~150 kDa mass of IgG (H2L2). (B) Samples were also reduced in dithiothreitol and examined by SDS-PAGE, showing that each mAb resolved to its ~50 kDa heavy (H) chain and ~25 kDa light (L) chain species. M=molecular weight marker;

FIG. 74 provides the results of flow cytometric quantitation of the binding of anti-CD38 isatuximab mAb with or without the H429F mutation to Raji cells. MAbs were serially 3-fold titrated and the binding activity was quantified by flow cytometry using a goat anti-human IgG Fc-FITC: (A) Isa-IgG1-WT and Isa-IgG1-H429F (mean±SEM, n=5); (B) Isa-IgG2-WT and Isa-IgG2-H429F (mean±SEM, n=6); (C) Isa-IgG2-EFLGG and Isa-IgG2-EFLGG-H429F (mean±SEM, n=4); (D) Isa-IgG2-M1 and Isa-IgG2-M1-H429F (mean±SEM, n=5); (E) Isa-IgG2-M2 and Isa-IgG2-M2-H429F (mean±SEM, n=5); (F) Isa-IgG2-M3 and Isa-IgG2-M3-H429F (mean±SEM, n=5). In all panels, the level of non-specific binding of the goat anti-human IgG Fc FITC conjugate to cells is shown (▴ conj); MFI=Median Fluorescence Intensity. P values of nonlinear fits were determined by two-way ANOVA by main column effect;

FIG. 75 provides the results of a competitive binding assay comparing the binding of each anti-CD38 isatuximab mAb with its H429F equivalent. The mAbs were serially 3-fold titrated and competition with biotinylated anti-CD38 IgG1-WT (Isa-IgG1-WT-biotin) for binding to the cell surface was measured by flow cytometry: (A) Isa-IgG1-WT and Isa-IgG1-H429F (mean±SEM, n=5); (B) Isa-IgG2-WT and Isa-IgG2-H429F (mean±SEM, n=6); (C) Isa-IgG2-EFLGG and Isa-IgG2-EFLGG-H429F (mean±SEM, n=3); (D) Isa-IgG2-M1 and Isa-IgG2-M1-H429F (mean±SEM, n=5); (E) Isa-IgG2-M2 and Isa-IgG2-M2-H429F (mean±SEM, n=5); (F) Isa-IgG2-M3 and Isa-IgG2-M3-H429F (mean±SEM, n=5). In all panels, the level of non-specific binding of fluorescent conjugate (streptavidin-FITC) to cells is shown (▴ conj only) and the maximum binding of biotinylated Isa-IgG1-WT is shown (Isa-IgG1-WT-biotin). MFI=Median Fluorescence Intensity. P values determined by two-way ANOVA;

FIG. 76 provides results showing CDC mediated by isatuximab mAbs and their H429F-equivalents on Raji cells. SEC purified mAbs were serially 3-fold titrated and CDC potency was determined by flow cytometry using normal human serum diluted 1 in 3 as the source of complement: (A) Isa-IgG1-WT and Isa-IgG1-H429F (mean±SEM, n=5); (B) Isa-IgG2-WT and Isa-IgG2-H429F (mean±SEM, n=5); (C) Isa-IgG2-EFLGG and Isa-IgG2-EFLGG-H429F (mean±SEM, n=3); (D) Isa-IgG2-M1 and Isa-IgG2-M1-H429F (mean±SEM, n=5); (E) Isa-IgG2-M2 and Isa-IgG2-M2-H429F (mean±SEM, n=5); (F) Isa-IgG2-M3 and Isa-IgG2-M3-H429F (mean±SEM, n=5); In all panels, the level of non-specific target cell killing in the absence of mAb (C′ only) is shown as a filled triangle (▴). P values determined by two-way ANOVA testing for main column effect;

FIG. 77 provides results showing the killing of Raji target cells in the presence of each mAb at the highest concentration tested (5 μg/mL) with (+C′) and without (−C′) human serum as a source of complement, revealing that the observed CDC activity was dependent on the presence of complement. Plots are shown for Isa-IgG variants: (A) IgG1-WT and IgG1-H429F; (B) IgG2-WT and IgG2-H429F; (C) IgG2-EFLGG and IgG2-EFLGG-H429F; (D) IgG2-M1 and IgG2-M1-H429F; (E) IgG2-M2 and IgG2-M2-H429F; (F) IgG2-M3 and IgG2-M3-H429F. Mean±SEM, n=4. P values determined by pairwise t-test with Holm's correction for multiple comparisons. *p<0.05; ns=not significant;

FIG. 78 provides results showing that H429F mutation enhances C1 binding to Ramos cells opsonised with anti-CD38 IgG1-WT or IgG2-WT or the indicated modified IgG2 variants. Ramos cells were opsonised with 5 μg/mL of anti-CD38 mAb before normal human serum (diluted 1 in 3 in DPBS+BSA) was added as a source of C1. The binding of C1 was detected with an anti-C1q rabbit polyclonal antibody followed by a fluorescently labelled anti-rabbit-Fc donkey antiserum and is represented as median fluorescence intensity (MFI). Grey bars indicate C1 binding to mAb opsonized cells in the presence of serum, and black bars indicate the level of background fluorescence from mAb opsonized cells in the absence of serum C1 (mean±SEM, n=4). Background binding of C1 to cells without mAb is shown in the ‘no mAb’ control;

FIG. 79 provides size exclusion chromatography (SEC) profiles of the various isatuximab IgG1 and IgG2 mAbs with the indicated mutations. Following Protein A affinity purification, mAbs were further purified by SEC at pH 7.2. The SEC elution profiles of profiles are shown for Isa-IgG variants; (A) IgG1-H429F, (B) IgG1-LALA, (C) IgG1-LALA-H429F, (D) IgG1-K274Q (E) IgG1-K274Q-H429F, (F) IgG1-H268E-A327D-H429F, (G) IgG2-LALA-H429F, and (H) IgG2-Q274K-H429F, each consisting of a single homogenous IgG peak with minimal oligomeric species. Fractions containing monomeric IgG are outlined in a box with a dotted line and were pooled for subsequent analyses of mAb function;

FIG. 80 shows the results of (A) non-reduced SDS-PAGE analysis of SEC purified mAbs, showing that each migrated predominately at the expected ~150 kDa mass of IgG (H2L2). (B) Samples were also reduced in dithiothreitol and examined by SDS-PAGE, showing that each mAb resolved to its ~50 kDa heavy (H) chain and ~25 kDa light (L) chain species;

FIG. 81 provides the results of mAb binding assays with Raji cells, testing the effects of various mutations in the CH2 of anti-CD38 isatuximab IgG1 and IgG2 mAbs with, or without, H429F modification. The mAbs were serially 3-fold titrated and the binding was quantified by flow cytometry using a goat anti-human IgG Fc-FITC secondary reagent. Binding profiles are shown for Isa-IgG variants: (A) IgG1-WT, IgG1-H429F, IgG1-LALA and IgG1-LALA-H429F; (B) IgG1-WT, IgG1-H429F, IgG1-K274Q, IgG1-K274Q-H429F and IgG1-H268E-A327D-H429F; (C) IgG2-WT, IgG2-H429F, IgG2-LALA-H429F and IgG2-Q274K-H429F. Note that in panel B the IgG1-WT and IgG1-H429F curves are as in panel A and are included for the sake of comparison. The level of non-specific binding of fluorescent conjugate to cells is shown (▴ conj); MFI=Median Fluorescence Intensity. Mean±SEM, n=3. P values were determined by 2-way ANOVA testing for main column effect with comparison to IgG-WT by Dunnett's multiple comparisons test;

FIG. 82 provides the results of competitive binding assays with Raji cells, testing the effects of various mutations in the CH2 of anti-CD38 isatuximab IgG1 and IgG2 mAbs with, or without, H429F modification. The mAbs were serially 3-fold titrated and binding activity of competing biotinylated isatuximab IgG1-WT was determined by flow cytometry. Isa-IgG competitors were: (A) IgG1-WT, IgG1-H429F, IgG1-LALA and IgG1-LALA-H429F; (B) IgG1-WT, IgG1-H429F, IgG1-K274Q, IgG1-K274Q-H429F and IgG1-H268E-A327D-H429F; (C) IgG2-WT, IgG2-H429F, IgG2-LALA-H429F and IgG2-Q274K-H429F. Note that in panel B the IgG1-WT and IgG1-H429F binding curves are as in panel A and are included for the sake of comparison. P values were determined by 2-way ANOVA testing for main column effect with comparison to IgG-WT by Dunnett's multiple comparisons test. In all panels, the level of non-specific binding of fluorescent conjugate (streptavidin-FITC) to cells is shown (▴ conj only) and the maximum binding of biotinylated Isa-IgG1-WT is shown (Isa-IgG1-WT-biotin); MFI=Median Fluorescence Intensity. Mean±SEM, n=2;

FIG. 83 provides the results of cytotoxicity assays, testing the effects of various mutations in the CH2 of anti-CD38 Isa-IgG1 and IgG2 mAbs with, or without, H429F modification, on the killing of Raji cells in the presence of serum. Isa-IgG variants were: (A) IgG1-WT, IgG1-H429F, IgG1-LALA and IgG1-LALA-H429F; (B) IgG1-WT, IgG1-H429F, IgG1-K274Q, IgG1-K274Q-H429F and IgG1-H268E-A327D-H429F; (C) IgG2-WT, IgG2-H429F, IgG2-LALA-H429F and IgG2-Q274K-H429F. Note that in panel B the same binding curves for IgG1-WT and IgG1-H429F are included as in panel A for the sake of comparison. P values were determined by 2-way ANOVA testing for main column effect with comparison to IgG-WT by Dunnett's multiple comparisons test. In all panels, mutant mAbs are compared to the relevant unmodified counterpart to determine the impact of the mutation, and the level of non-specific target cell killing in the absence of mAb (C′ only) is shown as a filled triangle (▴). Mean±SEM, n=3;

FIG. 84 provides results showing the killing of Raji target cells in the presence of each mAb at the highest concentration tested (5 μg/mL) with (+C′) and without (−C′) human serum as a source of complement, revealing that the observed CDC activity was dependent on the presence of complement. The Isa-IgG variants were; (A) IgG1-WT, IgG1-H429F, IgG1-LALA and IgG1-LALA-H429F; (B) IgG1-WT, IgG1-H429F, IgG1-K274Q, IgG1-K274Q-H429F and IgG1-H268E-A327D-H429F; (C) IgG2-WT, IgG2-H429F, IgG2-LALA-H429F and IgG2-Q274K-H429F and each experiment included a no mAb control for non-specific cytotoxicity. Note that in panel B the same data for IgG1-WT and IgG1-H429F are included as in panel A for the sake of comparison. Mean±SEM, n=4;

FIG. 85 provides results showing purified C1q binding on target Ramos cells opsonised with anti-CD38 mAbs. Ramos cells were opsonised with 5 μg/mL of anti-CD38 mAb before 10 μg/mL of purified human C1q was added. The binding of C1q was detected with an anti-C1q rabbit polyclonal antibody followed by a fluorescently labelled anti-rabbit-Fc probe. Isa-IgG variants were; (A) IgG1-WT, IgG1-H429F, IgG1-LALA and IgG1-LALA-H429F; (B) IgG1-WT, IgG1-H429F, IgG1-K274Q, IgG1-K274Q-H429F and IgG1-H268E-A327D-H429F; (C) IgG2-WT, IgG2-H429F, IgG2-LALA-H429F and IgG2-Q274K-H429F. Note that in panel B the same data for IgG1-WT and IgG1-H429F are included as in panel A for the sake of comparison. Grey bars indicate C1q binding to mAb opsonized cells (•+C1q), and white bars indicate the level of background fluorescence from mAb opsonized cells in the absence of C1q (o-C1q). Background binding of C1q to cells without mAb is shown in the ‘no mAb’ control. (Mean±SD, n=2); MFI=median fluorescence intensity;

FIG. 86 provides size exclusion chromatography (SEC) elution profiles of the S2P6 IgG1 mAbs. The mAbs were first purified from culture supernatants by Protein A affinity chromatography followed by SEC at pH 7.2. The SEC elution profiles of (A) S2P6-IgG1-WT, and (B) S2P6-IgG1-H429F are shown, each consisting of a single homogenous IgG peak of monomers with minimal oligomeric species;

FIG. 87 provides the results SDS-PAGE analysis of SEC purified mAbs, S2P6-IgG1-WT and S2P6-IgG1-H429F. Prior to reduction with dithiothreitol (DTT-) both mAbs migrated predominately at the expected ~150 kDa mass of IgG (H2L2). Following reduction in DTT (DTT+) each mAb resolved to its ~50 kDa heavy (H) chain and ~25 kDa light (L) chain species;

FIG. 88 provides size exclusion chromatography (SEC) elution profiles of the various S2P6 IgG2 mAbs generated. MAbs were purified initially by Protein A affinity purification followed by SEC at pH 7.2 as shown. The SEC elution profiles of (A) S2P6-IgG2-WT, (B) S2P6-IgG2-H429F (C) S2P6-IgG2-M1, (D) S2P6-IgG2-M1-H429F, (E) S2P6-IgG2-M2, (F) S2P6-IgG2-M2-H429F, (G) S2P6-IgG2-M3, and (H) S2P6-IgG2-M3-H429F are shown, each consisting of a single monomeric IgG peak with minimal oligomeric species;

FIG. 89 provides the results of (A) non-reduced SDS-PAGE analysis of SEC purified S2P6 IgG2 mAbs, showing that each predominately migrated at the expected ~150 kDa mass of IgG (H2L2). (B) Samples were also reduced in dithiothreitol and examined by SDS-PAGE analysis, showing that each mAb resolved to its ~50 kDa heavy (H) chain and ~25 kDa light (L) chain species;

FIG. 90 provides results showing antigen binding and cell cytotoxicity of rituximab-based mAb variants. (A) anti-CD20 variants RIT-IgG1-WT and RIT-IgG1-H429F were serially 3-fold titrated from 10 μg/ml and binding to Ramos-S cells was determined by flow cytometry using FITC conjugated F(ab′)₂ fragments of goat anti-human IgG-Fc antiserum (n=4). Non-specific binding was determined by staining cells with FITC goat anti-human IgG-Fc antiserum in the absence of mAbs (Conj. only). Ramos-S cells are Ramos cells that express the Spike protein of SARS-CoV-2 (Wuhan). (B) anti-CD20 rituximab variants kill Ramos-S cell targets in the presence of serum. Purified mAbs were serially 3-fold titrated from 10 μg/mL and CDC measured by flow cytometry (n=3). Normal human serum diluted 1 in 3 was used as the source of complement. The concentration dependent cytotoxic effects of RIT-IgG1-WT and RIT-IgG1-H429F are shown. Non-specific target cell killing in the absence of mAb (C′ only) is shown as a filled star (★);

FIG. 91 provides graphical representation of binding of SARS-CoV-2 Spike-specific S2P6-based mAbs to Ramos-S cells. The S2P6 mAb comprised unmodified IgG1, IgG2 H chains or mutated H chain variants as indicated and were serially 3-fold titrated from 10 μg/ml. Their binding to Ramos-S cells was determined by flow cytometry using FITC conjugated F(ab′)₂ fragments of goat anti-human IgG-Fc antiserum (n=4). Non-specific binding was determined by staining cells with FITC goat anti-human IgG-Fc antiserum in the absence of mAbs (Conj. only). All mAbs displayed similar target cell binding;

FIG. 92 provides results showing S2P6 mAb variants killing Ramos-S cell targets in the presence of serum. Ramos-S cells are Ramos cells that express the Spike protein of SARS-CoV-2 (Wuhan). Purified mAbs were serially 3-fold titrated from 10 μg/mL (n=3) and CDC measured by flow cytometry. Normal human serum diluted 1 in 3 was used as the source of complement. The concentration dependent cytotoxic effects of the S2P6 mAbs shown are (A) IgG1-WT, IgG1-H429F, IgG2-WT and IgG2-H429F; (B) IgG2-M1-H429F, IgG2-M2-H429F, IgG2-M3-H429F; (C) IgG2-M1, IgG2-M2, IgG2-M3 and for purposes of comparison the IgG2-M1-H429F, IgG2-M2-H429F, IgG2-M3-H429F curves from panel B are included. (D) Individual experiments from A-C were analysed as plots of CDC killing versus log 10 mAb concentration by calculation of area under the curve (AUC) using the complement only control to set the baseline. Mean±SD, n=3. P values determined by one-way ANOVA with Tukey's multiple comparison test. *p<0.05, **p<0.01, ****p<0.0001; and

FIG. 93 provides groupings of IgG and mutant variants by CDC in comparison to IgG2-WT.

### DETAILED DESCRIPTION

The present disclosure is directed to an immunotherapeutic protein comprising one or more immunoglobulin heavy chain polypeptide comprising one or more of: a constant heavy chain domain 1 (CH1), a constant heavy chain domain 2 (CH2), a constant heavy chain domain 3 (CH3) domain and a hinge region, wherein the one or more polypeptide comprises an amino acid substitution at a position corresponding to H429 of the amino acid sequence of human IgG1, IgG2, IgG3 or IgG4 heavy chain (Eu numbering), and wherein the one or more polypeptide additionally comprises at least one C1q binding modification.

Amino acid numbering used herein is based on the so-called Eu numbering system which relates to the sequence numbering used in the description of the human IgG1 myeloma protein called Eu (Edelman G M et al., Proc Natl Acad Sci USA 63(1):78-85, 1969). In accordance with this system, H429 of IgG1 for example (i.e. histidine at position 429) occurs at position 429 of the Eu sequence. Thus, in any immunoglobulin molecule such as an antibody or fragment thereof or an antibody-like molecule, the amino acid in a given position number relates to, or corresponds to, the corresponding amino acid residue position number in the Eu sequence.

The heavy (H) chain of immunoglobulins such as IgG1 is a modular, multifunctional, but monomeric, polypeptide. The prototypical immunoglobulin or antibody structure (with an H429 mutation may be considered as consisting of a dimeric protein comprising two monomeric heavy (H) chain polypeptides each optionally associated with a light (L) chain and thus occurring in an H₂L₂ format (see FIG. 2). However, other forms of immunoglobulin molecules may be formed, including where one heavy chain (H1) associates with one light (L1) chain in an H₁L₁ format, and other forms where two heavy (H) chains may form dimers in the absence of light (L) chains to produce an H₂ formatted heavy chain complex (FIG. 2). When a light (L) and heavy (H) chain is associated in the H₁L₁ format, the molecule can dimerise in this configuration to yield the typical immunoglobulin structure designated as H₂L₂. This is exemplified by the typical human IgG class of immunoglobulins wherein the two heavy (H) chains are covalently bonded to each other, and a light (L) chain is covalently bonded to each heavy (H) chain.

The dimeric form of H₁L₁ immunoglobulin molecules (i.e. H₂L₂ proteins) is the fundamental structural “unit” of all human Ig classes (i.e. IgG, IgE, IgD, IgA and IgM) and indeed, is also the basis of most mammalian immunoglobulin classes noting that exceptions to this format are known (e.g. camelid immunoglobulins can form a heavy chain dimer without light chains (abbreviated as H₂)). Thus, for example, human immunoglobulin G (IgG) molecules exist physiologically in solution as a single H₂L₂ unit. However, other covalently bonded, higher-order oligomers of the basic H₂L₂ unit do exist in nature and are common, particularly, for IgM and IgA.

For instance, IgM can form covalent pentameric or hexameric rings of the H₂L₂ format with each H₂L₂ unit having disulphide bonds to adjacent H₂L₂ units to form the pentameric oligomer (H₂L₂)₅ or hexameric oligomer (H₂L₂)₆ (Eskeland T and TB Christensen, Scand J Immunol 4(3):217-228, 1975), and notably, it is the hexameric form of IgM which is the most potent effector of the classical complement pathway (which is one of the two major effector systems of the innate immune system initiated by immunoglobulins), activated by the avid binding of the soluble hexameric protein C1q (Eskeland T and T B Christensen, 1975 supra; Randall T D et al., Proc Natl Acad Sci USA 89:962-966, 1992; Hughey C T et al., J Immunol 161:4091-4097, 1998; and Randall T D et al., Eur J Immunol 20:1971-1979, 1990). IgM is also well recognised as the most potent agglutinin by virtue of its covalent oligomerisation which results in the presence of 10-12 specific, and identical, antigen binding variable domains in each IgM pentamer or hexamer. Since an individual antigen recognition structure (e.g. a Fab fragment) has a defined affinity for a target antigenic site or epitope with a strength defined by its monovalent binding to one target structure, the presence of multiple antigen recognition structures in the one molecule (e.g. the 12 antigen binding variable domains of each IgM hexamer) confers stronger or more avid binding of the oligomeric immunoglobulin. Thus, oligovalent binding arises from the combined strength of the individual antigen recognition interactions of the IgM oligomer and its target antigenic epitopes. By comparison, the avidity of IgG (which has two antigen recognition structures (Fabs) per H₂L₂ unit) arises only from the interaction of these two antigen recognition structures.

Immunoglobulins can also be regarded as modular, multifunctional proteins in which the target recognition structure is connected by a flexible linker to a function-activating structure (i.e. the antigen recognition structure provided by the V domain of the Fab is linked by a flexible hinge to the Fc region), and wherein, in the case of a typical immunoglobulin, each H chain and each L chain is comprised of different domains which can be considered structural or functional modules (FIG. 2). An H chain is particularly comprised of an antigen target recognition domain (V_{H} domain) of variable sequence followed by a series of constant domains that are unique to the heavy chain of an Ig class such as, for example, IgG or IgA, and sequence analysis shows that these constant domains are related between immunoglobulin classes (see FIGS. 3 and 4). The V_{H} domain is thus followed by a first constant domain (CH1). This first constant domain is connected by a flexible polypeptide that acts a linker known as the hinge region, to a second constant domain (CH2) which is followed, in turn, by a third constant domain (CH3), and thus a typical H chain consists of discrete structural molecules from the amino terminus (i.e. NH₂-terminus V_{H}-CH1-hinge-CH2-CH3 (FIG. 2)) and is exemplified by the human IgG1 H chain sequence (SEQ ID NOS: 3) and is conserved across all human heavy chains (FIG. 3, FIG. 4) exemplified by human IgG3 (SEQ ID NOS: 4), IgG4 (SEQ ID NO: 5), IgG2 (SEQ ID NOS: 6), IgAQ1 (SEQ ID NOS: 7), IgA2 (SEQ ID NOS: 8) and mammalian immunoglobulins generally. Similarly, a light (L) chain is comprised of a variable antigen target recognition domain (V_{L} domain) followed by a constant domain unique to the light chain class.

It should be noted that the polypeptides that comprise the heavy chains of the IgG subclasses contain a structurally unique segment referred to as the hinge region. The hinge region of the IgG polypeptide is commonly considered to be composed of an upper hinge sequence, core hinge sequence, and a lower hinge sequence. The upper hinge is comprises residues 216-225 inclusive, the core hinge comprises residues 226-230 inclusive and the lower hinge comprises residues 231-237 according to Eu numbering. It should be noted that the amino acid residues which comprise the structural lower hinge of the IgG polypeptide are encoded by the CH2 exon and may also know in the literature as the hinge proximal region of CH2. In this application reference to CH2 includes reference to the lower hinge residues.

In some embodiments, the immunotherapeutic protein is a human immunotherapeutic protein. In some embodiments, the immunotherapeutic protein is a humanised or chimeric immunotherapeutic protein. In some embodiments, the immunotherapeutic protein is a non-human primate immunotherapeutic protein (e.g. monkey). In some embodiments, the immunotherapeutic protein is a rodent (e.g. a mouse immunotherapeutic protein).

In an embodiment, the immunotherapeutic protein comprises one or more of: a constant heavy chain domain 1 (CH1), a constant heavy chain domain 2 (CH2), a constant heavy chain domain 3 (CH3) domain and/or a hinge region. In an embodiment, the immunotherapeutic protein comprises a CH1 domain. In an embodiment, the immunotherapeutic protein comprises a CH2 domain. In an embodiment, the immunotherapeutic protein comprises a CH3 domain. In an embodiment, the immunotherapeutic protein comprises a hinge region. In an embodiment, the immunotherapeutic protein is derived from an IgG1 heavy chain polypeptide. In an embodiment, the immunotherapeutic protein is derived from an IgG2 heavy chain polypeptide. In an embodiment, the immunotherapeutic protein is derived from an IgG3 heavy chain polypeptide. In an embodiment, the immunotherapeutic protein is derived from an IgG4 heavy chain polypeptide.

Given their modular nature, immunoglobulins provide a versatile platform for the creation of a diverse range of immunotherapeutic or diagnostic molecules, including bivalent antibodies such as a classical hybridoma-derived mAb, heavy-chain antibodies comprising dimers of heavy chains (e.g. camelid antibodies; Hamers-Casterman C et al., Nature 363:446, 1993), antibody-like (Ab-like) molecules (including some fusion proteins comprising at least a CH3 domain (or at least a CH4 domain)) and other immunoglobulin derivatives known to those skilled in the art as summarised in the “Periodic Table of Antibodies”; https://absoluteantibody.com/periodic-table-of-antibodies/ #, the entire disclosure of which is herein incorporated by reference). Other specific examples include antibodies and antibody fragments such as single chain Fv (scFv) antibody fragments, asymmetric bi-specific antibodies (WO 2012/058768), strand-exchange engineered domain (SEED or Seed-body) which are asymmetric and bispecific antibody-like molecules (WO2007110205); dual variable domain immunoglobulin (U.S. Pat. No. 7,612,181), knobs-into-holes antibody formats (WO 1998/050431), duobody molecules (WO 2011/131746), IgG-like bispecific antibodies (Shen J et al., J Immunol Methods 318(1-2):65-74, 2007), and fusion proteins comprising an Fc or Fc region component such as scFv-fusions and dual scFv-fusions.

In this specification, a number of terms are used which are well known to those skilled in the art. Nevertheless, for the purposes of clarity, a number of these terms are hereinafter defined.

As used herein, the term “antibody” is to be understood as including (unless specifically indicated as otherwise) polyclonal antibodies (pAb), monoclonal antibodies (mAb), chimeric antibodies, humanised antibodies, antibody mixtures (e.g. recombinant polyclonal antibodies) such as those generated, for example, using methods well known to those skilled in the art for producing multiple antibodies with different specificities from a single host cell line (e.g. Oligoclonics® technology: Merus BV, Utrecht, The Netherlands) or a transgenic animal. Further, it is to be understood that an antibody may be of any immunoglobulin class (i.e. isotype) or allotype. Thus, for example, an antibody as disclosed herein may be of an isotype selected from the well-known immunoglobulin isotypes, or comprise components from more than one isotype (e.g. as may be the case with some chimeric antibody types).

Also, an antibody may comprise “mixed” chains; for example, an antibody comprised of a H₂ or H₂L₂ unit where the H chains are different (i.e. H^{x} and H^{y} (H^{x}H^{y} or L^{x}H^{x}H^{y}L^{y}).

The term “antibody-like molecule” (Ab-like molecule) is to be understood as referring to proteins which are not antibodies and minimally comprise a target recognition structure (e.g. a receptor or ligand such as an antigen recognition sequence (e.g. a variable (V) domain or complementarity-determining region (CDR) of an immunoglobulin) linked to at least a CH3 domain (or a CH4 domain) of an immunoglobulin heavy (H) chain (providing the molecule with an “H-like” chain), and includes, for example, receptor fusion proteins comprising at least a CH3 domain (or at least a CH4 domain) of an H chain. Examples of Ab-like molecules include those mentioned above (such as those depicted in FIG. 2, FIG. 53A and/or The Periodic Table of Antibodies). In some examples, an H-like chain comprising at least a CH3 domain is considered to comprise an Fc region component and may be, for example, a complete Fc region, or merely a CH3 domain, or a CH2-CH3 or CH3-CH2 component. In other examples, such an H-like chain may further comprise, for example, an immunoglobulin hinge sequence. The H-like chain may enable dimerisation, such that an antibody-like molecule may be provided, for example, in a homodimeric or heterodimeric form. As depicted in FIG. 53, in some embodiments, an Ab-like molecule may comprise a H₂L₂ format and may comprise, for example, fusion proteins comprising the same or different target recognition structures (or enzymes and/or reporter molecules) such as “X1 X1 X1 X1”, “X1 X1 X1 X2”, “X1 X1 X2 X2”, “X1 X1 X2 X3” or “X1 X2 X3 X4”, where X1, X2, X3 and X4 represent different target recognition structures (or enzymes or reporter molecules).

The term “fusion protein” is well known to those skilled in the art and refers to a protein expressed from a DNA construct comprising two or more open reading frames in a desired order, such that the protein may be regarded as a hybrid or chimeric protein. In some simple examples, the fusion protein comprises a protein (or fragment) of interest linked (“fused”) to the amino-terminus (N-terminus) or carboxyl-terminus (C-terminus) of a partner polypeptide such as a carrier protein (e.g. human serum albumin, HSA). As used herein, the term “fusion protein” will also be understood as referring to certain types of an antibody-like molecule such as a fusion protein comprising a target recognition structure (e.g. a receptor or ligand such as an antigen recognition sequence (e.g. a variable (V) domain or complementarity-determining region (CDR) of an immunoglobulin)) linked to at least a CH3 domain (or at least a CH4 domain) of an immunoglobulin heavy (H) chain (see examples shown in FIG. 2). Such fusion proteins may comprise, for example, mixed Fc regions (e.g. in a manner similar to that described above for antibodies (e.g. bispecific antibodies) and antibody-like molecules such that the fusion protein comprises, for example, an Fc fragment wherein the Fc regions (chains) are different (e.g. Fc1x and Fc2y), and wherein each of the Fc regions may optionally be linked to different target recognition structures (e.g. ligand 1 and ligand 2). Those skilled in the art will understand that the target or antigen recognition structure may be linked to a polypeptide comprising at least a CH3 domain (or an equivalent CH4 domain of an IgE or IgM) that may, for example, comprise an entire H-chain or comprise an H-like chain which may comprise, for example, a complete Fc region, or merely a CH3 domain, or a CH2-CH3 or a CH3-CH2 component. In other examples, such an H-like chain may further comprise, for example, an immunoglobulin hinge sequence and/or a CH1 domain. A CH1 domain may, for example, provide a suitable site for linking the target or antigen recognition structure.

The term “Fc fragment” (Fc or Fc portion), as used herein, refers to a dimer formed by covalent and/or non-covalent interactions between parts of the immunoglobulin heavy (H) chain (i.e. a dimer formed between two Fc regions of the H chain (which each comprise CH2 and CH3 domains of a heavy chain and optionally a hinge sequence)), and which is responsible for much of the activation of the immune effector functions of immunoglobulins: particularly the cell-based effector responses initiated by antibodies and cell surface-located Fc receptors such as killing of target cells by antibody dependent cell-mediated cytotoxicity (ADCC) or antibody-dependent cell-mediated phagocytosis (ADCP) or trogocytosis, and which also leads to the modulation and inhibition of the activity of innate and adaptive immune cells by inhibitory Fc receptors; and the effector responses of the innate immune system initiated by antibodies through activation of the the classical pathway of complement system, a cascade of proteins found in blood or biological fluids that is important in the destruction of pathogens and involves the killing of targets by direct lysis through complement-dependent cytotoxicity (CDC) and/or by killing via phagocytosis of targets through specific receptors of the components of complement (C′ADCP). The Fc fragment also provides a site of association between immunoglobulin molecules permitting the assembly (self-association) of immunoglobulin molecules into higher order oligomers (e.g. through covalent bonding between domains in the Fc region of the H chain of one immunoglobulin with an Fc region of an H chain of an adjacent immunoglobulin (such as seen in, for example, pentameric and hexameric forms of IgM) and non-covalent self-association such as that seen in IgG when bound to antigen and which, among other properties, leads to oligomerisation including hexamer formation (Diebolder C A et al., Science 343(6176):1260-1263, 2014) and to certain effector functions).

As used herein, the term “Fc region component” is to be understood as referring to a part of the Fc region of an immunoglobulin heavy (H) chain comprising at least a CH3 domain (or at least a CH4 domain), where H429 is located, but preferably comprising a CH2 and CH3 domain and optionally further comprising an immunoglobulin hinge sequence (which may, in turn, comprise all or a portion of the lower hinge, core hinge and upper hinge sequences), that is capable of forming (e.g. by dimerisation) an Fc fragment or Fc-like fragment. An “Fc-like fragment” is to be understood as referring to an Fc fragment-like structure, but which comprises fragments or components of the Fc region e.g. the CH3 domain (or CH4 domain) alone or a CH3 domain in combination with a CH2 domain and optionally further comprising an immunoglobulin hinge sequence which may, in turn, comprise all or a portion of the lower hinge, core hinge and upper hinge sequences.

As used herein, the term “treating” includes prophylaxis as well as the alleviation of established symptoms of a disease or condition. As such, the act of “treating” a disease or condition therefore includes: (1) preventing or delaying the appearance of clinical symptoms of the disease or condition developing in a subject suffering from, or predisposed to, the disease or condition; (2) inhibiting the disease or condition (i.e. arresting, reducing or delaying the development of the disease or condition or a relapse thereof, in case of a maintenance treatment, or at least one clinical or subclinical symptom thereof); and (3) relieving or attenuating the disease or condition (i.e. causing regression of the disease or condition or at least one of clinical or subclinical symptom thereof).

As used herein, the phrase “manufacture of a medicament” includes the use of one or more immunotherapeutic protein as defined herein directly as the medicament or in any stage of the manufacture of a medicament comprising one or more immunotherapeutic protein as defined herein.

The term “effective amount” is an amount sufficient to effect beneficial or desired clinical results. An effective amount can be administered in one or more administrations. Typically, an effective amount is sufficient for treating a disease or condition or otherwise to palliate, ameliorate, stabilise, reverse, slow or delay the progression of a disease or condition. By way of example only, an effective amount of an immunotherapeutic protein such as a mutant IgG1 antibody may comprise between about 0.1 and about 250 mg/kg body weight per day, more preferably between about 0.1 and about 100 mg/kg body weight per day and, still more preferably between about 0.1 and about 25 mg/kg body weight per day. However, notwithstanding the above, it will be understood by those skilled in the art that an effective amount may vary and depend upon a variety of factors including the age, body weight, sex and/or health of the subject being treated, the activity of the particular immunotherapeutic protein, the metabolic stability and length of action of the particular immunotherapeutic protein, the route and time of administration of the particular immunotherapeutic protein, the rate of excretion of the particular immunotherapeutic protein and the severity of, for example, the disease or condition being treated.

As shown in the Examples provided hereinafter, the combination of a hexamerising mutation and a C1q binding modification have functional advantages. In an embodiment, the hexamerising mutation is a point mutation at the H429 position.

As shown in the Examples provided hereinafter, it has been found that a mutant antibody including a point mutation at the H429 position may confer significant functional changes. For example, an IgG mutant with an H429F substitution shows an enhanced ability to activate complement-dependent cytotoxicity (CDC) in complement assays (i.e. assays of complement function). Also, it has been found that a fusion protein comprising an Fc region component with a point mutation at position 429 (i.e. H429F) fused to an angiotensin-converting enzyme 2 (ACE2) ectodomain (which may act as a “decoy” to block viral interaction and cellular entry of coronaviruses to host cells) may provide an enhanced ability to provide an antiviral effect through CDC of infected cells, while a similar fusion protein with an H429Y substitution displays enhanced virus neutralisation with little or no complement activation (since immunotherapeutic proteins comprising an H429Y modified Fc component shows abrogated FcγR binding and activation, especially with FcγRIIIa). While not wishing to be bound by theory, it is considered that these effects may be due to oligomerisation of the proteins into oligomers through the self-association of the antibodies/fusion proteins either in solution or upon binding to a relevant target molecule (e.g. an antigen to which an antibody mutant is directed) through “on target” oligomerisation.

Further in this regard, it is to be noted that while monomeric IgG1 and IgG3 can deliver CDC, the level of CDC can be regarded as low or “poor” by comparison to that achieved with naturally pentameric or hexameric IgM. Moreover, IgG antibodies such as IgG1 and IgG3, unlike IgM, are also comparatively poor agglutinins by virtue of their bivalency. Thus, by enabling oligomerisation (by possibly enhancing the known weak inherent capacity of the Fc of an immunoglobulin such as IgG to self-associate, especially after the immunoglobulin has bound to an antigen (i.e. “on target” oligomerisation), an immunotherapeutic protein according to the present disclosure may, by forming oligomers, provide a more optimal platform for complement system activation and other functions enhanced by self-association. This oligomerisation may, particularly and/or optimally involve the formation of a hexamer; and such hexamerisation has been visualised in the crystallographic structure of anti-HIV antibody b12 (Saphire E O et al., Science 293:1155-1159, 2001), wherein CH3 residues form an interface between adjacent IgG:IgG molecules, so forming a hexamer that optimally presents binding sites for the six globular head domains of the C1q subunit which initiates the activation of the classical complement pathway.

Moreover, many cellular molecules, including cell surface molecules, require substantial clustering by a ligand (which may be soluble or another cell surface molecule) to induce a signal that consequently induces a cellular response, and thus the dimerisation that will typically be achieved with an antibody, may not be sufficient to induce a signal that leads to a meaningful cellular response. However, it is known that the strength of the signal can be increased by increasing clustering of the target molecule through, for example, approaches involving the addition of other entities that cross-link the ligand (Chenoweth et al., Immunol Cell Biol 98:287-304, 2020), and thus, in the case of an antibody (such as a mAb) bound to its target molecule, such “super cross-clustering” or “hyper-clustering” of the target molecule can be achieved by the use of additional anti-immunoglobulin antibodies that cross-link the mAb that is bound to the target molecule. While not wishing to be bound by theory, it is considered that proteins such as a mutant antibody including an H429F point mutation as mentioned above, through self-association upon binding to a target molecule, may similarly achieve super cross-clustering or hyper-clustering” of the target molecule. Depending upon the nature of the target molecule, such clustering might induce an enhanced signalling response that may lead, for example, to cell proliferation (e.g. where the target molecule is, for example, CD3 or CD28), the stimulation of an inhibitory pathway to inhibit or reduce cell responses (e.g. immune checkpoints; reviewed in Chenoweth et al., supra 2020), or the stimulation of a pathway that induces cell death such as, for example, programmed cell death including apoptosis (see, for example, Ashkenazi A., Nat Rev Drug Discov 7(12):1001-1012, 2008). Stimulation of cellular pathways by mAbs for the development of therapeutic molecules is an approach being used to treat a range of diseases including cancers, inflammation and autoimmune diseases, infections, cardiovascular diseases and others (Ashkenazi, supra 2008).

Thus, in an aspect, the present disclosure provides an immunotherapeutic protein comprising one or more immunoglobulin heavy chain polypeptide comprising an Fc region component comprising at least a CH3 domain (or at least a CH4 domain), wherein said one or more polypeptide includes an amino acid substitution at a position corresponding to H429 of the amino acid sequence of human IgG1, IgG2, IgG3 or IgG4 heavy chain (Eu numbering).

In some embodiments, the immunotherapeutic protein as described herein comprises a dimeric immunotherapeutic protein comprising first and second immunoglobulin heavy chain polypeptides comprising an Fc region component comprising CH2 and CH3 domains (so that the first and second polypeptides may form (e.g. by dimerisation) an Fc fragment or Fc-like fragment), wherein the Fc region component of at least one of said first and second polypeptides comprises an amino acid substitution at a position corresponding to H429 of the amino acid sequence of human IgG1 heavy chain (Eu numbering).

Accordingly, the immunotherapeutic protein of such embodiments may be an immunoglobulin molecule such as, for example, an antibody or an antibody-like molecule such as a dimeric polypeptide comprising a pair of single-chain Fv polypeptides linked via Fc fragments (i.e. scFv-Fc) or a mini-body (i.e. a protein comprising a pair of scFv polypeptides linked via CH3 domains (see the discussion of multivalent scFv-Fc and minibodies in, for example, Olafsen T et al., Generation of Antibody Fragments and Their Derivatives, Antibody Engineering Second edition, pp 69-84, 2010)).

In other embodiments, the immunotherapeutic protein as described herein comprises a partner polypeptide linked to an Fc region component comprising at least a CH3 domain (or at least a CH4 domain), wherein the Fc region component comprises an amino acid substitution at a position corresponding to H429 of the amino acid sequence of human IgG1 heavy chain (Eu numbering).

Accordingly, the immunotherapeutic protein of such embodiments may be provided in the form of a fusion protein or protein conjugate. Those skilled in the art will understand that in a fusion protein, the partner polypeptide will be covalently linked (i.e. “fused”) to the Fc component (i.e. as a fusion partner) via a peptide bond or linker sequence (e.g. a short peptide linker sequence such as an immunoglobulin hinge sequence or a well-known glycine-serine linker such as GGGGS) at the N- or C-terminus of the fusion partner (i.e. Fc component), whereas in a protein conjugate, the partner polypeptide will be covalently or non-covalently linked to the Fc component (i.e. as a conjugate partner) through a chemical linkage such as a disulphide bond (e.g. through one or more cysteine (C) residue) or cross-linker compound such as a homobifunctional cross-linker such as disuccinimidyl suberate (DSS) (e.g. bis(sulfosuccinimidyl)suberate (BS³); Thermo Fisher Scientific, Waltham, MA, United States of America) or disuccinimidyl tartrate (DST) to link amine groups or a heterobifunctional cross-linker such as m-maleimidobenzoyl-N-hydroxysuccinimide ester (MDS) and N-(ε-maleimidocaproloxy) succinimide ester (EMCS), or by other non-covalent bonding such as hydrogen bonding. Where the Fc component is a conjugate partner, the immunotherapeutic protein conjugate may be considered as a cross-linked protein, and the Fc component may be conjugated to the partner polypeptide at the N- or C-terminus, but otherwise at any other suitable site on the partner polypeptide. Alternatively, the Fc component (as a conjugate partner) may be conjugated to the partner polypeptide at the N- or C-terminus of the Fc component or otherwise at any other suitable site on the Fc component (e.g. within CH1 or the upper hinge sequence if these are included in the Fc region component).

The Fc region component (also referred to hereinafter as the “Fc component”) of the immunotherapeutic protein may be derived from one or more immunoglobulin type (e.g. an IgG or IgA) and may comprise a full length (i.e. “complete”) Fc region such as, for example, a heavy (H) chain polypeptide fragment corresponding to that generated by papain digestion (i.e. wherein the polypeptide is cleaved within the upper hinge sequence to generate an Fc region comprising the constant heavy domain 2 (CH2; amino acid A231 to K340 of the human IgG1 heavy chain polypeptide (Eu numbering)), constant heavy domain 3 (CH3; amino acid G341 to G446 or K447 of the human IgG1 heavy chain polypeptide (Eu numbering)) and lower hinge sequence (also known as hinge proximal sequence of CH2; amino acids P232 to P238 (Eu numbering)) and core hinge sequence (amino acids C226 to C229) and similar heavy chain polypeptide fragments that may be prepared through digestion of an immunoglobulin heavy chain polypeptide with plasmin and human neutrophil elastase (NHE). Further examples of suitable Fc region components may comprise fragments of the heavy chain polypeptide which comprise, in addition to the CH2 and CH3 domains and the lower and core hinge sequences, all or part of the upper hinge sequence and constant heavy domain 1 (CH1). On the other hand, other suitable Fc region components may comprise fragments of the heavy chain polypeptide which comprise only the CH3 domain (e.g. amino acid G341 to G446 or K447 of the human IgG1 heavy chain polypeptide (Eu numbering)) or a fragment thereof. In addition, suitable Fc region components may comprise heterogeneous (“hybrid”) CH3 domains such as strand exchange engineered domain (SEED) forms of a CH3 domain comprising fragments derived from the IgG1 CH3 domain and other proteins such as IgA (Davies et al., Prot Eng Des Sel 23(4):195-202, 2009).

In some embodiments, the Fc region component is derived from a human immunoglobulin heavy chain polypeptide (e.g. such as those shown in FIGS. 3 and 4).

In other embodiments, the Fc region component is derived from an IgG heavy chain polypeptide, preferably an IgG1 or IgG3 (e.g. human IgG1 or IgG3) heavy chain polypeptide, and more preferably, an IgG1. In some embodiments, the Fc region is glycosylated. In some embodiments, the Fc region does not comprise an E430G substitution.

The Fc region component comprises an amino acid substitution at a position corresponding to H429 of the amino acid sequence of the human IgG1, IgG2, IgG3 or IgG4 heavy chain polypeptide (Eu numbering). Where the Fc region component is derived from another immunoglobulin type or isotype (or from an immunoglobulin from another species), it will be appreciated that those skilled in the art can readily determine a position corresponding to H429 of the human IgG1 heavy chain polypeptide IgG1 by, for example, routine sequence alignments (e.g. as shown in FIGS. 3 and 4). In some embodiments, the H429 amino acid substitution is an aromatic or cyclic amino acid. In some embodiments, the H429 amino acid substitution is an aromatic amino acid. In some embodiments, the H429 amino acid substitution is a cyclic amino acid. In some embodiments, the H429 amino acid substitution is hydrophobic amino acid. In some embodiments, the H429 amino acid substitution is a very hydrophobic amino acid (e.g. F or Y). In some embodiments, the H429 amino acid substitution has a hydrophobicity of T or greater. In some embodiments, the H429 substitution is not a neutral amino acid (e.g. not glycine). In some embodiments, the H429 substitution is not glycine or leucine. In some embodiments, the H429 substitution is not glycine. In some embodiments, the H429 substitution is not leucine.

Included among the suitable mutations at position 429 are:
H→X, where X is selected from phenylalanine (H429F), glutamate (H429E), glutamine (H429Q), serine (H429S), alanine (H429A), threonine (H429T), tyrosine (H429Y), leucine (H429L), valine (H429V), glycine (H429G), tryptophan (H429W), arginine (H429R) and proline (H429P).

Included among the suitable mutations at position 429 are:
H→X, where X is selected from tyrosine (H429Y), phenylalanine (H429F), tryptophan (H429W), glutamate (H429E), aspartate (H429D), glutamine (H429Q), serine (H429S), asparagine (H429N), and threonine (H429T).

Some preferred amino acid substitutions at the position corresponding to H429 of the amino acid sequence of the human IgG1 heavy chain polypeptide include the H→Y (e.g. H429Y) substitution and the H→F (e.g. H429F) substitution.

In some embodiments, H429 is substituted with phenylalanine (H429F). In some embodiments, H429 is substituted with glutamate (H429E). In some embodiments, H429 is substituted with glutamine (H429Q). In some embodiments, H429 is substituted with serine (H429S). In some embodiments, H429 is substituted with alanine (H429A). In some embodiments, H429 is substituted with threonine (H429T). In some embodiments, H429 is substituted with leucine (H429L). In some embodiments, H429 is substituted with valine (H429V). In some embodiments, H429 is substituted with glycine (H429G). In some embodiments, H429 is substituted with tryptophan (H429W). In some embodiments, H429 is substituted with arginine (H429R). In some embodiments, H429 is substituted with and proline (H429P).

As used herein “C1q binding modification” refers to a modification that increases or decreases C1q binding of an immunotherapeutic protein as described herein. In some embodiments, the modification directly decreases/increases binding of the immunotherapeutic protein to C1q compared to a control immunotherapeutic protein lacking the C1q binding modification. In some embodiments, the modification indirectly decreases/increases binding of the immunotherapeutic protein to C1q compared to a control immunotherapeutic protein lacking the C1q binding modification. For the sake of clarity, the term “C1q binding modification” does not require that the modification itself binds to C1q, however this is not excluded.

In some embodiments, the C1q binding modification is in the CH1 domain of the immunotherapeutic protein. In some embodiments, the C1q binding modification is in the CH2 domain of the immunotherapeutic protein. In some embodiments, the C1q binding modification is in the CH3 domain of the immunotherapeutic protein. In some embodiments, the C1q binding modification is in the hinge region of the immunotherapeutic protein. In some embodiments, the mutation is in the lower hinge. In some embodiments, the mutation is in the upper hinge.

In some embodiments, the C1q binding modification is selected from a modification in C1q group 1, C1q group 2, C1q group 3, C1q group 4 and C1q group 5 as described in Table 6. In some embodiments, the C1q binding modification is a C1q group 1 modification as described in Table 6. In some embodiments, the C1q binding modification is a C1q group 2 modification as described in Table 6. In some embodiments, the C1q binding modification is a C1q group 3 modification as described in Table 6. In some embodiments, the C1q binding modification is a C1q group 4 modification as described in Table 6. In some embodiments, the C1q binding modification is a C1q group 5 modification as described in Table 6.

In some embodiments, the C1q binding modification is selected from a modification in C1q Group 1, C1q Group 2 and C1q Group 3 and wherein the modification increases C1q binding compared to a control IgG2 immunotherapeutic protein and/or a control IgG1 immunotherapeutic protein lacking the C1q binding modification.

In some embodiments, the C1q binding modification is selected from a modification in C1q Group 4 and C1q Group 5 and wherein the modification decreases C1q binding compared to a control IgG2 immunotherapeutic protein and/or a control IgG1 immunotherapeutic protein lacking the C1q binding modification.

In some embodiments, the polypeptide is selected from an IgG1, IgG2, IgG3 or IgG4. In some embodiments, the polypeptide is an IgG1. In some embodiments, the polypeptide is an IgG2. In some embodiments, the polypeptide is an IgG3. In some embodiments, the polypeptide is an IgG4.

In an embodiment, the polypeptide is an IgG1, the C1q binding modification is in the CH1 domain and wherein the modification is a substitution at a position corresponding to S131 of the amino acid sequence of a human IgG1 heavy chain polypeptide substituted with a C (Eu numbering).

In an embodiment, the polypeptide is an IgG1, IgG2, IgG3 or IgG4, the modification is in the hinge region and wherein the modification is a substitution at a position corresponding to 216-225 or 217-225 of a human IgG1, IgG2, IgG3 or IgG4 heavy chain polypeptide (Eu numbering). In an embodiment, the substitution in the hinge region is selected from: RKCCVE217-225 substituted with PKSCDKTHT, EPKSCDKTHT216-225 substituted with ERKCCVE and EPKSCDKTHT216-225 substituted with ESKYGPP (Eu numbering). In an embodiment, the substitution in the hinge region is RKCCVE217-225 substituted with PKSCDKTHT. In an embodiment, the substitution in the hinge region is EPKSCDKTHT216-225 substituted with ERKCCVE. In an embodiment, the substitution in the hinge region is EPKSCDKTHT216-225 substituted with ESKYGPP.

In an embodiment, the polypeptide is an IgG2, the modification is in the CH2 domain and wherein the modification is a substitution at a position corresponding to PVA233-236, L328 or S267 of a human IgG1, IgG2, IgG3 or IgG4 heavy chain polypeptide (Eu numbering). In an embodiment, the substitution in the CH2 domain is selected from: PVA233-236 substituted with EFLG, PVA233-236 substituted with ELLG, and PVA233-236 substituted with EFEG. In an embodiment, the substitution in the CH2 domain is PVA233-236 substituted with EFLG (Eu numbering). In an embodiment, the substitution in the CH2 domain is PVA233-236 substituted with ELLG (Eu numbering).

In an embodiment, the polypeptide is an IgG1, IgG3 and IgG4, the modification is in the CH2 domain and wherein the modification is a substitution at a position corresponding to L235 of a human IgG1, IgG3 or IgG4 heavy chain polypeptide substituted with an E (Eu numbering).

In an embodiment, the polypeptide is an IgG1, IgG2, IgG3 or IgG4 and wherein the modification is in the CH2 domain and wherein the modification is a substitution at a position corresponding to QYN295-297, YNS296-298, ED269-270, L328 or S267 of a human IgG1, IgG2, IgG3 or IgG4 heavy chain polypeptide (Eu numbering). In an embodiment, the substitution in the CH2 domain is selected from: QYN295-297 substituted with NST, YNS296-298 substituted with NSS, ED269-270 substituted with AA, L328 substituted with F and S267 substituted with E (Eu numbering). In an embodiment, the substitution in the CH2 domain is QYN295-297 substituted with NST. In an embodiment, the substitution in the CH2 domain is YNS296-298 substituted with NSS. In an embodiment, the substitution in the CH2 domain is ED269-270 substituted with AA. In an embodiment, the substitution in the CH2 domain is L328 substituted with F and S267 substituted with E. In an embodiment, the substitution in the CH2 domain is L328 substituted with F. In an embodiment, the substitution in the CH2 domain is S267 substituted with E. In an embodiment, the polypeptide is an IgG2, IgG3 or IgG4 wherein modification is in the CH2 domain and wherein the modification is a substitution at a position corresponding to Q274 of a human IgG2, IgG3 or IgG4 heavy chain polypeptide (Eu numbering). In an embodiment, the substitution in the CH2 domain is Q274 substituted with K. In an embodiment, the polypeptide is an IgG1, wherein the modification is in the CH2 domain and wherein the modification is a substitution at a position corresponding to K274 of a human IgG1 heavy chain polypeptide (Eu numbering). In an embodiment, the substitution in the CH2 domain is K274 substituted with Q. In an embodiment, the polypeptide is an IgG1 or IgG3, and wherein the modification is in the lower hinge and wherein the modification is a substitution at a position corresponding to LL234-235. In an embodiment, the substitution in the lower hinge is LL234-235 substituted with AA. In an embodiment, the polypeptide is an IgG4 and wherein the modification is in the lower hinge and wherein the modification is a substitution at a position corresponding to FL234-235. In an embodiment, the substitution in the lower hinge is FL234-235 substituted with AA. In an embodiment, the polypeptide is an IgG2, and wherein the modification is in the CH2 domain and wherein the modification is a substitution at a position corresponding to PVA234-236. In an embodiment, the substitution in the lower hinge is selected from: PVA234-235 substituted with AAG.

In an embodiment, the polypeptide is an IgG1, IgG2, IgG3 or IgG4 and wherein the modification is in the CH3 domain and wherein the modification is a substitution at a position corresponding to E430 or H435 of a human IgG1, IgG2, IgG3 or IgG4 heavy chain polypeptide (Eu numbering). In an embodiment, the substitution in the CH3 domain is selected from: E430 substituted with G, and H435 substituted with A.

In an embodiment, the polypeptide is an IgG1, IgG2, IgG3 or IgG4 and wherein the modification is the CH2 and the CH3 domain. In an embodiment, the polypeptide is an IgG1 and wherein the modification is the CH2 and the CH3 domain. In an embodiment, the polypeptide is an IgG2 and wherein the modification is the CH2 and the CH3 domain. In an embodiment, the mutation in the CH2 domain is PVA233-236 substituted with EFLG and the modification in the CH3 domain is E430G. In an embodiment, the mutation in the CH2 domain is PVA233-236 substituted with ELLG and the modification in the CH3 domain is E430G. In an embodiment, the mutation in the CH2 domain is PVA233-236 substituted with EFEG and the modification in the CH3 domain is E430G. In an embodiment, the polypeptide is an IgG2, mutation in the CH2 domain is PVA233-236 substituted with EFLG and the modification in the CH3 domain is E430G. In an embodiment, the polypeptide is an IgG2, the mutation in the CH2 domain is PVA233-236 substituted with ELLG and the modification in the CH3 domain is E430G. In an embodiment, the polypeptide is an IgG2, the mutation in the CH2 domain is PVA233-236 substituted with EFEG and the modification in the CH3 domain is E430G.

In an embodiment, the polypeptide is an IgG3, the modification is in the CH3 domain and wherein the modification is a substitution at a position corresponding to N392, M397 and R435 of a human IgG3 heavy chain polypeptide (Eu numbering). In an embodiment, the substitution in the CH3 domain comprises N392 substituted with K, M397 substituted with V and R435 substituted with H (Eu numbering).

In an embodiment, the polypeptide is an IgG2, the C1q binding modification is in the CH1 domain and wherein the modification is a substitution at a position corresponding to C131 of the amino acid sequence of a human IgG1 heavy chain polypeptide substituted with a S (Eu numbering).

In an embodiment, the polypeptide is an IgG2 and the C1q binding modification is PVA233-236 substituted with EFLG, C131 substituted with S, and C220 substituted with S.

In an embodiment, the polypeptide is an IgG2 and the C1q binding modification is PVA233-236 substituted with EFLG, C131 substituted with S, and C219 substituted with S.

In an embodiment, the polypeptide is an IgG2 and the C1q binding modification is PVA233-236 substituted with EFLG, C131 substituted with S, and ERKCCVE substituted with IgG1 216-225EPKSCDKTHT.

In an embodiment, wherein the polypeptide is an IgG1 and the C1q binding modification is PVA233-236 substituted with EAAGG.

In an embodiment, the polypeptide is an IgG1 and the C1q binding modification is K274 substituted with a Q.

In an embodiment, the polypeptide is an IgG2, IgG3 or IgG4 and the C1q binding modification is Q274 substituted with K.

In some embodiments, the C1q binding modification is not E430 substituted with G. In some embodiments, the C1q binding modification is not L328 substituted with F or S267 substituted with E. In some embodiments, the C1q binding modification is not L328 substituted with F and S267 substituted with E.

In an embodiment, the immunotherapeutic protein is an anti-CD20 antibody. In an embodiment, the immunotherapeutic protein is an anti-trinitrophenyl antibody (anti-TNP antibody). In an embodiment, the immunotherapeutic protein is selected from: rituximab, ofatumumab, obinutuzumab, isatuximab, trastuzumab and pertuzumab. In an embodiment, the immunotherapeutic protein is selected from: rituximab, trastuzumab and pertuzumab. In an embodiment, the immunotherapeutic protein is rituximab.

In an embodiment, the immunotherapeutic protein is trastuzumab. In an embodiment, the immunotherapeutic protein is pertuzumab. In an embodiment, the immunotherapeutic protein is selected from: rituximab (Rit), Rit-IgG2 FLGG HF, Rit-IgG2-FLGG EG, Rit-IgG2-LLGG HF, Isa-IgG2-M1-H429F, Isa-IgG2-M2-H429F, Isa-IgG2-M3-H429F, S2P6-IgG2-M1-H429F, S2P6-IgG2-M2-H429F and S2P6-IgG2-M3-H429F. In an embodiment, the immunotherapeutic protein comprises Rit-IgG2-FLGG HF. In an embodiment, the immunotherapeutic protein comprises Rit-IgG2 FLGG EG. In an embodiment, the immunotherapeutic protein comprises Rit-IgG2-LLGG HF. In an embodiment, the immunotherapeutic protein comprises Isa-IgG2-M1-H429F. In an embodiment, the immunotherapeutic protein comprises Isa-IgG2-M2-H429F. In an embodiment, the immunotherapeutic protein comprises Isa-IgG2-M3-H429F. In an embodiment, the immunotherapeutic protein comprises S2P6-IgG2-M1-H429F.

In an embodiment, the immunotherapeutic protein comprises S2P6-IgG2-M2-H429F. In an embodiment, the immunotherapeutic protein comprises S2P6-IgG2-M3-H429F.

In an embodiment, the immunotherapeutic protein comprises: a) H429 substituted with an aromatic amino acid or cyclic amino acid wherein the C1q modification is PVA233-236 substituted with EFLG; b) H429 substituted with an aromatic amino acid or cyclic amino acid and wherein the C1q modification is PVA233-236 substituted with ELLG; c) H429 substituted with a hydrophobic amino acid and wherein the C1q modification is PVA233-236 substituted with EFLG; and d) H429 substituted with a hydrophobic amino acid and wherein the C1q modification is PVA233-236 substituted with ELLG.

In an embodiment, the immunotherapeutic protein is an IgG2 and wherein the IgG2 comprises a further mutation producing a disulfide between the light chain and upper hinge region of the polypeptide. In an embodiment, the disulfide bond is formed between C214 and C219. In an embodiment, the disulfide bond is formed between C214 and C220. In an embodiment, the further mutation is M1, substitution of C131 with serine and substitution of C220 with serine. In an embodiment, the further mutation is M2, substation of C131 with serine and substitution of C219 with serine. In an embodiment, the further mutation is M3, substation of C131 with serine and substitution of ERKCCVE in the IgG2 upper hinge with EPKSCDKTHT.

In an embodiment, the immunotherapeutic protein comprises modifications selected from: a) H429 substituted with F and wherein the C1q modification comprises PVA233-236 substituted with EFLG; b) H429 substituted with F and wherein the C1q modification comprises PVA233-236 substituted with ELLG; c) H429 substituted with Q and wherein the C1q modification comprises PVA233-236 substituted with EFLG; d) H429 substituted with Q and wherein the C1q modification comprises PVA233-236 substituted with ELLG; e) H429 substituted with E and wherein the C1q modification comprises PVA233-236 substituted with EFLG; f) H429 substituted with E and wherein the C1q modification comprises PVA233-236 substituted with ELLG; g) H429 substituted with S and wherein the C1q modification comprises PVA233-236 substituted with EFLG; h) H429 substituted with S and wherein the C1q modification comprises PVA233-236 substituted with ELLG; i) H429 substituted with A and wherein the C1q modification comprises PVA233-236 substituted with EFLG; j) H429 substituted with A and wherein the C1q modification comprises PVA233-236 substituted with ELLG; k) H429 substituted with Y and wherein the C1q modification comprises PVA233-236 substituted with EFLG; 1) H429 substituted with Y and wherein the C1q modification comprises PVA233-236 substituted with ELLG; m) H429 substituted with F, wherein the C1q modification is PVA233-236 substituted with EFLG, C131 substituted with serine, and C220 substituted with serine; n) H429 substituted with F, wherein the C1q modification is PVA233-236 substituted with EFLG, C131 substituted with serine, and C219 substituted with serine; o) H429 substituted with F, wherein the C1q modification is PVA233-236 substituted with EFLG, C131 substituted with serine, and ERKCCVE substituted with IgG1 EPKSCDKTHT; p) H429 substituted with F, wherein the C1q modification is PVA233-236 substituted with ELLG, C131 substituted with serine, and C220 substituted with serine; q) H429 substituted with F, wherein the C1q modification is PVA233-236 substituted with ELLG, C131 substituted with serine, and C219 substituted with serine; and r) H429 substituted with F, wherein the C1q modification is PVA233-236 substituted with ELLG, C131 substituted with serine, and ERKCCVE in the IgG2 upper hinge substituted with EPKSCDKTHT.

In an embodiment, the immunotherapeutic protein comprises H429 substituted with F and wherein the C1q modification comprises PVA233-236 substituted with EFLG. In an embodiment, the immunotherapeutic protein comprises H429 substituted with F and wherein the C1q modification comprises PVA233-236 substituted with ELLG. In an embodiment, the immunotherapeutic protein comprises H429 substituted with Q and wherein the C1q modification comprises PVA233-236 substituted with EFLG. In an embodiment, the immunotherapeutic protein comprises H429 substituted with Q and wherein the C1q modification comprises PVA233-236 substituted with ELLG. In an embodiment, the immunotherapeutic protein comprises H429 substituted with E and wherein the C1q modification comprises PVA233-236 substituted with EFLG. In an embodiment, the immunotherapeutic protein comprises H429 substituted with E and wherein the C1q modification comprises PVA233-236 substituted with ELLG. In an embodiment, the immunotherapeutic protein comprises H429 substituted with S and wherein the C1q modification comprises PVA233-236 substituted with EFLG. In an embodiment, the immunotherapeutic protein comprises H429 substituted with S and wherein the C1q modification comprises PVA233-236 substituted with ELLG. In an embodiment, the immunotherapeutic protein comprises H429 substituted with A and wherein the C1q modification comprises PVA233-236 substituted with EFLG. In an embodiment, the immunotherapeutic protein comprises H429 substituted with A and wherein the C1q modification comprises PVA233-236 substituted with ELLG. In an embodiment, the immunotherapeutic protein comprises H429 substituted with Y and wherein the C1q modification comprises PVA233-236 substituted with EFLG. In an embodiment, the immunotherapeutic protein comprises H429 substituted with Y and wherein the C1q modification comprise PVA233-236 substituted with ELLG. In an embodiment, the immunotherapeutic protein comprises H429 substituted with F, wherein the C1q modification is PVA233-236 substituted with EFLG, C131 is substituted with serine, and C220 is substituted with serine. In an embodiment, the immunotherapeutic protein comprises H429 substituted with F, wherein the C1q modification is PVA233-236 substituted with EFLG, C131 is substituted with serine, and C219 is substituted with serine. In an embodiment, the immunotherapeutic protein comprises H429 substituted with F, wherein the C1q modification is PVA233-236 substituted with EFLG, C131 is substituted with serine, and ERKCCVE in the IgG2 upper hinge is substituted with IgG1 216 225EPKSCDKTHT. In an embodiment, the immunotherapeutic protein comprises H429 substituted with F, wherein the C1q modification is PVA233-236 substituted with ELLG, C131 is substituted with serine, and C220 is substituted with serine. In an embodiment, the immunotherapeutic protein comprises H429 substituted with F, wherein the C1q modification is PVA233-236 substituted with ELLG, C131 is substituted with serine, and C219 is substitute with serine. In an embodiment, the immunotherapeutic protein comprises H429 substituted with F, wherein the C1q modification is PVA233-236 substituted with ELLG, C131 is substituted with serine, and ERKCCVE in the IgG2 upper hinge is substituted with IgG1 216-225EPKSCDKTHT.

In an embodiment, the C1q binding modification increases complement-based lysis compared to a control immunotherapeutic protein lacking the C1q binding modification. In an embodiment, the C1q binding modification decreases complement-based lysis compared to a control immunotherapeutic protein lacking the C1q binding modification. In an embodiment, the control antibody is a wild type IgG2. In an embodiment, the control antibody is a wild type IgG1.

In an embodiment, the immunotherapeutic protein comprises at least two C1q binding modifications. In an embodiment, the immunotherapeutic protein comprises at least three C1q binding modifications.

In some embodiments, the immunotherapeutic protein as described herein may comprise one or more further mutation/s (e.g. amino acid substitution).For example, the Fc component may comprise one or more sequence mutation known to those skilled in the art (see, for example, the examples listed in Table 1 of Wang et al., Protein Cell 9(1):63-73, 2018; the entire disclosure of which is herein incorporated by reference) such as, for example: mutations that may modulate FcγR binding (e.g. S239D/I332E of IgG1 (Eu numbering) which increase FcγRIIIa binding); mutations to improve antibody-dependent cellular cytotoxicity (ADCC) such as S239D/I332E (Lazar G A et al., Proc Natl Acad Sci USA 103(11):4005-4010, 2006), opsonic phagocytosis (e.g. G236A/S239D/I332E; Richards J O et al., Mol Cancer Ther 7:2517-2527, 2008) or complement activation (e.g. H268F/S324T; Moore G L et al., MAbs 2:181-189, 2010), or decrease effector function (e.g. IgG1: L234A/L235A; IgG4: F234A/L235A; Xu D et al., Cell Immunol 200(1):16-26, 2000); and mutations which confer enhanced binding to the neonatal Fc receptor (FcRn) such as M252Y/S254T/T256E (Dall'Acqua W F et al., J Immunol 169(9):5171-5180, 2002) to increase in vivo half-life and thereby improve pharmacokinetics (PK).

Based on the observations of the C1q binding by the mAbs as shown in FIG. 57, FIG. 58 and FIG. 59, FIG. 71, FIG. 78, FIG. 85 and grouping in Table 6 and the sequence alignment of FIG. 56 and Tables 7, 8, 9 additional mutations could be incorporated onto an IgG2 backbone particularly mutation of the cysteine residues involved in disulphide bonding of light chain to heavy chain and or between heavy chains (at position 131 in CH1 or in the upper hinge at position 219 and 220 of IgG1, IgG2, IgG4 or IgG4 Eu numbering). Such mutations could be used alone or in combination for example with a C1q binding modification e.g. C131S in CH1 of IgG2 or S131C in CH1 of IgG1, IgG3 or IgG4. In some embodiments, the C1q binding modification is used in combination with C219S and/or C220S. In some embodiments, the C1q binding modification is used in combination with C219S. In some embodiments, the C1q binding modification is used in combination with C220S. In some embodiments, the C1q binding modification is used in combination with C219S and/or C220S in an IgG2. Other point mutations might also be useful. For example at position 274 of IgG1 the mutation K274Q may result in recreased C1q binding (FIG. 85) and is also inferred from the reduced CDC activity of this mutant compared with its IgG1-WT counterpart (FIG. 84). This point mutation may be used in combination with other mutations such as those targeting the disulphide bonds in CH1 or the upper hinge.

In an embodiment, the further mutation is a substitution selected from K274 with Q, Q274 with K, C219 with S, 5219 with C, and C220 with S.

In an embodiment, the polypeptide is an IgG2 and wherein the polypeptide comprises a further mutation that prevents formation of an intra-heavy chain disulfide bond in the upper hinge region of the polypeptide. In an embodiment, the intra-heavy chain disulfide bond is between C131 and C219, or between C131 and C220. In an embodiment, the polypeptide comprises a mutation at one or more of positions C131, C219 or C220 and wherein the mutation is a substitution to an amino acid other than cysteine. In one example, the substitution is to serine. In one example, the mutation is at positions C131 and C219 or C131 and C220.

In an embodiment, the polypeptide is an IgG2 and wherein the polypeptide comprises a further mutation that forms a disulfide bond between the light chain and upper hinge region of the polypeptide. In an embodiment, the disulfide bond is formed between C214 of the light chain and C219 of the heavy chain or between C214 of the light chain and C220 of the heavy chain. In an embodiment, the further mutation is M1, substitution of C131 with S and a substitution of C220 with S. In an embodiment, the further mutation is M2, substitution of C131 with S and substitution of C219 with S. In an embodiment, the further mutation is M3, a substation of C131 with S and substitution of ERKCCVE in the IgG2 upper hinge with EPKSCDKTHT.

In an embodiment, the immunotherapeutic protein is an anti-CD20 antibody. In an embodiment, the immunotherapeutic protein is an anti-trinitrophenyl antibody (anti-TNP antibody).

Other mutations that may be included in the immunotherapeutic protein component include mutations to enhance complement activation such as an amino acid mutation at a position(s) corresponding to K447 of the amino acid sequence of the human IgG1 heavy chain polypeptide (Eu numbering); in particular, K447X, where X is selected from null (i.e. K447del; an amino acid deletion or truncation of the Fc component), and glutamate (i.e. K447E) (see van der Bremer ETJ et al., mAbs 7(4):672-680, 2015). Further, the immunotherapeutic protein component may also include mutations which modulate glycosylation (e.g. a mutation at a position corresponding to Asn297 of the amino acid sequence of the human IgG1 heavy chain polypeptide (Eu numbering) such as N297A, N297Q or N297G (Wang et al., 2018 supra) to provide a site with modified glycosylation (e.g. the lack of glycan at position 297) to abolish FcγR and complement C1 binding and/or activation.

Alternatively, the immunotherapeutic protein component may also be treated to achieve modified glycosylation by producing the immunotherapeutic protein in the presence of kifunensine (a mannosidase inhibitor which prevents normal maturation of the N-linked glycan including core fucosylation of the N-linked glycan); a modification that specifically enhances activity via FcγRIIIa.

Further, an Fc component lacking core fucosylation of the Asn297 glycan can also be achieved by culturing host cells expressing the immunotherapeutic protein with inhibitors of fucosylation (e.g. 2-fluoro peracetylated fucose, or similar) or by the expression of enzymes that modify glycosylation pathways (e.g. GDP-6-deoxy-D-lyxo-4-hexulose reductase; Neha M et al., J Biotech 5:100015, 2020) or by modification of these pathways by gene suppression (e.g. siRNA silencing of the α−1,6-Fucosyltransferase fucosyl transferase gene FUT8; Imai-Nishiya H et al., BMC Biotechnol 7:84, 2007) or knock-out (Yamane-Ohnuki N et al., Biotechnol Bioeng 87:614-622, 2004.)

The immunotherapeutic protein as described herein may be monomeric, dimeric or oligomeric in solution (e.g. in physiological saline at neutral pH such as physiological pH of about 7.4).

For example, in some embodiments, the immunotherapeutic protein comprises a monomer in physiological saline, wherein each monomer comprises one copy of the Fc region component, while in other embodiments, the immunotherapeutic protein comprises a dimer in physiological saline wherein the Fc region components self-associate (i.e. to form the dimer) either by non-covalent bonding such as hydrogen bonding or through the formation of disulphide bonds through one or more cysteine (C) residue, particularly where situated within the hinge sequence, especially the core hinge sequence (if present) (Yoo E M et al., J Immunol 170:3134-3138, 2003). Thus, in some embodiments, the Fc region component comprises a core hinge sequence to enable the immunotherapeutic protein to self-associate (i.e. to form a dimer) through the formation of inter-chain disulphide bonds between one or more cysteine (C) residues of the core hinge sequence of two Fc region components, while in some other embodiments, the Fc region component comprises a CH3 domain to enable CH3:CH3 self-association by non-covalent interactions, or Fc region component comprises a CH3 domain and CH2 domain to enable self-association by CH2:CH2 and CH3:CH3 non-covalent interactions. It is to be noted that in the Examples and Figures hereinafter, dimeric forms of the immunotherapeutic protein are considered to be single molecules (i.e. each comprising two copies of the protein dimerised through the Fc region components) and are referred to as monomers/monomeric.

In other embodiments, the immunotherapeutic protein comprises an oligomer in physiological saline (at neutral pH such as physiological pH of about 7.4), wherein the immunotherapeutic protein comprises an amino acid substitution at the position corresponding to H429 of the amino acid sequence of the human IgG1 heavy chain polypeptide, which enables self-association of the Fc or Fc-like fragment comprising the mutated Fc components into soluble oligomeric forms in physiological saline (e.g. oligomeric forms comprising, for example, 3 copies, 4 copies, 5 copies, 6 copies or 12 copies assembled from, for example, dimeric forms of the immunotherapeutic protein, such that, in some particular embodiments, the oligomeric form of the immunotherapeutic protein may comprise six dimeric proteins (i.e. a hexameric form) comprising, in total, 12 copies of the immunotherapeutic protein). In such embodiments, the amino acid substitution at the position corresponding to H429 of the amino acid sequence of the human IgG1 heavy chain polypeptide may be:
H429X¹, where X¹ is selected from tyrosine (i.e. H429Y), methionine, isoleucine, leucine, tryptophan and valine;
   but preferably, the immunotherapeutic protein comprises an H→Y (i.e. H429Y) substitution.

In yet other embodiments, the immunotherapeutic protein may form an oligomer upon binding to a relevant target through “on target” oligomerisation. Such oligomerisation may occur with an immunotherapeutic protein that is monomeric or dimeric in physiological saline (at neutral pH such as physiological pH of about 7.4), and comprises an amino acid substitution at the position corresponding to H429 of the amino acid sequence of the human IgG1 heavy chain polypeptide which enables on target oligomerisation into oligomeric forms (e.g. forms comprising 3 copies, 4 copies, 5 copies, 6 copies or 12 copies (e.g. a hexamer of dimeric forms) of the immunotherapeutic protein). In such embodiments, the amino acid substitution at the position corresponding to H429 of the amino acid sequence of the human IgG1 heavy chain polypeptide may be:
H429X², where X² is selected from phenylalanine (i.e. H429F), glutamate, glutamine and serine;
   but preferably, the immunotherapeutic protein comprises an H→F (i.e. H429F) substitution.

Alternatively, oligomeric forms of the immunotherapeutic protein as described herein may be produced by employing other techniques well known to those skilled in the art such as, for example, the use of Fc multimeric forms (Stradomers™) comprising linked multimerisation domain (MD) sequences from the hinge region of human IgG2 or the isoleucine zipper (ILZ) to the N- or C-terminus of murine IgG2a (Fitzpatrick E A et al., Front Immunol 11, article 496, 2020), and the use of multimerisation sequences from IgM (Melcheil et al., Sci Rep 1:124 doi:10.1038/srep0012, 2011), or docking and dimerisation sequences from unrelated proteins such as cyclic adenosine monophosphate-dependent protein kinase and A-kinase anchoring proteins (Rossi E A et al., Bioconjug Chem 23(3):309-323, 2012).

It has further been found that the immunotherapeutic protein as described herein may show enhanced binding of the neonatal Fc receptor (FcRn) by virtue of an amino acid substitution at a position corresponding to H429 of the amino acid sequence of human IgG1 heavy chain (Eu numbering). It is anticipated that this will mean that the immunotherapeutic protein will show a greater in vivo half-life (i.e. relative to an equivalent immunotherapeutic protein with no mutation at the H429 position) and thereby improved pharmacokinetics (PK), since FcRn is known to “recycle” antibodies so as to control how long they last in the body (Ward ES and R J Orber, Trends Pharmacol Sci 39(10):892-904, 2018).

Dimeric Immunotherapeutic Proteins (e.g. Immunoglobulin Molecules)

The immunotherapeutic protein as described herein may be a dimeric immunotherapeutic protein comprising an immunoglobulin molecule, wherein the immunoglobulin molecule comprises first and second immunoglobulin heavy chain polypeptides which each comprise an Fc region component comprising at least a CH3 domain (or at least a CH4 domain). The Fc region components of the first and second immunoglobulin heavy chain polypeptides may self-associate (i.e. to form the dimer) either by non-covalent bonding such as hydrogen bonding or through the formation of disulphide bonds through one or more cysteine (C) residue, particularly where situated within the hinge sequence, especially the core hinge sequence (if present) (Yoo E M et al., J Immunol 170:3134-3138, 2003), or otherwise be linked by, for example, a cross-linker compound such as those mentioned above (e.g. by cross-linking the CH3 domains of the Fc region components). As such, the immunotherapeutic protein may be an immunoglobulin molecule such as, for example, an antibody or antibody derivative such as an scFv-Fc (wherein a dimer of scFv polypeptides is formed through dimerisation of Fc region components), mini-body (wherein a dimer of scFv polypeptides is formed through linking CH3 domains/CH4 domains), or any of the other suitable Fc-containing antibodies or derivatives known to those skilled in the art (e.g. as summarised in the “Periodic Table of Antibodies” mentioned above).

Accordingly, the dimeric immunotherapeutic protein may further comprise at least one antigen recognition structure or, in other words, antigen binding region (e.g. an scFv or an antigen binding region comprising variable domains V_{L} and V_{H} such as a Fab fragment). The antigen binding region(s) may specifically bind to an antigen or epitope of therapeutic significance; for example, a cancer-associated antigen such as a cancer antigen present on the surface of a cancerous cell (e.g. a cell surface antigen differentially expressed and/or present in cancer cells such as the CD20, CD38 and CD52 antigens found on the surface of CLL cells, and mucins (e.g. MUC-1) or carbohydrate (e.g. Lewis X) overexpressed in some breast and pancreatic cancers), an autoantigen (e.g. an autoantigen associated with SLE or multiple sclerosis (MS)), an allergen (e.g. bee venom), an antigen associated with other inflammatory diseases such as immune complex vasculitis, an antigen from a transplanted tissue or organ or an antigen of an infectious agent such as an antigen of a bacterial, yeast, parasite or viral pathogen (e.g. an antigen of the SARS-CoV-2 virus, Middle East respiratory syndrome coronavirus (MERS-CoV), respiratory syncytial virus (RSV) or dengue virus). In other examples, the antigen binding region(s) may specifically bind to a cell surface molecule known to induce cell proliferation (i.e. the cell surface molecule may be, for example, CD3 or CD28) and/or stimulate an inhibitory pathway to inhibit or reduce cell responses (i.e. the cell surface molecule may be, for example, an immune checkpoint molecule such as 4-1BB (CD137), cluster of differentiation (CD40, CD154), OX40 receptor (TNFRSF4, CD134), tumour necrosis factor receptor type II (TNFR2, CD120b), glucocorticoid-induced TNFR-related protein (GITR, TNFRSF18, CD357), cluster of differentiation 27 (CD27), T cell immunoglobulin and mucin-domain containing-3 (TIM-3), B and T lymphocyte attenuator (BTLA, CD272), lymphocyte activation gene-3 (LAG3, CD223), cytotoxic T-lymphocyte-associated protein 4 (CTLA4, CD152), inducible T-cell costimulatory (ICOS, CD278), cluster of differentiation 28 (CD28), T cell immunoreceptor with Ig and ITIM domains (IGIT, Vstm3), programmed death-ligand 1 (PDL-1, CD274) and programmed cell death protein 1 (PD-1, CD279); and wherein oligomerisation of the immunotherapeutic protein may bring about enhanced cross-linking (e.g. super-clustering) and, in turn, enhanced signalling to induce, for example, enhanced cell proliferation or inhibited/reduced cell responses (i.e. through enhanced stimulation of an inhibitory pathway). In yet other examples, the antigen binding region(s) may specifically bind to a cell surface molecule known to induce a cellular response selected from stimulation of an inhibitory pathway to inhibit or reduce responses. In still yet other examples, the antigen binding region(s) may specifically bind to a cell surface molecule which induces cell death when engaged by a ligand or other molecules such as agonistic mAbs. Such cell surface molecules include, for example, those of the TNF-receptor superfamily (also known as TNFRSF) including tumour necrosis factor receptor 1 (TNFR1, TNFRSF1A, CD120a), Fas (CD95, APO-1), Death Receptor 3 (DR3, TNFRSF25), Death Receptor 4 (DR4, CD261, TNFRSF10A, TRAILR1), Death Receptor 5 (DR5, CD262, TNFRSF10B, TRAILR2) and Death Receptor 6 (CD358, TNFRSF21). For example, under physiological conditions, the binding of TNF-related apoptosis-inducing ligand (TRAIL) to its receptor, DR5 (Carneiro B A et al., Nat Rev Clin Oncol 17(7):395-417, 2020) or the binding of the FAS ligand to its receptor, FAS (CD95) on the cell surface induces oligomerisation (Leukocyte and Stromal Cell molecules: The CD Markers, by Zola H et al., page 195, John Wiley & Sons, 2007) that initiates signalling leading to cell death. Other molecules (not related to the TNFRSF) which can induce apoptotic signals leading to cell death when cross-linked by, for example, mAbs or fragments thereof include CD38 (see Gambles M T et al., Molecules 26(15):4658, 2021), CD20 (see Shan D et al., Cancer Immunol Immunother 48:673-683, 2000; and Cardarelli P M et al., Cancer Immunol Immunother 51:15-24, 2002) and CD52 (see Rowan W et al., Immunology 95:427-436, 1998). These molecules can also be advantageously targeted by a dimeric immunotherapeutic protein such that oligomerisation may bring about enhanced cross-linking and induction of apoptotic signals to bring about cell death as may be desired in a target cell.

In some embodiments, an immunoglobulin molecule according to the present disclosure may comprise two antigen binding regions, each of which specifically binds to different antigens or epitopes. As used herein, an “antigen” is any substance that causes the body to make an immune response (antibodies) against that substance. As used herein, an “epitope” also known as an “antigenic determinant” is the part of an antigen that is recognized by an antigen recognition structure.

In some embodiments, an immunoglobulin molecule according to the present disclosure that is an antibody, may be of, for example, an IgD, IgE or IgM isotype, but preferably, will be of an IgA or IgG isotype, such as an antibody of any of the human IgAQ1, IgA2, IgG1, IgG2, IgG3 and IgG4 sub-types.

The first and second immunoglobulin heavy chain polypeptides of a dimeric immunotherapeutic protein comprising an immunoglobulin molecule may be the same (i.e. a homodimer) or different (i.e. a heterodimer).

In some embodiments, an immunoglobulin molecule according to the present disclosure that is an antibody, may be an antibody that forms oligomers either through oligomerisation into oligomers (e.g. hexamers) through self-association of antibodies either in solution (e.g. in physiological saline at neutral pH such as physiological pH of about 7.4) or upon binding to a relevant target (e.g. an antigen to which an antibody mutant is directed) through “on target” oligomerisation.

For example, and while not wishing to be bound by theory, it is considered that an immunoglobulin molecule that is an antibody which includes H429Y amino acid substitution in the Fc region component, may form oligomers in solution (e.g. in physiological saline at physiological pH) which can lead to an overall increase in the strength of binding (for example, enhanced avidity) to a target binding partner (e.g. a cancer antigen, antigen of a bacterial or viral pathogen, or other soluble target molecule or molecular complex). Thus, in the context of an antibody directed against a cancer antigen present on the surface of a cancerous cell, an antibody according to the present disclosure (in the form of an oligomer in solution) may show enhanced ability to bind to the cancerous cells leading to their destruction by, for example, CDC by complement or by phagocytosis by macrophages and/or ADCC; and in the context of an antibody directed against a soluble target molecule or molecular complex, an antibody according to the present disclosure (in the form of an oligomer in solution) may show enhanced ability to bind to the soluble target molecule or molecular complex leading to their removal by phagocytosis by macrophages.

On the other hand, it is considered that an immunoglobulin molecule that is an antibody which includes an H429F amino acid substitution in the Fc region component, may form oligomers upon binding to a relevant target such that the Fc components assembled “on target” present a stabilised optimal platform for binding with, for example, enhanced avidity to the C1q complement protein complex, and thereby lead to complement activation and, in turn, complement-dependent cytotoxicity (CDC). Thus, in the context of an antibody directed against a cancer antigen present on the surface of a cancerous cell, an antibody according to the present disclosure may show enhanced capability to activate complement on the cancerous cells leading to their destruction.

In some embodiments, an immunoglobulin molecule according to the present disclosure may be provided or used as a first immunoglobulin molecule with a first antigen binding region directed to a first antigen, in combination with a second immunoglobulin molecule (according to the present disclosure) with a second antigen binding region directed to a second antigen. By way of example only, in such a combination, the first antigen may be cancer antigen present on the surface of a cancerous cell (e.g. CD38 found on the surface of CLL cells) and the second antigen may be a death receptor (e.g. DR5).

Oligomerisation of the first and second immunoglobulin molecules may lead to the formation of hetero-oligomers such as hetero-hexamers wherein the different cell surface targets are incorporated into the cluster which may lead to, for example, the enhanced induction of apoptotic signals to bring about cell death (e.g. where a death receptor has been targeted) with target cell specificity achieved via binding of, for example, a cancer antigen present on a cancerous cell.

### Fusion/Conjugate Immunotherapeutic Proteins

The immunotherapeutic protein as described herein may comprise a fusion protein or protein conjugate comprised of a partner polypeptide linked to an Fc region component comprising at least a CH3 domain (or at least a CH4 domain).

A fusion protein or protein conjugate according to the present disclosure may be monomeric, dimeric or oligomeric. For example, in some embodiments, the fusion protein or protein conjugate comprises a monomer, wherein each monomer comprises one copy of the partner polypeptide (or a fragment thereof) and one copy of the Fc region component, while in other embodiments, the immunotherapeutic protein comprises a dimer wherein the Fc region components self-associate (i.e. to form the dimer) either by non-covalent bonding such as hydrogen bonding or through the formation of disulphide bonds through one or more cysteine (C) residue, particularly where situated within the hinge sequence, especially the core hinge sequence (if present) (Yoo E M et al., J Immunol 170:3134-3138, 2003). In such a dimer, the immunotherapeutic protein comprises two Fc region components (associated to one another to form, for example, an Fc fragment or Fc-like fragment) and two fused/conjugated partner polypeptides (or two fragments thereof), and as such may be considered as being bivalent in respect of the partner polypeptide (or fragment thereof). The two fused/conjugated partner polypeptides (or two fragments thereof) of a dimeric fusion protein or protein conjugate may be the same (i.e. a homodimer) or different (i.e. a heterodimer). In some embodiments of such a dimer, as depicted in FIG. 53A, the fusion protein is antibody-like (Ab-like) and may have, for example a H₂ format or H₂L₂ format (where the monomeric fusion proteins are produced with a light (L) chain, which in turn may be optionally fused/conjugated with a partner polypeptide (such as a target recognition structure)). As can be readily appreciated, each target recognition structure of an Ab-like molecule with a H₂ or H₂L₂ format may be the same or different such as “X1 X1 X1 X1”, “X1 X1 X1 X2”, “X1 X1 X2 X2”, “X1 X1 X2 X3” or “X1 X2 X3 X4”, where X1, X2, X3 and X4 each represent a different target recognition structure. Also, one or more of the target recognition structures may be replaced with an alternative partner polypeptide type such as an enzyme(s) or reporter molecule(s).

The partner polypeptide may provide the immunotherapeutic protein with a beneficial function and/or characteristic.

In some embodiments, the partner polypeptide may be a cell surface receptor polypeptide (or a fragment thereof) or a co-receptor polypeptide (or fragment thereof).

For example, the partner polypeptide may be a cell surface molecule such as a cell surface receptor polypeptide (or a fragment thereof) that is capable of binding to a structural protein of a virus such that the immunotherapeutic protein may act as a “decoy” to block viral interaction and cellular entry of a virus to a host cell. Accordingly, some examples of such cell surface receptor polypeptides (or fragments thereof) that may comprise the partner polypeptide include the angiotensin-converting enzyme 2 (ACE2) ectodomain (ACE2 being the cellular entry receptor for SARS-CoV-2), nucleolin (the cellular entry receptor for RSV), dipeptidyl peptidase 4 (DPP4, CD26), Hsp70 (the cellular entry receptor for Japanese encephalitis virus), hepatitis A virus cellular receptor 1 (HAVCR1/TIM-1; the cellular entry receptor for Hepatitis A virus and Ebola virus), cluster of differentiation 155 (CD155; the cellular entry receptor for poliovirus), Glucose transporter 1 (GLUT1; the cellular entry receptor for human T cell leukaemia virus 1), Proto-oncogene tyrosine-protein kinase MER (MERTK; a host factor that promotes swine fever virus entry), TYRO3 Protein Tyrosine Kinase (TYRO3; a cell surface protein associated with lymphocytic choriomeningitis (LMCV) infection), AXL (a cell surface protein associated with lymphocytic choriomeningitis (LMCV) infection) and the cluster of differentiation 4 receptor (CD4 receptor; the cellular entry receptor for human immunodeficiency virus (HIV)), to name just a few. In embodiments where the partner polypeptide is a cell surface receptor polypeptide (or a fragment thereof) that is capable of binding to a structural protein of a virus, a point mutation at the position of the Fc region component corresponding to H429 of the amino acid sequence of human IgG1 heavy chain (Eu numbering) may, for example, provide the immunotherapeutic protein with an enhanced ability to provide an antiviral effect through CDC of infected cells (e.g. where the mutation is H429F) or confer upon the immunotherapeutic protein enhanced virus neutralisation (e.g. where the mutation is H429Y). In other examples, the partner polypeptide may be a ligand for a cell surface molecule known to induce cell proliferation (i.e. the cell surface molecule may be, for example, CD3 or CD28) and/or stimulate an inhibitory pathway to inhibit or reduce cell responses (i.e. the cell surface molecule may be, for example, an immune checkpoint molecule such as 4-1BB, CD40, OX40, TNFR2, GITR, CD27, TIM-3, BTLA, LAG3, CTLA4, ICOS, CD28, TIGIT, PDL-1 and PD-1; and wherein oligomerisation of the immunotherapeutic protein may bring about enhanced cross-linking (e.g. super cross-clustering) and, in turn, enhanced signalling to induce, for example, enhanced cell proliferation or inhibited/reduced cell responses (i.e. through enhanced stimulation of an inhibitory pathway). In yet other examples, the partner polypeptide may be a ligand for a cell surface molecule which induces cell death when engaged by a ligand or other molecules such as agonistic mAbs (e.g. the abovementioned cell surface molecules of the TNFRSF (e.g. TNFR1, Fas, DR3, DR4, DR5 and DR6) and other molecules such as CD38, CD20 (Shan et al., supra 2000; and Cardarelli et al., supra 2002) and CD52 (Rowan et al., supra 1998). Thus, in some embodiments, the immunotherapeutic protein as described herein may comprise a dimeric fusion protein or protein conjugate comprised of, for example, TRAIL (i.e. the ligand for DR4 or DR5) as the partner polypeptide linked to an Fc region component comprising at least a CH3 domain which includes an H429F amino acid substitution. Upon binding of the ligand to the cell surface molecule, oligomerisation of an immunotherapeutic protein of these embodiments may bring about enhanced cross-linking and induction of apoptotic signals to bring about cell death as may be desired in a target cell.

Where the partner polypeptide is a co-receptor polypeptide (or fragment thereof), the co-receptor polypeptide (or fragment thereof) may be, for example, C-X-C chemokine receptor type 4 (CXCR4), C-C chemokine receptor type 5 (CCR5) (co-receptors of the CD4 receptor which bind to the HIV viral glycoprotein gp120 and enable HIV to fuse with the host cell membrane), tetraspanin and occludin (which are co-receptors required to enable infection by Hepatitis C virus (HCV)), and Gas6 (which is a ligand of receptors (such as AXL and TYRO3) which together bind to phosphatidylserine displayed on viruses, including West Nile virus, Zika virus and Ebola virus among others, and facilitates host cell entry of such viruses), among many others.

In another example, the partner polypeptide may be a cell surface receptor polypeptide (or a fragment thereof) that is capable of binding to a ligand for the cell surface receptor. Accordingly, some examples of such polypeptides (or fragments thereof) include the cytotoxic T-lymphocyte-associated protein 4 (CTLA4) (or a soluble extracellular fragment thereof). CTLA4 functions as an immune checkpoint and downregulates immune responses. An immunotherapeutic protein comprising a fusion protein or protein conjugate comprised of a CTLA4 partner polypeptide (e.g. a CTLA4-Fc component fusion protein) may, by forming a dimer or oligomer, show enhanced binding to the CTLA4 ligand and thereby act as a decoy to produce various therapeutic effects for the potential treatment of tumours (e.g. melanoma and colorectal cancer) and various autoimmune diseases such as SLE and rheumatoid arthritis (RA). Further examples of suitable cell surface receptor polypeptides (or a fragment thereof such as an ectodomain) that are capable of binding to a ligand for the cell surface receptor, include other immune checkpoints (e.g. PD1) and other cytokine receptors such as interleukin-1 receptor (IL-1R), interleukin-6 receptor (IL-6R), tumour necrosis factor receptor-2 (TNFR2, also known as CD120b) or a receptor for a cytokine of the TGF-β superfamily (see the review in Czajkowsky D M et al., Mol Med 4(10):1015-1028, 2012). An immunotherapeutic protein comprising a fusion protein or protein conjugate comprised of IL-1R (or a fragment thereof) may potentially be used in an anti-IL-1 therapy for treatment of, for example, type 2 diabetes, and an immunotherapeutic protein comprising a fusion protein or protein conjugate comprised of IL-6R (or a fragment thereof) may potentially be used in an anti-IL-6 therapy for treatment of, for example, tumours and RA. An immunotherapeutic protein comprising a fusion protein or protein conjugate comprised of TNF-R2 or an ectodomain thereof may potentially be used in a treatment of RA or other inflammatory disease or condition.

While not wishing to be bound by theory, it is considered that a fusion protein or protein conjugate according to the present disclosure (whether in monomeric or dimeric form) which includes an H429Y amino acid substitution in the Fc region component may form oligomers in solution (e.g. in physiological saline at neutral pH such as physiological pH of about 7.4) that can lead to an overall increase in the strength of binding (for example, enhanced avidity) to a target binding partner (e.g. a structural protein of a virus or a ligand for a cell surface receptor, or other soluble target molecule or molecular complex). Thus, in the context of a fusion or conjugate protein which comprises a cell surface receptor polypeptide or a fragment thereof that is capable of binding to a structural protein of a virus, resulting in enhanced avidity of binding to the virus to provide enhanced virus neutralisation (possibly conferred by cross-linking and/or aggregating viral particles (virions)). However, it is considered that in at least some embodiments, such soluble oligomers are unable to substantially bind to Fc receptors and thus may be capable of virus neutralisation with little or no complement activation.

On the other hand, and again not wishing to be bound by theory, it is considered that a fusion protein or protein conjugate (whether in monomeric or dimeric form) which includes an H429F amino acid substitution in the Fc region component, may form oligomers upon binding to a relevant target (e.g. a structural protein of a virus or a ligand for a cell surface receptor) such that the Fc components assembled “on target” present a stabilised optimal arrangement for binding (with, for example, enhanced avidity) to the C1q complement protein complex, and thereby lead to complement activation and, in turn, enhanced complement-based effector functions such as complement-dependent cytotoxicity (CDC).

Immunotherapeutic proteins according to the present disclosure may be produced in accordance with any of the standard methodologies known to those skilled in the art. For instance, those skilled in the art can readily prepare an immunotherapeutic fusion protein by generating a construct, using standard molecular biology techniques, which comprises a polynucleotide sequence(s) encoding the fusion protein, introducing the construct into a suitable host cell (e.g. a human kidney (HEK) host cell or derivative thereof such as Expi293 cells (Thermo Fisher Scientific) for expression of the fusion protein or host animal (e.g. pig, monkey, rabbit or mouse), culturing the host cells according to standard culturing protocols and recovering the expressed fusion protein from the culture supernatant using, for example, any of the known suitable methodologies for purification (e.g. affinity chromatography (e.g. Protein A), ion exchange chromatography (IEX), size exclusion chromatography (SEC) and combinations thereof). In some embodiments, the immunotherapeutic protein is produced by a transgenic animal (the animal is modified to express the immunotherapeutic protein or nucleic acid as described herein). Similar methodologies can be used to prepare an immunotherapeutic protein which is an immunoglobulin molecule such as an antibody or an Ab-like molecule. That is, those skilled in the art can readily prepare a mutant antibody (i.e. an antibody including an amino acid substitution at a position corresponding to H429 of the amino acid sequence of human IgG1 heavy chain (Eu numbering) by generating a construct(s) which comprises a polynucleotide sequence(s) encoding the variable heavy (V_{H}) and light (V_{L}) region sequence of a suitable antibody (e.g. one including an antigen binding region that binds to an antigen of interest) and a constant heavy (CH) region from, for example, an IgG1 antibody, and incorporate into the CH3 region-encoding polynucleotide sequence by standard molecular biology techniques such as site-directed mutagenesis, polynucleotide sequence changes to encode a mutation at H429 (e.g. H429F and H429Y). As with the preparation of an immunotherapeutic fusion protein, the construct(s) can be introduced into a suitable host cell (e.g. a human kidney (HEK) host cell or derivative thereof), cultured according to standard culturing protocols and the expressed mutant antibody purified from the culture supernatant using, for example, any of the known suitable methodologies for purification such as affinity chromatography. Further, it has been found that where affinity chromatography (e.g. Protein A) is used in the purification, especially where the immunotherapeutic protein is in the form of an Ab-like molecule, the use of mild elution conditions such as the use of an elution buffer comprising a low concentration of arginine (e.g. less than 130 mM) and at less than or equal to pH 5, is advantageous so as to suppress the formation of aggregates. This can be achieved by, for example, standard liquid chromatography systems that can deliver a buffer gradient to the column, in this case, a linear gradient to, for example, 35% of 130 mM arginine (pH 4.0).

In some preferred embodiments, the recovery of an expressed immunotherapeutic protein according to the present disclosure is:
(i) preferably conducted under conditions of mildly acidic pH (e.g. a pH of less than neutral pH such as pH 6.5, preferably pH 5.5, and more preferably, pH 5.0) where it is desired that the immunotherapeutic protein be provided in a monomeric form; or
(ii) where it is desired that the immunotherapeutic protein be provided in a oligomeric form (e.g. as a hexamer), preferably conducted under conditions of substantially neutral pH (e.g. a pH in the range of 7.0 to 8.5, preferably 7.5 to 8.0, or more preferably, at physiological pH of about 7.4); or
(iii) preferably conducted using a method comprising affinity chromatography using an elution buffer comprising a low concentration of arginine (e.g. less than 130 mM) and at less than or equal to pH 5.0 (preferably about pH 4.0), especially where it is desired that the immunotherapeutic protein be provided as an antibody-like molecule.

In some particular embodiments, the recovery of an expressed immunotherapeutic protein according to the present disclosure comprises recovery by size exclusion chromatography (SEC), for example under conditions of mildly acidic pH for the production of monomeric forms of the immunotherapeutic protein, or under substantially neutral pH for the production of the immunotherapeutic protein in oligomeric forms. The SEC may, if desired, follow a recovery stage comprising ion exchange chromatography (IEX).

The US FDA grouped bsAbs into two main classes based on their mechanism of action, namely cell-bridging bsAbs and antigen-crosslinking bsAbs (non-cell-bridging molecules; Labrijn et al, 2019). Most cell-bridging bsAbs are designed for cancer treatment by linking immune cells to malignant cells. Through sequential binding, that is, by binding the cancer cell first owing to a higher affinity to tumor antigens, cell-bridging bsAbs can improve specificity and effectiveness with reduced non-specific side effects and lower dosage compared with mAbs. In contrast, antigen-crosslinking bsAbs target two antigens or two receptors simultaneously. Their main MoA is either blocking signals of cell growth/survival or activation of immune cells (Engelman et al, 2007). Antigen-crosslinking bsAbs basically act similar to mAbs except that they bind two different targets.

In an aspect, the present disclosure provides the use of an immunotherapeutic protein as described herein, for treating or preventing a disease or condition in a subject, wherein the disease or condition may be selected from, for example, autoimmune diseases and conditions, cardiovascular disease, neurodegenerative disease, other inflammatory diseases, transplantation condition or rejection, infectious diseases and proliferative diseases.

In an aspect, the present disclosure provides the use of an immunotherapeutic protein as described herein, in the manufacture of a medicament for treating or preventing a disease or condition, wherein the disease or condition is selected from autoimmune diseases and conditions, cardiovascular disease, neurodegenerative disease, other inflammatory diseases, transplantation condition or rejection, infectious diseases and proliferative diseases.

In an aspect, the present disclosure provides a method for treating or preventing a disease or condition, comprising administering to the subject an effective amount of an immunotherapeutic protein as described herein, wherein the disease or condition is selected from autoimmune diseases and conditions, cardiovascular disease, neurodegenerative disease, other inflammatory diseases, transplantation condition or rejection, infectious diseases and proliferative diseases.

Among the diseases and conditions that may be treated or prevented by the method as described herein, are autoimmune diseases and conditions such as SLE and MS, other inflammatory diseases (e.g. immune complex vasculitis), infectious diseases and proliferative diseases (especially solid tumours such as breast cancers), blood cancers such as lymphoproliferative disorders (LPDs) including leukaemias (e.g. acute lymphoblastic leukaemia (ALL) and chronic lymphocytic leukaemia (CLL)), adenocarcinomas and lymphomas, as well as multiple myeloma (MM) and X-linked proliferative disease.

The method as described herein will be typically applied to the treatment of a disease or condition in a human subject. However, the subject may also be selected from, for example, livestock animals (e.g. cows, horses, pigs, sheep and goats), companion animals (e.g. dogs and cats) and exotic animals (e.g. non-human primates, tigers, elephants etc).

In some embodiments, wherein the immunotherapeutic protein comprises a fusion protein comprising a partner polypeptide linked to an Fc region component comprising at least a CH3 domain (or at least a constant heavy domain 4 (CH4) domain) and said partner polypeptide is a cell surface receptor polypeptide (or a fragment thereof) that is capable of binding to a structural protein of a virus such that the immunotherapeutic protein may act as a “decoy” to block viral interaction and cellular entry of a virus to a host cell, the method as described herein may further comprise administering an antibody directed against the said virus (i.e. target virus). For example, where the immunotherapeutic protein comprises an ACE2 polypeptide (or a fragment thereof) which binds with the RBD of the CoV-2 spike protein, the antibody may be selected from antibodies that are, for example, broadly neutralising coronavirus mAbs (ie bNmAbs which can neutralise multiple coronavirus types or strains), broadly reactive coronavirus mAbs (ie mAbs which may not be neutralising but can bind with multiple coronavirus types or strains), broadly neutralising SARS-CoV-2 mAbs (ie bNmAbs which can neutralise multiple SARS-CoV-2 strains) and broadly reactive SARS-CoV-2 mAbs, broadly neutralising coronavirus spike stem specific mAbs, broadly reactive coronavirus spike stem specific mAbs, are broadly neutralising SARS-CoV-2 spike stem specific mAbs, and broadly reactive SARS-CoV-2 spike stem specific mAbs. In some specific embodiments, the antibody may be, for example, targeted to an epitope of a SARS-CoV-2 structural protein other than the spike protein (S) such as the envelope protein (E), the membrane protein (M) or the nucleocapsid protein (N). In some other specific embodiments, the antibody may be targeted to, for example, an epitope on the spike protein (S) but at a site distinct from the RBD. More generally, where the immunotherapeutic protein comprises a cell surface receptor polypeptide other than an ACE2 polypeptide, or a co-receptor polypeptide, or a fragment thereof, the antibody directed against the target virus may be selected from antibodies that are, for example, broadly neutralising against a class/family of viruses (e.g. human immunodeficiency viruses (HIV)), or which are broadly reactive against a class/family of viruses, broadly neutralising of strains of a specific virus type (e.g. bNmAbs which can neutralise multiple HIV-1 strains), and mAbs which are broadly reactive to a specific virus type. As shown hereinafter, it has been found that an immunotherapeutic protein of the present disclosure and an antibody directed against the target virus may synergistically cooperate to enhance CDC killing of cells (e.g. virus-infected cells).

Similarly, where the immunotherapeutic protein comprises a fusion protein comprising a partner polypeptide linked to an Fc region component comprising at least a CH3 domain (or at least a constant heavy domain 4 (CH4) domain) and said partner polypeptide is, for example, directed at a target of therapeutic significance (e.g. the partner polypeptide is a cell surface receptor polypeptide (or a fragment thereof) that is capable of binding to CTLA4 which functions as an immune checkpoint and downregulates immune responses), the method as described herein may further comprise administering an antibody directed against the said target (e.g. an antibody which binds to CTLA4) to, for example, provide an enhanced response such as enhanced CDC killing of cells (e.g. cancer cells).

Where the method as described herein does comprise administering an antibody in addition to a fusion protein (i.e. as described in the preceding two paragraphs), preferably the antibody comprises an Fc region component comprising an amino acid substitution at the position corresponding to H429 of the amino acid sequence of the human IgG1 heavy chain polypeptide (Eu numbering) such as, for example, an H429X² amino acid substitution, where X² is selected from phenylalanine (H429F), glutamate (H429E), glutamine (H429Q),serine (H429S), alanine (H429A), threonine (H429T), tyrosine (H429Y), leucine (H429L), valine (H429V), glycine (H429G), tryptophan (H429W), arginine (H429R) and proline (H429P).

In an aspect, the present disclosure provides a pharmaceutical composition or medicament comprising an immunotherapeutic protein as defined herein, and a pharmaceutically acceptable carrier, diluent and/or excipient.

The immunotherapeutic protein may be administered in combination with one or more additional agent(s) for the treatment of the particular disease or condition being treated. For example, in the context of treating proliferative diseases, the immunotherapeutic protein may be used in combination with other agents for treating cancer (including, for example, antineoplastic drugs such as cisplatin, gemcitabine, cytosine arabinoside, doxorubicin, epirubicin, taxoids including taxol, topoisomerase inhibitors such as etoposide, cytostatic agents such as tamoxifen, aromatase inhibitors (e.g. as anastrozole) and inhibitors of growth factor function (e.g. antibodies such as the antierbB2 antibody trastuzumab (Herceptin™). In some embodiments, the immunotherapeutic protein may be administered with one or more additional agent(s) which may also be an immunotherapeutic protein(s) according to the present disclosure. For example, where a first immunotherapeutic protein according to the present disclosure comprises an ACE2 polypeptide (or a fragment thereof) which binds with the RBD of the CoV-2 spike protein, then the second immunotherapeutic protein of the present disclosure may be an antibody directed against the target CoV-2 virus, particularly one targeted to an epitope of a different structural protein (e.g. the envelope protein (E), the membrane protein (M) or the nucleocapsid protein (N)) or on the same structural protein (i.e. the spike protein (S)) but at a site distinct from the RBD. As shown hereinafter, it has been found that a combination of such immunotherapeutic proteins may synergistically cooperate to enhance CDC killing of cells (e.g. virus-infected cells).

Where used in combination with other agents, the immunotherapeutic protein can be administered in the same pharmaceutical composition or in separate pharmaceutical compositions. If administered in separate pharmaceutical compositions, the immunotherapeutic protein and the other agent(s) may be administered simultaneously or sequentially in any order (e.g. within seconds or minutes or even hours (e.g. 2 to 48 hours)).

The immunotherapeutic protein may be formulated into a pharmaceutical composition with a pharmaceutically acceptable carrier, diluent and/or excipient. Examples of suitable carriers and diluents are well known to those skilled in the art, and are described in, for example, Remington's Pharmaceutical Sciences, Mack Publishing Co., Easton, PA 1995. Examples of suitable excipients for the various different forms of pharmaceutical compositions described herein may be found in the Handbook of Pharmaceutical Excipients, 2^{nd} Edition, (1994), Edited by A Wade and PJ Weller. Examples of suitable carriers include lactose, starch, glucose, methyl cellulose, magnesium stearate, mannitol, sorbitol and the like. Examples of suitable diluents include ethanol, glycerol and water. The choice of carrier, diluent and/or excipient may be made with regard to the intended route of administration and standard pharmaceutical practice.

A pharmaceutical composition comprising an immunotherapeutic protein as defined herein may further comprise any suitable binders, lubricants, suspending agents, coating agents and solubilising agents. Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilising agents, and even dyes may be provided in the pharmaceutical composition. Examples of preservatives include sodium benzoate, sorbic acid and esters of phydroxybenzoic acid. Anti-oxidants and suspending agents may be also used.

A pharmaceutical composition comprising an immunotherapeutic protein as defined herein will typically be adapted for intravenous or subcutaneous administration. As such, a pharmaceutical composition may comprise solutions or emulsions which may be injected into the subject, and which are prepared from sterile or sterilisable solutions. A pharmaceutical composition may be formulated in unit dosage form (i.e. in the form of discrete portions containing a unit dose, or a multiple or sub-unit of a unit dose).

The immunotherapeutic protein, uses and pharmaceutical composition of the present disclosure are hereinafter further described with reference to the following, non-limiting examples.

### EXAMPLES

### Example 1 ACE-2-Fc Fusion Proteins Comprising an H429 Mutation and Activity Analysis

### Methods and Materials

### Constructs and Fusion Proteins

The amino acid sequence of the human ACE2 polypeptide is available from the European Nucleotide Archive (ENA, European Molecular Biology Laboratory) at accession no. BAB40370. The ectodomain of the protein (amino acids 19 to 740; shown as SEQ ID NO: 1 in Table 1) is comprised of a catalytic domain and a collectrin domain. Different forms of the ACE2 ectodomain were produced and studied in this example; particularly, a truncated (tr) ACE2 ectodomain comprising amino acids 19 to 615 of the mature ACE2 polypeptide (named trACE2 shown as SEQ ID NO: 2 in Table 1) and excluding the collectrin domain, a full length (fl) ACE2 ectodomain to (flACE2; comprising amino acids 19 to 740 of the mature ACE2 polypeptide; SEQ ID NO: 1 in Table 1), and an enhanced flACE2 ectodomain (EflACE2) comprising a triple mutation within the ACE2 polypeptide that has been reported to improve its binding affinity to the S protein (Chan et al., 2020 supra). These proteins were produced as fusion proteins with an Fc region component derived from human IgG1 to generate trACE2-Fc, flACE2-Fc and EflACE2-Fc according to standard techniques (see Table 2).

For example, a construct encoding the trACE2 ectodomain in pcDNA3.4 (Thermo Fisher Scientific) was prepared by joining a polynucleotide sequence encoding the trACE2 ectodomain to a synthetic sequence encoding a linker and a sequence encoding human IgG1 Fc (particularly, IgG1 Fc having the amino acid sequence of accession no. AXN93652.1 (immunoglobulin gamma 1 constant region, partial [Homo sapiens]; National Center for Biotechnology Information (NCBI) database). For the generation of a flACE2-Fc expression construct, a KpnI digestion of the trACE2 construct was conducted followed by insertion of a codon-optimised polynucleotide sequence encoding the ACE2 collectrin domain (GeneArt, Thermo Fisher Scientific). For the generation of an EflACE2-Fc expression construct, a synthetic polynucleotide sequence equivalent to that encoding flACE2-Fc, but with three mutations (i.e. T27Y, L79T and N330Y; sACE2.v2.4 described by Chan et al., 2020 supra) was used. In addition, variants of the fusion proteins were produced that incorporated H429F and H429Y mutations in the Fc component introduced using cleavage at a unique AfeI site within the IgG Fc-encoding sequence, and the subsequent insertion of appropriate mutagenic oligonucleotides using NEBuilder (New England Biolabs, Ipswich, MA, United States of America) according to the manufacturer's instructions.

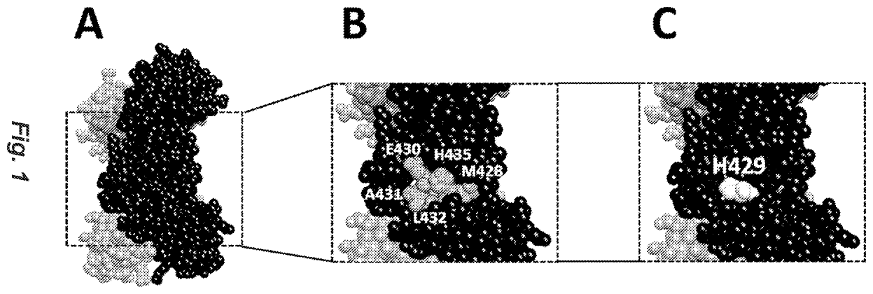

The H429 residue occupies a “buried” site within the IgG1 Fc structure (see FIG. 1) and is occupied by a histidine (His/H) residue, which is also found in the corresponding position of the Fc fragment of all other human immunoglobulin classes (FIGS. 3, 4), and accordingly is found in the corresponding position of, for example, human IgG1, IgG2, IgG3 and IgG4, as well as primate IgG subclasses and some mouse IgG subclasses. For example, the structure published in 1981 of human IgG1-Fc (PDB:1Fc1; Deisenhofer, 1981 supra) indicates that H429 is not a surface accessible residue (FIG. 1), and in a space-filled representation of the Fc, H429 is only “visible” when overlaying residues are rendered in a non-space filling manner, as shown in FIG. 1C. Also, analysis of residues neighbouring H429 indicates that H429 lies beneath the side-chains of these residues and calculation of accessible surface area (ASA) for the Fc residues indicates that H429 is solvent inaccessible (0% ASA (Å²) FIG. 1C). H429 is also not a surface accessible residue in other Fc structures including the Fc within the structure of the anti-HIV mAb (PDB:1IHZH; Saphire et al., supra) which forms a hexameric ring structure, but H429 does not form part of the interface. Similarly, analysis of the Fc structure of the anti-Lewis Y mAb (Ramsland et al., J Immunol 187(6):3208-321′7, 2011) showed that the H429 is also buried in this antibody.

```
TABLE 1
SEQ ID NO: 1
                                      20 30 40 50 60 70 80
                                ETGST IEEQAKTFLD KFNHEAEDLF YQSSLASWNY NTNITEENVQ NMNNAGDKWS AFLKEQSTLA
                90 100 110 120 130 140 150 160
QMYPLQEIQN LTVKLQLQAL QQNGSSVLSE DKSKRINTIL NTMSTIYSTG KVCNPDNPQE CLLLEPGLNE IMANSLDYNE
              170 180 190 200 210 220 230 240
RLWAWESWRS EVGKQLRPLY EEYVVLKNEM ARANHYEDYG DYWRGDYEVN GVDGYDYSRG QLIEDVEHTF EEIKPLYEHL
              250 260 270 280 290 300 310 320
HAYVRAKLMN AYPSYISPIG CLPAHLLGDM WGREWTNLYS LTVPFGQKPN IDVTDAMVDQ AWDAQRIFKE AEKFFVSVGL
              330 340 350 360 370 380 390 400
PNMTQGFWEN SMLTDPGNVQ KAVCHPTAWD LGKGDFRILM CTKVTMDDFL TAHHEMGHIQ YDMAYAAQPF LLRNGANEGE
              410 420 430 440 450 460 470 480
HEAVGEIMSL SAATPKHLKS IGLLSPDFQE DNETEINFLL KQALTIVGTL PFTYMLEKWR WMVFKGEIPK DQWMKKWWEM
              490 500 510 520 530 540 550 560
KREIVGVVEP VPHDETYCDP ASLFHVSNDY SFIRYYTRTL YQFQFQEALC QAAKHEGPLH KCDISNSTEA GQKLENMLRL
              570 580 590 600 610 620 630 640
GKSEPWTLAL ENVVGAKNMN VRPLLNYFEP LFTWLKDQNK NSFVGWSTDW SPYADQSIKV RISLKSALGD RAYEWNDNEM
              650 660 670 680 690 700 710 720
YLFRSSVAYA MRQYFLKVKN QMILFGEEDV RVANLKPRIS FNFFVTAPKN VSDIIPRTEV EKAIRMSRSR INDAFRLNDN
              730 740 750 760 770 780 790 800
                                                      Fc (Eu numbering)
SLEFLGIQPT LGPPNQPPVS GGGGSTHTCP PCPAPELLGG PSVFLFPPKP KDTLMISRTP EVTCVVVDVS HEDPEVKENW
              810 820 830 840 850 860 870 880
YVDGVEVHNA KTKPREEQYN STYRVVSVLT VLHQDWLNGK EYKCKVSNKA LPAPIEKTIS KAKGQPREPQ VYTLPPSREE
              890 900 910 920 930 940 950 960
MTKNQVSLTC LVKGFYPSDI AVEWESNGQP ENNYKTTPPV LDSDGSFFLY SKLTVDKSRW QQGNVFSCSV MHEALHNHYT
970
QKSLSLSPGK
Comments: SEQ ID NO: 1 is the predicted sequence of the mature “flACE2-Fc” fusion protein. The full
length (fl) ACE2 ectodomain comprises the catalytic domain and the collectrin (or neck) domain and
sequence proximal to the transmembrane region. The N-terminus sequence ETG is predicted to remain
following cleavage of the following underlined leader sequence (MGILPSPGMPALLSLVSLLS
VLLMGCVAETG; SEQ ID NO: 178) upon secretion. The numbering above the fusion protein sequence
utilises the numbering of ACE2, amino acid residues S19 to S740, then a linking sequence (GGGGS; SEQ
ID NO: 179), followed by a human IgG1 Fc comprising T223-K447 (according to the Eu numbering
convention, labeled immediately above the C-terminus Fc sequence) and includes the CH2 and CH3
domains. The IgG1 Fc amino acid, H429, is shown in bold and underlined (Eu numbering). The ACE2
ectodomain sequence matches that of Accession no. BAB40370. Also, a variant immunotherapeutic
protein “EflACE2-Fc” was constructed whereby, the amino acids at positions 27, 79 and 330 were
mutated to enhance binding affinity to CoV-2 S as follows: T27Y, L79T and N330Y (Chan et al., 2020
supra). The IgG1 Fc sequence matches the immunoglobulin gamma 1 constant region, partial [Homo
sapiens] sequence of Accession no. AXN93652.1.
SEQ ID NO: 2
                                      20 30 40 50 60 70 80
                                ETGST IEEQAKTFLD KFNHEAEDLF YQSSLASWNY NTNITEENVQ NMNNAGDKWS AFLKEQSTLA
                90 100 110 120 130 140 150 160
QMYPLQEIQN LTVKLQLQAL QQNGSSVLSE DKSKRLNTIL NTMSTIYSTG KVCNPDNPQE CLLLEPGLNE IMANSLDYNE
                170 180 190 200 210 220 230 240
RLWAWESWRS EVGKQLRPLY EEYVVLKNEM ARANHYEDYG DYWRGDYEVN GVDGYDYSRG QLIEDVEHTF EEIKPLYEHL
              250 260 270 280 290 300 310 320
HAYVRAKLMN AYPSYISPIG CLPAHLLGDM WGREWTNLYS LTVPFGQKPN IDVTDAMVDQ AWDAQRIFKE AEKFFVSVGL
              330 340 350 360 370 380 390 400
PNMTQGFWEN SMLTDPGNVQ KAVCHPTAWD LGKGDFRILM CTKVTMDDEL TAHHEMGHIQ YDMAYAAQPF LLRNGANEGE
              410 420 430 440 450 460 470 480
HEAVGEIMSL SAATPKHLKS IGLLSPDFQE DNETEINFLL KQALTIVGTL PFTYMLEKWR WMVFKGEIPK DQWMKKWWEM
              490 500 510 520 530 540 550 560
KREIVGVVEP VPHDETYCDP ASLFHVSNDY SFIRYYTRTL YQFQFQEALC QAAKHEGPLH KCDISNSTEA GQKLENMLRL
              570 580 590 600 610 620 630 640
                                                                                                                                                  230 240
GKSEPWTLAL ENVVGAKNMN VRPLLNYFEP LFTWLKDQNK NSFVGWSTDW SPYADGSGSG SGTHTCPPCP APELLGGPSV
              650 660 670 680 690 700 710 720
              250 260 270 280 290 300 310 320
FLFPPKPKDT LMISRTPEVT CVVVDVSHED PEVKENWYVD GVEVHNAKTK PREEQYNSTY RVVSVLTVLH QDWLNGKEYK
              730 740 750 760 770 780 790 800
              330 340 350 360 370 380 390 400
CKVSNKALPA PIEKTISKAK GQPREPQVYT LPPSREEMTK NQVSLTCLVK GFYPSDIAVE WESNGQPENN YKTTPPVLDS
              810 820 830 840 847
              410 420 430 440 447
DGSFFLYSKL TVDKSRWQQG NVFSCSVMHE ALHNHYTQKS LSLSPGK
Comments: SEQ ID NO: 2 is the predicted sequence of the mature “trACE2-Fc” fusion protein. The N-
terminus sequence ETG is predicted to remain following cleavage of the following underlined leader
sequence (MGILPSPGMPALLSL VSLLSVLLMGCVAETG; SEQ ID NO: 178) upon secretion. The
numbering above the fusion protein sequence utilises the numbering of ACE2, amino acid residues S19 to
D615, then a linking sequence (GSGSGSG; SEQ ID NO: 179), followed by a human IgG1 Fc, and which,
according to the Eu numbering convention, comprises T223-K447. The IgGI Fc amino acid, H429, is
shown bold and underlined. The truncated ACE2 ectodomain sequence matches that of Accession no.
BAB40370. The IgG1 Fc sequence matches the immunoglobulin gamma 1 constant region, partial [Homo
sapiens] sequence of Accession no. AXN93652.1.
```

Expression of the fusion proteins was conducted using transient transfection of Expi293 cells (Thermo Fisher Scientific). All expressed fusion proteins were first purified from the culture supernatant by ion exchange chromatography (IEX) followed by additional purification by size exclusion chromatography (SEC). Particularly, the supernatant of the Expi293 cells transiently transfected for the expression of the respective ACE2-Fc fusion protein (where the Fc region was according to the wild type (WT) hIgG1 Fc mentioned in the preceding paragraph) was extensively dialysed against 10 mM Tris-HCl pH 8.0 and applied to a High-Q column (BioRad Laboratories, Hercules, CA, United States of America). Bound proteins were eluted with a linear gradient to buffer A containing 0.4 M NaCl. Fractions were examined by SDS-PAGE, and those fractions containing the flACE2-Fc WT fusion protein were pooled and concentrated using a 30 kDa cut-off filtration device (Pall Corporation, Port Washington, NY, United States of America) and separated by size exclusion chromatography (SEC) using a Superose 6 column (GE Life Sciences, Chicago, IL, United States of America).

The SARS-CoV-2 RBD-Ig and RBD AviTag have been described previously. The RBD AviTag was biotinylated in situ using Expi293BirA cells (Wines B D et al., J Immunol 197(4):1507-1516, 2016).

Lamelli native PAGE (N-PAGE), 150V, 2.5 h, 4 °C, was according to Wines B D et al., J Immunol 162(4):2146-2153, 1999).

### Virus Neutralisation Assays

Antiviral titre was determined using SARS-CoV-2 (CoV/Australia/VIC01/2020) in a microneutralisation assay as described previously (Juno J A et al., Nat Med 26(9):1428-1434, 2020).

### Bio-Layer Interferometry

Measurements of the affinity of the ACE2-Fc fusion proteins for the CoV-2 S RBD were performed on the Octet RED96e (FortdBio, Fremont, CA, United States of America). All assays were performed at 25 °C using anti-human IgG Fc capture (AHC) biosensor tips (FortdBio) in kinetics buffer (phosphate buffered saline (PBS) pH 7.4 supplemented with 0.1% (w/v) bovine serum albumin (BSA) and 0.05% (v/v) TWEEN-20). After a 60 second (60s) biosensor baseline step, the fusion proteins (20 mg/mL) were loaded onto anti-human IgG Fc capture (AHC) biosensors by submerging sensor tips for 200s and then washing in kinetics buffer for 60s. For most of the fusion proteins, association measurements were performed by dipping into a two-fold dilution series of SARS-CoV-2 RBD from 16-250 or 500 nM for 180s and then measuring dissociation in kinetics buffer for 180s. For EflACE2-Fc WT, a two-fold dilution series of 2-31 or 63 nM was used. The biosensor tips were regenerated five times using a cycle of 5s in 10 mM glycine pH 1.5 and 5s in kinetics buffer, and baseline drift was corrected by subtracting the average shift of a fusion protein-loaded sensor not incubated with SARS-CoV-2 RBD, and an unloaded sensor incubated with SARS-CoV-2 RBD. Curve fitting analysis was performed with Octet Data Analysis 10.0 software using a global fit 1:1 model to determine K_{D} values and kinetic parameters. Curves that could not be fitted were excluded from the analyses. Kinetic constants reported were representative of two independent experiments.

ACE2-Fc Fusion Proteins and Recombinant Dimeric rsFcγR Binding by Flow Cytometry

The ACE2-Fc fusion proteins or rituximab (a chimeric mAb targeted to CD20), at 5 μg/ml, or the indicated concentrations were incubated with Ramos cells expressing transfected spike protein (Ramos-S cells; Lee W S et al., medRxiv doi:10.1101/2020.12.13.20248143, 2020) at 5×10⁶ cells/ml in 25 μl of PBS containing 0.5% (w/v) BSA and 1 mM glucose (PBS/BSA/G), for 30 minutes on ice, and then washed twice with PBS/BSA/G, incubated with PE or FITC conjugated anti-human IgG-Fc for 30 minutes on ice, before being washed again and resuspended in 25 μl of PBS/BSA/G.

Evaluation of binding of dimeric recombinant soluble FcγR (rsFcγR) was performed as previously described (Wines et al., 2016 supra). The ACE2-Fc opsonised Ramos-S cells or rituximab-opsonised cells were resuspended in 0.5 μg/ml of biotinylated dimeric rsFcγRIIa (H131 allelic form) or dimeric rsFcγRIIIa (V158 allelic form) or BSA/PBS/G and incubated for 30 minutes on ice followed by 1/500 streptavidin-APC (or anti-hIgG-Fc FITC for confirmation of ACE2-Fc opsonisation) for 20 minutes on ice. The cells were washed, resuspended in PBS/BSA/G and analysed on a Canto™ II flow cytometer (Becton Dickinson, Franklin Lakes, NJ, United States of America).

### Complement Dependent Cytotoxicity (CDC) Assay

CDC was measured using Ramos-S cells opsonised with ACE2-Fc function proteins or the control mAb, rituximab, then incubation with human serum as a source of complement. Thus, Ramos-S cells were first incubated with a fusion protein or rituximab as above (5×10⁶ cells/ml in 25 μl of PBS/BSA/G for 30 minutes on ice), then washed before resuspending in 1/3 diluted normal human serum for 30 minutes at 37 °C Cells were washed twice with PBS and the dead cells were then enumerated by staining with 1/500 Zombie Green (from a Zombie Green Fixable Viability kit according to the manufacturer's instruction, BioLegend, San Diego, CA, United States of America) before fixing with 2% paraformaldehyde in PBS and analysis on a Canto™ II flow cytometer (Becton Dickinson).

### Complement Fixation Immunoassay for ACE2-Fc Fusion Proteins

Ninety-six well flat-bottom MaxiSorp Nunc plates (Thermo Fisher Scientific) were coated with 5 μg/ml Avidin in PBS overnight, blocked, and then incubated with either a serial two-fold dilution or single concentration (2.5 μg/ml) of biotinylated RBD (Hartley et al., Science Immunology 5(54) doi:10.1126/sciimmunol.abf8891, 2020) in 0.1% casein for 1 hour at RT. The ACE2-Fc fusion proteins were then added over the indicated concentration range. To measure C1q fixation, the plates were incubated with 10 μg/ml purified human C1q (Merck Millipore, Burlington, MA, United States of America) for 30 minutes at RT followed by 1/2000 dilution of rabbit anti-C1q IgG (Kurtovic L et al., BMC Med 17:45 2019) for 1 hour at RT. In experiments to measure C5b-C9 fixation, the plates were incubated with 10% fresh human serum for 30 minutes at RT followed by 1/2000 dilution of rabbit anti-C5b-C9 (Merck Millipore) for 1 hour at RT washed and then incubated with goat anti-rabbit IgG conjugated to HRP (Merck Millipore) at 1/2000 dilution for 1 hour at RT followed by TMB substrate (Life Technologies Corporation, Carlsbad, CA, United States of America) for 15-20 minutes at RT. Reactivity was stopped using 1 M sulfuric acid and absorbance was measured at OD450 nm. Test samples and reagents were prepared in PBS supplemented with 0.1% (w/v) casein and plates washed three times between each step using PBS containing 0.05% (v/v) TWEEN-20. Samples were tested in duplicate and corrected for background reactivity using negative control wells from which ACE2-Fc proteins were omitted. The mean and SEM from independent experiments are shown.

### RBD Variant Multiplex Assay

A custom multiplex array was produced with the SARS-1 S1 subunit (ACROBiosystems, Newark, DE, United States of America), SARS-Cov-2 S1 and HCoV NL63 S1 and S2 subunits (Sino Biological Inc., Beijing, China), NL63 S trimer (BPS Bioscience, San Diego, CA, United States of America), and a hexahistidine-tagged RBD WT protein (amino acids 19-613) and 21 variants identified from the GISAID RBD surveillance repository that had been expressed from pcDNA3 in Expi293 cells and purified by affinity chromatography. Bead coupling, washing steps, and data acquisition on a FlexMap3D™ analyser (Luminex Corporation, Austin, TX, United States of America) was as previously described (Lee et al., 2020 supra). Briefly, direct binding of the ACE2-Fc fusion proteins trACE2-Fc, flACE2-Fc and EflACE2-Fc at 0.5 to 250 nM, were detected using 25 μl of 1.3 μg/ml anti-human IgG R-Phycoerythrin Conjugate (SouthernBiotech, Birmingham, AL, United States of America). Data were fitted as 3-parameter agonist versus response curves (r²>0.85) to determine each EC₅₀. In a competition assay, RBD- or S1-coupled beads were simultaneously incubated with 20 μl of 25 μg/ml of biotinylated, AviTagged ACE2 in the presence of the various unbiotinylated ACE2-Fc fusion protein “competitors”, at 1 to 280 nM for 2 hour at RT. The binding of biotinylated ACE2 (aa 19 to 615) was performed using, first, Streptavidin, R-Phycoerythrin Conjugate (SAPE) (Thermo Fisher Scientific) at 4 μg/ml (1 hour), followed by 10 μg/ml of R-Phycoerythrin, Biotin-XX Conjugate (Thermo Fisher Scientific) (1 hour).

### FcγRIIIa-NF-κB-RE Nanoluciferase Reporter Assay

FcγRIIIa-NF-κB-RE nanoluciferase reporter assays were conducted using IIA1.6/FcR-γ/FcγRIIIa V158 cells expressing a NF-κB response element-driven nanoluciferase (NanoLuc, pNL3.2.NF-κB-RE[NlucP/NF-κB-RE/Hygro], Promega Corporation, Madison, WI, United States of America), and was performed essentially as previously described (Lee et al., 2020 supra). Briefly, Ramos cells expressing Spike-IRES-orange2 were used as target cells and were incubated with agonists and the FcγRIIIa/NF-κB-RE reporter cells for 5 hours before measurement of induced nanoluciferase with Nano-Glo substrate (Promega Corporation).

### Results and Discussion

### ACE2-Fc Fusion Protein Construction and Production

A series of ACE2-Fc fusion proteins (Table 2) were produced and analysed for their capacity to neutralise SARS-CoV-2 infection and mediate Fc-dependent effector functions normally attributed to the mechanisms of action of antibodies.

TABLE 2

| | | | IgG1 Fc |
|---|---|---|---|
| | | ACE2 | mutation/ |
| | ACE2 ectodomain | mutation/ | modification |
| Protein name | form (sequence) | modification | (Eu numbering) |
| trACE2 | Truncated ACE2 | Not mutated | N/A* |
| | (amino acids 19-615) | | |
| trACE2-Fc WT | Truncated ACE2 | Not mutated | Not modified |
| | (amino acids 19-615) | | |
| trACE2-Fc-H429F | Truncated ACE2 | Not mutated | His 429 Phe |
| | (amino acids 19-615) | | |
| trACE2-Fc-H429Y | Truncated ACE2 | Not mutated | His 429 Tyr |
| | (amino acids 19-615) | | |
| trACE2-Fc-kif | Truncated ACE2 | Not mutated/modified | Modified glycan |
| | (amino acids 19-615) | N-glycans | at Asn 297 |
| f1ACE2-Fc WT | Full length ACE2 | Not mutated | Not modified |
| | (amino acids 19-740) | | |
| f1ACE2-Fc-H429F | Full length ACE2 | Not mutated | His 429 Phe |
| | (amino acids 19-740) | | |
| flACE2-Fc-H429Y | Full length ACE2 | Not mutated | His 429 Tyr |
| | (amino acids 19-740) | | |
| EflACE2-Fc WT | Full length ACE2 | Thr 27 Tyr | Not modified |
| | (amino acids 19-740) | Leu 79 Thr | |
| | | Asn 330 Tyr | |
| EflACE2-Fc-H429F | Full length ACE2 | Thr 27 Tyr | His 429 Phe |
| | (amino acids 19-740) | Leu 79 Thr | |
| | | Asn 330 Tyr | |
| EflACE2-Fc-H429Y | Full length ACE2 | Thr 27 Tyr | His 429 Tyr |
| | (amino acids 19-740) | Leu 79 Thr | |
| | | Asn 330 Tyr | |

*N/A: Not applicable as no Fc present (i.e. truncated ectodomain only)

In an attempt to enhance the avidity of binding to the SARS-CoV-2 spike protein or to confer and improve Fc-dependent effector functions, three versions of the ACE2 ectodomain were fused to an Fc region of IgG1 either unmodified or altered by mutation (i.e. substitution of histidine 429 with phenylalanine (H429F) or tyrosine (H429Y)) or modified glycosylation (i.e. lacking core fucose; trACE2-Fc-kif).

### ACE2-Fc Fusion Protein Purification

Proteins were produced in Expi293 cells and purified by (an)ion exchange (IEX) then by size exclusion chromatography (SEC) at pH 7.4 (FIG. 5). All of the fusion proteins showed a similar IEX purification profile to that of flACE2-Fc-WT (FIG. 5A) which contained a major elution peak (peak *; FIG. 5A), and SDS-PAGE analysis of the collected fractions showed a single major species of about 270 kDa (FIG. 5B). Except for those fusion proteins comprising the H429Y mutation, SEC analysis and purification confirmed the presence of a major monomeric species (FIG. 5C) (NB. The monomer species are considered to be single molecules (i.e. monomer molecules) comprising two (dimerised) copies of the respective ACE2-Fc fusion protein) which were collected for further analysis, and only trace quantities of higher molecular weight oligomers and other impurities were apparent for example for flACE2-Fc-WT (FIG. 5). Indeed, the presence of a similar monomeric species was also evident following the IEX purification of all fusion proteins with unmodified Fc-WT or an Fc region component including an H429F modification (not shown). For the fusion proteins comprising the H429Y mutation, SEC analysis (FIG. 5D) (i.e. of the major IEX peak from flACE2-Fc H429Y mutant, not shown), and also of the major IEX peaks from the trACE2-Fc H429Y and EflACE2-Fc H429Y proteins (not shown)), revealed the presence of significant quantities of oligomeric (Yₒₗᵢ) and monomeric (Yₘₙ) species, which were subsequently evaluated separately for functional activity.

### Binding of ACE2-Fc Fusion Proteins RBD of SARS-CoV-2 S by ELISA

The binding of the trACE2-Fc WT, flACE2-Fc WT and EflACE2-Fc WT fusion proteins to SARS-CoV-2 receptor binding domain (RBD) (FIG. 6A-C) were similar overall (i.e. EC₅₀ 0.35 nM, 0.27 nM and 0.25 nM respectively; FIG. 6D) with the enhanced intrinsic affinity of the EflACE2-Fc being less apparent with binding to this bivalent form of RBD, which was presented in fusion with mouse IgG1 Fc (RBD-Ig). The binding activity of the various fusion proteins with mutated Fc components were also equivalent, excepting the monomeric flACE2-Fc-H429Y which was of slightly lower affinity (EC₅₀ 0.48 nM) (FIG. 6D) and the other H429Y Fc variants likewise trending to a lower level of affinity (FIG. 6).

### Oligomerisation of the ACE2-Fc H429Y Fusion Protein is pH Dependent

Oligomerisation of fusion proteins including a mutated H429Y Fc component was examined by SEC separation at pH 5.0 of the flACE2-Fc-H429Y prepared by IEX. In contrast to SEC at pH 7.4 (Figure and FIG. 7A), SEC at pH 5.0 (FIG. 7B) revealed a greater proportion of monomer Yₘₙ which N-PAGE showed was purified to homogeneity (FIG. 7C: lane 1 cf lane 2). The purified monomeric flACE2-Fc-H429Yₘₙ, pH 5.0, was re-analysed by dialysis at pH 7.4 followed by both N-PAGE (FIG. 7C) and by SEC at pH 7.4 (FIG. 7D). Re-exposure to pH 7.4 yielded a mix of oligomer Yₒₗᵢ and monomer Yₘₙ species (FIG. 7C; lane 2 pH 5.0 cf lane 5 pH 7.4) indicating some equilibrium between these forms occurs at neutral pH. The flACE2-Fc-H429Yₘₙ prepared at pH 5.0 showed equivalent binding to RBD-Ig as the other flACE2-Fc WT fusion proteins and Fc variants (FIG. 7E). It is also clear that the prevalence of oligomers is related to the tyrosine substitution of histidine 429 as this was not observed in the fusion proteins with phenylalanine-substituted H429F Fc region component.

### Evaluation of Virus Neutralisation Potency

The antiviral activities of the ACE2-Fc fusion proteins was determined in a micro-neutralisation assay of SARS-CoV-2 infection of Vero cells (FIG. 8) where the EC₅₀ endpoint corresponds to neutralisation of ~99% of the inoculum virions (Khoury D S et al., Nat Rev Immunol 20(12):727-738, 2020).

The SARS-CoV-2 neutralisation endpoint of the truncated ectodomain, trACE2 (2.70 μM), was improved ~10-fold by fusion to the wild type Fc region of IgG1 (trACE2-Fc WT, 283 nM) (FIG. 8). The improved potency is consistent with improved avidity of binding to SARS-CoV-2 spike RBD because of ACE2-Fc bivalency resulting from fusion of ACE2 ectodomains to the IgG Fc region and was also similar to that of flACE2-Fc WT. The EflACE2-Fc-WT showed a further ~20-fold improvement (11 nM) over the flACE2-Fc WT and trACE2-Fc WT fusion proteins and ~200-fold more than un-fused trACE2.

Analysis of the Fc modifications revealed several interesting differences. First, the H429Y mutation in trACE2-Fc and flACE2-Fc improved their virus neutralisation potency (FIG. 8); this was surprising as the neutralisation function of the ACE2-Fc fusion proteins occurs well away from the modifications at the H429 position (i.e. in the fusion proteins, the ACE2 polypeptide is expected to neutralise the virus, but the H429 residue is distal to the ACE2 being present in the CH3 domain of the Fc component). Secondly, it was found that the neutralisation activity (21.9 nM) of the oligomeric form of trACE2-Fc-H429Yₒₗᵢ, isolated by SEC at pH 7.4 was enhanced 13-fold over the trACE2-Fc WT. Both the oligomer, Yₒₗᵢ, and the monomer, Yₘₙ, forms of flACE2-Fc-H429Y comprising two (dimerised) copies of the ACE2-Fc fusion protein (FIG. 5B) (endpoints of 10.0 and 20.9 nM respectively; FIG. 8) showed greater potency than the flACE2-Fc WT (124 nM) fusion protein and was similar to the viral neutralisation achieved with EflACE2-Fc WT (10.6 nM), but EflACE2-Fc-H429Yₒₗᵢ, was seemingly more potent (4 nM) which represents a neutralisation activity of the EflACE2-Fc WT (10.6 nM) that is over 200-fold greater than that of the unfused trACE2, or in the case of EflACE2-Fc-H429Y (4 nM), over 600 fold greater (FIG. 8).Thus, the Fc-H429Y mutation increased SARS-CoV-2 neutralisation in the trACE2-Fc and flACE2-Fc fusion protein formats, and also trended towards a greater level of potency when combined with the triple mutation of the ACE2 in the intrinsically higher affinity EflACE2-Fc.

The phenylalanine substitution of histidine 429 (H429F) of the ACE2-Fc fusion proteins did not enhance neutralisation.

Interaction of ACE2-Fc Fusion Proteins with FcγR

The interaction of FcγRIIa and FcγRIIIa with the ACE2-Fc fusion proteins was evaluated by flow cytometry using ACE2-Fc opsonised Ramos-S cells and dimeric recombinant soluble FcγR (Wines et al., 2016 supra). The trACE2-Fc, flACE2-Fc and EflACE2-Fc fusion proteins all bound to FcγRIIa and FcγRIIIa (FIG. 9A, 9B), however, the variant proteins comprising a mutated H429Y Fc component largely ablated binding to both of these Fc receptor types.

### FcγRIIIa Activation by ACE2-Fc Fusion Proteins

Antibody dependent cytotoxicity (ADCC) and Fc-dependent clearance of viruses are important antiviral effector mechanisms that may play a protective role during SARS-CoV-2 infection (Li D et al., bioRxiv doi: 10.1101/2020.12.31.424729, 2021; and Shafer A et al., J Exp Med 218(3):e20201993, 2021). Thus, in this experimentation, the ability of the trACE2-Fc, flACE2-Fc and EflACE2-Fc fusion proteins and variant proteins to activate FcγRIIIa was evaluated. It was found that Ramos-S cells opsonised with ACE2-Fc fusion proteins comprising the wild type (WT) Fc region all initiated FcγRIIIa activation (FIG. 10). However, it was surprising that the flACE2-Fc fusion protein induced a greater level of activation than trACE2-Fc indicating that the inclusion of the collectrin domain in the ACE2 component of the fusion protein substantially, and surprisingly, improves the potency of FcγRIIIa activation by the ACE2-Fc fusion protein. Further, it was found that the increased affinity that the EflACE2-Fc fusion protein has for the SARS-CoV-2 spike protein, did not alter the level of FcγRIIIa activation which was equivalent to that of flACE2-Fc (FIG. 10D).

Production of the trACE2-Fc fusion protein in the presence of kifunensine (van Berkel PHC et al., Biotechnol Bioeng 105(20:350-357, 2010), that is trACE2-Fc-kif, also improved a modest level of FcγRIIIa activation shown by trACE2-Fc to a level comparable to that of the flACE2-Fc and EflACE2-Fc fusion proteins (FIG. 10D) and approaching that of the therapeutic anti-CD20 mAb, rituximab, on the CD20 Ramos-S cells (FIG. 10A,10D). Kifunensine, a mannosidase inhibitor, prevents normal N-linked glycosylation, including core fucosylation and, in immunoglobulins, the lack of fucose on the heavy chain glycan at Asn297 is known to improve FcγRIIIa binding and activation (Ferrara C et al., Proc Natl Acad Sci USA 108(31):12669-12674, 2011). Thus, it was hypothesised that similar treatment of the flACE2-Fc and EflACE2-Fc fusion proteins, or amino acid residue substitution to increase affinity for FcγRIIIa (Wang et al., 2018 supra), would be likely to further improve their FcγRIII activating potency.

Modification of the trACE2-Fc, flACE2-Fc and EflACE2-Fc fusion proteins by including an H429F Fc component mutation did not affect FcγRIII activation by opsonised Ramos-S cells (FIG. 10). In contrast, the inclusion of the mutated H429Y Fc component in all ACE2-Fc fusion proteins ablated FcγRIIIa activation of cells, which was consistent with the abovementioned failure to bind to Fc receptors, especially FcγRIIIa (FIG. 9B). Thus, while enhancing virus neutralisation, the H429Y modified Fc component in the trACE2-Fc, flACE2-Fc and EflACE2-Fc fusion proteins were inactive in FcγR binding (FIG. 9) and in activating cells through FcγRIIIa (FIG. 10). The hierarchy of FcγRIIIa activation by the fusion proteins was flACE2-Fc WT=EflACE2-Fc>trACE2-Fc emphasising that the presence of the collectrin domain is a key component in optimal activation of FcγRIIIa by the ACE2-Fc fusion proteins.

### Complement Fixation and Activation, and Lysis of SARS-CoV-2 Spike Cells by ACE2 Fc Proteins

The ACE2-Fc fusion proteins comprising mutated Fc region components were examined for their capacity to fix complement components C1q and C5-C9 in an ELISA-based analysis using avidin-immobilised RBD-biotin (FIG. 11A-F), and importantly to mediate complement-dependent killing of cells expressing SARS-CoV-2 spike protein (FIG. 11G). When the RBD was limiting (FIG. 11A, 11B), the activity of the fusion proteins comprising a mutated H429F Fc component was surprisingly enhanced over the counterparts comprising a wild type Fc region. Additionally, the binding of C5b-9 (FIG. 11E, 11F) which forms the membrane attack complex on cells, was equivalent for the fusion proteins comprising a mutated H429F Fc region component. Further, despite their oligomerisation (FIGS. 6 and 7), a feature associated with superior CoV-2 neutralisation activities (FIG. 8), both the trACE2-Fc-H429Y and flACE2-Fc-H429Y fusion proteins showed little difference in C1q or C5-9 fixation over the counterparts comprising a wild type Fc region (FIG. 11A-F). Moreover, it was apparent that the trACE2-Fc fusion protein was more potent in fixing complement C1q than flACE2-Fc (cf FIG. 11C,11E with 11D, 11F); due to the presence of the collectrin dimerisation domain in flACE2-Fc possibly reducing the segmental flexibility of the fusion protein and thereby negatively affecting complement C1q binding. Notably however, this difference was much reduced by the inclusion of the H429F Fc mutation of the trACE2-Fc-H429F and flACE2-Fc-H429F fusion proteins; particularly evident in the fixation of the C5b-C9 complex (FIG. 11E, 11F).

Surprisingly, the fusion proteins comprising a mutated H429F Fc region component, were the only highly active ACE2-Fc fusion proteins in serum complement-dependent cytotoxicity (CDC) of Ramos-S cells (FIG. 11G). Despite the capacity of the trACE2-Fc WT, as well as the trACE2-Fc-kif fusion proteins and flACE2-Fc WT, to fix C1q and C5b-9 in the ELISA assay (FIG. 11A, 11C, 11E), these could not induce complement mediated cell death nor could EflACE2-Fc WT despite higher affinity for the SARS-CoV-2 spike protein. The trACE2-Fc-H429Yₘₙ fusion protein was also active in complement mediated killing albeit weakly.

Since it is well known that the activation of the complement cascade can also lead to, for example, the phagocytosis of cells, microbes or particles opsonised with C1q (or other complement fragments such as, for example C3b or C3bi; Ricklin D et al., Immunol Rev 274(1):33-58, 2016) which bind to specific cell surface receptors such as, for example, CR1 or CR3 (Vandendriessche S et al., Front Cell Dev Biol 9:624025, 2021) on phagocytic cells, it is considered that the observed enhancement of Fc-dependent complement lysis by the ACE2-Fc fusion proteins including an H429Y mutation, will also be reflected in similar enhancements in complement-dependent phagocytosis of ACE2-Fc-coated targets (e.g. virions) through the complement receptors.

### Conclusion

It has been found that immunotherapeutic proteins in the form of fusion proteins comprising an angiotensin converting enzyme 2 (ACE2) polypeptide (or a fragment thereof) fused to an Fc region component comprising an H429 mutation provides considerable potential for the treatment or prevention of coronavirus infection. Selection of, for example, a full length or truncated (i.e. fragment) of ACE2, and various modifications of the Fc component may also enable considerable “tuning” of the antiviral agent to modify the action(s) by which the antiviral effect is achieved. For example, by including an Fc component in the immunotherapeutic protein comprising an H429Y substitution, an oligomeric immunotherapeutic protein may be produced which shows increased virus neutralisation, and abrogated FcγR binding and activation. On the other hand, by including an Fc component in the immunotherapeutic protein comprising an H429F substitution, an antiviral effect including complement-dependent cytotoxicity (CDC) of spike protein-expressing cells (e.g. infected cells) may be achieved. Thus, these unique Fc mutations enable complementary approaches to tuning the function of the Fc region component that may aid in the development of ACE2-Fc fusion proteins and other fusion proteins of therapeutic significance by allowing the selection of desired functional profiles.

### Example 2 Chimeric Anti-TNP IgG1 Antibodies Comprising an H429 Mutation and Activity Analysis

### Methods and Materials

### Production of Anti-TNP Human IgG and Mutant Anti-TNP Human IgG Plasmid Constructs

Chimeric anti-TNP human IgG antibody constructs consisting of the variable heavy (V_{H}) and light (V_{L}) region sequence of the mouse anti-trinitrophenyl (anti-TNP) antibody TIB142 and the sequence from the constant heavy (CH) region from human IgG subclasses have been described previously in detail—hIgG1 (Patel D et al., J Immunol 184(11):6283-6292, 2010), hIgG2 and hIgG4 (Wines et al., 2016 supra). All chimeric antibody sequences were subcloned into the pCR3vector. Mutations for H429F, H429Q, H429E, H429S, H433A and H435A substitutions were made into the cDNA sequence encoding the Fc region component using standard molecular biology techniques.

### Expression and Production of IgG Antibodies by Expi293 Cells

The IgG antibodies were produced in Expi293 human embryonic kidney cells as described previously (Wines et al., 2016 supra). Briefly, Expi293 cells were maintained in Expi293 Expression Medium (Gibco, Waltham, MA, United States of America) for both cell growth and protein production. Cells were transfected simultaneously with the IgG heavy chain plasmid (15 μg) and light chain plasmid (15 μg) diluted in Opti-MEM I Reduced-Serum Medium (Gibco) using the Expifectamine transfection kit (Life Technologies) then cultured for four days. Culture supernatants were clarified by centrifugation and filtered through a 0.2 μm filter after which the IgGs were purified by affinity chromatography using a Hi-Trap HP Protein A column (GE Healthcare Life Sciences, Marlborough, MA, United States of America) and eluted with 0.1 M citric acid, pH 3.5, followed by neutralisation with 1M Tris-HCl, pH 9.0 and dialysation against PBS pH 7.5. Aggregates were removed by subsequent gel filtration on a Superose 6 10/300GL column (GE Healthcare Life Sciences) and monomeric IgG peak fractions collected. The antigen binding activity of all antibody preparations was tested on BSA-TNP by ELISA as described (Wines et al, 2016 supra).

Evaluation of Antigen Binding Activity by Monoclonal Anti-TNP mAbs

The anti-TNP mAbs were evaluated by ELISA for antigen binding activity by binding to TNP haptenylated BSA (TNP-BSA) as previously described in Wines et al., 2016 supra.

ELISA Immunoassay for Detection of Complement Fixation by mAbs

Ninety-six well flat-bottom MaxiSorp Nunc plates (Thermo Fisher Scientific) were coated with 20 μg/ml TNP-BSA in PBS overnight at 4 °C The following morning, plates were blocked with 0.1% (w/v) casein in PBS for 2 hours at 37 °C and then incubated with anti-TNP mAbs (concentrations between 4 and 0.125 μg/ml) for 2 hours at RT. To measure C1q fixation, the TNP:anti-TNP plates were incubated with 10 μg/ml purified human C1q (Merck Millipore) for 30 minutes at RT followed by 1/2000 dilution of rabbit anti-C1q IgG (Kurtovic L et al., BMC Med 17:45 2019) for 1 hour at RT. In experiments to measure C5b-C9 fixation, the TNP:anti-TNP plates were incubated with 10% fresh human serum for 30 minutes at RT followed by a 1/2000 dilution of rabbit anti-C5b-C9 (Merck Millipore) for 1 hour at RT, before washing and then incubation with goat anti-rabbit IgG conjugated to HRP (Merck Millipore) at 1/2000 dilution for 1 hour at RT followed by TMB substrate (Life Technologies) for 15-20 minutes at RT. Reactivity was stopped using 1M sulfuric acid and absorbance was measured at OD450 nm. Test samples and reagents were prepared in 0.1% (w/v) casein in PBS, and plates washed three times between each step using 0.05% (v/v) Tween20 in PBS. Samples were tested in duplicate and corrected for background reactivity using negative control wells from which antibodies were omitted.

### Results and Discussion

### Anti-TNP Antibody Constructs

The TNP-WT mAb used in this example comprised either of two human heavy chain isotypes; first, an IgG1 isotype polypeptide (SEQ ID NO: 32) comprising, in the order of the N-terminus to the C-terminus, the TNP-specific V_{H} domain of mouse monoclonal antibody TIB142 fused to CH1-hinge-CH2-CH3 domains of human IgG1, encoded by the cDNA (SEQ ID NO: 33) (Patel D et al., J Immunol 184: 6283-6292, 2010); and secondly, an IgG2 isotype polypeptide (SEQ ID NO: 34) comprised, in the order of N-terminus to the C-terminus, the TNP-specific V_{H} domain of mouse monoclonal antibody TIB142 fused to CH1-hinge-CH2-CH3 domains of human IgG2, encoded by the codon-optimised cDNA with the sequence shown as SEQ ID NO: 35. The TNP-specific light chain polypeptide (SEQ ID NO: 36) comprised the TNP-specific V_{L} domain of mAb TIB142 fused to the human kappa chain constant domain, encoded by the codon-optimised DNA with the sequence shown as SEQ ID NO: 37.

### Modification of the IgG Enhances Complement Fixation

When antibodies, such as mAbs, are bound to their target antigen the classical pathway of complement may be activated leading to physiological effects including target cell destruction by complement dependent lysis (CDC) or complement mediated antibody dependent phagocytosis (C′ADCP) by specialised phagocytic cells through specific receptors for complement proteins and fragments. Activation of this major amplifying effector system of innate immunity is critically dependent on, and initiated by, the binding of the hexameric complement protein C1q which is part of the C1 complex. This binding of C1q by the antibodies bound to their target antigens leads ultimately to the formation of the membrane attack complex (MAC) comprising the other complement components (C5b, C6, C7, C8, C9).

Since the H429F Fc region component mutations conferred potent CDC in the ACE2-Fc fusion proteins (see Example 1), the effects of these (FIG. 12) and several other mutations (FIG. 13) were evaluated in human IgG1 under varied conditions of antigen density or input mAb concentration. The Fc component mutations were examined in an ELISA for complement fixation in the context of an intact (chimeric) IgG1 antibody with mouse V domains recognising the TNP hapten and human IgG1 constant domains. Surprisingly, replacement of histidine at position 429 in the TNP-IgG1-H429F mAb resulted in enhanced C1q binding compared to the C1q binding of the unmodified TNP-IgG1-WT mAb (FIG. 12). Next, the TNP-specific mAbs were evaluated for generation of the membrane attack complex proteins (FIG. 12). Again, the TNP-IgG1-H429F mAb showed enhanced complement activation as seen by the increased formation of the MAC proteins (C5b, C6, C7, C9), and importantly, this is entirely consistent with its observed enhanced capacity to bind C1q conferred by the H429F mutation. Thus, modification at position 429 can unexpectedly alter the properties of antibodies. This enhanced function is surprising as amino acid 429 is buried within the CH3 domain of IgG1 and is distant from the complement C1q binding surface in CH2 of the H chain and from the binding site for the leukocyte Fc receptors (Hogarth PM and Pietersz GA. Nature Reviews Drug Discovery 11:311-331, 2012; Chenoweth et al., 2020 supra).

The H429F mutation in the IgG1 antibody enhanced both C1q (FIG. 12A) and C5b-C9 fixation (FIG. 12B). The complement enhanced activities of the mutant IgG1 antibody comprising an H429F mutation, particularly in fixing C1q, were most apparent when lower densities of TNP:BSA antigen were opsonised by the mutated IgG (FIG. 12). Thus, in the context of both the ACE2-Fc fusion proteins and in intact IgG1 antibodies, the H429F Fc component mutation enhances complement activation.

The antigen binding activity of the anti-TNP mAbs, TNP-IgG1-WT, TNP-IgG2-WT and the modified (mutant) mAb, TNP-IgG1-H429F, were evaluated by ELISA for binding to TNP haptenylated BSA (TNP-BSA) as described (Wines et al., 2016 supra). All of the mAbs showed substantially equivalent binding activity to TNP-BSA (FIG. 12C) indicating that the enhanced complement activation by the Fc modified anti-TNP antibodies was due to the Fc mutation and not to differences in antigen binding activity.

### Conclusion

The data obtained in this example showed that the IgG-H429F mutant was capable of enhanced C1q and C5b-C9 fixation to enable increased complement activation and can therefore be expected to be capable of inducing useful complement-based effector functions.

### Example 3 Production of Modified Forms of Therapeutic Antibodies Comprising an H429 Mutation and Analysis

In this example, the effects of modifying position H429 (i.e. by amino acid substitution) on the properties of antibodies was evaluated using a number of unrelated chimeric mAbs containing distinct antigen binding variable domains which bind to unrelated epitopes in a range of different target molecules on different cell types. These antibodies were “based” upon various commercially used, and in some cases, clinically significant mAbs. In particular, the experimentation was directed at determining the possible functional effects of amino acid substitution at position 429 in antibodies bearing the V domains of the anti-HER2 mAbs trastuzumab and pertuzumab; anti-CD20 mAbs bearing the V domains of rituximab or 11B8; and the V domain of the anti-CD38 mAb daratumumab.

### Methods and Materials

### Antibodies and Antibody Constructs

DNA sequences obtained by RT-PCR or synthetic DNA corresponding to the immunoglobulin variable and constant sequences were assembled by standard molecular biology techniques including ligation and Gibson assembly (NEBuilder, New England Biolabs) or as complete synthetic DNAs to encode entire immunoglobulin H and L chains. These sequences were utilised in expression vectors such as pcDNA3.1, pcDNA 3.4 (Thermo Fisher Scientific) and pCIneo (Promega Corporation).

The unmodified mAbs used in this example were formatted on human IgG heavy chains and produced with a human kappa light chain. The unmodified mAbs include the specific V_{H} and V_{L} domains of the indicated mAb and are referred to as the wild type (WT) forms; for instance, the “WT” trastuzumab antibody used in this example, comprised the wild type (WT) HER2-specific V_{H} and V_{L} domains of trastuzumab as previously described (https://go.drugbank.com/drugs/DB00072).

More particularly, the trastuzumab-WT mAb used in this example comprised the HER2-specific heavy chain polypeptide described at https://go.drugbank.com/drugs/DB00072 (the amino acid sequence of which is provided as SEQ ID NO: 12) comprising, in the order of N-terminus to C-terminus, the HER2-specific V_{H} domain of tmstuzumab fused to CH1-hinge-CH2-CH3 domains of human IgG1 and encoded by a codon-optimised DNA with the sequence shown as SEQ ID NO: 13. Similarly, the polypeptide of the anti-HER2 mAb trastuzumab light chain is as described at https://go.drugbank.com/drugs/DB00072 (SEQ ID NO: 14) and comprises the HER2-specific V_{L} domain of trastuzumab fused to a human kappa constant domain and is encoded by a codon-optimised DNA with the sequence shown as SEQ ID NO: 15.

The 11B8-WT mAb used in this example comprised a previously described CD20 specific heavy chain polypeptide (U.S. Pat. No. 8,529,902) (SEQ ID NO: 16) comprising, in the N-terminus to C-terminus order, the CD20-specific V_{H} domain of 11B8 fused to CH1-hinge-CH2-CH3 domains of human IgG1, and is encoded by a codon-optimised DNA with the sequence shown as SEQ ID NO: 17. Similarly, the polypeptide of the anti-CD20 mAb 11B8 light chain was as previously described (SEQ ID NO: 18) and comprised the CD-20 specific V_{L} domain of 11B8 fused to human kappa constant domain and is encoded by a codon-optimised DNA with the sequence shown as SEQ ID NO: 19.

The daratumumab-WT mAb used in this example comprised a previously described CD38-specific heavy chain polypeptide (https://go.drugbank.com/drugs/DB09331) (SEQ ID NO: 20) comprising, in the order N-terminus to C-terminus, the CD38-specific V_{H} domain of daratumumab mAb fused to CH1-hinge-CH2-CH3 domains of human IgG1, and is encoded by the codon-optimised DNA with the sequence shown as SEQ ID NO: 21. Similarly the polypeptide of the anti-CD38 mAb daratumumab light chain was as previously described at https://go.drugbank.com/drugs/DB09331 (SEQ ID NO: 22) comprising the CD38-specific V_{L} domain of the daratumumab mAb fused to human kappa constant domain is encoded by the codon-optimised DNA with the sequence shown as SEQ ID NO: 23.

The pertuzumab-WT mAb used in this example comprised a HER2-specific heavy chain polypeptide as previously described at https://go.drugbank.com/drugs/DB06366 (SEQ ID NO: 24) comprising, in the order of the N-terminus to the C-terminus, the HER2-specific V_{H} domain fused to CH1-hinge-CH2-CH3 domains of human IgG1, and is encoded by the codon-optimised DNA with the sequence shown as SEQ ID NO: 25. Similarly, the polypeptide of the anti-HER2 mAb trastuzumab light chain was as previously described at https://go.drugbank.com/drugs/DB06366 (SEQ ID NO: 26) comprising the HER2-specific V_{L} domain of the pertuzumab mAB fused to human kappa constant domain and is encoded by the codon-optimised DNA with the sequence shown as SEQ ID NO: 27.

The rituximab-WT mAb used in this example comprised a CD20-specific heavy chain polypeptide (as described at https://go.drugbank.com/drugs/DB00073) (SEQ ID NO: 28) comprising in the order N-terminus to C-terminus, the CD20-specific V_{H} domain of rituximab fused to CH1-hinge-CH2-CH3 domains of human IgG1, and is encoded by the codon-optimised DNA with the sequence shown as SEQ ID NO: 29. Similarly, the polypeptide of the anti-CD20 mAb rituximab light chain as previously described at https://go.drugbank.com/drugs/DB000723 (SEQ ID NO: 30) comprising the CD20-specific V_{L} domain of rituximab fused to human kappa constant domain, and is encoded by the codon-optimised DNA with the sequence shown as SEQ ID NO: 31.

### Synthesis of Unmodified and Mutated Heavy Chains

Antibody expression vectors were generated by standard methods that are known to those skilled in the art. Briefly, antibody expression vectors consisted of a synthetic polynucleotide sequence, encoding the antibody heavy chain or light chain, appropriately placed within plasmids such as, for example, pcDNA3 and pcDNA3.4 (Thermo Fisher Scientific). Expression vectors for antibodies of different specificities were produced by cleavage at restriction sites at the boundaries of the existing variable domain (V_{H} or V_{L}). A new synthetic DNA encoding the new V domain and flanked by sequences (e.g. 25 nucleotides) that were homologous to the cleaved vector were then incorporated by reaction with NEBuilder (New England Biolabs) according to the manufacturer's instructions.

Fc variants were produced by synthesis of synthetic polynucleotide sequences encoding the variant or by cleavage of the Fc encoding sequence of antibody expression plasmids with appropriate restriction enzymes and the incorporation of new mutagenic synthetic DNA by reaction using NEBuilder (New England Biolabs) according to the manufacturer's instructions.

### Expression of Antibody Constructs

Expression of the antibodies was conducted using transient transfection of Expi293F cells (Thermo Fisher Scientific). Expi293F cells were cultured in EXPI expression media (Life Technologies) and, 24 hours prior to transfection cells, were split to a concentration of 2×10⁶ viable cells/ml. On the day of transfection, 7.5×10⁷ viable log phase cells were centrifuged and resuspended in 25 ml of pre-warmed, antibiotic-free Expi293 Expression Media and maintained at 37 °C until transfection. Transfection of the cells was then performed at RT using the Life Technologies Expifectamine Transfection Kit as follows. Eighty microlitres of ExpiFectamine 293 reagent was diluted with 1.5 ml Opti-MEM-I Reduced Serum Medium (Gibco) and incubated for 5 minutes at RT. Thirty μg of DNA (15 μg of H chain DNA and 15 μg of L chain DNA) was diluted in 1.5 ml of Opti-MEM-I reduced serum medium then added to the diluted ExpiFectamine reagent, incubated for 20-30 minutes at RT and then added dropwise to the Expi293F cell suspension which was then cultured at 37 °C for 16 to 18 hours at which time 150 μL of the manufacturer's Enhancer 1 and 1.5 ml of Enhancer 2 were added and the cells cultured for a further four days at 37 °C

Cell cultures were harvested and centrifuged at 2500 rpm for 20-30 minutes, and the supernatant filtered with a 0.2 μm high flow filter (Sartorius AG, Göttingen, Germany) prior to purification. The presence of expected antibody in the supernatant was confirmed by SDS-PAGE.

Protein a Affinity Purification of mAbs

The mAbs were purified from the supernatant of the transfected Expi293F cells by Protein A affinity chromatography. Briefly, Hi-trap™ Protein A high-performance columns (GE Healthcare Life Sciences) were washed and equilibrated in binding buffer (20 mM NaH₂PO₄ pH 7.0) and the cell culture supernatant was loaded, the columns washed with binding buffer to baseline OD280 nm and bound antibody eluted with 0.1M sodium citrate tribasic dihydrate (pH 3.5) and 1 ml fractions collected and neutralised immediately with 1M Tris-HCl pH 9.0 and fractions containing the antibody, pooled.

Size Exclusion Chromatography (SEC) of mAbs

Following Protein A affinity purification, size exclusion chromatography (SEC) was used to further purify and characterise the antibodies. The Protein A purified antibodies were concentrated to an OD280 nm of 6-8 using a 30 kDa molecular weight cut-off centrifuge concentrator device (Merck-Millipore). Superose 6 10/300GL columns (GE Healthcare Life Sciences) were equilibrated in PBS pH 7.2, then the concentrated Protein A affinity purified antibody was loaded and separated at a flow rate of 0.5 ml/min in PBS and 0.5 ml fractions collected. For some mAbs, SEC was performed at pH 5.0, thus the concentrated Protein A purified mAb was dialysed overnight against buffer (100 mM sodium citrate, 100 mM NaCl, pH 5.0), then applied to a Superose 6 10/300 column pre-equilibrated in the same buffer. The dialysed antibody was applied to the Superose column SEC at a flow rate of 0.5 ml/min and 0.5 ml fractions collected from the column.

### Evaluation of Antigen Binding by Monoclonal Antibodies

Purified antibodies were tested for antigen binding prior to functional analysis. The mAbs recognising cell surface antigens were tested for antigen recognition by flow cytometry as previously described in Trist et al., J Immunol 192(2):792-803, 2014. The anti-CD20 mAbs based on rituximab or 11B8 and the anti-CD38 mAb based on daratumumab were tested for binding on Ramos lymphoma cells expressing CD20 and CD38. The anti-HER2 mAbs based on the trastuzumab and pertuzumab mAbs were tested on the HER2 expressing ovarian cancer cell line SK-OV-3. Briefly, mAbs were titrated by serial two-fold dilution in 25 μl of FACS buffer (PBS with 0.5% (w/v) BSA). Twenty-five microlitres of target cells at the concentration of 5×10⁶/ml were then added to the titrated mAb and incubated for 30 minutes on ice. Cells were then washed twice in FACS buffer, resuspended with 50 μL of anti-human IgG(Fab′)₂-Alexa 647 conjugate and incubated for 30 minutes on ice, washed twice in cold FACS buffer and then resuspended in 200 μl FACS buffer. The cells were analysed by flow cytometry using a BD FACSCanto™ II flow cytometer.

Flow Cytometric Detection of C1q Binding to Cells Opsonised with Monoclonal Antibody

C1q binding to antibody-opsonised cells was evaluated by flow cytometry. Ramos lymphoma cells or SK-OV-3 adenocarcinoma cells (5×10⁶/ml in FACS buffer) were incubated with serial dilutions of anti-CD20 rituximab-based or anti-HER2 trastuzumab-based antibodies respectively on ice for 30 minutes. Cells were washed twice in FACS buffer, resuspended in normal human serum diluted 1/3 in FACS buffer and incubated on ice for a further 30 minutes. The cells were then washed twice in cold FACS buffer and bound C1q was detected by resuspending the cells in rabbit antiserum detecting human C1q (1:500 dilution) and incubated for a further 30 minutes on ice. The cells were then washed twice in cold FACS buffer, resuspended in phycoerythrin-conjugated donkey antisera detecting rabbit antibody for 30 minutes, washed a further two times and resuspended in 200 μL of ice-cold FACS buffer, before being analysed on a BD FACSCanto™ II flow cytometer (Becton Dickinson).

### Complement Dependent Lysis of Cells

CDC was measured by flow cytometry using the Zombie Green Fixable Viability kit (BioLegend) following opsonisation of target cells with mAb. The mAb of interest was serially two-fold diluted starting at the initial concentration indicated on the figures, in 25 μl of PBS/BSA/G (PBS containing 0.5% (w/v) BSA and 1 mM glucose) or Lebowitz-15 (L-15) medium (containing 0.5% (w/v) BSA and lacking phenol red). Thus, for each WT mAb or modified mAb (i.e. H429-mutant mAb), or combinations thereof, 25 μl of target cells, at a concentration of 5×10⁶/ml in 25 μl of PBS/BSA/G, were then added to the titrated mAb and incubated for 30 minutes on ice. Cells were then washed twice in buffer and resuspended in 50 μl of complement (normal human serum (NHS) thawed and diluted 1:3 in buffer immediately before use), and incubated for 30 minutes at 37 °C Following the incubation, the cells were washed twice in cold buffer without BSA then resuspended in 50 μl Zombie Green (prepared in DMSO according to the manufacturer's instructions; BioLegend) diluted 1/500 in PBS and incubated for a further 30 mins on ice, protected from light. These cells were then washed once in buffer, resuspended in 50 μl of 2% paraformaldehyde/BSA/PBS/G and incubated for 30 minutes on ice, then washed once in PBS/BSA and re-suspended in 200 μl PBS/BSA/G. The cells were analysed by flow cytometry using a BD FACSCanto™ II flow cytometer (Becton Dickinson).

### MAb Cooperation to Enhance C1q Binding and CDC

Ramos cells, at 5×10⁵ cells/ml in 25 μl of PBS/BSA/G, were incubated with two-fold diluted rituximab-WT in the presence of 0.025 μg/ml daratumumab-WT or rituximab-H429F with two-fold diluted rituximab-H429F, in the presence of 0.025 μg/ml daratumumab-H429F mutant, or 0.5 μg/ml 11B8-H429F mutant, for 30 minutes at 37 °C in the presence of 1/3 dilution of normal human serum, washed twice with PBS, incubated with 1/500 Zombie Green for 30 minutes on ice washed again with PBS/BSA/G and fixed with 2% paraformaldehyde. The cells were washed, resuspended in PBS/BSA/G and analysed on a BD FACSCanto™ II flow cytometer (Becton Dickinson).

### Results and Discussion

### Antigen Binding by Monoclonal Antibodies

The antigen binding capacity of the purified antibodies used in this example were tested and confirmed prior to functional analysis.

The purified mAbs detecting cell surface antigens were also tested by flow cytometry for binding to either antigen positive Ramos cells (CD20⁺ CD38⁺) or SK-OV-3 cells (HER2⁺) and all of the mAbs showed readily detectable levels of antigen binding (FIG. 14). Moreover, within each group, the modified mAbs carrying a mutation in the IgG heavy chain, showed similar binding activity to that of the unmodified (WT) form (e.g. the anti-CD20 mAbs rituximab-WT and the rituximab-H429F and rituximab-H429Y mutants showed equivalent homogeneous binding to CD20 expressing Ramos cells). Similarly, the anti-CD38 daratumumab-WT mAb and its CH3-modified mutant, daratumumab-H429F, as well as the anti-CD20 11B8-WT and its CH3-modified mutants all gave binding profiles equivalent to their respective wild type forms (FIG. 14). Also, the anti-HER2 mAb trastuzumab-WT and its modified CH3-mutant form, trastuzumab-H429F and trastuzumab-H429Y, showed readily detectable and equivalent binding to SK-OV-3 adenocarcinoma cells as did pertuzumab-WT and its modified CH3 mutants (FIG. 14).

The effects of modifications at position 429 on antibody properties were further investigated using mAbs that bind to cell surface molecules. It was found, in particular, that substitution of H429 alters the physical characteristics of antibodies and selectively promotes antibody oligomerisation. For example, using mutants of the rituximab antibody; that is, rituximab-H429F mAb and rituximab-H429Y mAb, wherein the introduced tyrosine and phenylalanine amino acids are structurally similar, it was found that while the rituximab-H429F mAb and rituximab-H429Y mAb both eluted from Protein A affinity columns as a single homogeneous peak with elution characteristics like those of the unmodified rituximab-WT IgG (FIG. 15A), purification by size exclusion chromatography (SEC) at pH 7.2 (FIG. 16A) showed that the rituximab-H429Y mAb IgG exhibited a surprisingly different SEC profile (i.e. as compared to the profile for rituximab-WT and rituximab-H429F antibodies. That is, the rituximab-H429Y mAb SEC profile revealed two distinct peaks, one IgG peak (right of the vertical line in FIG. 16A) which was coincident with a single IgG peak in the SEC of rituximab-WT and rituximab-H429F mAbs, and a second peak considered to be containing preformed oligomers (IgG (oli)) of rituximab-H429Y (see FIG. 16A, left of the vertical line) and indicating an equilibrium between the formation of oligomeric and non-oligomeric forms. Whilst the presence of both oligomeric IgG and non-oligomeric IgG in the rituximab-H429Y mAb was surprising and different from the rituximab-WT IgG, it was even more surprising that this differed from what was observed with the rituximab-H429F mAb. Thus, the different biophysical properties of the rituximab-H429Y and rituximab-H429F mAbs is determined only by the presence of a hydroxyl in the tyrosine at position 429 of the rituximab-H429Y mAb.

Using SDS-PAGE, it was revealed that the oligomeric (IgGₒₗᵢ) and non-oligomeric (IgG) forms observed in the SEC IgG of rituximab-H429Y mAb behaved identically to each other and to the rituximab-WT IgG and rituximab-H429F mAb IgG. That is, despite its oligomeric nature, the rituximab-H429Y IgGₒₗᵢ migrated, under non-reducing conditions as a single ~150 kDa IgG species identical to its non-oligomeric rituximab-H429Y IgG (H₂L₂) form indicating, importantly, that the oligomerisation of the mAb, facilitated by the H429Y mutation was non-covalent in nature (FIG. 17A). Moreover, this clearly demonstrated that the rituximab-H429Y can exist at pH 7.2 as both IgG (IgG, H₂L₂) and as preformed oligomers of IgG (IgGₒₗᵢ) which arise from non-covalent association between the IgG heavy chains carrying the H429Y mutation. Further analysis under reducing conditions, by reduction in dithiotheitol (DTT), showed that these non-covalent oligomers of rituximab-H429Y as well as the non-oligomeric rituximab-H429Y IgG, resolved into the expected ~50 kDa heavy (H) chain and light ~25 kDa (L) chain species identical to the SDS-PAGE characteristics of rituximab-WT and the rituximab-H429F mutant.

Thus, the H429Y modification confers new characteristics on rituximab wherein, in solution at pH 7.2, the mAb exists as pre-formed, non-covalent oligomers of IgG which exist in equilibrium with single IgG molecules. In contrast, rituximab-H429F and rituximab-WT mAbs only exist in solution as the single IgG species. Thus, the choice of amino acid at position H429 unpredictably affects the physical properties of rituximab IgG.

It was also found that these effects of mutation at position H429 is independent of the V domains. That is, equivalent evaluation of H429 substitution in trastuzumab, a mAb unrelated to rituximab, and which detects a cell surface molecule (i.e. HER2) structurally distinct from CD20, achieved similar results (see FIGS. 15B, 16B and 17B). In particular, the elution of the trastuzumab-WT IgG from the Protein A affinity matrix with citrate buffer, pH 3.0, yielded a homogeneous single peak (FIG. 15B), as did elution of the trastuzumab-H429F and trastuzumab-H429Y mutant mAbs (the single IgG peak being coincident with the peak obtained of the unmodified trastuzumab-WT IgG mAb). Then, by size exclusion chromatography (SEC) at pH 7.2 (FIG. 16B), it was found that the unmodified trastuzumab-WT and the trastuzumab-H429F mAbs each contained, the expected, single major IgG species, while the trastuzumab-H429Y mAb, in contrast, showed two distinct peaks (FIG. 16B). The first trastuzumab-H429Y IgG peak was coincident with the single IgG peaks observed for trastuzumab-WT mAb and trastuzumab-H429F mAb (FIG. 16B, right of vertical line) and the second trastuzumab-H429Y IgG peak contained oligomeric IgG (IgGₒₗᵢ) (FIG. 16B, left of vertical line). In addition, SDS-PAGE (FIG. 17B) showed that under non-reducing conditions (i.e. without disulphide bond reduction), the IgG peak of the trastuzumab-WT and trastuzumab-H429F mAbs each migrated at the expected 150 kDa mass. And, in the case of the trastuzumab-H429Y mAb, SDS-PAGE (FIG. 17B) showed that under non-reducing conditions both the oligomeric trastuzumab-H429Y (IgGₒₗᵢ) and the non-oligomeric (IgG H₂L₂) species of trastuzumab-H429Y IgG migrated identically as a single 150 kDa species. This clearly demonstrated that the trastuzumab-H429Y can exist at pH 7.2 as both single IgG and as preformed oligomers of IgG arising from non-covalent association between the IgG heavy chains carrying the H429Y mutation.

Thus, the amino acid substitution at position 429 in the trastuzumab heavy chain conferred the same properties as those observed for the equivalent substitution in rituximab-based mAbs, and accordingly, the effects on the physical characteristics of the mutated mAb (particularly of oligomerisation) facilitated by H249Y mutation are independent of antibody specificity, molecular target, epitope and the V_{H} and V_{L} domains.

### Preformed Oligomeric and Non-Oligomeric H429Y IgG Antibodies Exhibit Enhanced CDC

While not wishing to be bound by theory, it is considered that oligomerisation, particularly hexamerisation, of mAbs provides the optimal basis for C1q binding and activation of the complement cascade that leads to complement-dependent effector responses (e.g. phagocytosis or killing of target cells by complement-dependent cytotoxicity (CDC)). Such oligomers/hexamers can either form in solution or on the target (ie “on target” oligomerisation or assembly).

The effect on antibody effector function arising from the H429Y modification of the CH3 domain of the IgG heavy chain was evaluated by complement dependent cytotoxicity assays (CDC) (see FIG. 18). By way of example, the effect of the H429 mutation on CDC potency was determined for the oligomeric and non-oligomeric forms of rituximab-H429Y using CD20 positive Ramos lymphoma cells as target cells. Thus, both the oligomeric rituximab-H429Y (IgG (oli)) and the non-oligomeric, IgG (H₂L₂) forms, separated by SEC at pH 7.2 (FIG. 18A) were evaluated for CDC potency and compared to the CDC of the unmodified rituximab-WT IgG. Ramos cells were incubated in the presence of serial dilutions of the rituximab-WT IgG or non-oligomeric rituximab-H429Y IgG (H₂L₂) (p1 fraction of FIG. 18A) or oligomeric rituximab-H429Y IgG H₂L₂ species (p2 fraction of FIG. 18A). Normal human serum (diluted 1/3) was then added as a source of complement and the proportion of Ramos cells killed by CDC at each mAb concentration was determined by flow cytometry (FIG. 18B). The non-oligomeric rituximab-H429Y IgG (H₂L₂) (the p1 fraction), demonstrated a surprising enhancement in CDC potency compared to the unmodified rituximab-WT. The non-covalent oligomeric IgG form of rituximab-H429Y (IgGₒₗᵢ) (the p2 fraction) also showed enhancement of CDC compared to rituximab-WT. Interestingly, both the oligomeric p2 form and the non-oligomeric p1 form exhibited similarly enhanced CDC potency which is consistent with the H429Y oligomers forming hexamers in solution at pH 7.2 prior to binding the target cell or in the case of non-oligomeric forms IgG (H₂L₂) forming hexamers on the target cell surface after antigen binding and thereby providing the optimal Fc configuration for C1q binding and thus enhanced complement activation as observed in the mAb-H429Y forms of IgG.

Thus, the substitution of H429 with tyrosine not only alters the physical characteristics of mAbs permitting oligomerisation in solution but also enhances complement dependent killing of target cells whether the IgG is oligomerised in solution or on the target.

The Oligomerisation of mAbs Including an H429Y Mutation is Controlled by Altering the pH

The nature of the non-covalent oligomerisation of the mAbs including an H429Y modification in the CH3 domain was investigated further by altering the pH of the buffer (environment) (FIG. 18C). In particular, the trastuzumab-H429Y mutant mAb (as purified by Protein A affinity chromatography) was dialysed either into buffer at pH 7.2 and then subjected to SEC also at pH 7.2, or dialysed into citrate buffer at pH 5.0 and then subjected to SEC at pH 5.0. At pH 7.2, both oligomeric and non-oligomeric IgG was present but lowering the pH of the buffer completely reversed the formation of IgG hexameric oligomers and only the IgG (H₂L₂) species was apparent (FIG. 18C). Accordingly, the H429Y modification of the CH3 domain facilitates pH-sensitive, non-covalent oligomerisation of mAbs and thereby the oligomerisation/hexamerisation in solution or on the target that enhances effector potency of complement. Moreover, the capacity to control oligomerisation by alteration of pH may be useful the manufacture of immunoglobulins carrying this modification.

### The H429F Mutation Enhances the Activation of the Serum Complement Cascade

The extent of the effect on C1q binding by modification of the CH3 domain at the H-chain position 429 was investigated by flow cytometry on cells treated with the unrelated mAbs recognising CD20 or HER2 and bearing the H429F mutation in the CH3 of their heavy chains. The results are shown in FIG. 19A-D. C1q binding was evaluated on CD20 Ramos cells treated with either rituximab-WT mAb or with rituximab-H429F mAb (FIG. 19A, 19B). Similarly, C1q binding was also measured on HER2 positive SK-OV-3 cells treated with trastuzumab-WT mAb or trastuzumab-H429F mAb (FIG. 19C, 19D). C1q bound detectably above background controls to Ramos cells treated with rituximab-WT mAb (FIG. 19A) and to SK-OV-3 cells treated with trastuzumab-WT (FIG. 19C). Importantly, C1q binding was enhanced to both the rituximab-H429F treated Ramos cells (FIG. 19B) and to trastuzumab-H429F treated SK-OV-3 cells (FIG. 19D) compared to the rituximab-WT and trastuzumab-WT. Thus, the substitution of H429 in the CH3 domain of the heavy chain enhanced C1q binding in these unrelated anti-CD20 and anti-HER2 mAbs. It is also therefore clear that this functional enhancement by H429 modification is independent of the antibody variable region and of the molecular target and the epitope detected. Moreover, enhanced C1q binding is consistent with enhanced antibody dependent activation of complement in the classical complement pathway which leads to the development of the MAC that leads to cell lysis via CDC.

Accordingly, since the binding of C1q component of the C1 complex initiates the classical complement cascade that leads to the lysis of cells, the effect of the enhanced C1q binding on the CDC killing potency was also determined using the rituximab-H429F mAb and compared to the killing potency of unmodified rituximab-WT (FIG. 19E). In particular, the proportion of Ramos cells killed by CDC was evaluated by flow cytometry at each of the indicated mAb concentrations (FIG. 19E). Like the surprising enhancement of C1q binding conferred by the H429F modification of the CH3 domain in the rituximab-H429F mAb (FIG. 19A compared to FIG. 19B), CDC killing potency (EC₅₀) was also dramatically improved (>10-fold) compared to CDC by the unmodified rituximab-WT (FIG. 19E). This surprising improvement in C1q binding and in CDC killing potency by the H429F mutation is consistent with oligomerisation (particularly hexamerisation) of the antibody on the target surface, and appears to provide the optimal configuration of the mAb Fc for the binding of C1q which is itself a hexamer, as exemplified by the analysis of the rituximab-H429F mAb.

The Enhanced Complement Activation of mAbs with H429 Mutation is Epitope Independent

As discussed above, it was found that an H429 substitution in the anti-CD20 rituximab mAb strongly increased its CDC activity; to evaluate whether the enhancement of antibody function can apply to a different epitope within a single molecular target, complement activation was investigated using a second anti-CD20 mAb. The results are shown in FIGS. 20-22.

The type-II anti-CD20 mAb, 11B8 exhibits naturally poor CDC activity, but importantly also detects an epitope distinct from type-I anti-CD20 mAb rituximab (Meyer S et al., Br J Haematol 180(6):808-820, 2018). The 11B8-WT mAb and a mutant thereof including H429F substitution were produced as described above and purified separately by Protein A affinity chromatography (FIG. 20). A single homogeneous IgG peak was obtained for the 11B8-WT and 11B8-H429F mAbs (FIG. 20) indicating that the modifications had not altered the purification characteristics of the modified mAbs compared to the unmodified 11B8-WT mAb. Also, size exclusion chromatography demonstrated that the 11B8-WT mAb contained a single non-oligomeric IgG (H₂L₂) species (FIG. 21A) as did the 11B8-H429F mAb. This was confirmed by SDS-PAGE analysis (FIG. 21B) that revealed the expected 150 kDa IgG species, prior to disulphide bond reduction for the wild type and mutant mAbs that resolved to the ~50 kDa heavy chain and 25 KD light chain species following reduction in DTT (FIG. 21B).

The CDC potency of 11B8 WT was also compared to that of the H429 modified antibody 11B8-H429F (FIG. 22). The 11B8-WT and 11B8-H429F were titrated in the presence of normal human serum as a source of complement and the percent killing of Ramos cells evaluated for each mAb concentration. It was found that the 11B8-WT mAb failed to induce appreciable CDC but in contrast, the Fc modified 11B8-H429F mAb mediated potent CDC (FIG. 22). Thus, the CDC potency is enhanced in two distinct and unrelated mAbs, rituximab-H429F and 11B8-H429F and (compare FIGS. 19E and 22), which indicates that improvement of CDC by modification of the H429 position can be achieved in different mAbs targeting distinct epitopes within the same target molecule. This also indicates that the enhanced CDC achieved by the CH3 modification is also independent of the variable domain of the modified antibody.

### The Enhancing Effect of H429F is Independent of Antibody, Molecular Target and Cell Type

The enhanced complement activating potency conferred by H429 substitution to other mAbs was evaluated in an antibody, daratumumab, recognising a fourth and unrelated molecular target, namely CD38 (de Weers M et al., J Immunol 2011; 186:1840-1848, 2011; Overdijk m B et al., MAbs 7:311-321, 2015), which is a cell surface molecule that is structurally unrelated to CD20 or HER2. The daratumumab-WT mAb was formatted as a human IgG1 and kappa light chain mAb as described above. A mutant of this antibody was engineered wherein the H429 residue was replaced with phenylalanine to create the daratumumab-H429F mAb. In each case, the daratumumab-based IgG mAbs showed equivalent characteristics and eluted from the Protein A affinity column as a single homogeneous peak (Figure). Further purification by SEC yielded a single IgG (H₂L₂) peak for the daratumumab-WT mAb and an equivalent peak also for the daratumumab-H429F mutant mAb (FIG. 24A). Moreover, SDS-PAGE confirmed that the SEC purified mAbs comprised the expected 150 kDa IgG species (prior to disulphide bond reduction), and the ~50 kDa heavy chain and 25 kDa light chain species following reduction in DTT (FIG. 24B).

The CDC potency of the daratumumab-H429F mAb was compared to daratumumab-WT mAb (FIG. 25). Each mAb was titrated individually by serial two-fold dilution in the presence of normal human serum as a source of complement. The percent killing of CD38 expressing Ramos cells was evaluated for each mAb concentration and is shown in FIG. 25. Both the daratumumab-WT and daratumumab-H429F mAbs achieved ~80% killing of Ramos lymphoma cells, however the daratumumab-H429F mAb exhibited greater CDC potency, ~15-fold higher EC50. The improved CDC potency of the daratumumab-H429F mAb was then investigated using CD38 positive cells KMS-12-PE myeloma cells that resist CDC (FIG. 26A). The daratumumab-WT mAb showed barely detectable CDC of KMS-12-PE cells above the background control lysis (~20%) in the presence only of complement (no mAb C′ only), even at concentrations 5-times greater (5 μg/ml) than that (1 μg/ml) required to maximally kill Ramos lymphoma cells (~80% lysis; see FIG. 25). In contrast, potent CDC of the KMS-12-PE cells was mediated by the daratumumab-H429F mAb (FIG. 26A). In a further experiment, CDC potency was evaluated on the CD38 expressing SUP-15 acute lymphoblastic leukaemia (ALL) cells. As can be seen from FIG. 26B, the SUP-15 cells also resisted CDC killing by the daratumumab-WT mAb, but were readily killed by the daratumumab-H429F mutant mAb. Thus, substitution at position 429 of the CH3 domain not only enhances CDC against certain targets but can also rescue potent CDC against lysis-resistant targets. Moreover, the enhanced CDC potency against the CD38 target (which is structurally distinct from CD20 detected by rituximab and 11B8, and HER2 detected by trastuzumab), also indicates that the improved efficacy is independent of target and of the epitope detected and thus also of the V domain.

H429 Substitution Facilitates Functional Synergy Between mAbs Directed at Distinct Epitopes

Functional synergy between mAbs and mediated by the H429 modification was investigated by determining the extent of binding of C1q in mixtures of mAbs (trastuzumab and pertuzumab) targeting separate epitopes in HER2.

The purification characteristics of the pertuzumab-WT mAb and a mutant wherein H429 had been replaced with phenylalanine (i.e. pertuzumab-H429F mAb) are shown in (FIGS. 27 and 28A). For each mAb, a single equivalent IgG peak was obtained from Protein A affinity chromatography (FIG. 27), and when further purified by size exclusion chromatography, single homogeneous non-oligomeric IgG species were observed (FIG. 28A, right of vertical line). SDS-PAGE analysis (FIG. 28B) confirmed that these peaks contained a 150 kDa IgG (H₂L₂) species that resolved into ~50 kDa heavy chains and ~25 kDa light chains after reduction (FIG. 28B).

Cooperation between the unrelated anti-HER2 mAbs, trastuzumab-WT and pertuzumab-WT, which detect distinct epitopes in HER2 was determined by evaluating the extent of C1q binding in mixtures of the mAbs which was quantitated by flow cytometry (FIG. 29A). Ovarian cancer cells, SK-OV-3, treated with a mixture of equal concentrations of the HER2 mAbs, trastuzumab-H429F or pertuzumab-H429F containing the H429F modification showed enhanced C1q binding (MFI=10,877) over that observed when the mAbs were used individually at the same concentrations (FIG. 29A) pertuzumab-H429F (MFI=798) or trastuzumab-H429F (MFI=1739).

The cooperative enhancement of C1q binding was further evaluated (FIG. 29B) by titration of the individual anti-HER2 mAbs or pairwise mixtures of the mAbs at two-fold serial dilutions from a starting concentration of 5 μg/ml for each mAb alone or in a mixture of the mAbs at a 1:1 ratio starting at 2.5 μg/ml:2.5 μg/ml of each mAb in the mixture (FIG. 29B). The trastuzumab-H429F and pertuzumab-H429F acted synergistically to further enhance complement activation particularly when the concentration of mAbs was limiting (e.g. at 1.25 μg/ml; FIG. 29B indicated by arrow). C1_{q} binding by the mixture of 1.25 μg/ml trastuzumab-H429F and 1.25 μg/ml Pertuzumab-H429F (MFI=6327) was greater than the C1q binding by the individual mAbs used alone at the same or twice the concentration (i.e. trastuzumab-H429F MFI=603 at 1.25 μg/ml, or 1101 at 2.5 μg/ml, and pertuzumab-H429F MFI=457 at 1.25 μg/ml, or 658 at 2.5 μg/ml) and greater than a mixture of the WT mAbs at the same 1:1 concentrations (e.g. 1.25 μg/ml trastuzumab-WT and 1.25 μg/ml pertuzumab-WT MFI=522) and even greater than the wild type mAbs used alone (i.e. trastuzumab-WT MFI=258 at 1.25 μg/ml, or 405 at 2.5 μg/ml) and pertuzumab-WT MFI=424 at 1.25 μg/ml, or MFI=483 at 2.5 μg/ml). This improved complement activation potency further indicates that greater functional potency is achievable by cooperative synergy in mixtures of CH3 domain-modified mAbs recognising distinct epitopes.

Modification of the CH3 Domain Enables mAbs of Distinct Specificity to Act Synergistically in CDC Independent of Target

The cooperative synergy of mAbs leading to greater functional potency as reflected in the enhanced C1q binding (FIG. 29) was investigated for enhanced CDC lysis of targets using mixtures of mAbs detecting either two distinct molecular structures (i.e. CD20 and CD38) or two distinct epitopes within the same molecular structure, CD20. The results are shown in FIG. 30.

Cooperation in CDC between antibodies detecting distinct molecular structures, and distinct epitopes, was determined using a mixture of rituximab-WT or rituximab-H429F mAbs that target CD20 in pairwise combination with daratumumab-WT or daratumumab-H429F mAbs which target the unrelated surface molecule, CD38. No cooperation in CDC was apparent when the rituximab-WT mAb was titrated in the presence of 0.25 μg/ml daratumumab-WT mAb (FIG. 30A); that is, the CDC observed at any concentration of rituximab-WT was no greater than the baseline CDC in the presence of 0.25 μg/ml daratumumab-WT alone (0 μg/ml rituximab, FIG. 30A) or at any concentration of the rituximab-WT alone (i.e. titrated in the absence of daratumumab-WT). However, in contrast to the unmodified wild type mAbs, the rituximab-H429F and daratumumab-H429F mAbs acted to enhance CDC cell killing (FIG. 30B). In particular, the cooperation between the mAbs in mediating CDC was readily observed in the rituximab-H429F concentration range of 0.5 μg/ml 0.125 μg/ml, when used with 0.025 μg/ml of the daratumumab-H429F mAb (NB. the enhancement is indicated by the up-arrows in FIG. 30B). CDC mediated by the rituximab-H429F mAb with the daratumumab-H429F mAb was considerably greater than with either of the H429F mAbs alone (rituximab-H429F or 0.025 μg/ml daratumumab; enhancement is indicated by the up-arrows in FIG. 30B).

Functional cooperativity between mAbs detecting distinct epitopes, but within the same target molecule, was investigated in mixtures of the different CD20 mAbs. The results are shown in FIG. 30C. Titration of the rituximab-H429F mAb in the presence (0.5 μg/ml) or absence of 11B8-H429F mAb revealed that the mAbs acted synergistically. The cooperation between the mAbs was readily detected when the concentration of both mAbs was limiting (0.25 μg/ml 0.031p g/ml rituximab-H429F and 0.05 μg/ml 11B8-H429F), where CDC was greater than with either mAb alone (enhancement indicated by the up-arrows in FIG. 30C).

Thus, mAb cooperation and synergy by modification of the antibody H chain at position 429, particularly H429F, is broad in its effect. It facilitates greater functional potency through cooperation and functional synergy in mixtures of mAbs irrespective of the epitope detected by the individual mAbs (i.e. whether the epitopes are present on the same or distinct molecular targets).

### Example 4 B Lymphocyte Killing by an Anti-CD20 Therapeutic Antibody with an H429 Mutation

Monoclonal antibodies are used in the treatment of inflammatory disease, such as autoimmune disease, by targeting normal (i.e. non-malignant) cells. For example, the anti-CD20 mAb Rituximab is used for the treatment of inflammatory disease (Lee D S W et al., Nat Rev Drug Discov 20:179-199, 2021) by targeting normal B lymphocytes known to express CD20.

### Methods and Materials

Isolation of Leukocytes from Human Peripheral Blood

Peripheral blood mononuclear cells (PBMC) were isolated from anticoagulated venous blood (Vacutainer ACD-A Becton Dickinson) by centrifugation on a Ficoll gradient. Purified cells from the plasma/ficoll interface were washed in flow cytometry buffer (L-15 medium lacking phenol red and containing 0.5% BSA (L15-BSA)) and resuspended in L15-BSA to a concentration of 5×10⁶/ml.

### Complement Dependent Lysis of Cells

The CDC killing of normal peripheral blood B lymphocytes in PBMC, mediated by WT and mutated anti-CD20 mAbs was measured by flow cytometry as described above using the Zombie Green Fixable Viability kit (BioLegend) following opsonisation of cells with mAb.

CDC was performed in 96-well plates. Cells (25 μl, 5×10⁶/ml) were reacted with equal volume of mAb in L15-BSA for 30 minutes on ice, then washed (diluted in 100-200 μl of buffer and centrifuged (at 200×g for 5 minutes, 4 °C) twice in L15-BSA. The IgG-opsonised cells were then resuspended in 50 μl of human serum diluted 1/3 in L15-BSA, as a source of complement, and incubated at 37 °C for 30 minutes. The treated cells were then washed once in L15-BSA, resuspended in 50 μl anti-CD19-APC antibody (BioLegend) in L15-BSA and incubated for a further 30 minutes on ice. Following two washes in L-15 lacking BSA, cells were then resuspended in 50 μl of Zombie Green (1/500 dilution in protein-free L-15 medium or PBS according to the manufacturer's instructions) and incubated on ice for 30 minutes. The cells were washed once in L15-BSA and the cells fixed by resuspension in 2% paraformaldehyde in buffer for 30 minutes on ice and finally washed once and then resuspended in 200 μl L15-BSA for flow cytometry analysis. B lymphocytes were identified by staining for CD19 and the proportion (%) of dead B lymphocytes specifically killed by CDC was enumerated as the percentage of CD19‘ Zombie green’ cells compared to the background control of CD19‘ Zombie green’ cells in PBMC samples treated with the negative control mAbs trastuzumab-WT or trastuzumab-H429F antibodies.

### Results and Discussion

The CDC killing of normal B lymphocytes by rituximab-H429F was investigated by flow cytometry and was compared to that of the unmodified rituximab-WT (FIG. 31). The rituximab-H429F showed greater potency, killing over 85% of B cells (FIG. 31B) compared to the much lower killing by the unmodified rituximab-WT which killed only 30% of the peripheral blood B lymphocytes (FIG. 31A). As HER2 is not expressed on B lymphocytes there was no CDC of the B lymphocytes (FIG. 31C, D) by the negative control anti-HER2 mAbs, trastuzumab-WT or trastuzumab-H429F.

### Example 5 H429F Modification Improves the Function of Immunoglobulins of Other Types

Histidine 429 is conserved in the equivalent position in all human Ig classes and subclasses (FIG. 3, FIG. 4); that is, in the CH3 domain of all IgG subclasses and IgA subclasses (FIG. 3), in IgD (FIG. 4), and also in IgE, and IgM where the CH4 domain is the equivalent of the CH3 domain of IgG (FIG. 4). The effect of modification of H429 on the function of other immunoglobulins was evaluated using human IgG3 and human IgG4 as examples.

### Methods and Materials

### Antibodies and Antibody Constructs

The mAbs used in this example comprised heavy chains of the IgG3 or IgG4 subclasses as shown in Table 5. The IgG3 heavy chain used herein contains three amino acid substitutions, N392K, M397V and R435H, introduced to avoid aggregation of purified antibodies (Saito S et al., Prot Sci 28(5):doi:10.1002/pro, 2019).

The rituximab-IgG3 CD20-specific heavy chain polypeptide (SEQ ID NO: 38) comprised in the order of the N-terminus to the C-terminus, the CD20-specific VH domain of rituximab (https://go.drugbank.com/drugs/DB00073) fused to CH1-hinge-CH2-CH3 domains of an aggregation resistant human IgG3, and is encoded by the codon-optimised DNA with the sequence shown as SEQ ID NO: 39. Similarly, the polypeptide of the anti-CD20 mAb rituximab light chain as previously described (SEQ ID NO: 30) comprising the CD20-specific VL domain of rituximab fused to human kappa constant domain (https://go.drugbank.com/drugs/DB00073), and is encoded by the codon-optimised DNA with the sequence shown as SEQ ID NO:31.

The rituximab-IgG4-WT formatted CD20-specific heavy chain polypeptide (SEQ ID NO: 40) comprised in the order of the N-terminus to the C-terminus, the CD20-specific VH domain of rituximab fused to CH1-hinge-CH2-CH3 domains of human IgG4, and is encoded by the codon-optimised DNA with the sequence shown as SEQ ID NO: 41. Similarly, the polypeptide of the anti-CD20 mAb rituximab light chain as previously described by reference (SEQ ID NO: 30) comprising the CD20-specific VL domain of rituximab fused to human kappa constant domain and is encoded by the codon-optimised DNA with the sequence shown as SEQ ID NO:31.

Modification to the mAb heavy chains were produced by synthesis of an entire antibody chain codon-optimised polynucleotide sequence including the specific modification to encode for, for example, H429F (Table 5) (GeneArt; Thermo Fisher Scientific), and antibody expression vectors were generated by standard methods known to those skilled in the art. Briefly, antibody expression vectors consisted of the synthetic polynucleotide sequence, encoding the unmodified antibody heavy chain or with the modification/mutation or light chain, appropriately placed within an expression plasmid such as, for example, pcDNA3.4 (Thermo Fisher Scientific).

### Expression of Antibody Constructs

Expression of the antibodies was conducted using transient transfection of Expi293F cells (Thermo Fisher Scientific) under the same conditions as described for rituximab and the therapeutic mAbs described elsewhere in this specification. As for all mAbs, the cell cultures were harvested after transfection and centrifuged at 2500 rpm for 20-30 minutes, and the supernatant filtered with a 0.2 μm high flow filter (Sartorius AG) prior to purification. The presence of the expected antibody in the supernatant was confirmed by SDS-PAGE electrophoresis by the method of Laemmli.

### Protein A Affinity Purification of Mab

The mAbs were purified from supernatant of the transfected Expi293F cells by Protein A affinity chromatography as described herein.

Size Exclusion Chromatography (SEC) Purification of mAbs

Following Protein A affinity purification, size exclusion chromatography (SEC) was used to further purify and characterise the antibodies as described herein.

Evaluation of Antigen Binding by Monoclonal Antibodies (mAbs)

Purified antibodies were tested for antigen binding on CD20 positive Ramos lymphoma cells by flow cytometry prior to functional analysis.

### Complement Dependent Lysis of Cells

CDC mediated by the mAbs was measured by flow cytometry as described herein using the Zombie Green Fixable Viability kit (BioLegend) following opsonisation of Ramos cells with mAb.

### Results and Discussion

The anti-CD20 mAb rituximab was formatted with unmodified aggregation-resistant IgG3 heavy chains (rituximab-IgG3), aggregation-resistant IgG3 heavy chain comprising the H429F mutation (rituximab-IgG3-H429F), wild type IgG4 heavy chains (rituximab-IgG4-WT) or IgG4 heavy chain comprising the H429F modification (rituximab-IgG4-H429F).

The rituximab-IgG3 mAb was formatted as an aggregation resistant human IgG3 and kappa light chain mAb and a mutant of this antibody was engineered wherein the H429 residue was replaced with phenylalanine to create the rituximab-IgG3(KVH)-H429F mAb. A Rituximab-IgG4-WT mAb was also produced and formatted as a human IgG4 and kappa light chain mAb. In each case, the rituximab IgG3 and IgG4 mAbs showed equivalent characteristics and eluted from the Protein A affinity column as a single homogeneous peak. Further purification by SEC yielded a single IgG (H₂L₂) peak for each of the mAbs. Moreover, SDS-PAGE confirmed that the SEC-purified mAbs comprised the expected intact IgG species (prior to disulphide bond reduction), and the ~50 kDa heavy chain and 25 kDa light chain species following reduction in dithiothreitol.

The CDC potency of the rituximab-IgG3-H429F was evaluated on Ramos cells by flow cytometry and compared to the potency of rituximab-IgG3-WT (FIG. 32A). Each mAb was titrated by serial two-fold dilution in the presence of normal human serum as a source of complement. The percentage killing of CD20-expressing Ramos cells was evaluated at each mAb concentration shown in FIG. 32A. The rituximab-IgG3-H429F mAb showed greater CDC potency than the rituximab-IgG3 mAb, particularly where the concentration of the mAb was limiting. The results are important as they demonstrate that the effects of H429 modification can be successfully incorporated into antibodies containing mutations located elsewhere in the heavy chain (e.g. the N392K, M397V and R435H mutations that overcome aggregation of the IgG). Further, the R435H mutation is known to enhance the in vivo half-life of IgG3, and thus the enhanced CDC by H429F mutation (FIG. 32A) demonstrates that H429 modification can be successfully incorporated into antibodies with other mutations affecting their in vivo half-life.

The CDC potency of rituximab-IgG4-H429F was also evaluated on Ramos cells and compared to the potency of rituximab-IgG4-WT (FIG. 32B). The unmodified rituximab-IgG4-WT failed to kill Ramos cells, but surprisingly, the H429 modified rituximab-IgG4-H429F mAb mediated readily detectable CDC.

Clearly, functional effects arising from H429F modification are not limited to human IgG1 and can be applied more broadly to other immunoglobulin types. This is significant as histidine 429 is conserved at the equivalent position in all human immunoglobulins (IgG, IgA, IgD, IgE and IgM; see FIG. 3, FIG. 4) and in the immunoglobulins of other mammals (e.g. primates).

### Example 6 CDC Killing by Antibodies with an H429 Mutation is Also Dependent on Complement

### CDC by the Mutated Antibodies is Dependent on Both Antibody and Complement

Ramos cells were opsonised with mAbs targeting different molecular targets, CD20 and CD38 or targeting epitopes within the same molecular target (CD20). CDC of Ramos cells using daratumumab-H429F or 11B8-H429F were evaluated in the presence of complement (mAb and human complement), in the presence of mAb but in the absence of complement (mAb only) or in the absence of mAb but in the presence of complement (complement only or C′ only) (FIG. 33). CDC lysis of the Ramos cells was only apparent when both mAb and complement were present. Cells opsonised only with mAb (i.e. in the absence of complement), showed little lysis under the conditions of the CDC assay. Similarly, non-opsonised cells showed little lysis when incubated only with complement. Thus, the lysis of the opsonised cells in the CDC assay was dependent on both antibody and complement.

### Example 7 H429 Modification Confers Enhanced CDC on Monoclonal Antibodies

Isatuximab is a monoclonal antibody that recognises the human CD38 cell surface molecule and which exhibits functional properties that are distinct from those of several other anti-CD38 mAbs such as daratumumab (Deckert J et al., Clin Cancer Res 20:4574-4583, 2014). Isatuximab inhibits the ADP-ribosyl cyclase activity of the CD38 molecule and detects an epitope distinct from daratumumab that inhibits the enzyme activity poorly (Deckert J et al., 2014 supra). The effects of H429 modification on the CDC activity of the functionally distinct isatuximab on cells that are resistant to CDC killing were evaluated.

### Methods and Materials

### Antibodies and Antibody Constructs

The isatuximab-WT mAb used in this example comprised a CD38-specific heavy chain polypeptide with the amino acid sequence shown as SEQ ID NO: 42 comprising, in the order of the N-terminus to the C-terminus, the CD38-specific VH domain of isatuximab mAb fused to CH1-hinge-CH2-CH3 domains of human IgG1, and is encoded by the codon-optimised DNA with the sequence shown as SEQ ID NO: 43. The polypeptide of the anti-CD38 mAb isatuximab light chain comprised the amino acid sequence shown as SEQ ID NO: 44 (comprising the CD38-specific VL domain of the isatuximab mAb fused to human kappa constant domain) and is encoded by the codon-optimised DNA with the sequence shown as SEQ ID NO: 45.

### Synthesis of Unmodified and Mutated Heavy Chains

Antibody expression vectors were generated by standard methods known to those skilled in the art. Briefly, antibody expression vectors consisted of a synthetic polynucleotide sequence, encoding the antibody heavy chain or light chain, appropriately placed within plasmids such as, for example, pcDNA3 and pcDNA3.4 (Thermo Fisher Scientific). Expression vectors for antibodies of different specificities were produced by cleavage at restriction sites at the boundaries of the existing variable domain (V_{H} or V_{L}). A new synthetic DNA encoding the new V domain and flanked by sequences (e.g. 25 nucleotides) that were homologous to the cleaved vector were then incorporated by reaction with NEBuilder (New England Biolabs) according to the manufacturer's instructions.

Fc variants were produced by synthesis of synthetic polynucleotide sequences encoding the variant or by cleavage of the Fc encoding sequence of antibody expression plasmids with appropriate restriction enzymes and the incorporation of new mutagenic synthetic DNA by reaction using NEBuilder (New England Biolabs) according to the manufacturer's instructions.

### Expression of Antibody Constructs

Expression of the antibodies was conducted using transient transfection of Expi293F cells (Thermo Fisher Scientific) under the same conditions as described above in relation to therapeutic mAbs described elsewhere in this specification. As for all mAbs, the cell cultures were harvested after transfection and centrifuged at 2500 rpm for 20-30 minutes, and the supernatant filtered with a 0.2 μm high flow filter (Sartorius AG) prior to purification. The presence of the expected antibody in the supernatant was confirmed by SDS-PAGE.

Purification of mAbs and Evaluation of Antigen Binding

The mAbs were purified from supernatant of the transfected Expi293F cells by Protein A affinity chromatography as described herein. Following Protein A affinity purification, size exclusion chromatography (SEC) was used to further purify and characterise the antibodies as described herein. Purified antibodies were then tested for antigen binding on CD38 positive SUP-15 acute lymphoblastic leukaemia (ALL) cells by flow cytometry prior to functional analysis.

### Complement Dependent Lysis of Cells

CDC mediated by the mAbs was measured by flow cytometry as described herein using the Zombie Green Fixable Viability kit (BioLegend) following opsonisation of SUP-15 acute lymphoblastic leukaemia (ALL) cells with mAb.

### Results and Discussion

The isatuximab-WT mAb was formatted as a human IgG1 and kappa light chain mAb as described above. A mutant of this antibody was engineered wherein the H429 residue was replaced with phenylalanine to create the isatuximab-H429F mAb. In each case, the isatuximab-based mAbs showed equivalent characteristics and eluted from the Protein A affinity column as a single homogeneous peak. Further purification by SEC also yielded a single IgG (H₂L₂) peak for each of the mAbs. Moreover, SDS-PAGE confirmed that the SEC-purified mAbs comprised the expected intact IgG species (prior to disulphide bond reduction), and the ~50 kDa heavy chain and 25 kDa light chain species following reduction in dithiothreitol.

The CDC potency of the isatuximab-H429F mAb was compared to isatuximab-WT mAb (FIG. 34). Each mAb was titrated individually by serial two-fold dilution in the presence of normal human serum as a source of complement. The percent CDC potency (% killing) was evaluated on the CD38 expressing SUP-15 acute lymphoblastic leukaemia (ALL) cells. As can be seen from FIG. 34, the SUP-15 cells resisted CDC killing by the isatuximab-WT mAb, but were readily killed by the isatuximab-H429F mutant mAb. Thus, substitution at position 429 of the CH3 domain not only enhances CDC against certain cell targets but can also confers potent CDC against targets that resist lysis. Importantly, the results reveal that the lysis of resistant targets can be achieved by H429 modification of unrelated mAbs that also detect distinct epitopes—isatuximab-H429F (FIG. 34) and daratumumab-H429F (FIG. 26). Additionally, the results also indicate that improved CDC efficacy can be conferred on mAbs that have other functional attributes such as the ability to inhibit target molecule function, in this example the ability of isatuximab to inhibit ADP-ribosyl cyclase action of CD38.

### Example 8 Production of Anti-Death Receptor Antibodies with an H429 Mutation

### Methods and Materials

The mAbs used in this example are listed in Table 3 below. The mAbs are designated as BDR5 and TDR5 and detect two distinct epitopes of the human death receptor 5 (DR5; Overdijk M B et al., Mol Cancer Ther 19:2126-2138, 2020) and were produced in the H₂L₂ format with heavy chains of the human IgG1, IgG2 or IgA2 subclass and their appropriate light chain of the human kappa class.

TABLE 3
DR5-specific mAbs detecting different epitopes

| | | Mutation at | |
|---|---|---|---|
| | Epitope | position 429 | Ig H-chain backbone |
| mAb Name | detected | in the H chain | (CH1-hinge-CH2-CH3) |
| BDR5-1WT | a | Wild type | IgG1 delK447 |
| BDR5-1HF | a | H429F | IgG1 delK447 |
| BDR5-2WT | a | Wild type | IgG2 delK447 |
| BDR5-2HF | a | H429F | IgG2 delK447 |
| TDR5-1WT | b | Wild type | IgG1 delK447 |
| TDR5-1HF | b | H429F | IgG1 delK447 |
| TDR5-2WT | b | Wild type | IgG2 delK447 |
| TDR5-2HF | b | H429F | IgG2 delK447 |
| BDR5-A2WT | a | Wild type | IgA delK447-Y465 |
| BDR5-A2HF | a | H429F | IgA delK447-Y465 |
| TDR5-A2WT | b | Wild type | IgA delK447-Y465 |
| TDR5-A2HF | b | H429F | IgA delK447-Y465 |

NOTE:
The H chain backbones are shown in FIG. 3, but for IgG are truncated at G446 (i.e. delK447) and thus lack the C-terminus lysine residue. The IgA H chains are truncated at G446 (i.e. delK447-Y465) and lack the 9 C-terminus amino acids. Numbering of IgG and IgA is based on the sequence alignment in FIG. 3 and FIG. 4.

Generation of DR5-Specific Antibody Constructs with IgG Heavy Chains

Immunoglobulin variable and constant region sequences were assembled by standard molecular biology techniques as complete synthetic DNA molecules to encode entire immunoglobulin H and L chains. These molecules were utilised in the expression vector pcDNA 3.4 (Thermo Fisher Scientific).

The unmodified wild type mAbs used in this example are based on the sequences of two distinct mAbs which specifically bind to the human DR5 molecule (see the DR5-specific mAbs described in U.S. Pat. No. 10,882,913; the entire disclosure of which is herein incorporated by reference). The mAbs were produced as monoclonal IgG antibodies in the H₂L₂ format.

The unmodified DR5-specific mAbs that were generated include the specific VH and VL domains of the mAb indicated in Table 3 and are referred to as the wild type (WT) forms; for instance, the “wild type” BDR5-1WT mAb antibody used in this example, comprised DR5-specific heavy and light chains wherein the DR5-specific heavy chain of the IgG1 subclass comprised the HC-hDR5-01-G56T sequence described previously but lacking the C-terminal lysine (see SEQ ID NO: 36 of U.S. Pat. No. 10,882,913). The DR5-specific kappa light chain comprised the polypeptide sequence of the LC-hDR5-01 described previously (see SEQ ID NO: 39 of U.S. Pat. No. 10,882,913). In other cases, the unmodified antibody was created by fusing the VH domain of the DR5-specific antibody to the wild type heavy (H) chain constant domains of a particular immunoglobulin subclass or class; for instance, unmodified H chains of the BDR5-2WT mAb comprised the DR5-specific VH domain polypeptide sequence of the HC-hDR5-01-G56T sequence described previously (i.e. the VH domain from SEQ ID NO: 36 of U.S. Pat. No. 10,882,913) fused to the constant domain of wild type IgG2 H chain lacking the C-terminal lysine. The DR5-specific kappa light chain of BDR5-2WT mAb comprised the polypeptide sequence of the LC-hDR5-01 described previously (i.e. the LC from SEQ ID NO: 39 of U.S. Pat. No. 10,882,913). More particularly, the BDR5-1WT antibody of the IgG1 subclass used in this example comprised the DR5-specific heavy chain polypeptide sequence of HC-hDR5-01-G56T described previously (i.e. from SEQ ID NO: 36 of U.S. Pat. No. 10,882,913), but lacking the lysine residue at the C-terminus of the H chain, the polypeptide sequence of which is provided herein as SEQ ID NO: 46, and was encoded by a codon-optimised DNA sequence (SEQ ID NO: 47). The DR5-specific kappa light chain of BDR5-1WT mAb was described previously as LC-hDR5-01 in SEQ ID NO: 39 of U.S. Pat. No. 10,882,913, and the polypeptide sequence herein is provided as SEQ ID NO: 48 and is encoded by a codon-optimised DNA with the nucleotide sequence shown as SEQ ID NO: 49. Separately, the DR5-specific BDR5-2WT antibody was formatted as a human IgG2 antibody subclass comprising, in the order of N-terminus to C-terminus, the DR5-specific VH domain from the HC-hDR5-01-G56T sequence described previously (i.e. from SEQ ID NO: 36 of U.S. Pat. No. 10,882,913) fused to CH1-hinge-CH2-CH3 domains of the human IgG2 subclass H chain lacking the lysine residue at the C-terminus of the H chain (from the polypeptide sequence provided herein as SEQ ID NO: 50) and encoded by a codon-optimised DNA sequence (SEQ ID NO: 51). The polypeptide of the DR5-specific kappa light chain of mAb BDR5-2WT is as described previously as LC-hDR5-01 (i.e. SEQ ID NO: 39 of U.S. Pat. No. 10,882,913) and comprises the polypeptide sequence provided herein as SEQ ID NO: 48 and is encoded by a codon-optimised DNA sequence of SEQ ID NO: 49). Additionally, DR5-specific antibodies denoted as TDR5 that detect an epitope that is distinct from that of the BDR5 mAbs were also generated, particularly TDR5 mAbs based on the VH domains of DR5-specific VH domain of the HC-hDR5-05 polypeptide sequence described previously (see SEQ ID No: 40 of U.S. Pat. No. 10,882,913) and produced with the DR5-specific kappa light chain having the polypeptide sequence as described previously as LC-hDR5-05 (see SEQ ID NO: 43 of U.S. Pat. No. 10,882,913). More particularly, the TDR5-1WT antibody of the IgG1 subclass used in this example comprised the DR5-specific heavy chain polypeptide sequence of HC-hDR5-05 described previously (i.e. from SEQ ID NO: 40 of U.S. Pat. No. 10,882,913) but lacking the lysine reside at the C-terminus of the H chain, the polypeptide sequence of which is provided herein as SEQ ID NO: 52, and was encoded by a codon-optimised DNA with the sequence provided as SEQ ID NO: 53. The polypeptide sequence of the DR5-specific kappa light chain of TDR5-1WT antibody is as described previously as LC-hDR5-05 in (i.e. SEQ ID NO: 43 of U.S. Pat. No. 10,882,913) and is provided as SEQ ID NO: 54 and is encoded by a codon-optimised DNA sequence (SEQ ID NO: 55).

Separately, a TDR5-2WT antibody was formatted as a human IgG2 antibody comprising, in the order of N-terminus to the C-terminus, the DR5-specific VH domain from the HC-hDR5-05 sequence described previously (i.e. from SEQ ID NO: 40 of U.S. Pat. No. 10,882,913), fused to the CH1-hinge-CH2-CH3 domains of the human IgG2 subclass lacking the lysine residue at the C-terminus of the H chain (see the polypeptide sequence provided as SEQ ID NO: 56) and encoded by a codon-optimised DNA sequence (SEQ ID NO: 57). The polypeptide of the DR5-specific kappa light chain of mAb TDR5-2WT is as described previously as LC-hDR5-05 in (i.e. SEQ ID NO: 43 of U.S. Pat. No. 10,882,913) and comprises the DR5-specific kappa light chain of TDR5-1WT antibody (SEQ ID NO: 54) and is encoded by a codon-optimised DNA sequence (SEQ ID NO: 55).

Generation of DR5-Specific Antibody Constructs with IgA H Chains

DNA sequences corresponding to the immunoglobulin variable and constant region sequences of the DR5 mAbs were assembled by standard methodologies well known to those skilled in the art as complete synthetic nucleotide sequences to encode the entire immunoglobulin H chain and the immunoglobulin L chain. These sequences were utilised in expression vectors such as pcDNA 3.4 (Thermo Fisher Scientific).

The unmodified mAbs used in this example were based on VH and VL chain domains of the DR5-specific mAbs described in U.S. Pat. No. 10,882,913 and were formatted on human IgA heavy chains and human kappa light chains (Table 3).

The unmodified IgA mAbs include the specific VH and VL domains of the indicated mAb (Table 3) and are referred to as the wild type (WT) forms; for instance, the “wild type” BDR5-A2WT mAb used in this example, comprised the DR5-specific VH domain polypeptide sequence from within the HC-hDR5-01-G56T sequence described previously (SEQ ID NO: 36 of U.S. Pat. No. 10,882,913) and the DR5-specific kappa light chain comprising the polypeptide sequence of LC-hDR5-01 sequence described previously (i.e. SEQ ID NO: 39 of U.S. Pat. No. 10,882,913). More particularly, the BDR5-A2WT mAb used in this example comprised the DR5-specific heavy chain polypeptide (the amino acid sequence of which is provided as SEQ ID NO: 58) comprising, in the order of the N-terminus to C-terminus, the DR5-specific VH domain of HC-hDR5-01-G56T sequence described previously (i.e. the VH portion of SEQ ID NO: 36 of U.S. Pat. No. 10,882,913) fused to CH1-hinge-CH2-CH3 domains of the human IgA2 subclass and encoded by a codon-optimised DNA (SEQ ID NO: 59). The polypeptide of the DR5-specific kappa light chain of the BDR5-A2WT mAb is as described previously as LC-hDR5-01 (i.e. SEQ ID NO: 39 of U.S. Pat. No. 10,882,913) and comprises the DR5-specific VL domain of LC-hDR5-01 fused to a human kappa constant domain light chain polypeptide sequence (SEQ ID NO: 48) and is encoded by a codon-optimised DNA sequence (SEQ ID NO: 49). Additional DR5-specific antibodies that were formatted as IgA mAbs were generated. In particular, the IgA WT mAb, TDR5-A2WT, was generated based on the VH and VL domains the DR5-specific HC-hDR5-05 sequences described in U.S. Pat. No. 10,882,913. More particularly, the TDR5-A2WT antibody used in this example comprised the DR5-specific heavy chain polypeptide (SEQ ID NO: 60) comprising, in the order of the N-terminus to C-terminus, the DR5-specific VH domain within the polypeptide sequence of HC-hDR5-05 described previously (SEQ ID NO: 40 of U.S. patent Ser. No. 10/882,913) fused to CH1-hinge-CH2-CH3 domains of the human IgGA2 subclass and encoded by a codon-optimised DNA sequence (SEQ ID NO: 61). The DR5-specific light chain polypeptide of the DR5-specific mAb TDR5-A2WT mAb comprised the polypeptide sequence of LC-hDR5-05 described previously (SEQ ID NO: 43 of U.S. Pat. No. 10,882,913) comprising the polypeptide sequence provided herein as SEQ ID NO: 54 and is encoded by a codon-optimised DNA sequence (SEQ ID NO: 55).

### Generation of DR5-Specific Antibody Constructs Encoding Mutated IgG and IgA Heavy Chains

MAbs comprising H chains carrying modification to the sequence at position 429, wherein histidine 429 was replaced with phenylalanine 429 (H429F) in the Fc portion of, for example, IgG1 to generate the BDR5-1HF mAb, were produced from synthetic nucleotide sequences encoding the variant by standard methodologies known to those skilled in the art, and the mutations verified by DNA sequence analysis. In the case of the human IgA H chain, the H436 as defined by the Bur numbering convention, is the equivalent of H429 position in human IgG1 (Eu numbering) and referred herein as H429 as defined by the sequence alignment shown in FIG. 3 and FIG. 4. The generated antibodies were as follows:
(i) The BDR5-1HF comprised H chains of the IgG1 subclass carrying the H429F modification and comprising the polypeptide sequence shown as SEQ ID NO: 62 encoded by a codon optimised DNA sequence (SEQ ID NO: 63).
(ii) The BDR5-2HF comprised H chains of the IgG2 subclass carrying the H429F modification and comprised the polypeptide sequence as shown in SEQ ID NO: 64 which was encoded by a codon optimised DNA sequence (SEQ ID NO: 65).
(iii) The TDR5-1HF comprised H chains of the IgG1 subclass carrying the H429F modification and comprised the polypeptide sequence as shown in SEQ ID NO: 66 which was encoded by a codon optimised DNA sequence (SEQ ID NO: 67).
(iv) The TDR5-2HF comprised H chains of the IgG2 subclass carrying the H429F modification and comprised the polypeptide sequence as shown in SEQ ID NO: 68 which was encoded by a codon optimised DNA sequence (SEQ ID NO: 69).
(v) The BDR5-A2HF comprised H chains of the IgA2 subclass carrying the H429F modification and comprised the polypeptide sequence as shown in SEQ ID NO: 70 which was encoded by a codon optimised DNA sequence (SEQ ID NO: 71).
(vi) The TDR5-A2 HF comprised H chains of the IgA2 subclass carrying the H429F modification and comprised the polypeptide sequence as shown in SEQ ID NO: 72 which was encoded by a codon optimised DNA sequence (SEQ ID NO: 73).

All H chain Fc variants were produced with their appropriate matched human kappa L chains as follows: The BDR5 H chain variants were produced with a human kappa L chain comprised of the polypeptide sequence of LC-hDR5-01 as described previously in (i.e. SEQ ID NO: 39 of U.S. Pat. No. 10,882,913) comprising the polypeptide sequence provided as SEQ ID NO: 48 and is encoded by a codon-optimised DNA with the sequence shown as SEQ ID NO: 49. The TDR5 H chain variants were produced with a human kappa L chain comprised of the polypeptide sequence of LC-hDR5-05 described previously in (i.e. SEQ ID NO: 43 of U.S. Pat. No. 10,882,913) comprising the polypeptide sequence of SEQ ID NO: 54 and is encoded by a codon-optimised DNA with the sequence shown as SEQ ID NO: 55.

Expression of the antibodies was conducted using transient transfection of Expi293F cells (Thermo Fisher Scientific). Expi293F cells were cultured in Expi293 Expression Media (Life Technologies) and, 24 hours prior to transfection cells, were split to a concentration of 2×10⁶ viable cells/ml. On the day of transfection, 7.5×10⁷ viable log phase cells were centrifuged and resuspended in 25 ml of pre-warmed, antibiotic-free Expi293 Expression Media and maintained at 37 °C until transfection. Transfection of the cells was then performed at RT using the Expifectamine Transfection Kit (Life Technologies) as follows. Eighty microlitres of ExpiFectamine 293 reagent was diluted with 1.5 ml Opti-MEM-I Reduced Serum Medium (Gibco) and incubated for 5 minutes at RT. Thirty μg of DNA (15 μg of H chain DNA and 15 μg of L chain DNA) was diluted in 1.5 ml of Opti-MEM-I reduced serum medium and was then added to the diluted ExpiFectamine reagent, incubated for 20-30 minutes at RT and then added dropwise to the Expi293F cell suspension which was then cultured at 37 °C for 16 to 18 hours at which time 150 μL of the manufacturer's Enhancer 1 and 1.5 ml of Enhancer 2 were added and the cells cultured for a further four days at 34 °C or 37 °C

Cell cultures were harvested and centrifuged at 2500 rpm for 20-30 minutes, and the supernatant filtered with a 0.2 μm high flow filter (Sartorius AG) prior to purification. The presence of the expected antibody in the supernatant was confirmed by SD S-PAGE.

Protein a Affinity Purification of IgG mAbs

The mAbs were purified from the supernatant of the transfected Expi293F cells by Protein A affinity chromatography. Briefly, Hi-trap™ Protein A high-performance columns (GE Healthcare Life Sciences) were washed and equilibrated in binding buffer (20 mM NaH₂PO₄, pH 7.0). The cell culture supernatant was loaded, the columns washed with binding buffer to baseline OD280 nm and bound antibody eluted with 0.1 M sodium citrate tribasic dihydrate (pH 3.5) and 1 ml fractions collected and neutralised immediately with 1 M Tris-HCl pH 9.0. Fractions containing the antibody were pooled.

Size Exclusion Chromatography (SEC) of IgG mAbs

Following Protein A affinity purification, size exclusion chromatography (SEC) was used to further purify and characterise the antibodies. The Protein A purified antibodies were concentrated to OD280 nm of 6-8 using 30 kDa molecular weight cut-off centrifuge concentrator device (Merck Millipore). Superose 6 10/300GL columns (GE Healthcare Life Sciences) were equilibrated in PBS, pH7.2, then the concentrated Protein A affinity purified antibody was loaded and separated at a flow rate of 0.5 ml per minute in PBS and 0.5 ml fractions collected. For some mAbs, SEC was performed at pH 5.0, thus the concentrated Protein A purified mAb was dialysed overnight against buffer (100 mM sodium citrate, 100 mM NaCl, pH 5.0), then applied to a Superose 6 10/300 column pre-equilibrated in the same buffer. The dialysed antibody was applied to the Superose column SEC at a flow rate of 0.5 ml per minute and 0.5 ml fractions collected from the column.

Evaluation of Antigen Binding by IgG or IgA mAbs

Purified antibodies were tested for DR5 antigen binding prior to functional analysis by flow cytometry as previously described in Trist et al., 2014 supra using a BD Biosciences Canto™ II flow cytometer (Becton Dickinson). The DR5-specific mAbs were tested for binding on, Colo205 colorectal cells, Ramos lymphoma cells and KMS-12-PE cells. Briefly, purified mAbs or culture supernatants from Expi293 cells producing the mAb, were titrated by serial two-fold dilution in 25 μl of FACS buffer (phosphate buffered saline with 0.5% (w/v) bovine serum albumin). Note, in the case of binding to KMS-12-PE cells, the mAbs were used at a single concentration. Twenty-five microlitres of target cells at the concentration of 5×10⁶/ml were then added to the mAb and incubated for 30 minutes on ice. Cells were then washed twice in FACS buffer, resuspended with 50 μl of anti-human IgG (Fc)-FITC conjugate (1/500 dilution; Chemicon Merck, Burlington, MA, United States of America) for detection of the bound IgG mAbs. After 30 minutes incubation on ice, they were washed and resuspended in 200 μl FACS buffer for analysis. The IgA mAbs were detected with 50 μl of biotin-conjugated anti-human IgA (0.5 μg/ml) (BD Pharmingen, San Diego, CA, United States of America) incubated for 30 minutes on ice, washed and resuspended in APC-conjugated streptavidin (1/500 dilution), washed twice in cold FACS buffer and then resuspended in 200 μl FACS buffer for analysis.

### Viability Assay

The capacity of antibodies to induce cell death was evaluated in a viability assay. The antibodies were tested either individually as single agents or as mixtures of two antibodies. The viability assay was performed using cell lines which were cultured routinely in culture medium consisting of RPMI 1640 medium supplemented with 10% (v/v) foetal calf serum plus 4 mM Glutamine and 100U/ml penicillin and 0.1 mg/ml streptomycin.

The cells were harvested from cell culture, washed in culture medium by centrifugation at 200×g and resuspended in culture medium. The cells were washed again and resuspended in culture medium to a concentration of 1.2×10⁶/ml. One hundred microlitres (100 μL) of cell suspension (10,000 cells) was added to wells of 96-well flat-bottom sterile plates. Purified mAbs either as individual mAbs or mAb mixtures were added in 100 μL of medium at a single concentration or alternatively were serially, two-fold diluted in culture medium over a concentration range (10 μg/ml to 0.075 μg/ml) and 100 μL of each dilution was added to the 96-well plates previously seeded with cells along with an additional 75 μL of culture medium to bring the total final volume in each well to 200 μL. For the mAb mixtures, the total concentration of mAb was the same as in the equivalent wells treated with the single antibodies. The plates were then incubated for 48-72 hours at 37 °C in 5% CO₂, and in some experiments, maximum cell death was determined by addition of 10 μl of 1% SDS in the last hour of the incubation (i.e. at the end of the incubation period). Following the incubation period, the viability of the cells was determined using a colorimetric assay using Cell Counting Kit 8 (CCK-8; Abcam, Cambridge, United Kingdom). This assay indicates cell viability by the production of a formazan dye upon bio-reduction of the tetrazolium salt WST-8 by living cells which is directly proportional to the number of living cells. The WST-8 solution was diluted 1:2 with culture medium and 40 μl added to each well of 96-well plates. The plates were incubated at 37 °C for 2-5 hours. Colour development was determined by measuring absorbance at 450 nm (Abs450) on a Fluorostar OPTIMA (BMG LABTECH, Ortenberg, Germany).

The viability assay analysis of IgA formatted DR5 mAbs was performed as for IgG except that cell culture supernatant from Expi293 cells producing the mAbs was used as the source of mAbs and was serially diluted as for the IgG mAbs. The maximum viability control values were determined from cells cultured in 200 μL of culture medium in the absence of antibody. The control for maximum death was determined from wells seeded only with cell suspension in 200 μL of culture medium but treated at the end of the incubation period with 10 μl of 1% SDS for 1 hour at 37 °C (i.e. in the last hour of the incubation period). Following the incubation period, the viability of the cells was determined using a colorimetric assay using Cell Counting Kit 8 (CCK-8; Abcam). This assay indicates cell viability by the production of a formazan dye upon bio-reduction of the tetrazolium salt WST-8 by living cells which is directly proportional to the number of living cells. The WST-8 solution was diluted 1:2 with culture medium and 40 μl added to each well of 96-well plates. The plates were incubated at 37 °C for 2-5 hours. Colour development was determined by measuring absorbance at 450 nm (Abs450) on a Fluorostar OPTIMA (BMG).

### Results

Induction of Apoptotic Cell Death by mAbs

The effect of antibody Fc modifications on the agonistic activity of monoclonal antibodies was evaluated using the induction of apoptosis by DR5-specific antibodies as an example. MAbs that recognise distinct epitopes on the human death receptor 5 (DR5, TRAILR2) cell surface molecule (Table 3) were produced and investigated for their capacity to induce cell death as determined in assays of cell survival. The mAbs, BDR5 and TDR5 are directed at two distinct epitopes on human DR5 and were formatted as H₂L₂ antibodies comprising DR5-specific antibody light chains and also comprising DR5-specific H chains that lacked the C-terminus lysine but were otherwise wild type human IgG1 or human IgG2 or human IgA subclass (Table 3) or otherwise comprising Fc modified DR5-specific human IgG1, IgG2 and IgA H chains wherein the histidine at position 429 of the CH3 domain of the H chain had been replaced with phenylalanine (H429F).

Quantitation of Cell Binding by IgG Formatted DR5-Specific mAbs

Purified mAbs detecting DR5 were tested by flow cytometry for binding to DR5 positive Colo205 colorectal cells (FIG. 35) or Ramos lymphoma cells (FIG. 36). All mAbs showed readily detectable levels of target antigen binding. Moreover, the Fc modified mAbs carrying a mutation in the IgG heavy chain showed identical binding activity to that of the wild type (WT) form. For example, the IgG1 formatted BDR5-1HF which includes the H429F modification showed identical binding to Colo205 cells as unmodified wild type IgG1 BDR5-1WT (FIG. 35A). The DR5-specific mAbs BDR5-2HF and BDR5-2WT formatted as IgG2 backbone (FIG. 35B) also showed identical binding activity to each other, thereby clearly demonstrating that modification of the Fc does not affect interaction of the mAbs with their target DR5 epitope. The observation that Fc modifications at position 429 do not affect antigen binding by the modified mAbs was confirmed by the analysis of TDR5 mAbs which detect a second epitope distinct from that detected by the BDR5 mAbs. Binding by TDR5-1WT and TDR5-1HF was identical (FIG. 35C) as was the binding by the IgG2 formatted forms TDR5-2WT and TDR5-2HF (FIG. 35D). Further analysis of mAb binding to DR5 expressed on Ramos lymphoma cells showed that the binding profile of mAbs was unaffected by the Fc mutation. The IgG1 formatted mAbs BDR5-1WT and BDR5-1HF (FIG. 36A) and the IgG2 formatted BDR5-2WT and BDR5-2HF (FIG. 36B) showed identical binding to Ramos lymphoma cells. The TDR5-1WT, TDR5-1HF as IgG1 WT or H429F H chains respectively (FIG. 36C) and TDR5-2WT, TDR5-2HF as IgG2 WT and H429F H chains respectively (FIG. 36D) also bound equivalently. Thus, binding to DR5 was unaffected by H429F mutation. This was further confirmed by binding to KMS-12-PE myeloma cells, where the binding of wild type or H429F mAb was identical for each mAb (e.g. BDR5-1WT and BDR5-1HF bound identically as did the IgG2 mAbs BDR5-2WT and BDR5-2HF; FIG. 37). This again confirms that the binding to the target antigen was unaffected by H429F mutation and WT and mutant mAbs bound similarly to colorectal cells, B lymphoma cells or myeloma cells.

Evaluation of the Cell Killing Potency of DR5-Specific mAbs Comprising IgG H Chains

The capacity of the DR5 mAbs to induce death of cells of different lineages was determined in a colorimetric assay that measured the survival of cells that had been cultured in the presence of the DR5-specific antibodies (FIG. 38, FIG. 39). The relative potency of mAbs comprising heavy chains carrying the H429F modification or comprising wild type H chains was determined.

### H429F Enhances DR5 mAb Induced Killing of Colorectal Cells

Colo205 colorectal cells were incubated in the presence of 20 μg/ml of the DR5 mAbs. The TDR5-1HF mAb which comprised IgG1 H chains containing the H429F modification was a potent inducer of death of Colo205 cells, Abs450 nm=1.37 by comparison to cell survival in the absence of antibody, Abs450 nm =2.24 (FIG. 38, no ab) and was substantially more potent than its wild type counterpart TDR5-1WT, which comprised wild type IgG1 heavy chains and which exhibited little capacity to induce cell death, Abs450 nm=1.99 (FIG. 38).

The effect of H429F modification on DR5 mAb comprising IgG2 heavy chains was also determined. The TDR5-2HF mAb (20 μg/ml) comprising an IgG2 heavy chain containing the H429F modification was a more potent inducer of death of Colo205 cells, Abs450 nm=1.32 (FIG. 38) by comparison to cell survival in the absence of antibody, Abs450 nm=2.24 (FIG. 38, no ab) and was substantially more potent than its wild type counterpart TDR5-2WT which comprises wild type IgG2 heavy chains, and which exhibited little capacity to induce cell death, Abs450 nm=1.91 (FIG. 38).

Thus, H429F modification of the IgG Fc improves the capacity of anti-death receptor mAbs to induce cell death resulting in reduced cell survival. This improved potency resulting from the H429F modification is independent of human IgG subclass since the TDR5-1HF and TDR5-2HF mAbs which comprise H chains of different IgG subclasses show equivalent improved potency compared to the wild type equivalents. (FIG. 38).

The TDR5 and BDR5 mAbs detect distinct epitopes on DR5. Thus, to further determine the effects of the H429F modification on the action of anti-death receptor mAbs, the Colo205 cells were incubated in a 1:1 mixture (10 μg/ml of each mAb for a total mAb concentration of 20 μg/ml) of the TDR5-2HF mixed with BDR5-2HF mAbs which are comprised of IgG2 subclass H chains carrying the H429F modification (FIG. 38). Whilst the TDR5-2HF mAb used individually showed clear improvement in killing potency (Abs450 nm=1.32) compared to its wild type counterpart (Abs450 nm =1.91), the mixture of TDR5-2HF with BDR5-2HF showed even greater improvement in killing and indeed exhibited near-total killing of cells, Abs450 nm=0.1 (FIG. 38).

### H429F Enhances DR5 mAb Induced Killing of Lymphoma Cells

Having established the killing potency of the DR5 mAbs on colorectal Colo205 cells, mAb killing potency was then investigated on cells of a different lineage, Ramos lymphoma cells (FIG. 39).

The TDR5-1HF mAb comprising IgG1 H chains containing the H429F modification and the TDR5-2HF mAb comprising IgG2 H chains containing the H429F modification were both potent inducers of death of Ramos cells, TDR5-1HF Abs450 nm=0.39, TDR5-2HF Abs450 nm=0.42 compared to cell survival in the absence of antibody, Abs450 nm=0.65 (no mAb) and were substantially more potent than their wild type counterparts comprised of wild type IgG1 or IgG2 H chains and which exhibited little capacity to induce cell death, TDR5-1WT Abs450 nm=0.59, TDR5-2WT Abs450 nm=0.51 (FIG. 39). Thus, as was observed in the killing of colorectal cells (FIG. 38), the H429F modification of the IgG Fc improves the capacity of anti-death receptor mAbs to induce cell death of lymphoma cells resulting in reduced cell survival indicating that enhanced killing potency is not cell type dependent. Further, the improved killing of the lymphoma cells resulting from the H429F modification is independent of human IgG subclass since the TDR5-1HF and TDR5-2HF mAbs which comprise H chains of different IgG subclasses (i.e. IgG1 or IgG2) show near equivalent improvement in potency compared to their IgG1 or IgG2 wild type equivalents (FIG. 39).

Since dual-epitope targeting by mixtures of mAbs comprised H chains carrying the H429F modification showed maximum killing of Colo205 colorectal cells compared to killing by the individual mAbs or wild type mAbs (FIG. 38), the same mixture was tested for capacity to kill lymphoma cells (FIG. 39). Accordingly, Ramos cells were incubated in a 1:1 mixture of the TDR5-2HF mixed with BDR5-2HF mAbs (10 μg/ml of each mAb for a total mAb concentration of 20 μg/ml) which are comprised of IgG2 subclass H chains carrying the H429F modification (FIG. 39). Whilst the TDR5-2HF mAb used individually showed clear improvement in killing potency (Abs450 nm=0.42) compared to its wild type counterpart (Abs450 nm=0.51), the mixture of TDR5-2HF with BDR5-2HF showed even greater improvement in killing and indeed exhibited near-total killing of cells, Abs450 nm=0.03 (FIG. 39).

### Effectiveness of Dual-Epitope Targeting: Killing by DR5 mAb Mixtures Matched for IgG Subclasses

The effect of H429 modification on killing potency of mixtures of mAbs directed to different epitopes was investigated using pairwise combination of mAbs (FIG. 40) incubated with Colo205 cells. In each pairwise mixture, the BDR5 and TDR5 mAbs were present in a 1:1 ratio and both mAbs comprised the same IgG heavy chain subclass with the H429F modification (BDR5-1HF with TDR5-1HF both IgG1 or BDR5-2HF with TDR5-2HF both IgG2) and their killing potency compared to their equivalent paired mAbs of wild type H chain (BDR5-1WT with TDR5-1WT both IgG1 or BDR5-2WT with TDR5-2WT both IgG2). The mixtures were serially two-fold titrated from 1 μg/ml total mAb (BDR5 mAb 0.5 μg/ml+TDR5 mAb 0.5 μg/ml) and the effect on survival of Colo205 cells determined after 48 hours. The mixture of the BDR5-1WT with TDR5-1WT comprised of the IgG1 wild type chains failed to induce appreciable killing of Colo205 cells. Indeed, cell survival in this mixture, even at the highest concentration, 1 μg/ml, was equivalent to survival in the absence of antibody (no ab, FIG. 40). Replacing the IgG subclass did not improve potency as the mix of IgG2 BDR5-2WT with TDR5-2WT also lacked killing potency even at 1 μg/ml. Thus, the lack of potency of the WT mAb mixtures was not influenced by the IgG subclass since both IgG1 and IgG2 mixtures were equally poor. However, mixtures comprised of mAbs with heavy chains containing the H429F modification were very potent inducers of killing. Mixtures of BDR5-1HF with TDR5-1HF comprising IgG1 H chains with the H429F modification achieved levels of killing that were equivalent to control for maximum death of cells determined by cell survival in the presence of SD S detergent (SDS, FIG. 40). Further, the mAb mixture BDR5-2HF with TDR5-2HF whose mAbs comprised IgG2 H chains with the H429F modification was similarly potent as their IgG1 based equivalent mAbs mixture, BDR5-1HF with TDR5-1HF. Thus, improved potency is dependent on H429F modification but is not dependent on the IgG subclass as these showed near equivalent improvements of killing whether formatted as a human IgG1 or human IgG2 subclass H chain.

### Effectiveness of Dual-Epitope Targeting: Killing by DR5 mAb Mixtures of Distinct Specificity and IgG Subclasses

Pairwise comparisons of mAbs detecting distinct epitopes of DR5 and comprising IgG heavy chains of different IgG subclasses were undertaken to determine if the superior killing potency of mAb mixtures is determined by the nature of the IgG subclass and/or epitope individual mAbs that are combined in the mixture. In particular, pairwise mixtures in a 1:1 ratio of the mAbs were made by mixing two mAbs at equal concentration (0.5 μg/ml+0.5 μg/ml) to yield a total antiDR5 mAb concentration of 1 μg/ml in the mixture (FIG. 41). The potency of the mixtures of the DR5 mAbs was then determined by culturing Colo205 cells with serial 2-fold titrated mAb mixtures wherein the DR5 mAbs in each mixture comprised different human IgG H chain subclasses (FIG. 41).

The mixture of the BDR5-1WT mAb, which comprised wild type IgG1 heavy chains, with the TDR5-2WT mAb, which comprised wild type IgG2 heavy chains (BDR5-1WT+TDR5-2WT) showed only limited killing of Colo205 cells and only at the highest concentration (0.5 μg/ml of each mAb; i.e. 1 μg/ml total mAb). In contrast, the combination of the BDR5-1HF mAb, which comprised IgG1 heavy chains containing the H429F modification, mixed with the TDR5-2HF which comprised IgG2 heavy chains containing the H429F modification (BDR5-1HF+TDR5-2HF) and mediated maximum killing comparable to levels equivalent to the SDS kill control (FIG. 41) and importantly showed greater killing potency than the mixture of their wild type mAb equivalents (BDR5-1WT+TDR5-2WT). Further, the killing potency of the mixture of the H429F modified mAbs also showed more than 10-fold greater efficacy by titration than the WT equivalents.

The reciprocal paired mixture (BDR5-2HF+TDR5-1HF) where the BDR5-2HF comprised IgG2 heavy chains containing the H429F modification and was paired with TDR5-1HF that comprised IgG1, heavy chains containing the H429F modification, also showed maximum killing levels and titre similar to the BDR5-1HF+TDR5-2HF mixture. The equivalent mixture of mAbs comprising wild type H chains (BDR5-2WT+TDR5-1WT) failed to show appreciable killing of the Colo205 cells. It is also noteworthy that the levels of killing achieved by IgG comprising H chains carrying the H429F modification was the equivalent of killing by “homogeneous” mixtures of mAbs of the same H chain subclass carrying the H429F modification for example IgG2 (BDR5-2HF with TDR5-2HF) (FIG. 40, FIG. 41).

Clearly mixtures of DR5 mAbs with wild type H chains, in any combination of pairs, showed little potency but the inclusion of H429F in the H chain transformed the killing potency irrespective of epitope specificity or of IgG subclass used in the mixture. mAbs greatly increased cell killing.

Effect on Killing by Altering the Ratio of mAbs in the Mixtures

Individual mAbs containing H429F showed improved potency over their wild type counterparts when used individually (see FIG. 38, FIG. 39, FIG. 42 and FIG. 43), but greatest potency was achieved in mixtures of mAbs comprising H chains carrying the H429F modification (FIG. 40, FIG. 41). However, the mixtures used in the experiments above (FIGS. 38-41) comprised mAbs at the same concentration (i.e. a 1:1 ratio). Thus, the effect of varying the ratio of the individual mAbs in the mixtures on the viability of cells was determined.

Cell survival was determined by culture of Colo205 cells (FIG. 42) or of Ramos cells (FIG. 43) with mixtures of BDR5-1HF mAb and TDR5-1HF mAb at a final concentration of 1 μg/ml of total DR5-specific mAb as follows: 100% BDR5-1HF with 0% TDR5-1HF, 90% BDR5-1HF with 10% TDR5-1HF, 75% BDR5-1HF with 25% TDR5-1HF, 50% BDR5-1HF with 50% TDR5-1HF, 25% BDR5-1HF with 75% TDR5-1HF, 10% BDR5-1HF with 90% TDR5-1HF and 0% BDR5-1HF with 100% TDR5-1HF. Survival of Colo205 cells was unaffected or in the case of Ramos cells was little affected, in the presence of individual mAbs comprising the wild type H chains. Indeed, for Colo205 cells treatment with individual mAbs was equivalent to survival in the absence of mAbs (no ab control, FIG. 42, FIG. 43). Survival in the presence of only individual BDR5-1HF mAb, and individual TDR5-1HF mAb both of which comprised H429F modified IgG1 H chains, was modestly reduced compared to wild type. Surprisingly, however, all mixtures showed near maximum killing of Colo205 and Ramos cells, equivalent to the SDS maximum death control. Also, surprisingly, this potent killing was apparent even when one mAb was present in the mixture at as little as 10%, for example BDR5-1HF 90:10 TDR5-1HF (FIG. 42, FIG. 43). Further, the relative abundance of a mAb of one epitope specificity in the mixture did not affect potency (i.e. the BDR5-1HF:TDR5-1HF mixes were equipotent at all ratios for example, BDR5-1HF 90:10 TDR5-1HF was as potent as BDR5-1HF 10:90 TDR5-1HF; FIG. 42, FIG. 43).

Evaluation of mAbs Comprising IgA H Chains Carrying the H429 Modification

The DR5-specific mAbs used in the IgG experiments above were also produced comprising wild type IgA H chains or comprising IgA chain containing the H429F modification, and were evaluated for their capacity to induce cell death.

The DR5-specific mAbs, BDR5 and TDR5, are directed at two distinct epitopes on human DR5 and were produced according to the method above, as antibodies comprising H chains of the human IgA and, specifically, of the human IgA2 subclass as an example (Table 3). Thus, the BDR5-A2WT mAb and the TDR5-A2WT mAb comprised wild type IgA2 subclass H chains and the BDR5-A2HF mAb and the TDR5-A2HF mAb comprised IgA2 subclass H chains carrying the equivalent of the H429F modification of IgG1 (FIG. 3, Table 3); and in all cases, the H chains were paired with the specific BDR5 or TDR5 human kappa light chains. The antibodies comprising their L chains and IgA H chains were produced in Expi293 cells and the supernatant from these cultures used as a source of DR5 mAbs.

The binding of the IgA formatted DR5-specific antibodies to target cells was evaluated using indirect immunofluorescence and quantitated by flow cytometry (FIG. 44). All mAbs detected DR5 on the Colo205 cells with a level of binding that was significantly above background control levels of non-specific binding of the fluorescent conjugate to cells in the absence of antibody (no ab) or the background level of cell fluorescence (cells) (FIG. 44). Turning to the capacity of the DR5-specific mAbs (comprising the IgA H chains) to induce cell death, this was assessed by culturing Colo205 cells in the presence of serial dilutions of the mAbs (FIG. 45). The titration curves revealed that the inclusion of the H429F in the IgA heavy chain conferred potent cell death, as the mAbs comprised of an IgA heavy chain containing the H429F modification were many-fold more potent inducers of cell death than the equivalent mAbs comprising the wild type IgA heavy chain. Thus, for example, the BDR5-A2HF (EC50>1/256 dilution) and TDR5-A2HF (EC50>1/512) were potent inducers of killing compared to the IgA BDR5-A2WT (which showed little killing at any dilution) and TDR5-A2WT (which showed killing only at high concentration (low dilution) (EC50~1/6 dilution)).

Clearly, the transformation of killing potency by the inclusion of the H429F modification in immunoglobulin heavy chains is apparent in IgA heavy chains as well as the IgG heavy chains.

Cell Specificity and Killing Potency by Mixtures of H429F Modified mAbs Detecting Different Molecular Targets

As described above, mixtures of H429F modified mAbs, where each of the H429F modified mAbs detected different epitopes on the same molecular target, showed functional improvement above their separate components at the level of C1q binding (see FIG. 29), CDC killing potency (see FIGS. 30C) and DR5-induced cell death (FIGS. 38-43). Further, improved CDC was achieved by the combination of H429 modified mAbs recognising different antigens, that is different molecular targets, such as CD20 and CD38 (FIG. 30B). Here, an experiment in mAb mediated cell death was conducted to assess the potency of mixtures of H429F modified mAbs directed at different molecular targets (e.g. CD38 and DR5). The isatuximab mAbs comprising WT or H429F mutated IgG H chains was used herein are as described in Example 7. In particular, Ramos lymphoma cells (FIG. 46) which express CD38 and DR5 were treated individually with an anti-CD38 mAb isatuximab comprising WT H chains, wild type isatuximab (Isa-WT), or with anti-DR5 mAb BDR5-1 comprising WT H chains (BDR5-1WT). The treatment with these mAbs alone resulted in little change in cell viability compared to untreated cells (A450 nm=0.720). Also, treatment with a 1:1 mixture of the Isa-WT and BDR5-1WT mAbs (Isa-WT+BDR5-1WT; FIG. 46) resulted in only a small reduction in cell viability (A450 nm=0.576). A similar reduction in viability was achieved by treatment with the individual H429F-modified mAbs, Isa-HF (A450 nm=0.550) or BDR5-1HF (A450 nm=0.564) mAbs. However, in contrast, a mixture of the Isa-HF with BDR5-1HF mAbs (Isa-HF+BDR5-1HF, FIG. 46), both of which comprise H chains carrying the H429F modification, was clearly more effective (A450 nm=0.372) in reducing cell viability by the induction of cell death.

### Discussion

The results obtained in this example clearly demonstrate that the substitution of histidine 429 in the IgG heavy chain confers increased capacity of the mAbs to induce cell death in target cells expressing a target antigen. In particular, the H429F modification of the CH3 domain was found to substantially improve the killing potency of anti-death receptor mAbs compared to mAbs with wild type H chains (i.e. lacking any modification at position 429). Importantly, the improvement in killing potency was found to be independent of the epitope on the target, independent of the IgG heavy chain subclass into which the H429 modification was made, and independent of the cellular target, and notably, the H429F modification overcame the poor killing capability of WT mAbs directed at different epitopes. In addition, it was found that the improved killing by BDR5 and TDR5 mAbs comprising the H429F modification, was achieved when these were formatted as either IgG1 and IgG2 backbones and was further increased by combining the two BDR5 and TDR5 mAbs regardless of subclass format, i.e. when the two DR5 clones were formatted as H429F modified mAbs of the same IgG subclass (IgG1 or IgG2) or when one clone was IgG1 and the second clone IgG2. In sum the effect of H429 modification of the Fc was robust, being independent of the context of IgG subclass, enhancing the receptor agonism of the mAbs, individually or in combination, as measured in these examples by DR5-induced apoptosis. Collectively, these results are very significant in that they confirm the broader utility of H429 modifications to other antibodies, antibody-like molecules and antibody derivatives, as H429 is conserved in, for example, the IgG subclasses of humans, non-human primates and mice (see the sequence comparisons provided in FIG. 3 and FIG. 4). Moreover, H429 is also found in the equivalent position in the H chains of other human Ig classes (i.e. IgA, IgE and IgM) which are structurally distinct from IgG and would not be expected to mimic the Fc-related properties attributable to the IgG Fc portion. Nonetheless, using IgA as an example, the DR5-specific mAbs used in the IgG studies were produced comprising instead wild type IgA H chains or IgA H chains containing the H429F modification, and were also found to exhibit enhanced cell killing capability.

Interestingly, the improved killing by individual mAbs carrying the H429F modification was substantially further improved by culturing target cells in a mixture of mAbs binding to different epitopes of a target antigen (e.g. different epitopes of DR5). In particular, it was found that combining TDR5-2HF and BDR5-2HF antibodies achieved a more potent level of killing than the individual mAbs (containing the H429F modification) to the extent that a mixture of the mAbs resulted in almost complete killing of the cells even at ratios of 90:10. Finally, the results of this example also showed that the improved killing achieved with mAbs including an H429 modification, whether used as individual mAbs or in mixtures, was not restricted to the target cell type and indeed, the DR5-specific antibodies, whether comprised of IgG1 or IgG2 H chains modified at H429, exhibited similar levels of enhanced killing of colorectal and lymphoma cell types.

Methods that focus killing mechanisms of action more effectively to intended target cells are desirable for improved immunotherapy with mAbs. The property of H429-modified mAbs and Fc-fusion proteins in forming oligomeric platforms (e.g. for optimal CDC) provides a route to combining mAbs with differing specificities for increased cellular target specificity. Thus, using one mAb that targets a molecule that is present on a wide range of cell types, but mixed with a mAb targeting a molecule of limited cell expression can achieve specific cell killing potency. For example, DR5 and CD38 have distinct cell and tissue distributions but they are co-expressed on some cell types particularly haemopoietic cells (Zola et al., supra 2007). The results shown in FIG. 46 demonstrate this approach and demonstrate that an individual H429F modified DR5 mAb or an individual H429F modified CD38 mAb induces modest apoptosis but lymphoma killing is potentiated by their combination where their antigens are co-expressed on the target cell. This enhancement of killing by combinations of H429F modified mAbs detecting different molecular targets may result from a number of mechanisms dependent on H429F modification of the Fc such as the formation of hetero-oligomers wherein the different cell surface targets are incorporated into the signalling cluster by virtue of the interaction between the H429F modified H chains of the different mAbs, for example, containing both CD38:anti-CD38 and DR5:anti-DR5 oligomeric complexes. Also, such potentiation may reflect the integration of signals propagated by the combined engagement and cross-linking of DR5 and CD38 by these mAbs whether homo- or hetero-oligomers or by a combination of these effects. Thus, the combined use of H429F modified mAbs that detect separate molecular target molecules combines target cell specificity with killing potency. Such a combination of effects is desirable in overcoming the often-limiting factors in therapy including lack of specificity and unwanted toxicity.

### Example 9 H429 Modification Confers Enhanced CDC on Further Example of Anti-CD38 Monoclonal Antibody

Mezagitamab is a monoclonal antibody that recognises the human CD38 cell surface molecule and which exhibits functional properties that are distinct from those of several other anti-CD38 mAbs such as daratumumab (Deckert J et al., 2014 supra). The effects of H429 modification on the CDC activity of the mezagitamab on cells that are resistant to CDC killing were evaluated.

### Methods and Materials

### Antibodies and Antibody Constructs

The mezagitamab-WT mAb used in this example comprised a CD38-specific heavy chain polypeptide with the amino acid sequence shown as SEQ ID NO: 74 comprising, in the order of the N-terminus to the C-terminus, the CD38-specific VH domain of mezagitamab mAb fused to CH1-hinge-CH2-CH3 domains of human IgG1, and which is encoded by the codon-optimised polynucleotide sequence shown as SEQ ID NO: 75. The polypeptide of the anti-CD38 mAb mezagitamab light chain comprised the amino acid sequence shown as SEQ ID NO: 76 (comprising the CD38-specific VL domain of the mezagitamab mAb fused to human lambda constant domain) and which is encoded by the codon-optimised polynucleotide sequence shown as SEQ ID NO: 77.

### Synthesis of Unmodified and Mutated Heavy Chains

Antibody expression vectors and Fc variants including the replacement of H429 with phenylalanine (H429F) in the H chains of the CD38 mAbs were generated by standard methods known to those skilled in the art and as are described elsewhere in this specification in relation to therapeutic mAbs (e.g. Example 3).

### Expression of Antibody Constructs

Expression of the antibodies was conducted using transient transfection of Expi293F cells (Thermo Fisher Scientific) under the same conditions as described above in relation to other therapeutic mAbs described in this specification. As for the mAbs, the cell cultures were harvested after transfection and centrifuged at 2500 rpm for 20-30 minutes, and the supernatant filtered with a 0.2 μm high flow filter (Sartorius AG) prior to purification. The presence of the expected antibody in the supernatant was confirmed by SDS-PAGE analysis.

Purification of mAbs and Evaluation of Antigen Binding

The mezagitamab-H429F and mezagitamab-WT mAbs were purified from supernatant of the transfected Expi293F cells using Protein A affinity chromatography followed by size exclusion chromatography. The SEC purified mAbs showed the expected H₂L₂ format of monomeric IgG confirmed by SDS-PAGE analysis. Following purification, binding to CD38 positive cells was confirmed by flow cytometric analysis (data not shown).

### Complement Dependent Lysis of Cells

CDC of target cells mediated by mAbs was measured by flow cytometry as described elsewhere in this specification (e.g. Example 3) using the Zombie Green Fixable Viability kit (BioLegend) following opsonisation of cells with mAbs.

### Results and Discussion

The capacity of the mezagitamab-H429F mAb to mediate CDC killing of cells was compared to CDC killing by its wild type counterpart mezagitamab-WT mAb (FIG. 47). Each mAb was titrated individually by serial two-fold dilution in the presence of normal human serum as a source of complement. The CDC potency (% killing) was evaluated on Ramos cells expressing the CD38 antigen. As can be seen from FIG. 47, the Ramos cells resisted CDC killing by the mezagitamab-WT mAb which only showed a maximum level of killing of 27.61% at the highest concentration (5 μg/ml) but were readily killed by the mezagitamab-H429F mutant mAb which showed a maximum killing percent of 89.74% and, even at a high dilution (0.16 μg/ml), showed 29% CDC killing which was similar to the maximum killing (27.61%) achieved by the wild type form. Thus, substitution at position H429 of the CH3 domain of mezagitamab not only enhances CDC against certain cell targets but can also confer potent CDC against targets that resist lysis. Importantly, the enhanced level of killing of cells conferred by the H429F modification in mezagitamab-H429F further demonstrates that H429 modification can be effectively applied to unrelated mAbs to enhance the killing of cells, including cells that are killed poorly by unmodified WT mAbs (i.e. cells showing resistance to lysis) as was seen, for example, with daratumumab-H429F (FIG. 25, FIG. 26) and isatuximab-H429F (FIG. 34).

### Example 10 H429F Modification of an Fc Region Component can Produce Hetero-Oligomers

The H429F modification of the IgG Fc enhances complement mediated cytotoxicity (CDC). The mutations K439E and S440K of IgG are known to suppress IgG hexamer formation and to inhibit CDC function, but full activity is restored when the two mutant IgGs are used together as a mixture (Diebolder C A et al., 2014 supra). Thus, the effects of the K439E and S440K mutations on the properties of H429 modified IgG were evaluated in this example using the CD20-specific mAb, rituximab.

### Methods and Materials

### Antibodies and Antibody Constructs

Used in this example are the rituximab IgG1 WT heavy chain and the H429F mutant heavy chain as described in Example 3 above. Two additional antibodies were synthesised which contained H429F plus an additional point mutation in the H chain as described in Table 4. The first additional mAb comprised H chains containing the H429F modification plus the additional mutation of K439E (rituximab-H429F/K439E). The second additional mAb comprised H chains containing the H429F modification plus the additional mutation of S440K (rituximab-H429F/S440K).

### Synthesis of Unmodified and Mutated Heavy Chains

Antibody expression constructs and Fc variants including the replacement of H429 with phenylalanine (H429F), K439 with glutamate (K439E) and S440 with lysine (S440K) in the H chains of the rituximab mAb were generated by standard methods known to those skilled in the art.

### Expression of Antibody Constructs

Expression of the antibodies was conducted using transient transfection of Expi293F cells (Thermo Fisher Scientific) under the same conditions described above for other therapeutic mAbs. As for the mAbs, the cell cultures were harvested after transfection and centrifuged at 2500 rpm for 20-30 minutes, and then the supernatant was filtered with a 0.2 μm high flow filter (Sartorius AG) prior to purification. The presence of the expected antibody in the supernatant was confirmed by SDS-PAGE.

Purification of mAbs and Evaluation of Antigen Binding

The mAbs were purified from supernatant of the transfected Expi293F cells by protein A affinity chromatography as described elsewhere such as in Example 3. Following protein A affinity purification, size exclusion chromatography (SEC) was used to further purify and characterise the antibodies as described herein. Purified antibodies were then tested for antigen binding on CD20 positive cells by flow cytometry prior to functional analysis.

### Complement Dependent Lysis of Cells

CDC of target cells mediated by mAbs was measured by flow cytometry using the Zombie Green Fixable Viability kit (BioLegend) following opsonisation of cells with mAbs.

TABLE 4
Expression constructs encoding the heavy chains of rituximab either encoded the WT C-terminus
sequence or were modified by mutation to encode the indicated mutant C-terminus sequences.

| Fc mutation(s) | sequence type | C-terminal sequence | |
|---|---|---|---|
| WT | amino acid | H⁴²⁹E A L H N H Y T Q K S L S L S P G K⁴⁴⁷ | SEQ ID NO: 96 |
| | DNA | CACGAAGCGCTGCACAATCATTATACTCAGAAAAGCCTGAGCTTGAGCCCGGGAAAA | SEQ ID NO: 97 |
| H429F | amino acid | F⁴²⁹E A L H N H Y T Q K S L S L S P G K⁴⁴⁷ | SEQ ID NO: 98 |
| | DNA | TTCGAGGCGCTGCACAATCATTATACTCAGAAAAGCCTGAGCTTGAGCCCGGGAAAA | SEQ ID NO: 99 |
| H429F / K439E | amino acid | F⁴²⁹E A L H N H Y T Q E S L S L S P G K⁴⁴⁷ | SEQ ID NO: 100 |
| | DNA | TTCGAAGCGCTGCACAATCATTATACTCAGGAGAGCCTGAGCTTGAGCCCGGGAAAA | SEQ ID NO: 101 |
| H429F / S440K | amino acid | F⁴²⁹E A L H N H Y T Q K K L S L S P G K⁴⁴⁷ | SEQ ID NO: 102 |
| | DNA | TTCGAAGCGCTGCACAATCATTATACTCAGAAAAAGCTGAGCTTGAGCCCGGGAAAA | SEQ ID NO: 103 |

### Results and Discussion

The rituximab-WT, rituximab-H429F and additional C-terminus mutants H429F/K439E and H429F/S440K showed equivalent elution from the protein A affinity column, and further purification by SEC also yielded a single IgG peak for each of the mAbs of 150 kDa by SDS-PAGE that comprised ~50 kDa heavy chain and 25 kDa light chain species following reduction with dithiothreitol as described for similar therapeutic mAbs in Example 3.

The CDC potency of the rituximab-H429F was compared to rituximab-WT (FIG. 48A). Each mAb was titrated individually by serial two-fold dilution in the presence of normal human serum as a source of complement. The CDC potency (% killing) was evaluated on CD20 expressing Ramos cells. As shown by FIG. 48A, the Ramos cells were resistant to CDC killing by the rituximab-WT but were readily killed by the rituximab-H429F mutant. However, both the dual mutants, rituximab-H429F/K439E and rituximab-H429F/S440K, showed greatly diminished killing compared to the rituximab-H429F. Thus, substitution at positions 439 or 440 strongly inhibits the CDC enhancing effect of the H429F mutation. Others have shown the K439E and S440K mutations inhibit Fc:Fc interactions, oligomer formation (including hexamers) and subsequent CDC, by the introduction of charge repulsion (Diebolder C A et al., 2014 supra). However, fully enhanced CDC-activity equivalent to that of the rituximab-H429F is restored when the rituximab-H429F/K439E and rituximab-H429F/S440K mAbs are used together as a mixture. It should be noted that all mAbs gave near identical binding to CD20 (FIG. 48B). Therefore, the differences in the CDC potency conferred by the mutations K439E and S440K were not related to differences in binding to the target cells. While not wishing to be bound by theory, it is considered that by using these two mutants together as a mixture allows charge repulsion to be avoided by heterotypic interactions between K439E and S440K mutant Fes.

The full CDC activity of the combination of rituximab-H429F/K439E and rituximab-H429F/S440K as a mixture demonstrates that hetero-oligomers (e.g. hexamers) are formed efficiently. This can be exploited to produce hetero-oligomers from two mAbs with different specificities to target two different epitopes on the same antigen, or two entirely different antigens. Such heterotypic oligomers, including hetero-hexamers, are expected to have advantages for specificity, for example where effector function is desirable to be directed only, or selectively, against targets that express the two specificities recognised individually by each of the two mAbs. Moreover, the crosslinking of different receptors selected by the different specificities of two mAbs in hetero-oligomers, including hetero-hexamers, is also expected to be efficient. More heterogenous oligomers (that none-the-less may have highly selective direction of effector functions to targets or may be capable of multiple crosslinks of receptors and subsequent unique biologic responses) may also be achieved by using more than two antibody specificities, such as three distinct mAbs, or four distinct mAbs etc. (eg. a hetero-oligomer formed from a mixture of mAb1-IgG-H429F/K439E, mAb2-IgG-H429F/K439E and mAb3-IgG-H429F/S440K, or from a mixture of mAb1-IgG-H429F/K439E, mAb2-IgG-H429F/K439E, mAb3-IgG-H429F/S440K and mAb4-IgG-H429F/S440K). Other mutations that suppress Fc:Fc interactions and CDC or which can be used to form hetero-oligomers have been described in van den Bremer ETJ et al., 2015 supra, and may substitute for the K439E and S440K mutations used herein.

### Example 11 Discussion of H429 Mutations in the CH3 Domain, Implications and Applications

A method of altering the functional and physical properties of the heavy (H) chain of immunoglobulins (and, in turn, the molecules comprising such H chains or a part thereof comprising at least a CH3 domain) has been identified which involves modifying the amino acids in the CH3 domain. In particular, by substitution of the histidine at position 429 (Eu numbering) within the CH3 domain with different amino acids, immunotherapeutic proteins may be produced showing unexpected and distinct effects on the physical and functional properties of the molecules such as, especially, enhancement of complement activation or mAb-induced receptor signalling as exemplified by DR5 activation, by the functional consequence of protein hexamerisation. In the context of a modified antibody according to the present disclosure, and while not wishing to be bound by theory, it is considered that oligomerisation, particularly hexamerisation, of the antibodies especially on the target (on-target assembly) provides an optimal platform for binding of the hexameric C1q complex, resulting in, for example, enhanced activation of the complement cascade that leads to complement-dependent effector responses (e.g. phagocytosis or killing of target cells by complement-dependent cytotoxicity (CDC)). Moreover, oligomerisation for the purpose of enhancing cell surface signalling of mAbs bound to targets leads to enhanced signalling responses (e.g. apoptosis and cell death through cell surface molecules able to induce death pathways). This enhancement of the DR5 signalling responses is underpinned by H429F oligomerisation and thus signal amplification induced by antibody cross-linking in other systems where signalling is achieved by antibody cross-linking; for example, antibody cross-linking of activating type receptors including the Fc receptors or antigen receptor complex of T cells and B cells or inhibitory type receptors including immune checkpoints would be expected to also be enhanced when antibodies to such cell surface molecules contain the H429F modification.

### Functional Changes

Indeed, the experimentation provided herein shows that H429 substitution can enhance the killing potency of IgG mediated by the classical complement pathway, and that antibodies bearing an H429Y or especially the H429F mutation have a greater capacity to activate complement than their wild type (WT) counterparts bearing an unmodified IgG heavy chain. Further, it was found that these modifications confer a level of enhancement of activity that is so profound that, in some instances, only the antibodies carrying a mutation at H429 are actually capable of activating complement to kill cells (i.e. the cells were otherwise resistant to killing by the unmodified wild type antibody); for example, the anti-CD20 antibody 11B8-H429F was found to potently kill lymphoma cells compared to the unmodified 11B8-WT which showed little killing capacity (FIG. 22). Similarly, the anti-CD38 antibody daratumumab-H429F kills myeloma cells and acute lymphoblastic leukaemia cells whereas the unmodified daratumumab wild type mAb does not (FIG. 26).

Further, it was significant that such enhanced functional activities (i.e. conferred by the mutation in the CH3 domain) was observed indicating that the alteration of effector functions resulting from mutation of H429 is independent of the:
(i) Variable domain (since the altered effector responses conferred by the modification of H429 were apparent in all of the antibodies detecting distinct epitopes and were independently derived and therefore unrelated, and noting that the CH3 domain is physically distant from the variable domain which recognises antigen);
(ii) Epitope (since the altered effector responses conferred by the modification of H429 were apparent in all of the antibodies detecting distinct epitopes);
(iii) Cellular target (since enhanced complement activation was observed in all of the antibodies targeting lymphoma cells, myeloma cells, adenocarcinoma cells and leukaemia cells);
(iv) Molecular target (since the functional enhancement was observed in all of the antibodies which targeted different cell surface molecules (namely HER2, CD20, CD38 and SARS-CoV-2 spike which are structurally and functionally unrelated) and also in the anti-hapten antibodies); (v) Epitope location (since the functional effects were evident in antibodies targeting different epitopes within the one molecular target (e.g. the different epitopes in CD20 recognised by the rituximab and 11B8 mAbs, and the different epitopes in HER2 recognised by the pertuzumab and trastuzumab mAbs and the different epitopes in CD38 recognised by the daratumumab and isatuximab mAbs)); (vi) Immunoglobulin type (since the altered effector responses conferred by the modification of H429 were apparent in IgG3 and in IgG4);
(vi) Physical form of the modified antibodies (since the non-covalent oligomeric/hexameric IgG form bearing the H429Y mutation and the non-oligomeric IgG bearing the same mutation are equally potent in CDC); and
(vii) Nature of the target recognition structure and the format of the H chain (since the effects seen in the antibodies bearing an H429 substitution were also evident in the ACE2-Fc fusion proteins (e.g. the H429Y modification facilitated non-covalent pH-sensitive oligomerisation and H429F enhanced complement activation)).

### Physical Changes

All of the evaluated antibodies, irrespective of their antigenic target or the nature of the substitution at position H429, showed equivalent characteristics in protein A affinity purification to their unmodified wild type mAb counterpart (e.g. the rituximab-H429F mutant antibody behaved identically to the unmodified wild type rituximab mAb). However, depending on the amino acid used to replace the H429, size exclusion chromatography (SEC) revealed that the modified mAbs can exist in solution at pH 7.2 as preformed, non-covalent IgG oligomers (IgGₒₗᵢ) in equilibrium with non-oligomeric IgG species, and the relative proportions of these oligomeric and non-oligomeric IgG, which can be equally potent in CDC (e.g. the rituximab-429Y(oli) and the non-oligomeric rituximab H429Y IgG(H₂L₂) forms both mediate enhanced CDC compared to the unmodified wild type rituximab mAb) can thereby be controlled by the pH of the buffer (e.g. lowering the purification buffer to pH 5.0 resulted in the presence of only non-oligomeric IgG compared to the higher proportion of IgGₒₗᵢ at pH 7.2) providing significant flexibility and control in the manufacture of the immunotherapeutic proteins of the disclosure.

### Cooperation and Synergy

The consequences of H429 substitution in the CH3 domain are multifaceted. For example, while such modifications can significantly enhance the potency of complement activation or receptor agonism by individual mAbs compared to their wild type counterparts, it has been found that provision of mixtures of antibodies modified at H429 may additionally enhance function by the cooperation and functional synergy between the H429 modified antibodies. In particular, it is considered that additive or synergistic co-operation in the functional activity of mAb mixtures results in even greater complement activation potency than that observed with the same H429 modified antibodies used individually and, particularly, when the concentration of each of the mAbs in the mixture was limiting. This type of cooperation was observed using mAbs detecting distinct molecular targets (e.g. the rituximab-H429F mAb detecting CD20 mixed with the daratumumab-H429F mAb detecting CD38) and in mixtures of antibodies detecting distinct epitopes within the same molecular target (e.g. the rituximab-H429F mAb mixed with 11B8-H429F binding to CD20), and found to be independent of the target cell (e.g. synergistic cooperation was observed between the pertuzumab-H429F mAb and the trastuzumab-H429F mAb on adenocarcinoma cells, as well as between the rituximab-H429F mAb and the daratumumab-H429F mAb on blood cancer cells). Such cooperation was evident in the apoptotic killing potency of anti-DR5 mAbs. These data also showed the potential to specifically form hetero-oligomers wherein the different cell surface targets are incorporated into the cluster by virtue of the interaction between the H429F modified heavy chains of the different mAbs; such hetero-oligomers, depending on context, enabling enhanced CDC or agonistic signalling leading to a cellular response such as apoptosis. Thus, the use of mixtures of mAbs with H429 substitution facilitates greater functional potency through cooperation and functional synergy irrespective of the epitope detected by the individual mAbs (i.e. whether the epitopes are present on the same or distinct molecular targets) and thereby provides the opportunity to further capitalise on the enhancement of the functional potency utility achieved by H429 modification of the immunoglobulin CH3 domain, including improving the specificity of targeting of immunotherapeutic proteins of the present disclosure by leveraging the functional cooperation apparent between different antibodies comprising H429F substitution.

Combining Other Modifications with Function-Modifying H429 Substitutions

The enhancement of complement dependent effector functions by modification at the H429 position was surprising as this amino acid residue is located in the CH3 domain whereas the interaction of C1q (with IgG) occurs primarily in the CH2 domain. Further, the interaction of an antibody with FcγR also occurs in the CH2 domain (Hogarth and Pietersz, 2012 supra; Chenoweth et al., 2020 supra) as does much of the interaction with the scavenger receptor FcRn (Dall'Acqua et al., 2002 supra). Thus, because of the modular nature of the immunoglobulin H chain (FIG. 2), it is possible to combine modifications in one domain with modifications in the same or different domains that affect antibody functions whilst retaining the oligomerisation mediated by H429 modification (e.g. an H429 substitution can be readily combined with modifications elsewhere (e.g. amino acid substitutions in the hinge and/or CH2 domain that are known to enhance or ablate one or more of Fc receptor binding and/or complement binding, or which are known to enhance or ablate antibody half-life).

Thus, in a particular application, an H429 substitution may be combined with a mutation in CH2 domain known to enhance FcγR binding (e.g. for FcγRIII binding) to improve ADCC (reviewed in Chenoweth et al., 2020 supra) in an immunotherapeutic protein of the disclosure, or alternatively (or additionally) combined with a mutation in the CH2 domain known to enhance C1q binding (Lee et al., 2017 supra) to further improve complement potency. In another particular application, an H429 substitution may be employed to enhance or decrease binding to FcRn so as to modulate the in vivo half-life of an immunotherapeutic protein. For example, in some embodiments, an H429 substitution may be combined with modifications known to enhance binding to FcRn (e.g. M252Y, S254T and/or T256E; Dall'Acqua et al., 2002 supra) so as to increase the in vivo half-life of an immunotherapeutic protein of the disclosure. Notwithstanding the modular nature of antibodies, different mutations can also be made in the same domain of the immunoglobulin chain with the resulting mutant displaying properties conferred by each of the individual mutations. For example, in some embodiments, an H429 substitution may be combined with modifications known to enhance binding to FcRn (e.g. M438L and N434S; Zalevsky J et al., Nat Biotechnol 28:157-159, 2012) so as to increase the in vivo half-life of an immunotherapeutic protein of the disclosure. In other embodiments, where the Fc region component is derived from an IgG3 immunoglobulin, an H429 substitution may be combined with the abovementioned R435H modification known to enhance the in vivo half-life of IgG3 molecules, and/or the N392K and M397V mutations (Saito S et al., 2019 supra).

Further, in some circumstances, an immunotherapeutic protein according to the present disclosure may be modified in a manner wherein the propensity to oligomerise is desirably retained (thereby enhancing the avidity of antigen binding, or in the case of fusion proteins, target binding), whilst ablating one or more effector functions such as Fc receptor interaction and/or C1q binding. As such, in the case where the immunotherapeutic protein is an antibody, the antibody will have enhanced avidity but lack immune effector functions such as ADCC or ADCP, or complement for example CDC or C′ADCP or C′ADCC. Modifications that simultaneously ablate FcγR and C1q binding are well known and include, for example, L234A, L235A or removal of the heavy chain glycan normally found at Asn 297 (Eu numbering) (Wang et al., 2018 supra). Moreover, modifications that selectively ablate FcγR and C1q binding are also well known and these can be combined with an H429 substitution so that an immunotherapeutic protein such as an antibody will have enhanced avidity but lack selected immune effector functions such as ADCC or ADCP complement, (e.g. CDC or C′ADCP or or C′ADCC). For example, modifications in the CH2 domain or the lower hinge to selectively ablate effector function, may be used in combination with the H429F substitution to produce an immunotherapeutic protein which lacks FcγR or complement-based effector systems.

In some applications, where the immunotherapeutic protein comprises two immunoglobulin H chain polypeptides, one of the H chain polypeptides (i.e. the “first H chain polypeptide”) may comprise an Fc region component (comprising at least a CH3 domain) with an amino acid substitution at a position corresponding to H429, while other (the “second H chain polypeptide”) may comprise a different modification(s) within the CH3 domain.

### Unusual Feature(s) of the Histidine at Position 429 of the IgG Heavy Chain

The H429 residue is buried within the CH3 domain of the immunoglobulin Fc portion (FIG. 1) and so does not directly participate in the protein:protein interaction interface of the Fc observed in, for example, IgG molecule assemblies, such as that visualised in the crystallographic structure of anti-HIV antibody b12 (Saphire et al., 2001 supra) nor directly in any other Fc surface interfaces that may occur. Amino acid residues that form part of the binding interfaces in protein:protein interactions are understood to be clusters of solvent accessible surface residues that contribute to a complementary binding interaction surface and, accordingly, it is considered that residues buried below such interfaces do not directly contribute to a protein:protein interaction. However, in a general sense, inaccessible residues may indirectly affect interacting surfaces, but any effect of mutation at such inaccessible residues is not simply predictable and, moreover, predictive methods focus on interfacial surface residues.

### A Summary of Some Applications of an H429 Substitution (Part 1) —Immunoglobulin Molecules

In accordance with the present disclosure, the modification to CH3 domain at position 429 can be applied to, for example, a monomeric heavy chain (i.e. H₁ format; see FIG. 2 and FIG. 53A) and thus to dimeric proteins (e.g. H chain dimers in the absence of light chain (H₂ format), or in the presence of light chain H₂L₂ format of the typical IgG structure (FIG. 2)) where the H chain dimeric molecule may be homodimeric or heterodimeric (e.g. a bi-specific antibody). Such a modification at position 429 may be made to, for example, human IgG1 or to the corresponding residue in the CH3 domain of an immunoglobulin of another IgG subclass (i.e. IgG2, IgG3 or IgG4) or an IgA subclass (i.e. IgAQ1 or IgA2) or another immunoglobulin isotype such as IgD, and even at the corresponding position of the equivalent CH4 domain of IgE or IgM. Moreover, the H429 modification may be present in only one CH3 domain of the dimer. Further, the H429 modification may be applied to monomeric antibody-like molecules (FIG. 2, FIG. 53A) comprising one or more immunoglobulin H chain polypeptides comprising at least a CH3 domain (or its equivalent or part thereof derived from IgA or other Ig classes), and such antibody-like monomers may form homodimeric or heterodimeric molecules wherein at least one monomer in the dimer is modified at the position corresponding to H429 of the IgG1 CH3 domain. The H429 modification may be present in only one CH3 domain of the dimer.

### A Summary of Some Applications of an H429 Substitution (Part 2)—Fusion/Conjugate Proteins

In accordance with the present disclosure, the modification to CH3 domain (or CH4 domain) at position H429 can be applied to, for example, a monomeric fusion protein (i.e. as exemplified by the ACE2-Fc fusion proteins of Example 1). The H429 substitution may be applied to, for example, monomeric fusion proteins (FIG. 2) comprising a CH3 domain (or its equivalent or part thereof derived from IgA or other immunoglobulin classes). Such fusion protein monomers may form homodimeric or heterodimeric molecules wherein at least one monomer in the dimer is modified at the position corresponding to H429 of the CH3 domain. In some particular applications, a fusion protein according to the disclosure may comprise an H429Y substitution in only one of the CH3 domains. Like with immunoglobulins, such H429 modifications in fusion proteins can confer effects on various physical and effector properties. For example, enhanced complement activation leading to CDC was observed to be a characteristic of ACE2-Fc fusion proteins where the H429F-substituted Fc region component conferred strong complement activation and CDC. The characteristic of pH-sensitive oligomerisation of antibodies with the H429Y substitution was also found to be a characteristic of the ACE2-Fc fusions carrying the H429Y mutation. This indicates the importance of the H429 position in dictating the physical and enhanced effector function in diverse families of immunotherapeutic proteins, and which is clearly independent of the target recognised and also of the nature of the target recognition structure (i.e. whether an antibody V domain or a cell surface receptor or soluble receptor).

### Example 12 ACE-2-Fc (with H429F) in Combination with an Anti-SARS-CoV-2 Antibody

The effect of H429 modification on the potency of CDC killing in the context of infectious disease was evaluated using mAbs detecting the spike protein of SARS-CoV-2. In particular, the histidine 429 of CH3 was substituted with phenylalanine (i.e. H429F) in the H chain of the S2P6 monoclonal antibody which targets the S2 stem region of the SARS-CoV-2 spike (see, for example, Pinto D et al., Science 373, 1109-1116, 2021).

### Methods and Materials

### Generation of Antibodies and Antibody Constructs

The S2P6-wild type (WT) mAb used in this example comprised a SARS-CoV-2 spike protein-specific heavy chain polypeptide with the amino acid sequence shown as SEQ ID NO: 78 (see Table 14) comprising, in the order of the N-terminus to the C-terminus, the spike-specific VH domain of S2P6 mAb (defined in Pinto et al., 2021 supra) fused to CH1-hinge-CH2-CH3 domains of human IgG1, and is encoded by the codon-optimised polynucleotide sequence shown as SEQ ID NO: 79 (Table 14). The polypeptide of the anti-S2P6 anti-SARS-CoV-2 light chain comprised the amino acid sequence shown as SEQ ID NO: 80 (comprising the spike-specific VL domain of the S2P6 mAb fused to human kappa constant domain) and is encoded by the codon-optimised polynucleotide sequence shown as SEQ ID NO: 81 (Table 14).

Two additional mAbs were generated, the CC40.8-WT mAb and the CV3-25-WT mAb, and used in this example. Each mAb comprised a SARS-CoV-2 spike protein-specific heavy chain polypeptide with the amino acid sequence for either CC40.8 (shown as SEQ ID NO: 82) or for CV3-25 shown as (SEQ ID NO: 83) comprising, in the order of the N-terminus to the C-terminus, the spike-specific VH domain of either CC40.8 mAb (defined in Zhou P et al., Sci Transl Med 14(637):eabi9215, 2022) or of CV3-25 (defined in Jennewein M F et al., Cell Rep 36(2):109353, 2021) fused to CH1-hinge-CH2-CH3 domains of human IgG1, and encoded by the codon-optimised polynucleotide sequence shown for CC40.8 as SEQ ID NO: 84, or for CV3-25 as SEQ ID NO: 85 (see Table 14 for the sequences). The polypeptide of the anti-spike CC40.8 light chain or of the anti-spike CV3-25 light chain comprised the amino acid sequence shown in Table 14 for CC40.4 as SEQ ID NO: 86 and for CV3-25, shown as SEQ ID NO: 87 (comprising the spike-specific VL domain of the CC40.8 mAb or of CV3-25 fused to human kappa constant domain) and are encoded by the codon-optimised polynucleotide sequence shown for CC40.8 as SEQ ID NO: 88 and for CV3-25 as the codon-optimised DNA sequence shown as SEQ ID NO: 89.

### Synthesis of Unmodified and Mutated Heavy Chains

Antibody expression vectors which included a modified Fc sequence encoding the substitution of H429 with phenylalanine (i.e. H429F) in the H chains of the S2P6, CC40.8 and CV3-25 mAbs, were generated by standard methodologies known to those skilled in the art. The respective modified H chain amino acid sequences (and encoding polynucleotide sequences) are shown in Table 14 as SEQ ID NOs: 90-95.

Expression and Purification of flACE2-Fc Fusion Proteins

The flACE2-Fc-WT and flACE2-Fc-H429F fusion proteins were produced using transient transfection in Expi293F cells as described above in Example 1. Purification from supernatant by ion exchange chromatography and size exclusion chromatography (under the same conditions described in Example 1) yielded a single fusion protein “species”.

### Expression of Anti-SARS-CoV-2 Antibody Constructs

Expression of the anti-SARS-CoV-2 spike antibodies was conducted using transient transfection of Expi293F cells (Thermo Fisher Scientific) using standard methodologies. The cell cultures were harvested after transfection and centrifuged at 2500 rpm for 20-30 minutes, and the supernatant filtered with a 0.2 μm high flow filter (Sartorius AG) prior to purification. The presence of the expected antibody in the supernatant was confirmed by SD S-PAGE.

Purification of mAbs and Evaluation of Antigen Binding

The anti-SARS-CoV-2 mAbs were purified from supernatant of the transfected Expi293F cells by protein A affinity chromatography then size exclusion chromatography (SEC) yielding the expected single IgG peak (i.e. for the standard H₂L₂ antibody structure) confirmed by SDS-PAGE. Prior to functional analysis, the purified flACE2-Fc-H429F or flACE2-Fc-WT fusion proteins and the purified anti-SARS-CoV-2 mAbs were tested for antigen binding on Ramos cells expressing SARS-CoV-2 spike (Ramos-S cells) by flow cytometry using a fluorescent anti-human IgG-Fc antibody (FITC-conjugated anti-hIgG-Fc, 1/500; Chemicon®, Merck KGaA, Darmstadt, Germany).

### Complement Dependent Lysis of Cells

The potency of CDC mediated by the flACE2-Fc fusion protein or the anti-SARS-CoV-2 mAbs was measured by flow cytometry using Ramos-S cells expressing the SARS-CoV-2 spike protein in a similar manner to the CDC assay described in Example 1. Human serum, diluted 1/6.4, was used as a source of complement. The Zombie Green Fixable Viability kit (BioLegend) was used following opsonisation of Ramos-S cells with the mAbs. Additionally, CDC lysis was examined in mixtures of flACE2-Fc-H429F and anti-SARS-CoV-2 mAbs comprising H chains with an H429F modification.

### Results and Discussion

H429 Modification Confers CDC by the flACE2-Fc Fusion Protein

The flACE2-Fc-H429F and flACE2-Fc-WT fusion proteins exhibited near identical binding characteristics to SARS-CoV-2 spike protein expressed on Ramos-S cells (FIG. 49A) indicating that the H429F mutation did not appreciably affect antigen binding on cells. The flACE2-Fc-H429F and flACE2-Fc-WT fusion proteins were then tested for their capacity to mediate CDC (FIG. 49B). Despite their similar binding profiles, considerable differences in the CDC potency were observed between the two fusion proteins. That is, flACE2-Fc-WT showed poor CDC of Ramos-S cells, with the maximum kill of 19.4% occurring at the highest concentration (2.5 μg/ml flACE2-Fc-WT) and rapid titration to background (2.5 μg/ml 0.6 μg/ml). In contrast, the flACE2-Fc-H429F fusion protein mediated a potent level of cell killing over a larger concentration range (2.5 μg/ml 0.156 μg/ml) compared to flACE2-Fc-WT, and exhibited a maximum kill of 73.8%. This is consistent with the results of ELISA experiments (FIG. 11) showing that flACE2-Fc-H429F demonstrated enhanced binding of C1q (FIG. 11D) and enhanced formation of the membrane attack complex (FIG. 11F) compared to its WT counterpart as was also demonstrated for trACE2-Fc-H429F (FIG. 11C, 11E).

H429 Modification Confers CDC by Anti-SARS-CoV-2 mAbs

The anti-SARS-CoV-2 mAbs S2P6-WT, CC40.8-WT and CV-23-WT were formatted as wild type human IgG1 and kappa light chain mAbs as described above. The H429F mutation was engineered into the H chains of each mAb to generate S2P6-H429F, CC40.8-H429F and CV-23-H429F mAbs. The binding of the purified mAbs to SARS-CoV-2 spike protein was evaluated by flow cytometry (FIG. 49C). The S2P6-H429F mAb and S2P6-WT mAb showed near identical binding properties. Similarly, the CC40.8-H429F and CV3-25-H429F mAbs showed very similar binding profiles to their respective wild type counterparts, indicating that the H429F modification of the heavy chain does not affect interaction of the mAbs with the SARS-CoV-2 spike antigen.

The CDC potency of the S2P6-H429F and CC40.8-H429F mAbs, relative to their wild type counterparts S2P6-WT and CC40.8-WT, was also determined (see FIG. 49D). It was found that the S2P6-WT mAb showed no CDC of Ramos-S cells at any concentration above the background control of lysis in the absence of antibody but in the presence of only complement (i.e. no mAb C′ only; FIG. 49D). However, the S2P6-H429F mAb showed potent killing. Similarly, the CC40.8-H429F mAb also showed readily detectable killing above background though not as great as the potency of S2P6-H429F.

Modification of the CH3 Domain Enables and Fusion Proteins and mAbs of Distinct Specificity to Act Synergistically in CDC

Combinations of the ACE2-Fc fusion protein and anti-SARS-CoV-2 mAbs were investigated for potential synergy leading to greater functional potency (i.e. by determination of enhanced CDC lysis of targets) (see FIG. 50A and FIGS. 50B). In particular, cooperation in CDC between the fusion protein (which binds with the RBD of the spike protein) and anti-SARS-CoV-2 mAbs (which detect epitopes in the stem region of the spike protein), was determined using pairwise combinations of flACE2-Fc with the various anti-SARS-CoV-2 mAbs.

The combination of flACE2-Fc-WT with the S2P6-WT mAb failed to show any CDC above background. Moreover, no lysis was detected when the S2P6-WT was titrated in the presence of a fixed concentration (1 μg/ml) (flACE2-Fc-WT; FIG. 50A). In contrast, functional cooperativity was readily apparent between the flACE2-Fc-H429F fusion protein and the S2P6-H429F mAb (FIG. 50A). The S2P6-H429F mAb, when titrated alone, mediated potent CDC of Ramos-S cells in the absence of flACE2-Fc-H429F. The flACE2-Fc H429F at the limiting concentration of 1 μg/ml, mediated detectable CDC (23.43% killing). Remarkably, the level of CDC mediated at all concentrations of S2P6-H429F in the presence of a fixed concentration (1 μg/ml) of flACE2-Fc-H429F, was considerably greater than with the S2P6-H429F mAb alone or of the flACE2-Fc-H429F alone (FIG. 50A). The cooperation between the flACE2-Fc-H429F fusion protein and the S2P6-H429F mAb was also readily detected when the concentration of the mAb was limiting (0.03 μg/ml 0.125 μg/ml) shown by the arrows in FIG. 50A. The cooperation was also evaluated between flACE2-Fc-H429F and two additional anti-SARS-CoV-2 spike mAbs, namely CC40.8 and CV3-25 (FIG. 50B). CDC was determined for the mAbs alone (at 2.5 μg/ml of mAb), flACE2-Fc-H429F alone and in pairwise mixtures. Increased CDC was evident in the combination of the flACE2-Fc-H429F fusion protein and the CC40.8-H429F mAb compared to the CDC mediated by either alone. Similarly, the combination of flACE2-Fc-H429F with the CV3-25-H429F mAb mediated an increased level of CDC compared to the killing by either alone (FIG. 50B). Clearly, the surprising cooperation between the fusion protein and monoclonal antibodies can occur broadly and is not restricted to a single combination of proteins. More remarkable is that such cooperation was achieved between two proteins of distinct molecular formats (i.e. a fusion protein comprising a receptor (ACE2) fused to an Fc region component with a H429F modification synergistically cooperating with a monoclonal antibody to achieve greater functional potency).

### Example 13 H429F in an ACE2-Fc Protein Confers CDC Independent of the ACE2 Format

Experimentation was conducted to determine the effect of H429F modification on CDC potency using ACE2-Fc fusion proteins comprising structurally distinct forms of the ACE2 polypeptide. In particular, three ACE2-Fc fusion proteins were evaluated wherein the ACE2 component comprised either the truncated ACE2 ectodomain comprising only the catalytic domain of the ectodomain and excluding the collectrin domain (trACE2-Fc), or the full length ACE2 ectodomain (flACE2-Fc) or the enhanced full length ACE2 ectodomain comprising a triple mutation within the ACE2 polypeptide that has been reported to improve its binding affinity to the receptor binding domain (RBD) of SARS-CoV-2 spike protein (EflACE2-Fc) (Chan et al., 2020 supra).

### Methods and Materials

The ACE2-Fc fusion proteins (trACE2-Fc, flACE2-Fc and EflACE2-Fc) comprising the different structural forms of the ACE2 polypeptide were generated with either a wild type Fc region component or an Fc region component including a H429F mutation substantially as described above in Example 1. CDC potency was evaluated by flow cytometry using the Zombie Green method and using a 1/4 dilution of human serum as a source of complement.

### Results and Discussion

The binding of the ACE2-Fc fusion proteins to Ramos-S cells expressing the SARS-CoV-2 spike protein was determined by flow cytometry and the results are shown in FIG. 51. The trACE2-Fc-H429F and its wild type counterpart, trACE2-Fc-WT, showed near identical binding to SARS-CoV-2 spike protein (5.1 nM and 5.3 nM respectively; FIG. 51A). Similarly, flACE2-Fc-H429F and EflACE2-Fc-H429F showed near identical binding to their wild type counterparts flACE2-Fc-WT and EflACE2-Fc-WT (FIGS. 51B and 51C respectively). Thus, H429F modification does not significantly impact on the strength of binding of the fusion proteins to the target protein SARS-CoV-2 spike.

The EC50 values showed that the affinity enhanced EflACE2-Fc fusion proteins with either a wild type Fc region component or an Fc region component with the H429F mutation, showed the greatest level of binding to Ramos-S cells. Interestingly, the trACE2-Fc fusion protein, which includes only the ACE2 catalytic domain and lacks the collectrin domain found in the flACE2, showed stronger binding (EC₅₀ 4.4 nM) to SARS-CoV2 spike than the flACE2-Fc fusion proteins (EC₅₀ 6.6 nM) (FIG. 51).

Despite the near identical binding of each set of ACE2-Fc fusion proteins, CDC was greatly enhanced when the Fc region component included the H429F mutation (FIG. 52). Thus, trACE2-Fc-H429F (FIG. 52A), flACE2-Fc-H429F (FIG. 52B) and EflACE2-Fc-H429F (FIG. 52C) mediated potent CDC but, in contrast to their wild type counterparts, trACE2-Fc-WT, flACE2-Fc-WT and EflACE2-Fc-WT which mediated little or no CDC.

The enhancement of CDC achieved by the H429F modification of ACE2-Fc fusion proteins was independent of the nature of the ACE2 polypeptide as all of the ACE2-Fc-H429F forms mediated potent CDC. Interestingly, the hierarchy of CDC potency observed was that EflACE2-Fc-H429F was more potent than trACE2-Fc-H429F, which was more potent than flACE2-Fc-H429F. It is noteworthy that ELISA experiments investigating C1q binding (FIG. 11) by ACE2-Fc fusion proteins also demonstrated that trACE2-Fc-H429F showed a greater capacity to bind C1q and form the membrane attack complex (C5b-9) compared to flACE2-Fc-H429F, thereby indicating that enhancement of C1q binding correlates with the relative CDC potency of fusion proteins, in this case ACE2-Fc. An extension of this observation is that the quantitation of C1q binding to H chains or Fc region components, or of the formation of the membrane attack complex, such as by using ELISA-based methods, may provide a rapid screen for the selection of antibodies, antibody-like molecules, Fc-fusion proteins and the like for their potential to mediate CDC and thus, for the discrimination between the levels of CDC killing potency mediated by such proteins.

### Example 14 Production of an IgG-Like ACE2-Fc Fusion Protein

This example sought to produce an immunotherapeutic protein (comprising an Fc region component with an amino acid substitution at position H429 of the amino acid sequence of human IgG1 heavy chain) having a different structure to those of the preceding examples. That is, the immunotherapeutic protein in this case may be considered as an example of an antibody-like (Ab-like) molecule; more particularly, an Ab-like fusion protein.

Ab-like proteins are based on the archetypal immunoglobulin structure (see FIG. 2 and FIG. 53A), but have one or more target recognition structure (not of immunoglobulin origin; that is, not a typical VL or VH domain) replacing the variable domains of the heavy and light chains. In this example, the affinity enhanced full length ACE2 polypeptide (EflACE2) was used in the production of an EflACE2-Ab-like fusion protein comprising a polypeptide complex assembled into an H₂L₂-like protein, wherein there are two heavy chain-like polypeptides each comprising EflACE2 linked to an Fc region component (i.e. an H chain) and additionally, two light chain-like polypeptides each comprising EflACE2 linked to a constant (C) domain of a light chain shown. A diagrammatical representation of this molecule is shown in the middle panel of FIG. 53A.

### Methods and Materials

### Constructs

The mature EflACE2-Ab-like-WT fusion protein used in this example comprised a heavy chain polypeptide shown as SEQ ID NO: 104 comprising the EflACE2 ectodomain comprising the entire ectodomain fused, in the order the N-terminus to the C-terminus, to CH1-hinge-CH2-CH3 domains of wild type human IgG1, and is encoded by the codon-optimised polynucleotide sequence shown as SEQ ID NO: 105. Similarly, the mature polypeptide of the light chain shown as SEQ ID NO: 106 comprised the EflACE2 ectodomain fused to the N-terminus of a human kappa constant domain, and is encoded by the codon-optimised polynucleotide sequence shown as SEQ ID NO: 107. The mature EflACE2-Ab-like-H429F fusion protein comprised a heavy chain polypeptide with an amino acid sequence shown as SEQ ID NO: 108 with an Fc region component with the H429F modification, and is encoded by the codon-optimised polynucleotide sequence shown as SEQ ID NO: 109.

The EflACE2-Ab-like-WT, comprising an IgG1-WT H chain, expression construct was assembled using NEBuilder (New England Biolabs) by the appropriate insertion of a synthetic DNA fragment encoding linking sequence and a human IgG1-CH1 domain (GeneArt; Thermo Fisher Scientific) into the EflACE2-Fc-WT expression vector cleaved at the sequence encoding the linker between the EflACE2 and Fc region component sequences. In a similar approach, the EflACE2-Fc-WT expression vector was cleaved to excise the fragment encoding the Fc region component sequences and a synthetic DNA encoding a linking sequence and a human constant light kappa domain was incorporated. The mutated H429F H chain variant was produced as described in Example 1. Thus, the expression constructs consisted of several synthetic polynucleotide sequences that, joined together, encoded the EflACE2-Ab-like fusion protein heavy chain or light chain, within the expression plasmid pcDNA3.4 (Thermo Fisher Scientific).

### Expression and Purification

Expression of the Ab-like fusion proteins was conducted using transient transfection of Expi293F cells (Thermo Fisher Scientific) using the conditions described hereinbefore in Example 1.

The fusion proteins were purified to homogeneity by Protein A affinity chromatography under gentle elution conditions according to the following protocol:
(i) On Day 1, the transfected Expi293F cells were incubated at 37 °C After the addition of enhancers, the cells were then incubated at 34 °C, 125 rpm, and 8% CO₂ atmosphere. Six days after transfection, the supernatant was harvested by centrifugation at 3000×g for 15 minutes. The supernatant was collected into a fresh tube and spun again for an additional 30 minutes. The supernatant was then filtered through a 0.22 μm filter.
(ii) The EflACE2-Ab-like-H429F fusion protein was purified using a 1 mL Hitrap™ Protein A column (Cytiva Life Sciences, Marlborough, MA, United States of America), with all steps at a flowrate of 1 ml/min. In a preferred method, the column was first equilibrated with 20 mM phosphate buffer, pH 7.0. The filtered supernatant was loaded onto the Protein A column using a BioLogic LP low-pressure chromatography system (Bio-Rad, Hercules, CA, United States of America). After loading was completed, the column was washed with 20 Column Volumes (CV) of 20 mM phosphate buffer pH 7.0. The column was further washed with 5CV of Buffer A—30 mM Arginine pH 4.0. The bound proteins were eluted using a shallow linear gradient to 35% buffer B—130 mM arginine, pH 4.0 performed over 20 CV. Fractions of 1 mL were collected, neutralised using 1M Tris-HCl pH 9.0, and concentrations determined using the 0.1% (mg/ml) extinction coefficient at 280 nm of 1.80 (derived using protparam at https://web.expasy.org/protparam/). Fractions 15, 20, 24, 25 and 27 across the elution peak were analysed using size exclusion chromatography (SEC). Because of the gentle elution conditions of this shallow arginine gradient, all of these tested fractions (i.e. 15, 20, 24, 25 and 27) showed approximately the same monomer to oligomer ratio of ~85:15% and were free of large molecular weight aggregates. Thus, all fractions (8-34 in FIG. 54A) of the EFlACE2-Ab-like fusion protein eluted from the Protein A column were pooled and concentrated using a 30 kDa cut-off filtration device (Merck) and separated by SEC using a Superose 6 Increase 10/300 GL column (Cytiva Life Sciences). The pooled and concentrated EflACE2-Ab-like fusion protein eluted from the Protein A column had a similar SEC profile (FIG. 54B) to that of the individual fractions 15, 20, 24, 25 and 27 (data not shown). SDS-PAGE analysis indicated both the Protein A pooled eluate and the SEC-derived monomeric fraction comprised a heavy (H) and light (L) chain on reduction, and under non-reducing conditions comprised a fully disulfide-bonded, high molecular weight species consistent with a H₂L₂ composition (FIG. 54C).

### Complement Dependent Cytotoxicity (CDC) Assay and Spike Protein Binding

CDC potency was evaluated by flow cytometry using the Zombie Green method in a similar manner to the CDC assay described in Example 1. Binding of the EflACE2-Ab-like proteins to SARS-CoV-2 spike protein was evaluated by flow-cytometry using Ramos-S cells as described herein in Example 1.

### Results and Discussion

Production and purification of the Ab-like fusion proteins As described above, ACE2-Fc fusion proteins in Example 1 can be appropriately purified by sequential ion exchange (IEX) chromatography and size exclusion chromatography (see FIG. 5). An alternative to the use of the IEX step for the purification for the Ab-like proteins described in this example, is the use of Protein A affinity chromatography which is a highly specific affinity purification and is appropriate for at-scale production in an industrial setting, and with the use of arginine in the conditions for elution of the proteins from the Protein A column, aggregate formation in the eluted proteins may be suppressed (see, for example, Arakawa T et al., Protein Expr Purif 36(2):244-248, 2004; 2004 et al., Ejima D et al., Anal Biochem 345(2):250-257, 2005; and Shukla D et al., J Phys Chem B 115(11):2645-2654, 2010). Purification of ACE2-fusion proteins containing an Fc region component used elution by a low concentration of arginine, less than 130 mM, and at less than or equal to pH 5. The appropriate low arginine concentration can be achieved by standard liquid chromatography systems that can deliver a buffer gradient to the column. For example, in a preferred method of purifying EflACE2-Ab-like-H429F, purification is achieved through binding to Protein A under standard conditions followed by washing with five column volumes of Buffer A —30 mM arginine (pH 4.0) and elution with a linear gradient to 35% of 130 mM arginine (pH 4.0). Elution under other conditions, for example 100 mM sodium citrate buffer pH 3.0, may result in the elution of ACE2-Fc or ACE2-Ab-like fusion proteins containing some aggregates.

However, this preferred method of the use of arginine under the disclosed conditions herein, yielded EflACE2-Ab-like protein from the Protein A chromatography that was demonstrated to be highly pure by SDS-PAGE, and was also largely monomeric (~85%) as determined by SEC (i.e. relatively free of aggregates). Particularly, FIG. 53B shows that the EflACE2-Ab-like-WT protein was obtained as a pure, fully disulfide-bonded H₂L₂ protein that, on reduction with dithiothreitol, generated equal amounts of EflACE2-H and EflACE2-L chains. FIG. 54C shows that the EflACE2-Ab-like-H429F protein was also obtained as a pure, fully disulfide-bonded H₂L₂ protein that, on reduction with dithiothreitol, generated equal amounts of EflACE2-H and EflACE2-L chains.

### Target Recognition and CDC Potency of Ab-Like Fusion Protein

The CDC potency of the EflACE2-Ab-like-WT and EflACE2-Ab-like-H429F fusion proteins was determined by flow cytometry analysis (FIG. 53C). The EflACE2-Ab-like-WT failed to mediate detectable CDC killing of Ramos-S cells—indeed, lysis was similar to the background lysis (5.1%) of the cells in the complement only control. In contrast, the EflACE2-Ab-like-H429F fusion protein showed that it mediated powerful CDC killing of the SARS-CoV-2 spike expressing Ramos-S cells. These differences in CDC potency were not attributable to differences in binding of targets by the WT and H429F forms of the proteins. Analysis by flow cytometry, of the binding of the EflACE2-Ab-like proteins to Ramos-S cells expressing SARS-CoV-2 spike protein demonstrated that the EflACE2-Ab-like-WT and the EflACE2-Ab-like-H429F showed comparable levels of binding to the cells (FIG. 53D).

Clearly the modification of H429 of the heavy chain enhanced the CDC function of the Ab-like proteins. Thus, modification at position 429 of the immunoglobulin-H chain (e.g. mutations such as H429F or H429Y) are versatile modifications that enhance the functional utility of different classes of polypeptides that are derived from immunoglobulins (i.e. the function enhancing effects mediated by H429 mutation are apparent in antibodies, antibody-like proteins and in Fc-fusion proteins). Moreover, while the EflACE2-Ab-like fusion protein of this example comprises four polypeptide chains all of which comprise the same target recognition structure (i.e. EflACE2), it is considered that an Ab-like fusion protein may otherwise comprise heterologous polypeptides such that may each comprise a different target recognition structure (i.e. if formatted as an H₂L₂ complex, the Ab-like protein may comprise 1, 2, 3 or 4 different target recognition structures depending on the combinations of H and L chains comprising the different target recognition structures; see FIG. 53A, right panel). Additionally, Ab-like proteins formatted in an H₂ configuration (i.e. without associated light chains) may comprise the same or different target recognition structures in the two chains. The H chains of such molecules may be modified by mutation of H429 (e.g. H429F) to alter their effector responses in the same way as the CDC potency of EflACE2-Ab-like-H429F was enhanced here. Finally, it may be noted that the large size of the EflACE2-Ab-like-H429F molecule (>500 kDa) might have been predicted to impose steric restraints on the function of the Fc portion, even with the H429F mutation, but clearly this was not the case and indeed H429F mutation conferred potent CDC and can therefore be considered as representing a rigorous example of the capacity of the H429F modification of an Fc region component to confer improved effector function in Fc-fusion proteins.

### Example 15 H429F Modification Confers Enhanced Function in Antibodies with Mutations in the CH2 Domain Inactivating FcγR Binding and/or Complement Dependent Effector Function

H429F modification of the Fc region component was further investigated in heavy chains comprising additional mutations that alter the capacity of an antibody to activate immune effector functions, such as Fc receptor binding and/or complement dependent functions. In particular, the antibodies in this example included modifications to the hinge or the CH2 domain of the IgG H chain known to increase or decrease immune effector function (see Wang et al., 2018 supra; and Lee C-H et al., Nat Immunol 18:889-898, 2017). Such modifications can confer properties broadly, for example, on all FcγR functions as well as complement activation or confer selective properties on the antibody such as enhancement or impairment of function of individual FcγR or of selectively altered complement function. It is important to note that these modifications in the hinge or CH2 domain of antibody (e.g. IgG) are distant from the CH3 domain that includes the H429. Since a feature of an immunoglobulin H chain is the modular domain structure, it is possible that the properties conferred on Ig H chains or Fc region components by H429 modification may be unaffected by mutations in CH2 domain that affect immune effector functions such as FcγR binding or complement activation or antibody half-life relating to FcRn binding (Dall'Acqua W F et al., J Biol Chem 281:23514-23524, 2002). That is, functional properties of the H429 modified Ig-heavy chain containing molecules such as antibodies, Ab-like molecules and fusion proteins may be further modified for enhanced or reduced functions by mutations in, for example, the CH1, the hinge or CH2 regions.

Alteration of immune effector function of IgG can be achieved by mutation of the IgG residues L234 and/or L235 (Eu numbering) which are located in the hinge proximal region of CH2 also known as lower hinge. One of the most widely used mutational strategies to inactivate immune effector functions mediated by IgG1 is the substitution of these residues with alanine (i.e. L234A and L235A) also known as LA/LA (Wang et al., 2018 supra). The L234A, L235A mutations inactivate interactions with all cell surface FcγR and similarly inactivate the antibody-dependent complement cascade (Chenoweth et al., 2020 supra, and Wang et al., 2018 supra). In this example, the effect of combining in the one H chain, a modified CH2 domain with a modified CH3 domain including the H429F modification was examined. In particular, death receptor mAbs BDR5 and TDR5 were formatted on human IgG1 backbone and synthesised such that each H chain of the mAbs contained the L234A, L235A and H429F mutations (i.e. referred to herein as BDR5-1LA/LA/HF and TDR5-1LA/LA/HF) and evaluated for effects on signal amplification in assays of apoptosis.

### Methods and Materials

### Antibodies and Antibody Constructs

The mAbs used in this example are based on the sequences of two distinct mAbs which specifically bind to the human DR5 molecule as described in Example 8. The DR5-specific H chain of the BDR5-1LA/LA/HF mAb comprised H chains of the human IgG1 subclass containing the L234A, L235A and H429F mutations and lacking the C-terminus lysine (delK447). Its amino acid sequence is shown as SEQ ID NO: 110, and is encoded by the codon-optimised polynucleotide sequence shown as SEQ ID NO: 111. The DR5-specific kappa light chain of BDR5-1LA/LA/HF mAb comprises the DR5-specific kappa light chain of the BDR5 mAb described in Example 8 and the polypeptide and encoding polynucleotide sequences are shown as SEQ ID NO: 48 and 49 respectively. Turning to the DR5-specific H chain of the TDR5-1LA/LA/HF mAb, this is comprised of H chains of the human IgG1 subclass containing the L234A, L235A and H429F mutations and lacking the C-terminus lysine (delK447), its amino acid sequence is shown as SEQ ID NO: 112, and is encoded by the codon-optimised polynucleotide sequence shown as SEQ ID NO: 113. The DR5-specific kappa light chain of TDR5-1LA/LA/HF mAb comprises the DR5-specific kappa light chain of the TDR5 mAb as described in Example 8; the polypeptide and encoding polynucleotide sequences are shown as SEQ ID NO: 54 and SEQ ID NO: 55 respectively. The other mAbs used herein comprising WT IgG1(delK447) H chains (i.e. BDR5-1WT and TDR5-1WT) or comprising H429F modified IgG1 chains (delK447) (i.e. BDR5-1H429F and TDR5-1H429F) were as described above in Example 8.

### Synthesis of Unmodified and Mutated Heavy Chains

Antibody expression constructs and Fc variants including the replacement of H429 with phenylalanine (H429F), the replacement of L234 with alanine and of L235 with alanine (L234A, L235A or LA/LA) were generated by standard methods known to those skilled in the art and are as described elsewhere in this specification in relation to therapeutic mAbs such as in Example 3.

### Expression of Antibody Constructs

Expression of the antibodies was conducted using transient transfection of Expi293F cells (Thermo Fisher Scientific) under the same conditions as has been described above. As for all mAbs, the cell cultures were harvested after transfection and centrifuged at 2500 rpm for 20-30 minutes, and the supernatant filtered with a 0.2 μm high flow filter (Sartorius AG) prior to purification.

Purification of mAbs and Evaluation of Antigen Binding

The BDR5-1LA/LA/HF and TDR5-1LA/LA/HF mAbs were purified from supernatant of the transfected Expi293F cells using Protein A affinity chromatography as described above (e.g. Example 8) and the mAbs showed the expected H₂L₂ format of monomeric antibodies as confirmed by SDS-PAGE analysis.

### Viability Assay

The capacity of antibodies to induce cell death was evaluated in a viability assay as described in Example 8. The antibodies were tested either individually as single agents or as mixtures of two antibodies. The viability assay was performed using Colo205 cells which were cultured routinely in culture medium consisting of RPMI 1640 medium supplemented with foetal calf serum (10% v/v) plus 4 mM glutamine and 100U/ml penicillin and 0.1 mg/ml streptomycin. The cells were harvested from cell culture, washed in culture medium by centrifugation at 200×g and resuspended in culture medium. The cells were then washed again and resuspended in culture medium to a concentration of 1.2×10⁶/ml. One hundred microlitres (100 μL) of cell suspension (10,000 cells) was added to the wells of 96-well flat-bottom sterile plates. Where the mAbs were used in mixtures, the total concentration of mAb was the same as in the equivalent wells treated with the single antibodies. The plates were then incubated for 48 hours at 37 °C in 5% CO₂. The maximum viability control values were determined from cells cultured in 200 μl of culture medium in the absence of antibody. The control for maximum death was determined from wells seeded only with cell suspension in 200 μl of culture medium but treated in the last two hours of the incubation period (i.e. at the end of the incubation period) with 10 μl of 1% SDS. Following the incubation period, the viability of the cells was determined using a colorimetric assay using Cell Counting Kit 8 (CCK-8 using the tetrazolium salt, WST-8) (Abcam) and the plates incubated at 37 °C for 2-5 hours. Colour development was determined by measuring absorbance at 450 nm (Abs450) on a Fluorostar OPTIMA (BMG).

### Results and Discussion

The effect of H429 modification on the apoptosis killing potency of mAbs comprising heavy chains with additional mutations was determined. The BDR5-1LA/LA/HF mAb and the TDR5-1LA/LA/HF mAb were used individually and were also used in combination (BDR5-1LA/LA/HF+TDR5-1LA/LA/HF) (see FIG. 55). It was clear that when used individually, the BDR5-1LA/LA/HF mAb mediated a reduction in survival of the Colo205 cells compared to survival in the absence of antibody. Similarly, the TDR5-1LA/LA/HF mAb reduced cell survival when used individually. The greatest killing potency was obtained when the mAbs were used in combination which achieved levels of killing that were equivalent to the control for maximum death of cells determined by cell survival in the presence of SDS detergent. Even at concentrations as little as 0.6 μg/ml, the mixture of BDR5-1LA/LA/HF and TDR5-1LA/LA/HF mAbs mediated near complete cell death (FIG. 55). It is important to note that the cell death induced by the mixture of BDR5-1LA/LA/HF and TDR5-1LA/LA/HF mAbs which lack immune effector function, is comparable to the killing potency of the mixture of BDR5-1HF and TDR5-1HF (FIG. 55) which contain only the H429F mutation and retain the unmutated L234 and L235 residues.

The increased apoptosis achieved by the BDR5-1LA/LA/HF and TDR5-1LA/LA/HF mAbs when used alone is also consistent with the increased apoptosis by BDR5-1HF and TDR5-1HF mAbs, when used as single agents (see FIG. 38 and FIG. 42) where the BDR5-1HF and TDR5-1HF mAbs mediated improved apoptosis when formatted either on IgG1 or IgG2 backbone. Thus, the LA/LA/HF variants would likewise be expected to also mediate enhanced function on IgG2 backbones.

It is clear that the induction of apoptosis death by anti-DR5 antibodies either used alone or in combination is conferred or enhanced by modification of H429 (FIG. 55, and also FIG. 38 and FIG. 39) and the most potent apoptosis is achieved when the mAbs are used in combination. Importantly, the H429F enhancement of apoptosis was retained despite the presence of mutations elsewhere in the heavy chain, which in this case are known to inactivate both Fc receptor activation and complement activation (i.e. the effects of CH2 modification is independent of the effects of H429 modification). Thus, modifications of CH2 which impact these functions do not affect the unique oligomerisation properties conferred on an IgG Fc region by H429F modification which, in this case, is manifest by the enhanced signalling via DR5 induced by H429F mutation resulting in enhanced apoptotic cell death.

In addition to the LA/LA mutations that impair effector functions of IgG, other mutations in CH2 are well known to alter effector function (An Z et al., MAbs 1:572-579, 2009) including mutation of N297 of the IgG heavy chain which eliminates N-glycosylation of the IgG heavy chain and thereby inactivates effector functions (Walker M R et al., Biochem J 259:347-353, 1989). Additionally, effector functions have been modified in the other human IgG subclasses by mutation of their lower hinge sequences —IgG3 is identical to IgG1 (LLGG) and LA/LA inactivates its effector function. Moreover, IgG2 and IgG4 (An Z et al., 2009 supra; and Xu D et al., 2000 supra), which already show reduced effector function and an impaired capacity to activate the complement cascade, can also be mutated to inactivate their immune effector functions (Wang X et al., 2018 supra). Thus, the data showing the function enhancing effects of H429 mutation and the effects resulting from alterations to CH2 to affect immune effector function together indicate that H429 modification of CH3 could also be applied with modifications in CH2 that increase function such as increasing FcγR activation (Lazar G A et al., 2006 supra) or complement activation (Lee C-H et al., 2017 supra; and Wang X et al., 2018 supra) or both or FcRn binding (Dall'Acqua W F et al., 2016 supra). Further, while in this example, mutations were made in separate domains, multiple mutations can frequently be made in the same domain of the immunoglobulin chain with the resulting mutant displaying properties conferred by each of the individual mutations. For example, in some embodiments, an H429 substitution (i.e. in CH3) may be combined with modifications known to enhance binding to FcRn (e.g. M438L and N434S; Zalevsky J et al., 2012 supra), that are also in CH3, so as to increase the in vivo half-life of an immunotherapeutic protein of the disclosure. Further, while in this example, the effects of the various mutations on signal amplification were evaluated by apoptosis, it is to be understood that enhancement of signal amplification may also be observed by, for example, other means or outcomes from increased signalling-dependent biological effects on the cell, such as cellular responses (including receptor-dependent cellular responses such as cell activation), inhibition of cell activation or function, and cytokine release.

### Example 16 Chimeric Anti-TNP IgG Antibodies Comprising Mutation and Activity Analysis

### Methods and Materials

Characteristics of the antibodies and any mutations and their SEQ IDs are detailed in Table 5. Note that the notation for mutation used throughout this example is as follows: the name of the amino acid is shown as it name or as a one letter code or three letter code and its position in the H chain is given on the basis of the Eu numbering convention of human IgG1 and the sequence alignment shown in FIG. 56. Thus for example the PVA amino acids sequence the single letter denotes the amino acid and further noting that the “⋅” in that sequence denotes a gap in the sequence alignment of the IgG2 H chain as shown in FIG. 56. The numbers associated with amino acid positions for example 233-236 define the position of the amino acids in the in the heavy chain according to the Eu numbering convention as shown in FIG. 56. Thus for IgG2 H chain P233, −234, V235, A236 represents proline (P) found at positions 233, the “⋅” represents a gap in the sequence alignment at position 234, valine (V) is found at position 235, and alanine (A) is found at position 236. By way of example if the sequence PVA amino acids were replaced by the equivalent sequence from IgG1 being the amino acids glutamic acid (E) at position 233, leucine (L) at position 234, leucine (L) at position 235, and glycine (G) 236. Such description may be also represented as PVA(233-236)ELLG. Similarly for example a point mutation may be referred to as H429 to H429F or H(429F). This convention is used throughout.

### Production of Anti-TNP Human IgG and Mutant Anti-TNP Human IgG Plasmid Constructs

### (a) Construction of Anti-TNP Mabs Comprising Unmodified, Wild-Type (VT) Human H Chains

Chimeric anti-TNP human IgG antibody constructs consisting of the variable heavy (V_{H}) and light (V_{L}) region sequence of the mouse anti-trinitrophenyl (anti-TNP) antibody TIB142 and the sequence from the constant heavy (CH) region from human IgG subclasses have been described previously in detail hIgG2 and hIgG4 (Wines et al., 2016 J Immunology 2016 197:1507-1516; doi: 10.4049/jimmunol.1502551). All chimeric antibody sequences were subcloned into the pCR3vector.

Thus, the TNP-WT mAbs used in this example comprised either of two human heavy chain isotypes; first, an IgG1 isotype polypeptide (SEQ ID NO: 32) comprising, in the order of the N-terminus to the C-terminus, the TNP-specific V_{H} domain of mouse monoclonal antibody TIB142 fused to CH1-hinge-CH2-CH3 domains of human IgG1, encoded by the cDNA (SEQ ID NO: 33) (Patel D et al., J Immunol 184: 6283-6292, 2010); An IgG2 isotype polypeptide (SEQ ID NO: 34) comprised, in the order of N-terminus to the C-terminus, the TNP-specific V_{H} domain of mouse monoclonal antibody TIB142 fused to CH1-hinge-CH2-CH3 domains of human IgG2, and encoded by the DNA (SEQ ID NO: 35).

An IgG4 isotype polypeptide (SEQ ID NO: 114) comprised, in the order of N-terminus to the C-terminus, the TNP-specific V_{H} domain of mouse monoclonal antibody TIB142 fused to CH1-hinge-CH2-CH3 domains of human IgG2, encoded by the codon-optimised cDNA with the sequence shown as SEQ ID NO: 115.

The TNP-specific light chain polypeptide (SEQ ID NO: 36) comprised the TNP-specific V_{L}domain of mAb TIB142 fused to the human kappa chain constant domain, encoded by the codon-optimised DNA with the sequence shown as SEQ ID NO: 37.

(b) mAbs Comprising Mutated Human IgG2 H Chains

A series of IgG2 based anti-TNP mAbs were generated comprising H chains wherein amino acid changes had been made as shown in Table 5 and described as follows:

The mAb TNP2x5 comprised a modified IgG2 heavy chain (SEQ ID NO: 116) wherein the lower hinge amino acid sequence PV-A at position 233-236 of the wild-type IgG2 heavy chain (Eu numbering, FIG. 56 and Table 9) was replaced with the amino acids EFLG derived from the lower hinge of human IgG4 at positions 233-236 (PVA(233-236)EFLG) and was encoded by a codon-optimised cDNA. (SEQ ID NO: 117).

The mAb TNP2x4 comprised a modified IgG2 heavy chain amino acid sequence (SEQ ID NO: 118) wherein the lower hinge amino acid sequence PV-A at position 233-236 of the wild-type IgG2 heavy chain (Eu Numbering, FIG. 56 and Table 9) was replaced with the amino acids ELLG derived from the lower hinge of human IgG1 at positions 233-236 (PVA(233-236)ELLG) and was encoded by a codon-optimised cDNA. (SEQ ID NO: 119).

The mAb TNP2x15 comprised a modified IgG2 heavy chain amino acid sequence (SEQ ID NO: 120) wherein the lower hinge amino acid sequence PV-A at position 233-236 of the wild-type IgG2 heavy chain (Eu Numbering see FIG. 56 and Table 9) was replaced with the amino acids EFEG (PVA(233-236)EFEG) and which additionally comprised a point mutation within the CH3 domain at position 445 (P445L) wherein proline (P) was replaced by leucine (L) and was encoded by a codon-optimised cDNA (SEQ ID NO: 121). The EFEG amino acid sequence incorporated into the IgG2 H chain differs from the sequence of the human IgG4 lower hinge (EFLG) by virtue of a single amino acid replacement of the leucine (L) with glutamic acid (E).

The mAb TNP2x17 comprised a modified IgG2 heavy chain amino acid sequence (SEQ ID NO: 122) wherein hinge amino acid sequence RKCCVE at position 217-225 of IgG2 was replaced with PKSCDKTHT of IgG1 (RKCCVE(217-225)PKSCDKTHT) and which additionally comprised a point mutation with the CH3 domain at position 445 (P445L) and was encoded by a codon-optimised cDNA (SEQ ID NO: 123)

(c) antiTNP mAbs Comprising Mutated IgG1 H Chains

A series IgG1 based anti-TNP mAbs were generated comprising H chains wherein amino acid changes had been made as shown in Table 5 and described as follows:

The mAb TNP1x32 comprised modified IgG1 heavy chain amino acid sequence (SEQ ID NO: 124) containing the mutation EPKSCDKTHT(216-225)ERKCCVE and was encoded by a codon-optimised cDNA (SEQ ID NO: 125).

The mAb TNP1x3 comprised modified IgG1heavy chain amino acid sequence (SEQ ID NO: 126) containing a point mutation within the CH3 domain at position 429 (H429F) wherein the amino acid histidine (H) was replaced with phenylalanine (F) and was encoded by a codon-optimised cDNA (SEQ ID NO: 127).

The mAb TNP1x5 comprised modified IgG1 heavy chain amino acid sequence (SEQ ID NO:128) containing a point mutation within the CH3 domain at position 430 (E430G) wherein the amino acid glutamic acid (E) was replaced with glycine (G) and was encoded by a codon-optimised cDNA (SEQ ID NO: 129).

The mAb TNP1x1 comprised modified IgG1 heavy chain amino acid sequence (SEQ ID NO: 130) containing a point mutation within the CH3 domain at position 429 (H429Q) wherein the amino acid histidine (H) was replaced with glutamine (Q) and was encoded by a codon-optimised cDNA (SEQ ID NO: 131).

The mAb TNP1x2 comprised modified IgG1 heavy chain amino acid sequence (SEQ ID NO: 132) containing a point mutation within the CH3 domain at position 429 (H429E) wherein the amino acid histidine (H) was replaced with glutamic acid (E) and was encoded by a codon-optimised cDNA (SEQ ID NO: 133).

The mAb TNP1x9 comprised modified IgG1 heavy chain amino acid sequence (SEQ ID NO: 134) containing a point mutation within the CH3 domain at position 435 (H435A) wherein the amino acid histidine (H) was replaced with alanine (A) and was encoded by a codon-optimised cDNA (SEQ ID NO: 135).

The mAb TNP1x10 comprised modified IgG1 heavy chain amino acid sequence (SEQ ID NO: 136) containing a point mutation within the CH1 domain at position 131 (S131C) wherein the amino acid serine (S) was replaced with cysteine (C) and was encoded by a codon-optimised cDNA (SEQ ID NO: 137).

The mAb TNP1x14 comprised modified IgG1 heavy chain amino acid sequence (SEQ ID NO: 138) containing a point mutation within the CH1 domain (S131C) and additionally comprised the hinge mutation EPKSCDKTHT(216-225)ESKYGPP and was encoded by a codon-optimised cDNA (SEQ ID NO: 139).

The mAb TNP1x15 comprised modified IgG1 heavy chain amino acid sequence (SEQ ID NO: 140) containing mutation at positions 295, 296 and 297 within the CH2 domain (QYN(295-297)NST) and was encoded by a codon-optimised cDNA (SEQ ID NO: 141). This mutation results in a relocation of the N-linked glycan of the heavy chain.

The mAb TNP1x12 comprised modified IgG1 heavy chain amino acid sequence (SEQ ID NO: 142) containing mutation at positions 296, 297 and 298 within the CH2 domain (YNS(296-298)NSS) and was encoded by a codon-optimised cDNA (SEQ ID NO: 143). This mutation results in a relocation of the N-linked glycan of the heavy chain.

The mAb TNP1x16 comprised modified IgG1 heavy chain amino acid sequence (SEQ ID NO: 144) containing mutation at positions 269 and 270 within the CH2 domain, (ED(269, 270)AA) and was encoded by a codon-optimised cDNA (SEQ ID NO: 145).

The mAb TNP1x22 comprised modified IgG1 heavy chain amino acid sequence (SEQ ID NO: 146) containing a point mutation at positions 429 within the CH3 domain, (H429S) and was encoded by a codon-optimised cDNA (SEQ ID NO: 147).

The mAb TNP1x23 comprised modified IgG1 heavy chain amino acid sequence (SEQ ID NO: 148) containing a point mutation at position 328 within the CH2 domain, (L328F) and was encoded by a codon-optimised cDNA (SEQ ID NO: 149).

The mAb TNP1x24 comprised modified IgG1 heavy chain amino acid sequence (SEQ ID NO: 150) containing a point mutation at position 267 within the CH2 domain, (S267E) and was encoded by a codon-optimised cDNA (SEQ ID NO: 151).

The mAb TNP1x25 comprised modified IgG1 heavy chain amino acid sequence (SEQ ID NO: 152) containing a point mutation at position 267 within the CH2 domain, (S267E) and additionally a point mutation at position 328 within CH2 domain (L328F) and was encoded by a codon-optimised cDNA (SEQ ID NO: 153).

Mutations were introduced into the cDNA sequence encoding the H chain using standard molecular biology techniques including the synthesis of polynucleotide optimised for expression human cell expression.

### Expression and Production of IgG Antibodies by Expi293 Cells

The IgG antibodies were produced in Expi293 human embryonic kidney cells as described previously (Wines et al., 2016 supra). Briefly, Expi293 cells were maintained in Expi293 Expression Medium (Gibco, Waltham, MA, United States of America) for both cell growth and protein production. Cells were transfected simultaneously with the IgG heavy chain plasmid (15 μg) and light chain plasmid (15 μg) diluted in Opti-MEM I Reduced-Serum Medium (Gibco) using the Expifectamine transfection kit (Life Technologies) then cultured for four days. Culture supernatants were clarified by centrifugation and filtered through a 0.2 μm filter after which the IgGs were purified by affinity chromatography using a Hi-Trap HP Protein A column (GE Healthcare Life Sciences, Marlborough, MA, United States of America) and eluted with 0.1 M citric acid, pH 3.5, followed by neutralisation with 1M Tris-HCl, pH 9.0 and dialysation against phosphate buffered saline (PBS pH 7.5). Aggregates were removed by subsequent gel filtration on a Superose 6 10/300GL column (GE Healthcare Life Sciences) equilibrated in PBS and monomeric IgG peak fractions collected and used in subsequent analyses.

Evaluation of Antigen Binding Activity by Monoclonal Anti-TNP mAbs

The antigen binding activity of all anti-TNP antibody preparations was evaluated by ELISA for antigen binding activity by binding to TNP haptenylated BSA (TNP-BSA) as previously described in Wines et al., 2016 supra.

ELISA Immunoassay for Detection of Complement Fixation by mAbs

The wells of 96-well flat-bottom MaxiSorp Nunc plates (Thermo Fisher Scientific) were coated overnight at 4 °C with 50 μl of a solution of 20 μg/ml TNP-BSA in PBS. The following morning, plates were washed with a solution of PBS-Tween 0.05% (v/v) then treated for 2 hours at 37 °C with a solution of 1% (w/v) casein in PBS or alternatively the plates were treated with the 1% casein solution overnight at 4 °C The plates were washed with a solution of PBS-Tween 0.05% (v/v) and then 50 μL of serial dilutions of anti-TNP mAbs, commencing at 4 μg/ml, were added to the TNP BSA coated wells and incubated for 2 hours at RT. To measure C1q fixation, 10 μg/ml purified human C1q (Merck Millipore) was added to each well and incubated for 30 minutes at RT, washed with PBS-Tween 0.05% (v/v) then 50 μL of a 1/2000 dilution of rabbit anti-C1q IgG antibody (Kurtovic L et al., BMC Med 17:45 2019) was added to the wells and incubated for 1 hour at RT. Following the incubation wells were washed in PBS-Tween 0.05% (v/v) and incubated with 50 μL of 1/4000 dilution goat anti-rabbit IgG conjugated to HRP (Merck Millipore) for 1 hour at RT followed by washing in PBS-Tween 0.05% (v/v) then 100 μL of TMB substrate (Life Technologies) was added to each well for 8 minutes at RT. Reactivity was stopped using 1M sulfuric acid and absorbance was measured at OD450 nm. Test mAbs, antibodies and C1q reagents were prepared in solution of 0.1% (w/v) casein in PBS, and plates washed three times between each step using 0.05% (v/v) Tween20 in PBS. Samples were tested in duplicate and background reactivity was determined using negative control wells from which antibodies were omitted.

### Example 17 Anti-CD20 IgG Antibodies Comprising Mutation and Activity Analysis

Characteristics of the antibodies and any mutations and their SEQ IDs are detailed in Table 5.

### Production of Anti-CD 20 Human IgG and Mutant Anti-CD20 Human IgG Plasmid Constructs

Several CD20 specific mAbs, based rituximab variable domains and comprising two heavy chains (H2) and two light chains (L2) and thus the H2L2 format of a typical antibody, were produced and comprised unmodified H chains or comprised H chains which contained one or more mutations.

### Synthesis of Unmodified and Mutated Heavy Chains

Antibody expression vectors were generated by standard methods that are known to those skilled in the art. Briefly, antibody expression vectors consisted of a synthetic polynucleotide sequence, encoding the antibody heavy chain or light chain, appropriately placed within plasmids such as, for example, pcDNA3 and pcDNA3.4 (Thermo Fisher Scientific).

(a) Anti CD20 mAbs Comprising Unmodified, Wild Type (WT) Heavy (H) Chains.

The mAbs comprising unmodified H chains are as follows: The rituximab-IgG1-WT mAb used in this example comprised a CD20-specific heavy chain polypeptide (as described at https://go.drugbank.com/drugs/DB00073) (SEQ ID NO: 28) comprising in the order N-terminus to C-terminus, the CD20-specific VH domain of rituximab fused to CH1-hinge-CH2-CH3 domains of human IgG1 H chain, and is encoded by the codon-optimised DNA with the sequence shown as SEQ ID NO: 29.

The rituximab-IgG2-WT mAb used in this example comprised a CD20-specific heavy chain polypeptide (as described at https://go.drugbank.com/drugs/DB00073) (SEQ ID NO: 154) comprising in the order N-terminus to C-terminus, the CD20-specific VH domain of rituximab fused to CH1-hinge-CH2-CH3 domains of an unmodified human IgG2 heavy chain, and is encoded by the codon-optimised DNA with the sequence shown as SEQ ID NO: 155.

Similarly, the rituximab light chain used herein was as previously described at https://go.drugbank.com/drugs/DB00073 the polypeptide comprising the CD20-specific VL domain of rituximab fused to human kappa constant domain, (SEQ ID NO: 30) and is encoded by the codon-optimised DNA with the sequence shown as SEQ ID NO: 31.

(b) Anti-CD20, Rituximab, mAbs Comprising Modified H Chains

The CD20-specific mAbs, based on rituximab VL and VH variable domains and comprising IgG2 H chains were mutated to alter amino acid sequence of the H chains such that mutations were introduced into only the lower hinge or only the CH3 domain or were combined to generate mutated H chains comprising mutations in both the lower hinge and the CH3 domain of the heavy chain (Table 5).

The mAb RIT-IgG2-HF (SEQ ID NO: 156) comprised IgG2 heavy chains wherein histidine (H) position 429 (Eu numbering) within the CH3 domain was replaced with phenylalanine (F) and was encoded by the codon optimised cDNA (SEQ ID NO: 157). As outlined above the H429F mutation facilitates IgG hexamerisation.

Thus mAb RIT-IgG2-FLGG (SEQ ID NO: 158) comprised IgG2 heavy chains wherein the amino acid sequence, PVA (amino acid residues 233-236 Eu numbering as shown in FIG. 56 and Table 9) of the IgG2 lower hinge was replaced with the amino acid sequence, EFLG (amino acid residues 233-236 Eu numbering as shown in FIG. 56 and Table 9) from the lower hinge of IgG4 and was encoded by the codon optimised cDNA (SEQ ID NO: 159).

The mAb RIT-IgG2-FLGG-HF (SEQ ID NO: 160) comprised IgG2 heavy chains wherein the amino acid sequence, PVA (amino acid residues 233-236 Eu numbering as shown in FIG. 56 and Table 9) of the IgG2 lower hinge was replaced with the amino acid sequence EFLG (amino acid residues 233-236 Eu numbering as shown in FIG. 56 and Table 9) and additionally histidine (H) at position 429 (Eu numbering) within the CH3 domain was replaced with phenylalanine (F) and was encoded by the codon optimised cDNA (SEQ ID NO: 161). As outlined above the H429F mutation facilitates IgG hexamerisation.

The mAb RIT-IgG2-FLGG EG (SEQ ID NO: 162) comprised IgG2 heavy chains wherein the amino acid sequence, PVA (amino acid residues 233-236 Eu numbering as shown in FIG. 56 and Table 9) of the IgG2 lower hinge was replaced with the amino acid sequence EFLG and additionally glutamic acid (E) at position 430 within the CH3 domain was replaced with glycine (G) and was encoded by the codon optimised cDNA (SEQ ID NO: 163). The E430G mutation is known to facilitate IgG hexamerisation.

A series of mAbs were produced by replacement of PVA sequence (PVA, amino acid residues 233-236 Eu numbering as shown in FIG. 56 and Table 9) of the IgG2 lower hinge with the sequence, ELLG (amino acid residues 233-236 Eu numbering as shown in FIG. 56 and Table 9) from the lower hinge sequence of human IgG1 or were combined to generate mutated H chains comprising mutations in both the lower hinge and the CH3 domain of the heavy chain (Table 5). Thus: The mAb RIT-IgG2-LLGG (SEQ ID NO: 164) comprised IgG2 heavy chains wherein the amino acid sequence, PVA (amino acid residues 233-236 Eu numbering as shown in FIG. 56 and Table 9) of the IgG2 lower hinge was replaced with the amino acid sequence ELLG (amino acid residues 233-236 Eu numbering as shown in FIG. 56 and Table 9) from the lower hinge of IgG1 and was encoded by the codon optimised cDNA (SEQ ID NO: 165).

The mAb RIT-IgG2-LLGG-HF (SEQ ID NO: 166) comprised IgG2 heavy chains wherein the lower hinge sequence, PVA (amino acid residues 233-236 Eu numbering as shown in FIG. 56 and Table 9) of the IgG2 lower hinge was replaced with the amino acid sequence ELLG (amino acid residues 233-236 Eu numbering as shown in FIG. 56 and Table 9) from the IgG1 lower hinge and additionally histidine (H) at position 429 within the CH3 domain was replaced with phenylalanine (F) and was encoded by the codon optimised cDNA (SEQ ID NO: 167). As outlined above the H429F mutation facilitates IgG hexamerisation.

The mAb RIT-IgG2-LLGG-EG (SEQ ID NO: 168) comprised IgG2 heavy chains wherein the lower hinge sequence (PVA) was replaced with the amino acid sequence ELLG and additionally glutamic acid (H) position 430 within the CH3 domain was replaced with glycine (G) and was encoded by the codon optimised cDNA (SEQ ID NO: 169). The E430G mutation is known to facilitate IgG hexamerisation.

### Expression of Antibody Constructs

Expression of the antibodies was conducted using transient transfection of Expi293F cells (Thermo Fisher Scientific) with the relevant cDNA constructs. Expi293F cells were cultured in antibiotic-free Expi293 Expression Medium (Life Technologies) and, 24 hours prior to transfection cells, were split to a concentration of 2×10⁶ viable cells/ml. On the day of transfection, 7.5×10⁷viable log phase cells were centrifuged and resuspended in 25 ml of pre-warmed, antibiotic-free Expi293 Expression Medium and maintained at 37 °C until transfection. Transfection of the cells was then performed at RT using the Life Technologies Expifectamine Transfection Kit as follows. Eighty microlitres of ExpiFectamine 293 reagent was diluted with 1.5 ml Opti-MEM-I Reduced Serum Medium (Gibco) and incubated for 5 minutes at RT. Thirty μg of DNA (15 μg of H chain DNA and 15 μg of L chain DNA) was diluted in 1.5 ml of Opti-MEM-I reduced serum medium then added to the diluted ExpiFectamine reagent, incubated for 20-30 minutes at RT and then added dropwise to the Expi293F cell suspension which was then cultured at 37 °C for 16 to 18 hours at which time 150 μL of the manufacturer's Enhancer 1 and 1.5 ml of Enhancer 2 were added and the cells cultured for a further four days at 37 °C

Cell cultures were harvested and centrifuged at 2500 rpm for 20-30 minutes, and the supernatant filtered with a 0.2 μm high flow filter (Sartorius AG, G6ttingen, Germany) prior to purification. The presence of expected antibody in the supernatant was confirmed by SDS-PAGE.

Protein A Affinity Purification of mAbs

The mAbs were purified from the supernatant of the transfected Expi293F cells by Protein A affinity chromatography. Briefly, Hi-trap™ Protein A high-performance columns (GE Healthcare Life Sciences) were washed and equilibrated in binding buffer (20 mM NaH₂PO₄ pH 7.0) and the cell culture supernatant was loaded, the columns washed with binding buffer to baseline OD280 nm and bound antibody eluted with 0.1M sodium citrate tribasic dihydrate (pH 3.5) and 1 ml fractions collected and neutralised immediately with 1M Tris-HCl pH 9.0 and fractions containing the antibody, pooled.

Size Exclusion Chromatography (SEC) of mAbs

Following Protein A affinity purification, size exclusion chromatography (SEC) was used to further purify and characterise the antibodies. The Protein A purified antibodies were concentrated to an OD280 nm of 6-8 using a 30 kDa molecular weight cut-off centrifuge concentrator device (Merck-Millipore). Superose 6 Increase 10/300GL columns (GE Healthcare Life Sciences) were equilibrated in PBS pH 7.2, then the concentrated Protein A affinity purified antibody was loaded and separated at a flow rate of 0.5 ml/min in PBS and 0.5 ml fractions collected. For some mAbs, SEC was performed at pH 5.0, thus the concentrated Protein A purified mAb was dialysed overnight against buffer (100 mM sodium citrate, 100 mM NaCl, pH 5.0), then applied to a Superose 6 Increase 10/300 column pre-equilibrated in the same buffer. The dialysed antibody was applied to the Superose column SEC at a flow rate of 0.5 ml/min and 0.5 ml fractions collected from the column.

### Evaluation of Antigen Binding by Monoclonal Antibodies

Purified antibodies were tested for antigen binding prior to functional analysis. The mAbs recognising CD20 were tested for antigen recognition by flow cytometry as previously described in Trist et al., 2014 supra. The anti-CD20 mAbs based on rituximab were tested for binding on Ramos lymphoma cells expressing CD20. Briefly, mAbs were titrated by serial two-fold dilution in 25 μl of FACS buffer (PBS with 0.5% (w/v) BSA or Lebowitz-15 (L-15) medium containing 0.5% (w/v) BSA and lacking phenol red). Twenty-five microlitres of target cells at the concentration of 5×10⁶/ml were then added to the titrated mAb and incubated for 30 minutes on ice. Cells were then washed twice in FACS buffer, resuspended with 50 μL of F(ab)′₂ fragments of anti-human IgG-Fc conjugated to FITC and incubated for 30 minutes on ice, washed twice in cold FACS buffer and then resuspended in 50 μl of 2% paraformaldehyde/PBS and incubated for 30 minutes on ice, then washed once in FACS buffer and re-suspended in 200 μl FACS buffer. The cells were analysed by flow cytometry using a BD FACSCanto™ II flow cytometer (Becton Dickenson).

Evaluation of Complement Dependent Lysis of Cells Mediated by mAbs

CDC was measured by flow cytometry as described (Wines et al., Front Immunol 13:889372, 2022) using the Zombie Green Fixable Viability kit (BioLegend) following opsonisation of target cells with mAb.

Briefly, using 96-well plates the mAbs were serially two-fold diluted, starting at the initial concentration indicated in the figures, in 25 μl of FACS buffer (PBS/BSA (PBS containing 0.5% (w/v) BSA or Lebowitz-15 (L-15) medium containing 0.5% (w/v) BSA and lacking phenol red). Thus, for each WT mAb or mutated mAb, 25 μl of target cells, at a concentration of 5×10⁶/ml in 25 μl of FACS buffer were then added to the titrated mAbs and incubated for 30 minutes on ice. Cells were then washed twice in FACS buffer and resuspended in 50 μl of complement (normal human serum (NHS) diluted 1:3 in buffer immediately before use), and incubated for 30-45 minutes at 37 °C Following the incubation, the cells were washed twice in cold FACS buffer without BSA then resuspended in 50 μl Zombie Green (prepared in DMSO according to the manufacturer's instructions, BioLegend) diluted 1/500 in FACS buffer without BSA and incubated for a further 30 mins on ice, protected from light. These cells were then washed once in FACS buffer, resuspended in 50 μl of 2% paraformaldehyde in FACS buffer and incubated for 30 minutes on ice, then washed once in FACS buffer and re-suspended in 200 μl FACS buffer. The cells were analysed by flow cytometry using a BD FACSCanto™ II flow cytometer (Becton Dickinson). Dead cells were identified as those that stained with Zombie Green.

### Results and Discussion

### TNP mAb C1q Grouping

The anti-TNP antibodies comprising mutated heavy chains derived from the human IgG2 subclass or the human IgG1 subclass were evaluated for their capacity to bind C1q and the binding compared to that of anti-TNP antibodies comprising wild-type human IgG1 subclass heavy chain (TNP-IgG1-WT) or comprising wild-type human IgG2 subclass heavy chain TNP-IgG2-WT (FIGS. 57-59).

Group 1 Greatest C1_{q} Binding Relative to C1_{q} Binding by Human IgG2 and by Human IgG1

The mAbs showing greatest C1q binding relative to wild-type IgG2, TNP-IgG2-WT were TNP2x5, TNP2x4, (FIG. 57) TNP1x24 (FIG. 59) and the level of C1q binding was equivalent to the mAb TNP1x5 (FIG. 58B) wherein the heavy chains contain the E430G mutation, a modification in CH3 known to improve C1q binding by hexamerisation of IgG1. The mutated heavy chains of the mAbs TNP2x5 and TNP2x4 were based on human IgG2, a subclass regarded as having poor binding of C1q. The mutations used in these mAbs were the replacement of the IgG2 lower hinge sequence PVA. In mAb TNP2x5 the lower hinge amino acids EFLG were derived from human IgG4 also a subclass regarded as binding C1q poorly. Surprisingly this mutated mAb exhibited potent C1q binding. In the mAb TNP2x4 the lower hinge amino acid ELLG were derived from human IgG1 and surprisingly this small substitution of amino acids conferred profound C1q binding. Is also noteworthy that these mAbs also showed the greatest increase in C1q binding relative to wild-type human IgG1, TNP-IgG1-WT, which was especially surprising for the mAbs formatted on the IgG2 backbone. Thus it is clear that the choice of amino acids within the lower hinge in the context of IgG2 backbones is important for improved C1q binding which also constitutes the first step in the activation of the classical complement cascade.

The mAb TNP1x24 (FIG. 59) which also showed profound increase in C1q binding was formatted on an IgG1 backbone and contained the S267E mutation in the CH2 domain and which is known to improve C1q binding.

Group 2 Substantial Increase in C1_{q} Binding Relative to C1_{q} Binding by Human IgG2 and by Human IgG1

Second group of mAbs, TNP1x9, TNP1x10 (FIG. 58B) and TNP1x25 (FIG. 59) were all formatted on human IgG1 backbone and showed substantial increase in C1q binding compared to C1q binding by the TNP-IgG2-WT mAb. These mAbs contained mutations in the constant domains. In TNP1x9 the CH3 H435A mutation which is outside the canonical C1q binding site of IgG which is located in CH2 resulted in the, TNP1x10 mAb comprising a mutation in the CH1 domain wherein the S131C mutation introduces the cysteine at position 131 which mimics a site of light chain disulphide bonding in human IgG2, IgG3 and IgG4 which is absent in human IgG1. This mutation, on the IgG1 backbone provides a second site for light chain disulphide bonding in addition to the usual cysteine of the IgG1 upper hinge. This mutation surprisingly enhanced C1q binding. In the mAb TNP1x25, the heavy chain contained two point mutations, S267E and L328F, in the CH2 domain and exhibited improved C1q binding relative to both human IgG2 and human IgG1 (FIG. 59). Interestingly mAb TNP1x24 (FIG. 59) which contains only the S267E mutation showed greater C1q binding suggesting that the L328F mutation may impair C1q binding to some degree.

### Group 3 Showed Increased C1_{q} Binding Relative to Human IgG2 but Substantially Equivalent Binding Relative to Human IgG1

The third group of antibodies TNP1x32 (FIG. 58A) TNP1x14 (FIG. 58B) were formatted on a human IgG1 backbone and TNP2x15 (FIG. 57) was formatted on a human IgG2 backbone. For the mAbs TNP1x32 and TNP1x14 enhanced binding of C1q was apparent relative to C1q binding by TNP-IgG2-WT. However, C1q binding of these mutated mAbs was equivalent to human TNP-IgG1-WT, the H chains of which were used as the backbone of these mutated mAbs. The TNP1x32 heavy chain was derived from human IgG1 but the upper hinge sequence was exchanged for sequence derived from the upper hinge of IgG2 which results in the placement of two cysteine residues into the upper hinge of this mAb rather than the single cysteine residue found in the upper hinge of IgG1 which is used for light chain disulphide bonding. Thus, the replacement of IgG upper hinge sequence with the IgG2 upper hinge sequence provides an additional site for light chain disulphide bonding or for inter-heavy chain disulphide bonding. The mAb TNP1x14 is formatted on IgG1 heavy chain backbone but which has two mutations, S131C in CH1 and substitution of the upper hinge amino acids with amino acids derived from the upper hinge of human IgG4. The replacement of the IgG1 upper hinge alters amino acid sequence which also results in the replacement of a cysteine residue normally used for light chain disulphide bonding in human IgG1, however the additional mutation S131C in the CH1 domain provides a new site for disulphide bonding of light chain (mimicking the light chain disulphide bonding site of IgG2, IgG3, and IgG4). Interestingly the Group 1 mAb TNP1x10 (FIG. 58B) which contains only the S131C mutation showed greater C1q binding than TNP1x14 suggesting that the nature of the upper hinge and the nature of light chain disulphide bonding is important in C1q binding.

The TNP2x15 (FIG. 57) mAb mutated IgG2 H chains were engineered wherein the lower hinge PVA sequence was replaced with EFEG i.e. PVA(233-236)EFEG mutation resulted in enhanced C1q binding relative TNP-IgG2-WT but this binding was equivalent to that seen by the wild-type mAb TNP-IgG1-WT. Interestingly MAb TNP2x15 differs by a single amino acid (E at position 235) from TNP2x5 (L at position 235) and whose binding of C1q was amongst the highest observed (Group 1, FIG. 57). Thus, point mutation within the lower hinge can affect the level of C1q binding.

### Group 4 Substantially Equivalent C1q Binding Relative to Human IgG2 but Impaired C1q Binding Relative to Human IgG1

These mAbs, TNP1x23 (FIG. 59) and TNP2x17 (FIG. 57) both show poor C1q binding which was at a level equivalent to the poor C1q binding observed for TNP-IgG2-WT. TNP1x23 is formatted on a human IgG1 heavy chain backbone which also carries the L328F mutation in its CH2 domain and results in a profound impairment in C1q binding compared to the C1q binding of wild-type human IgG1 which indicates position 328 plays a key role in C1q binding. Indeed, combining the L238F mutation with the S267E mutation in the IgG1 heavy chains of the mAb TNP1x25 diminished C1q binding compared to the profoundly enhanced C1q binding by mAb TNP1x24 which comprised mutated IgG1 heavy chains containing only the S267E mutation.

The mAb TNP2x17 (FIG. 57) is formatted on an IgG2 heavy chain backbone but amino acids in the upper hinge of IgG2 were replaced with amino acids from the upper hinge of IgG1. The C1q binding was poor and equivalent to that observed by wild-type human IgG2, indicating that the mutation did not affect the already poor C1q binding of the human IgG2 heavy chain backbone in this mutant.

### Group 5 Substantially Impaired C1q Binding Relative to Both Human IgG2 and Human IgG1

The mAbs TNP1x 15, TNP1x 12 (FIG. 58B) and TNP1x 16 (FIG. 59) were each formatted on human IgG1 heavy chains but following mutation in the CH2 domains of the heavy chains all failed to demonstrate a detectable binding of C1q. The mutated IgG1 heavy chains of the mAbs TNP1x15 and TNP1x12 contain glyco-shift mutations designed to move the normal, wild type, site of N-linked glycosylation which is found in all human IgG subclasses at asparagine (N) at position 297 (N297). In the mAb TNP1x15 the mutation QYN295-297NST in the heavy chain places the asparagine at position 295 and also removes the wild-type IgG1 glycosylation site by replacing the N297 with threonine. In the mAb TNP1x12, the mutation YNS296-298NSS in the heavy chain places the asparagine at position 296 and also removes the wild-type IgG1 glycosylation site by replacing the N297 with serine. The glyco-shift mutations reduce C1q binding to levels lower than that of the poor C1q binding of TNP-IgG2-WT.

In the mAb TNP1x 16 (FIG. 59) the mutated IgG1 heavy chains contain the ED(269,270)AA mutation which are adjacent point mutations in the CH2 domain. The combination of these point mutations reduces C1q binding to levels lower than that of the poor C1q binding by TNP-IgG2-WT.

### CD38 Specific mAb C1q/C1 Grouping.

The anti-CD38 antibodies comprising mutated heavy chains derived from the human IgG2 subclass or the human IgG1 subclass, with variable domains derived from isatuximab, were evaluated for their capacity to bind C1q, as either C1q (FIG. 85) or as C1 (FIGS. 71, 78), and the binding compared to that of anti-CD38 antibodies comprising wild-type human IgG1 subclass heavy chain (Isa-IgG1-WT) or comprising wild-type human IgG2 subclass heavy chain (Isa-IgG2-WT) (FIGS. 71, 78, 85).

Group 2 mAbs with Isatuximab specificity included Isa-IgG2-EFLGG with the lower hinge mutation PVA(233-236)EFLG, Isa-IgG2-M1 with the mutations C131S-C219S-PVA(233-236) EFLG, and Isa-IgG2-M2 comprising the H-chain mutations C131S-C220S-PVA(233-236)EFLG. The C1q/C1 binding activity of a mAb with an IgG2 backbone can be substantially increased by the modification of the lower hinge, and cysteines C131 and C219 or C220 (FIGS. 71, 78).

Group 3 mAbs with Isatuximab specificity included Isa-IgG2-M3 with the mutations C131S-ERKCCVE(216-225)EPKSCDKTHT-PVA(233-236)EFLG. The C1q/C1 binding activity of a mAb with an IgG2 backbone can be increased relative to IgG2-WT by the modification of the lower hinge, and cysteine C131 and the substitution of the upper hinge sequence with that from IgG1 (FIGS. 71, 78).

Group 4 mAbs have equivalent C1q binding relative to human IgG2 but impaired C1q binding relative to human IgG1. Group 4 mAbs with Isatuximab specificity comprised Isa-IgG1-K274Q with the H-chain mutation K274Q in the CH2 domain (FIG. 85) where a deficiency in C1q binding results from the K274Q mutation. This inhibition of C1q binding is suggested to underlie the diminished CDC activity of Isa-IgG1-K274Q compared to Isa-IgG1-WT (FIG. 84). Isa-IgG1-LALA, with the lower hinge mutation L234A, L235A (FIG. 85), also exhibits a deficiency in C1q binding resulting from the L234A, L235A mutation. This inhibition of C1q binding is consistent with the diminished CDC activity of Isa-IgG1-LALA compared to Isa-IgG1-WT (FIG. 84). The placement of Isa-IgG2-C131S-C219S with the disulfide mutations C131S, C219S in the IgG2 H-chain within group 4 is predicted from the equivalent CDC activity of this mAb with Isa-IgG2-WT (FIG. 70C, D).

Group 5 with substantially impaired C1q binding and Isatuximab specificity is predicted to describe Isa-IgG2-Q274K with the H-chain mutation Q274K in the CH2 domain (FIG. 85). This deficiency in C1q binding resulting from the Q274K mutation inhibiting C1q binding is wholly consistent with the diminished CDC activity of Isa-IgG2-Q274K-H429F compared to Isa-IgG2-H429F (FIG. 84).

### Complement-Dependent Cytotoxicity Mediated by Rituximab Variants Comprising Mutated Heavy Chains Based on Human IgG2 Subclass

Complement dependent cytotoxicity (CDC) was determined for several mAbs targeting human CD20 antigen. The variable light chain domain and variable heavy chain domain of the mAbs were derived from rituximab an anti-CD20 mAb. The light chain was formatted as a human kappa chain (Rit-L) and heavy chains were formatted on a wild-type IgG2 subclass H chain backbone (Rit-IgG2-WT) or mutated IgG2 subclass heavy chain.

Prior to evaluation in CDC assays the mAbs were tested for their capacity to bind to CD20 (FIG. 60) and was compared to the binding by rituximab comprising wild-type IgG1 heavy chains (Rit-IgG1-WT). Binding was evaluated by flow cytometry on Ramos lymphoma cells expressing CD20. The data shows that the mAbs comprising mutated IgG2 H chains bound similarly to one another and to Rit-IgG2-WT which comprised the unmodified wild-type heavy chains (FIG. 60). In every case the level of binding of these IgG2-based mAbs was surprisingly and considerably lower than the binding of rituximab comprising wild-type heavy chains of the IgG1 subclass (Rit-IgG1-WT), indicating that the level of opsonisation of the CD20 target by the IgG2-based mAbs was considerably poorer than that by the IgG1 based mAb.

Notwithstanding poor opsonisation by the IgG2-based mAbs, the extent of CDC mediated by these mAbs was determined using human Daudi cells expressing CD20 and compared to CDC mediated by the Rit-IgG2-WT (FIG. 61).

As expected, Rit-IgG2-WT which comprised unmodified wild type IgG2 heavy chains failed to mediate detectable CDC. However, the replacement of the lower hinge sequence of IgG2 with amino acids from the lower hinge of IgG4 or IgG1 resulted in readily detectable increase in CDC (FIG. 61A). The mAb Rit-IgG2-FLGG which comprised mutated IgG2 heavy chains wherein IgG2 lower hinge sequence was replaced with the amino acids from the IgG4 lower hinge clearly mediated detectable CDC of Daudi cells. The CDC mediated by Rit-IgG2-FLGG was clearly greater than the absence of CDC observed with the Rit-IgG2-WT mAb even at concentrations as high as 40 μg/ml. (FIG. 61A). This is all-the-more remarkable since human IgG4 and human IgG2 are known to be poor mediators of CDC. The mAb Rit-IgG2-LLGG which comprised mutated IgG2 heavy chains wherein the PVA sequence of the IgG2 lower hinge was replaced with the amino acids from the IgG1 lower hinge also mediated detectable CDC of Daudi cells (FIG. 61A) This improved CDC is entirely consistent with the observed improvement in the binding of C1q in the ELISA assay (FIG. 57) wherein replacement of the lower hinge of IgG2 with lower hinge sequences from IgG4 or IgG1 (TNP2x5 and TNP2x4) resulted in enhanced C1q binding relative to IgG2 wild-type heavy chains (FIG. 57).

Further mutations were made and CDC of these anti-CD20 rituximab-based mAbs was undertaken (FIG. 61B). The Rit-IgG2-FLGG HF mAb IgG2 based heavy chains wherein the lower hinge PVA sequence of IgG2 was replaced with the lower hinge of IgG4 and additionally a point mutation H429F was made in CH3. The addition of the H429F mutation further enhanced CDC observed for the Rit-IgG-FLGG mAb that lacks the H429F mutation (FIG. 61A)

A second mAb Rit-IgG2-LLGG HF which comprised IgG2 based heavy chains wherein the lower hinge of IgG2 was replaced with the lower hinge of IgG1 and additionally a point mutation. The addition of the H429F mutation further enhanced the CDC observed for the Rit-IgG2-LLGG mAb.

Thus, it is clear that CDC may be conferred on IgG2 H chains following mutation of the lower hinge sequence can be further improved by additional mutation such as the H429F.

The Rit-IgG2-FLGG EG which comprised IgG2 based heavy chains wherein the lower hinge of IgG2 was replaced with the lower hinge of IgG4 and additionally a point mutation E430G was made in CH3. It should be noted that the point mutation E430G is known to enhance CDC through IgG hexamerisation. The addition of the E430G mutation further enhanced CDC observed for the Rit-IgG-FLGG mAb.

A second mAb Rit-IgG2-LLGG EG which comprised IgG2 based heavy chains wherein the lower hinge of IgG2 was replaced with the lower hinge of IgG1 and additionally a point mutation. The addition of the E430G mutation further enhanced CDC observed for the Rit-IgG2-LLGG mAb.

Thus, it is clear that CDC may be conferred on IgG2 H chains following mutation of the lower hinge sequence can be further improved by additional mutation such as the E430G.

The binding of C1q to an antibody may be affected in different ways. Altering the amino acids in the immunoglobulin heavy chain at the interaction interface between the immunoglobulin heavy chain and C1q may increase or decrease the affinity of immunoglobulin interaction with C1q. The interaction interface may include one or more sites within CH2 of interaction between the heavy chain and C1q. Alternatively, C1q binding may be increased or decreased by altering amino acids outside the C1q binding site, for example in the CH3 domain wherein those modifications alter the binding affinity of C1q by allosteric effects on the C1q binding interface in CH2. Alternatively, modifications outside the C1q binding interface for example in the CH3 domain or in the upper hinge or the CH1 domain change the physical behaviour of the immunoglobulin such that the modified antibody forms multimeric structures such as hexamers of immunoglobulin on the target to which the Ab is bound thereby providing a platform for more avid C1q binding with or without concomitant alteration in the affinity of C1q binding.

TABLE 5
Characteristics of the antibodies and any mutations

| | Constant region | | | | | | |
|---|---|---|---|---|---|---|---|
| | heavy | | | | | | |
| | chain | | | | | amino | Sequence ID # |
| Name | subclass | CH1 | Hinge | CH2 | CH3 | acid | nucleotide |
| TNP-IgG1-WT | 1 | WT | WT | WT | WT | 32 | 33 |
| TNP-IgG2-WT | 2 | WT | WT | WT | P445L | 34 | 35 |
| TNP-IgG4-WT | 4 | WT | WT | WT | WT | 114 | 115 |
| TNP-Light chain | NA | kappa constant light chain | | | | 36 | 37 |
| TNP2 × 5 | 2 | WT | WT | PVA(233-236)EFLG | WT | 116 | 117 |
| TNP2 × 4 | 2 | WT | WT | PVA(233-236)ELLG | WT | 118 | 119 |
| TNP2 × 15 | 2 | WT | WT | PVA(233-236)EFEG | *P445L | 120 | 121 |
| TNP2 × 17 | 2 | WT | RKCCVE(217-225)PKSCDKTHT | WT | *P445L | 122 | 123 |
| TNP1 × 32 | 1 | WT | EPKSCDKTHT(216-225)ERKCCVE | WT | WT | 124 | 125 |
| TNP1 × 3 | 1 | WT | WT | WT | H429F | 126 | 127 |
| TNP1 × 5 | 1 | WT | WT | WT | E430G | 128 | 129 |
| TNP1 × 1 | 1 | WT | WT | WT | H429Q | 130 | 131 |
| TNP1 × 2 | 1 | WT | WT | WT | H429E | 132 | 133 |
| TNP1 × 9 | 1 | WT | WT | WT | H435A | 134 | 135 |
| TNP1 × 10 | 1 | S131C | WT | WT | WT | 136 | 137 |
| TNP1 × 14 | 1 | S131C | EPKSCDKTHT(216-225)ESKYGPP | WT | WT | 138 | 139 |
| TNP1 × 15 | 1 | WT | WT | QYN(295-297)NST | WT | 140 | 141 |
| TNP1 × 12 | 1 | WT | WT | YNS(296-298)NSS | WT | 142 | 143 |
| TNP1 × 16 | 1 | WT | WT | ED(269, 270)AA | WT | 144 | 145 |
| TNP1 × 22 | 1 | WT | WT | WT | H429S | 146 | 147 |
| TNP1 × 23 | 1 | WT | WT | L328F | WT | 148 | 149 |
| TNP1 × 24 | 1 | WT | WT | S267E | WT | 150 | 151 |
| TNP1 × 25 | 1 | WT | WT | S267E, L328F | WT | 152 | 153 |
| Rit-IgG1-WT | 1 | WT | WT | WT | WT | 28 | 29 |
| Rit-IgG2-WT | 2 | WT | WT | WT | WT | 154 | 155 |
| Rituximab light chain | NA | kappa constant light chain | | | | 30 | 31 |
| (Rit-L) | | | | | | | |
| Rit-IgG2 HF | 2 | WT | WT | WT | H429F | 156 | 157 |
| Rit-IgG2 FLGG | 2 | WT | WT | PVA(233-236)EFLG | WT | 158 | 159 |
| Rit-IgG2 FLGG HF | 2 | WT | WT | PVA(233-236)EFLG | H429F | 160 | 161 |
| Rit-IgG2 FLGG EG | 2 | WT | WT | PVA(233-236)EFLG | E430G | 162 | 163 |
| Rit-IgG2 LLGG | 2 | WT | WT | PVA(233-236)ELLG | WT | 164 | 165 |
| Rit-IgG2 LLGG HF | 2 | WT | WT | PVA(233-236)ELLG | H429F | 166 | 167 |
| Rit-IgG2 LLGG EG | 2 | WT | WT | PVA(233-236)ELLG | E430G | 168 | 169 |
| Rit-IgG3-KVH | 3 | WT | WT | WT | N392K, | 170 | 171 |
| | | | | | M397V, | | |
| | | | | | R435H | | |
| Rit-IgG3-KVH HF | 3 | WT | WT | WT | N392K, | 172 | 173 |
| | | | | | M397V, | | |
| | | | | | R435H, | | |
| | | | | | H429F | | |
| Rit-IgG4 | 4 | WT | WT | WT | WT | 174 | 175 |
| Rit-IgG4 HF | 4 | WT | WT | WT | H429F | 176 | 177 |

*P445L is not a C1q binding modification
It should be noted that the lower hinge sequence of IgG2 can be depicted as PVA, P-VA, PV-A. As is widely known the dash “-” in the sequence string is simply introduced for the purposes of alignment of amino acids as for example the alignment of hinge sequences of the immunoglobulin subclasses.

TABLE 6
Mutation groupings

| | | Relative to | | | | | | | |
|---|---|---|---|---|---|---|---|---|---|
| C1q binding | | C1q IgG1 | | | | | LH/ | | |
| group | | binding | Antibody | Mutation | CH1 | UH* | CH2* | CH2 | CH3 |
| | Relative to | | | | | | | | |
| | IgG2 C1q | | | | | | | | |
| | binding | | | | | | | | |
| C1q Group | 6+ | 4+ | TNP2 × 5 | PVA(233- | | | X | | |
| 1 | | | | 236)EFLG | | | | | |
| | | | TNP2 × 4 | PVA(233- | | | X | | |
| | | | | 236)ELLG | | | | | |
| | | | TNP1 × 5 | E430G | | | | | X |
| | | | TNP1 × 24 | S267E | | | | X | |
| C1q Group | 4+ | 2+ | TNP1 × 9 | H435A | | | | | x |
| 2 | | | TNP1 × 10 | S131C | x | | | | |
| | | | TNP1 × 25 | S267E, L328F | | | | x | |
| | | | Isa-IgG2- | PVA(233- | | | x | | |
| | | | EFLGG | 236)EFLG | | | | | |
| | | | Isa-IgG2-M1 | C131S-C219S- | x | x | x | | |
| | | | | PVA(233-236) | | | | | |
| | | | | EFLG | | | | | |
| | | | Isa-IgG2-M2 | C131S-C220S- | x | x | x | | |
| | | | | PVA(233-236) | | | | | |
| | | | | EFLG | | | | | |
| C1q Group | 2+ | = | TNP1 × 32 | EPKSCDKTHT(216- | | x | | | |
| 3 | | | | 225)ERKCCVE | | | | | |
| | | | TNP1 × 14 | EPKSCDKTHT(216- | | x | | | |
| | | | | 225)ESKYGPP | | | | | |
| | | | TNP2 × 15 | PVA(233- | | | x | | |
| | | | | 236)EFEG | | | | | |
| | | | Isa-IgG2-M3 | C131S-ERKCCVE | x | x | x | | |
| | | | | (216-225) | | | | | |
| | | | | EPKSCDKTHT- | | | | | |
| | | | | PVA(233-236) | | | | | |
| | | | | EFLG | | | | | |
| C1q Group | = | 2− | TNP1 × 23 | L328F | | | | x | |
| 4 | | | TNP2 × 17 | RKCCVE(217- | | x | | | |
| | | | | 225)PKSCDKTHT | | | | | |
| | | | Isa-IgG1- | K274Q | | | x | | |
| | | | K274Q^{#} | | | | | | |
| | | | Isa-IgG1- | LALA | | | x | | |
| | | | LALA^{##} | | | | | | |
| | | | Isa-IgG2- | C131S, C219S | x | x | | | |
| | | | C131S- | | | | | | |
| | | | C219S^{###} | | | | | | |
| C1q Group | 2− | 4− | TNP1 × 15 | QYN(295-297)NST | | | | x | |
| 5 | No | (NDB) | TNP1 × 12 | YNS(296-298)NSS | | | | x | |
| | detectable | | TNP1 × 16 | ED(269, 270)AA | | | | x | |
| | binding | | Isa-IgG2- | Q274K | | | x | | |
| | (NDB) | | Q274K^{####} | | | | | | |
| | Relative | | | | | | | | |
| | CDC to | | | | | | | | |
| | IgG2** | | | | | | | | |
| CDC Group | CDC 4+ | | Rit-IgG2 | PVA(233- | | | x | | x |
| 1 | | | FLGG EG | 236)EFLG, E430G | | | | | |
| | | | Rit-IgG2 | PVA(233- | | | x | | x |
| | | | LLGG EG | 236)ELLG, E430G | | | | | |
| CDC Group | CDC 2+ | | | | | | | | |
| 2 | | | | | | | | | |
| CDC Group | CDC 1+ | | Rit-IgG2 | PVA(233- | | | x | | |
| 3 | | | FLGG | 236)ELLG | | | | | |
| | | | Rit-IgG2 | PVA(233- | | | x | | |
| | | | LLGG | 236)ELLG | | | | | |

*LH = lower hinge; UH = upper hinge
**IgG2 has no detectable killing (NDCDC)
^{#}C1q binding activity predicted from CDC activity, FIG. 84.
^{##}C1q activity predicted from CDC activity, FIG. 84 and C1q binding assay FIG. 85.
^{###}C1q activity predicted from CDC activity, FIG. 84.
^{####}C1q binding activity predicted from CDC activity of Isa-IgG2-Q274K-H429F compared to Isa-IgG2-H429F, FIG. 84; C1q binding assay FIG. 85.

### Example 18 Activity Analysis of Anti-CD20 IgG2 mAbs with Lower Hinge Mutations in Addition to the H429F Stellabody Mutation

Human IgG exists as four subclasses (IgG1, IgG2, IgG3, and IgG4) that differ in structure and function. Many effector functions of IgG are elicited by immune complexes of antibodies with the Fc portions of the complexes interacting avidly with FcγRs (Lux et al., J Immunol 190:4315-4323, 2013) or with the C1 complex of the classical complement pathway via C1q (Reid, K. B. M, Front Immunol 9:764, 2018). Although sharing over 90% similarity in the amino acid sequence of their constant regions, each subclass exhibits a vastly different capability to elicit effector responses. Notably, the greatest sequence variation that occurs between the IgG subclasses exists within the upper hinge and lower hinge/upper CH2 regions that participate in, or influence, key FcγR and C1q interactions. These differences in the upper and lower hinge may underlie, or contribute to, the different abilities of each subclass to elicit potent effector responses and could therefore be used to inform the design of new therapeutic mAbs with unique effector function potencies and profiles.

IgG2 interacts with the H131 allotype of FcγRIIa with an affinity of 4.5×10⁵ M⁻¹, but with all other FcγRs at much lower, e.g. FcγRIIb (K_{A}=0.2×10⁵ M⁻¹), or undetectable affinities (Bruhns et al., Blood 113:3716-3725, 2009. IgG2 immune complexes interact poorly with C1q, but effectively with C1, where the protease subcomponents, C1r2C1s2, stabilise the complex between C1 and IgG2. Thus antigens, such as gram-positive bacteria, which can present a dense array of opsonising IgG2 can efficiently activate the complement pathway via C1. Furthermore, mutation of IgG2, to enhance Fc: Fc interactions and the formation of hexamers enhances the interaction of C1q/C1 and subsequent activation of complement (Zwarthoff et al., Proc Natl Acad Sci USA 118, 2021).

The most sequence diverse areas of immunoglobulins outside the variable domains are the hinge sequences as seen in the alignments of amino acid sequence of the constant regions and hinges of the Ig heavy chains (FIG. 3). It should also be noted the CH2 domain sequence also incorporates sequences that are considered structurally as part of the hinge region of the IgG protein, and which is referred to in the field and herein as the lower hinge e.g. APELLGG of IgG1 and IgG3, APEFLGG of IgG4 and APPV-AG of IgG2. A more detailed alignment of the hinges of the human IgG subclasses is shown for clarity (Table 7, 8 and 9) and show the hinges comprise an upper hinge, core hinge and lower hinge.

The human IgG2 heavy chain is unique as it is the only IgG subclass wherein the upper hinge is constrained as a result of inter- and intra-heavy chain disulphide bonds and the light chain can attach in one of three positions—cysteine 131 in the CH1 domain, or cysteine 219 and cysteine 220 in the upper hinge (Table 7 and FIG. 65).

TABLE 7
Sequence alignment for human IgG upper hinge regions
arranged according to IMGT ®, the
international ImMunoGeneTics information system ®
(https://www.imgt.org/IMGTScientificChart/
Numbering/Hu_IGHGnber.html). Dashes are
introduced into the sequences to account for
differing hinge length and homology. Sequence homology is
indicated by bold text. Amino acid residues are annotated
according to Eu numbering. The IgG3 sequence provided
is that of the H1 exon. upper hinge

| Eu number | 216 | 217 | 218 | | | 219 | 220 | 221 | 222 | 223 | 224 | 225 |
|---|---|---|---|---|---|---|---|---|---|---|---|---|
| IgG1 HC (J00228) | E | P | K | — | — | S | C | D | K | T | H | T |
| IgG2 HC (J00230) | E | R | K | — | — | C | C | — | V | — | E | — |
| IgG3 HC (M12958) | E | L | K | T | P | L | G | D | T | T | H | T |
| H1 | | | | | | | | | | | | |
| IgG4 HC (K01316) | E | S | K | Y | G | — | — | — | — | — | P | P |

TABLE 8
Sequence alignment for human IgG upper hinge regions arranged according to
IMGT ®, the international ImMunoGeneTics information system ®
(https://www.imgt.org/IMGTScientificChart/Numbering/Hu_IGHGnber.html)
Sequence homology is indicated by bold text. Amino acid residues
are annotated according to Eu numbering. Depending on
the allelic variant the upper and core hinge region of IgG3
may be encoded by up to four exons, H1, H2, H3, and H4.
*The IgG3 sequence 226-228 provided is that of the H1 exon
and the amino acid residues C229 and C230 are assigned by
to exon H4, but each hinge exon includes a complete CPRCP sequence.
core hinge

| Eu number | 226 | 227 | 228 | 229 | 230 |
|---|---|---|---|---|---|
| IgG1 HC (J00228) | C | P | P | C | P |
| IgG2 HC (J00230) | C | P | P | C | P |
| IgG3 HC (M12958) | C | P | R | C* | P* |
| IgG4 HC (K01316) | C | P | S | C | P |

TABLE 9
Sequence alignment for human IgG lower hinge regions arranged according to
IMGT ®, the international ImMunoGeneTics information system ®
(https://www.imgt.org/IMGTScientificChart/Numbering/Hu_IGHGnber.html). The
lower hinge is encoded by the N-terminal residues of the CH2 exon. Sequence
homology is indicated by bold text and the dash indicates the human
IgG2 lower hinge is one amino acid residue shorter than those of IgG1, IgG3
or IgG4. Sequence homology is indicated by shading. Amino acid residues are
annotated according to Eu numbering.
lower hinge

| Eu number | 231 | 232 | 233 | 234 | 235 | 236 | 237 |
|---|---|---|---|---|---|---|---|
| IgG1 HC (J00228) | A | P | E | L | L | G | G |
| IgG2 HC (J00230) | A | P | P | V | — | A | G |
| IgG3 HC (M12958) | A | P | E | L | L | G | G |
| IgG4 HC (K01316) | A | P | E | F | L | G | G |

In line with the poor interaction of IgG2 with all but one FcγR and with C1q, IgG2 lacks several key residues in the lower hinge that are proposed to participate in, or influence, interactions with C1q and FcγRs (Table 9). Exchanging lower hinge residues (ELLG, amino acids 233-236; EU numbering) in an IgG1 to those found in IgG2 (PV-A) has previously been shown to abolish effector functions such as CDC and binding to FcγRI, highlighting the importance of this region in IgG1 interaction with FcγRs and C1q (Morgan et al., Immunol 86(2):319-325, 1995). This sequence exchange could also be described as exchanging lower hinge residues (ELLGG, amino acids 233-237; EU numbering) in an IgG1 to those found in IgG2 (PV-AG). This sequence exchange could also be described as an ELLGG→PV-AG exchange of sequences in the lower hinge region of these antibodies.

In this example, the lower hinge sequence of IgG2 (PV-AG) was replaced with either the corresponding lower hinge sequence of IgG4 (‘EFLGG’; amino acids 233-237), or with that of IgG1 (‘ELLGG’; amino acids 233-237). This same sequence exchange could also be described as exchanging lower hinge residues (PV-A, amino acids 233-236; EU numbering) in an IgG2 to those found in IgG4 (EFLG, amino acids 233-236; EU numbering).

The effect of these lower hinge modifications on the ability of anti-CD20 IgG2 mAbs induce target cell killing was assessed. Furthermore, the effect of the H429F substitution on the properties of these anti-CD20 IgG2 mAbs was also examined.

Additionally, the antigen binding capability of IgG2 was assessed relative to the other human IgG subclasses, all of which were directed against the cell surface antigen CD20 using the variable domain of the anti-CD20 mAb, rituximab. The construction of the mAbs and their sequence IDs are described below.

### Methods and Materials

### Antibodies and Antibody Constructs.

All anti-CD20 mAbs used in this example carried the V domain of rituximab, that comprised the light chain polypeptide with the amino acid sequence as shown in SEQ ID NO: 30 (comprising the CD20-specific VL domain of the rituximab mAb fused to human kappa constant domain) and is encoded by the codon-optimised DNA with the sequence shown as SEQ ID NO: 31.

The heavy chain sequences of the mAbs used in this example are described below. Modified mAb heavy chains were produced by the synthesis of synthetic cDNA sequences encoding the antibody heavy chain with the relevant mutations using standard molecular biology techniques. The amino acid modifications that were introduced into the rituximab-IgG1-WT and rituximab-IgG2-WT heavy chain are described below.

The RIT-IgG1-WT heavy chain polypeptide comprised, in the order of the N-terminus to the C-terminus, the CD20-specific VH domain of rituximab fused to the CH1-hinge-CH2-CH3 domains of human IgG1, as shown in SEQ ID NO: 28, and is encoded by the codon-optimised DNA with the sequence shown as SEQ ID NO: 29.

The RIT-IgG2-WT heavy chain polypeptide comprised in the order of the N-terminus to the C-terminus, the CD20-specific VH domain of rituximab fused to the CH1-hinge-CH2-CH3 domains of human IgG2, as shown in SEQ ID NO: 154 and is encoded by the codon-optimised DNA with the sequence shown as SEQ ID NO: 155.

The RIT-IgG3-WT heavy chain polypeptide comprised, in the order of the N-terminus to the C-terminus, the CD20-specific VH domain of rituximab fused to the CH1-hinge-CH2-CH3 domains of human IgG3, as shown in SEQ ID NO: 38, and is encoded by the codon-optimised DNA with the sequence shown as SEQ ID NO: 39.

The RIT-IgG4-WT heavy chain polypeptide comprised, in the order of the N-terminus to the C-terminus, the CD20-specific VH domain of rituximab fused to the CH1-hinge-CH2-CH3 domains of human IgG4, as shown in SEQ ID NO: 40, and is encoded by the codon-optimised DNA with the sequence shown as SEQ ID NO: 41.

The RIT-IgG1-H429F heavy chain polypeptide comprised, in the order of the N-terminus to the C-terminus, the CD20-specific VH domain of rituximab fused to the CH1-hinge-CH2-CH3 domains of human IgG1 with a substitution of histidine with phenylalanine at position 429 (H429F; Eu numbering) in the CH3 region, as shown in SEQ ID NO:222, and is encoded by the codon-optimised DNA with the sequence shown as SEQ ID NO:223.

The RIT-IgG2-H429F heavy chain polypeptide comprised, in the order of the N-terminus to the C-terminus, the CD20-specific VH domain of rituximab fused to the CH1-hinge-CH2-CH3 domains of human IgG2 with a substitution of histidine with phenylalanine at position 429 (H429F; Eu numbering) in the CH3 region, as shown in SEQ ID NO: 156, and is encoded by the codon-optimised DNA with the sequence shown as SEQ ID NO: 157.

The RIT-IgG2-EFLGG (a.k.a. RIT-IgG2-FLGG) heavy chain polypeptide comprised, in the order of the N-terminus to the C-terminus, the CD20-specific VH domain of rituximab fused to the CH1-hinge-CH2-CH3 domains of human IgG2 with the following mutation: the residues at position 233-237 of IgG2 (PV-AG) in the lower hinge were substituted with the amino acids (EFLGG) from the lower hinge of the human IgG4 subclass. The amino acid sequence is shown in SEQ ID NO: 158, and is encoded by the codon-optimised DNA with the sequence shown as SEQ ID NO: 159.

The RIT-IgG2-EFLGG-H429F (a.k.a. RIT-IgG2-FLGG-HF) heavy chain polypeptide comprised the CD20-specific heavy chain polypeptide of RIT-IgG2-EFLGG with a substitution of histidine with phenylalanine at position 429 in the CH3 region (H429F; Eu numbering). The amino acid sequence is shown in SEQ ID NO: 160 and is encoded by the codon-optimised DNA with the sequence shown as SEQ ID NO: 161.

The RIT-IgG2-ELLGG (a.k.a. RIT-IgG2-LLGG) heavy chain polypeptide comprised, in the order of the N-terminus to the C-terminus, the CD20-specific VH domain of rituximab fused to the CH1-hinge-CH2-CH3 domains of human IgG2 with the following mutation: the residues at position 233-237 of IgG2 (PV-AG) in the lower hinge were substituted with the amino acids (ELLGG) from the lower hinge of the human IgG1 subclass. The amino acid sequence is shown in SEQ ID NO: 164 and is encoded by the codon-optimised DNA with the sequence shown as SEQ ID NO: 165.

The RIT-IgG2-ELLGG-H429F (a.k.a. RIT-IgG2-LLGG HF) heavy chain polypeptide comprised the CD20-specific heavy chain polypeptide of RIT-IgG2-ELLGG with a substitution of histidine with phenylalanine at position 429 in the CH3 region (H429F; Eu numbering). The amino acid sequence is shown in SEQ ID NO: 166 and is encoded by the codon-optimised DNA with the sequence shown as SEQ ID NO: 167.

### Expression of Antibodies by Expi293F Cells.

Expression of IgG antibodies was conducted using transient transfection of Expi293F cells (Thermo Fisher Scientific). Expi293F cells were cultured in Expi293 Expression Medium (Life Technologies) for both maintenance and protein production. Transfection of the cells was performed at RT using the Life Technologies Expifectamine Transfection Kit according to the manufacturer's instructions. Briefly, cells were transfected simultaneously with IgG heavy chain plasmid (15 μg) and light chain plasmid (15 μg) diluted in Opti-MEM (Gibco) and were then cultured at 37 °C for 16 to 18 hours. After this, 150 μL of the manufacturer's Enhancer 1 and 1.5 ml of Enhancer 2 were added and the cells cultured for a further five days at 34 °C Culture supernatants were then clarified by centrifugation at 3000×g for 40 minutes, and the supernatant filtered with a 0.2 μm high flow filter (Sartorius) prior to purification. The presence of expected antibody in the supernatant was confirmed by SDS-PAGE.

Protein A Affinity Purification of mAbs.

The mAbs were purified from the supernatant of the transfected Expi293F cells by protein A affinity chromatography. Briefly, Hi-trap™ Protein A high-performance columns (GE Healthcare Life Sciences) were washed and equilibrated in binding buffer (20 mM NaH₂PO₄ pH 7.0). Clarified cell culture supernatant was loaded, the columns washed with binding buffer to baseline OD280 nm, and bound antibody was eluted with 0.1M sodium citrate tribasic dihydrate (pH 3.5) and the eluate collected in 1 mL fractions. The fractions were neutralised immediately with 1M Tris-HCl pH 9.0 and fractions containing antibody were pooled.

Size Exclusion Chromatography (SEC) of mAbs.

Following protein A affinity purification, size exclusion chromatography (SEC) was used to further purify and characterise the antibodies. The protein A purified antibodies were concentrated (typically within a concentration range of 5-10 μg/mL) using a 30 kDa molecular weight cut-off centrifuge concentrator device (Merck-Millipore). Superose 6 Increase 10/300GL columns (GE Healthcare Life Sciences) were equilibrated in PBS pH 7.2, then the concentrated protein A affinity purified antibody was loaded and separated at a flow rate of 0.5 m/min in PBS. Fractions of 0.5 mL were collected and monomeric IgG peak fractions were pooled for subsequent use. Purified antibodies were confirmed by SDS-PAGE analysis.

### Evaluation of Antigen Binding by Monoclonal Antibodies.

Purified antibodies were tested for binding directly to target antigen (CD20) expressed on Ramos cells by flow cytometry as previously described in Trist et al., 2014 supra. Briefly, mAbs were titrated by serial 2-fold dilution, starting at a concentration of 2 μg/mL or 20 μg/mL in 25 μL of L15 buffer (Lebowitz-15 medium containing 0.5% BSA). Twenty-five microliters of cells at a concentration of 5×10⁶ cells/mL were then added to the titrated mAb and incubated for 30 minutes on ice. Cells were then washed twice in L15 buffer following centrifugation (200×g for 5 minutes at 4 °C), resuspended with 50 μL of FITC conjugated F(ab′)₂ fragments of goat anti-human IgG Fc, and incubated for 30 minutes on ice in the dark. Cells were next washed twice in cold L15 buffer, resuspended in 50 μL of 1% paraformaldehyde in PBS, and incubated for 10 minutes at RT in the dark. Cells were then washed once in L15 buffer and resuspended in 200 μL L15 buffer to be analyzed by flow cytometry using a BD FACSCanto™ II flow cytometer. Competitive binding assay to assess relative binding by monoclonal antibodies.

The binding of the human IgG1, IgG2, IgG3 and IgG4 subclasses to CD20 antigen was also compared using a competitive binding assay that detects the binding of mAb to a target antigen by competition with a biotinylated IgG1 antibody against the same target epitope. Briefly, mAbs were titrated by serial 2-fold dilution, starting at a concentration of 20 μg/mL, and were incubated with CD20-expressing Daudi cells on ice for 30 minutes in L15 buffer (Lebowitz-15 medium containing 0.5% BSA). Cells were centrifuged (200×g for 5 minutes at 4 °C) and unbound antibody was washed away in L15 buffer before 5 μg/mL of biotinylated RIT-IgG1-WT was added for a further 30 minutes on ice. Cells were centrifuged and washed twice in cold PBS to remove traces of biotin that may be present in the media, resuspended with 50 μL of streptavidin-FITC (BD Pharmingen) diluted in PBS, and incubated for 30 minutes on ice. Cells were then washed in cold PBS, resuspended in 50 μL of 1% paraformaldehyde in PBS, and incubated for 10 minutes at RT in the dark. Cells were then centrifuged and washed once in L15 buffer and resuspended in 200 μL L15 buffer to be analyzed by flow cytometry using a BD FACSCanto™ II flow cytometer.

Biotinylation of RIT-IgG1-WT (1-10 mg/mL, 1 mL) was performed by the addition of 27 μL of 10 mM EZ-Link™ Sulfo-NHS-LC-Biotin (Thermo Scientific; sulfosuccinimidyl-6-[biotin-amido]hexanoate) to 1 mL of mAb at a concentration of 1-10 mg/mL in PBS, followed by incubation at room temperature. Unbound biotinylation reagent was removed by dialysis against PBS pH 7.4 and antibody concentration was determined by an absorbance at 280 nm of 1.4 being equivalent to 1 mg/mL.

### Complement Dependent Lysis of Cells.

The ability of mAbs to induce target cell killing in the presence of serum was determined using a CDC assay. Cell viability was measured by flow cytometry using the Zombie Green Fixable Viability kit (BioLegend) following opsonisation of target Ramos cells with mAb. MAbs were titrated by serial 2-fold dilution, starting at a concentration of 2 μg/mL or 40 μg/mL in 25 μL of L15 buffer (Lebowitz-15 medium containing 0.5% BSA). Twenty-five microliters of Ramos cells at a concentration of 5×10⁶ cells/mL were then added to the titrated mAb and incubated for 30 minutes on ice. Cells were then centrifuged (200×g for 5 minutes at 4 °C) and washed twice in L15 buffer and resuspended in 50 μL of normal human serum (thawed and diluted 1:3 in L15 buffer immediately before use) and incubated for 30 minutes at 37 °C Following the incubation, the cells were centrifuged and washed twice in cold PBS then resuspended in 50 μL Zombie Green (prepared in DMSO according to the manufacturer's instructions; BioLegend) diluted 1:500 in PBS and incubated for a further 30 minutes on ice, protected from light. Cells were then centrifuged and washed once in PBS, resuspended in 50 μL of 1% paraformaldehyde in PBS, and incubated for 10 minutes at RT in the dark. Cells were then centrifuged and washed once in L15 buffer and resuspended in 200 μL L15 buffer to be analyzed by flow cytometry using a BD FACSCanto™ II flow cytometer.

### Results and Discussion

### The Human IgG2 Subclass Exhibits Reduced Binding to CD20 Antigen.

The purified anti-CD20 IgG1 and IgG2 mAbs were tested for binding to CD20 positive Daudi cells using a competition assay. Here, the binding of each mAb is determined for its ability to compete with an anti-CD20 mAb formatted with a wildtype H chain of human IgG1 (RIT-IgG1-WT) that has been labelled with biotin (RIT-IgG1-WT-biotin). Results indicated that RIT-IgG2-WT is unable to bind to target cells as efficiently as RIT-IgG1-WT since it was the weaker inhibitor of RIT-IgG1-WT-biotin (FIG. 62A). Despite their identical variable domains, the IgG2 subclass exhibited a substantial reduction in binding capacity compared to IgG1. Furthermore, this reduced ability to bind to target cells was found to be unique to the IgG2 subclass when compared to human IgG1, IgG3 and IgG4 (FIG. 62B). For example, whilst human IgG1, IgG3, and IgG4 subclasses exhibited equivalent binding profiles to target Daudi cells (IC50 values as follows: IgG1=34 nM, IgG3=32 nM, IgG4=33 nM), IgG2 displayed a markedly lower binding capacity (IC50=73 nM).

The binding of the RIT-IgG1 and RIT-IgG2 subclasses with or without the H429F mutation to target Ramos cells was determined directly via detection with a fluorescently labelled anti-Fc antibody (FIG. 63A). Both RIT-IgG1-WT and RIT-IgG1-H429F mAbs demonstrated equivalent binding, whilst the binding of the WT or H429F IgG2 (RIT-IgG2-WT and RIT-IgG2-H429F) was much lower than the IgG1 equivalents (FIG. 63A). Similarly, the IgG2 mutants comprising the lower hinge residues of IgG4 (RIT-IgG2-EFLGG and RIT-IgG2-EFLGG-H429F, a.k.a. RIT-IgG2-FLGG and RIT-IgG2-FLGG-H429F) or comprising the lower hinge residues of IgG1 (RIT-IgG2-ELLGG and RIT-IgG2-ELLGG-H429F, a.k.a. RIT-IgG2-LLGG and RIT-IgG2-LLGG-H429F) bound equivalently as RIT-IgG2-WT (FIG. 63B). Introduction of H429F did not negatively impact IgG1 or IgG2 binding to the CD20 antigen.

Evaluation of Target Cell Killing Potency by Anti-CD20 IgG2 mAbs with Lower Hinge and/or H429F Mutations.

The target cell killing potency mediated by the IgG1 and IgG2 subclasses formatted with or without the H429F mutation was first determined using CD20-expressing Ramos cells in the presence of normal human serum (FIG. 64). Cells were incubated in the presence of serial two-fold dilutions of mAb starting from 2 μg/mL, before normal human serum was added as a source of complement and the proportion of cells killed at each mAb concentration was determined by flow cytometry (FIG. 64A). Both RIT-IgG2-WT and RIT-IgG2-H429F failed to mediate detectable killing of Ramos cells above the background levels of lysis (6%) in the complement only control. In contrast, H429F substitution in the IgG1 subclass (RIT-IgG1-H429F) showed a marked improvement in mediating cell kill compared to the equivalent concentrations of the parental RIT-IgG1-WT mAb. Notably, as shown in FIG. 63A, both IgG1 mAbs demonstrated equivalent binding across the concentration range used in the CDC assay, indicating that the enhancement of killing potency by the H429F mutation is not attributable to differences in the binding of targets by the WT and H429F forms, but rather the presence of the H429F mutation. However, the less effective binding of the IgG2 subclass to efficiently bind to target Ramos cells, compared to IgG1, may contribute to the lack of difference between the killing potency of the WT and H429F forms for this subclass.

The effect of lower hinge modification in the IgG2 subclass on target cell killing potency was next examined (FIG. 64B). The concentration range examined in this assay commenced at 40 μg/mL of mAb treatment to ascertain whether any differences in cell killing could be detected at a higher mAb concentration. All IgG2 mAbs without the H429F substitution failed to mediate detectable killing of Ramos cells, regardless of the lower hinge residues that were present, however an increase in target cell killing was observed upon introduction of the H429F substitution into the heavy chain. This enhanced killing was more prominent for IgG2 mAbs comprising the sequence from the lower hinge of IgG4 (RIT-IgG2-EFLGG-H429F, a.k.a. Rit-IgG2-FLGG HF) or from IgG1 (RIT-IgG2-ELLGG-H429F, a.k.a. Rit-IgG2-LLGG HF). For example, at 40 μg/mL of mAb treatment, RIT-IgG2-H429F demonstrated a kill of 14%, whereas RIT-IgG2-EFLGG-H429F showed a kill of 44% and RIT-IgG2-ELLGG-H429F showed a kill of 39% (FIG. 64B). Furthermore, the EC50 of RIT-IgG2-EFLGG-H429F was 13.52 μg/mL, whilst the EC50 of RIT-IgG2-ELLGG-H429F was 34.21 μg/mL. This suggests that the IgG4 lower hinge residues are more permissive of inducing target cell killing, and subsequently have an advantage, in an IgG2 backbone when paired with the H429F substitution. Importantly, this marked enhancement of killing potency in IgG2 lower hinge mutants with H429F occurred despite the poor binding activity of these mAbs to target cells (FIG. 63B). Therefore, it is likely that improving the binding abilities of these mAbs will further enhance their target cell killing capabilities.

### Conclusion

The data in this example demonstrates that the human IgG2 subclass binds less efficiently to cell surface antigen, CD20, than other human IgG subclasses which thereby limits its ability to induce efficient lysis of target cells. Despite the poor binding activity of IgG2, an improvement in target cell killing was observed when the H429F substitution was incorporated into the heavy chain, particularly when the H429F substitution was incorporated with the substitution of the corresponding lower hinge sequence of IgG1 (RIT-IgG2-ELLGG-H429F) or more particularly with the corresponding lower hinge sequence of IgG4 (RIT-IgG2-EFLGG-H429F). If the ability of IgG2 to bind to cell surface antigen is increased, the incorporation of a permissive lower hinge sequence, such as EFLGG, may result in further enhancement of target cell killing potency.

### Example 19Activity Analysis of Anti-CD38 IgG2 mAbs with Upper Hinge and Lower Hinge Mutations

### Introduction

The most sequence diverse areas of immunoglobulins outside the variable domains are the hinge sequences as seen in the alignments of amino acid sequence of the constant regions and hinges of the Ig heavy chains, (FIG. 3). It should also be noted the CH2 domain sequence also incorporates sequences that are considered structurally as part of the hinge region of the IgG protein, and which is referred to in the field and herein as the lower hinge e.g. APELLGG of IgG1 and IgG3, APEFLGG of IgG4 and APPV-AG of IgG2. A more detailed alignment of the hinges of the human IgG subclasses is shown for clarity (Tables 7, 8 and 9) and show the hinges comprise an upper hinge, core hinge and lower hinge.

The human IgG2 heavy chain is unique as it is the only IgG subclass wherein the upper hinge is constrained as a result of inter- and intra-heavy chain disulphide bonds and the light chain can attach in one of three positions—cysteine 131 in the CH1 domain, or cysteine 219 and cysteine 220 in the upper hinge (Table 7, FIG. 65).

Thus, in addition to lacking key residues in the lower hinge that are associated with C1q and FcγR interactions, the hinge of IgG2 is more constrained than other IgG subclasses due to the presence of two more cysteine residues (cysteine 219 and cysteine 220) that enable the formation of disulfide bonds between the heavy chains, including cysteine 131 in the CH1 domain, or between the light chain and heavy chain (Dillon et al., J Biol Chem 283(23):16206-16215, 2008). The differential bonding of these cysteine residues produces three distinct isoforms of IgG2 (IgG2-A, -B, and -A/B), each producing different conformations of the Fab arms relative to the hinge with varying degrees of rigidity (Dillon et al., 2008 supra). This has important implications for target binding and function, with the least constrained configuration, IgG2-A, reported to demonstrate a higher target affinity for surface antigens (Dillon et al., 2008 supra). Thus, whilst the absence of key residues in the lower hinge of IgG2 may impair its ability to bind to effector molecules such as C1q, the presence of additional disulfide bonds in the upper hinge may also reduce the flexibility of the Fab arms and function to impede antigen binding or effector molecule engagement.

In this example, a series of mutations were introduced into the upper and lower hinge of IgG2. To investigate whether the IgG2 subclass shows reduced binding to targets other than CD20, the mAbs in this example are directed against CD38 using the variable domain of isatuximab. Firstly, several cysteine-to-serine mutations in the IgG2 upper hinge were introduced to investigate whether the disruption of disulfide bonds to provide only a single light chain binding site lessens the constraints imposed by heavy chain disulphide bonding and improves the antigen binding capabilities of IgG2, as represented in FIG. 65. Secondly, residues in the IgG2 lower hinge (PV-AG) were substituted with the residues (EFLGG) from the lower hinge of IgG4 to investigate the effect on complement activation and lysis of target cells, also as represented in FIG. 65.

### Methods and Materials

### Antibodies and Antibody Constructs.

All anti-CD38 mAbs used in this example carried the variable light chain (VL) and VH domains of isatuximab.

The light chain polypeptide comprising the CD38-specific VL domain of the isatuximab fused to human kappa constant domain comprised the amino acid sequence shown as SEQ ID NO: 44, that is encoded by the codon-optimised DNA with the sequence shown as SEQ ID NO: 45.

The heavy chains of the mAbs were formatted as human IgG1 or human IgG2 subclass H chains. These chains were either unmodified (WT) heavy chains or mutated heavy chains that contained one or more amino acid sequence changes. The heavy chain sequences of the mAbs used in this example are described below and listed with their sequence IDs in Table 10 below.

TABLE 10
Anti-CD38 mAbs generated carrying various mutations within the IgG2
heavy chain of Isatuximab

| | | | Region/Amino Acid position(s) | | | | | | | |
|---|---|---|---|---|---|---|---|---|---|---|
| mAb HC | protein | DNA | CH1/ | UH/ | UH/ | LH/ | CH2/ | CH2/ | CH2/ | CH3/ |
| Name | Seq ID | Seq ID | 131 | 219 | 220 | 233-237 | 268 | 274 | 327 | 429 |
| Isa-IgG1- | 42 | 43 | S | S | C | ELLGG | H | K | A | H |
| WT | | | | | | | | | | |
| Isa-IgG2- | 184 | 185 | C | C | C | PV-AG | H | Q | A | H |
| WT | | | | | | | | | | |
| Isa-IgG2- | 186 | 187 | S | C | S | EFLGG | H | Q | A | H |
| M1 | | | | | | | | | | |
| Isa-IgG2- | 188 | 189 | S | S | C | EFLGG | H | Q | A | H |
| M2 | | | | | | | | | | |
| Isa-IgG2- | 190 | 191 | S | S* | C* | EFLGG | H | Q | A | H |
| M3* | | | | | | | | | | |
| Isa-IgG2- | 192 | 193 | S | S | C | PV-AG | H | Q | A | H |
| C131S- | | | | | | | | | | |
| C219S | | | | | | | | | | |
| Isa-IgG2- | 194 | 195 | C | C | C | EFLGG | H | Q | A | H |
| EFLGG | | | | | | | | | | |

HC = heavy chain, CH1 = constant domain 1 of the HC, UH = upper hinge, LH = lower hinge (N-terminus of the CH2 domain).
All amino acid positions are numbered according to the Eu numbering system that aligns heavy chain residues to that of a wildtype human IgG1 wildtype sequence, as shown in FIG. 3 and FIG. 4 (Table 7 and Table 9).
*NOTE that Isa-IgG2-M3 contains a substitution of the entire upper hinge sequence of human IgG2 (ERKCCVE) with the corresponding upper hinge sequence from human IgG1 (EPKSCDKTHT; residues 216-225 in the Eu numbering system, Table 7).

The isatuximab-IgG1-WT mAb (Isa-IgG1-WT a.k.a. Isa-WT or isatuximab heavy chain) used in the example comprised a CD38 specific heavy chain polypeptide comprising, in the order of the N-terminus to the C-terminus, the CD38-specific VH domain of isatuximab fused to the CH1-hinge-CH2-CH3 domains of human IgG1 (SEQ ID NO: 42), and is encoded by the codon-optimised DNA with the sequence shown as SEQ ID NO: 43).

The isatuximab-IgG2-WT mAb (Isa-IgG2-WT) used in the example comprised a CD38 specific heavy chain polypeptide comprising, in the order of the N-terminus to the C-terminus, the CD38-specific VH domain of isatuximab fused to the CH1-hinge-CH2-CH3 domains of human IgG2 (SEQ ID NO: 184), and is encoded by the codon-optimised DNA with the sequence shown as SEQ ID NO: 185).

The isatuximab-IgG2-M1 mAb (Isa-IgG2-M1) used in the example comprised the CD38 specific heavy chain polypeptide of Isa-IgG2-WT with the following mutations: the cysteine at position 131 in CH1 was substituted with serine (C131S; Eu numbering), the cysteine at position 220 in the upper hinge was substituted with serine (C220S; Table 7, Eu numbering) and the residues at position 233-237 of IgG2 (PV-AG) in the lower hinge were substituted with the amino acids 233-237 (EFLGG) from the lower hinge of the human IgG4 subclass (Table 9). The amino acid sequence is shown in SEQ ID NO: 186 and is encoded by the codon-optimised DNA with the sequence shown as SEQ ID NO: 187.

The isatuximab-IgG2-M2 mAb (Isa-IgG2-M2) used in the example comprised the CD38 specific heavy chain polypeptide of Isa-IgG2-WT with the following mutations: the cysteine at position 131 in CH1 was substituted with serine (C131S; Eu numbering), the cysteine at position 219 in the upper hinge was substituted with serine (C219S; Table 7, Eu numbering) and the residues at position 233-237 of IgG2 (PV-AG) in the lower hinge were substituted with the amino acids 233-237 (EFLGG) from the lower hinge of the human IgG4 subclass (Table 9). The amino acid sequence is shown in SEQ ID NO: 188 and is encoded by the codon-optimised DNA with the sequence shown as SEQ ID NO: 189.

The isatuximab-IgG2-M3 mAb (Isa-IgG2-M3) used in the example comprised the CD38 specific heavy chain polypeptide of Isa-IgG2-WT with the following mutations: the residues 216-225 (ERKCCVE; Eu numbering) in the IgG2 upper hinge were substituted with residues 216-225 of the IgG1 subclass (EPKSCDKTHT; Table 7, Eu numbering), and additionally the residues 233-237 in the lower IgG2 hinge (PV-AG) were substituted with residues 233-237 (EFLGG) from the IgG4 subclass (Table 9). The amino acid sequence is shown in SEQ ID NO: 190 and is encoded by the codon-optimised DNA with the sequence shown as SEQ ID NO: 191.

The isatuximab-IgG2-C131S-C219S mAb (Isa-IgG2-C131S-C219S) used in the example comprised the CD38 specific heavy chain polypeptide of Isa-IgG2-WT with following mutation: the cysteine at position 219 in the upper hinge was substituted with serine (C219S; Table 7, Eu numbering). The amino acid sequence is shown in SEQ ID NO: 192 and is encoded by the codon-optimised DNA with the sequence shown as SEQ ID NO: 193.

The isatuximab-IgG2-EFLGG mAb (Isa-IgG2-EFLGG) used in the example comprised the CD38 specific heavy chain polypeptide of Isa-IgG2-WT with the following mutation: the residues at position 233-237 of IgG2 (PV-AG) in the lower hinge were substituted with the amino acids (EFLGG) from the lower hinge of the human IgG4 subclass (Table 7). The amino acid sequence is shown in SEQ ID NO: 194 and is encoded by the codon-optimised DNA with the sequence shown as SEQ ID NO: 195.

The anti-CD20 rituximab-IgG1-WT mAb (RIT-IgG1-WT) used in this example carried the V_{L} and V_{H} domain of rituximab.

The RIT-IgG1-WT light chain polypeptide comprised of the amino acid sequence as shown in SEQ ID NO: 30 (comprising the CD20-specific VL domain of the rituximab mAb fused to human kappa constant domain) and is encoded by the codon-optimised DNA with the sequence shown as SEQ ID NO: 31.

The RIT-IgG1-WT heavy chain polypeptide comprised, in the order of the N-terminus to the C-terminus, the CD20-specific VH domain of rituximab fused to the CH1-hinge-CH2-CH3 domains of human IgG1, as shown in SEQ ID NO: 28, and is encoded by the codon-optimised DNA with the sequence shown as SEQ ID NO: 29.

### Antibody Expression Vectors

The CD38 mAbs were produced using the antibody expression vector pcDNA3.4, as is known in the art. Briefly, antibody expression vectors consisted of a synthetic polynucleotide sequence, encoding the antibody heavy chain or light chain, appropriately placed within pcDNA3.4 (Thermo Fisher Scientific).

### Expression and Purification of Antibodies by Expi293F Cells.

The mAbs were purified from supernatant of the transfected Expi293F cells by Protein A affinity chromatography as described in Example 3. Following Protein A affinity purification, size exclusion chromatography (SEC) was used to further purify and characterise the antibodies as described in Example 3.

Purified antibodies were confirmed by SDS-PAGE analysis.

### Evaluation of Antigen Binding by Monoclonal Antibodies.

The anti-CD38 mAbs were tested for direct binding on Raji lymphoma cells expressing CD38. Briefly, mAbs were titrated by serial 3-fold dilution, starting at a concentration of 5 μg/mL in 25 μL of FACS buffer (PBS with 0.5% (w/v) BSA). Twenty-five microliters of cells at a concentration of 5×10⁶ cells/mL were then added to the titrated mAb and incubated for 30 minutes on ice. Cells were then washed twice in FACS buffer following centrifugation (200×g for 5 minutes at 4 °C), resuspended in 50 μL of FITC conjugated F(ab′)₂ fragments of goat anti-human IgG Fc, and incubated for 30 minutes on ice in the dark. Cells were next washed twice in cold FACS buffer, resuspended in 50 μL of 1% paraformaldehyde in PBS, and incubated for 10 minutes at RT in the dark. Cells were then washed once in FACS buffer and resuspended in 200 μL FACS buffer to be analyzed by flow cytometry using a BD FACSCanto™ II flow cytometer.

### Competitive Binding Assay to Assess Relative Binding by Monoclonal Antibodies.

The binding of purified mAbs to CD38 antigen was also determined using a competitive binding assay that detects the binding of mAb to a target antigen by competition with a biotinylated IgG1 antibody against the same target epitope. Briefly, mAbs were titrated by serial 3-fold dilution, starting at a concentration of 10 μg/mL, and were incubated with Raji cells on ice for 30 minutes in L15 buffer (Lebowitz-15 medium containing 0.5% BSA). Cells were centrifuged (200×g for 5 minutes at 4 °C) and unbound antibody was washed away in L15 buffer before 5 μg/mL of biotinylated Isa-IgG1-WT was added for a further 30 minutes on ice. Cells were centrifuged and washed twice in cold PBS to remove traces of biotin that may be present in the cell culture media or L15-buffer, resuspended with 50 μL of FITC-conjugated streptavidin (BD Pharmingen) diluted in PBS, and incubated for 30 minutes on ice. Cells were then washed in cold PBS resuspended in 50 μL of 1% paraformaldehyde in PBS and incubated for 10 minutes at RT in the dark. Cells were then centrifuged and washed once in L15 buffer and resuspended in 200 μL L15 buffer to be analysed by flow cytometry using a BD FACSCanto™ II flow cytometer.

Biotinylation of Isa-IgG1-WT (1-10 mg/mL, 1 mL) was performed by the addition of 27 μL of 10 mM EZ-Link™ Sulfo-NHS-LC-Biotin (Thermo Scientific; sulfosuccinimidyl-6-[biotin-amido]hexanoate) to 1 mL of mAb at a concentration of 1-10 mg/mL in PBS, followed by incubation at room temperature. Unbound biotinylation reagent was removed by dialysis against PBS pH 7.4 and antibody concentration was determined by an absorbance at 280 nm of 1.4 being equivalent to 1 mg/mL.

### Complement Dependent Cytotoxicity of Cells

Cell killing (CDC) in the presence of normal human serum as a source of complement was measured by flow cytometry using the Zombie Green Fixable Viability kit (BioLegend) that stains the primary amines of intracellular proteins in non-viable cells with compromised membranes. The mAbs were titrated by serial 3-fold dilution, starting at a concentration of 5 μg/mL in 25 μL of L15 buffer (Lebowitz-15 medium containing 0.5% BSA). Twenty-five microliters of target cells at a concentration of 5×10⁶ cells/mL were then added to the titrated mAb and incubated for 30 minutes on ice. Cells were then centrifuged (200×g for 5 minutes at 4 °C) and washed twice in L15 buffer and resuspended in 50 μL of normal human serum (thawed and diluted 1:3 in L15 buffer immediately before use) and incubated for 30 minutes at 37 °C Following the incubation, the cells were centrifuged and washed twice in cold PBS then resuspended in 50 μL Zombie Green (prepared in DMSO according to the manufacturer's instructions) diluted 1:500 in PBS and incubated for a further 30 mins on ice, protected from light. Cells were then centrifuged and washed once in PBS, resuspended in 50 μL of 1% paraformaldehyde in PBS, and incubated for 10 minutes at RT in the dark, washed once in L15 buffer and resuspended in 200 μL L15 buffer. Cells were then centrifuged and washed and then resuspended in 200 T L15 buffer to be analyzed by flow cytometry using a BD FACSCanto™ TT flow cytometer. Non-viable cells were identified by positive staining with Zombie Green Fixable Viability dye.

Detection of Bound C1 to mAb Opsonized Target Cells with an Anti-C1_{q} Detection Antibody.

C1 binding to antibody-opsonised cells was evaluated by flow cytometry. As Raji cells express surface receptors for C1q, Ramos lymphoma cells were used for this assay (5×10⁶ cells/mL in Dulbecco's phosphate-buffered saline (in Dulbecco's phosphate-buffered saline (DPBS)+0.5% BSA) and were incubated with 5 μg/mL of mAb on ice for 30 minutes. Cells were centrifuged (200×g for 5 minutes at 4 °C) and washed twice in DPBS+0.5% BSA before they were resuspended in normal human serum (NHS) diluted 1 in 3 in DPBS+0.5% BSA and incubated on ice for a further 30 minutes. The cells were then washed once in cold DPBS+0.5% BSA, resuspended in polyclonal-rabbit anti-C1q antiserum (Burnet Institute) diluted 1:100 in DPBS+0.5% BSA, and incubated for a further 30 minutes on ice. Cells were then centrifuged and washed once in cold DPBS+0.5% BSA, resuspended in 50 μL of APC-conjugated donkey antiserum detecting rabbit IgG (H+L) (Jackson ImmunoResearch), and incubated for a further 30 minutes on ice, protected from light. Cells were then centrifuged and washed once in DPBS, resuspended in 50 μL of 1% paraformaldehyde in PBS, and incubated for 10 minutes at RT in the dark. Cells were then washed once in DPBS and resuspended in 200 μL DPBS+0.5% BSA+1 mM EDTA to be analyzed by flow cytometry using a BD FACSCanto™ II flow cytometer. The amount of C1 bound to the mAb opsonised target cells was determined by degree of anti-C1q detected with the fluorescent labelled secondary antibody, and is represented as the median fluorescence intensity (MFI)

### Results and Discussion

### Isatuximab IgG2 Upper and Lower Hinge Mutants Display Enhanced Binding to Target Cells.

Anti-CD38 IgG1 and IgG2 mAbs were produced as described above and purified separately by protein A affinity chromatography followed by size exclusion chromatography (SEC). SEC elution profiles demonstrated that all mAbs contained a single monomeric IgG (H₂L₂) species with minimal oligomeric species (FIG. 66). The fractions containing monomeric IgG were pooled and the purified mAbs were examined by SDS-PAGE analysis (FIG. 67). This revealed the expected ~150 kDa H₂L₂ as the predominate species under non-reducing conditions. Thus, all the molecules formed disulfides between, heavy chain and heavy chain (H-H), and between heavy chain and light chain (H-L). The absence of free light chain confirms the disulfide bonding patterns for the M1, M2 and M3 mutants indicated in FIG. 65. All the antibodies, upon disulfide bond reduction in dithiothreitol, resolved to the expected ~50 kDa heavy chain and ~25 kDa light chain species.

The purified anti-CD38 mAbs were tested by flow cytometry for binding to CD38-expressing Raji cells. Binding was first directly determined via detection of bound mAb with a fluorescently labelled anti-Fc antibody. Comparison of the IgG1-WT and IgG2-WT mAbs revealed significantly reduced binding to target Raji cells by the IgG2 subclass compared to the IgG1 subclass (FIG. 68A).

The hinge region modified IgG2 mAbs (Isa-IgG2-M1, Isa-IgG2-M2, and Isa-IgG2-M3) displayed a significant improvement in binding compared to unmodified Isa-IgG1-WT mAb (FIG. 68B). The greatest improvement in binding was observed for Isa-IgG2-M2 (p<0.0001) and Isa-IgG2-M3 (p<0.0001), and Isa-IgG2-M1 displayed an improvement in binding compared to Isa-IgG2-WT, although less significant than the other two mutants (p=0.0327). This suggests the mutations introduced into the upper and lower hinge of these IgG2 mutants improves the target binding capabilities of IgG2.

Lastly, no significant improvements in binding were found for Isa-IgG2-C131S-C219S, in which the upper hinge is less constrained through the disruption of disulphide bonding but the IgG2 lower hinge is retained, or Isa-IgG2-EFLGG that comprises residues from the IgG4 lower hinge but retains the constrained upper hinge of IgG2 (FIG. 68C).

Importantly, the presence of structural isoforms in human IgG2 can mask the binding sites for anti-Fc antibodies, as previously reported in a study by Lightle et al., Protein Sci 19(4):753-762, 2010. Therefore, to more directly address the differences in binding, a competition assay was used whereby the binding of each mAb is determined for its ability to compete with anti-CD38 mAb formatted with a wildtype H chain of human IgG1 (Isa-IgG1-WT) that has been labelled with biotin (Isa-IgG1-WT-biotin). Results confirmed that Isa-IgG2-WT does not compete as well as Isa-IgG1-WT despite their identical variable domains (FIG. 69A). All hinge region modified IgG2 mAbs (Isa-IgG2-M1, Isa-IgG2-M2, and Isa-IgG2-M3) competed better than unmodified Isa-IgG2-WT, indicating a significant improvement in binding capability (p<0.0001) (FIG. 69B). Isa-IgG2-C131S-C219S and Isa-IgG2-EFLGG did not show significant improvement in their ability to compete with the biotinylated Isa-IgG1-WT compared to Isa-IgG2-WT in this assay (FIG. 69C). In all experiments, pre-incubation with a non-competing antibody (RIT-IgG1-WT) that binds to CD20 had no effect on the binding of Isa-IgG1-WT-biotin, highlighting the specificity of the assay.

The results from both direct binding and competition assays show that, whilst the IgG2 subclass is less efficient in its ability to bind surface antigen, the surprising combination of lower and upper hinge mutations (in mutants M1, M2, and M3 (as depicted if FIG. 65) increases the binding capacity of IgG2.

### Isatuximab IgG2 Upper and Lower Hinge Mutants Show Enhanced CDC of Target Cells.

The effect on the hinge mutations on the ability of IgG2 to induce lysis of target cells was evaluated using a complement dependent cytotoxicity (CDC) assay. CD38-expressing Raji cells were incubated in the presence of serial three-fold dilutions of mAb before normal human serum was added as a source of complement and the proportion of cells killed at each mAb concentration was determined by flow cytometry. Comparison of the Isa-IgG1-WT and Isa-IgG2-WT revealed that the IgG2 subclass elicits a weaker cytotoxic response compared to IgG1 (FIG. 70A). All hinge region modified IgG2 mAbs (Isa-IgG2-M1, Isa-IgG2-M2, and Isa-IgG2-M3) exhibited enhanced target cell killing compared to that by unmodified Isa-IgG2-WT for which the target cell killing at 5 μg/mL was only 10% kill (FIG. 70B). The Isa-IgG2-M1 and Isa-IgG2-M2 were equipotent and showed the largest increase in potency, Isa-IgG2-M1 (34% kill at 5 μg/mL and EC50=1.09 nM) and Isa-IgG2-M2 (33% kill at 5 μg/mL and EC50=1.71 nM) and Isa-IgG2-M3 which contains the entire upper hinge of IgG1 showed the smallest increase 19% kill at 5 μg/mL and EC50=1.52 nM. All three mAbs contain lower hinge sequences from IgG4 and the C131S mutation in the CH1 domain. The M1 and M2 mAbs also contain specific point mutations, C220S or C219S, in the IgG2 upper hinge that provides only a single light chain binding site and removes constraints imposed by heavy chain disulphide bonding. Interestingly M3 showed the smallest increase in CDC yet the only difference between M3 and M1 or M3 and M2 is that M3 contains the entire upper hinge of IgG1 which also has a single light chain disulphide bond, due to the presence of C220, but is otherwise distinct in amino acid composition from the IgG2 upper hinge. The upper hinge of IgG1 is 3 amino acids longer than that of IgG2 (Table 7). This suggests that the unconstrained IgG2 upper hinge sequence may, in the context of IgG2 backbones, confer some advantage over the IgG1 upper hinge in killing assays.

In further analysis the contribution of the upper and lower hinge modifications to the enhanced CDC were investigated in separate mutant IgG mAbs. In Isa-IgG2-C131S-C219S the site of H-L disulfide bonding on the H-chain is confined to C220 and its formation is confirmed by SDS-PAGE. In this context of L-chain disulfide attachment to the H-chain these mutations eliminate any intra-H chain disulfides, either between C131 and C219, or C131 and C220, which occur naturally in some forms of IgG2 (Dillon et al., 2008 supra). Thus, the upper hinge of Isa-IgG2-C131S-C219S is less constrained compared to IgG2-WT. Since the upper hinge affects segmental flexibility of IgG antibodies, the Isa-IgG2-C131S-C219S mutant form of IgG2 is, overall, more flexible that IgG2-WT. This C131S-C219S dual mutation will likewise confer greater flexibility to the M1 mutant mAb (FIG. 65). Notably Isa-IgG2-C131S-C219S showed no improvement in CDC compared to unmodified Isa-IgG2-WT (FIG. 70C). Thus, the C131S-C219S mutation, is unlikely to confer the increased CDC activity of the Isa-IgG2-M1 mutant. Similarly, the C131S-C220S mutations, are unlikely to confer the increased CDC activity of the Isa-IgG2-M2 mutant. By contrast the mAb Isa-IgG2-EFLGG, wherein only the lower hinge is modified by replacement with sequence from the IgG4 lower hinge and the native constrained upper hinge of IgG2 is retained, shows considerable enhancement of CDC compared to Isa-IgG2-WT (FIG. 70C). Thus, the substitution of the IgG2 lower hinge sequences with permissive lower hinge sequences such as IgG4 EFLGG potentiates the cytotoxic effect of this mutant Isa-IgG2, and similarly replacement with IgG1 ELLGG lower hinge sequence would also potentiate cytotoxic potency.

Additionally, the improvement in potency as seen for M1 and M2 mAbs that also comprise a modified IgG2 upper hinge is associated with improved target binding (FIG. 68B and FIG. 69B) suggesting that the modified IgG2 upper hinge in combination with a permissive lower hinge such as that from IgG4 or IgG1, yields potency greater than either alone. This is demonstrated at concentrations less than 1 μg/mL where M1 showed greater cytotoxicity than either the Isa-IgG2-EFLGG or Isa-IgG2-C131S-C219S (FIGS. 70B and 70C).

Lastly, the observed enhancement in target cell killing activity by the mAbs (+C′, FIG. 70D) was dependent on the presence of human serum containing complement proteins as no killing above background (no mAb) was observed when cells were treated with antibody in the absence of complement (−C′, FIG. 70D).

### Isatuximab IgG2 Upper and Lower Hinge Mutants Show Enhanced Deposition of C1 on Opsonized Target Cells.

CDC mediated by the classical pathway is initiated by the binding of the hexameric complement protein C1q, which is part of the C1 complex. The C1 complex is stabilised by the C1r and C1s proteases which aid C1q binding to IgG opsonised surfaces (Zwarthoff et al., 2021 supra). The binding of the C1 complex by antigen-bound mAb leads to the formation of the membrane attack complex (MAC) that induces target cell lysis, as measured by CDC assays. The ability of anti-CD38 mAbs to bind to C1 on the surface of CD38-expressing target Ramos cells was evaluated by flow cytometry (FIG. 71). Ramos cells were used in this assay as the Raji cell line expresses cell surface receptors for several complement components, including C1q (Ghebrehiwet et al., J Exp Med 160:1375-1389, 1984), that interfere with the C1 binding assay, where C1 binding is dependent on the C1q subcomponent and is measured using an anti-C1q antibody. C1 binding was considerably improved for all mutant mAbs comprising IgG4 lower hinge residues (Isa-IgG2-M1, Isa-IgG2-M2, Isa-IgG2-M3, and Isa-IgG2-EFLGG) compared to the unmodified Isa-IgG2-WT mAb. Additionally, the binding of C1q by these mutants was at least equivalent, if not greater, than Isa-IgG1-WT, reflective of the potency of each mAb as measured by CDC assay in (FIG. 71).

### Conclusion

The data obtained in this example demonstrates that the IgG2 subclass shows reduced binding to surface antigen CD38 when compared to the IgG1 subclass. The reduced binding capacity of IgG2 mAbs is not limited to CD38 as an antigen, as an impairment in CD20 binding by IgG2 was also shown previously in FIG. 62. Therefore, there is an inherent difference in the IgG2 subclass that impedes upon the ability of mAb to bind surface antigen that is not strictly dependent of the variable domain.

The reduced target binding of the IgG2 subclass is proposed to be a consequence of the differential disulfide bonding that occurs between the light and heavy chains, or within the heavy chains, in the upper hinge of IgG2 that constrains the Fab arms in more rigid conformations. The data in this example explores the use of mutations to disrupt the disulfide bonding pattern in the IgG2 upper hinge (via the following mutations: C131S/C220S or C131S/C219S). In the context of L-chain disulfide attachment to the H-chain these mutations eliminate any intra-H chain disulfide, either between C131 and C219, or C131 and C220, which occur naturally in some forms of IgG2. These disulfide mutants when paired with the permissive lower hinge residues from IgG4 (‘EFLGG’; residues 233-237 Eu numbering) improve the capability of IgG2 to bind to cell surface antigen and induce target cell killing. Therefore, these combined mutations in the IgG2 upper and lower hinge regions may be utilized in the development of therapeutic mAbs with enhanced effector functions.

### Example 20Activity Analysis of Anti-CD38 IgG2 mAbs with Lower Hinge and Upper Hinge Mutations in Addition to the H429F Mutation

In this example, the effect of the H429F mutation on the cell-killing potency of the hinge-region modified IgG2 anti-CD38 mAbs with the isatuximab variable domain (Isa-IgG2-M1, Isa-IgG2-M2, and Isa-IgG2-M3) was investigated. The construction of the mAbs and their sequence IDs are described below, and Table 11 below also describes characteristics of these antibodies.

TABLE 11
Anti-CD38 mAbs generated using the IgG1 or IgG2 heavy chains and
mutant heavy chains

| | | | Region/Amino Acid position(s) | | | | | | | |
|---|---|---|---|---|---|---|---|---|---|---|
| mAb HC | Protein | DNA | CH1/ | UH/ | UH/ | LH/ | CH2/ | CH2/ | CH2/ | CH3/ |
| Name | Seq ID | Seq ID | 131 | 219 | 220 | 233-237 | 268 | 274 | 327 | 429 |
| Isa-IgG1- | 42 | 43 | S | S | C | ELLGG | H | K | A | H |
| WT | | | | | | | | | | |
| Isa-IgG2- | 184 | 185 | C | C | C | PV-AG | H | Q | A | H |
| WT | | | | | | | | | | |
| Isa-IgG2- | 186 | 187 | S | C | S | EFLGG | H | Q | A | H |
| M1 | | | | | | | | | | |
| Isa-IgG2- | 188 | 189 | S | S | C | EFLGG | H | Q | A | H |
| M2 | | | | | | | | | | |
| Isa-IgG2- | 190 | 191 | S | S | C | EFLGG | H | Q | A | H |
| M3* | | | | | | | | | | |
| Isa-IgG2- | 194 | 195 | C | C | C | EFLGG | H | Q | A | H |
| EFLGG | | | | | | | | | | |
| Isa-IgG1- | 196 | 197 | S | S | C | ELLGG | H | K | A | F |
| H429F | | | | | | | | | | |
| Isa-IgG2- | 198 | 199 | C | C | C | PV-AG | H | Q | A | F |
| H429F | | | | | | | | | | |
| Isa-IgG2- | 200 | 201 | S | C | S | EFLGG | H | Q | A | F |
| M1-H429F | | | | | | | | | | |
| Isa-IgG2- | 202 | 203 | S | S | C | EFLGG | H | Q | A | F |
| M2-H429F | | | | | | | | | | |
| Isa-IgG2- | 204 | 205 | S | S | C | EFLGG | H | Q | A | F |
| M3- | | | | | | | | | | |
| H429F* | | | | | | | | | | |
| Isa-IgG2- | 206 | 207 | C | C | C | EFLGG | H | Q | A | F |
| EFLGG- | | | | | | | | | | |
| H429F | | | | | | | | | | |

HC = heavy chain, CH1 = constant domain 1 of the HC, UH = upper hinge, LH = lower hinge (N-terminus of the CH2 domain).
All amino acid positions are numbered according to the Eu numbering system that aligns heavy chain residues to that of a wildtype human IgGI wildtype sequence, as shown in FIG. 3 and FIG. 4 (Table 7 and Table 9).
*NOTE that Isa-IgG2-M3 and Isa-IgG2-M3-H429F contain a substitution of the entire upper hinge sequence of human IgG2 (ERKCCVE) with the corresponding upper hinge sequence from human IgG1 (EPKSCDKTHT; residues 216-225 in the Eu numbering system, Table 7).

### Methods and Materials

### Antibodies and Antibody Constructs.

All anti-CD38 mAbs used in this example carried the variable light chain (VL) and VH domains of isatuximab.

The light chain polypeptide comprising the CD38-specific VL domain of the isatuximab fused to human kappa constant domain comprised the amino acid sequence shown as SEQ ID NO: 44, that is encoded by the codon-optimised DNA with the sequence shown as SEQ ID NO: 45.

The heavy chains of the mAbs were formatted as human IgG1 or human IgG2 subclass H chains. These chains were either unmodified (WT) heavy chains or mutated heavy chains that contained one or more amino acid sequence changes. The heavy chain sequences of the mAbs used in this example are described below and listed with their sequence IDs in Table 11.

The isatuximab-IgG1-WT mAb (Isa-IgG1-WT) used in the example comprised a CD38 specific heavy chain polypeptide comprising, in the order of the N-terminus to the C-terminus, the CD38-specific VH domain of isatuximab fused to the CH1-hinge-CH2-CH3 domains of human IgG1 (SEQ ID NO: 42), and is encoded by the codon-optimised DNA with the sequence shown as SEQ ID NO: 43).

The isatuximab-IgG1-H429F mAb (Isa-IgG1-H429F) used in the example comprised a CD38 specific heavy chain polypeptide comprising, in the order of the N-terminus to the C-terminus, the CD38-specific VH domain of isatuximab fused to the CH1-hinge-CH2-CH3 domains of human IgG1 with a substitution of histidine with phenylalanine at position 429 (H429F; Eu numbering) in the CH3 region (SEQ ID NO: 196), and is encoded by the codon-optimised DNA with the sequence shown as SEQ ID NO: 197.

The isatuximab-IgG2-WT mAb (Isa-IgG2-WT) used in the example comprised a CD38 specific heavy chain polypeptide comprising, in the order of the N-terminus to the C-terminus, the CD38-specific VH domain of isatuximab fused to the CH1-hinge-CH2-CH3 domains of human IgG2 (SEQ ID NO: 184), and is encoded by the codon-optimised DNA with the sequence shown as SEQ ID NO: 185.

The isatuximab-IgG2-H429F mAb (Isa-IgG2-H429F) used in the example comprised a CD38 specific heavy chain polypeptide comprising, in the order of the N-terminus to the C-terminus, the CD38-specific VH domain of isatuximab fused to the CH1-hinge-CH2-CH3 domains of human IgG2 with a substitution of histidine with phenylalanine at position 429 (H429F; Eu numbering) in the CH3 region (SEQ ID NO: 198), and is encoded by the codon-optimised DNA with the sequence shown as SEQ ID NO: 199.

The isatuximab-IgG2-M1 mAb (Isa-IgG2-M1) used in the example comprised the CD38 specific heavy chain polypeptide of Isa-IgG2-WT with the following mutations: the cysteine at position 131 in CH1 was substituted with serine (C131S; Eu numbering), the cysteine at position 220 in the upper hinge was substituted with serine (C220S; Eu numbering) and the residues at position 233-237 of IgG2 (PV-AG) in the lower hinge were substituted with the amino acids (EFLGG) from the lower hinge of the human IgG4 subclass. The amino acid sequence is shown in SEQ ID NO: 186 and is encoded by the codon-optimised DNA with the sequence shown as SEQ ID NO: 187.

The isatuximab-IgG2-M1-H429F mAb (Isa-IgG2-M1-H429F) comprised the CD38 specific heavy chain polypeptide of isatuximab-IgG2-M1 mAb (Isa-IgG2-M1) with a substitution of histidine with phenylalanine at position 429 in the CH3 region (H429F; Eu numbering). The amino acid sequence is shown in SEQ ID NO: 200 and is encoded by the codon-optimised DNA with the sequence shown as SEQ ID NO: 201.

The isatuximab-IgG2-M2 mAb (Isa-IgG2-M2) used in the example comprised the CD38 specific heavy chain polypeptide of Isa-IgG2-WT with the following mutations: the cysteine at position 131 in CH1 was substituted with serine (C131S; Eu numbering), the cysteine at position 219 in the upper hinge was substituted with serine (C219S; Eu numbering) and the residues at position 233-237 of IgG2 (PV-AG) in the lower hinge were substituted with the amino acids (EFLGG) from the lower hinge of the human IgG4 subclass. The amino acid sequence is shown in SEQ ID NO: 188, and is encoded by the codon-optimised DNA with the sequence shown as SEQ ID NO: 189.

The isatuximab-IgG2-M2-H429F mAb (Isa-IgG2-M2-H429F) used in the example comprised the CD38 specific heavy chain polypeptide of Isa-IgG2-M2 mAb (Isa-IgG2-M2) with a substitution of histidine with phenylalanine at position 429 in the CH3 region (H429F; Eu numbering).

The amino acid sequence is shown in SEQ ID NO: 202, and is encoded by the codon-optimised DNA with the sequence shown as SEQ ID NO: 203.

The isatuximab-IgG2-M3 mAb (Isa-IgG2-M3) used in the example comprised the CD38 specific heavy chain polypeptide of Isa-IgG2-WT with the following mutations: the residues 216-225 (ERKCCVE; Table 7, Eu numbering) in the IgG2 upper hinge were substituted with residues 216-225 of the IgG1 subclass (EPKSCDKTHT; Eu numbering), and additionally the residues 233-237 (PV-AG) in the lower IgG2 hinge were substituted with residues 233-237 (EFLGG) from the IgG4 subclass. The amino acid sequence is shown in SEQ ID NO: 190 and is encoded by the codon-optimised DNA with the sequence shown as SEQ ID NO: 191.

The isatuximab-IgG2-M3-H429F mAb (Isa-IgG2-M3-H429F) used in the example comprised the CD38 specific heavy chain polypeptide of Isa-IgG2-M3 mAb (Isa-IgG2-M3) with a substitution of histidine with phenylalanine at position 429 in the CH3 region (H429F; Eu numbering).

The amino acid sequence is shown in SEQ ID NO: 204 and is encoded by the codon-optimised DNA with the sequence shown as SEQ ID NO: 205.

The isatuximab-IgG2-EFLGG mAb (Isa-IgG2-EFLGG) used in the example comprised the CD38 specific heavy chain polypeptide of Isa-IgG2-WT with the following mutation: the residues at position 233-237 of IgG2 (PV-AG) in the lower hinge were substituted with the amino acids (EFLGG) from the lower hinge of the human IgG4 subclass. The amino acid sequence is shown in SEQ ID NO: 194 and is encoded by the codon-optimised DNA with the sequence shown as SEQ ID NO: 195.

The isatuximab-IgG2-EFLGG-H429F mAb (Isa-IgG2-EFLGG-H429F) used in the example comprised the CD38 specific heavy chain polypeptide of Isa-IgG2-EFLGG with a substitution of histidine with phenylalanine at position 429 in the CH3 region (H429F; Eu numbering). The amino acid sequence is shown in SEQ ID NO: 206 and is encoded by the codon-optimised DNA with the sequence shown as SEQ ID NO: 207.

The anti-CD20 rituximab-IgG1-WT mAb (RIT-IgG1-WT) used in this example carried the V domain of rituximab. The RIT-IgG1-WT light chain polypeptide comprised of the amino acid sequence as shown in SEQ ID NO: 30 (comprising the CD20-specific VL domain of the rituximab mAb fused to human kappa constant domain) and is encoded by the codon-optimised DNA with the sequence shown as SEQ ID NO: 31. The RIT-IgG1-WT heavy chain polypeptide comprised, in the order of the N-terminus to the C-terminus, the CD20-specific VH domain of rituximab fused to the CH1-hinge-CH2-CH3 domains of human IgG1, as shown in SEQ ID NO: 28, and is encoded by the codon-optimised DNA with the sequence shown as SEQ ID NO: 29.

### Antibody Expression Vectors

The CD38 mAbs were produced using the antibody expression vector pcDNA3.4, using manufactures instructions. Briefly, antibody expression vectors consisted of a synthetic polynucleotide sequence, encoding the antibody heavy chain or light chain, appropriately placed within pcDNA3.4 (Thermo Fisher Scientific).

### Expression of Antibodies by Expi293F Cells.

Expression of IgG antibodies was conducted using transient transfection of Expi293F cells (Thermo Fisher Scientific). Expi293F cells were cultured in Expi293 Expression Medium (Life Technologies) for both cell maintenance and protein production. Transfection of the cells was performed at RT using the Life Technologies Expifectamine Transfection Kit according to the manufacturer's instructions. Briefly, cells were transfected simultaneously with IgG heavy chain plasmid (15 μg) and light chain plasmid (15 μg) diluted in Opti-MEM (Gibco) and were then cultured at 37 °C for 16 to 18 hours. After this, 150 μL of the manufacturer's Enhancer 1 and 1.5 mL of Enhancer 2 were added and the cells cultured for a further five days at 34 °C Culture supernatants were then clarified by centrifugation at 3000×g for 40 minutes, and the supernatant filtered with a 0.2 μm high flow filter (Sartorius) prior to purification. The presence of expected antibody in the supernatant was confirmed by SDS-PAGE.

Protein A Affinity Purification of mAbs.

The mAbs were purified from the supernatant of the transfected Expi293F cells by protein A affinity chromatography. Briefly, Hi-trap™ Protein A high-performance columns (GE Healthcare Life Sciences) were washed and equilibrated in binding buffer (20 mM NaH₂PO₄ pH 7.0). Clarified cell culture supernatant was loaded, the columns washed with binding buffer to baseline OD280 nm, and bound antibody was eluted with 0.1M sodium citrate tribasic dihydrate (pH 3.5) and the eluate collected in 1 mL fractions. Fractions were neutralised immediately with 1M Tris-HCl pH 9.0 and fractions containing antibody were pooled.

Size Exclusion Chromatography (SEC) of mAbs.

Following protein A affinity purification, size exclusion chromatography (SEC) was used to further purify and characterise the antibodies. The protein A purified antibodies were concentrated (typically within a concentration range of 5-10 μg/mL) using a 30 kDa molecular weight cut-off centrifuge concentrator device (Merck-Millipore). Superose 6 Increase 10/300GL columns (GE Healthcare Life Sciences) were equilibrated in PBS pH 7.2, then the concentrated protein A affinity purified antibody was loaded and separated at a flow rate of 0.5 m/min in PBS. Fractions of 0.5 mL were collected and monomeric IgG peak fractions were pooled for subsequent use. Purified antibodies were confirmed by SDS-PAGE analysis.

### Evaluation of Antigen Binding by Monoclonal Antibodies.

Purified antibodies were tested for recognition of target antigen (CD38) by flow cytometry as previously described in Trist et al., 2014 supra. The anti-CD38 mAbs were tested for binding on Raji lymphoma cells expressing CD38. Briefly, mAbs were titrated by serial 3-fold dilution, starting at a concentration of 5 μg/mL in 25 μL of FACS buffer (PBS with 0.5% (w/v) BSA). Twenty-five microliters of cells at a concentration of 5×10⁶ cells/mL were then added to the titrated mAb and incubated for 30 minutes on ice. Cells were then washed twice in FACS buffer following centrifugation (200×g for 5 minutes at 4 °C), resuspended in 50 μL of FITC conjugated F(ab′)₂ fragments of goat anti-human IgG Fc, and incubated for 30 minutes on ice in the dark. Cells were next washed twice in cold FACS buffer, resuspended in 50 μL of 1% paraformaldehyde in PBS, and incubated for 10 minutes at RT in the dark. Cells were then washed once in FACS buffer and resuspended in 200 μL FACS buffer to be analyzed by flow cytometry using a BD FACSCanto™ II flow cytometer.

### Competitive Binding Assay to Assess Relative Binding by Monoclonal Antibodies.

The binding of purified mAbs to CD38 antigen was also determined using a competitive binding assay that detects the binding of mAb to a target antigen by competition with a biotinylated IgG1 antibody against the same target epitope. Briefly, mAbs were titrated by serial 3-fold dilution, starting at a concentration of 10 μg/mL, and were incubated with Raji cells on ice for 30 minutes in L15 buffer (Lebowitz-15 medium containing 0.5% BSA). Cells were centrifuged (200×g for 5 minutes at 4 °C) and unbound antibody was washed away in L15 buffer before 5 μg/mL of biotinylated Isa-IgG1-WT was added for a further 30 minutes on ice. Cells were centrifuged and washed twice in cold PBS to remove traces of biotin that may be present in the cell culture media or L15-buffer, resuspended with 50 μL of FITC-conjugated streptavidin (BD Pharmingen) diluted in PBS, and incubated for 30 minutes on ice. Cells were then washed in cold PBS resuspended in 50 μL of 1% paraformaldehyde in PBS and incubated for 10 minutes at RT in the dark. Cells were then centrifuged and washed once in L15 buffer and resuspended in 200 μL L15 buffer to be analysed by flow cytometry using a BD FACSCanto™ II flow cytometer.

Biotinylation of Isa-IgG1-WT (1-10 mg/mL, 1 mL) was performed by the addition of 27 μL of 10 mM EZ-Link™ Sulfo-NHS-LC-Biotin (Thermo Scientific; sulfosuccinimidyl-6-[biotin-amido]hexanoate) to 1 mL of mAb at a concentration of 1-10 mg/mL in PBS, followed by incubation at room temperature. Unbound biotinylation reagent was removed by dialysis against PBS pH 7.4 and antibody concentration was determined by an absorbance at OD280 nm of 1.4 being equivalent to 1 mg/mL.

### Complement Dependent Cytotoxicity of Cells.

Cell killing (CDC) in the presence of normal human serum as a source of complement was measured by flow cytometry using the Zombie Green Fixable Viability kit (BioLegend) following opsonisation of target cells with mAb. The mAbs were titrated by serial 3-fold dilution, starting at a concentration of 5 μg/mL in 25 μL of L15 buffer. Twenty-five microliters of target cells at a concentration of 5×10⁶ cells/mL were then added to the titrated mAb and incubated for 30 minutes on ice. Cells were then centrifuged (200×g for 5 minutes at 4 °C) and washed twice in L15 buffer and resuspended in 50 μL of normal human serum that was thawed and diluted 1:3 in L15 buffer immediately before use, and incubated for 30 minutes at 37 °C Following the incubation, the cells were centrifuged and washed twice in cold PBS then resuspended in 50 μL Zombie Green (prepared in DMSO according to the manufacturer's instructions) diluted 1:500 in PBS and incubated for a further 30 minutes on ice, protected from light. Cells were then centrifuged and washed once in PBS, resuspended in 50 μL of 1% paraformaldehyde in PBS, and incubated for 10 minutes at RT in the dark. Cells were then centrifuged and washed once in L15 buffer and resuspended in 200 μL L15 buffer to be analyzed by flow cytometry using a BD FACSCanto™ II flow cytometer. Non-viable cells were identified by staining with Zombie Green Fixable Viability dye that stains the primary amines of intracellular proteins in non-viable cells with compromised membranes.

Detection of Bound C1 to mAb Opsonized Target Cells with an Anti-C1_{q} Detection Antibody.

C1 binding to antibody-opsonised cells was evaluated by flow cytometry. As Raji cells express surface receptors for C1q, Ramos lymphoma cells were used for this assay (5×10⁶ cells/mL in in Dulbecco's phosphate-buffered saline (DPBS)+0.5% BSA) and were incubated with 5 μg/mL of mAb on ice for 30 minutes. Cells were then centrifuged (200×g for 5 minutes at 4 °C) and washed twice in DPBS+0.5% BSA before they were resuspended in normal human serum diluted 1 in 3 in DPBS+0.5% BSA and incubated on ice for a further 30 minutes. The cells were then centrifuged and washed once in cold DPBS+0.5% BSA, resuspended in polyclonal-rabbit anti-C1q antiserum (Burnet Institute) diluted 1:100 in DPBS+0.5% BSA, and incubated for a further 30 minutes on ice. Cells were then centrifuged and washed once in cold DPBS+0.5% BSA, resuspended in 50 μL of APC-conjugated donkey antiserum detecting rabbit IgG (H+L) (Jackson ImmunoResearch), and incubated for a further 30 minutes on ice, protected from light. Cells were then centrifuged and washed once in DPBS, resuspended in 50 μL of 1% paraformaldehyde in PBS, and incubated for 10 minutes at RT in the dark. Cells were then washed once in DPBS+0.5% BSA and resuspended in 200 μL DPBS+0.5% BSA+1 mM EDTA to be analyzed by flow cytometry using a BD FACSCanto™ II flow cytometer. The amount of C1 bound to the mAb opsonised target cells was determined by degree of anti-C1q detected with the fluorescent labelled secondary antibody, and is represented as the median fluorescence intensity (MFI).

### Results and Discussion

Quantitation of Target Cell Binding by Hinge-Region Modified IgG2 Isatuximab mAbs with the H429F Mutation.

Isatuximab-WT and modified mAbs were produced as described above and purified sequentially by protein A affinity chromatography followed by size exclusion chromatography (SEC). SEC elution profiles demonstrated that all parental and H429F mutant mAbs contained a single monomeric IgG (H₂L₂) species with minimal oligomeric species (FIG. 72) as shown, for example, Isa-IgG2-WT compared to Isa-IgG2-H429F (FIG. 72B) or Isa-IgG2-M3 compared to Isa-IgG2-M3-H429F (FIG. 72F). The fractions containing monomeric IgG were pooled and the purified H429F mutant mAbs were analysed by SDS-PAGE (FIG. 73). This revealed the expected ~150 kDa H₂L₂ as the predominant species under non-reducing conditions. Thus, all the molecules formed disulfides between heavy chain and heavy chain (H-H), and between heavy chain and light chain (H-L). The absence of free light chain confirms the disulfide bonding patterns for the M1, M2 and M3 mutants indicated in FIG. 65. All of the antibodies, upon disulfide bond reduction with dithiothreitol, resolved to the expected ~50 kDa heavy chain and ~25 kDa light chain species. SDS-PAGE analysis of the equivalent mAbs that do not contain the H429F mutation is shown in the previous example in FIG. 67.

The purified anti-CD38 mAbs were tested by flow cytometry for direct binding to CD38-expressing Raji cells. The binding of each mAb formatted with or without the H429F mutation was first determined via detection of bound mAb with a fluorescently labelled anti-Fc antibody. In all cases, mAb binding was detected, but the binding of each of the mAbs containing the H429F mutation appeared to be reduced compared to their equivalent mAb without the H429F mutation (FIG. 74). Thus, for example, target binding of Isa-IgG2-M2-H429F was significantly lower (p<0.0001) than that of Isa-IgG2-M2 that retains histidine at position 429 (FIG. 74D). This apparent reduction in CD38 binding of the antibodies that contain the H429F mutation may reflect the process of on-target oligomerisation that is facilitated by the H429F mutation. Thus, the oligomerisation of these IgG molecules may obscure or block some epitopes in the Fc that are detected by the polyclonal anti-Fc antibody and thereby reduce binding of the fluorescent-conjugated anti-Fc antibody.

To more fully address the capacity of the mAbs to bind CD38, a competition assay was also used whereby the binding of each mAb is determined by its ability to compete with anti-CD38 mAb formatted with a wildtype H chain of human IgG1 (Isa-IgG1-WT) that has also been labelled with biotin (Isa-IgG1-WT-biotin). Such competition assays avoid any effect on the test agent of labelling or detection with a secondary reagent as may have been observed with fluorescent-conjugated anti-Fc antibody in the previous example. Isa-IgG1-H429F showed a small but significantly improved ability to compete for binding compared to Isa-IgG1-WT that retains the normal histidine at position 429 (p<0.003) (FIG. 75A). Isa-IgG2-WT was a much poorer competitor that Isa-IgG1-WT (compare FIG. 75B with 75A). Isa-IgG2-H429F showed a significantly improved ability to compete for binding compared to Isa-IgG2-WT that retains the normal histidine at position 429 (p<0.0001) (FIG. 75B). One possibility for this result is that the enhanced Fc:Fc associations conferred by the H429F mutation stabilises the bound Isa-IgG2-H429F, particularly that which forms hexamers, and results is slower dissociation of the H429F modified mAb from the cell. Thus, Isa-IgG2-H429F is a better competitor for binding to cell surface CD38 than Isa-IgG2-WT. Thus, Isa-IgG2-H429F is a binder to cell surface CD38 than Isa-IgG2-WT. In each other case Isa-IgG2-EFLGG versus Isa-IgG2-EFLGG-H429F (FIG. 75C), Isa-IgG2-M1 versus Isa-IgG2-M1-H429F (FIG. 75D), Isa-IgG2-M2 versus Isa-IgG2-M2-H429F (FIG. 75E), Isa-IgG2-M3 versus Isa-IgG2-M3-H429F (FIG. 75F), the H429F modified antibody was a better competitor for binding to cell surface CD38 than its H429 equivalent mAb. The results from this assay suggest that H429F mutant mAbs are better competitors, and so binders, than the corresponding parental mAb that retains the normal histidine at position 429. Overall, the results from this assay indicated that H429F-mutant mAbs bind to target Raji cells to better than the corresponding mAb without the H429F mutation based on their ability to compete with biotinylated Isa-IgG1-WT (FIG. 75).

H429F Mutation Enhances the Complement Dependent Killing of Target Cells by Hinge-Region Modified Isatuximab IgG2 mAbs.

The ability of each anti-CD38 mAb to mediate the killing of cells was assessed using a CDC assay. Results indicated that the H429F mutation enhanced the killing potency of anti-CD38 mAbs with an IgG1 heavy chain (FIG. 76A), an IgG2 heavy chain (FIG. 76B), and additionally an IgG2 heavy chain with upper and/or lower hinge mutations (FIG. 76D-F). The cytotoxic effect was enhanced, but to a lesser extent, by the H429F substitution in IgG2 mAb comprising only the lower hinge mutation (Isa-IgG2-EFLGG) (FIG. 76C). These results indicate that improved killing potency can be achieved by H429F modification of IgG1 and IgG2 mAbs, as well as IgG2 mAbs containing mutations in the upper and/or lower hinge region. Lastly, the presence of normal human serum (+C′) was required for the observed killing of target cells by all mAbs, either with the H429F mutation or with normal histidine at position 429, as controls lacking serum (−C′) failed to show equivalent levels of killing activity (FIG. 77).

H429F Mutation Enhances the Binding of C1 to Target Cells Opsonised with Hinge Region Modified IgG2 Isatuximab mAbs.

C1 binding following opsonisation of CD38-expressing Ramos cells by anti-CD38 mAbs was evaluated by flow cytometry using an anti-C1q antibody for detection of bound C1 complex (FIG. 78). The measured C1 binding by hinge region modified IgG2 mAbs (Isa-IgG2-M1, Isa-IgG2-M2, and Isa-IgG2-M3) was comparable to Isa-IgG2-EFLGG, suggesting that the incorporation of the permissive lower hinge residues from IgG4 (EFLGG) may provide improved C1 binding capabilities.

Moreover, C1 binding was greatly enhanced for cells treated with H429F mutant mAbs compared to the corresponding parental mAb. For example, C1q binding was enhanced 7.3-fold when H429F was introduced into IgG2 (MFI of Isa-IgG2-WT: 2402, MFI of Isa-IgG2-H429F: 17554). Similarly, C1 binding was enhanced 6.8-fold when H429F was introduced into the IgG2 M2 mutant (MFI of Isa-IgG2-M2: 9145, MFI of Isa-IgG2-M2-H429F: 62480). Thus, the H429F substitution improves C1 binding in all mAbs tested, including IgG1, IgG2, and IgG2 with mutations in the upper and/or lower hinge regions. This is consistent with the enhanced capacity of H429F mutant mAbs to form on-target oligomers (particularly hexamers) that can provide the optimal configuration for the binding of the hexameric C1 complex, thereby triggering the activation of the classical complement pathway that leads to cell lysis via CDC.

### Conclusion

The data obtained in this example demonstrates that the functional effects arising from H429F modification, such as enhanced C1 binding and CDC activity, can be applied to the IgG2 subclass as well as IgG2 antibodies engineered in the upper and lower hinge region that provide improvements in the binding of mAb to surface antigen, as well as C1 binding and subsequent target cell killing.

### Example 21Activity Analysis of H429F Modification in Anti-CD38 IgG1 and IgG2 mAbs with Mutations in the CH2 Domain

### Introduction

In this example, the effect of the H429F mutation was further investigated in anti-CD38 IgG1 and IgG2 mAbs comprising additional mutations in the CH2 domain that may alter the capacity of mAbs to activate immune effector functions, such as FcγR dependent and serum complement dependent responses. These mutations include, LALA (L234A and L235A; Eu numbering), in the lower hinge region that is known to impair interactions with all FcγRs and complement component C1q when introduced into human IgG1 (Chenoweth et al., supra 2020; Wang et al., 2018 supra) and murine IgG2a (Lo et al., J Biol Chem 292(9):3900-3908, 2017). Other mutations such as mutation of residue 274 in the CH2 domain of human IgG, that exists as a lysine (K274) in IgG1 and a glutamine (Q274) in IgG2, IgG3, and IgG4. Additionally, as species specific differences may be exploited to design mAbs with altered C1q binding capabilities, an additional mutant was also generated that comprised of two mutations, H268E and A327D, in the IgG1 CH2 domain that exist in mouse and rabbit IgG (E268) and mouse IgG2a (D327) in addition to H429F. This mutant was tested to examine whether the inclusion of these CH2 residues had an additive effect on enhancing H429F-induced CDC activity.

Importantly, these mutations in the CH2 domain and lower hinge are distant from the H429 residue in the CH3 domain. As IgG heavy chains are modular in nature, it is possible that mutation in one domain may have no effect upon the functional consequences of mutation in a different domain. However, pairwise combinations of mutations should always be assessed, especially when both mutations impact the same biological function. Two mutants, or combination of two classes of mutation, may have an additive or enhancing effect or may act in an opposing manner on function. For instance, the H429F mutation enhances CDC potency through the process of on-target hexamerisation, whilst the LALA mutation reduces CDC activity by inhibiting interactions with C1q. Therefore, it is important to assess whether H429F mutation overrides the functional effect of the LALA mutation, and the degree of mAb-induced complement dependent target lysis that results.

In this example, the interaction of H429F with these mutations in a different antibody domain, and subsequent effect upon target cell killing activity, is explored.

### Methods and Materials

### Antibodies and Antibody Constructs.

All anti-CD38 mAbs used in this example carried the V domain of isatuximab, that comprised a light chain polypeptide with the amino acid sequence shown as SEQ ID NO: 44 (comprising the CD38-specific VL domain of the isatuximab mAb fused to human kappa constant domain) that is encoded by the codon-optimised DNA with the sequence shown as SEQ ID NO: 45.

The heavy chain sequences of the mAbs used in this example are described below and listed as sequence IDs in Table 12 below. Modified mAb heavy chains were produced by the synthesis of synthetic cDNA sequences encoding the antibody heavy chain with the relevant mutations using standard molecular biology techniques.

TABLE 12
Anti-CD38 mAbs using human IgG1 or IgG2 heavy chains and mutant heavy chains

| | | | Region/Amino Acid position(s) | | | | | | | |
|---|---|---|---|---|---|---|---|---|---|---|
| mAb HC | Protein | DNA | CH1/ | UH/ | UH/ | LH/ | CH1/ | CH2/ | CH2/ | CH3/ |
| Name | Seq ID | Seq ID | 131 | 219 | 220 | 233-237 | 131 | 274 | 327 | 429 |
| Isa-IgG1- | 42 | 43 | S | S | C | ELLGG | H | K | A | H |
| WT | | | | | | | | | | |
| Isa-IgG1- | 196 | 197 | S | S | C | ELLGG | H | K | A | F |
| H429F | | | | | | | | | | |
| Isa-IgG1- | 208 | 209 | S | S | C | EAAGG | H | K | A | H |
| LALA | | | | | | | | | | |
| Isa-IgG1- | 210 | 211 | S | S | C | EAAGG | H | K | A | F |
| LALA- | | | | | | | | | | |
| H429F | | | | | | | | | | |
| Isa-IgG1- | 212 | 213 | S | S | C | ELLGG | H | Q | A | H |
| K274Q | | | | | | | | | | |
| Isa-IgG1- | 214 | 215 | S | S | C | ELLGG | H | Q | A | F |
| K274Q- | | | | | | | | | | |
| H429F | | | | | | | | | | |
| Isa-IgG1- | 216 | 217 | S | S | C | ELLGG | E | K | D | F |
| H268E- | | | | | | | | | | |
| A327D- | | | | | | | | | | |
| H429F | | | | | | | | | | |
| Isa-IgG2- | 184 | 185 | C | C | C | PV-AG | H | Q | A | H |
| WT | | | | | | | | | | |
| Isa-IgG2- | 198 | 199 | C | C | C | PV-AG | H | Q | A | F |
| H429F | | | | | | | | | | |
| Isa-IgG2- | 218 | 219 | C | C | C | EAAGG | H | Q | G | F |
| LALA- | | | | | | | | | | |
| H429F | | | | | | | | | | |
| Isa-IgG2- | 220 | 221 | C | C | C | PV-AG | H | K | G | F |
| Q274K- | | | | | | | | | | |
| H429F | | | | | | | | | | |

HC = heavy chain, CH1 = constant domain 1 of the HC, UH = upper hinge, LH = lower hinge (N-terminus of the CH2 domain).
All amino acid positions are numbered according to the Eu numbering system that aligns heavy chain residues to that of a wildtype human IgGI wildtype sequence, as shown in FIG. 3 and FIG. 4 (Table 7 and Table 9).

The isatuximab-IgG1-WT mAb (Isa-IgG1-WT) used in the example comprised a CD38 specific heavy chain polypeptide comprising, in the order of the N-terminus to the C-terminus, the CD38-specific VH domain of isatuximab fused to the CH1-hinge-CH2-CH3 domains of human IgG1 (SEQ ID NO: 42), and is encoded by the codon-optimised DNA with the sequence shown as SEQ ID NO: 43.

The isatuximab-IgG1-H429F mAb (Isa-IgG1-H429F) used in the example comprised a CD38 specific heavy chain polypeptide comprising, in the order of the N-terminus to the C-terminus, the CD38-specific VH domain of isatuximab fused to the CH1-hinge-CH2-CH3 domains of human IgG1 with a substitution of histidine with phenylalanine at position 429 (H429F; Eu numbering) in the CH3 region (SEQ ID NO: 196), and is encoded by the codon-optimised DNA with the sequence shown as SEQ ID NO: 197.

The isatuximab-IgG1-LALA mAb (Isa-IgG1-LALA) used in the example comprised a CD38 specific heavy chain polypeptide comprising, in the order of the N-terminus to the C-terminus, the CD38-specific VH domain of isatuximab fused to the CH1-hinge-CH2-CH3 domains of human IgG1 with a substitution of alanine in place of leucine at positions 234 and 235 in the lower hinge (L234A and L235A; Eu numbering) (SEQ ID NO: 208), and is encoded by the codon-optimised DNA with the sequence shown as SEQ ID NO: 209.

The isatuximab-IgG1-LALA-H429F mAb (Isa-IgG1-LALA-H429F) used in the example comprised a CD38 specific heavy chain polypeptide comprising, in the order of the N-terminus to the C-terminus, the CD38-specific VH domain of isatuximab fused to the CH1-hinge-CH2-CH3 domains of human IgG1 with a substitution of alanine in place of leucine at positions 234 and 235 in the lower hinge (L234A and L235A; Eu numbering), as well as substitution of histidine with phenylalanine at position 429 (H429F; Eu numbering) in the CH3 region (SEQ ID NO: 210), and is encoded by the codon-optimised DNA with the sequence shown as SEQ ID NO: 211.

The isatuximab-IgG1-K274Q mAb (Isa-IgG1-K274Q) used in the example comprised a CD38 specific heavy chain polypeptide comprising, in the order of the N-terminus to the C-terminus, the CD38-specific VH domain of isatuximab fused to the CH1-hinge-CH2-CH3 domains of human IgG1 with a substitution of glutamine in place of lysine at position 274 in the CH2 domain (K274Q; Eu numbering) (SEQ ID NO: 212), and is encoded by the codon-optimised DNA with the sequence shown as SEQ ID NO: 213.

The isatuximab-IgG1-K274Q-H429F mAb (Isa-IgG1-K274Q-H429F) used in the example comprised a CD38 specific heavy chain polypeptide comprising, in the order of the N-terminus to the C-terminus, the CD38-specific VH domain of isatuximab fused to the CH1-hinge-CH2-CH3 domains of human IgG1 with a substitution of glutamine in place of lysine at position 274 in the CH2 domain (K274Q; Eu numbering), as well as substitution of histidine with phenylalanine at position 429 (H429F; Eu numbering) in the CH3 region (SEQ ID NO: 214), and is encoded by the codon-optimised DNA with the sequence shown as SEQ ID NO: 215.

The isatuximab-IgG1-H268E-A327D-H429F mAb (Isa-IgG1-H268E-A327D-H429F) used in the example comprised a CD38 specific heavy chain polypeptide comprising, in the order of the N-terminus to the C-terminus, the CD38-specific VH domain of isatuximab fused to the CH1-hinge-CH2-CH3 domains of human IgG1 with a substitution of glutamic acid in place of histidine at position 268 in the CH2 domain (H268E; Eu numbering), a substitution of aspartic acid in place of alanine at position 327 in the CH2 domain (A327D; Eu numbering), as well as substitution of histidine with phenylalanine at position 429 (H429F; Eu numbering) in the CH3 region (SEQ ID NO: 216), and is encoded by the codon-optimised DNA with the sequence shown as SEQ ID NO: 217.

The isatuximab-IgG2-WT mAb (Isa-IgG2-WT) used in the example comprised a CD38 specific heavy chain polypeptide comprising, in the order of the N-terminus to the C-terminus, the CD38-specific VH domain of isatuximab fused to the CH1-hinge-CH2-CH3 domains of human IgG2 (SEQ ID NO: 184), and is encoded by the codon-optimised DNA with the sequence shown as SEQ ID NO: 185.

The isatuximab-IgG2-H429F mAb (Isa-IgG2-H429F) used in the example comprised a CD38 specific heavy chain polypeptide comprising, in the order of the N-terminus to the C-terminus, the CD38-specific VH domain of isatuximab fused to the CH1-hinge-CH2-CH3 domains of human IgG2 with a substitution of histidine with phenylalanine at position 429 (H429F; Eu numbering) in the CH3 region (SEQ ID NO: 198), and is encoded by the codon-optimised DNA with the sequence shown as SEQ ID NO: 199.

The isatuximab-IgG2-LALA-H429F mAb (Isa-IgG2-LALA-H429F) used in the example comprised the CD38 specific heavy chain polypeptide of Isa-IgG2-WT modified with a substitution of alanine in place of leucine at positions 234 and 235 in the lower hinge (L234A and L235A; Eu numbering), as well as substitution of histidine with phenylalanine at position 429 (H429F; Eu numbering) in the CH3 region, as shown in SEQ ID NO: 218, and is encoded by the codon-optimised DNA with the sequence shown as SEQ ID NO: 219.

The isatuximab-IgG2-Q274K-H429F mAb (Isa-IgG2-Q274K-H429F) used in the example comprised the CD38 specific heavy chain polypeptide of Isa-IgG2-WT modified with a substitution of lysine in place of glutamine at position 274 in the CH2 domain (Q274K; Eu numbering), as well as substitution of histidine with phenylalanine at position 429 (H429F; Eu numbering) in the CH3 region, as shown in SEQ ID: 220, and is encoded by the codon-optimised DNA with the sequence shown as SEQ ID NO: 221.

### Expression of Antibodies by Expi293F Cells.

Expression of IgG antibodies was conducted using transient transfection of Expi293F cells (Thermo Fisher Scientific). Expi293F cells were cultured in Expi293 Expression Medium (Life Technologies) for both maintenance and protein production. Transfection of the cells was performed at RT using the Life Technologies Expifectamine Transfection Kit according to the manufacturer's instructions. Briefly, cells were transfected simultaneously with IgG heavy chain plasmid (15 μg) and light chain plasmid (15 μg) diluted in Opti-MEM (Gibco) and were then cultured at 37 °C for 16 to 18 hours. After this, 150 μL of the manufacturer's Enhancer 1 and 1.5 ml of Enhancer 2 were added and the cells cultured for a further five days at 34 °C Culture supernatants were then clarified by centrifugation at 3000×g for 40 minutes, and the supernatant filtered with a 0.2 μm high flow filter (Sartorius) prior to purification. The presence of expected antibody in the supernatant was confirmed by SDS-PAGE.

Protein a Affinity Purification of mAbs.

The mAbs were purified from the supernatant of the transfected Expi293F cells by protein A affinity chromatography. Briefly, Hi-trap™ Protein A high-performance columns (GE Healthcare Life Sciences) were washed and equilibrated in binding buffer (20 mM NaH₂PO₄ pH 7.0). Clarified cell culture supernatant was loaded, the columns washed with binding buffer to baseline OD280 nm, and bound antibody was eluted with 0.1M sodium citrate tribasic dihydrate (pH 3.5) and the eluate collected in 1 mL fractions. Fractions were neutralised immediately with 1M Tris-HCl pH 9.0 and fractions containing antibody were pooled.

Size Exclusion Chromatography (SEC) of mAbs.

Following protein A affinity purification, size exclusion chromatography (SEC) was used to further purify and characterise the antibodies. The protein A purified antibodies were concentrated (typically within a concentration range of 5-10 μg/mL) using a 30 kDa molecular weight cut-off centrifuge concentrator device (Merck-Millipore). Superose 6 Increase 10/300GL columns (GE Healthcare Life Sciences) were equilibrated in PBS pH 7.2, then the concentrated protein A affinity purified antibody was loaded and separated at a flow rate of 0.5 m/min in PBS. Fractions of 0.5 mL were collected and monomeric IgG peak fractions were pooled for subsequent use. Purified antibodies were confirmed by SDS-PAGE analysis.

### Evaluation of Antigen Binding by Monoclonal Antibodies.

Purified antibodies were tested for recognition of target antigen (CD38) by flow cytometry as previously described in Trist et al., 2014 supra. The anti-CD38 mAbs were tested for binding on Raji lymphoma cells expressing CD38. Briefly, mAbs were titrated by serial 3-fold dilution, starting at a concentration of 5 μg/mL in 25 μL of FACS buffer (PBS with 0.5% (w/v) BSA). Twenty-five microliters of cells at a concentration of 5×10⁶ cells/mL were then added to the titrated mAb and incubated for 30 minutes on ice. Cells were then washed twice in FACS buffer following centrifugation (200×g for 5 minutes at 4 °C), resuspended with 50 μL of FITC conjugated F(ab′)₂ fragments of goat anti-human IgG Fc, and incubated for 30 minutes on ice in the dark. Cells were next washed twice in cold FACS buffer, resuspended in 50 μL of 1% paraformaldehyde in PBS, and incubated for 10 minutes at RT in the dark. Cells were then washed once in FACS buffer and resuspended in 200 μL FACS buffer to be analyzed by flow cytometry using a BD FACSCanto™ II flow cytometer.

### Competitive Binding Assay to Assess Relative Binding by Monoclonal Antibodies.

The binding of purified mAbs to CD38 antigen was also determined using a competitive binding assay that detects the binding of mAb to a target CD38 by competition with biotinylated Isa-IgG1-WT against the same target epitope. Briefly, mAbs were titrated by serial 3-fold dilution, starting at a concentration of 10 μg/mL, and were incubated with Raji cells on ice for 30 minutes in L15 buffer (Lebowitz-15 medium containing 0.5% BSA). Cells were centrifuged (200×g for 5 minutes at 4 °C) and unbound antibody was washed away in L15 buffer before 5 μg/mL of biotinylated Isa-IgG1-WT was added for a further 30 minutes on ice. Cells were centrifuged and washed twice in cold PBS to remove traces of biotin that may be present in the cell culture media or L15-buffer, resuspended with 50 μL of FITC-conjugated streptavidin (BD Pharmingen) diluted in PBS, and incubated for 30 minutes on ice. Cells were then washed in cold PBS resuspended in 50 μL of 1% paraformaldehyde in PBS and incubated for 10 minutes at RT in the dark. Cells were then centrifuged and washed once in L15 buffer and resuspended in 200 μL L15 buffer to be analysed by flow cytometry using a BD FACSCanto™ II flow cytometer.

Biotinylation of Isa-IgG1-WT (1-10 mg/mL, 1 mL) was performed by the addition of 27 μL of 10 mM EZ-Link™ Sulfo-NHS-LC-Biotin (Thermo Scientific; sulfosuccinimidyl-6-[biotin-amido]hexanoate) to 1 mL of mAb at a concentration of 1-10 mg/mL in PBS, followed by incubation at room temperature. Unbound biotinylation reagent was removed by dialysis against PBS pH 7.4 and antibody concentration was determined by an absorbance at 280 nm of 1.4 being equivalent to 1 mg/mL.

### Complement Dependent Lysis of Cells.

CDC was measured by flow cytometry using the Zombie Green Fixable Viability kit (BioLegend) following opsonisation of target cells with mAb. MAbs were titrated by serial 3-fold dilution, starting at a concentration of 5 μg/mL in 25 μL of L15 buffer. Twenty-five microliters of target cells at a concentration of 5×10⁶ cells/ml were then added to the titrated mAb and incubated for 30 minutes on ice. Cells were then centrifuged (200×g for 5 minutes at 4 °C) and washed twice in L15 buffer and resuspended in 50 μL of normal human serum that was thawed and diluted 1:3 in L15 buffer immediately before use, and incubated for 30 minutes at 37 °C Following the incubation, the cells were centrifuged and washed twice in cold PBS then resuspended in 50 μL Zombie Green (prepared in DMSO according to the manufacturer's instructions; BioLegend) diluted 1:500 in PBS and incubated for a further 30 mins on ice, protected from light. Cells were then centrifuged and washed once in PBS, resuspended in 50 μL of 1% paraformaldehyde in PBS, and incubated for 10 minutes at RT in the dark. Cells were then centrifuged and washed once in L15 buffer and resuspended in 200 μL L15 buffer to be analyzed by flow cytometry using a BD FACSCanto™ II flow cytometer.

### Detection of C1q Binding to mAb Opsonized CD38-Expressing Target Cells.

C1q binding to antibody-opsonised cells was evaluated by flow cytometry. Ramos lymphoma cells (5×10⁶ cells/ml in FACS buffer with 1 mM EDTA) were incubated with 5 μg/mL of mAb on ice for 30 minutes. Cells were then centrifuged (200×g for 5 minutes at 4 °C) and washed twice in FACS buffer, resuspended in FACS buffer containing 10 μg/mL of purified human C1q (Merck Millipore) and incubated on ice for a further 30 minutes. The cells were then centrifuged and washed twice in cold FACS buffer, resuspended in rabbit anti-C1q (Burnet Institute) diluted 1:100 in FACS buffer, and incubated for a further 30 minutes on ice. Cells were then centrifuged and washed twice in cold FACS buffer, resuspended in 50 μL of APC-conjugated donkey antisera detecting rabbit antibody (Jackson ImmunoResearch), and incubated for a further 30 minutes on ice, protected from light. Cells were then centrifuged and washed once in FACS buffer, resuspended in 50 μL of 1% paraformaldehyde/PBS, and incubated for 10 minutes at RT in the dark. Cells were then washed once in FACS buffer and resuspended in 200 μL FACS buffer to be analyzed by flow cytometry using a BD FACSCanto™ II flow cytometer.

### Results and Discussion

Quantitation of Target Cell Binding by Isatuximab IgG1 and IgG2 mAbs with CH2 and CH3 Domain Mutations.

The anti-CD38 IgG1 and IgG2 mAbs used in this example were generated to examine the effect of pairing the H429F mutation with mutations elsewhere within the IgG molecule that may additionally alter immune effector responses, such as the complement dependent killing of target cells. All anti-CD38 mAbs were produced as described above and purified separately by protein A affinity chromatography followed by size exclusion chromatography (SEC). The SEC elution profiles demonstrated a peak comprising of single a monomeric IgG (H₂L₂) species with minimal oligomeric species (FIG. 79). The fractions containing monomeric IgG were pooled and the purified mAbs were examined by SDS-PAGE analysis (FIG. 80). This revealed the expected ~150 kDa H₂L₂ as the predominate species under non-reducing conditions. Thus, all the molecules formed disulfides between, heavy chain and heavy chain (H-H), and between heavy chain and light chain (H-L). All the antibodies, upon disulfide bond reduction in dithiothreitol, resolved to the expected ~50 kDa heavy chain and ~25 kDa light chain species.

The purified anti-CD38 isatuximab mAbs were tested by flow cytometry for direct binding to CD38-expressing Raji cells. Quantitation of mAb binding using a fluorescently labelled anti-Fc antibody indicated similar overall binding activity between unmodified (WT) and mutant mAbs (FIG. 81). To address the capacity of mAbs to detect CD38 using an alternative method for more stringent comparison, a competition assay was also utilised whereby the binding of each mAb is determined by its ability to compete with anti-CD38 mAb formatted with a wildtype H chain of human IgG1 that has been labelled with biotin (Isa-IgG1-WT-biotin). Results from this assay indicated that all IgG1 mAbs have equivalent binding to target Raji cells (FIG. 82A, 82B), whilst two IgG2 mutants (Isa-IgG2-H429F and Isa-IgG2-Q274K-H429F) were better competitors for binding to target Raji cells compared to the unmodified Isa-IgG2-WT mAb (FIG. 82C).

The results from both the direct binding assay and competition assay demonstrated that all mAbs effectively bound to target cells.

Evaluation of the Complement Dependent Killing of Target Cells by Isatuximab IgG1 and IgG2 mAbs with CH2 and CH3 Domain Mutations.

The capacity of each anti-CD38 mAb to kill target CD38 expressing Raji cells was evaluated using a CDC assay (FIG. 83). Whilst the killing potency of the anti-CD38 IgG1 mAb comprising the H429F mutation, Isa-IgG1-H429F, was significantly (p<0.0001) enhanced compared to unmodified IgG1-WT, the combination of LALA with H429F reduced the enhanced potency conferred by the H429F mutation (FIG. 83A). It is important to note that cell killing was greater for the Isa-IgG1-LALA-H429F mutant compared to unmodified IgG1-WT, particularly at a concentration of 1.6 μg/mL or greater, suggesting that at high levels of opsonisation H429F partially restores the diminished CDC activity imposed by the LALA mutation.

The effect of other CH2 mutations on the potency of anti-CD38 IgG1 mAbs was also assessed, including the K274Q mutation either alone or in combination with H429F, as well as the triple mutant mAb comprising the H268E, A327D, and H429F mutations (FIG. 83B). Results indicated a reduction in the killing potency of IgG1 mAbs of Isa-IgG1-K274Q-H429F and Isa-IgG1-H268E-A327D-H429F compared to the H429F single mutant (Isa-IgG1-H429F). Clearly K274Q or H268E-A327D was introduced into the H chain in the mAbs impairs killing potency. The reduced potency of Isa-IgG1-K274Q-H429F compared to Isa-IgG1-H429F was surprising as this mutation, K274Q, was found not to significantly affect the binding of C1q in the context of an Fc hexamer, comprised of IgG1-Fc-fused to the tail-piece of human IgM (Rowley et al., Commun Biol 1:146, 2018). The combination of K274Q-H429F mutation in IgG1 may be a useful way of moderating complement activity.

The cell killing activity of anti-CD38 IgG2 mAbs comprising either the LALA mutation or Q274K substitution alongside H429F substitution was next examined (FIG. 83C). Isa-IgG2-LALA-H429F displayed a decrease in killing potency compared to Isa-IgG2-H429F. Surprisingly, the combination of Q274K with H429F resulted in the abolition of any detectable cell killing activity at all concentrations of mAb tested. Notably, as this mAb displays efficient binding to target Raji cells (FIG. 81C, FIG. 82C), this indicates that the Q274K mutation impairs the ability of human IgG2 to induce target cell lysis following binding to surface antigen. In the context of these IgG2-H429F formatted mAbs, the Q274K mutation more strongly inhibited cytotoxicity than the LALA mutation. Thus, the combination of Q274K-H429F mutation in IgG2 mAbs, and mAbs with modified IgG2 backbones, may be a useful way of moderating serum mediated cytotoxicity.

Lastly, the presence of human serum containing complement proteins was required for the observed cell killing activity of each mAb, as controls lacking serum failed to induce detectable cytotoxicity above background (no mAb treatment) (FIG. 84). Thus, mutations at position 274 (K274Q in IgG1 and Q274K in IgG2) may have applications in the context of normal IgG subclasses and in H429 modified IgGs, in the design of ‘complement-mediated killing inert’ therapeutic mAbs.

Evaluation of C1_{q} Binding to Cells Opsonised with Isatuximab IgG1 and IgG2 mAbs with CH2 and CH3 Domain Mutations.

C1q binding following opsonisation of CD38-expressing Ramos cells by anti-CD38 mAbs was evaluated by flow cytometry (FIG. 85). C1q binding was not detectable above background control levels for unmodified Isa-IgG1-WT as well as Isa-IgG1-LALA and Isa-IgG1-LALA-H429F that contain the LALA mutation that is known to impair interactions with C1q (Chenoweth et al., supra 2020; Wang et al., 2018 supra). However, Isa-IgG1-H429F demonstrated enhanced binding of C1q (FIG. 85A). This suggests that the LALA mutation functions to impair C1q binding either alone or in combination with H429F mutation, through its proposed mechanism of the disruption of the C1q binding site in the lower hinge region. Although C1q binding is impaired when LALA is combined with H429F, it is worthwhile noting that some C1q may still be able to bind to sufficient quantities of mAb on the target surface, as Isa-IgG1-LALA-H429F still exhibited detectable CDC activity at the higher range of mAb concentration tested, although the cytotoxic effect was reduced compared to H429F alone (FIG. 83A).

As shown in FIG. 85B, Isa-IgG1-K274Q did not show detectable levels of C1q binding, whereas the combination of K274Q with H429F (Isa-IgG1-K274Q) bound C1q, as did the IgG1 mAb comprising the H429F mutation alongside H268E and A327D (Isa-IgG1-H268E-A327D-H429F). Therefore, it appears that whilst H429F substitution endows IgG1 molecules with an enhanced capacity to bind C1q, that is consistent with the oligomerisation of mAbs on the target cell surface, the additional mutations evaluated in this example (K274Q, H268E and A327D) do not provide further enhancement of C1q binding.

Consistent with the lower CDC activity of IgG2 mAbs, C1q binding was not detectable above background control levels on Ramos cells treated with either unmodified (Isa-IgG2-WT) or H429F mutant IgG2 mAbs including Isa-IgG2-H429F, Isa-IgG2-Q274K-H429F and Isa-IgG2-LALA-H429F (FIG. 84C). Notably, IgG2 interacts poorly with C1q but effectively with the C1 complex that is stabilised by the C1r₂C1s₂ subcomponents (Zwarthoff et al., 2021 supra). This may explain the failure of this assay to detect C1q bound by IgG2 mAbs, as this utilises purified C1q rather than normal human serum that contains all of the components of serum complement including the C1 complex. Supporting this, Isa-IgG2-H429F was shown to bind C1 in FIG. 78 but failed to bind to purified C1q in the result shown here in FIG. 84C.

### Conclusion

The data obtained in this example demonstrates that mutations elsewhere in the IgG1 or IgG2 heavy chain can be paired with the H429F substitution in the CH3 domain, that in turn can give rise to differences in antibody function. Owing to the modular nature of antibodies, the enhanced oligomerisation mediated by H429F substitution in the CH3 domain can be retained whilst mutations can be introduced into other antibody domains that may affect the binding of effector molecules, such as C1q, to produce a mAb with selectively enhanced or diminished effector functions, depending on the nature of the mutations.

As shown in FIG. 83, from the panel of mutant mAbs tested in this example, those containing a single H429F mutation in the CH3 domain demonstrated the greatest enhancement in target cell killing activity. Furthermore, mAbs that comprised a H429F mutation paired with the LALA mutation that reduces C1q binding retain some ability to induce cell killing, that was particularly evident at higher concentrations of mAb treatment. This indicates that the enhanced avidity mediated by H429F substitution may enable sufficient C1q (or C1 complex) binding to partially restore target cell killing activity.

Importantly, a mutation in the CH2 domain of human IgG2 (Q274K) was found to abolish all detectable CDC activity when paired with H429F, which may have applications in the development of therapeutic mAbs seeking to avoid activation of the serum complement cascade, whilst retaining the other benefits of H429F-mediated IgG oligomerisation (such as enhanced signal amplification through target receptor clustering).

### Example 23 SARS-CoV-2 mAb S2P6

The most sequence diverse areas of immunoglobulins outside the variable domains are the hinge sequences, as seen in the alignments of amino acid sequence of the constant regions and hinges of the IgG heavy chains (FIG. 3). It should also be noted that note the N-terminus of the CH2 domain sequence also incorporates sequences that are considered structurally as part of the hinge region of the IgG protein, and which is referred to in the field and herein as the lower hinge (e.g., APELLGG of IgG1 and IgG3, APEFLGG of IgG4, and APPV-AG of IgG2). A more detailed alignment of the hinges of the human IgG subclasses is shown for clarity in Tables 7, 8 and 9 and show the hinges as comprising an upper hinge, core hinge and lower hinge.

The human IgG2 heavy chain is unique as it is the only IgG subclass wherein the upper hinge is constrained because of inter- and intra-heavy chain disulphide bonds, owing to the presence of additional cysteine residues in the IgG2 upper hinge (C219, C220 as shown in Table 7).

Additionally, the light chain can attach to the heavy chain via disulphide bonding in one of three positions—cysteine 131 in the CH1 domain, or cysteine 219 and cysteine 220 in the upper hinge.

S2P6 is a broadly neutralising human IgG1 anti-SARS-CoV-2 monoclonal antibody that detects an epitope in the S2-region of the SARS-CoV-2 spike protein. The spike protein of SARS-CoV-2 is a homo-trimeric protein expressed on the surface of the virus envelope or on the surface of virus-infected cells.

A number of mAbs were produced utilising the variable light chain and variable heavy chain domains of the S2P6 mAb. The heavy chains of the mAbs were formatted as human IgG1 or human IgG2 subclass heavy chains. These chains were either unmodified (WT) heavy chains or mutated heavy chains that contained one or more amino acid sequence changes. The heavy chain sequences of the mAbs used in this example are described below and are listed with their sequence ID Numbers in Table 13.

The killing potency of the different IgG2 mAbs was determined using CoV-2 spike protein expressing cells (Ramos-S cells) and was compared to killing by S2P6 mAb comprising IgG1 WT or mutated heavy chains.

### Methods

The mAbs produced were SARS-CoV-2 mAbs based on S2P6.

### S2P6 Light Chain

The light chain polypeptide comprising the SARS-CoV-2-specific VL domain of S2P6 was fused to human kappa light chain constant domain comprising the amino acid sequence shown as SEQ ID NO: 80 that is encoded by the codon-optimised DNA with the sequence shown as SEQ ID NO: 81.

### S2P6 IgG1 Heavy Chains

IgG1 mAbs used in this example are described in detail in Example 12. The S2P6-IgG1-WT mAb (S2P6-IgG1-WT a.k.a S2P6-WT as described in Example 12) comprised a SARS-CoV-2 specific heavy chain polypeptide comprising, in the order of the N-terminus to the C-terminus, the SARS-CoV-2-specific VH domain of S2P6 fused to the CH1-hinge-CH2-CH3 domains of human IgG1 (SEQ ID NO: 78), and is encoded by the codon-optimised DNA with the sequence shown as SEQ ID NO: 79.

The S2P6-IgG1-H429F mAb (S2P6-IgG1-H429F a.k.a S2P6-H429F) comprised a SARS-CoV-2 specific heavy chain polypeptide comprising, in the order of the N-terminus to the C-terminus, the SARS-CoV-2-specific VH domain of S2P6 fused to the CH1-hinge-CH2-CH3 domains of human IgG1 with the further substitution of histidine with phenylalanine at position 429 (H429F; Eu numbering) in the CH3 region (SEQ ID NO: 90) and is encoded by the codon-optimised DNA with the sequence shown as SEQ ID NO: 91.

### S2P6 IgG2 Heavy Chains

The S2P6-IgG2-WT mAb comprised a SARS-CoV-2 specific heavy chain polypeptide comprising, in the order of the N-terminus to the C-terminus, the SARS-CoV-2-specific VH domain of S2P6 fused to the CH1-hinge-CH2-CH3 domains of human IgG2 (SEQ ID NO: 224), and is encoded by the codon-optimised DNA with the sequence shown as SEQ ID NO: 225.

The S2P6-IgG2-H429F mAb comprised a SARS-CoV-2 specific heavy chain polypeptide comprising, in the order of the N-terminus to the C-terminus, the SARS-CoV-2-specific VH domain of S2P6 fused to the CH1-hinge-CH2-CH3 domains of human IgG2 with the further substitution of histidine with phenylalanine at position 429 (H429F; Eu numbering) in the CH3 region (SEQ ID NO: 226), and is encoded by the codon-optimised DNA with the sequence shown as SEQ ID NO: 227.

A series of additional mAbs was generated comprising mutations in one or more positions in the IgG2 H chain. Details of these mAbs are described in Example 19.

The mAbs S2P6-IgG2-M1, S2P6-IgG2-M2, S2P6-IgG2-M3 as well as mAbs S2P6-IgG2-M1-H429F, S2P6-IgG2-M2-H429F, S2P6-IgG2-M3-H429F (Table 13 and FIG. 65) were based on the human IgG2 heavy chain subclass, which was mutated such that these mAbs contained the sequence, EFLGG, being part of the lower hinge sequence of IgG4. Further mutations were introduced wherein cysteine residues were replaced with serine to unconstrain the upper hinge by removing inter- and intra-H chain disulfide bonding in the upper hinge, which is a characteristic of IgG2. Furthermore, these cysteine-to-serine mutations also provided only a single cysteine residue for light chain disulphide bonding in the upper hinge (FIG. 65). Accordingly, in all of these mAbs the cysteine at position 131 within the CH1 domain has been replaced with serine (C131S). Additionally, in mAb S2P6-IgG2-M1 and S2P6-IgG2-M1-H429F, the cysteine at position 220 is substituted with serine (C220S) and as a result only Cys119 is available for light chain bonding. In mAb S2P6-IgG2-M2 and S2P6-IgG2-M2-H429F, the cysteine at position 219 is substituted with serine (C219S) and as a result only the cysteine at position 220 is available for light chain bonding. In the mAbs S2P6-IgG2-M3 and S2P6-IgG2-M3-H429F, the upper hinge of IgG2 has been replaced with the upper hinge of IgG1 that retains a cysteine at position 220, and therefore light chain disulphide bonding occurs with this cysteine at position 220. Furthermore, the mAbs S2P6-IgG2-M1-H429F, S2P6-IgG2-M2-H429F, and S2P6-IgG2-M3-H429F also contain the hexamerising mutation H429F.

The mAbs were generated as follows:

The S2P6-IgG2-M1 mAb (a.k.a. S2P6 IgG2 C131S C220S FLGG) comprised the SARS-CoV-2 specific heavy chain polypeptide of S2P6-IgG2-WT with the following mutations: the cysteine at position 220 in the upper hinge was substituted with serine (C220S; Eu numbering) and the residues at position 233-237 of IgG2 (PV-AG) in the lower hinge were substituted with the amino acids (EFLGG; Eu numbering) from the human IgG4 subclass. The amino acid sequence is shown in SEQ ID NO: 228 and is encoded by the codon-optimised DNA with the sequence shown as SEQ ID NO: 229.

The S2P6-IgG2-M1-H429F mAb (a.k.a. S2P6 IgG2 C131S C220S FLGG H429F) comprised the SARS-CoV-2 specific heavy chain polypeptide of S2P6-IgG2-M1 mAb with the further substitution of histidine with phenylalanine at position 429 in the CH3 region (H429F; Eu numbering). The amino acid sequence is shown in SEQ ID NO: 230 and is encoded by the codon-optimised DNA with the sequence shown as SEQ ID NO: 231.

The S2P6-IgG2-M2 mAb (a.k.a. S2P6 IgG2 C131S C219S FLGG) comprised the SARS-CoV-2 specific heavy chain polypeptide of S2P6-IgG2-WT with the following mutations: the cysteine at position 219 in the upper hinge was substituted with serine (C219S; Eu numbering) and the residues at position 233-237 of IgG2 (PV-AG) in the lower hinge were substituted with the amino acids (EFLGG; Eu numbering) from the human IgG4 subclass. The amino acid sequence is shown in SEQ ID NO: 232 and is encoded by the codon-optimised DNA with the sequence shown as SEQ ID NO: 233.

The S2P6-IgG2-M2-H429F mAb (a.k.a. S2P6 IgG2 C131S C219S FLGG H429F) comprised the SARS-CoV-2 specific heavy chain polypeptide of S2P6-IgG2-M2 mAb with the further substitution of histidine with phenylalanine at position 429 in the CH3 region (H429F; Eu numbering). The amino acid sequence is shown in SEQ ID NO: 234 and is encoded by the codon-optimised DNA with the sequence shown as SEQ ID NO: 235.

The S2P6-IgG2-M3 mAb (a.k.a. S2P6 IgG2 C131S uHI FLGG) comprised the SARS-CoV-2 specific heavy chain polypeptide of S2P6-IgG2-WT with the following mutations: the residues ERKCCVE in the upper hinge were substituted with those of the IgG1 subclass EPKSCDKTHT (FIG. 65) and additionally the residues at position 233-237 in the lower hinge were substituted with those of an IgG4 (PV-AG to EFLGG; Eu numbering). The amino acid sequence is shown in SEQ ID NO: 236 and is encoded by the codon-optimised DNA with the sequence shown as SEQ ID NO: 237.

The S2P6-IgG2-M3-H429F mAb (a.k.a. S2P6 IgG2 C131S uH1 FLGG H429F) comprised the SARS-CoV-2 specific heavy chain polypeptide of S2P6-IgG2-M3 mAb with the further substitution of histidine with phenylalanine at position 429 in the CH3 region (H429F; Eu numbering). The amino acid sequence is shown in SEQ ID NO: 238 and is encoded by the codon-optimised DNA with the sequence shown as SEQ ID NO: 239.

### CD20 mAb Rituximab

### Rituximab Light Chain

The light chain polypeptide of rituximab comprised the CD20-specific VL domain of the anti-CD20 rituximab-IgG1-WT mAb fused to human kappa constant domain (RIT-IgG1-WT a.k.a Rituximab-WT; Example 3). The RIT-IgG1-WT light chain polypeptide comprised of the amino acid sequence as shown in SEQ ID NO: 30 and is encoded by the codon-optimised DNA with the sequence shown as SEQ ID NO: 31.

### Rituximab Heavy Chain.

The RIT-IgG1-WT (a.k.a Rituximab-WT Example 3) heavy chain polypeptide comprised, in the order of the N-terminus to the C-terminus, the CD20-specific VH domain of rituximab fused to the CH1-hinge-CH2-CH3 domains of human IgG1, as shown in SEQ ID NO: 28, and is encoded by the codon-optimised DNA with the sequence shown as SEQ ID NO: 29.

The RIT-IgG1-H429F (a.k.a known as Rituximab-H429F Example 3) heavy chain polypeptide comprised, in the order of the N-terminus to the C-terminus, the CD20-specific VH domain of rituximab fused to the CH1-hinge-CH2-CH3 domains of human IgG1, with a substitution of histidine with phenylalanine at position 429 (H429F; Eu numbering) in the CH3 region (SEQ ID NO: 222), and is encoded by the codon-optimised DNA with the sequence shown as SEQ ID NO: 223.

Production of mAbs

The mAbs light chains, or the wild-type heavy chains or variant heavy chains were produced by the synthesis of synthetic cDNA sequences using standard molecular biology methods known to those skilled in the art. All sequences were verified by DNA sequence analysis.

Expression and Purification of mAbs

All mAbs were expressed in Expi293F cells (Thermo Fisher) as described elsewhere in this application. The mAbs were purified from culture supernatant by protein A affinity chromatography (as described herein above) and further purified and characterised by size exclusion chromatography (SEC) in PBS as described herein above using a Superose 6 Increase 10/300 column. Fractions (0.5 mL) were collected and the fractions containing the monomeric H2L2 IgG peak were collected and pooled for subsequent use.

SDS-PAGE Analysis of mAbs

The purified mAbs were further characterised by SDS-PAGE by the method of Laemmli either without disulphide bond reduction or following reduction in dithiothreitol (DTT).

Antigen-Binding Analysis of mAbs.

Purified antibodies were tested by flow cytometry for antigen-binding prior to functional analysis, as previously described herein.

### Evaluation of mAb Killing Potency

Complement-dependent lysis killing of cells was measured by flow cytometry as described herein above using as the Zombie Green fixable viability kit following opsonisation of lymphoma cells with mAb.

### Results

Biochemical Characterisation of mAbs.

MAbs were purified from cell culture supernatant by protein A chromatography. Subsequent SEC analysis and purification showed that all mAbs exhibited substantially equivalent characteristics.

The rituximab-IgG1-WT and rituximab-IgG1-H429F mAbs in SEC analysis, eluted as a single peak of monomeric IgG with similar migration times (FIG. 16A). SDS-PAGE analyses (FIG. 17A) confirmed that these mAbs comprised a typical IgG configuration of two heavy and two light chains (i.e. H2L2 format) as both mAbs migrated at an apparent molecular weight of 150 kDa under non-reducing conditions and resolved to 55 kDa heavy chain and 25 kDa light chain species after reduction in DTT indicating the presence of the expected inter heavy chain disulphide bonds but importantly also confirming covalent bonding of the light chain to the heavy chains in all mAbs.

All S2P6 mAbs showed equivalent characteristics in SEC and SDS-PAGE analysis (FIG. 86, FIG. 87, FIG. 88 and FIG. 89). In SEC purification all mAbs eluted as a single peak with similar SEC migration times irrespective of the IgG heavy chain subclass or whether the mAb comprised wild-type or mutated heavy chains (FIG. 86, FIG. 88).

SDS-PAGE analysis revealed that the S2P6 mAbs based on IgG1 heavy chains (i.e. mAbs S2P6-IgG1-WT and S2P6-IgG1-H429F) migrated at an expected apparent molecular weight of approximately 150 kDa confirming H2L2 configuration which after dithiothreitol (DTT) reduction resolved to the expected 55 kDa H chain and 25 kDa light chain species (FIG. 87, FIG. 89).

Similarly, mAbs S2P6-IgG2-WT and S2P6-IgG2-H429F with heavy chains based on the IgG2 subclass showed the expected H2L2 configuration with apparent molecular sizes of approximately 150 kDa (FIG. 89). However, and importantly, the IgG2-based mAbs having unconstrained hinges with various mutations in the CH1 domain and upper hinge (i.e. S2P6-IgG2-M1, S2P6-IgG2-M1-H429F S2P6-IgG2-M2, S2P6-IgG2-M2-H429F, S2P6-IgG2-M3, and S2P6-IgG2-M3-H429F) all showed H2L2 configuration with apparent molecular sizes of approximately 150 kDa (FIG. 89) that resolved after disulfide bond reduction with DTT to the 55 kDa heavy chain and 25 kDa light chain species. This indicates that these mAbs form disulfide bonds between heavy chains (H-H) and between heavy and light chains (H-L).

Interestingly also, is that prior to reduction with DTT, the S2P6-IgG2-WT and S2P6-IgG2-H429F, in which all three cysteines of the CH1 and the upper hinge are present for disulfide bonding, thus constraining the upper hinges, and that differ only by the point mutation H429F, exhibit slightly smaller apparent molecular sizes than that of the other IgG2-based mutants all of which have unconstrained upper hinges (FIG. 89A).

Evaluation of Binding of Rituximab-Based mAbs to Ramos and Ramos-S Cells

All mAbs were tested for direct antigen binding to Ramos-S cells. The CD20 mAbs RIT-IgG1-WT and RIT-IgG1-H429F demonstrated equivalent binding to Ramos cells (FIG. 14) and also to Ramos-S cells which express the SARS-CoV-2 Spike protein. EC50 values for binding were as follows: RIT-IgG1-WT EC50=2 μg/ml and RIT-IgG1-H429F EC50=1.5 μg/ml (FIG. 90A).

Evaluation of the Cell Killing of Rituximab-Based mAbs

The RIT-IgG1-WT mediates effective CDC of Ramos cells (FIG. 19E) and as seen in FIG. 90B it also mediates similarly potent CDC of Ramos-S cells. The inclusion of the H429F mutation in the rituximab IgG1 heavy chain in mAb RIT-IgG1-H429F enhanced the CDC killing of Ramos cells (FIG. 19E) and also of Ramos-S cells (FIG. 90B). Thus, Ramos cells and Ramos-S cells exhibit similar sensitivity to antibody mediated CDC.

Evaluation of Binding of S2P6 mAbs to Ramos-S Cells

All S2P6 mAbs showed equivalent binding to the Ramos-S cells. The EC50 values of binding were similar ranging from 0.10 μg/mL for S2P6-IgG1-H429F to 0.15 μg/mL (S2P6-IgG2-WT) (FIG. 91).

### Evaluation of the Cell Killing Potency

The S2P6-IgG1-WT failed to exhibit detectable CDC (FIGS. 92A and 92D). However, the incorporation of the H429F mutation into the IgG1 heavy chain in mAb S2P6-G1-H429F resulted in a substantial but limited improvement in CDC killing, showing an EC50=0.40 μg/mL and AUC of 67.7. However, this improvement in CDC potency was limited as only 49% of cells were killed at 10 μg/mL (FIG. 92A), as also observed in FIG. 49D.

The S2P6-IgG2-WT not surprisingly failed to show any CDC activity (FIG. 92A, 92D) which is consistent with the known lack of effector potency of IgG2 subclass heavy chains. The incorporation of the H429F mutation into the IgG2 backbone in the S2P6-IgG2-H429F mAb resulted in only a very small improvement in CDC, with only 9% of cells killed at 10 μg/mL and an AUC of 11.5, which contrasted with the greater killing by its IgG1-based equivalent (S2P6-IgG1-H429F) which killed approximately half the Ramos-S cells at this concentration (FIG. 92A).

The distinct effects of the hexamerising point mutation, H429F, on markedly improving the killing potency of S2P6 mAbs formatted with the heavy chain of the IgG1 subclass (S2P6-IgG1-H429F) but poorly in S2P6 mAbs formatted with the heavy chain of the IgG2 subclass (S2P6-IgG2-H429F) indicates that in addition to the H429F mutation, other features may be required to confer more potent killing activity on mAbs that are based on the IgG2 subclass. It was next assessed whether combining other mutations with H429F can effectively increase killing activity on mAbs that are based on the IgG2 subclass. Accordingly, modifications to the CH1 domain and to the upper and lower hinge of IgG2 were incorporated into IgG2-based mAbs along with the H429F mutation. The mAbs S2P6-IgG2-M1-H429F, S2P6-IgG2-M2-H429F, S2P6-IgG2-M3-H429F (Table 13) were evaluated for killing potency on Ramos-S cells (FIG. 92). All three mAbs showed greatly enhanced CDC compared to S2P6-IgG2-H429F. Thus, mAbs S2P6-IgG2-M1-H429F mediated a kill at 10 μg/mL of 85%, (EC50=0.64 μg/mL and AUC of 139.6), S2P6-IgG2-M2-H429F showed a kill at 10 μg/mL of 87% (EC50=0.17 μg/mL and AUC of 139), and S2P6-IgG2-M3-H429F showed kill at 10 ug/ml of 89% (EC50=0.23 μg/mL and AUC of 135.7) compared to the poor killing achieved by S2P6-IgG2-H429F that lacked the CH1 and upper hinge mutations and showed kill at 10 μg/mL of only 9% (AUC of only 11.5) (FIGS. 92B and 92D).

It was also remarkable that the CH1/hinge mutations on the IgG2 backbone yielded S2P6 mAbs with greater potency than the S2P6 based on the IgG1 backbone (i.e S2P6-IgG1-H429F mAb) (FIG. 92A, 92B and 92D). Thus, for example, analysis of CDC by mAb S2P6-IgG2-M1-H429F showed an EC50=0.64 μg/mL and killing of 85% of cells at 10 μg/mL with an AUC of 139.6 compared to S2P6-IgG1-H429F which showed a lower potency with an EC50=0.4 μg/mL and lower cell killing of 49% at 10 μg/mL, as well as a lower AUC=67.7. The S2P6-IgG2-M2-H429F and S2P6-IgG2-M3-H429F mAbs showed a similar magnitude of improvement as S2P6-IgG2-M1-H429F when compared to S2P6-IgG1-H429F (FIG. 92C, 92D).

To define the contributions of the CH1 and hinge mutations as well as the H429F mutation to the surprising enhancement of killing, a further set of mAbs was examined. These mAbs comprised IgG2 backbones that incorporated the M1, M2, and M3 mutations (FIG. 65 and Table 13) but lacked the H429F mutation (i.e. mAbs S2P6-IgG2-M1, S2P6-IgG2-M2, S2P6-IgG2-M3) (Table 13).

All three mAbs failed to exhibit appreciable killing of the Ramos-S cells (FIG. 92C, 92D) with maximum kills at 10 μg/mL for S2P6-IgG2-M1, S2P6-IgG2-M2, S2P6-IgG2-M3 of 11%, 6%, and 3% respectively, and AUC values of 9.3, 2.6, and 1.1, respectively (FIG. 92C, 92D).

Thus, taking into account the % kill at 10 μg/mL, EC50 and AUC values of each mAb the order of decreasing potency is S2P6-IgG2-M1-H429F ~S2P6-IgG2-M2-H429F ~S2P6-IgG2-M3-H429F >>S2P6-IgG1-HF >>>S2P6-IgG2-M1 ~S2P6-IgG2-M2,~S2P6-IgG2-M3,~IgG2-H429F >S2P6-IgG2-WT.

### Discussion

The most sequence diverse areas of IgG1 and IgG2, and IgG subclasses generally, are within the hinge. The hinge region is believed to provide structural flexibility between the Fab and Fc portions of the immunoglobulin molecule, but they are also the site of structural constraints. The core hinge of all IgG subclasses contains cysteine residues that form inter heavy chain covalent disulfide bonds. The lower hinge is important in interactions of IgG3, IgG1 and IgG4 with effector molecules of the immune system such as Fc gamma receptors and C1q. However, the upper hinge which is the most sequence-diverse part of the hinge (Table 7, 8 and 9) provides different structural constraints. In IgG1 the upper hinge is the only site of disulphide bonding of the light chain to the heavy chain which occurs at cysteine 220 of the heavy chain. (FIG. 3, FIG. 65)

IgG2 is unique and structurally distinct. Unlike IgG1, it has three cysteine residues that provide multiple sites of heavy and light chain disulphide bonding and additional inter-heavy chain disulphide bond formation outside of the core hinge. Two cysteines occur in the upper hinge, Cys219 and Cys220 (the equivalent of Cys220 in IgG1), and a third is located in the CH1 domain at position 131 (Cys131; note in IgG1 this reside is Ser131). All three cysteines in an IgG2 heavy chain can be utilised in disulphide bonding. In a typical H2L2 configured IgG2, the six cysteine residues comprising three in each heavy chain, form disulphide bonds between heavy and light chains. Cys219 and Cys220 can form inter chain bonds between upper hinges of the two heavy chains and Cys131 of CH1 and Cys219 in the upper hinge can form an unusual intra-heavy chain bond (Dillon et al., 2008, supra). Thus, preparations of wildtype human IgG2 antibodies contain mixtures of structural isoforms (named IgG2-A, IgG2-B, and IgG2-A/B) that arise because of the symmetrical or asymmetrical arrangement of these disulfide bonds in individual IgG2 molecules.

It is clear that for mAbs with an IgG2 backbone, very potent cytotoxic activity can be achieved by a combination of an unconstrained upper hinge with a permissive lower hinge and the incorporation of the hexamerising mutation, H429F. Indeed, such mAbs achieved cytotoxic effects even greater than IgG1 heavy chains containing hexamerising mutations.

This is particularly interesting since IgG1 is the archetypal subclass for optimal effector functions such as FcγR activation, C1q binding, and activation of the classical complement pathway. It already comprises an unconstrained upper hinge and a permissive lower hinge (ELLGG), and indeed the incorporation of hexamerising mutations confers substantial improvement in CDC. In this example, IgG2 mutated to remove the constraining disulphide bonds within the upper hinge without the incorporation of a hexamerising mutation failed to mediate substantial cytotoxicity. Conversely, IgG2 heavy chain containing only a hexamerising mutation also failed to mediate substantial cytotoxicity. However, the combination of mutations to remove the constraining disulphide bonds within the upper hinge paired with the incorporation of a hexamerising mutation, H429F, resulted in a remarkable transformation killing potency.

TABLE 13
List of mAbs based on S2P6 used or contemplated in this study

| | | Heavy | | | | | | |
|---|---|---|---|---|---|---|---|---|
| | | Chain | Heavy chain Region (amino acid | | | | | |
| | Heavy Chain | Codon- | numbering based on Eu numbering) | | | | | |
| | Amino Acid | Optimized | | Upper hinge | | Lower hinge | | |
| mAb | SEQ ID | DNA SEQ | CH1 | (UH) | | (LH) | CH3 | CH3 |
| Name | NO: | ID NO: | 131 | 219 | 220 | 233-237 | 429 | 430 |
| S2P6- | SEQ ID NO: | SEQ ID | S | S | C | ELLGG | H | E |
| IgG1-WT | 78 | NO: 79 | | | | | | |
| S2P6- | SEQ ID | SEQ ID | S | S | C | ELLGG | F | E |
| IgG1- | NO: 90 | NO: 91 | | | | | | |
| H429F | | | | | | | | |
| S2P6- | SEQ ID NO: | SEQ ID | C | C | C | PV-AG | H | E |
| IgG2-WT | 224 | NO: 225 | | | | | | |
| S2P6- | SEQ ID NO: | SEQ ID | C | C | C | PV-AG | F | E |
| IgG2- | 226 | NO: 227 | | | | | | |
| H429F | | | | | | | | |
| S2P6- | SEQ ID NO: | SEQ ID | S | C | S | EFLGG | H | E |
| IgG2-M1 | 228 | NO: 229 | | | | | | |
| S2P6- | SEQ ID NO: | SEQ ID | S | C | S | EFLGG | F | E |
| IgG2-M1- | 230 | NO: 231 | | | | | | |
| H429F | | | | | | | | |
| S2P6- | SEQ ID NO: | SEQ ID | S | S | C | EFLGG | H | E |
| IgG2-M2 | 232 | NO: 233 | | | | | | |
| S2P6- | SEQ ID NO: | SEQ ID | S | S | C | EFLGG | F | E |
| IgG2-M2- | 234 | NO: 235 | | | | | | |
| H429F | | | | | | | | |
| S2P6- | SEQ ID NO: | SEQ ID | S | S* | C* | EFLGG | H | E |
| IgG2-M3 | 236 | NO: 237 | | | | | | |
| S2P6- | SEQ ID NO: | SEQ ID | S | S* | C* | EFLGG | F | E |
| IgG2-M3- | 238 | NO: 239 | | | | | | |
| H429F | | | | | | | | |

*in the mAbs S2P6-IgG2-M3, S2P6-IgG2-M3-H429F, residues S219 and C220 are contained within the upper hinge of IgGI that is present in these mAbs.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

It will be appreciated by those skilled in the art that the immunotherapeutic proteins, uses and pharmaceutical composition disclosed herein are not restricted by the particular application(s) described. Neither are the proteins, uses and pharmaceutical composition restricted in their preferred embodiment(s) with regard to the particular elements and/or features described or depicted herein. It will also be appreciated that the proteins, uses and pharmaceutical composition disclosed herein are not limited to the embodiment or embodiments disclosed, but are capable of numerous rearrangements, modifications and substitutions without departing from the scope of the disclosure as set forth and defined by the following claims.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

This application claims priority from Australian Provisional Application No. 2023901425 entitled “Immunotherapeutic proteins” filed on 10 May 2023, the entire contents of which are hereby incorporated by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### Example 24 Grouping of IgG and Mutant Variants by CDC in Comparison to IgG2-WT

Rituximab (Rit), Isatuximab (Isa) and S2P6 IgG2-WT and mutants were categorised according to lytic activity by comparison to that of IgG2-WT. Categories are shown in FIG. 93. Group 1 contains those mAbs with highest CDC activity (EC50 values below or equal to 0.5 μg/ml and levels of target killing above 50%), Group 2 for intermediate CDC activity, and Group 3 for CDC activity still above that of IgG2-WT but less than Group 1 or Group 2. MAbs with activity equivalent to IgG2-WT are denoted as ‘equivalent’ (eq) and if less than IgG2-WT as Group 4.

Grouping of IgG2 mAb mutants by CDC activity. The binding of subcomponent C1q of the C1 complex (FIGS. 71, 78, 85), comprising C1q and the proteases (C1r2C1s2), to a target opsonised by IgG, or an IgG immune complex, is the initiating stage of activation of the complement system. Activation of complement by C1 results in the deposition of complement fragments C4b and C3b, and subsequent further proteolyzed fragments of these, and finally results in the assembly of the membrane attack complex and the lysis and death of susceptible targets. Antibodies comprising mutated heavy chains derived from the human IgG2 subclass were evaluated for their capacity to kill Ramos or Raji cells in the presence of normal human serum (FIGS. 64, 70, 76, 77, 83, 84, 92). Based on these activities mAbs were grouped by comparison to the CDC activity of human IgG2-WT as a baseline. IgG2-WT is widely known not to effectively lyse opsonised mammalian cell targets, especially in comparison to the IgG1 and IgG3 subclasses.

Group 4: CDC activity less than the CDC activity mediated by human IgG2. Although human IgG2 mediates very low levels of measurable CDC, less than 20% target killing mediated by 10 μg/ml herein, some examined mutants mediated comparable or even lower levels of killing. Surprisingly the mAb comprising the isatuximab variable domains and a HC composed of the V_{H} joined to a human IgG2 constant region with the double mutation Q274K and H429F (Isa-IgG2-Q274K-H429F) mediated less (FIG. 83C) or comparable (FIG. 84C) levels of CDC compared to IgG2-WT.

Group 3: CDC activity is moderately enhanced over that mediated by human IgG2. Group 3 IgG2 variants with only moderately enhanced CDC over that of the IgG2-WT mAb format were Rit-IgG2-H429F, Isa-IgG2-H429F, S2P6-IgG2-H429F, Isa-IgG2-EFLGG, Rit-IgG2-EFLGG-H429F, Rit-IgG2-ELLGG-H429F, Isa-IgG2-M3 and Isa-IgG2-LALA-H429F.

Group 2: CDC is substantially enhanced over the CDC activity mediated by human IgG2. Group 2 IgG2 isatuximab variants with only substantially enhanced CDC over that of the IgG2-WT mAb format were Isa-IgG2-H429F, Isa-IgG2-EFLGG-H429F, Isa-IgG2-M1, and Isa-IgG2-M2.

Group 1: CDC is greatly enhanced over the CDC activity mediated by human IgG2. Group 1 IgG2 variants mediated the highest level of CDC with EC50 values below or equal to 0.5 μg/ml and levels of target killing above 50% in the model systems used herein. CDC Group 1 comprised the CD38 specific mAbs Isa-IgG2-M1-H429F, Isa-IgG2-M2-H429F and Isa-IgG2-M3-H429F, and the SARS-COV-2 specific mAbs S2P6-IgG2-M1-H429F, S2P6-IgG2-M2-H429F and S2P6-IgG2-M3-H429F. This group of highly efficient CDC mediating mAbs have the common properties of an IgG2 backbone with modification of the upper and lower hinge regions, into which the additional H429F hexamerising mutation has been introduced. It is also notable that the lysis of target cells has been mediated by the recognition of disparate targets, CD38 and a viral spike protein, suggesting that these formats are highly CDC active independent of target antigen.

TABLE 14
SEQUENCES

SEQ ID NO: 3
IgG1 H chain CH1-hinge-CH2-CH3 (Protein)
>J00228 IgG1
ASTKGPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQPENNYKTTPPVLDSDGSFFLYSK
LTVDKSRWQQGNVFSCSVMHEALHNHYTQKSLSLSPGK
SEQ ID NO: 4
IgG3 H chain (NB hinge 4 repeat form) (Protein)
>X03604-IGHG3
ASTKGPSVFPLAPCSRSTSGGTAALGCLVKDYFPEPVTVSWNSGALTSGVHTFPAVLQSSGLYSLSSVVTVPS
SSLGTQTYTCNVNHKPSNTKVDKRVELKTPLGDTTHTCPRCPEPKSCDTPPPCPRCPEPKSCDTPPPCPRCPE
PKSCDTPPPCPRCPAPELLGGPSVFLFPPKPKDTLMISRTPEVTCVVVDVSHEDPEVQFKWYVDGVEVHNAKT
KPREEQYNSTFRVVSVLTVLHQDWLNGKEYKCKVSNKALPAPIEKTISKTKGQPREPQVYTLPPSREEMTKNQ
VSLTCLVKGFYPSDIAVEWESSGQPENNYNTTPPMLDSDGSFFLYSKLTVDKSRWQQGNIFSCSVMHEALHNR
FTQKSLSLSPGK
SEQ ID NO: 5
IgG4 H chain (Protein)
>K01316-IGHG4
ASTKGPSVFPLAPCSRSTSESTAALGCLVKDYFPEPVTVSWNSGALTSGVHTFPAVLQSSGLYSLSSVVTVPS
SSLGTKTYTCNVDHKPSNTKVDKRVESKYGPPCPSCPAPEFLGGPSVFLFPPKPKDTLMISRTPEVTCVVVDV
SQEDPEVQFNWYVDGVEVHNAKTKPREEQFNSTYRVVSVLTVLHQDWLNGKEYKCKVSNKGLPSSIEKTISKA
KGQPREPQVYTLPPSQEEMTKNQVSLTCLVKGFYPSDIAVEWESNGQPENNYKTTPPVLDSDGSFFLYSRLTV
DKSRWQEGNVFSCSVMHEALHNHYTQKSLSLSLGK
SEQ ID NO: 6
IgG2 H chain (Protein)
>J00230-IGHG2
ASTKGPSVFPLAPCSRSTSESTAALGCLVKDYFPEPVTVSWNSGALTSGVHTFPAVLQSSGLYSLSSVVTVPS
SNFGTQTYTCNVDHKPSNTKVDKTVERKCCVECPPCPAPPVAGPSVFLFPPKPKDTLMISRTPEVTCVVVDVS
HEDPEVQFNWYVDGVEVHNAKTKPREEQFNSTFRVVSVLTVVHQDWLNGKEYKCKVSNKGLPAPIEKTISKTK
GQPREPQVYTLPPSREEMTKNQVSLTCLVKGFYPSDIAVEWESNGQPENNYKTTPPMLDSDGSFFLYSKLTVD
KSRWQQGNVFSCSVMHEALHNHYTQKSLSLSPGK
SEQ ID NO: 7
IgA1 H chain (Protein)
>J00220 IGHA1
ASPTSPKVFPLSLCSTQPDGNVVIACLVQGFFPQEPLSVTWSESGQGVTARNFPPSQDASGDLYTTSSQLTLP
ATQCLAGKSVTCHVKHYTNPSQDVTVPCPVPSTPPTPSPSTPPTPSPSCCHPRLSLHRPALEDLLLGSEANLT
CTLTGLRDASGVTFTWTPSSGKSAVQGPPERDLCGCYSVSSVLPGCAEPWNHGKTFTCTAAYPESKTPLTATL
SKSGNTFRPEVHLLPPPSEELALNELVTLTCLARGESPKDVLVRWLQGSQELPREKYLTWASRQEPSQGTTTF
AVTSILRVAAEDWKKGDTFSCMVGHEALPLAFTQKTIDRLAGKPTHVNVSVVMAEVDGTCY
SEQ ID NO: 8
IgA2 H chain (Protein)
>J00221 IGHA2
ASPTSPKVFPLSLDSTPQDGNVVVACLVQGFFPQEPLSVTWSESGQNVTARNFPPSQDASGDLYTTSSQLTLP
ATQCPDGKSVTCHVKHYTNPSQDVTVPCPVPPPPPCCHPRLSLHRPALEDLLLGSEANLTCTLTGLRDASGAT
FTWTPSSGKSAVQGPPERDLCGCYSVSSVLPGCAQPWNHGETFTCTAAHPELKTPLTANITKSGNTFRPEVHL
LPPPSEELALNELVTLTCLARGESPKDVLVRWLQGSQELPREKYLTWASRQEPSQGTTTFAVTSILRVAAEDW
KKGDTFSCMVGHEALPLAFTQKTIDRLAGKPTHVNVSVVMAEVDGTCY
SEQ ID NO: 9
IgE CH4 (Protein)
>IGHE CH4 J00222
GPRAAPEVYAFATPEWPGSRDKRTLACLIQNFMPEDISVQWLHNEVQLPDARHSTTQPRKTKGSGFFVFSRLE
VTRAEWEQKDEFICRAVHEAASPSQTVQRAVSVNPGK
SEQ ID NO: 10
IgM CH4 (Protein)
>IGHM CH4 X57331
GVALHRPDVYLLPPAREQLNLRESATITCLVTGFSPADVFVQWMQRGQPLSPEKYVTSAPMPEPQAPGRYFAH
SILTVSEEEWNTGETYTCVVAHEALPNRVTERTVDKSTGKPTLYNVSLVMSDTAGTCY
SEQ ID NO: 11
IgD CH3 (Protein)
>IGHD K02879
AAQAPVKLSLNLLASSDPPEAASWLLCEVSGFSPPNILLMWLEDQREVNTSGFAPARPPPQPRSTTFWAWSVL
RVPAPPSPQPATYTCVVSHEDSRTLLNASRSLEVSYVTDHGPMK
SEQ ID NO: 12
Trastuzumab heavy chain (Protein)
EVQLVESGGGLVQPGGSLRLSCAASGFNIKDTYIHWVRQAPGKGLEWVARIYPTNGYTRYADSVKGRFTISAD
TSKNTAYLQMNSLRAEDTAVYYCSRWGGDGFYAMDYWGQGTLVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVKENWYVDGVEVHNAKT
KPREEQYNSTYRVVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVFSCSVMHEALHNH
YTQKSLSLSPGK*
SEQ ID NO: 13
Trastuzumab heavy chain DNA
GAAGTGCAGCTGGTTGAATCTGGCGGCGGACTGGTTCAACCAGGCGGATCTCTGAGACTGAGCTGTGCCGCCA
GCGGCTTCAACATCAAGGACACCTACATCCACTGGGTCCGACAGGCCCCTGGCAAAGGACTTGAATGGGTCGC
CAGAATCTACCCCACCAACGGCTACACCAGATACGCCGACTCTGTGAAGGGCAGATTCACCATCAGCGCCGAC
ACCAGCAAGAACACCGCCTACCTGCAGATGAACAGCCTGAGAGCCGAGGACACCGCCGTGTACTACTGTAGTA
GATGGGGAGGCGACGGCTTCTACGCTATGGATTATTGGGGCCAGGGCACCCTGGTCACAGTTAGCTCTGCGTC
GACAAAAGGCCCTAGCGTCTTTCCACTCGCCCCATCCTCCAAGAGCACCTCTGGGGGCACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAG
CTTGGGTACCCAGACCTATATCTGCAATGTCAATCATAAACCCAGTAACACAAAGGTAGACAAGAAAGTCGAA
CCCAAATCTTGCGACAAAACTCACACATGCCCTCCTTGCCCCGCCCCCGAACTCCTGGGGGGACCTTCGGTTT
TTCTTTTTCCACCTAAACCGAAAGACACCCTGATGATCAGCAGGACACCAGAAGTGACATGTGTCGTCGTGGA
CGTATCCCATGAGGACCCGGAGGTGAAGTTCAACTGGTATGTTGATGGTGTGGAAGTGCATAATGCAAAAACT
AAGCCACGGGAGGAACAGTACAATAGCACATACAGAGTAGTTAGCGTTCTGACTGTCCTGCACCAAGATTGGC
TGAACGGGAAGGAATACAAGTGTAAGGTGAGCAACAAAGCCCTGCCCGCTCCAATTGAGAAAACAATTTCTAA
GGCCAAAGGACAGCCCCGGGAGCCACAGGTGTATACTCTGCCGCCTAGCAGGGAGGAAATGACCAAGAACCAG
GTGAGCCTGACCTGTCTGGTGAAAGGCTTCTACCCCAGCGATATCGCAGTTGAGTGGGAGAGCAATGGCCAAC
CCGAGAACAACTACAAGACTACGCCCCCCGTGCTTGATAGCGACGGATCATTTTTCCTGTACTCAAAACTGAC
CGTGGACAAAAGCAGATGGCAGCAGGGAAACGTTTTCAGTTGCTCAGTGATGCACGAAGCGCTGCACAATCAT
TATACTCAGAAAAGCCTGAGCTTGAGCCCGGGAAAATGA
SEQ ID NO: 14
Trastuzumablightchain(Protein)
DIQMTQSPSSLSASVGDRVTITCRASQDVNTAVAWYQQKPGKAPKLLIYSASFLYSGVPSRFSGSRSGTDFTL
TISSLQPEDFATYYCQQHYTTPPTFGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSENRGEC*
SEQ ID NO: 15
Trastuzumab light chain DNA
GATATCCAGATGACACAGAGCCCCAGCAGCCTGTCTGCCTCTGTGGGAGACAGAGTGACCATCACCTGTAGAG
CCAGCCAGGACGTGAACACAGCCGTGGCTTGGTATCAGCAGAAGCCTGGCAAGGCCCCTAAGCTGCTGATCTA
CAGCGCCAGCTTTCTGTACAGCGGCGTGCCCAGCAGATTCAGCGGAAGCAGAAGCGGCACCGACTTCACCCTG
ACCATAAGCAGTCTGCAGCCCGAGGACTTCGCCACCTACTACTGTCAGCAGCACTACACCACACCTCCAACCT
TCGGCCAGGGTACCAAGGTGGAAATCAAGCGGACAGTGGCCGCTCCTAGCGTGTTCATCTTTCCACCTAGCGA
CGAGCAGCTGAAGTCTGGCACAGCCTCTGTCGTGTGCCTGCTGAACAACTTCTACCCCAGAGAAGCCAAGGTG
CAGTGGAAGGTGGACAACGCCCTGCAGAGCGGCAATAGCCAAGAGAGCGTGACCGAGCAGGACAGCAAGGACT
CTACCTACAGCCTGAGCAGCACCCTGACACTGAGCAAGGCCGACTACGAGAAGCACAAAGTGTACGCCTGCGA
AGTGACCCACCAGGGCCTTTCTAGCCCTGTGACCAAGAGCTTCAACCGGGGCGAGTGCTAA
SEQ ID NO: 16
11B8 heavy Chain (Protein)
EVQLVESGGGLVQPGRSLRLSCAASGFTENDYAMHWVRQAPGKGLEWVSTISWNSGSIGYADSVKGRFTISRD
NAKKSLYLQMNSLRAEDTALYYCAKDIQYGNYYYGMDVWGQGTLVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKVDKK
VEPKSCDKTHTCPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVFSCSVMHEALH
NHYTQKSLSLSPGK*
SEQ ID NO: 17
11B8 heavy Chain DNA
GAAGTGCAGCTGGTTGAATCTGGCGGCGGACTGGTTCAGCCTGGCAGATCTCTGAGACTGAGCTGTGCCGCCA
GCGGCTTCACCTTCAACGATTACGCCATGCACTGGGTCCGACAGGCCCCTGGAAAAGGCCTTGAATGGGTGTC
CACCATCAGCTGGAACAGCGGCTCTATCGGCTACGCCGATTCCGTGAAGGGCAGATTCACCATCTCCAGAGAC
AACGCCAAGAAGTCCCTGTACCTGCAGATGAACAGCCTGAGAGCCGAGGACACAGCCCTGTACTACTGCGCCA
AGGACATCCAGTACGGCAACTACTACTACGGCATGGACGTGTGGGGCCAGGGCACACTGGTCACAGTTAGCTC
TGCTAGCACAAAAGGCCCTAGCGTCTTTCCACTCGCCCCATCCTCCAAGAGCACCTCTGGGGGCACTGCGGCT
TTGGGCTGCTTGGTGAAGGATTACTTCCCAGAGCCTGTCACTGTCAGCTGGAATAGCGGGGCTCTGACCAGTG
GAGTGCACACCTTTCCCGCCGTGTTACAGAGCAGCGGCCTCTATAGCCTGAGCAGTGTGGTAACTGTGCCCTC
GAGCAGCTTGGGTACCCAGACCTATATCTGCAATGTCAATCATAAACCCAGTAACACAAAGGTAGACAAGAAA
GTCGAACCCAAATCTTGCGACAAAACTCACACATGCCCTCCTTGCCCCGCCCCCGAACTCCTGGGGGGACCTT
CGGTTTTTCTTTTTCCACCTAAACCGAAAGACACCCTGATGATCAGCAGGACACCAGAAGTGACATGTGTCGT
CGTGGACGTATCCCATGAGGACCCGGAGGTGAAGTTCAACTGGTATGTTGATGGTGTGGAAGTGCATAATGCA
AAAACTAAGCCACGGGAGGAACAGTACAATAGCACATACAGAGTAGTTAGCGTTCTGACTGTCCTGCACCAAG
ATTGGCTGAACGGGAAGGAATACAAGTGTAAGGTGAGCAACAAAGCCCTGCCCGCTCCAATTGAGAAAACAAT
TTCTAAGGCCAAAGGACAGCCCCGGGAGCCACAGGTGTATACTCTGCCGCCTAGCAGGGAGGAAATGACCAAG
AACCAGGTGAGCCTGACCTGTCTGGTGAAAGGCTTCTACCCCAGCGATATCGCAGTTGAGTGGGAGAGCAATG
GCCAACCCGAGAACAACTACAAGACTACGCCCCCCGTGCTTGATAGCGACGGATCATTTTTCCTGTACTCAAA
ACTGACCGTGGACAAAAGCAGATGGCAGCAGGGAAACGTTTTCAGTTGCTCAGTGATGCACGAAGCGCTGCAC
AATCATTATACTCAGAAAAGCCTGAGCTTGAGCCCGGGAAAATGA
SEQ ID NO: 18
11B8 Light Chain (Protein)
EIVLTQSPATLSLSPGERATLSCRASQSVSSYLAWYQQKPGQAPRLLIYDASNRATGIPARFSGSGSGTDFTL
TISSLEPEDFAVYYCQQRSNWPITFGQGTRLEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSENRGEC*
SEQ ID NO: 19
11B8 Light Chain DNA
GAAATTGTGCTGACACAGAGCCCCGCCACACTGTCACTTTCTCCAGGCGAAAGAGCCACACTGAGCTGCAGAG
CCAGCCAGAGCGTGTCCTCTTACCTGGCCTGGTATCAGCAGAAGCCTGGACAGGCTCCCCGGCTGCTGATCTA
CGATGCCAGCAATAGAGCCACCGGCATTCCCGCCAGATTTTCTGGCTCTGGAAGCGGCACCGACTTCACCCTG
ACCATAAGCAGCCTGGAACCTGAGGACTTCGCCGTGTACTACTGCCAGCAGAGAAGCAACTGGCCCATCACCT
TTGGCCAGGGTACCAGACTGGAAATCAAGCGGACAGTGGCCGCTCCTAGCGTGTTCATCTTTCCACCTAGCGA
CGAGCAGCTGAAGTCTGGCACAGCCTCTGTCGTGTGCCTGCTGAACAACTTCTACCCCAGAGAAGCCAAGGTG
CAGTGGAAGGTGGACAACGCCCTGCAGAGCGGCAATAGCCAAGAGAGCGTGACCGAGCAGGACAGCAAGGACT
CTACCTACAGCCTGAGCAGCACCCTGACACTGAGCAAGGCCGACTACGAGAAGCACAAAGTGTACGCCTGCGA
AGTGACCCACCAGGGCCTTTCTAGCCCTGTGACCAAGAGCTTCAACCGGGGCGAGTGCTAA
SEQ ID NO: 20
Daratumumab Heavy Chain (Protein)
EVQLLESGGGLVQPGGSLRLSCAVSGFTFNSFAMSWVRQAPGKGLEWVSAISGSGGGTYYADSVKGRFTISRD
NSKNTLYLQMNSLRAEDTAVYFCAKDKILWFGEPVFDYWGQGTLVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKVDKR
VEPKSCDKTHTCPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQGNVFSCSVMHEALH
NHYTQKSLSLSPGK*
SEQ ID NO: 21
Daratumumab Heavy Chain DNA
GAAGTTCAGCTGCTGGAATCTGGCGGCGGACTGGTTCAACCAGGCGGATCTCTGAGACTGAGCTGTGCCGTGT
CCGGCTTCACCTTCAACAGCTTCGCCATGAGCTGGGTCCGACAGGCCCCTGGAAAAGGCCTTGAATGGGTGTC
CGCCATCTCTGGCTCTGGCGGAGGCACATATTACGCCGACTCTGTGAAGGGCAGATTCACCATCAGCCGGGAC
AACAGCAAGAACACCCTGTACCTGCAGATGAACAGCCTGAGAGCCGAGGATACCGCCGTGTACTTCTGCGCCA
AGGACAAGATCCTTTGGTTCGGCGAGCCCGTGTTCGATTATTGGGGCCAGGGCACCCTGGTCACAGTTAGCTC
TGCTAGCACAAAAGGCCCTAGCGTCTTTCCACTCGCCCCATCCTCCAAGAGCACCTCTGGGGGCACTGCGGCT
TTGGGCTGCTTGGTGAAGGATTACTTCCCAGAGCCTGTCACTGTCAGCTGGAATAGCGGGGCTCTGACCAGTG
GAGTGCACACCTTTCCCGCCGTGTTACAGAGCAGCGGCCTCTATAGCCTGAGCAGTGTGGTAACTGTGCCCTC
GAGCAGCTTGGGTACCCAGACCTATATCTGCAATGTCAATCATAAACCCAGTAACACAAAGGTAGACAAGAGA
GTCGAACCCAAATCTTGCGACAAAACTCACACATGCCCTCCTTGCCCCGCCCCCGAACTCCTGGGGGGACCTT
CGGTTTTTCTTTTTCCACCTAAACCGAAAGACACCCTGATGATCAGCAGGACACCAGAAGTGACATGTGTCGT
CGTGGACGTATCCCATGAGGACCCGGAGGTGAAGTTCAACTGGTATGTTGATGGTGTGGAAGTGCATAATGCA
AAAACTAAGCCACGGGAGGAACAGTACAATAGCACATACAGAGTAGTTAGCGTTCTGACTGTCCTGCACCAAG
ATTGGCTGAACGGGAAGGAATACAAGTGTAAGGTGAGCAACAAAGCCCTGCCCGCTCCAATTGAGAAAACAAT
TTCTAAGGCCAAAGGACAGCCCCGGGAGCCACAGGTGTATACTCTGCCGCCTAGCAGGGAGGAAATGACCAAG
AACCAGGTGAGCCTGACCTGTCTGGTGAAAGGCTTCTACCCCAGCGATATCGCAGTTGAGTGGGAGAGCAATG
GCCAACCCGAGAACAACTACAAGACTACGCCCCCCGTGCTTGATAGCGACGGATCATTTTTCCTGTACTCAAA
ACTGACCGTGGACAAAAGCAGATGGCAGCAGGGAAACGTTTTCAGTTGCTCAGTGATGCACGAAGCGCTGCAC
AATCATTATACTCAGAAAAGCCTGAGCTTGAGCCCGGGAAAATGA
SEQ ID NO: 22
Daratumumab Light Chain (Protein)
EIVLTQSPATLSLSPGERATLSCRASQSVSSYLAWYQQKPGQAPRLLIYDASNRATGIPARFSGSGSGTDFTL
TISSLEPEDFAVYYCQQRSNWPPTFGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSENRGEC*
SEQ ID NO: 23
Daratumumab Light Chain DNA
GAAATTGTGCTGACACAGAGCCCCGCCACACTGTCACTTTCTCCAGGCGAAAGAGCCACACTGAGCTGCAGAG
CCAGCCAGAGCGTGTCCTCTTACCTGGCCTGGTATCAGCAGAAGCCTGGACAGGCTCCCCGGCTGCTGATCTA
CGATGCCAGCAATAGAGCCACCGGCATTCCCGCCAGATTTTCTGGCTCTGGAAGCGGCACCGACTTCACCCTG
ACCATAAGCAGCCTGGAACCTGAGGACTTCGCCGTGTACTACTGCCAGCAGAGAAGCAACTGGCCTCCTACCT
TTGGCCAGGGTACCAAGGTGGAAATCAAGCGGACAGTGGCCGCTCCTAGCGTGTTCATCTTTCCACCTAGCGA
CGAGCAGCTGAAGTCTGGCACAGCCTCTGTCGTGTGCCTGCTGAACAACTTCTACCCCAGAGAAGCCAAGGTG
CAGTGGAAGGTGGACAACGCCCTGCAGAGCGGCAATAGCCAAGAGAGCGTGACCGAGCAGGACAGCAAGGACT
CTACCTACAGCCTGAGCAGCACCCTGACACTGAGCAAGGCCGACTACGAGAAGCACAAAGTGTACGCCTGCGA
AGTGACCCACCAGGGCCTTTCTAGCCCTGTGACCAAGAGCTTCAACCGGGGCGAGTGCTAA
SEQ ID NO: 24
Pertuzumab heavy chain (Protein)
EVQLVESGGGLVQPGGSLRLSCAASGFTFTDYTMDWVRQAPGKGLEWVADVNPNSGGSIYNQRFKGRFTLSVD
RSKNTLYLQMNSLRAEDTAVYYCARNLGPSFYFDYWGQGTLVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKENWYVDGVEVHNAKTK
PREEQYNSTYRVVSVLTVLHQDWLNGKEYKCKVSNKALPAPIEKTISKAKGQPREPQVYTLPPSREEMTKNQV
SLTCLVKGFYPSDIAVEWESNGQPENNYKTTPPVLDSDGSFFLYSKLTVDKSRWQQGNVFSCSVMHEALHNHY
TQKSLSLSPGK*
SEQ ID NO: 25
Pertuzumab heavy chain DNA
GAAGTGCAGCTGGTTGAATCTGGCGGCGGACTGGTTCAACCAGGCGGATCTCTGAGACTGAGCTGTGCCGCCA
GCGGCTTCACCTTCACCGACTACACAATGGACTGGGTCCGACAGGCCCCTGGCAAAGGACTTGAATGGGTCGC
CGACGTGAACCCCAATAGCGGCGGCAGCATCTACAACCAGCGGTTCAAGGGCAGATTCACCCTGAGCGTGGAC
AGAAGCAAGAACACCCTGTACCTGCAGATGAACAGCCTGAGAGCCGAGGACACCGCCGTGTACTACTGCGCCA
GAAATCTGGGCCCCAGCTTCTACTTCGATTATTGGGGCCAGGGCACCCTGGTCACAGTTAGCTCTGCTAGCAC
AAAAGGCCCTAGCGTCTTTCCACTCGCCCCATCCTCCAAGAGCACCTCTGGGGGCACTGCGGCTTTGGGCTGC
TTGGTGAAGGATTACTTCCCAGAGCCTGTCACTGTCAGCTGGAATAGCGGGGCTCTGACCAGTGGAGTGCACA
CCTTTCCCGCCGTGTTACAGAGCAGCGGCCTCTATAGCCTGAGCAGTGTGGTAACTGTGCCCTCGAGCAGCTT
GGGTACCCAGACCTATATCTGCAATGTCAATCATAAACCCAGTAACACAAAGGTAGACAAGAAAGTCGAACCC
AAATCTTGCGACAAAACTCACACATGCCCTCCTTGCCCCGCCCCCGAACTCCTGGGGGGACCTTCGGTTTTTC
TTTTTCCACCTAAACCGAAAGACACCCTGATGATCAGCAGGACACCAGAAGTGACATGTGTCGTCGTGGACGT
ATCCCATGAGGACCCGGAGGTGAAGTTCAACTGGTATGTTGATGGTGTGGAAGTGCATAATGCAAAAACTAAG
CCACGGGAGGAACAGTACAATAGCACATACAGAGTAGTTAGCGTTCTGACTGTCCTGCACCAAGATTGGCTGA
ACGGGAAGGAATACAAGTGTAAGGTGAGCAACAAAGCCCTGCCCGCTCCAATTGAGAAAACAATTTCTAAGGC
CAAAGGACAGCCCCGGGAGCCACAGGTGTATACTCTGCCGCCTAGCAGGGAGGAAATGACCAAGAACCAGGTG
AGCCTGACCTGTCTGGTGAAAGGCTTCTACCCCAGCGATATCGCAGTTGAGTGGGAGAGCAATGGCCAACCCG
AGAACAACTACAAGACTACGCCCCCCGTGCTTGATAGCGACGGATCATTTTTCCTGTACTCAAAACTGACCGT
GGACAAAAGCAGATGGCAGCAGGGAAACGTTTTCAGTTGCTCAGTGATGCACGAAGCGCTGCACAATCATTAT
ACTCAGAAAAGCCTGAGCTTGAGCCCGGGAAAATGA
SEQ ID NO: 26
Pertuzumab light chain (Protein)
DIQMTQSPSSLSASVGDRVTITCKASQDVSIGVAWYQQKPGKAPKLLIYSASYRYTGVPSRFSGSGSGTDFTL
TISSLQPEDFATYYCQQYYIYPYTFGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSENRGEC*
SEQ ID NO: 27
Pertuzumab light chain DNA
GATATCCAGA5TGACACAGAGCCCCAGCAGCCTGTCTGCCTCTGTGGGAGACAGAGTGACCATCACCTGTAAA
GCCAGCCAGGACGTGTCCATCGGCGTGGCATGGTATCAGCAGAAGCCTGGCAAGGCCCCTAAGCTGCTGATCT
ACAGCGCCAGCTACAGATACACCGGCGTGCCCAGCAGATTTTCTGGCAGCGGCTCTGGCACCGACTTCACCCT
GACCATATCTAGCCTGCAGCCTGAGGACTTCGCCACCTACTACTGCCAGCAGTACTACATCTACCCCTACACC
TTCGGCCAGGGTACCAAGGTGGAAATCAAGCGGACAGTGGCCGCTCCTAGCGTGTTCATCTTTCCACCTAGCG
ACGAGCAGCTGAAGTCTGGCACAGCCTCTGTCGTGTGCCTGCTGAACAACTTCTACCCCAGAGAAGCCAAGGT
GCAGTGGAAGGTGGACAACGCCCTGCAGAGCGGCAATAGCCAAGAGAGCGTGACCGAGCAGGACAGCAAGGAC
TCTACCTACAGCCTGAGCAGCACCCTGACACTGAGCAAGGCCGACTACGAGAAGCACAAAGTGTACGCCTGCG
AAGTGACCCACCAGGGCCTTTCTAGCCCTGTGACCAAGAGCTTCAACCGGGGCGAGTGCTAA
SEQ ID NO: 28
Rituximab Heavy Chain (Protein)
QVQLQQPGAELVKPGASVKMSCKASGYTFTSYNMHWVKQTPGRGLEWIGAIYPGNGDTSYNQKFKGKATLTAD
KSSSTAYMQLSSLTSEDSAVYYCARSTYYGGDWYFNVWGAGTTVTVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGNVFSCSVMHEALHN
HYTQKSLSLSPGK*
SEQ ID NO: 29
Rituximab Heavy Chain DNA
CAGGTTCAGCTGCAACAGCCTGGCGCCGAGCTTGTGAAACCTGGCGCCTCTGTGAAGATGAGCTGCAAGGCCA
GCGGCTACACCTTCACCAGCTACAACATGCACTGGGTCAAGCAGACCCCTGGCAGAGGCCTGGAATGGATCGG
AGCCATCTATCCCGGCAACGGCGACACCTCCTACAACCAGAAGTTCAAGGGCAAAGCCACACTGACCGCCGAC
AAGAGCAGCAGCACAGCCTACATGCAGCTGAGCAGCCTGACCAGCGAAGATAGCGCCGTGTACTACTGCGCCA
GAAGCACCTATTACGGCGGCGACTGGTACTTCAACGTGTGGGGAGCTGGCACCACCGTGACAGTTTCTGCCGC
TAGCACAAAAGGCCCTAGCGTCTTTCCACTCGCCCCATCCTCCAAGAGCACCTCTGGGGGCACTGCGGCTTTG
GGCTGCTTGGTGAAGGATTACTTCCCAGAGCCTGTCACTGTCAGCTGGAATAGCGGGGCTCTGACCAGTGGAG
TGCACACCTTTCCCGCCGTGTTACAGAGCAGCGGCCTCTATAGCCTGAGCAGTGTGGTAACTGTGCCCTCGAG
CAGCTTGGGTACCCAGACCTATATCTGCAATGTCAATCATAAACCCAGTAACACAAAGGTAGACAAGAAAGTC
GAACCCAAATCTTGCGACAAAACTCACACATGCCCTCCTTGCCCCGCCCCCGAACTCCTGGGGGGACCTTCGG
TTTTTCTTTTTCCACCTAAACCGAAAGACACCCTGATGATCAGCAGGACACCAGAAGTGACATGTGTCGTCGT
GGACGTATCCCATGAGGACCCGGAGGTGAAGTTCAACTGGTATGTTGATGGTGTGGAAGTGCATAATGCAAAA
ACTAAGCCACGGGAGGAACAGTACAATAGCACATACAGAGTAGTTAGCGTTCTGACTGTCCTGCACCAAGATT
GGCTGAACGGGAAGGAATACAAGTGTAAGGTGAGCAACAAAGCCCTGCCCGCTCCAATTGAGAAAACAATTTC
TAAGGCCAAAGGACAGCCCCGGGAGCCACAGGTGTATACTCTGCCGCCTAGCAGGGATGAACTGACCAAGAAC
CAGGTGAGCCTGACCTGTCTGGTGAAAGGCTTCTACCCCAGCGATATCGCAGTTGAGTGGGAGAGCAATGGCC
AACCCGAGAACAACTACAAGACTACGCCCCCCGTGCTTGATAGCGACGGATCATTTTTCCTGTACTCAAAACT
GACCGTGGACAAAAGCAGATGGCAGCAGGGAAACGTTTTCAGTTGCTCAGTGATGCACGAAGCGCTGCACAAT
CATTATACTCAGAAAAGCCTGAGCTTGAGCCCGGGAAAATGA
SEQ ID NO: 30
Rituximab Light Chain (Protein)
QIVLSQSPAILSASPGEKVTMTCRASSSVSYIHWFQQKPGSSPKPWIYATSNLASGVPVRESGSGSGTSYSLT
ISRVEAEDAATYYCQQWTSNPPTFGGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSSPVTKSENRGEC*
SEQ ID NO: 31
Rituximab Light Chain DNA
CAAATTGTGCTGTCTCAGAGCCCCGCCATCCTGAGTGCTTCTCCAGGCGAGAAAGTGACCATGACCTGCAGAG
CCAGCAGCAGCGTGTCCTACATCCACTGGTTCCAGCAGAAGCCTGGCAGCAGCCCTAAGCCTTGGATCTACGC
CACAAGCAATCTGGCCAGCGGCGTGCCAGTCAGATTTTCTGGCTCTGGCAGCGGCACCAGCTACAGCCTGACA
ATCAGCAGAGTGGAAGCCGAGGATGCCGCCACCTACTACTGTCAGCAGTGGACCAGCAATCCTCCTACCTTTG
GCGGCGGTACCAAGGTGGAAATCAAGCGGACAGTGGCCGCTCCTAGCGTGTTCATCTTTCCACCTAGCGACGA
GCAGCTGAAGTCTGGCACAGCCTCTGTCGTGTGCCTGCTGAACAACTTCTACCCCAGAGAAGCCAAGGTGCAG
TGGAAGGTGGACAACGCCCTGCAGAGCGGCAATAGCCAAGAGAGCGTGACCGAGCAGGACAGCAAGGACTCTA
CCTACAGCCTGAGCAGCACCCTGACACTGAGCAAGGCCGACTACGAGAAGCACAAAGTGTACGCCTGCGAAGT
GACCCACCAGGGCCTTTCTAGCCCTGTGACCAAGAGCTTCAACCGGGGCGAGTGCTAA
SEQ ID NO: 32
A TNP IgG1 heavy chain (protein)
EVQLQESGPSLVKPSQTLSLTCSVTGDSITSGYWNWIRQFPGNKLEYMGFINYSGNTYYNPSLRSRISITRDT
SKNQYFLHLNSVTTEDTATYYCARANWDVFAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMHEALHNHYTQK
SLSLSPGK*
SEQ ID NO: 33
A TNP-IgG1 heavy chain DNA
GAGGTGCAGCTTCAGGAGTCAGGACCTAGCCTCGTGAAACCTTCTCAGACTCTGTCCCTCACATGTTCTGTCA
CTGGCGACTCCATCACCAGTGGTTACTGGAACTGGATCCGGCAATTCCCAGGGAATAAACTTGAGTACATGGG
TTTCATAAATTACAGTGGTAACACTTACTACAATCCATCTCTCAGAAGTCGAATCTCCATCACTCGAGACACA
TCCAAGAACCAGTACTTCCTGCACTTGAATTCTGTGACTACTGAGGACACAGCCACATATTACTGTGCAAGGG
CTAACTGGGACGTCTTTGCTTACTGGGGCCAAGGGACTCTGGTCACTGTCTCTGCAGCCTCCACCAAGGGCCC
ATCGGTCTTCCCCCTGGCACCCTCCTCCAAGAGCACCTCTGGGGGCACAGCGGCCCTGGGCTGCCTGGTCAAG
GACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCA
GACCTACATCTGCAACGTGAATCACAAGCCCAGCAACACCAAGGTGGACAAGAAAGTTGAGCCCAAATCTTGT
GACAAAACTCACACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGGCTGAATGGCAAGG
AGTACAAGTGCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTGACC
TGCCTGGTCAAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACCGTGGACAAGAG
CAGGTGGCAGCAGGGGAACGTCTTCTCATGCTCCGTGATGCATGAGGCTCTGCACAACCACTACACGCAGAAG
AGCCTCTCCCTGTCTCCGGGTAAATGA
SEQ ID NO: 34
A TNP IgG2 heavy chain (protein)
EVQLQESGPSLVKPSQTLSLTCSVTGDSITSGYWNWIRQVPGNKLEYMGFINYSGNTYYNPSLRSRISITRDT
SKNQYFLHLNSVTTEDTATYYCARANWDVFAYWGQGTLVTVSAASTKGPSVFPLAPCSRSTSESTAALGCLVK
DYFPEPVTVSWNSGALTSGVHTFPAVLQSSGLYSLSSVVTVPSSNFGTQTYTCNVDHKPSNTKVDKTVERKCC
VECPPCPAPPVAGPSVFLFPPKPKDTLMISRTPEVTCVVVDVSHEDPEVQFNWYVDGVEVHNAKTKPREEQEN
STFRVVSVLTVVHQDWLNGKEYKCKVSNKGLPAPIEKTISKTKGQPREPQVYTLPPSREEMTKNQVSLTCLVK
GFYPSDIAVEWESNGQPENNYKTTPPMLDSDGSFFLYSKLTVDKSRWQQGNVFSCSVMHEALHNHYTQKSLSL
SLGK
SEQ ID NO: 35
A TNP IgG2 heavy chain (DNA)
GAGGTGCAGTTGCAGGAATCTGGTCCCAGTCTAGTTAAGCCCAGCCAGACACTGAGCCTGACATGTAGTGTGA
CCGGTGACAGCATTACAAGCGGCTACTGGAACTGGATCAGACAGGTGCCAGGAAACAAACTCGAATACATGGG
GTTCATCAATTACAGTGGCAATACTTACTATAATCCCAGCCTGAGAAGCAGAATTTCTATAACCAGAGACACC
AGCAAAAACCAGTACTTTCTGCACCTGAACAGCGTAACAACGGAAGACACCGCCACCTATTACTGCGCCAGGG
CCAACTGGGATGTGTTCGCATACTGGGGCCAGGGCACTCTGGTGACGGTGAGCGCCGCGTCGACAAAAGGCCC
TAGCGTCTTTCCACTCGCCCCATGTTCAAGAAGCACCAGCGAGTCAACTGCGGCTTTGGGCTGCTTGGTGAAG
GATTACTTCCCAGAGCCTGTCACTGTCAGCTGGAATAGCGGGGCTCTGACCAGTGGAGTGCACACCTTTCCCG
CCGTGTTACAGAGCAGCGGCCTCTATAGCCTGAGCAGTGTGGTAACTGTGCCCTCGAGCAATTTTGGTACCCA
GACCTATACATGCAATGTCGATCATAAACCCAGTAACACAAAGGTAGACAAGACAGTCGAAAGAAAATGCTGT
GTGGAGTGCCCTCCTTGCCCCGCCCCCCCAGTGGCCGGGCCTTCGGTTTTTCTTTTTCCACCTAAACCGAAAG
ACACCCTGATGATCAGCAGGACACCAGAAGTGACATGTGTCGTCGTGGACGTATCCCATGAAGACCCGGAGGT
GCAATTCAACTGGTATGTTGATGGTGTGGAAGTGCATAATGCAAAAACTAAGCCACGGGAGGAACAGTTCAAT
AGCACATTCAGAGTAGTTAGCGTTCTGACTGTCGTGCACCAAGATTGGCTGAACGGGAAGGAATACAAGTGTA
AGGTGAGCAACAAAGGTCTGCCCGCTCCAATTGAGAAAACAATTTCTAAGACCAAAGGACAGCCCCGGGAGCC
ACAGGTGTATACTCTGCCGCCTAGCAGGGAGGAAATGACCAAGAACCAGGTGAGCCTGACCTGTCTGGTGAAA
GGCTTCTACCCCAGCGATATCGCAGTTGAGTGGGAGAGCAATGGCCAACCCGAGAACAACTACAAGACTACGC
CCCCCATGCTTGATAGCGACGGATCATTTTTCCTGTACTCAAAACTGACCGTGGACAAAAGCAGATGGCAGCA
GGGAAACGTTTTCAGTTGCTCAGTGATGCACGAAGCgCTGCACAATCATTATACTCAGAAAAGCCTGAGCTTG
AGCTTAGGAAAATGA
SEQ ID NO: 36
A TNP-light chain (protein)
DIVMSQSPSSLAVSVGEKVTMSCKSSQSLLYSSNQKNYLAWYQRKPGQSPKLLIYWASTRESGVPDRFTGSGS
GTDFTLTISSVKAEDLAVYYCQHYYSSPYTFGGGTKLEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSENRGE
C
SEQ ID NO: 37
A TNP-light chain (DNA)
GACATAGTGATGTCACAGAGCCCAAGCAGCCTTGCAGTGTCTGTTGGTGAGAAGGTGACCATGAGCTGCAAGT
CAAGCCAGAGCCTGCTCTATAGCAGCAATCAGAAGAACTATTTGGCCTGGTATCAGAGAAAACCCGGCCAGAG
CCCTAAGCTCCTTATTTATTGGGCTAGCACACGCGAGTCCGGCGTGCCGGACAGATTTACCGGCAGCGGTAGC
GGCACCGATTTTACTTTGACCATCTCGTCTGTAAAGGCCGAAGACCTGGCAGTGTATTACTGTCAACATTATT
ACTCATCTCCCTACACTTTCGGAGGGGGGACCAAACTGGAAATTAAGCGTACGGTTGCTGCCCCTTCTGTCTT
CATCTTCCCTCCCAGCGATGAACAGCTGAAAAGTGGGACAGCGAGTGTAGTGTGCCTGCTAAACAATTTTTAC
CCACGGGAGGCCAAAGTGCAGTGGAAAGTGGACAACGCTCTGCAAAGTGGAAATTCTCAGGAGTCCGTCACAG
AGCAGGACTCGAAGGACAGCACTTACAGCTTGTCAAGCACCCTGACGCTGAGCAAAGCCGATTACGAGAAGCA
CAAGGTGTACGCATGCGAAGTTACGCACCAAGGACTGAGTAGCCCAGTCACAAAGAGCTTCAACAGGGGGGAA
TGT
SEQ ID NO: 38
Rituximab IgG3 heavy chain (protein)
QVQLQQPGAELVKPGASVKMSCKASGYTFTSYNMHWVKQTPGRGLEWIGAIYPGNGDTSYNQKFKGKATLTAD
KSSSTAYMQLSSLTSEDSAVYYCARSTYYGGDWYFNVWGAGTTVTVSAASTKGPSVFPLAPCSRSTSGGTAAL
GCLVKDYFPEPVTVSWNSGALTSGVHTFPAVLQSSGLYSLSSVVTVPSSSLGTQTYTCNVNHKPSNTKVDKRV
ELKTPLGDTTHTCPRCPEPKSCDTPPPCPRCPEPKSCDTPPPCPRCPEPKSCDTPPPCPRCPAPELLGGPSVF
LFPPKPKDTLMISRTPEVTCVVVDVSHEDPEVQFKWYVDGVEVHNAKTKLREEQYNSTFRVVSVLTVLHQDWL
NGKEYKCKVSNKALPAPIEKTISKTKGQPREPQVYTLPPSREEMTKNQVSLTCLVKGFYPSDIAVEWESNGQP
ENNYKTTPPVLDSDGSFFLYSKLTVDKSRWQQGNIFSCSVMHEALHNHYTQKSLSLSPGK
SEQ ID NO: 39
Rituximab IgG3 heavy chain (DNA)
CAGGTTCAGCTGCAACAGCCTGGCGCCGAGCTTGTGAAACCTGGCGCCTCTGTGAAGATGAGCTGCAAGGCCA
GCGGCTACACCTTCACCAGCTACAACATGCACTGGGTCAAGCAGACCCCTGGCAGAGGCCTGGAATGGATCGG
AGCCATCTATCCCGGCAACGGCGACACCTCCTACAACCAGAAGTTCAAGGGCAAAGCCACACTGACCGCCGAC
AAGAGCAGCAGCACAGCCTACATGCAGCTGAGCAGCCTGACCAGCGAAGATAGCGCCGTGTACTACTGCGCCA
GAAGCACCTATTACGGCGGCGACTGGTACTTCAACGTGTGGGGAGCTGGTACCACCGTGACAGTTTCTGCCGC
TAGCACAAAAGGCCCTAGCGTCTTTCCACTCGCCCCATGTTCTAGAAGCACCTCTGGGGGCACTGCGGCTTTG
GGCTGCTTGGTGAAGGATTACTTCCCAGAGCCTGTCACTGTCAGCTGGAATAGCGGGGCTCTGACCAGTGGAG
TGCACACCTTTCCCGCCGTGTTACAGAGCAGCGGCCTCTATAGCCTGAGCAGTGTGGTAACTGTGCCCTCGAG
CAGCTTGGGCACCCAGACCTATACCTGCAATGTCAATCATAAACCCAGTAACACAAAGGTAGACAAGCGGGTC
GAACTGAAAACCCCCCTGGGCGACACCACCCACACCTGTCCTAGATGCCCCGAGCCCAAGTCCTGCGATACCC
CTCCACCTTGCCCCAGGTGCCCTGAGCCTAAGAGCTGTGATACCCCCCCTCCCTGCCCTCGGTGTCCAGAGCC
TAAGTCTTGTGACACCCCACCCCCATGTCCAAGGTGCCCCGCCCCCGAACTCCTGGGGGGACCTTCGGTTTTT
CTTTTTCCACCTAAACCGAAAGACACCCTGATGATCAGCAGGACACCAGAAGTGACATGTGTCGTCGTGGACG
TATCCCATGAGGACCCGGAGGTGCAGTTCAAGTGGTATGTTGATGGTGTGGAAGTGCATAATGCAAAAACTAA
GCTGCGGGAGGAACAGTACAATAGCACATTCAGAGTAGTTAGCGTTCTGACTGTCCTGCACCAAGATTGGCTG
AACGGGAAGGAATACAAGTGTAAGGTGAGCAACAAAGCCCTGCCCGCTCCAATTGAGAAAACAATTTCTAAGA
CCAAAGGACAGCCCCGGGAGCCACAGGTGTATACTCTGCCGCCTAGCAGGGAAGAGATGACCAAGAACCAGGT
GAGCCTGACCTGTCTGGTGAAAGGCTTCTACCCCAGCGATATCGCAGTTGAGTGGGAGAGCAATGGCCAACCC
GAGAACAACTACAAGACTACGCCCCCCGTGCTTGATAGCGACGGATCATTTTTCCTGTACTCAAAACTGACCG
TGGACAAAAGCAGATGGCAGCAGGGAAACATCTTCAGTTGCTCAGTGATGCACGAAGCGCTGCACAATCATTA
TACTCAGAAAAGCCTGAGCTTGAGCCCGGGAAAA
SEQ ID NO: 40
Rituximab IgG4 heavy chain (protein)
QVQLQQPGAELVKPGASVKMSCKASGYTFTSYNMHWVKQTPGRGLEWIGAIYPGNGDTSYNQKFKGKATLTAD
KSSSTAYMQLSSLTSEDSAVYYCARSTYYGGDWYFNVWGAGTTVTVSAASTKGPSVFPLAPCSRSTSESTAAL
GCLVKDYFPEPVTVSWNSGALTSGVHTFPAVLQSSGLYSLSSVVTVPSSSLGTKTYTCNVDHKPSNTKVDKRV
ESKYGPPCPSCPAPEFLGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQVYTLPPSQEEMTKNQVS
LTCLVKGFYPSDIAVEWESNGQPENNYKTTPPVLDSDGSFFLYSRLTVDKSRWQEGNVFSCSVMHEALHNHYT
QKSLSLSLGK
SEQ ID NO: 41
Rituximab IgG4 heavy chain (DNA)
CAGGTTCAGCTGCAACAGCCTGGCGCCGAGCTTGTGAAACCTGGCGCCTCTGTGAAGATGAGCTGCAAGGCCA
GCGGCTACACCTTCACCAGCTACAACATGCACTGGGTCAAGCAGACCCCTGGCAGAGGCCTGGAATGGATCGG
AGCCATCTATCCCGGCAACGGCGACACCTCCTACAACCAGAAGTTCAAGGGCAAAGCCACACTGACCGCCGAC
AAGAGCAGCAGCACAGCCTACATGCAGCTGAGCAGCCTGACCAGCGAAGATAGCGCCGTGTACTACTGCGCCA
GAAGCACCTATTACGGCGGCGACTGGTACTTCAACGTGTGGGGAGCTGGtACCACCGTGACAGTTTCTGCCGC
TAGCACAAAAGGCCCTAGCGTCTTTCCACTCGCCCCATgtTCCAgaAGCACCTCTGAAAGTACTGCGGCTTTG
GGCTGCTTGGTGAAGGATTACTTCCCAGAGCCTGTCACTGTCAGCTGGAATAGCGGGGCTCTGACCAGTGGAG
TGCACACCTTTCCCGCCGTGTTACAGAGCAGCGGCCTCTATAGCCTGAGCAGTGTGGTAACTGTGCCCTCGAG
CAGCTTGGGcACCaAGACCTATAcaTGCAATGTCqAcCATAAACCCAGTAACACAAAGGTAGACAAGcgAGTC
GAAAGCAAGTACGGCCCCCCCTGCCCTAGCTGCCCCGCCCCCGAAtTtCTGGGGGGACCTTCGGTTTTTCTTT
TTCCACCTAAACCGAAAGACACCCTGATGATCAGCAGGACACCAGAAGTGACATGTGTCGTCGTGGACGTATC
CCAgGAGGACCCGGAGGTGcAGTTCAACTGGTATGTTGATGGTGTGGAAGTGCATAATGCAAAAACTAAGCCA
CGGGAGGAACAGTtCAATAGCACATACAGAGTAGTTAGCGTTCTGACTGTCCTGCACCAAGATTGGCTGAACG
GGAAGGAATACAAGTGTAAGGTGAGCAACAAAGgCCTGCCCagTagcATTGAGAAAACAATTTCTAAGGCCAA
AGGACAGCCCCGGGAGCCACAGGTGTATACTCTGCCGCCTAGCcaaGAgGAAaTGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCGTGCTTGATAGCGACGGATCATTTTTCCTGTACTCAcggCTGACCGTGGA
CAAAAGCAGATGGCAGgAGGGAAACGTTTTCAGTTGCTCAGTGATGCACGAAGCGCTGCACAATCATTATACT
CAGAAAAGCCTGAGCTTGAGCttaGGAAAA
SEQ ID NO: 42
Isatuximab heavy chain (Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVKENWYVDGVEVHNAKT
KPREEQYNSTYRVVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVFSCSVMHEALHNH
YTQKSLSLSPGK
SEQ ID NO: 43
Isatuximab heavy chain DNA
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGCACCTCCGTCACAGTTAGCTCTGCTAG
CACAAAAGGCCCTAGCGTCTTTCCACTCGCCCCATCCTCCAAGAGCACCTCTGGGGGCACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAG
CTTGGGTACCCAGACCTATATCTGCAATGTCAATCATAAACCCAGTAACACAAAGGTAGACAAGAAAGTCGAA
CCCAAATCTTGCGACAAAACTCACACATGCCCTCCTTGCCCCGCCCCCGAACTCCTGGGGGGACCTTCGGTTT
TTCTTTTTCCACCTAAACCGAAAGACACCCTGATGATCAGCAGGACACCAGAAGTGACATGTGTCGTCGTGGA
CGTATCCCATGAGGACCCGGAGGTGAAGTTCAACTGGTATGTTGATGGTGTGGAAGTGCATAATGCAAAAACT
AAGCCACGGGAGGAACAGTACAATAGCACATACAGAGTAGTTAGCGTTCTGACTGTCCTGCACCAAGATTGGC
TGAACGGGAAGGAATACAAGTGTAAGGTGAGCAACAAAGCCCTGCCCGCTCCAATTGAGAAAACAATTTCTAA
GGCCAAAGGACAGCCCCGGGAGCCACAGGTGTATACTCTGCCGCCTAGCAGGGAGGAAATGACCAAGAACCAG
GTGAGCCTGACCTGTCTGGTGAAAGGCTTCTACCCCAGCGATATCGCAGTTGAGTGGGAGAGCAATGGCCAAC
CCGAGAACAACTACAAGACTACGCCCCCCGTGCTTGATAGCGACGGATCATTTTTCCTGTACTCAAAACTGAC
CGTGGACAAAAGCAGATGGCAGCAGGGAAACGTTTTCAGTTGCTCAGTGATGCACGAAGCGCTGCACAATCAT
TATACTCAGAAAAGCCTGAGCTTGAGCCCGGGAAAATGA
SEQ ID NO: 44
Isatuximab light chain (Protein)
DIVMTQSHLSMSTSLGDPVSITCKASQDVSTVVAWYQQKPGQSPRRLIYSASYRYIGVPDRFTGSGAGTDFTF
TISSVQAEDLAVYYCQQHYSPPYTFGGGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSENRGEC
SEQ ID NO: 45
Isatuximab light chain DNA
GATATCGTGATGACACAGAGCCACCTGAGCATGAGCACCAGCCTGGGAGATCCCGTGTCCATCACCTGTAAAG
CCAGCCAGGATGTGTCCACCGTGGTGGCCTGGTATCAGCAGAAGCCTGGCCAGTCTCCTCGGCGGCTGATCTA
CTCTGCCAGCTACAGATATATCGGCGTGCCCGACAGATTCACCGGATCTGGCGCTGGCACCGACTTCACCTTT
ACCATCAGCTCTGTGCAGGCCGAGGACCTGGCCGTGTACTATTGTCAGCAGCACTACAGCCCTCCATACACCT
TTGGCGGCGGTACCAAGCTGGAAATCAAGCGGACAGTGGCCGCTCCTAGCGTGTTCATCTTTCCACCTAGCGA
CGAGCAGCTGAAGTCTGGCACAGCCTCTGTCGTGTGCCTGCTGAACAACTTCTACCCCAGAGAAGCCAAGGTG
CAGTGGAAGGTGGACAACGCCCTGCAGAGCGGCAATAGCCAAGAGAGCGTGACCGAGCAGGACAGCAAGGACT
CTACCTACAGCCTGAGCAGCACCCTGACACTGAGCAAGGCCGACTACGAGAAGCACAAAGTGTACGCCTGCGA
AGTGACCCACCAGGGCCTTTCTAGCCCTGTGACCAAGAGCTTCAACCGGGGCGAGTGCTAA
SEQ ID NO: 46
BDR5-1WT heavy chain (protein) (HC-hDR5-01-G56T SEQ ID
NO: 36 of U.S. Pat. No. 10,882,913)
EVQLQQSGAEVVKPGASVKLSCKASGFNIKDTFIHWVKQAPGQGLEWIGRIDPANTNTKYDPKFQGKATITTD
TSSNTAYMELSSLRSEDTAVYYCVRGLYTYYFDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKRVEPK
SCDKTHTCPPCPAPELLGGPSVFLFPPKPKDTLMISRTPEVTCVVVDVSHEDPEVKENWYVDGVEVHNAKTKP
REEQYNSTYRVVSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMHEALHNHYT
QKSLSLSPG
SEQ ID NO: 47
BDR5-1WT heavy chain (DNA)
GAAGTGCAGCTGCAACAGTCTGGCGCCGAGGTTGTGAAACCTGGCGCCTCTGTGAAGCTGAGCTGTAAAGCCA
GCGGCTTCAACATCAAGGACACCTTCATCCACTGGGTCAAGCAGGCCCCTGGACAGGGACTCGAATGGATCGG
CAGAATCGACCCCGCCAACACCAACACTAAGTACGACCCCAAGTTCCAGGGCAAAGCCACCATCACCACCGAC
ACCAGCAGCAACACCGCCTACATGGAACTGAGCAGCCTGAGAAGCGAGGACACCGCCGTGTACTATTGTGTGC
GGGGCCTGTACACCTACTACTTCGATTATTGGGGCCAGGGTACCCTGGTCACAGTTAGCTCTGCTAGCACAAA
AGGCCCTAGCGTCTTTCCACTCGCCCCATCCTCCAAGAGCACCTCTGGGGGCACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAGCTTGGG
CACCCAGACCTATATCTGCAATGTCAATCATAAACCCAGTAACACAAAGGTAGACAAGAGAGTCGAACCCAAA
TCTTGCGACAAAACTCACACATGCCCTCCTTGCCCCGCCCCCGAACTCCTGGGGGGACCTTCGGTTTTTCTTT
TTCCACCTAAACCGAAAGACACCCTGATGATCAGCAGGACACCAGAAGTGACATGTGTCGTCGTGGACGTATC
CCATGAGGACCCGGAGGTGAAGTTCAACTGGTATGTTGATGGTGTGGAAGTGCATAATGCAAAAACTAAGCCA
CGGGAGGAACAGTACAATAGCACATACAGAGTAGTTAGCGTTCTGACTGTCCTGCACCAAGATTGGCTGAACG
GGAAGGAATACAAGTGTAAGGTGAGCAACAAAGCCCTGCCCGCTCCAATTGAGAAAACAATTTCTAAGGCCAA
AGGACAGCCCCGGGAGCCACAGGTGTATACTCTGCCGCCTAGCAGGGAGGAAATGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCGTGCTTGATAGCGACGGATCATTTTTCCTGTACTCAAAACTGACCGTGGA
CAAAAGCAGATGGCAGCAGGGAAACGTTTTCAGTTGCTCAGTGATGCACGAAGCGCTGCACAATCATTATACT
CAGAAAAGCCTGAGCTTGAGCCCGGGA
SEQ ID NO: 48
BDR5 light chain (protein) (LC-hDR5-01SEQ ID NO: 39 of
U.S. Pat. No. 10,882,913)
EIVMTQSPATLSVSPGERATLSCRASQSISNNLHWYQQKPGQAPRLLIKFASQSITGIPARFSGSGSGTEFTL
TISSLQSEDFAVYYCQQGNSWPYTFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSENRGEC
SEQ ID NO: 49
BDR5 LC light chain (DNA)
GAGATTGTGATGACACAGAGCCCCGCCACACTGTCCGTTTCTCCAGGCGAAAGAGCCACACTGAGCTGCAGAG
CCAGCCAGAGCATCAGCAACAACCTGCACTGGTATCAGCAGAAGCCCGGACAGGCTCCCAGACTGCTGATCAA
GTTTGCCAGCCAGTCCATCACAGGCATCCCCGCCAGATTTTCTGGCAGCGGCTCTGGCACCGAGTTCACCCTG
ACAATCAGCAGCCTGCAGAGCGAGGACTTCGCCGTGTACTATTGCCAGCAGGGCAACAGCTGGCCCTACACCT
TCGGCCAGGGTACCAAGCTGGAAATCAAGCGGACAGTGGCCGCTCCTAGCGTGTTCATCTTTCCACCTAGCGA
CGAGCAGCTGAAGTCTGGCACAGCCTCTGTCGTGTGCCTGCTGAACAACTTCTACCCCAGAGAAGCCAAGGTG
CAGTGGAAGGTGGACAACGCCCTGCAGAGCGGCAATAGCCAAGAGAGCGTGACCGAGCAGGACAGCAAGGACT
CTACCTACAGCCTGAGCAGCACCCTGACACTGAGCAAGGCCGACTACGAGAAGCACAAAGTGTACGCCTGCGA
AGTGACCCACCAGGGCCTTTCTAGCCCTGTGACCAAGAGCTTCAACCGGGGCGAGTGC
SEQ ID NO: 50
BDR5-2WT H chain (Protein) (VH ONLY FROM HC-hDR5-01-G56T
SEQ ID NO: 36 of U.S. Pat. No. 10,882,913)
EVQLQQSGAEVVKPGASVKLSCKASGFNIKDTFIHWVKQAPGQGLEWIGRIDPANTNTKYDPKFQGKATITTD
TSSNTAYMELSSLRSEDTAVYYCVRGLYTYYFDYWGQGTLVTVSSASTKGPSVFPLAPCSRSTSESTAALGCL
VKDYFPEPVTVSWNSGALTSGVHTFPAVLOSSGLYSLSSVVTVPSSNFGTQTYTCNVDHKPSNTKVDKTVERK
CCVECPPCPAPPVAGPSVFLFPPKPKDTLMISRTPEVTCVVVDVSHEDPEVQFNWYVDGVEVHNAKTKPREEQ
FNSTFRVVSVLTVVHQDWLNGKEYKCKVSNKGLPAPIEKTISKTKGQPREPQVYTLPPSREEMTKNQVSLTCL
VKGFYPSDIAVEWESNGQPENNYKTTPPMLDSDGSFFLYSKLTVDKSRWQQGNVFSCSVMHEALHNHYTQKSL
SLSPG
SEQ ID NO: 51 BDR5-2WT heavy chain (DNA)
GAAGTGCAGCTGCAACAGTCTGGCGCCGAGGTTGTGAAACCTGGCGCCTCTGTGAAGCTGAGCTGTAAAGCCA
GCGGCTTCAACATCAAGGACACCTTCATCCACTGGGTCAAGCAGGCCCCTGGACAGGGACTCGAATGGATCGG
CAGAATCGACCCCGCCAACACCAACACTAAGTACGACCCCAAGTTCCAGGGCAAAGCCACCATCACCACCGAC
ACCAGCAGCAACACCGCCTACATGGAACTGAGCAGCCTGAGAAGCGAGGACACCGCCGTGTACTATTGTGTGC
GGGGCCTGTACACCTACTACTTCGACTATTGGGGCCAGGGTACCCTGGTCACAGTTAGCTCTGCTAGCACAAA
AGGCCCTAGCGTCTTTCCACTCGCCCCATGTTCAAGAAGCACCAGCGAGTCAACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAATTTTGG
CACCCAGACCTATACATGCAATGTCGATCATAAACCCAGTAACACAAAGGTAGACAAGACAGTCGAAAGAAAA
TGCTGTGTGGAGTGCCCTCCTTGCCCCGCCCCCCCAGTGGCCGGGCCTTCGGTTTTTCTTTTTCCACCTAAAC
CGAAAGACACCCTGATGATCAGCAGGACACCAGAAGTGACATGTGTCGTCGTGGACGTATCCCATGAAGACCC
GGAGGTGCAATTCAACTGGTATGTTGATGGTGTGGAAGTGCATAATGCAAAAACTAAGCCACGGGAGGAACAG
TTCAATAGCACATTCAGAGTAGTTAGCGTTCTGACTGTCGTGCACCAAGATTGGCTGAACGGGAAGGAATACA
AGTGTAAGGTGAGCAACAAAGGTCTGCCCGCTCCAATTGAGAAAACAATTTCTAAGACCAAAGGACAGCCCCG
GGAGCCACAGGTGTATACTCTGCCGCCTAGCAGGGAGGAAATGACCAAGAACCAGGTGAGCCTGACCTGTCTG
GTGAAAGGCTTCTACCCCAGCGATATCGCAGTTGAGTGGGAGAGCAATGGCCAACCCGAGAACAACTACAAGA
CTACGCCCCCCATGCTTGATAGCGACGGATCATTTTTCCTGTACTCAAAACTGACCGTGGACAAAAGCAGATG
GCAGCAGGGAAACGTTTTCAGTTGCTCAGTGATGCACGAAGCGCTGCACAATCATTATACTCAGAAAAGCCTG
AGCTTGAGCCCGGGA
SEQ ID NO: 52
TDR5-1WT heavy chain (protein) (HC-hDR5-05 (SEQ ID NO: 40
of U.S. Pat. No. 10,882,913)
QVQLVQSGAEVKKPGASVKVSCKASGFNIKDTHMHWVRQAPGQRLEWIGRIDPANGNTEYDQKFQGRVTITVD
TSASTAYMELSSLRSEDTAVYYCARWGTNVYFAYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKRVEPK
SCDKTHTCPPCPAPELLGGPSVFLFPPKPKDTLMISRTPEVTCVVVDVSHEDPEVKENWYVDGVEVHNAKTKP
REEQYNSTYRVVSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMHEALHNHYT
QKSLSLSPG
SEQ ID NO: 53
TDR5-1WT heavy chain (DNA)
CAGGTTCAGCTGGTTCAGTCTGGCGCCGAAGTGAAGAAACCTGGCGCCTCTGTGAAGGTGTCCTGCAAGGCCA
GCGGCTTCAACATCAAGGACACCCACATGCACTGGGTCCGACAGGCTCCAGGACAGAGACTGGAATGGATCGG
CAGAATCGACCCCGCCAACGGCAACACCGAGTACGACCAGAAATTCCAGGGCAGAGTGACCATCACCGTGGAC
ACATCTGCCAGCACCGCCTACATGGAACTGAGCAGCCTGAGAAGCGAGGACACCGCCGTGTACTACTGTGCCA
GATGGGGCACCAACGTGTACTTCGCCTATTGGGGCCAGGGTACCCTGGTCACAGTTAGCTCTGCTAGCACAAA
AGGCCCTAGCGTCTTTCCACTCGCCCCATCCTCCAAGAGCACCTCTGGGGGCACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAGCTTGGG
CACCCAGACCTATATCTGCAATGTCAATCATAAACCCAGTAACACAAAGGTAGACAAGAGAGTCGAACCCAAA
TCTTGCGACAAAACTCACACATGCCCTCCTTGCCCCGCCCCCGAACTCCTGGGGGGACCTTCGGTTTTTCTTT
TTCCACCTAAACCGAAAGACACCCTGATGATCAGCAGGACACCAGAAGTGACATGTGTCGTCGTGGACGTATC
CCATGAGGACCCGGAGGTGAAGTTCAACTGGTATGTTGATGGTGTGGAAGTGCATAATGCAAAAACTAAGCCA
CGGGAGGAACAGTACAATAGCACATACAGAGTAGTTAGCGTTCTGACTGTCCTGCACCAAGATTGGCTGAACG
GGAAGGAATACAAGTGTAAGGTGAGCAACAAAGCCCTGCCCGCTCCAATTGAGAAAACAATTTCTAAGGCCAA
AGGACAGCCCCGGGAGCCACAGGTGTATACTCTGCCGCCTAGCAGGGAGGAAATGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCGTGCTTGATAGCGACGGATCATTTTTCCTGTACTCAAAACTGACCGTGGA
CAAAAGCAGATGGCAGCAGGGAAACGTTTTCAGTTGCTCAGTGATGCACGAAGCGCTGCACAATCATTATACT
CAGAAAAGCCTGAGCTTGAGCCCGGGA
SEQ ID NO: 54
TDR5 light chain (protein) (LC-hDR5-05 SEQ ID NO: 43 of
U.S. Pat. No. 10,882,913)
DIQLTQSPSSLSASVGDRVTITCSASSSVSYMYWYQQKPGKAPKPWIYRTSNLASGVPSRFSGSGSGTDFTLT
ISSLQPEDFATYYCQQYHSYPPTFGGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSSPVTKSENRGEC
SEQ ID NO: 55
TDR5 light chain (DNA)
GATATCCAGCTGACACAGAGCCCTAGCAGCCTGTCTGCCTCTGTGGGCGACAGAGTGACCATCACATGTAGCG
CCAGCAGCAGCGTGTCCTACATGTACTGGTATCAGCAGAAGCCCGGCAAGGCCCCTAAGCCTTGGATCTACAG
AACCAGCAATCTGGCCAGCGGCGTGCCCAGCAGATTTTCTGGTTCTGGCAGCGGCACCGACTTCACCCTGACC
ATATCTAGCCTGCAGCCTGAGGACTTCGCCACCTACTACTGCCAGCAGTACCACAGCTACCCTCCAACCTTTG
GCGGCGGTACCAAGGTGGAAATCAAGCGGACAGTGGCCGCTCCTAGCGTGTTCATCTTTCCACCTAGCGACGA
GCAGCTGAAGTCTGGCACAGCCTCTGTCGTGTGCCTGCTGAACAACTTCTACCCCAGAGAAGCCAAGGTGCAG
TGGAAGGTGGACAACGCCCTGCAGAGCGGCAATAGCCAAGAGAGCGTGACCGAGCAGGACAGCAAGGACTCTA
CCTACAGCCTGAGCAGCACCCTGACACTGAGCAAGGCCGACTACGAGAAGCACAAAGTGTACGCCTGCGAAGT
GACCCACCAGGGCCTTTCTAGCCCTGTGACCAAGAGCTTCAACCGGGGCGAGTGC
SEQ ID NO: 56
TDR5-2WT heavy chain (protein) (VH ONLY FROM HC-hDR5-05
SEQ ID NO: 40 of U.S. Pat. No. 10,882,913)
QVQLVQSGAEVKKPGASVKVSCKASGFNIKDTHMHWVRQAPGQRLEWIGRIDPANGNTEYDQKFQGRVTITVD
TSASTAYMELSSLRSEDTAVYYCARWGTNVYFAYWGQGTLVTVSSASTKGPSVFPLAPCSRSTSESTAALGCL
VKDYFPEPVTVSWNSGALTSGVHTFPAVLQSSGLYSLSSVVTVPSSNFGTQTYTCNVDHKPSNTKVDKTVERK
CCVECPPCPAPPVAGPSVFLFPPKPKDTLMISRTPEVTCVVVDVSHEDPEVQFNWYVDGVEVHNAKTKPREEQ
FNSTFRVVSVLTVVHQDWLNGKEYKCKVSNKGLPAPIEKTISKTKGQPREPQVYTLPPSREEMTKNQVSLTCL
VKGFYPSDIAVEWESNGQPENNYKTTPPMLDSDGSFFLYSKLTVDKSRWQQGNVFSCSVMHEALHNHYTQKSL
SLSPG
SEQ ID NO: 57
TDR5-2WT heavy chain (DNA)
CAGGTTCAGCTGGTTCAGTCTGGCGCCGAAGTGAAGAAACCTGGCGCCTCTGTGAAGGTGTCCTGCAAGGCCA
GCGGCTTCAACATCAAGGACACCCACATGCACTGGGTCCGACAGGCTCCAGGACAGAGACTGGAATGGATCGG
CAGAATCGACCCCGCCAACGGCAACACCGAGTACGACCAGAAATTCCAGGGCAGAGTGACCATCACCGTGGAC
ACATCTGCCAGCACCGCCTACATGGAACTGAGCAGCCTGAGAAGCGAGGACACCGCCGTGTACTACTGTGCCA
GATGGGGCACCAACGTGTACTTCGCCTATTGGGGACAGGGTACCCTCGTGACCGTTAGCTCTGCTAGCACAAA
AGGCCCTAGCGTCTTTCCACTCGCCCCATGTTCAAGAAGCACCAGCGAGTCAACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAATTTTGG
CACCCAGACCTATACATGCAATGTCGATCATAAACCCAGTAACACAAAGGTAGACAAGACAGTCGAAAGAAAA
TGCTGTGTGGAGTGCCCTCCTTGCCCCGCCCCCCCAGTGGCCGGGCCTTCGGTTTTTCTTTTTCCACCTAAAC
CGAAAGACACCCTGATGATCAGCAGGACACCAGAAGTGACATGTGTCGTCGTGGACGTATCCCATGAAGACCC
GGAGGTGCAATTCAACTGGTATGTTGATGGTGTGGAAGTGCATAATGCAAAAACTAAGCCACGGGAGGAACAG
TTCAATAGCACATTCAGAGTAGTTAGCGTTCTGACTGTCGTGCACCAAGATTGGCTGAACGGGAAGGAATACA
AGTGTAAGGTGAGCAACAAAGGTCTGCCCGCTCCAATTGAGAAAACAATTTCTAAGACCAAAGGACAGCCCCG
GGAGCCACAGGTGTATACTCTGCCGCCTAGCAGGGAGGAAATGACCAAGAACCAGGTGAGCCTGACCTGTCTG
GTGAAAGGCTTCTACCCCAGCGATATCGCAGTTGAGTGGGAGAGCAATGGCCAACCCGAGAACAACTACAAGA
CTACGCCCCCCATGCTTGATAGCGACGGATCATTTTTCCTGTACTCAAAACTGACCGTGGACAAAAGCAGATG
GCAGCAGGGAAACGTTTTCAGTTGCTCAGTGATGCACGAAGCGCTGCACAATCATTATACTCAGAAAAGCCTG
AGCTTGAGCCCGGGA
SEQ ID NO: 58
BDR5-A2WT heavy chain (protein) (VH ONLY FROM HC-hDR5-
01-G56T SEQ ID NO: 36 of U.S. Pat. No. 10,882,913)
EVQLQQSGAEVVKPGASVKLSCKASGFNIKDTFIHWVKQAPGQGLEWIGRIDPANTNTKYDPKFQGKATITTD
TSSNTAYMELSSLRSEDTAVYYCVRGLYTYYFDYWGQGTLVTVSSASPTSPKVFPLSLDSTPQDGNVVVACLV
QGFFPQEPLSVTWSESGQNVTARNFPPSQDASGDLYTTSSQLTLPATQCPDGKSVTCHVKHYTNPSQDVTVPC
PVPPPPPCCHPRLSLHRPALEDLLLGSEANLTCTLTGLRDASGATFTWTPSSGKSAVQGPPERDLCGCYSVSS
VLPGCAQPWNHGETFTCTAAHPELKTPLTANITKSGNTFRPEVHLLPPPSEELALNELVTLTCLARGESPKDV
LVRWLQGSQELPREKYLTWASRQEPSQGTTTFAVTSILRVAAEDWKKGDTFSCMVGHEALPLAFTQKTIDRLA
G
SEQ ID NO: 59
BDR5-A2WT heavy chain (DNA)
GAAGTTCAGCTGCAACAGTCTGGCGCCGAGGTTGTGAAACCTGGCGCCTCTGTGAAGCTGAGCTGTAAAGCCA
GCGGCTTCAACATCAAGGACACCTTCATCCACTGGGTCAAGCAGGCCCCTGGACAGGGACTCGAGTGGATCGG
AAGAATCGACCCCGCCAACACCAACACTAAGTACGACCCCAAGTTCCAGGGCAAAGCCACCATCACCACCGAC
ACCAGCAGCAACACCGCCTACATGGAACTGAGCAGCCTGAGAAGCGAGGACACCGCCGTGTACTATTGTGTGC
GGGGCCTGTACACCTACTACTTCGACTATTGGGGCCAGGGTACCCTCGTGACCGTTAGCTCTGCTAGCCCTAC
AAGCCCCAAGGTGTTCCCTCTGAGCCTGGATAGCACACCCCAGGACGGAAATGTGGTGGTGGCTTGTCTGGTG
CAGGGATTCTTCCCACAAGAGCCCCTGTCCGTGACTTGGAGCGAGAGCGGACAGAATGTGACCGCCAGAAACT
TCCCACCTAGCCAGGATGCCAGCGGCGATCTGTACACAACAAGCAGCCAGCTGACCCTGCCTGCCACACAGTG
TCCTGATGGCAAGAGCGTGACCTGCCACGTGAAGCACTACACAAACCCCAGCCAGGACGTGACCGTGCCTTGT
CCTGTTCCTCCTCCACCTCCATGCTGTCACCCTAGACTGAGCCTGCACAGACCCGCTCTGGAAGATCTGCTGC
TGGGCTCTGAGGCCAACCTGACCTGTACACTGACCGGCCTGAGAGATGCCTCTGGCGCCACCTTTACATGGAC
ACCTAGCAGCGGAAAGTCCGCCGTTCAGGGACCTCCTGAGAGGGATCTGTGTGGCTGTTACAGCGTGTCCTCT
GTGCTGCCTGGATGTGCCCAGCCTTGGAATCACGGCGAGACATTCACCTGTACCGCCGCTCATCCCGAGCTGA
AAACACCCCTGACCGCCAACATCACCAAGAGCGGCAATACCTTCAGACCCGAAGTGCATCTGCTGCCTCCACC
ATCTGAAGAACTGGCCCTGAACGAGCTGGTGACCCTGACATGTCTGGCCAGGGGCTTCAGCCCTAAGGATGTG
CTCGTTAGATGGCTGCAGGGCAGCCAAGAGCTGCCCAGAGAGAAGTATCTGACCTGGGCCAGTCGACAGGAAC
CTTCTCAGGGCACCACAACCTTTGCCGTGACCAGCATTCTGAGAGTGGCCGCCGAGGATTGGAAGAAGGGCGA
CACCTTTAGCTGCATGGTCGGACACGAAGCGCTGCCCCTGGCTTTCACCCAGAAAACCATCGATAGACTGGCC
GGC
SEQ ID NO: 60
TDR5-A2WT heavy chain (protein) (VH ONLY FROM HC-hDR5-05
SEQ ID NO: 40 of U.S. Pat. No. 10,882,913)
QVQLVQSGAEVKKPGASVKVSCKASGFNIKDTHMHWVRQAPGQRLEWIGRIDPANGNTEYDQKFQGRVTITVD
TSASTAYMELSSLRSEDTAVYYCARWGTNVYFAYWGQGTLVTVSSASPTSPKVFPLSLDSTPQDGNVVVACLV
QGFFPQEPLSVTWSESGQNVTARNFPPSQDASGDLYTTSSQLTLPATQCPDGKSVTCHVKHYTNPSQDVTVPC
PVPPPPPCCHPRLSLHRPALEDLLLGSEANLTCTLTGLRDASGATFTWTPSSGKSAVQGPPERDLCGCYSVSS
VLPGCAQPWNHGETFTCTAAHPELKTPLTANITKSGNTFRPEVHLLPPPSEELALNELVTLTCLARGESPKDV
LVRWLQGSQELPREKYLTWASRQEPSQGTTTFAVTSILRVAAEDWKKGDTFSCMVGHEALPLAFTQKTIDRLA
G
SEQ ID NO: 61
TDR5-A2WT heavy chain (DNA)
CAGGTTCAGCTGGTTCAGTCTGGCGCCGAAGTGAAGAAACCTGGCGCCTCTGTGAAGGTGTCCTGCAAGGCCA
GCGGCTTCAACATCAAGGACACCCACATGCACTGGGTCCGACAGGCTCCAGGACAGAGACTGGAATGGATCGG
CAGAATCGACCCCGCCAACGGCAACACCGAGTACGACCAGAAATTCCAGGGCAGAGTGACCATCACCGTGGAC
ACATCTGCCAGCACCGCCTACATGGAACTGAGCAGCCTGAGAAGCGAGGACACCGCCGTGTACTACTGTGCCA
GATGGGGCACCAACGTGTACTTCGCCTATTGGGGACAGGGTACCCTCGTGACCGTTAGCTCTGCTAGCCCTAC
AAGCCCCAAGGTGTTCCCTCTGAGCCTGGATAGCACACCCCAGGACGGAAATGTGGTGGTGGCTTGTCTGGTG
CAGGGATTCTTCCCACAAGAGCCCCTGTCCGTGACTTGGAGCGAGAGCGGACAGAATGTGACCGCCAGAAACT
TCCCACCTAGCCAGGATGCCAGCGGCGATCTGTACACAACAAGCAGCCAGCTGACCCTGCCTGCCACACAGTG
TCCTGATGGCAAGAGCGTGACCTGCCACGTGAAGCACTACACAAACCCCAGCCAGGACGTGACCGTGCCTTGT
CCTGTTCCTCCTCCACCTCCATGCTGTCACCCTAGACTGAGCCTGCACAGACCCGCTCTGGAAGATCTGCTGC
TGGGCTCTGAGGCCAACCTGACCTGTACACTGACCGGCCTGAGAGATGCCTCTGGCGCCACCTTTACATGGAC
ACCTAGCAGCGGAAAGTCCGCCGTTCAGGGACCTCCTGAGAGGGATCTGTGTGGCTGTTACAGCGTGTCCTCT
GTGCTGCCTGGATGTGCCCAGCCTTGGAATCACGGCGAGACATTCACCTGTACCGCCGCTCATCCCGAGCTGA
AAACACCCCTGACCGCCAACATCACCAAGAGCGGCAATACCTTCAGACCCGAAGTGCATCTGCTGCCTCCACC
ATCTGAAGAACTGGCCCTGAACGAGCTGGTGACCCTGACATGTCTGGCCAGGGGCTTCAGCCCTAAGGATGTG
CTCGTTAGATGGCTGCAGGGCAGCCAAGAGCTGCCCAGAGAGAAGTATCTGACCTGGGCCAGTCGACAGGAAC
CTTCTCAGGGCACCACAACCTTTGCCGTGACCAGCATTCTGAGAGTGGCCGCCGAGGATTGGAAGAAGGGCGA
CACCTTTAGCTGCATGGTCGGACACGAAGCGCTGCCCCTGGCTTTCACCCAGAAAACCATCGATAGACTGGCC
GGC
SEQ ID NO: 62
BDR5-1HF H chain (Protein) (VH ONLY FROM HC-hDR5-01-G56T
SEQ ID NO: 36 of U.S. Pat. No. 10,882,913)
EVQLQQSGAEVVKPGASVKLSCKASGFNIKDTFIHWVKQAPGQGLEWIGRIDPANTNTKYDPKFQGKATITTD
TSSNTAYMELSSLRSEDTAVYYCVRGLYTYYFDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKRVEPK
SCDKTHTCPPCPAPELLGGPSVFLFPPKPKDTLMISRTPEVTCVVVDVSHEDPEVKENWYVDGVEVHNAKTKP
REEQYNSTYRVVSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMFEALHNHYT
QKSLSLSPG
SEQ ID NO: 63
BDR5-1HF heavy chain (DNA)
GAAGTGCAGCTGCAACAGTCTGGCGCCGAGGTTGTGAAACCTGGCGCCTCTGTGAAGCTGAGCTGTAAAGCCA
GCGGCTTCAACATCAAGGACACCTTCATCCACTGGGTCAAGCAGGCCCCTGGACAGGGACTCGAATGGATCGG
CAGAATCGACCCCGCCAACACCAACACTAAGTACGACCCCAAGTTCCAGGGCAAAGCCACCATCACCACCGAC
ACCAGCAGCAACACCGCCTACATGGAACTGAGCAGCCTGAGAAGCGAGGACACCGCCGTGTACTATTGTGTGC
GGGGCCTGTACACCTACTACTTCGATTATTGGGGCCAGGGTACCCTGGTCACAGTTAGCTCTGCTAGCACAAA
AGGCCCTAGCGTCTTTCCACTCGCCCCATCCTCCAAGAGCACCTCTGGGGGCACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAGCTTGGG
CACCCAGACCTATATCTGCAATGTCAATCATAAACCCAGTAACACAAAGGTAGACAAGAGAGTCGAACCCAAA
TCTTGCGACAAAACTCACACATGCCCTCCTTGCCCCGCCCCCGAACTCCTGGGGGGACCTTCGGTTTTTCTTT
TTCCACCTAAACCGAAAGACACCCTGATGATCAGCAGGACACCAGAAGTGACATGTGTCGTCGTGGACGTATC
CCATGAGGACCCGGAGGTGAAGTTCAACTGGTATGTTGATGGTGTGGAAGTGCATAATGCAAAAACTAAGCCA
CGGGAGGAACAGTACAATAGCACATACAGAGTAGTTAGCGTTCTGACTGTCCTGCACCAAGATTGGCTGAACG
GGAAGGAATACAAGTGTAAGGTGAGCAACAAAGCCCTGCCCGCTCCAATTGAGAAAACAATTTCTAAGGCCAA
AGGACAGCCCCGGGAGCCACAGGTGTATACTCTGCCGCCTAGCAGGGAGGAAATGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCGTGCTTGATAGCGACGGATCATTTTTCCTGTACTCAAAACTGACCGTGGA
CAAAAGCAGATGGCAGCAGGGAAACGTTTTCAGTTGCTCAGTGATGTTCGAAGCGCTGCACAATCATTATACT
CAGAAAAGCCTGAGCTTGAGCCCGGGA
SEQ ID NO: 64
BDR5-2HF H chain (Protein) (VH ONLY FROM HC-hDR5-01-G56T
SEQ ID NO: 36 of U.S. Pat. No. 10,882,913)
EVQLQQSGAEVVKPGASVKLSCKASGFNIKDTFIHWVKQAPGQGLEWIGRIDPANTNTKYDPKFQGKATITTD
TSSNTAYMELSSLRSEDTAVYYCVRGLYTYYFDYWGQGTLVTVSSASTKGPSVFPLAPCSRSTSESTAALGCL
VKDYFPEPVTVSWNSGALTSGVHTFPAVLQSSGLYSLSSVVTVPSSNFGTQTYTCNVDHKPSNTKVDKTVERK
CCVECPPCPAPPVAGPSVFLFPPKPKDTLMISRTPEVTCVVVDVSHEDPEVQFNWYVDGVEVHNAKTKPREEQ
FNSTFRVVSVLTVVHQDWLNGKEYKCKVSNKGLPAPIEKTISKTKGQPREPQVYTLPPSREEMTKNQVSLTCL
VKGFYPSDIAVEWESNGQPENNYKTTPPMLDSDGSFFLYSKLTVDKSRWQQGNVFSCSVMFEALHNHYTQKSL
SLSPG
SEQ ID NO: 65
BDR5-2HF heavy chain (DNA)
GAAGTGCAGCTGCAACAGTCTGGCGCCGAGGTTGTGAAACCTGGCGCCTCTGTGAAGCTGAGCTGTAAAGCCA
GCGGCTTCAACATCAAGGACACCTTCATCCACTGGGTCAAGCAGGCCCCTGGACAGGGACTCGAATGGATCGG
CAGAATCGACCCCGCCAACACCAACACTAAGTACGACCCCAAGTTCCAGGGCAAAGCCACCATCACCACCGAC
ACCAGCAGCAACACCGCCTACATGGAACTGAGCAGCCTGAGAAGCGAGGACACCGCCGTGTACTATTGTGTGC
GGGGCCTGTACACCTACTACTTCGACTATTGGGGCCAGGGTACCCTGGTCACAGTTAGCTCTGCTAGCACAAA
AGGCCCTAGCGTCTTTCCACTCGCCCCATGTTCAAGAAGCACCAGCGAGTCAACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAATTTTGG
CACCCAGACCTATACATGCAATGTCGATCATAAACCCAGTAACACAAAGGTAGACAAGACAGTCGAAAGAAAA
TGCTGTGTGGAGTGCCCTCCTTGCCCCGCCCCCCCAGTGGCCGGGCCTTCGGTTTTTCTTTTTCCACCTAAAC
CGAAAGACACCCTGATGATCAGCAGGACACCAGAAGTGACATGTGTCGTCGTGGACGTATCCCATGAAGACCC
GGAGGTGCAATTCAACTGGTATGTTGATGGTGTGGAAGTGCATAATGCAAAAACTAAGCCACGGGAGGAACAG
TTCAATAGCACATTCAGAGTAGTTAGCGTTCTGACTGTCGTGCACCAAGATTGGCTGAACGGGAAGGAATACA
AGTGTAAGGTGAGCAACAAAGGTCTGCCCGCTCCAATTGAGAAAACAATTTCTAAGACCAAAGGACAGCCCCG
GGAGCCACAGGTGTATACTCTGCCGCCTAGCAGGGAGGAAATGACCAAGAACCAGGTGAGCCTGACCTGTCTG
GTGAAAGGCTTCTACCCCAGCGATATCGCAGTTGAGTGGGAGAGCAATGGCCAACCCGAGAACAACTACAAGA
CTACGCCCCCCATGCTTGATAGCGACGGATCATTTTTCCTGTACTCAAAACTGACCGTGGACAAAAGCAGATG
GCAGCAGGGAAACGTTTTCAGTTGCTCAGTGATGTTCGAAGCGCTGCACAATCATTATACTCAGAAAAGCCTG
AGCTTGAGCCCGGGA
SEQ ID NO: 66
TDR5-1HF heavy chain (protein) (HC-hDR5-05 (SEQ ID NO: 40
of U.S. Pat. No. 10,882,913)
QVQLVQSGAEVKKPGASVKVSCKASGFNIKDTHMHWVRQAPGQRLEWIGRIDPANGNTEYDQKFQGRVTITVD
TSASTAYMELSSLRSEDTAVYYCARWGTNVYFAYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKRVEPK
SCDKTHTCPPCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMFEALHNHYT
QKSLSLSPG
SEQ ID NO: 67
TDR5-1HF heavy chain (DNA)
CAGGTTCAGCTGGTTCAGTCTGGCGCCGAAGTGAAGAAACCTGGCGCCTCTGTGAAGGTGTCCTGCAAGGCCA
GCGGCTTCAACATCAAGGACACCCACATGCACTGGGTCCGACAGGCTCCAGGACAGAGACTGGAATGGATCGG
CAGAATCGACCCCGCCAACGGCAACACCGAGTACGACCAGAAATTCCAGGGCAGAGTGACCATCACCGTGGAC
ACATCTGCCAGCACCGCCTACATGGAACTGAGCAGCCTGAGAAGCGAGGACACCGCCGTGTACTACTGTGCCA
GATGGGGCACCAACGTGTACTTCGCCTATTGGGGCCAGGGTACCCTGGTCACAGTTAGCTCTGCTAGCACAAA
AGGCCCTAGCGTCTTTCCACTCGCCCCATCCTCCAAGAGCACCTCTGGGGGCACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAGCTTGGG
CACCCAGACCTATATCTGCAATGTCAATCATAAACCCAGTAACACAAAGGTAGACAAGAGAGTCGAACCCAAA
TCTTGCGACAAAACTCACACATGCCCTCCTTGCCCCGCCCCCGAACTCCTGGGGGGACCTTCGGTTTTTCTTT
TTCCACCTAAACCGAAAGACACCCTGATGATCAGCAGGACACCAGAAGTGACATGTGTCGTCGTGGACGTATC
CCATGAGGACCCGGAGGTGAAGTTCAACTGGTATGTTGATGGTGTGGAAGTGCATAATGCAAAAACTAAGCCA
CGGGAGGAACAGTACAATAGCACATACAGAGTAGTTAGCGTTCTGACTGTCCTGCACCAAGATTGGCTGAACG
GGAAGGAATACAAGTGTAAGGTGAGCAACAAAGCCCTGCCCGCTCCAATTGAGAAAACAATTTCTAAGGCCAA
AGGACAGCCCCGGGAGCCACAGGTGTATACTCTGCCGCCTAGCAGGGAGGAAATGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCGTGCTTGATAGCGACGGATCATTTTTCCTGTACTCAAAACTGACCGTGGA
CAAAAGCAGATGGCAGCAGGGAAACGTTTTCAGTTGCTCAGTGATGTTCGAAGCGCTGCACAATCATTATACT
CAGAAAAGCCTGAGCTTGAGCCCGGGA
SEQ ID NO: 68
TDR5-2HF heavy chain (protein) (HC-hDR5-05 (SEQ ID NO:
40 of U.S. Pat. No. 10,882,913)
QVQLVQSGAEVKKPGASVKVSCKASGFNIKDTHMHWVRQAPGQRLEWIGRIDPANGNTEYDQKFQGRVTITVD
TSASTAYMELSSLRSEDTAVYYCARWGTNVYFAYWGQGTLVTVSSASTKGPSVFPLAPCSRSTSESTAALGCL
VKDYFPEPVTVSWNSGALTSGVHTFPAVLQSSGLYSLSSVVTVPSSNFGTQTYTCNVDHKPSNTKVDKTVERK
CCVECPPCPAPPVAGPSVFLFPPKPKDTLMISRTPEVTCVVVDVSHEDPEVQFNWYVDGVEVHNAKTKPREEQ
FNSTFRVVSVLTVVHQDWLNGKEYKCKVSNKGLPAPIEKTISKTKGQPREPQVYTLPPSREEMTKNQVSLTCL
VKGFYPSDIAVEWESNGQPENNYKTTPPMLDSDGSFFLYSKLTVDKSRWQQGNVFSCSVMFEALHNHYTQKSL
SLSPG
SEQ ID NO: 69
TDR5-2HF heavy chain (DNA)
CAGGTTCAGCTGGTTCAGTCTGGCGCCGAAGTGAAGAAACCTGGCGCCTCTGTGAAGGTGTCCTGCAAGGCCA
GCGGCTTCAACATCAAGGACACCCACATGCACTGGGTCCGACAGGCTCCAGGACAGAGACTGGAATGGATCGG
CAGAATCGACCCCGCCAACGGCAACACCGAGTACGACCAGAAATTCCAGGGCAGAGTGACCATCACCGTGGAC
ACATCTGCCAGCACCGCCTACATGGAACTGAGCAGCCTGAGAAGCGAGGACACCGCCGTGTACTACTGTGCCA
GATGGGGCACCAACGTGTACTTCGCCTATTGGGGACAGGGTACCCTCGTGACCGTTAGCTCTGCTAGCACAAA
AGGCCCTAGCGTCTTTCCACTCGCCCCATGTTCAAGAAGCACCAGCGAGTCAACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAATTTTGG
CACCCAGACCTATACATGCAATGTCGATCATAAACCCAGTAACACAAAGGTAGACAAGACAGTCGAAAGAAAA
TGCTGTGTGGAGTGCCCTCCTTGCCCCGCCCCCCCAGTGGCCGGGCCTTCGGTTTTTCTTTTTCCACCTAAAC
CGAAAGACACCCTGATGATCAGCAGGACACCAGAAGTGACATGTGTCGTCGTGGACGTATCCCATGAAGACCC
GGAGGTGCAATTCAACTGGTATGTTGATGGTGTGGAAGTGCATAATGCAAAAACTAAGCCACGGGAGGAACAG
TTCAATAGCACATTCAGAGTAGTTAGCGTTCTGACTGTCGTGCACCAAGATTGGCTGAACGGGAAGGAATACA
AGTGTAAGGTGAGCAACAAAGGTCTGCCCGCTCCAATTGAGAAAACAATTTCTAAGACCAAAGGACAGCCCCG
GGAGCCACAGGTGTATACTCTGCCGCCTAGCAGGGAGGAAATGACCAAGAACCAGGTGAGCCTGACCTGTCTG
GTGAAAGGCTTCTACCCCAGCGATATCGCAGTTGAGTGGGAGAGCAATGGCCAACCCGAGAACAACTACAAGA
CTACGCCCCCCATGCTTGATAGCGACGGATCATTTTTCCTGTACTCAAAACTGACCGTGGACAAAAGCAGATG
GCAGCAGGGAAACGTTTTCAGTTGCTCAGTGATGTTCGAAGCGCTGCACAATCATTATACTCAGAAAAGCCTG
AGCTTGAGCCCGGGA
SEQ ID NO: 70 BDR5-A2HF H chain (Protein) (VH ONLY FROM HC-hDR5-01-G56T
SEQ ID NO: 36 of U.S. Pat. No. 10,882,913)
EVQLQQSGAEVVKPGASVKLSCKASGFNIKDTFIHWVKQAPGQGLEWIGRIDPANTNTKYDPKFQGKATITTD
TSSNTAYMELSSLRSEDTAVYYCVRGLYTYYFDYWGQGTLVTVSSASPTSPKVFPLSLDSTPQDGNVVVACLV
QGFFPQEPLSVTWSESGQNVTARNFPPSQDASGDLYTTSSQLTLPATQCPDGKSVTCHVKHYTNPSQDVTVPC
PVPPPPPCCHPRLSLHRPALEDLLLGSEANLTCTLTGLRDASGATFTWTPSSGKSAVQGPPERDLCGCYSVSS
VLPGCAQPWNHGETFTCTAAHPELKTPLTANITKSGNTFRPEVHLLPPPSEELALNELVTLTCLARGESPKDV
LVRWLQGSQELPREKYLTWASRQEPSQGTTTFAVTSILRVAAEDWKKGDTFSCMVGFEALPLAFTQKTIDRLA
G
SEQ ID NO: 71
BDR5-A2HF heavy chain (DNA)
GAAGTTCAGCTGCAACAGTCTGGCGCCGAGGTTGTGAAACCTGGCGCCTCTGTGAAGCTGAGCTGTAAAGCCA
GCGGCTTCAACATCAAGGACACCTTCATCCACTGGGTCAAGCAGGCCCCTGGACAGGGACTCGAGTGGATCGG
AAGAATCGACCCCGCCAACACCAACACTAAGTACGACCCCAAGTTCCAGGGCAAAGCCACCATCACCACCGAC
ACCAGCAGCAACACCGCCTACATGGAACTGAGCAGCCTGAGAAGCGAGGACACCGCCGTGTACTATTGTGTGC
GGGGCCTGTACACCTACTACTTCGACTATTGGGGCCAGGGTACCCTCGTGACCGTTAGCTCTGCTAGCCCTAC
AAGCCCCAAGGTGTTCCCTCTGAGCCTGGATAGCACACCCCAGGACGGAAATGTGGTGGTGGCTTGTCTGGTG
CAGGGATTCTTCCCACAAGAGCCCCTGTCCGTGACTTGGAGCGAGAGCGGACAGAATGTGACCGCCAGAAACT
TCCCACCTAGCCAGGATGCCAGCGGCGATCTGTACACAACAAGCAGCCAGCTGACCCTGCCTGCCACACAGTG
TCCTGATGGCAAGAGCGTGACCTGCCACGTGAAGCACTACACAAACCCCAGCCAGGACGTGACCGTGCCTTGT
CCTGTTCCTCCTCCACCTCCATGCTGTCACCCTAGACTGAGCCTGCACAGACCCGCTCTGGAAGATCTGCTGC
TGGGCTCTGAGGCCAACCTGACCTGTACACTGACCGGCCTGAGAGATGCCTCTGGCGCCACCTTTACATGGAC
ACCTAGCAGCGGAAAGTCCGCCGTTCAGGGACCTCCTGAGAGGGATCTGTGTGGCTGTTACAGCGTGTCCTCT
GTGCTGCCTGGATGTGCCCAGCCTTGGAATCACGGCGAGACATTCACCTGTACCGCCGCTCATCCCGAGCTGA
AAACACCCCTGACCGCCAACATCACCAAGAGCGGCAATACCTTCAGACCCGAAGTGCATCTGCTGCCTCCACC
ATCTGAAGAACTGGCCCTGAACGAGCTGGTGACCCTGACATGTCTGGCCAGGGGCTTCAGCCCTAAGGATGTG
CTCGTTAGATGGCTGCAGGGCAGCCAAGAGCTGCCCAGAGAGAAGTATCTGACCTGGGCCAGTCGACAGGAAC
CTTCTCAGGGCACCACAACCTTTGCCGTGACCAGCATTCTGAGAGTGGCCGCCGAGGATTGGAAGAAGGGCGA
CACCTTTAGCTGCATGGTCGGATTCGAAGCGCTGCCCCTGGCTTTCACCCAGAAAACCATCGATAGACTGGCC
GGC
SEQ ID NO: 72
TDR5-A2HF heavy chain (protein) (HC-hDR5-05 (SEQ ID NO:
40 of U.S. Pat. No. 10,882,913)
QVQLVQSGAEVKKPGASVKVSCKASGFNIKDTHMHWVRQAPGQRLEWIGRIDPANGNTEYDQKFQGRVTITVD
TSASTAYMELSSLRSEDTAVYYCARWGTNVYFAYWGQGTLVTVSSASPTSPKVFPLSLDSTPQDGNVVVACLV
QGFFPQEPLSVTWSESGQNVTARNFPPSQDASGDLYTTSSQLTLPATQCPDGKSVTCHVKHYTNPSQDVTVPC
PVPPPPPCCHPRLSLHRPALEDLLLGSEANLTCTLTGLRDASGATFTWTPSSGKSAVQGPPERDLCGCYSVSS
VLPGCAQPWNHGETFTCTAAHPELKTPLTANITKSGNTFRPEVHLLPPPSEELALNELVTLTCLARGESPKDV
LVRWLQGSQELPREKYLTWASRQEPSQGTTTFAVTSILRVAAEDWKKGDTFSCMVGFEALPLAFTQKTIDRLA
G
SEQ ID NO: 73
TDR5-A2HF heavy chain (DNA)
CAGGTTCAGCTGGTTCAGTCTGGCGCCGAAGTGAAGAAACCTGGCGCCTCTGTGAAGGTGTCCTGCAAGGCCA
GCGGCTTCAACATCAAGGACACCCACATGCACTGGGTCCGACAGGCTCCAGGACAGAGACTGGAATGGATCGG
CAGAATCGACCCCGCCAACGGCAACACCGAGTACGACCAGAAATTCCAGGGCAGAGTGACCATCACCGTGGAC
ACATCTGCCAGCACCGCCTACATGGAACTGAGCAGCCTGAGAAGCGAGGACACCGCCGTGTACTACTGTGCCA
GATGGGGCACCAACGTGTACTTCGCCTATTGGGGACAGGGTACCCTCGTGACCGTTAGCTCTGCTAGCCCTAC
AAGCCCCAAGGTGTTCCCTCTGAGCCTGGATAGCACACCCCAGGACGGAAATGTGGTGGTGGCTTGTCTGGTG
CAGGGATTCTTCCCACAAGAGCCCCTGTCCGTGACTTGGAGCGAGAGCGGACAGAATGTGACCGCCAGAAACT
TCCCACCTAGCCAGGATGCCAGCGGCGATCTGTACACAACAAGCAGCCAGCTGACCCTGCCTGCCACACAGTG
TCCTGATGGCAAGAGCGTGACCTGCCACGTGAAGCACTACACAAACCCCAGCCAGGACGTGACCGTGCCTTGT
CCTGTTCCTCCTCCACCTCCATGCTGTCACCCTAGACTGAGCCTGCACAGACCCGCTCTGGAAGATCTGCTGC
TGGGCTCTGAGGCCAACCTGACCTGTACACTGACCGGCCTGAGAGATGCCTCTGGCGCCACCTTTACATGGAC
ACCTAGCAGCGGAAAGTCCGCCGTTCAGGGACCTCCTGAGAGGGATCTGTGTGGCTGTTACAGCGTGTCCTCT
GTGCTGCCTGGATGTGCCCAGCCTTGGAATCACGGCGAGACATTCACCTGTACCGCCGCTCATCCCGAGCTGA
AAACACCCCTGACCGCCAACATCACCAAGAGCGGCAATACCTTCAGACCCGAAGTGCATCTGCTGCCTCCACC
ATCTGAAGAACTGGCCCTGAACGAGCTGGTGACCCTGACATGTCTGGCCAGGGGCTTCAGCCCTAAGGATGTG
CTCGTTAGATGGCTGCAGGGCAGCCAAGAGCTGCCCAGAGAGAAGTATCTGACCTGGGCCAGTCGACAGGAAC
CTTCTCAGGGCACCACAACCTTTGCCGTGACCAGCATTCTGAGAGTGGCCGCCGAGGATTGGAAGAAGGGCGA
CACCTTTAGCTGCATGGTCGGATTCGAAGCGCTGCCCCTGGCTTTCACCCAGAAAACCATCGATAGACTGGCC
GGC
SEQ ID NO: 74
mezagitamab (CD38) WT heavy chain amino acid sequence
[Sequence 21 from U.S. Pat. No. 8,362,211]
EVQLLESGGGLVQPGGSLRLSCAASGFTEDDYGMSWVRQAPGKGLEWVSDISWNGGKTHYVDSVKGQFTISRD
NSKNTLYLQMNSLRAEDTAVYYCARGSLFHDSSGFYFGHWGQGTLVTVSSASTKGPSVFPLAPSSKSTSGGTA
ALGCLVKDYFPEPVTVSWNSGALTSGVHTFPAVLQSSGLYSLSSVVTVPSSSLGTQTYICNVNHKPSNTKVDK
RVEPKSCDKTHTCPPCPAPELLGGPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTISKAKGQPREPQVYTLPPSREEMT
KNQVSLTCLVKGFYPSDIAVEWESNGQPENNYKTTPPVLDSDGSFFLYSKLTVDKSRWQQGNVFSCSVMHEAL
HNHYTQKSLSLSPGK
SEQ ID NO: 75
mezagitamab (CD38) WT heavy chain nucleotide sequence
GAAGTGCAGCTGCTGGAATCTGGCGGCGGACTTGTTCAACCAGGCGGCTCTCTGAGACTGAGCTGTGCCGCTT
CCGGCTTCACCTTCGACGATTACGGCATGAGCTGGGTCCGACAGGCCCCTGGAAAAGGCCTTGAATGGGTGTC
CGACATCAGCTGGAATGGCGGCAAGACCCACTACGTGGACTCTGTGAAGGGCCAGTTCACCATCAGCCGGGAC
AACAGCAAGAACACCCTGTACCTGCAGATGAACAGCCTGAGAGCCGAGGACACCGCCGTGTACTATTGTGCCA
GAGGCAGCCTGTTCCACGACAGCAGCGGCTTCTATTTTGGCCACTGGGGCCAGGGCACACTGGTCACAGTTAG
CTCTGCTAGCACAAAAGGCCCTAGCGTCTTTCCACTCGCCCCATCCTCCAAGAGCACCTCTGGGGGCACTGCG
GCTTTGGGCTGCTTGGTGAAGGATTACTTCCCAGAGCCTGTCACTGTCAGCTGGAATAGCGGGGCTCTGACCA
GTGGAGTGCACACCTTTCCCGCCGTGTTACAGAGCAGCGGCCTCTATAGCCTGAGCAGTGTGGTAACTGTGCC
CTCGAGCAGCTTGGGTACCCAGACCTATATCTGCAATGTCAATCATAAACCCAGTAACACAAAGGTAGACAAG
AGAGTCGAACCCAAATCTTGCGACAAAACTCACACATGCCCTCCTTGCCCCGCCCCCGAACTCCTGGGGGGAC
CTTCGGTTTTTCTTTTTCCACCTAAACCGAAAGACACCCTGATGATCAGCAGGACACCAGAAGTGACATGTGT
CGTCGTGGACGTATCCCATGAGGACCCGGAGGTGAAGTTCAACTGGTATGTTGATGGTGTGGAAGTGCATAAT
GCAAAAACTAAGCCACGGGAGGAACAGTACAATAGCACATACAGAGTAGTTAGCGTTCTGACTGTCCTGCACC
AAGATTGGCTGAACGGGAAGGAATACAAGTGTAAGGTGAGCAACAAAGCCCTGCCCGCTCCAATTGAGAAAAC
AATTTCTAAGGCCAAAGGACAGCCCCGGGAGCCACAGGTGTATACTCTGCCGCCTAGCAGGGAGGAAATGACC
AAGAACCAGGTGAGCCTGACCTGTCTGGTGAAAGGCTTCTACCCCAGCGATATCGCAGTTGAGTGGGAGAGCA
ATGGCCAACCCGAGAACAACTACAAGACTACGCCCCCCGTGCTTGATAGCGACGGATCATTTTTCCTGTACTC
AAAACTGACCGTGGACAAAAGCAGATGGCAGCAGGGAAACGTTTTCAGTTGCTCAGTGATGCACGAAGCGCTG
CACAATCATTATACTCAGAAAAGCCTGAGCTTGAGCCCGGGAAAA
SEQ ID NO: 76
mezagitamab (CD38) light chain amino acid sequence [Sequence
22 from U.S. Pat. No. 8,362,211]
QSVLTQPPSASGTPGQRVTISCSGSSSNIGDNYVSWYQQLPGTAPKLLIYRDSQRPSGVPDRFSGSKSGTSAS
LAISGLRSEDEADYYCQSYDSSLSGSVFGGGTKLTVLGQPKANPTVTLFPPSSEELQANKATLVCLISDFYPG
AVTVAWKADGSPVKAGVETTKPSKQSNNKYAASSYLSLTPEQWKSHRSYSCQVTHEGSTVEKTVAPTECS
SEQ ID NO: 77
mezagitamab (CD38) light chain nucleotide sequence
CAATCTGTTCTGACACAGCCTCCTAGCGCCTCTGGCACACCTGGACAGAGAGTGACCATCAGCTGTAGCGGCA
GCAGCAGCAACATCGGCGACAACTACGTGTCCTGGTATCAGCAGCTGCCTGGCACAGCCCCTAAACTGCTGAT
CTACCGGGATAGCCAGAGGCCAAGCGGCGTGCCAGATAGATTTTCCGGCAGCAAGAGCGGCACATCTGCCAGC
CTGGCTATCAGCGGACTGAGATCTGAGGACGAGGCCGACTACTACTGCCAGAGCTACGATAGCAGCCTGAGCG
GCTCTGTGTTTGGCGGAGGTACCAAGCTGACCGTGCTGGGCCAACCTAAAGCCAATCCTACCGTGACACTGTT
CCCTCCAAGCAGCGAAGAACTGCAGGCCAACAAGGCCACACTCGTGTGCCTGATCAGCGACTTTTATCCTGGC
GCCGTGACCGTGGCCTGGAAGGCTGATGGATCTCCTGTGAAAGCCGGCGTGGAAACCACCAAGCCTAGCAAGC
AGAGCAACAACAAATACGCCGCCAGCAGCTACCTGAGCCTGACACCTGAGCAGTGGAAGTCCCACAGATCCTA
CAGCTGCCAAGTGACCCACGAGGGCAGCACCGTGGAAAAGACAGTGGCCCCTACCGAGTGCAGC
SEQ ID NO: 78
S2P6 IgG1 WT H chain amino acid sequence
EVQLVQSGAEVKKPGASVKVSCKASGYTFTSQYMHWVRQAPGQGLEWIGIINPSGVHTSYAQKFQGRVTLTRD
TSTSTLYMELSSLRSEDTAVYYCARGSPKGAFDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMHEALHNHYT
QKSLSLSPGK
SEQ ID NO: 79
S2P6 IgG1 WT H chain nucleotide sequence
GAAGTTCAGCTGGTTCAGTCTGGCGCCGAAGTGAAGAAACCTGGCGCCTCTGTGAAGGTGTCCTGCAAGGCCA
GCGGCTACACCTTTACCAGCCAGTACATGCACTGGGTCCGACAGGCTCCAGGACAGGGACTCGAGTGGATCGG
CATCATCAATCCTAGCGGCGTGCACACCAGCTACGCCCAGAAATTCCAGGGCAGAGTGACCCTGACCAGAGAC
ACCAGCACCAGCACACTGTACATGGAACTGAGCAGCCTGCGGAGCGAGGATACCGCCGTGTACTATTGTGCCA
GAGGCTCTCCTAAGGGCGCCTTCGATTATTGGGGCCAGGGCACACTGGTCACCGTGTCCTCTGCTAGCACAAA
AGGCCCTAGCGTCTTTCCACTCGCCCCATCCTCCAAGAGCACCTCTGGGGGCACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAGCTTGGG
TACCCAGACCTATATCTGCAATGTCAATCATAAACCCAGTAACACAAAGGTAGACAAGAAAGTCGAACCCAAA
TCTTGCGACAAAACTCACACATGCCCTCCTTGCCCCGCCCCCGAACTCCTGGGGGGACCTTCGGTTTTTCTTT
TTCCACCTAAACCGAAAGACACCCTGATGATCAGCAGGACACCAGAAGTGACATGTGTCGTCGTGGACGTATC
CCATGAGGACCCGGAGGTGAAGTTCAACTGGTATGTTGATGGTGTGGAAGTGCATAATGCAAAAACTAAGCCA
CGGGAGGAACAGTACAATAGCACATACAGAGTAGTTAGCGTTCTGACTGTCCTGCACCAAGATTGGCTGAACG
GGAAGGAATACAAGTGTAAGGTGAGCAACAAAGCCCTGCCCGCTCCAATTGAGAAAACAATTTCTAAGGCCAA
AGGACAGCCCCGGGAGCCACAGGTGTATACTCTGCCGCCTAGCAGGGAGGAAATGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCGTGCTTGATAGCGACGGATCATTTTTCCTGTACTCAAAACTGACCGTGGA
CAAAAGCAGATGGCAGCAGGGAAACGTTTTCAGTTGCTCAGTGATGCACGAAGCGCTGCACAATCATTATACT
CAGAAAAGCCTGAGCTTGAGCCCGGGAAAA
SEQ ID NO: 80
S2P6 kappa light chain amino acid sequence
EIVMMQSPGTLSLSPGERATLSCRASQSVRSNYLAWYQQKPGQAPRLLIYGASSRATGIPDRESGSGSGTDET
LTISRLEPEDFAVYYCQQYGSSPPRFTFGPGTKVEIKRTVAAPSVFIFPPSDEQLKSGTASVVCLLNNFYPRE
AKVQWKVDNALQSGNSQESVTEQDSKDSTYSLSSTLTLSKADYEKHKVYACEVTHQGLSSPVTKSENRGEC
SEQ ID NO: 81
S2P6 kappa light chain nucleotide sequence
GAGATCGTCATGATGCAGAGCCCCGGCACACTGTCACTGTCTCCAGGCGAAAGAGCCACACTGAGCTGTAGAG
CCTCTCAGAGCGTGCGGAGCAATTACCTGGCCTGGTATCAGCAGAAGCCCGGACAGGCTCCTCGGCTGTTGAT
CTATGGCGCCTCTAGCAGAGCCACTGGCATCCCCGATAGATTTTCTGGCAGCGGCTCCGGCACCGACTTCACC
CTGACAATCAGCAGACTGGAACCCGAGGACTTCGCCGTGTACTACTGTCAGCAGTACGGCAGCAGCCCTCCTC
GGTTCACATTTGGCCCTGGTACCAAGGTGGAAATCAAGCGGACAGTGGCCGCTCCTAGCGTGTTCATCTTTCC
ACCTAGCGACGAGCAGCTGAAGTCTGGCACAGCCTCTGTCGTGTGCCTGCTGAACAACTTCTACCCCAGAGAA
GCCAAGGTGCAGTGGAAGGTGGACAACGCCCTGCAGAGCGGCAATAGCCAAGAGAGCGTGACCGAGCAGGACA
GCAAGGACTCTACCTACAGCCTGAGCAGCACCCTGACACTGAGCAAGGCCGACTACGAGAAGCACAAAGTGTA
CGCCTGCGAAGTGACCCACCAGGGCCTTTCTAGCCCTGTGACCAAGAGCTTCAACCGGGGCGAGTGC
SEQ ID NO: 82
CC40.8 IgG1 WT H Chain amino acid sequence
QVQEVQLLESGGGLVQPGGSLRLSCAASGFTFSSYVMTWARQAPGKGLEWVSAISGTGYTYYADSVKGRFTVS
RDNSKNTLFLQMSSLRAEDTAVYYCAITMAPVVWGQGTTVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKENWYVDGVEVHNAKTKPR
EEQYNSTYRVVSVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSVMHEALHNHYTQ
KSLSLSPGK
SEQ ID NO: 83
CV3-25 WT H chain amino acid sequence
QVQLVESGAEVKKPGESLKISCKGSGYTFTRYWIGWVRQMPGKGLEWMGIIYPGDSDTRYSPSFQGHVTISAD
KSISTAYLQWNSLKASDTAMYYCARLPQYCSNGVCQRWFDPWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHEDPEVKENWYVDGVEV
HNAKTKPREEQYNSTYRVVSVLTVLHQDWLNGKEYKCKVSNKALPAPIEKTISKAKGQPREPQVYTLPPSREE
MTKNQVSLTCLVKGFYPSDIAVEWESNGQPENNYKTTPPVLDSDGSFFLYSKLTVDKSRWQQGNVFSCSVMHE
ALHNHYTQKSLSLSPGK
SEQ ID NO: 84
CC40.8 IgG1 WT H chain nucleotide sequence
CAAGTGCAGGAAGTGCAGCTGCTGGAATCTGGCGGAGGACTGGTTCAACCAGGCGGCTCTCTGAGACTGTCTT
GTGCCGCCAGCGGCTTCACCTTCAGCAGCTACGTTATGACCTGGGCCAGACAGGCCCCTGGCAAAGGACTTGA
ATGGGTGTCCGCCATCAGCGGCACAGGCTACACCTACTACGCCGACTCTGTGAAGGGCAGATTCACCGTGTCC
AGAGACAACAGCAAGAATACCCTGTTCCTGCAGATGAGCAGCCTGAGAGCCGAGGATACCGCCGTGTACTACT
GCGCCATTACAATGGCCCCTGTCGTGTGGGGCCAGGGCACAACAGTGACAGTCAGCAGCGCTAGCACAAAAGG
CCCTAGCGTCTTTCCACTCGCCCCATCCTCCAAGAGCACCTCTGGGGGCACTGCGGCTTTGGGCTGCTTGGTG
AAGGATTACTTCCCAGAGCCTGTCACTGTCAGCTGGAATAGCGGGGCTCTGACCAGTGGAGTGCACACCTTTC
CCGCCGTGTTACAGAGCAGCGGCCTCTATAGCCTGAGCAGTGTGGTAACTGTGCCCTCGAGCAGCTTGGGTAC
CCAGACCTATATCTGCAATGTCAATCATAAACCCAGTAACACAAAGGTAGACAAGAAAGTCGAACCCAAATCT
TGCGACAAAACTCACACATGCCCTCCTTGCCCCGCCCCCGAACTCCTGGGGGGACCTTCGGTTTTTCTTTTTC
CACCTAAACCGAAAGACACCCTGATGATCAGCAGGACACCAGAAGTGACATGTGTCGTCGTGGACGTATCCCA
TGAGGACCCGGAGGTGAAGTTCAACTGGTATGTTGATGGTGTGGAAGTGCATAATGCAAAAACTAAGCCACGG
GAGGAACAGTACAATAGCACATACAGAGTAGTTAGCGTTCTGACTGTCCTGCACCAAGATTGGCTGAACGGGA
AGGAATACAAGTGTAAGGTGAGCAACAAAGCCCTGCCCGCTCCAATTGAGAAAACAATTTCTAAGGCCAAAGG
ACAGCCCCGGGAGCCACAGGTGTATACTCTGCCGCCTAGCAGGGAGGAAATGACCAAGAACCAGGTGAGCCTG
ACCTGTCTGGTGAAAGGCTTCTACCCCAGCGATATCGCAGTTGAGTGGGAGAGCAATGGCCAACCCGAGAACA
ACTACAAGACTACGCCCCCCGTGCTTGATAGCGACGGATCATTTTTCCTGTACTCAAAACTGACCGTGGACAA
AAGCAGATGGCAGCAGGGAAACGTTTTCAGTTGCTCAGTGATGCACGAAGCGCTGCACAATCATTATACTCAG
AAAAGCCTGAGCTTGAGCCCGGGAAAA
SEQ ID NO: 85
CV3-25 WT H chain nucleotide sequence
CAAGTGCAGCTGGTTGAATCTGGCGCCGAAGTGAAGAAGCCTGGCGAGAGCCTGAAGATCAGCTGCAAAGGCA
GCGGCTACACCTTCACCAGATACTGGATCGGCTGGGTCCGACAGATGCCTGGCAAAGGCCTTGAGTGGATGGG
CATCATCTACCCCGGCGACAGCGATACCAGATACAGCCCTAGCTTTCAGGGCCACGTGACCATCAGCGCCGAC
AAGTCTATCAGCACCGCCTACCTGCAGTGGAACAGCCTGAAGGCCAGCGACACCGCCATGTACTACTGTGCCA
GACTGCCCCAGTACTGCAGCAATGGCGTTTGCCAGCGTTGGTTCGATCCTTGGGGACAGGGCACACTGGTCAC
CGTTAGCAGCGCTAGCACAAAAGGCCCTAGCGTCTTTCCACTCGCCCCATCCTCCAAGAGCACCTCTGGGGGC
ACTGCGGCTTTGGGCTGCTTGGTGAAGGATTACTTCCCAGAGCCTGTCACTGTCAGCTGGAATAGCGGGGCTC
TGACCAGTGGAGTGCACACCTTTCCCGCCGTGTTACAGAGCAGCGGCCTCTATAGCCTGAGCAGTGTGGTAAC
TGTGCCCTCGAGCAGCTTGGGTACCCAGACCTATATCTGCAATGTCAATCATAAACCCAGTAACACAAAGGTA
GACAAGAAAGTCGAACCCAAATCTTGCGACAAAACTCACACATGCCCTCCTTGCCCCGCCCCCGAACTCCTGG
GGGGACCTTCGGTTTTTCTTTTTCCACCTAAACCGAAAGACACCCTGATGATCAGCAGGACACCAGAAGTGAC
ATGTGTCGTCGTGGACGTATCCCATGAGGACCCGGAGGTGAAGTTCAACTGGTATGTTGATGGTGTGGAAGTG
CATAATGCAAAAACTAAGCCACGGGAGGAACAGTACAATAGCACATACAGAGTAGTTAGCGTTCTGACTGTCC
TGCACCAAGATTGGCTGAACGGGAAGGAATACAAGTGTAAGGTGAGCAACAAAGCCCTGCCCGCTCCAATTGA
GAAAACAATTTCTAAGGCCAAAGGACAGCCCCGGGAGCCACAGGTGTATACTCTGCCGCCTAGCAGGGAGGAA
ATGACCAAGAACCAGGTGAGCCTGACCTGTCTGGTGAAAGGCTTCTACCCCAGCGATATCGCAGTTGAGTGGG
AGAGCAATGGCCAACCCGAGAACAACTACAAGACTACGCCCCCCGTGCTTGATAGCGACGGATCATTTTTCCT
GTACTCAAAACTGACCGTGGACAAAAGCAGATGGCAGCAGGGAAACGTTTTCAGTTGCTCAGTGATGCACGAA
GCGCTGCACAATCATTATACTCAGAAAAGCCTGAGCTTGAGCCCGGGAAAA
SEQ ID NO: 86
CC40.8 L chain amino acid sequence
SYELTQPPSVSVSPGQTARITCSGDALPKRYAYWYQQKSGQAPILVIYEDKKRPSGIPERLSGSKSGTVATLT
ISGAQVEDEADYYCYSTDSSGNHAVFGGGTQLTVLGQPKAAPSVFIFPPSDEQLKSGTASVVCLLNNFYPREA
KVQWKVDNALQSGNSQESVTEQDSKDSTYSLSSTLTLSKADYEKHKVYACEVTHQGLSSPVTKSENRGEC
SEQ ID NO: 87 CV3-25 L chain amino acid sequence
EIVLTQSPSSVSASVGDRVTITCRASQGISSWLAWYQQKPGKAPKLLIYAASSLQSGVPSRFSGSGSGTDFTL
TISSLQPEDFATYYCQQGNSFPYTFGQGTNLEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFNRGEC
SEQ ID NO: 88 CC40.8 L chain nucleotide sequence
AGCTATGAACTGACACAGCCTCCAAGCGTGTCCGTGTCTCCTGGACAGACCGCCAGAATCACATGTAGCGGAG
ATGCCCTGCCTAAGAGATACGCCTACTGGTATCAGCAGAAGTCCGGACAGGCCCCTATCCTGGTCATCTACGA
GGACAAGAAGCGGCCCTCTGGCATCCCTGAGAGACTGAGCGGAAGCAAGTCTGGCACAGTGGCCACACTGACA
ATCTCTGGCGCCCAGGTGGAAGATGAGGCCGACTACTACTGCTACAGCACCGACAGCTCTGGCAACCACGCTG
TGTTTGGCGGAGGTACCCAACTGACCGTGCTGGGCCAACCTAAAGCCGCTCCTAGCGTGTTCATCTTTCCACC
TAGCGACGAGCAGCTGAAGTCTGGCACAGCCTCTGTCGTGTGCCTGCTGAACAACTTCTACCCCAGAGAAGCC
AAGGTGCAGTGGAAGGTGGACAACGCCCTGCAGAGCGGCAATAGCCAAGAGAGCGTGACCGAGCAGGACAGCA
AGGACTCTACCTACAGCCTGAGCAGCACCCTGACACTGAGCAAGGCCGACTACGAGAAGCACAAAGTGTACGC
CTGCGAAGTGACCCACCAGGGCCTTTCTAGCCCTGTGACCAAGAGCTTCAACCGGGGCGAGTGC
SEQ ID NO: 89 CV3-25 L chain nucleotide sequence
GAAATTGTGCTGACACAGAGCCCCAGCAGCGTGTCAGCCTCTGTGGGAGACAGAGTGACCATCACCTGTAGAG
CCAGCCAGGGCATCTCTTCTTGGCTGGCCTGGTATCAGCAGAAGCCTGGCAAGGCCCCTAAGCTGCTGATCTA
TGCCGCTAGCTCTCTGCAGTCTGGCGTGCCCAGCAGATTTTCTGGCAGCGGCTCTGGCACCGACTTCACCCTG
ACCATATCTAGCCTGCAGCCTGAGGACTTCGCCACCTACTATTGCCAGCAGGGCAACAGCTTCCCCTACACCT
TTGGCCAGGGTACCAACCTGGAAATCAAGCGGACAGTGGCCGCTCCTAGCGTGTTCATCTTTCCACCTAGCGA
CGAGCAGCTGAAGTCTGGCACAGCCTCTGTCGTGTGCCTGCTGAACAACTTCTACCCCAGAGAAGCCAAGGTG
CAGTGGAAGGTGGACAACGCCCTGCAGAGCGGCAATAGCCAAGAGAGCGTGACCGAGCAGGACAGCAAGGACT
CTACCTACAGCCTGAGCAGCACCCTGACACTGAGCAAGGCCGACTACGAGAAGCACAAAGTGTACGCCTGCGA
AGTGACCCACCAGGGCCTTTCTAGCCCTGTGACCAAGAGCTTCAACCGGGGCGAGTGC
SEQ ID NO: 90
S2P6 IgG1 H429F H chain amino acid sequence
EVQLVQSGAEVKKPGASVKVSCKASGYTFTSQYMHWVRQAPGQGLEWIGIINPSGVHTSYAQKFQGRVTLTRD
TSTSTLYMELSSLRSEDTAVYYCARGSPKGAFDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSHEDPEVKENWYVDGVEVHNAKTKP
REEQYNSTYRVVSVLTVLHQDWLNGKEYKCKVSNKALPAPIEKTISKAKGQPREPQVYTLPPSRDELTKNQVS
LTCLVKGFYPSDIAVEWESNGQPENNYKTTPPVLDSDGSFFLYSKLTVDKSRWQQGNVFSCSVMFEALHNHYT
QKSLSLSPGK
SEQ ID NO: 91
S2P6 IgG1 H429F H chain nucleotide sequence
GAAGTTCAGCTGGTTCAGTCTGGCGCCGAAGTGAAGAAACCTGGCGCCTCTGTGAAGGTGTCCTGCAAGGCCA
GCGGCTACACCTTTACCAGCCAGTACATGCACTGGGTCCGACAGGCTCCAGGACAGGGACTCGAGTGGATCGG
CATCATCAATCCTAGCGGCGTGCACACCAGCTACGCCCAGAAATTCCAGGGCAGAGTGACCCTGACCAGAGAC
ACCAGCACCAGCACACTGTACATGGAACTGAGCAGCCTGCGGAGCGAGGATACCGCCGTGTACTATTGTGCCA
GAGGCTCTCCTAAGGGCGCCTTCGATTATTGGGGCCAGGGCACACTGGTCACCGTGTCCTCTGCTAGCACAAA
AGGCCCTAGCGTCTTTCCACTCGCCCCATCCTCCAAGAGCACCTCTGGGGGCACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAGCTTGGG
TACCCAGACCTATATCTGCAATGTCAATCATAAACCCAGTAACACAAAGGTAGACAAGAAAGTCGAACCCAAA
TCTTGCGACAAAACTCACACATGCCCTCCTTGCCCCGCCCCCGAACTCCTGGGGGGACCTTCGGTTTTTCTTT
TTCCACCTAAACCGAAAGACACCCTGATGATCAGCAGGACACCAGAAGTGACATGTGTCGTCGTGGACGTATC
CCATGAGGACCCGGAAGTGAAGTTCAACTGGTATGTTGATGGTGTGGAAGTGCATAATGCAAAAACTAAGCCA
CGGGAGGAACAGTACAATAGCACATACAGAGTAGTTAGCGTTCTGACTGTCCTGCACCAAGATTGGCTGAACG
GGAAGGAATACAAGTGTAAGGTGAGCAACAAAGCCCTGCCCGCTCCAATTGAGAAAACAATTTCTAAGGCCAA
AGGACAGCCCCGGGAGCCACAGGTGTATACTCTGCCGCCTAGCAGGGATGAACTGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCGTGCTTGATAGCGACGGATCATTTTTCCTGTACTCAAAACTGACCGTGGA
CAAAAGCAGATGGCAGCAGGGAAACGTTTTCAGTTGCTCAGTGATGTTCGAAGCGCTGCACAATCATTATACT
CAGAAAAGCCTGAGCTTGAGCCCGGGAAAA
SEQ ID NO: 92
CC40. 8 IgG1 H429F H chain amino acid sequence
QVQEVQLLESGGGLVQPGGSLRLSCAASGFTFSSYVMTWARQAPGKGLEWVSAISGTGYTYYADSVKGRFTVS
RDNSKNTLFLQMSSLRAEDTAVYYCAITMAPVVWGQGTTVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSVMFEALHNHYTQ
KSLSLSPGK
SEQ ID NO: 93
CC40.8 IgG1 H429F H chain nucleotide sequence
CAAGTGCAGGAAGTGCAGCTGCTGGAATCTGGCGGAGGACTGGTTCAACCAGGCGGCTCTCTGAGACTGTCTT
GTGCCGCCAGCGGCTTCACCTTCAGCAGCTACGTTATGACCTGGGCCAGACAGGCCCCTGGCAAAGGACTTGA
ATGGGTGTCCGCCATCAGCGGCACAGGCTACACCTACTACGCCGACTCTGTGAAGGGCAGATTCACCGTGTCC
AGAGACAACAGCAAGAATACCCTGTTCCTGCAGATGAGCAGCCTGAGAGCCGAGGATACCGCCGTGTACTACT
GCGCCATTACAATGGCCCCTGTCGTGTGGGGCCAGGGCACAACAGTGACAGTCAGCAGCGCTAGCACAAAAGG
CCCTAGCGTCTTTCCACTCGCCCCATCCTCCAAGAGCACCTCTGGGGGCACTGCGGCTTTGGGCTGCTTGGTG
AAGGATTACTTCCCAGAGCCTGTCACTGTCAGCTGGAATAGCGGGGCTCTGACCAGTGGAGTGCACACCTTTC
CCGCCGTGTTACAGAGCAGCGGCCTCTATAGCCTGAGCAGTGTGGTAACTGTGCCCTCGAGCAGCTTGGGTAC
CCAGACCTATATCTGCAATGTCAATCATAAACCCAGTAACACAAAGGTAGACAAGAAAGTCGAACCCAAATCT
TGCGACAAAACTCACACATGCCCTCCTTGCCCCGCCCCCGAACTCCTGGGGGGACCTTCGGTTTTTCTTTTTC
CACCTAAACCGAAAGACACCCTGATGATCAGCAGGACACCAGAAGTGACATGTGTCGTCGTGGACGTATCCCA
TGAGGACCCGGAGGTGAAGTTCAACTGGTATGTTGATGGTGTGGAAGTGCATAATGCAAAAACTAAGCCACGG
GAGGAACAGTACAATAGCACATACAGAGTAGTTAGCGTTCTGACTGTCCTGCACCAAGATTGGCTGAACGGGA
AGGAATACAAGTGTAAGGTGAGCAACAAAGCCCTGCCCGCTCCAATTGAGAAAACAATTTCTAAGGCCAAAGG
ACAGCCCCGGGAGCCACAGGTGTATACTCTGCCGCCTAGCAGGGATGAACTGACCAAGAACCAGGTGAGCCTG
ACCTGTCTGGTGAAAGGCTTCTACCCCAGCGATATCGCAGTTGAGTGGGAGAGCAATGGCCAACCCGAGAACA
ACTACAAGACTACGCCCCCCGTGCTTGATAGCGACGGATCATTTTTCCTGTACTCAAAACTGACCGTGGACAA
AAGCAGATGGCAGCAGGGAAACGTTTTCAGTTGCTCAGTGATGTTCGAAGCGCTGCACAATCATTATACTCAG
AAAAGCCTGAGCTTGAGCCCGGGAAAA
SEQ ID NO: 94
CV3-25 IgG1 H chain H429F amino acid sequence
QVQLVESGAEVKKPGESLKISCKGSGYTFTRYWIGWVRQMPGKGLEWMGIIYPGDSDTRYSPSFQGHVTISAD
KSISTAYLQWNSLKASDTAMYYCARLPQYCSNGVCQRWFDPWGQGTLVTVSSASTKGPSVFPLAPSSKSTSGG
TAALGCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRWQQGNVFSCSVMFE
ALHNHYTQKSLSLSPGK
SEQ ID NO: 95
CV3-25 IgG1 H chain H429F nucleotide sequence
CAAGTGCAGCTGGTTGAATCTGGCGCCGAAGTGAAGAAGCCTGGCGAGAGCCTGAAGATCAGCTGCAAAGGCA
GCGGCTACACCTTCACCAGATACTGGATCGGCTGGGTCCGACAGATGCCTGGCAAAGGCCTTGAGTGGATGGG
CATCATCTACCCCGGCGACAGCGATACCAGATACAGCCCTAGCTTTCAGGGCCACGTGACCATCAGCGCCGAC
AAGTCTATCAGCACCGCCTACCTGCAGTGGAACAGCCTGAAGGCCAGCGACACCGCCATGTACTACTGTGCCA
GACTGCCCCAGTACTGCAGCAATGGCGTTTGCCAGCGTTGGTTCGATCCTTGGGGACAGGGCACACTGGTCAC
CGTTAGCAGCGCTAGCACAAAAGGCCCTAGCGTCTTTCCACTCGCCCCATCCTCCAAGAGCACCTCTGGGGGC
ACTGCGGCTTTGGGCTGCTTGGTGAAGGATTACTTCCCAGAGCCTGTCACTGTCAGCTGGAATAGCGGGGCTC
TGACCAGTGGAGTGCACACCTTTCCCGCCGTGTTACAGAGCAGCGGCCTCTATAGCCTGAGCAGTGTGGTAAC
TGTGCCCTCGAGCAGCTTGGGTACCCAGACCTATATCTGCAATGTCAATCATAAACCCAGTAACACAAAGGTA
GACAAGAAAGTCGAACCCAAATCTTGCGACAAAACTCACACATGCCCTCCTTGCCCCGCCCCCGAACTCCTGG
GGGGACCTTCGGTTTTTCTTTTTCCACCTAAACCGAAAGACACCCTGATGATCAGCAGGACACCAGAAGTGAC
ATGTGTCGTCGTGGACGTATCCCATGAGGACCCGGAGGTGAAGTTCAACTGGTATGTTGATGGTGTGGAAGTG
CATAATGCAAAAACTAAGCCACGGGAGGAACAGTACAATAGCACATACAGAGTAGTTAGCGTTCTGACTGTCC
TGCACCAAGATTGGCTGAACGGGAAGGAATACAAGTGTAAGGTGAGCAACAAAGCCCTGCCCGCTCCAATTGA
GAAAACAATTTCTAAGGCCAAAGGACAGCCCCGGGAGCCACAGGTGTATACTCTGCCGCCTAGCAGGGATGAA
CTGACCAAGAACCAGGTGAGCCTGACCTGTCTGGTGAAAGGCTTCTACCCCAGCGATATCGCAGTTGAGTGGG
AGAGCAATGGCCAACCCGAGAACAACTACAAGACTACGCCCCCCGTGCTTGATAGCGACGGATCATTTTTCCT
GTACTCAAAACTGACCGTGGACAAAAGCAGATGGCAGCAGGGAAACGTTTTCAGTTGCTCAGTGATGTTCGAA
GCGCTGCACAATCATTATACTCAGAAAAGCCTGAGCTTGAGCCCGGGAAAA
SEQ ID NO: 104
EflACE2-Ig-like-WT H chain amino acid sequence
ETGSTIEEQAKYFLDKFNHEAEDLFYQSSLASWNYNTNITEENVQNMNNAGDKWSAFLKEQSTTAQMYPLQEI
QNLTVKLQLQALQQNGSSVLSEDKSKRLNTILNTMSTIYSTGKVCNPDNPQECLLLEPGLNEIMANSLDYNER
LWAWESWRSEVGKQLRPLYEEYVVLKNEMARANHYEDYGDYWRGDYEVNGVDGYDYSRGQLIEDVEHTFEEIK
PLYEHLHAYVRAKLMNAYPSYISPIGCLPAHLLGDMWGRFWTNLYSLTVPFGQKPNIDVTDAMVDQAWDAQRI
FKEAEKFFVSVGLPNMTQGFWEYSMLTDPGNVQKAVCHPTAWDLGKGDFRILMCTKVTMDDFLTAHHEMGHIQ
YDMAYAAQPFLLRNGANEGFHEAVGEIMSLSAATPKHLKSIGLLSPDFQEDNETEINFLLKQALTIVGTLPFT
YMLEKWRWMVFKGEIPKDQWMKKWWEMKREIVGVVEPVPHDETYCDPASLFHVSNDYSFIRYYTRTLYQFQFQ
EALCQAAKHEGPLHKCDISNSTEAGQKLFNMLRLGKSEPWTLALENVVGAKNMNVRPLLNYFEPLFTWLKDQN
KNSFVGWSTDWSPYADQSIKVRISLKSALGDRAYEWNDNEMYLFRSSVAYAMRQYFLKVKNQMILFGEEDVRV
ANLKPRISENFFVTAPKNVSDIIPRTEVEKAIRMSRSRINDAFRLNDNSLEFLGIQPTLGPPNQPPVSGGGGS
GGGGSASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPVLDSDGSF
FLYSKLTVDKSRWQQGNVFSCSVMHEALHNHYTQKSLSLSPGK
SEQ ID NO: 105
EflACE2-Ig-like-WT H chain nucleotide sequence
GAAACCGGTTCCACCATTGAGGAACAGGCCAAGTACTTTTTGGACAAGTTTAACCACGAAGCCGAAGACCTGT
TCTATCAAAGTTCACTTGCTTCTTGGAATTATAACACCAATATTACTGAAGAGAATGTCCAAAACATGaACAA
CGCCGGCGACAAGTGGAGCGCCTTCCTGAAAGAGCAGAGCACAACAGCCCAGATGTACCCTCTGCAAGAGATC
CAGAACCTGACCGTGAAGCTCCAGCTGCAGGCCCTCCAGCAGAATGGAAGCTCTGTGCTGAGCGAGGACAAGA
GCAAGCGGCTGAACACCATCCTGAATACCATGAGCACCATCTACAGCACCGGCAAAGTGTGCAACCCCGACAA
TCCCCAAGAGTGCCTGCTGCTGGAACCCGGCCTGAATGAGATCATGGCCAACAGCCTGGACTACAACGAGAGA
CTGTGGGCCTGGGAGTCTTGGAGAAGCGAAGTGGGAAAGCAGCTGCGGCCCCTGTACGAGGAATACGTGGTGC
TGAAGAACGAGATGGCCAGAGCCAACCACTACGAGGACTACGGCGACTATTGGAGAGGCGACTACGAAGTGAA
TGGCGTGGACGGCTACGACTACAGCAGAGGCCAGCTGATCGAGGACGTGGAACACACCTTCGAGGAAATCAAG
CCTCTGTACGAGCATCTGCACGCCTACGTGCGGGCCAAGCTGATGAATGCTTACCCCAGCTACATCAGCCCCA
TCGGCTGTCTGCCTGCTCATCTGCTGGGAGACATGTGGGGCAGATTCTGGACCAACCTGTACAGCCTGACAGT
GCCCTTCGGCCAGAAACCTAACATCGACGTGACCGACGCTATGGTGGATCAGGCTTGGGATGCCCAGCGGATC
TTCAAAGAGGCCGAGAAGTTCTTCGTGTCCGTGGGCCTGCCTAATATGACCCAAGGCTTCTGGGAGTACTCCA
TGCTGACAGACCCCGGCAACGTGCAGAAAGCCGTGTGTCATCCTACCGCCTGGGATCTCGGCAAGGGCGACTT
CAGAATCCTGATGTGCACCAAAGTGACGATGGACGACTTCCTGACAGCCCACCACGAGATGGGCCACATCCAG
TACGATATGGCCTACGCCGCTCAGCCCTTCCTGCTGAGAAATGGCGCCAATGAGGGCTTCCATGAGGCCGTGG
GAGAGATCATGAGCCTGTCTGCCGCCACACCTAAGCACCTGAAGTCTATCGGACTGCTGAGCCCCGACTTCCA
AGAGGACAACGAGACAGAGATCAACTTCCTGCTCAAGCAGGCCCTGACCATCGTGGGCACACTGCCCTTTACC
TACATGCTGGAAAAGTGGCGGTGGATGGTCTTTAAGGGCGAGATCCCCAAGGACCAGTGGATGAAGAAATGGT
GGGAGATGAAGCGCGAGATCGTGGGCGTTGTGGAACCTGTGCCTCACGACGAGACATACTGCGATCCTGCCAG
CCTGTTTCACGTGTCCAACGACTACTCCTTCATCCGGTACTACACCCGGACACTGTACCAGTTCCAGTTTCAA
GAGGCTCTGTGCCAGGCCGCCAAGCACGAAGGACCTCTGCACAAGTGCGACATCAGCAACTCTACAGAGGCCG
GACAGAAACTGTTCAACATGCTGCGGCTGGGCAAGAGCGAGCCTTGGACACTGGCTCTGGAAAATGTCGTGGG
CGCCAAGAACATGAATGTGCGGCCACTGCTGAACTACTTCGAGCCCCTGTTCACCTGGCTGAAGGACCAGAAC
AAGAACAGCTTCGTCGGCTGGTCGACCGATTGGAGCCCTTATGCCGATCAGAGCATCAAAGTGCGGATCAGCC
TGAAGTCTGCCCTGGGCGATAGAGCCTACGAGTGGAACGACAACGAGATGTACCTGTTCCGGTCCAGCGTGGC
ATATGCTATGCGGCAGTACTTCCTGAAAGTGAAGAACCAGATGATCCTGTTCGGCGAAGAGGACGTGCGCGTG
GCCAATCTGAAGCCCAGAATCAGCTTCAACTTCTTCGTGACAGCCCCTAAGAACGTGTCCGATATCATCCCCA
GAACCGAGGTGGAAAAGGCCATCAGAATGAGCAGAAGCCGGATCAACGACGCCTTCCGGCTGAACGACAATAG
CCTGGAATTTCTGGGCATCCAGCCTACACTGGGCCCTCCAAATCAGCCTCCTGTTAGCGGAGGAGGAGGATCT
GGAGGAGGAGGATCCGCTAGCACAAAAGGCCCTAGCGTCTTTCCACTCGCCCCATCCTCCAAGAGCACCTCTG
GGGGCACTGCGGCTTTGGGCTGCTTGGTGAAGGATTACTTCCCAGAGCCTGTCACTGTCAGCTGGAATAGCGG
GGCTCTGACCAGTGGAGTGCACACCTTTCCCGCCGTGTTACAGAGCAGCGGCCTCTATAGCCTGAGCAGTGTG
GTAACTGTGCCCTCGAGCAGCTTGGGTACCCAGACCTATATCTGCAATGTCAATCATAAACCCAGTAACACAA
AGGTAGACAAGAAAGTCGAACCCAAATCTTGCGACAAAACTCACACATGCCCTCCTTGCCCCGCCCCCGAGCT
CCTGGGGGGACCTTCGGTTTTTCTTTTTCCACCTAAACCGAAAGACACCCTGATGATCAGCAGGACACCAGAA
GTGACATGTGTCGTCGTGGACGTATCCCATGAGGACCCGGAGGTGAAGTTCAACTGGTATGTTGATGGTGTGG
AAGTGCATAATGCAAAAACTAAGCCACGGGAGGAACAGTACAATAGCACATACAGAGTAGTTAGCGTTCTGAC
TGTCCTGCACCAAGATTGGCTGAACGGGAAGGAATACAAGTGTAAGGTGAGCAACAAAGCCCTGCCCGCTCCA
ATTGAGAAAACAATTTCTAAGGCCAAAGGACAGCCCCGGGAGCCACAGGTGTATACTCTGCCGCCTAGCAGGG
AGGAAATGACCAAGAACCAGGTGAGCCTGACCTGTCTGGTGAAAGGCTTCTACCCCAGCGATATCGCAGTTGA
GTGGGAGAGCAATGGCCAACCCGAGAACAACTACAAGACTACGCCCCCCGTGCTTGATAGCGACGGATCATTT
TTCCTGTACTCAAAACTGACCGTGGACAAAAGCAGATGGCAGCAGGGAAACGTTTTCAGTTGCTCAGTGATGC
ACGAAGCGCTGCACAATCATTATACTCAGAAAAGCCTGAGCTTGAGCCCGGGAAAA
SEQ ID NO: 106
EflACE2 light chain amino acid sequence
ETGSTIEEQAKYFLDKFNHEAEDLFYQSSLASWNYNTNITEENVQNMNNAGDKWSAFLKEQSTTAQMYPLQEI
QNLTVKLQLQALQQNGSSVLSEDKSKRLNTILNTMSTIYSTGKVCNPDNPQECLLLEPGLNEIMANSLDYNER
LWAWESWRSEVGKQLRPLYEEYVVLKNEMARANHYEDYGDYWRGDYEVNGVDGYDYSRGQLIEDVEHTFEEIK
PLYEHLHAYVRAKLMNAYPSYISPIGCLPAHLLGDMWGRFWTNLYSLTVPFGQKPNIDVTDAMVDQAWDAQRI
FKEAEKFFVSVGLPNMTQGFWEYSMLTDPGNVQKAVCHPTAWDLGKGDFRILMCTKVTMDDELTAHHEMGHIQ
YDMAYAAQPFLLRNGANEGFHEAVGEIMSLSAATPKHLKSIGLLSPDFQEDNETEINFLLKQALTIVGTLPFT
YMLEKWRWMVFKGEIPKDQWMKKWWEMKREIVGVVEPVPHDETYCDPASLFHVSNDYSFIRYYTRTLYQFQFQ
EALCQAAKHEGPLHKCDISNSTEAGQKLFNMLRLGKSEPWTLALENVVGAKNMNVRPLLNYFEPLFTWLKDQN
KNSFVGWSTDWSPYADQSIKVRISLKSALGDRAYEWNDNEMYLFRSSVAYAMRQYFLKVKNQMILFGEEDVRV
ANLKPRISFNFFVTAPKNVSDIIPRTEVEKAIRMSRSRINDAFRLNDNSLEFLGIQPTLGPPNQPPVSGGGGS
GGGGSRTVAAPSVFIFPPSDEQLKSGTASVVCLLNNFYPREAKVQWKVDNALQSGNSQESVTEQDSKDSTYSL
SSTLTLSKADYEKHKVYACEVTHQGLSSPVTKSENRGEC
SEQ ID NO: 107
EflACE2 light chain nucleotide sequence
GAAACCGGTTCCACCATTGAGGAACAGGCCAAGTACTTTTTGGACAAGTTTAACCACGAAGCCGAAGACCTGT
TCTATCAAAGTTCACTTGCTTCTTGGAATTATAACACCAATATTACTGAAGAGAATGTCCAAAACATGAACAA
CGCCGGCGACAAGTGGAGCGCCTTCCTGAAAGAGCAGAGCACAACAGCCCAGATGTACCCTCTGCAAGAGATC
CAGAACCTGACCGTGAAGCTCCAGCTGCAGGCCCTCCAGCAGAATGGAAGCTCTGTGCTGAGCGAGGACAAGA
GCAAGCGGCTGAACACCATCCTGAATACCATGAGCACCATCTACAGCACCGGCAAAGTGTGCAACCCCGACAA
TCCCCAAGAGTGCCTGCTGCTGGAACCCGGCCTGAATGAGATCATGGCCAACAGCCTGGACTACAACGAGAGA
CTGTGGGCCTGGGAGTCTTGGAGAAGCGAAGTGGGAAAGCAGCTGCGGCCCCTGTACGAGGAATACGTGGTGC
TGAAGAACGAGATGGCCAGAGCCAACCACTACGAGGACTACGGCGACTATTGGAGAGGCGACTACGAAGTGAA
TGGCGTGGACGGCTACGACTACAGCAGAGGCCAGCTGATCGAGGACGTGGAACACACCTTCGAGGAAATCAAG
CCTCTGTACGAGCATCTGCACGCCTACGTGCGGGCCAAGCTGATGAATGCTTACCCCAGCTACATCAGCCCCA
TCGGCTGTCTGCCTGCTCATCTGCTGGGAGACATGTGGGGCAGATTCTGGACCAACCTGTACAGCCTGACAGT
GCCCTTCGGCCAGAAACCTAACATCGACGTGACCGACGCTATGGTGGATCAGGCTTGGGATGCCCAGCGGATC
TTCAAAGAGGCCGAGAAGTTCTTCGTGTCCGTGGGCCTGCCTAATATGACCCAAGGCTTCTGGGAGTACTCCA
TGCTGACAGACCCCGGCAACGTGCAGAAAGCCGTGTGTCATCCTACCGCCTGGGATCTCGGCAAGGGCGACTT
CAGAATCCTGATGTGCACCAAAGTGACGATGGACGACTTCCTGACAGCCCACCACGAGATGGGCCACATCCAG
TACGATATGGCCTACGCCGCTCAGCCCTTCCTGCTGAGAAATGGCGCCAATGAGGGCTTCCATGAGGCCGTGG
GAGAGATCATGAGCCTGTCTGCCGCCACACCTAAGCACCTGAAGTCTATCGGACTGCTGAGCCCCGACTTCCA
AGAGGACAACGAGACAGAGATCAACTTCCTGCTCAAGCAGGCCCTGACCATCGTGGGCACACTGCCCTTTACC
TACATGCTGGAAAAGTGGCGGTGGATGGTCTTTAAGGGCGAGATCCCCAAGGACCAGTGGATGAAGAAATGGT
GGGAGATGAAGCGCGAGATCGTGGGCGTTGTGGAACCTGTGCCTCACGACGAGACATACTGCGATCCTGCCAG
CCTGTTTCACGTGTCCAACGACTACTCCTTCATCCGGTACTACACCCGGACACTGTACCAGTTCCAGTTTCAA
GAGGCTCTGTGCCAGGCCGCCAAGCACGAAGGACCTCTGCACAAGTGCGACATCAGCAACTCTACAGAGGCCG
GACAGAAACTGTTCAACATGCTGCGGCTGGGCAAGAGCGAGCCTTGGACACTGGCTCTGGAAAATGTCGTGGG
CGCCAAGAACATGAATGTGCGGCCACTGCTGAACTACTTCGAGCCCCTGTTCACCTGGCTGAAGGACCAGAAC
AAGAACAGCTTCGTCGGCTGGTCGACCGATTGGAGCCCTTATGCCGATCAGAGCATCAAAGTGCGGATCAGCC
TGAAGTCTGCCCTGGGCGATAGAGCCTACGAGTGGAACGACAACGAGATGTACCTGTTCCGGTCCAGCGTGGC
ATATGCTATGCGGCAGTACTTCCTGAAAGTGAAGAACCAGATGATCCTGTTCGGCGAAGAGGACGTGCGCGTG
GCCAATCTGAAGCCCAGAATCAGCTTCAACTTCTTCGTGACAGCCCCTAAGAACGTGTCCGATATCATCCCCA
GAACCGAGGTGGAAAAGGCCATCAGAATGAGCAGAAGCCGGATCAACGACGCCTTCCGGCTGAACGACAATAG
CCTGGAATTTCTGGGCATCCAGCCTACACTGGGCCCTCCAAATCAGCCTCCTGTTAGCGGAGGAGGAGGATCT
GGAGGAGGAGGCTCGAGGACAGTGGCCGCTCCTAGCGTGTTCATCTTTCCACCTAGCGACGAGCAGCTGAAGT
CTGGCACAGCCTCTGTCGTGTGCCTGCTGAACAACTTCTACCCCAGAGAAGCCAAGGTGCAGTGGAAGGTGGA
CAACGCCCTGCAGAGCGGCAATAGCCAAGAGAGCGTGACCGAGCAGGACAGCAAGGACTCTACCTACAGCCTG
AGCAGCACCCTGACACTGAGCAAGGCCGACTACGAGAAGCACAAAGTGTACGCCTGCGAAGTGACCCACCAGG
GCCTTTCTAGCCCTGTGACCAAGAGCTTCAACCGGGGCGAGTGC
SEQ ID NO: 108
EflACE2-Ab-like-H429F H chain amino acid sequence
ETGSTIEEQAKYFLDKFNHEAEDLFYQSSLASWNYNTNITEENVQNMNNAGDKWSAFLKEQSTTAQMYPLQEI
QNLTVKLQLQALQQNGSSVLSEDKSKRINTILNTMSTIYSTGKVCNPDNPQECLLLEPGLNEIMANSLDYNER
LWAWESWRSEVGKQLRPLYEEYVVLKNEMARANHYEDYGDYWRGDYEVNGVDGYDYSRGQLIEDVEHTFEEIK
PLYEHLHAYVRAKLMNAYPSYISPIGCLPAHLLGDMWGRFWTNLYSLTVPFGQKPNIDVTDAMVDQAWDAQRI
FKEAEKFFVSVGLPNMTQGFWEYSMLTDPGNVQKAVCHPTAWDLGKGDFRILMCTKVTMDDELTAHHEMGHIQ
YDMAYAAQPFLLRNGANEGFHEAVGEIMSLSAATPKHLKSIGLLSPDFQEDNETEINFLLKQALTIVGTLPFT
YMLEKWRWMVFKGEIPKDQWMKKWWEMKREIVGVVEPVPHDETYCDPASLFHVSNDYSFIRYYTRTLYQFQFQ
EALCQAAKHEGPLHKCDISNSTEAGQKLFNMLRLGKSEPWTLALENVVGAKNMNVRPLLNYFEPLFTWLKDQN
KNSFVGWSTDWSPYADQSIKVRISLKSALGDRAYEWNDNEMYLFRSSVAYAMRQYFLKVKNQMILFGEEDVRV
ANLKPRISENFFVTAPKNVSDIIPRTEVEKAIRMSRSRINDAFRLNDNSLEFLGIQPTLGPPNQPPVSGGGGS
GGGGSASTKGPSVFPLAPSSKSTSGGTAALGCLVKDYFPEPVTVSWNSGALTSGVHTFPAVLQSSGLYSLSSV
VTVPSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSNKALPAP
IEKTISKAKGQPREPQVYTLPPSREEMTKNQVSLTCLVKGFYPSDIAVEWESNGQPENNYKTTPPVLDSDGSF
FLYSKLTVDKSRWQQGNVFSCSVMFEALHNHYTQKSLSLSPGK
SEQ ID NO: 109
EflACE2-Ab-like-H429F H chain nucleotide sequence
GAAACCGGTTCCACCATTGAGGAACAGGCCAAGTACTTTTTGGACAAGTTTAACCACGAAGCCGAAGACCTGT
TCTATCAAAGTTCACTTGCTTCTTGGAATTATAACACCAATATTACTGAAGAGAATGTCCAAAACATGaACAA
CGCCGGCGACAAGTGGAGCGCCTTCCTGAAAGAGCAGAGCACAACAGCCCAGATGTACCCTCTGCAAGAGATC
CAGAACCTGACCGTGAAGCTCCAGCTGCAGGCCCTCCAGCAGAATGGAAGCTCTGTGCTGAGCGAGGACAAGA
GCAAGCGGCTGAACACCATCCTGAATACCATGAGCACCATCTACAGCACCGGCAAAGTGTGCAACCCCGACAA
TCCCCAAGAGTGCCTGCTGCTGGAACCCGGCCTGAATGAGATCATGGCCAACAGCCTGGACTACAACGAGAGA
CTGTGGGCCTGGGAGTCTTGGAGAAGCGAAGTGGGAAAGCAGCTGCGGCCCCTGTACGAGGAATACGTGGTGC
TGAAGAACGAGATGGCCAGAGCCAACCACTACGAGGACTACGGCGACTATTGGAGAGGCGACTACGAAGTGAA
TGGCGTGGACGGCTACGACTACAGCAGAGGCCAGCTGATCGAGGACGTGGAACACACCTTCGAGGAAATCAAG
CCTCTGTACGAGCATCTGCACGCCTACGTGCGGGCCAAGCTGATGAATGCTTACCCCAGCTACATCAGCCCCA
TCGGCTGTCTGCCTGCTCATCTGCTGGGAGACATGTGGGGCAGATTCTGGACCAACCTGTACAGCCTGACAGT
GCCCTTCGGCCAGAAACCTAACATCGACGTGACCGACGCTATGGTGGATCAGGCTTGGGATGCCCAGCGGATC
TTCAAAGAGGCCGAGAAGTTCTTCGTGTCCGTGGGCCTGCCTAATATGACCCAAGGCTTCTGGGAGTACTCCA
TGCTGACAGACCCCGGCAACGTGCAGAAAGCCGTGTGTCATCCTACCGCCTGGGATCTCGGCAAGGGCGACTT
CAGAATCCTGATGTGCACCAAAGTGACGATGGACGACTTCCTGACAGCCCACCACGAGATGGGCCACATCCAG
TACGATATGGCCTACGCCGCTCAGCCCTTCCTGCTGAGAAATGGCGCCAATGAGGGCTTCCATGAGGCCGTGG
GAGAGATCATGAGCCTGTCTGCCGCCACACCTAAGCACCTGAAGTCTATCGGACTGCTGAGCCCCGACTTCCA
AGAGGACAACGAGACAGAGATCAACTTCCTGCTCAAGCAGGCCCTGACCATCGTGGGCACACTGCCCTTTACC
TACATGCTGGAAAAGTGGCGGTGGATGGTCTTTAAGGGCGAGATCCCCAAGGACCAGTGGATGAAGAAATGGT
GGGAGATGAAGCGCGAGATCGTGGGCGTTGTGGAACCTGTGCCTCACGACGAGACATACTGCGATCCTGCCAG
CCTGTTTCACGTGTCCAACGACTACTCCTTCATCCGGTACTACACCCGGACACTGTACCAGTTCCAGTTTCAA
GAGGCTCTGTGCCAGGCCGCCAAGCACGAAGGACCTCTGCACAAGTGCGACATCAGCAACTCTACAGAGGCCG
GACAGAAACTGTTCAACATGCTGCGGCTGGGCAAGAGCGAGCCTTGGACACTGGCTCTGGAAAATGTCGTGGG
CGCCAAGAACATGAATGTGCGGCCACTGCTGAACTACTTCGAGCCCCTGTTCACCTGGCTGAAGGACCAGAAC
AAGAACAGCTTCGTCGGCTGGTCGACCGATTGGAGCCCTTATGCCGATCAGAGCATCAAAGTGCGGATCAGCC
TGAAGTCTGCCCTGGGCGATAGAGCCTACGAGTGGAACGACAACGAGATGTACCTGTTCCGGTCCAGCGTGGC
ATATGCTATGCGGCAGTACTTCCTGAAAGTGAAGAACCAGATGATCCTGTTCGGCGAAGAGGACGTGCGCGTG
GCCAATCTGAAGCCCAGAATCAGCTTCAACTTCTTCGTGACAGCCCCTAAGAACGTGTCCGATATCATCCCCA
GAACCGAGGTGGAAAAGGCCATCAGAATGAGCAGAAGCCGGATCAACGACGCCTTCCGGCTGAACGACAATAG
CCTGGAATTTCTGGGCATCCAGCCTACACTGGGCCCTCCAAATCAGCCTCCTGTTAGCGGAGGAGGAGGATCT
GGAGGAGGAGGATCCGCTAGCACAAAAGGCCCTAGCGTCTTTCCACTCGCCCCATCCTCCAAGAGCACCTCTG
GGGGCACTGCGGCTTTGGGCTGCTTGGTGAAGGATTACTTCCCAGAGCCTGTCACTGTCAGCTGGAATAGCGG
GGCTCTGACCAGTGGAGTGCACACCTTTCCCGCCGTGTTACAGAGCAGCGGCCTCTATAGCCTGAGCAGTGTG
GTAACTGTGCCCTCGAGCAGCTTGGGTACCCAGACCTATATCTGCAATGTCAATCATAAACCCAGTAACACAA
AGGTAGACAAGAAAGTCGAACCCAAATCTTGCGACAAAACTCACACATGCCCTCCTTGCCCCGCCCCCGAGCT
CCTGGGGGGACCTTCGGTTTTTCTTTTTCCACCTAAACCGAAAGACACCCTGATGATCAGCAGGACACCAGAA
GTGACATGTGTCGTCGTGGACGTATCCCATGAGGACCCGGAGGTGAAGTTCAACTGGTATGTTGATGGTGTGG
AAGTGCATAATGCAAAAACTAAGCCACGGGAGGAACAGTACAATAGCACATACAGAGTAGTTAGCGTTCTGAC
TGTCCTGCACCAAGATTGGCTGAACGGGAAGGAATACAAGTGTAAGGTGAGCAACAAAGCCCTGCCCGCTCCA
ATTGAGAAAACAATTTCTAAGGCCAAAGGACAGCCCCGGGAGCCACAGGTGTATACTCTGCCGCCTAGCAGGG
AGGAAATGACCAAGAACCAGGTGAGCCTGACCTGTCTGGTGAAAGGCTTCTACCCCAGCGATATCGCAGTTGA
GTGGGAGAGCAATGGCCAACCCGAGAACAACTACAAGACTACGCCCCCCGTGCTTGATAGCGACGGATCATTT
TTCCTGTACTCAAAACTGACCGTGGACAAAAGCAGATGGCAGCAGGGAAACGTTTTCAGTTGCTCAGTGATGT
TCGAAGCGCTGCACAATCATTATACTCAGAAAAGCCTGAGCTTGAGCCCGGGAAAA
SEQ ID NO: 110
BDR5-1LA/LA/H429F IgG1 H chain amino acid sequence
EVQLQQSGAEVVKPGASVKLSCKASGFNIKDTFIHWVKQAPGQGLEWIGRIDPANTNTKYDPKFQGKATITTD
TSSNTAYMELSSLRSEDTAVYYCVRGLYTYYFDYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKRVEPK
SCDKTHTCPPCPAPEAAGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMFEALHNHYT
QKSLSLSPG
SEQ ID NO: 111
BDR5-1LA/LA/H429F IgG1 H chain nucleotide sequence
GAAGTGCAGCTGCAACAGTCTGGCGCCGAGGTTGTGAAACCTGGCGCCTCTGTGAAGCTGAGCTGTAAAGCCA
GCGGCTTCAACATCAAGGACACCTTCATCCACTGGGTCAAGCAGGCCCCTGGACAGGGACTCGAATGGATCGG
CAGAATCGACCCCGCCAACACCAACACTAAGTACGACCCCAAGTTCCAGGGCAAAGCCACCATCACCACCGAC
ACCAGCAGCAACACCGCCTACATGGAACTGAGCAGCCTGAGAAGCGAGGACACCGCCGTGTACTATTGTGTGC
GGGGCCTGTACACCTACTACTTCGATTATTGGGGCCAGGGTACCCTGGTCACAGTTAGCTCTGCTAGCACAAA
AGGCCCTAGCGTCTTTCCACTCGCCCCATCCTCCAAGAGCACCTCTGGGGGCACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAGCTTGGG
CACCCAGACCTATATCTGCAATGTCAATCATAAACCCAGTAACACAAAGGTAGACAAGAGAGTCGAACCCAAA
TCTTGCGACAAAACTCACACATGCCCTCCTTGCCCCGCCCCCGAAGCCGCGGGGGGACCTTCGGTTTTTCTTT
TTCCACCTAAACCGAAAGACACCCTGATGATCAGCAGGACACCAGAAGTGACATGTGTCGTCGTGGACGTATC
CCATGAGGACCCGGAGGTGAAGTTCAACTGGTATGTTGATGGTGTGGAAGTGCATAATGCAAAAACTAAGCCA
CGGGAGGAACAGTACAATAGCACATACAGAGTAGTTAGCGTTCTGACTGTCCTGCACCAAGATTGGCTGAACG
GGAAGGAATACAAGTGTAAGGTGAGCAACAAAGCCCTGCCCGCTCCAATTGAGAAAACAATTTCTAAGGCCAA
AGGACAGCCCCGGGAGCCACAGGTGTATACTCTGCCGCCTAGCAGGGAGGAAATGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCGTGCTTGATAGCGACGGATCATTTTTCCTGTACTCAAAACTGACCGTGGA
CAAAAGCAGATGGCAGCAGGGAAACGTTTTCAGTTGCTCAGTGATGTTCGAAGCGCTGCACAATCATTATACT
CAGAAAAGCCTGAGCTTGAGCCCGGGA
SEQ ID NO: 112
TDR5-1LA/LA/H429F IgG1 H chain amino acid sequence
QVQLVQSGAEVKKPGASVKVSCKASGFNIKDTHMHWVRQAPGQRLEWIGRIDPANGNTEYDQKFQGRVTITVD
TSASTAYMELSSLRSEDTAVYYCARWGTNVYFAYWGQGTLVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKRVEPK
SCDKTHTCPPCPAPEAAGGPSVFLFPPKPKDTLMISRTPEVTCVVVDVSHEDPEVKENWYVDGVEVHNAKTKP
REEQYNSTYRVVSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCSVMFEALHNHYT
QKSLSLSPG
SEQ ID NO: 113
TDR5-1LA/LA/H429F IgG1 H chain nucleotide sequence
CAGGTTCAGCTGGTTCAGTCTGGCGCCGAAGTGAAGAAACCTGGCGCCTCTGTGAAGGTGTCCTGCAAGGCCA
GCGGCTTCAACATCAAGGACACCCACATGCACTGGGTCCGACAGGCTCCAGGACAGAGACTGGAATGGATCGG
CAGAATCGACCCCGCCAACGGCAACACCGAGTACGACCAGAAATTCCAGGGCAGAGTGACCATCACCGTGGAC
ACATCTGCCAGCACCGCCTACATGGAACTGAGCAGCCTGAGAAGCGAGGACACCGCCGTGTACTACTGTGCCA
GATGGGGCACCAACGTGTACTTCGCCTATTGGGGCCAGGGTACCCTGGTCACAGTTAGCTCTGCTAGCACAAA
AGGCCCTAGCGTCTTTCCACTCGCCCCATCCTCCAAGAGCACCTCTGGGGGCACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAGCTTGGG
CACCCAGACCTATATCTGCAATGTCAATCATAAACCCAGTAACACAAAGGTAGACAAGAGAGTCGAACCCAAA
TCTTGCGACAAAACTCACACATGCCCTCCTTGCCCCGCCCCCGAAGCCGCGGGGGGACCTTCGGTTTTTCTTT
TTCCACCTAAACCGAAAGACACCCTGATGATCAGCAGGACACCAGAAGTGACATGTGTCGTCGTGGACGTATC
CCATGAGGACCCGGAGGTGAAGTTCAACTGGTATGTTGATGGTGTGGAAGTGCATAATGCAAAAACTAAGCCA
CGGGAGGAACAGTACAATAGCACATACAGAGTAGTTAGCGTTCTGACTGTCCTGCACCAAGATTGGCTGAACG
GGAAGGAATACAAGTGTAAGGTGAGCAACAAAGCCCTGCCCGCTCCAATTGAGAAAACAATTTCTAAGGCCAA
AGGACAGCCCCGGGAGCCACAGGTGTATACTCTGCCGCCTAGCAGGGAGGAAATGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCGTGCTTGATAGCGACGGATCATTTTTCCTGTACTCAAAACTGACCGTGGA
CAAAAGCAGATGGCAGCAGGGAAACGTTTTCAGTTGCTCAGTGATGTTCGAAGCGCTGCACAATCATTATACT
CAGAAAAGCCTGAGCTTGAGCCCGGGA
SEQ ID NO: 114
TNP-IgG4-WT (amino acid sequence)
EVQLQESGPSLVKPSQTLSLTCSVTGDSITSGYWNWIRQVPGNKLEYMGFINYSGNTYYNPSLRSRISITRDT
SKNQYFLHLNSVTTEDTATYYCARANWDVFAYWGQGTLVTVSAASTKGPSVFPLAPCSRSTSESTAALGCLVK
DYFPEPVTVSWNSGALTSGVHTFPAVLQSSGLYSLSSVVTVPSSSLGTKTYTCNVDHKPSNTKVDKRVESKYG
PPCPSCPAPEFLGGPSVFLFPPKPKDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQEGNVFSCSVMHEALHNHYTQKSLS
LSLGK
SEQ ID NO: 115
TNP-IgG4-WT (nucleotide sequence)
GAGGTGCAGTTGCAGGAATCTGGTCCCAGTCTAGTTAAGCCCAGCCAGACACTGAGCCTGACATGTAGTGTGA
CCGGTGACAGCATTACAAGCGGCTACTGGAACTGGATCAGACAGGTGCCAGGAAACAAACTCGAATACATGGG
GTTCATCAATTACAGTGGCAATACTTACTATAATCCCAGCCTGAGAAGCAGAATTTCTATAACCAGAGACACC
AGCAAAAACCAGTACTTTCTGCACCTGAACAGCGTAACAACGGAAGACACCGCCACCTATTACTGCGCCAGGG
CCAACTGGGATGTGTTCGCATACTGGGGCCAGGGCACTCTGGTGACGGTGAGCGCCGCGTCGACAAAAGGTCC
CAGCGTGTTTCCCCTGGCTCCTTGTAGCAGAAGCACGTCAGAAAGTACAGCTGCCCTGGGATGCCTGGTGAAA
GATTATTTTCCCGAGCCCGTTACCGTTTCCTGGAATAGTGGCGCCCTGACCTCGGGCGTGCATACTTTTCCCG
CTGTTCTGCAGAGCAGCGGCCTGTATAGCCTGAGCAGTGTGGTGACTGTTCCGTCTAGCAGCCTGGGTACCAA
GACTTATACATGCAACGTGGACCATAAGCCTAGCAATACTAAGGTAGATAAGCGAGTGGAGAGCAAGTACGGC
CCCCCCTGCCCTAGCTGTCCCGCACCTGAGTTTCTAGGCGGGCCCAGCGTGTTTCTGTTCCCCCCGAAGCCTA
AAGATACCCTGATGATCAGCAGAACACCCGAAGTGACCTGCGTGGTTGTGGACGTCAGCCAGGAAGATCCTGA
GGTGCAGTTCAATTGGTACGTGGACGGCGTGGAGGTGCACAATGCTAAGACAAAGCCCCGCGAAGAGCAATTC
AACAGCACATATAGAGTAGTATCTGTGCTGACCGTGCTGCACCAGGATTGGCTGAACGGCAAGGAATACAAAT
GTAAAGTGAGCAACAAGGGCTTACCCAGTAGCATCGAGAAGACCATTAGCAAAGCAAAAGGTCAACCCCGGGA
GCCCCAGGTGTACACCCTGCCTCCGAGCCAAGAGGAAATGACAAAGAATCAGGTGAGCCTGACCTGCCTGGTG
AAGGGATTTTACCCCAGCGATATTGCTGTGGAGTGGGAGAGCAATGGGCAACCAGAGAATAACTACAAAACGA
CCCCACCAGTATTAGACTCGGACGGATCTTTCTTCTTGTATTCGCGGCTGACTGTGGACAAGAGCAGATGGCA
AGAGGGCAATGTTTTCAGCTGTAGCGTTATGCACGAAGCGCTGCACAATCATTATACTCAGAAAAGCCTGAGC
TTGAGCTTAGGAAAA
SEQ ID NO: 116
TNP2x5 EFLG (amino acid sequence)
EVQLQESGPSLVKPSQTLSLTCSVTGDSITSGYWNWIRQFPGNKLEYMGFINYSGNTYYNPSLRSRISITRDT
SKNQYFLHLNSVTTEDTATYYCARANWDVFAYWGQGTLVTVSAASTKGPSVFPLAPCSRSTSESTAALGCLVK
DYFPEPVTVSWNSGALTSGVHTFPAVLQSSGLYSLSSVVTVPSSNFGTQTYTCNVDHKPSNTKVDKTVERKCC
VECPPCPAPEFLGGPSVFLFPPKPKDTLMISRTPEVTCVVVDVSHEDPEVQFNWYVDGVEVHNAKTKPREEQF
NSTFRVVSVLTVVHQDWLNGKEYKCKVSNKGLPAPIEKTISKTKGQPREPQVYTLPPSREEMTKNQVSLTCLV
KGFYPSDIAVEWESNGQPENNYKTTPPMLDSDGSFFLYSKLTVDKSRWQQGNVFSCSVMHEALHNHYTQKSLS
LSPGK
SEQ ID NO: 117
TNP2x5 EFLG (nucleotide sequence)
GAGGTGCAGCTTCAGGAGTCAGGACCTAGCCTCGTGAAACCTTCTCAGACTCTGTCCCTCACATGTTCTGTCA
CTGGCGACTCCATCACCAGTGGTTACTGGAACTGGATCCGGCAATTCCCAGGGAATAAACTTGAGTACATGGG
TTTCATAAATTACAGTGGTAACACTTACTACAATCCATCTCTCAGAAGTCGAATCTCCATCACTCGAGACACA
TCCAAGAACCAGTACTTCCTGCACTTGAATTCTGTGACTACTGAGGACACAGCCACATATTACTGTGCAAGGG
CTAACTGGGACGTCTTTGCTTACTGGGGCCAAGGGACTCTGGTCACTGTCTCTGCAGCCTCCACCAAGGGCCC
ATCGGTCTTCCCCCTGGCGCCCTGCTCCAGGAGCACCTCCGAGAGCACAGCCGCCCTGGGCTGCCTGGTCAAG
GACTACTTCCCCGAACCGGTGACGGTGTCGTGGAACTCAGGCGCTCTGACCAGCGGCGTGCACACCTTCCCAG
CTGTCCTACAGTCCTCAGGACTCTACTCCCTCAGCAGCGTGGTGACCGTGCCCTCCAGCAACTTCGGCACCCA
GACCTACACCTGCAACGTAGATCACAAGCCCAGCAACACCAAGGTGGACAAGACAGTTGAGCGCAAATGTTGT
GTCGAGTGCCCACCGTGCCCAGCACCAgagTttttgggaggaCCGTCAGTCTTCCTCTTCCCCCCAAAACCCA
AGGACACCCTCATGATCTCCCGGACCCCTGAGGTCACGTGCGTGGTGGTGGACGTGAGCCACGAAGACCCCGA
GGTCCAGTTCAACTGGTACGTGGACGGCGTGGAGGTGCATAATGCCAAGACAAAGCCACGGGAGGAGCAGTTC
AACAGCACGTTCCGTGTGGTCAGCGTCCTCACCGTTGTGCACCAGGACTGGCTGAACGGCAAGGAGTACAAGT
GCAAGGTCTCCAACAAAGGCCTCCCAGCCCCCATCGAGAAAACCATCTCCAAAACCAAAGGGCAGCCCCGAGA
ACCACAGGTGTACACCCTGCCCCCATCCCGGGAGGAGATGACCAAGAACCAGGTCAGCCTGACCTGCCTGGTC
AAAGGCTTCTACCCCAGCGACATCGCCGTGGAGTGGGAGAGCAATGGGCAGCCGGAGAACAACTACAAGACCA
CACCTCCCATGCTGGACTCCGACGGCTCCTTCTTCCTCTACAGCAAGCTCACCGTGGACAAGAGCAGGTGGCA
GCAGGGGAACGTCTTCTCATGCTCCGTGATGCATGAGGCTCTGCACAACCACTACACGCAGAAGAGCCTCTCC
CTGTCTCCGGGTAAA
SEQ ID NO: 118
TNP2x4 ELLG (amino acid sequence)
EVQLQESGPSLVKPSQTLSLTCSVTGDSITSGYWNWIRQFPGNKLEYMGFINYSGNTYYNPSLRSRISITRDT
SKNQYFLHLNSVTTEDTATYYCARANWDVFAYWGQGTLVTVSAASTKGPSVFPLAPCSRSTSESTAALGCLVK
DYFPEPVTVSWNSGALTSGVHTFPAVLQSSGLYSLSSVVTVPSSNFGTQTYTCNVDHKPSNTKVDKTVERKCC
VECPPCPAPELLGGPSVFLFPPKPKDTLMISRTPEVTCVVVDVSHEDPEVQFNWYVDGVEVHNAKTKPREEQF
NSTFRVVSVLTVVHQDWLNGKEYKCKVSNKGLPAPIEKTISKTKGQPREPQVYTLPPSREEMTKNQVSLTCLV
KGFYPSDIAVEWESNGQPENNYKTTPPMLDSDGSFFLYSKLTVDKSRWQQGNVFSCSVMHEALHNHYTQKSLS
LSPGK
SEQ ID NO: 119
TNP2x4 ELLG (nucleotide sequence)
GAGGTGCAGCTTCAGGAGTCAGGACCTAGCCTCGTGAAACCTTCTCAGACTCTGTCCCTCACATGTTCTGTCA
CTGGCGACTCCATCACCAGTGGTTACTGGAACTGGATCCGGCAATTCCCAGGGAATAAACTTGAGTACATGGG
TTTCATAAATTACAGTGGTAACACTTACTACAATCCATCTCTCAGAAGTCGAATCTCCATCACTCGAGACACA
TCCAAGAACCAGTACTTCCTGCACTTGAATTCTGTGACTACTGAGGACACAGCCACATATTACTGTGCAAGGG
CTAACTGGGACGTCTTTGCTTACTGGGGCCAAGGGACTCTGGTCACTGTCTCTGCAGCCTCCACCAAGGGCCC
ATCGGTCTTCCCCCTGGCGCCCTGCTCCAGGAGCACCTCCGAGAGCACAGCCGCCCTGGGCTGCCTGGTCAAG
GACTACTTCCCCGAACCGGTGACGGTGTCGTGGAACTCAGGCGCTCTGACCAGCGGCGTGCACACCTTCCCAG
CTGTCCTACAGTCCTCAGGACTCTACTCCCTCAGCAGCGTGGTGACCGTGCCCTCCAGCAACTTCGGCACCCA
GACCTACACCTGCAACGTAGATCACAAGCCCAGCAACACCAAGGTGGACAAGACAGTTGAGCGCAAATGTTGT
GTCGAGTGCCCACCGTGCCCAGCACCAgagcttttgggaggaCCGTCAGTCTTCCTCTTCCCCCCAAAACCCA
AGGACACCCTCATGATCTCCCGGACCCCTGAGGTCACGTGCGTGGTGGTGGACGTGAGCCACGAAGACCCCGA
GGTCCAGTTCAACTGGTACGTGGACGGCGTGGAGGTGCATAATGCCAAGACAAAGCCACGGGAGGAGCAGTTC
AACAGCACGTTCCGTGTGGTCAGCGTCCTCACCGTTGTGCACCAGGACTGGCTGAACGGCAAGGAGTACAAGT
GCAAGGTCTCCAACAAAGGCCTCCCAGCCCCCATCGAGAAAACCATCTCCAAAACCAAAGGGCAGCCCCGAGA
ACCACAGGTGTACACCCTGCCCCCATCCCGGGAGGAGATGACCAAGAACCAGGTCAGCCTGACCTGCCTGGTC
AAAGGCTTCTACCCCAGCGACATCGCCGTGGAGTGGGAGAGCAATGGGCAGCCGGAGAACAACTACAAGACCA
CACCTCCCATGCTGGACTCCGACGGCTCCTTCTTCCTCTACAGCAAGCTCACCGTGGACAAGAGCAGGTGGCA
GCAGGGGAACGTCTTCTCATGCTCCGTGATGCATGAGGCTCTGCACAACCACTACACGCAGAAGAGCCTCTCC
CTGTCTCCGGGTAAA
SEQ ID NO: 120
TNP2x15 EFEG (amino acid sequence)
EVQLQESGPSLVKPSQTLSLTCSVTGDSITSGYWNWIRQVPGNKLEYMGFINYSGNTYYNPSLRSRISITRDT
SKNQYFLHLNSVTTEDTATYYCARANWDVFAYWGQGTLVTVSAASTKGPSVFPLAPCSRSTSESTAALGCLVK
DYFPEPVTVSWNSGALTSGVHTFPAVLQSSGLYSLSSVVTVPSSNFGTQTYTCNVDHKPSNTKVDKTVERKCC
VECPPCPAPEFEGGPSVFLFPPKPKDTLMISRTPEVTCVVVDVSHEDPEVQFNWYVDGVEVHNAKTKPREEQF
NSTFRVVSVLTVVHQDWLNGKEYKCKVSNKGLPAPIEKTISKTKGQPREPQVYTLPPSREEMTKNQVSLTCLV
KGFYPSDIAVEWESNGQPENNYKTTPPMLDSDGSFFLYSKLTVDKSRWQQGNVFSCSVMHEALHNHYTQKSLS
LSLGK
SEQ ID NO: 121
TNP2x15 EFEG (nucleotide sequence)
GAGGTGCAGTTGCAGGAATCTGGTCCCAGTCTAGTTAAGCCCAGCCAGACACTGAGCCTGACATGTAGTGTGA
CCGGTGACAGCATTACAAGCGGCTACTGGAACTGGATCAGACAGGTGCCAGGAAACAAACTCGAATACATGGG
GTTCATCAATTACAGTGGCAATACTTACTATAATCCCAGCCTGAGAAGCAGAATTTCTATAACCAGAGACACC
AGCAAAAACCAGTACTTTCTGCACCTGAACAGCGTAACAACGGAAGACACCGCCACCTATTACTGCGCCAGGG
CCAACTGGGATGTGTTCGCATACTGGGGCCAGGGCACTCTGGTGACGGTGAGCGCCGCGTCGACAAAAGGCCC
TAGCGTCTTTCCACTCGCCCCATGTTCAAGAAGCACCAGCGAGTCAACTGCGGCTTTGGGCTGCTTGGTGAAG
GATTACTTCCCAGAGCCTGTCACTGTCAGCTGGAATAGCGGGGCTCTGACCAGTGGAGTGCACACCTTTCCCG
CCGTGTTACAGAGCAGCGGCCTCTATAGCCTGAGCAGTGTGGTAACTGTGCCCTCGAGCAATTTTGGTACCCA
GACCTATACATGCAATGTCGATCATAAACCCAGTAACACAAAGGTAGACAAGACAGTCGAAAGAAAATGCTGT
GTGGAGTGCCCTCCTTGCCCCGCCCCCGAGTTTGAGGGGGGGCCTTCGGTTTTTCTTTTTCCACCTAAACCGA
AAGACACCCTGATGATCAGCAGGACACCAGAAGTGACATGTGTCGTCGTGGACGTATCCCATGAAGACCCGGA
GGTGCAATTCAACTGGTATGTTGATGGTGTGGAAGTGCATAATGCAAAAACTAAGCCACGGGAGGAACAGTTC
AATAGCACATTCAGAGTAGTTAGCGTTCTGACTGTCGTGCACCAAGATTGGCTGAACGGGAAGGAATACAAGT
GTAAGGTGAGCAACAAAGGTCTGCCCGCTCCAATTGAGAAAACAATTTCTAAGACCAAAGGACAGCCCCGGGA
GCCACAGGTGTATACTCTGCCGCCTAGCAGGGAGGAAATGACCAAGAACCAGGTGAGCCTGACCTGTCTGGTG
AAAGGCTTCTACCCCAGCGATATCGCAGTTGAGTGGGAGAGCAATGGCCAACCCGAGAACAACTACAAGACTA
CGCCCCCCATGCTTGATAGCGACGGATCATTTTTCCTGTACTCAAAACTGACCGTGGACAAAAGCAGATGGCA
GCAGGGAAACGTTTTCAGTTGCTCAGTGATGCACGAAGCgCTGCACAATCATTATACTCAGAAAAGCCTGAGC
TTGAGCTTAGGAAAA
SEQ ID NO: 122
TNP2x17 uHl (amino acid sequence)
EVQLQESGPSLVKPSQTLSLTCSVTGDSITSGYWNWIRQVPGNKLEYMGFINYSGNTYYNPSLRSRISITRDT
SKNQYFLHLNSVTTEDTATYYCARANWDVFAYWGQGTLVTVSAASTKGPSVFPLAPCSRSTSESTAALGCLVK
DYFPEPVTVSWNSGALTSGVHTFPAVLQSSGLYSLSSVVTVPSSNFGTQTYTCNVDHKPSNTKVDKTVEPKSC
DKTHTCPPCPAPPVAGPSVFLFPPKPKDTLMISRTPEVTCVVVDVSHEDPEVQFNWYVDGVEVHNAKTKPREE
QFNSTFRVVSVLTVVHQDWLNGKEYKCKVSNKGLPAPIEKTISKTKGQPREPQVYTLPPSREEMTKNQVSLTC
LVKGFYPSDIAVEWESNGQPENNYKTTPPMLDSDGSFFLYSKLTVDKSRWQQGNVFSCSVMHEALHNHYTQKS
LSLSLGK
SEQ ID NO: 123
TNP2x17 uH1 (nucleotide sequence)
GAGGTGCAGTTGCAGGAATCTGGTCCCAGTCTAGTTAAGCCCAGCCAGACACTGAGCCTGACATGTAGTGTGA
CCGGTGACAGCATTACAAGCGGCTACTGGAACTGGATCAGACAGGTGCCAGGAAACAAACTCGAATACATGGG
GTTCATCAATTACAGTGGCAATACTTACTATAATCCCAGCCTGAGAAGCAGAATTTCTATAACCAGAGACACC
AGCAAAAACCAGTACTTTCTGCACCTGAACAGCGTAACAACGGAAGACACCGCCACCTATTACTGCGCCAGGG
CCAACTGGGATGTGTTCGCATACTGGGGCCAGGGCACTCTGGTGACGGTGAGCGCCGCTAGCACAAAAGGGCC
CAGCGTCTTTCCACTCGCCCCCTGTTCAAGGAGCACCAGCGAGTCAACTGCGGCTTTGGGCTGCTTGGTGAAG
GATTACTTCCCAGAGCCTGTCACTGTCAGCTGGAATAGCGGGGCTCTGACCAGTGGAGTGCACACCTTTCCCG
CCGTGTTACAGAGCAGCGGCCTCTATAGCCTGAGCAGTGTGGTAACTGTGCCTAGCAGCAATTTTGGTACCCA
GACCTATACATGCAATGTCGATCATAAACCCAGTAACACAAAGGTCGACAAGACAGTCGAACCCAAGAGCTGC
GACAAGACCCACACCTGCCCTCCTTGCCCTGCCCCCCCAGTGGCCGGGCCTTCGGTTTTTCTTTTTCCACCTA
AACCGAAAGACACCCTGATGATCTCGAGGACACCAGAGGTCACCTGTGTCGTCGTGGACGTCAGTCACGAGGA
TCCGGAGGTGCAATTCAACTGGTATGTTGATGGTGTGGAAGTGCATAATGCAAAAACTAAGCCCCGGGAGGAA
CAGTTCAATAGCACATTCAGAGTAGTTAGCGTTCTGACTGTCGTGCACCAAGATTGGCTGAACGGGAAGGAAT
ACAAGTGTAAGGTGAGCAACAAAGGTCTGCCCGCTCCAATTGAGAAAACAATTTCTAAGACCAAAGGACAGCC
CCGGGAGCCACAGGTGTATACTCTGCCGCCTAGCAGGGAGGAAATGACCAAGAACCAGGTGAGCCTGACCTGT
CTGGTGAAAGGCTTCTACCCCAGCGATATCGCAGTTGAGTGGGAGAGCAATGGCCAACCCGAGAACAACTACA
AGACTACGCCCCCCATGCTTGATAGCGACGGATCATTTTTCCTGTACTCAAAACTGACCGTGGACAAAAGCAG
ATGGCAGCAGGGAAACGTTTTCAGTTGCTCAGTGATGCACGAAGCgCTGCACAATCATTATACTCAGAAAAGC
CTGAGCTTGAGCTTAGGAAAA
SEQ ID NO: 124
TNP1x32 uH2 (amino acid sequence)
EVQLQESGPSLVKPSQTLSLTCSVTGDSITSGYWNWIRQFPGNKLEYMGFINYSGNTYYNPSLRSRISITRDT
SKNQYFLHLNSVTTEDTATYYCARANWDVFAYWGQGTLVTVSSASTKGPSVFPLAPSSRSTSGGTAALGCLVK
DYFPEPVTVSWNSGALTSGVHTFPAVLQSSGLYSLSSVVTVPSSSLGTQTYICNVNHKPSNTKVDKKVERKCC
VECPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEALHNHYTQKSLS
LSPGK
SEQ ID NO: 125
TNP1x32 uH2 (nucleotide sequence)
GAGGTTCAGCTGCAAGAGTCTGGCCCTAGCCTGGTCAAGCCTAGCCAGACACTGAGCCTGACCTGTAGCGTGA
CCGGCGATAGCATCACAAGCGGCTACTGGAACTGGATCAGACAGTTCCCCGGCAACAAACTTGAGTACATGGG
CTTCATCAACTACAGCGGCAACACCTACTACAACCCCAGCCTGCGGAGCCGGATCAGCATCACCAGAGACACC
AGCAAGAACCAGTACTTCCTGCACCTGAACAGCGTGACCACCGAGGATACCGCCACCTACTATTGCGCCAGAG
CCAACTGGGACGTGTTCGCCTATTGGGGCCAGGGAACACTCGTGACCGTTAGCTCCGCTAGCACAAAGGGCCC
TAGCGTCTTTCCACTCGCCCCCTCCTCCAGGTCTACATCTGGTGGAACAGCCGCTCTGGGCTGTCTGGTCAAG
GACTACTTTCCCGAGCCTGTGACCGTGTCCTGGAATAGCGGAGCACTGACAAGCGGCGTGCACACCTTTCCAG
CCGTGCTGCAATCTAGCGGCCTGTACTCTCTGAGCAGCGTGGTCACAGTGCCTAGCTCTAGCCTCGGTACCCA
GACCTACATCTGCAATGTGAATCACAAGCCCAGCAACACCAAGGTCGACAAGAAGGTGGAAAGAAAATGCTGT
GTGGAGTGTCCTCCATGTCCTGCTCCAGAGCTCCTCGGAGGCCCTTCCGTGTTTCTGTTCCCTCCAAAGCCTA
AGGACACCCTGATGATCTCGAGGACCCCTGAGGTCACCTGTGTGGTGGTTGACGTCAGTCACGAGGATCCTGA
AGTGAAGTTCAATTGGTACGTGGACGGCGTGGAAGTGCACAACGCCAAGACAAAACCCCGGGAGGAACAGTAC
AACAGCACCTACAGAGTGGTGTCCGTGCTGACAGTGCTGCACCAGGATTGGCTGAACGGCAAAGAGTACAAGT
GCAAGGTGTCCAACAAAGCTCTGCCCGCTCCTATCGAGAAAACGATATCCAAGGCCAAGGGCCAGCCTCGGGA
ACCCCAAGTGTATACCTTGCCTCCTAGCCGGGACGAGCTGACCAAGAATCAGGTGTCCCTGACCTGCCTCGTG
AAAGGCTTCTACCCCAGCGACATTGCCGTGGAATGGGAGAGCAATGGCCAGCCTGAGAACAACTACAAGACAA
CCCCTCCTGTGCTGGACAGCGACGGCTCATTCTTCCTGTACAGCAAGCTGACCGTGGACAAGTCGCGATGGCA
GCAGGGCAACGTGTTCTCCTGTTCCGTGATGCACGAAGCGCTGCACAACCACTACACCCAGAAGTCCCTGTCT
CTGAGCCCCGGCAAG
SEQ ID NO: 126
TNP1x3 H429F (amino acid sequence)
EVQLQESGPSLVKPSQTLSLTCSVTGDSITSGYWNWIRQFPGNKLEYMGFINYSGNTYYNPSLRSRISITRDT
SKNQYFLHLNSVTTEDTATYYCARANWDVFAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMFEALHNHYTQK
SLSLSPGK
SEQ ID NO: 127
TNP1x3 H429F (nucleotide sequence)
GAGGTGCAGCTTCAGGAGTCAGGACCTAGCCTCGTGAAACCTTCTCAGACTCTGTCCCTCACATGTTCTGTCA
CTGGCGACTCCATCACCAGTGGTTACTGGAACTGGATCCGGCAATTCCCAGGGAATAAACTTGAGTACATGGG
TTTCATAAATTACAGTGGTAACACTTACTACAATCCATCTCTCAGAAGTCGAATCTCCATCACTCGAGACACA
TCCAAGAACCAGTACTTCCTGCACTTGAATTCTGTGACTACTGAGGACACAGCCACATATTACTGTGCAAGGG
CTAACTGGGACGTCTTTGCTTACTGGGGCCAAGGGACTCTGGTCACTGTCTCTGCAGCCTCCACCAAGGGCCC
ATCGGTCTTCCCCCTGGCACCCTCCTCCAAGAGCACCTCTGGGGGCACAGCGGCCCTGGGCTGCCTGGTCAAG
GACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCA
GACCTACATCTGCAACGTGAATCACAAGCCCAGCAACACCAAGGTGGACAAGAAAGTTGAGCCCAAATCTTGT
GACAAAACTCACACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGGCTGAATGGCAAGG
AGTACAAGTGCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTGACC
TGCCTGGTCAAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACCGTGGACAAGAG
CAGGTGGCAGCAGGGGAACGTCTTCTCATGCTCCGTGATGTTTGAGGCTCTGCACAACCACTACACGCAGAAG
AGCCTCTCCCTGTCTCCGGGTAAA
SEQ ID NO: 128
TNP1x5 E430G (amino acid sequence)
EVQLQESGPSLVKPSQTLSLTCSVTGDSITSGYWNWIRQFPGNKLEYMGFINYSGNTYYNPSLRSRISITRDT
SKNQYFLHLNSVTTEDTATYYCARANWDVFAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMHGALHNHYTQK
SLSLSPGK
SEQ ID NO: 129 TNP1x5 E430G (nucleotide sequence)
GAGGTGCAGCTTCAGGAGTCAGGACCTAGCCTCGTGAAACCTTCTCAGACTCTGTCCCTCACATGTTCTGTCA
CTGGCGACTCCATCACCAGTGGTTACTGGAACTGGATCCGGCAATTCCCAGGGAATAAACTTGAGTACATGGG
TTTCATAAATTACAGTGGTAACACTTACTACAATCCATCTCTCAGAAGTCGAATCTCCATCACTCGAGACACA
TCCAAGAACCAGTACTTCCTGCACTTGAATTCTGTGACTACTGAGGACACAGCCACATATTACTGTGCAAGGG
CTAACTGGGACGTCTTTGCTTACTGGGGCCAAGGGACTCTGGTCACTGTCTCTGCAGCCTCCACCAAGGGCCC
ATCGGTCTTCCCCCTGGCACCCTCCTCCAAGAGCACCTCTGGGGGCACAGCGGCCCTGGGCTGCCTGGTCAAG
GACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCA
GACCTACATCTGCAACGTGAATCACAAGCCCAGCAACACCAAGGTGGACAAGAAAGTTGAGCCCAAATCTTGT
GACAAAACTCACACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGGCTGAATGGCAAGG
AGTACAAGTGCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTGACC
TGCCTGGTCAAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACCGTGGACAAGAG
CAGGTGGCAGCAGGGGAACGTCTTCTCATGCTCCGTGATGCATGGGGCTCTGCACAACCACTACACGCAGAAG
AGCCTCTCCCTGTCTCCGGGTAAA
SEQ ID NO:
130
TNP1x1 H4290 (amino acid sequence)
EVQLQESGPSLVKPSQTLSLTCSVTGDSITSGYWNWIRQFPGNKLEYMGFINYSGNTYYNPSLRSRISITRDT
SKNQYFLHLNSVTTEDTATYYCARANWDVFAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMQEALHNHYTQK
SLSLSPGK
SEQ ID NO: 131
TNP1x1 H4290 (nucleotide sequence)
GAGGTGCAGCTTCAGGAGTCAGGACCTAGCCTCGTGAAACCTTCTCAGACTCTGTCCCTCACATGTTCTGTCA
CTGGCGACTCCATCACCAGTGGTTACTGGAACTGGATCCGGCAATTCCCAGGGAATAAACTTGAGTACATGGG
TTTCATAAATTACAGTGGTAACACTTACTACAATCCATCTCTCAGAAGTCGAATCTCCATCACTCGAGACACA
TCCAAGAACCAGTACTTCCTGCACTTGAATTCTGTGACTACTGAGGACACAGCCACATATTACTGTGCAAGGG
CTAACTGGGACGTCTTTGCTTACTGGGGCCAAGGGACTCTGGTCACTGTCTCTGCAGCCTCCACCAAGGGCCC
ATCGGTCTTCCCCCTGGCACCCTCCTCCAAGAGCACCTCTGGGGGCACAGCGGCCCTGGGCTGCCTGGTCAAG
GACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCA
GACCTACATCTGCAACGTGAATCACAAGCCCAGCAACACCAAGGTGGACAAGAAAGTTGAGCCCAAATCTTGT
GACAAAACTCACACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGGCTGAATGGCAAGG
AGTACAAGTGCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTGACC
TGCCTGGTCAAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACCGTGGACAAGAG
CAGGTGGCAGCAGGGGAACGTCTTCTCATGCTCCGTGATGCAGGAGGCTCTGCACAACCACTACACGCAGAAG
AGCCTCTCCCTGTCTCCGGGTAAA
SEQ ID NO: 132
TNP1x2 H429E (amino acid sequence)
EVQLQESGPSLVKPSQTLSLTCSVTGDSITSGYWNWIRQFPGNKLEYMGFINYSGNTYYNPSLRSRISITRDT
SKNQYFLHLNSVTTEDTATYYCARANWDVFAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMEEALHNHYTQK
SLSLSPGK
SEQ ID NO: 133
TNP1x2 H429E (nucleotide sequence)
GAGGTGCAGCTTCAGGAGTCAGGACCTAGCCTCGTGAAACCTTCTCAGACTCTGTCCCTCACATGTTCTGTCA
CTGGCGACTCCATCACCAGTGGTTACTGGAACTGGATCCGGCAATTCCCAGGGAATAAACTTGAGTACATGGG
TTTCATAAATTACAGTGGTAACACTTACTACAATCCATCTCTCAGAAGTCGAATCTCCATCACTCGAGACACA
TCCAAGAACCAGTACTTCCTGCACTTGAATTCTGTGACTACTGAGGACACAGCCACATATTACTGTGCAAGGG
CTAACTGGGACGTCTTTGCTTACTGGGGCCAAGGGACTCTGGTCACTGTCTCTGCAGCCTCCACCAAGGGCCC
ATCGGTCTTCCCCCTGGCACCCTCCTCCAAGAGCACCTCTGGGGGCACAGCGGCCCTGGGCTGCCTGGTCAAG
GACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCA
GACCTACATCTGCAACGTGAATCACAAGCCCAGCAACACCAAGGTGGACAAGAAAGTTGAGCCCAAATCTTGT
GACAAAACTCACACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGGCTGAATGGCAAGG
AGTACAAGTGCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTGACC
TGCCTGGTCAAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACCGTGGACAAGAG
CAGGTGGCAGCAGGGGAACGTCTTCTCATGCTCCGTGATGGAAGAGGCTCTGCACAACCACTACACGCAGAAG
AGCCTCTCCCTGTCTCCGGGTAAA
SEQ ID NO: 134
TNP1x9 H435A (amino acid sequence)
EVQLQESGPSLVKPSQTLSLTCSVTGDSITSGYWNWIRQFPGNKLEYMGFINYSGNTYYNPSLRSRISITRDT
SKNQYFLHLNSVTTEDTATYYCARANWDVFAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMHEALHNAYTQK
SLSLSPGK
SEQ ID NO: 135
TNP1x9 H435A (nucleotide sequence)
GAGGTGCAGCTTCAGGAGTCAGGACCTAGCCTCGTGAAACCTTCTCAGACTCTGTCCCTCACATGTTCTGTCA
CTGGCGACTCCATCACCAGTGGTTACTGGAACTGGATCCGGCAATTCCCAGGGAATAAACTTGAGTACATGGG
TTTCATAAATTACAGTGGTAACACTTACTACAATCCATCTCTCAGAAGTCGAATCTCCATCACTCGAGACACA
TCCAAGAACCAGTACTTCCTGCACTTGAATTCTGTGACTACTGAGGACACAGCCACATATTACTGTGCAAGGG
CTAACTGGGACGTCTTTGCTTACTGGGGCCAAGGGACTCTGGTCACTGTCTCTGCAGCCTCCACCAAGGGCCC
ATCGGTCTTCCCCCTGGCACCCTCCTCCAAGAGCACCTCTGGGGGCACAGCGGCCCTGGGCTGCCTGGTCAAG
GACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCA
GACCTACATCTGCAACGTGAATCACAAGCCCAGCAACACCAAGGTGGACAAGAAAGTTGAGCCCAAATCTTGT
GACAAAACTCACACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGGCTGAATGGCAAGG
AGTACAAGTGCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTGACC
TGCCTGGTCAAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACCGTGGACAAGAG
CAGGTGGCAGCAGGGGAACGTCTTCTCATGCTCCGTGATGCATGAGGCTCTGCACAACGCCTACACGCAGAAG
AGCCTCTCCCTGTCTCCGGGTAAA
SEQ ID NO: 136
TNP1x10 $131C (amino acid sequence)
EVQLQESGPSLVKPSQTLSLTCSVTGDSITSGYWNWIRQFPGNKLEYMGFINYSGNTYYNPSLRSRISITRDT
SKNQYFLHLNSVTTEDTATYYCARANWDVFAYWGQGTLVTVSAASTKGPSVFPLAPCSKSTSGGTAALGCLVK
DYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMHEALHNHYTQK
SLSLSPGK
SEQ ID NO: 137
TNP1x10 $131C (nucleotide sequence)
GAGGTGCAGCTTCAGGAGTCAGGACCTAGCCTCGTGAAACCTTCTCAGACTCTGTCCCTCACATGTTCTGTCA
CTGGCGACTCCATCACCAGTGGTTACTGGAACTGGATCCGGCAATTCCCAGGGAATAAACTTGAGTACATGGG
TTTCATAAATTACAGTGGTAACACTTACTACAATCCATCTCTCAGAAGTCGAATCTCCATCACTCGAGACACA
TCCAAGAACCAGTACTTCCTGCACTTGAATTCTGTGACTACTGAGGACACAGCCACATATTACTGTGCAAGGG
CTAACTGGGACGTCTTTGCTTACTGGGGCCAAGGGACTCTGGTCACTGTCTCTGCAGCCTCCACCAAGGGCCC
ATCGGTCTTCCCCCTGGCACCCTGCTCCAAGAGCACCTCTGGGGGCACAGCGGCCCTGGGCTGCCTGGTCAAG
GACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCA
GACCTACATCTGCAACGTGAATCACAAGCCCAGCAACACCAAGGTGGACAAGAAAGTTGAGCCCAAATCTTGT
GACAAAACTCACACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGGCTGAATGGCAAGG
AGTACAAGTGCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTGACC
TGCCTGGTCAAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACCGTGGACAAGAG
CAGGTGGCAGCAGGGGAACGTCTTCTCATGCTCCGTGATGCATGAGGCTCTGCACAACCACTACACGCAGAAG
AGCCTCTCCCTGTCTCCGGGTAAA
SEQ ID NO: 138
TNP1x14 $131C uH4 (amino acid sequence)
EVQLQESGPSLVKPSQTLSLTCSVTGDSITSGYWNWIRQFPGNKLEYMGFINYSGNTYYNPSLRSRISITRDT
SKNQYFLHLNSVTTEDTATYYCARANWDVFAYWGQGTLVTVSAASTKGPSVFPLAPCSKSTSGGTAALGCLVK
DYFPEPVTVSWNSGALTSGVHTFPAVLQSSGLYSLSSVVTVPSSSLGTQTYICNVNHKPSNTKVDKKVESKYG
PPCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEALHNHYTQKSLS
LSPGK
SEQ ID NO: 139
TNP1x14 $131C uH5 (nucleotide sequence)
GAGGTGCAGCTTCAGGAGTCAGGACCTAGCCTCGTGAAACCTTCTCAGACTCTGTCCCTCACATGTTCTGTCA
CTGGCGACTCCATCACCAGTGGTTACTGGAACTGGATCCGGCAATTCCCAGGGAATAAACTTGAGTACATGGG
TTTCATAAATTACAGTGGTAACACTTACTACAATCCATCTCTCAGAAGTCGAATCTCCATCACTCGAGACACA
TCCAAGAACCAGTACTTCCTGCACTTGAATTCTGTGACTACTGAGGACACAGCCACATATTACTGTGCAAGGG
CTAACTGGGACGTCTTTGCTTACTGGGGCCAAGGGACTCTGGTCACTGTCTCTGCAGCCTCCACCAAGGGCCC
ATCGGTCTTCCCCCTGGCACCCTGCTCCAAGAGCACCTCTGGGGGCACAGCGGCCCTGGGCTGCCTGGTCAAG
GACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCA
GACCTACATCTGCAACGTGAATCACAAGCCCAGCAACACCAAGGTGGACAAGAAAGTTGAGAGCAAGTACGGC
CCCCCCTGCCCACCGTGCCCAGCACCTGAACTCCTGGGGGGACCGTCAGTCTTCCTCTTCCCCCCAAAACCCA
AGGACACCCTCATGATCTCCCGGACCCCTGAGGTCACATGCGTGGTGGTGGACGTGAGCCACGAAGACCCTGA
GGTCAAGTTCAACTGGTACGTGGACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTGGCTGAATGGCAAGGAGTACAAGT
GCAAGGTCTCCAACAAAGCCCTCCCAGCCCCCATCGAGAAAACCATCTCCAAAGCCAAAGGGCAGCCCCGAGA
ACCACAGGTGTACACCCTGCCCCCATCCCGGGATGAGCTGACCAAGAACCAGGTCAGCCTGACCTGCCTGGTC
AAAGGCTTCTATCCCAGCGACATCGCCGTGGAGTGGGAGAGCAATGGGCAGCCGGAGAACAACTACAAGACCA
CGCCTCCCGTGCTGGACTCCGACGGCTCCTTCTTCCTCTACAGCAAGCTCACCGTGGACAAGAGCAGGTGGCA
GCAGGGGAACGTCTTCTCATGCTCCGTGATGCATGAGGCTCTGCACAACCACTACACGCAGAAGAGCCTCTCC
CTGTCTCCGGGTAAA
SEQ ID NO: 140
TNP1x15 QYN (295-297) NST (amino acid sequence)
EVQLQESGPSLVKPSQTLSLTCSVTGDSITSGYWNWIRQFPGNKLEYMGFINYSGNTYYNPSLRSRISITRDT
SKNQYFLHLNSVTTEDTATYYCARANWDVFAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKENWYVDGVEVHNAKTKPRE
ENSTSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMHEALHNHYTQK
SLSLSPGK
SEQ ID NO: 141
TNP1x15 QYN (295-297) NST (nucleotide sequence)
GAGGTGCAGCTTCAGGAGTCAGGACCTAGCCTCGTGAAACCTTCTCAGACTCTGTCCCTCACATGTTCTGTCA
CTGGCGACTCCATCACCAGTGGTTACTGGAACTGGATCCGGCAATTCCCAGGGAATAAACTTGAGTACATGGG
TTTCATAAATTACAGTGGTAACACTTACTACAATCCATCTCTCAGAAGTCGAATCTCCATCACTCGAGACACA
TCCAAGAACCAGTACTTCCTGCACTTGAATTCTGTGACTACTGAGGACACAGCCACATATTACTGTGCAAGGG
CTAACTGGGACGTCTTTGCTTACTGGGGCCAAGGGACTCTGGTCACTGTCTCTGCAGCCTCCACCAAGGGCCC
ATCGGTCTTCCCCCTGGCACCCTCCTCCAAGAGCACCTCTGGGGGCACAGCGGCCCTGGGCTGCCTGGTCAAG
GACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCA
GACCTACATCTGCAACGTGAATCACAAGCCCAGCAACACCAAGGTGGACAAGAAAGTTGAGCCCAAATCTTGT
GACAAAACTCACACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAG
GAGAACAGCACGAGCACGTACCGTGTGGTCAGCGTCCTCACCGTCCTGCACCAGGACTGGCTGAATGGCAAGG
AGTACAAGTGCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTGACC
TGCCTGGTCAAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACCGTGGACAAGAG
CAGGTGGCAGCAGGGGAACGTCTTCTCATGCTCCGTGATGCATGAGGCTCTGCACAACCACTACACGCAGAAG
AGCCTCTCCCTGTCTCCGGGTAAA
SEQ ID NO: 142
TNP1x12 YNS (296-298) NSS (amino acid sequence)
EVQLQESGPSLVKPSQTLSLTCSVTGDSITSGYWNWIRQFPGNKLEYMGFINYSGNTYYNPSLRSRISITRDT
SKNQYFLHLNSVTTEDTATYYCARANWDVFAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKENWYVDGVEVHNAKTKPRE
EQNSSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMHEALHNHYTQK
SLSLSPGK
SEQ ID NO: 143
TNP1x12 YNS (296-298) NSS (nucleotide sequence)
GAGGTGCAGCTTCAGGAGTCAGGACCTAGCCTCGTGAAACCTTCTCAGACTCTGTCCCTCACATGTTCTGTCA
CTGGCGACTCCATCACCAGTGGTTACTGGAACTGGATCCGGCAATTCCCAGGGAATAAACTTGAGTACATGGG
TTTCATAAATTACAGTGGTAACACTTACTACAATCCATCTCTCAGAAGTCGAATCTCCATCACTCGAGACACA
TCCAAGAACCAGTACTTCCTGCACTTGAATTCTGTGACTACTGAGGACACAGCCACATATTACTGTGCAAGGG
CTAACTGGGACGTCTTTGCTTACTGGGGCCAAGGGACTCTGGTCACTGTCTCTGCAGCCTCCACCAAGGGCCC
ATCGGTCTTCCCCCTGGCACCCTCCTCCAAGAGCACCTCTGGGGGCACAGCGGCCCTGGGCTGCCTGGTCAAG
GACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCA
GACCTACATCTGCAACGTGAATCACAAGCCCAGCAACACCAAGGTGGACAAGAAAGTTGAGCCCAAATCTTGT
GACAAAACTCACACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAG
GAGCAGAACAGCAGCACGTACCGTGTGGTCAGCGTCCTCACCGTCCTGCACCAGGACTGGCTGAATGGCAAGG
AGTACAAGTGCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTGACC
TGCCTGGTCAAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACCGTGGACAAGAG
CAGGTGGCAGCAGGGGAACGTCTTCTCATGCTCCGTGATGCATGAGGCTCTGCACAACCACTACACGCAGAAG
AGCCTCTCCCTGTCTCCGGGTAAA
SEQ ID NO: 144
TNP1x16 ED (269, 270) AA (amino acid sequence)
EVQLQESGPSLVKPSQTLSLTCSVTGDSITSGYWNWIRQFPGNKLEYMGFINYSGNTYYNPSLRSRISITRDT
SKNQYFLHLNSVTTEDTATYYCARANWDVFAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHAAPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMHEALHNHYTQK
SLSLSPGK
SEQ ID NO: 145
TNP1X16 ED (269, 270) AA (nucleotide sequence)
GAGGTGCAGCTTCAGGAGTCAGGACCTAGCCTCGTGAAACCTTCTCAGACTCTGTCCCTCACATGTTCTGTCA
CTGGCGACTCCATCACCAGTGGTTACTGGAACTGGATCCGGCAATTCCCAGGGAATAAACTTGAGTACATGGG
TTTCATAAATTACAGTGGTAACACTTACTACAATCCATCTCTCAGAAGTCGAATCTCCATCACTCGAGACACA
TCCAAGAACCAGTACTTCCTGCACTTGAATTCTGTGACTACTGAGGACACAGCCACATATTACTGTGCAAGGG
CTAACTGGGACGTCTTTGCTTACTGGGGCCAAGGGACTCTGGTCACTGTCTCTGCAGCCTCCACCAAGGGCCC
ATCGGTCTTCCCCCTGGCACCCTCCTCCAAGAGCACCTCTGGGGGCACAGCGGCCCTGGGCTGCCTGGTCAAG
GACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCA
GACCTACATCTGCAACGTGAATCACAAGCCCAGCAACACCAAGGTGGACAAGAAAGTTGAGCCCAAATCTTGT
GACAAAACTCACACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGACGTGAGCCACGC
AGCCCCTGAGGTCAAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGGCTGAATGGCAAGG
AGTACAAGTGCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTGACC
TGCCTGGTCAAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACCGTGGACAAGAG
CAGGTGGCAGCAGGGGAACGTCTTCTCATGCTCCGTGATGCATGAGGCTCTGCACAACCACTACACGCAGAAG
AGCCTCTCCCTGTCTCCGGGTAAA
SEQ ID NO: 146
TNP1x22 H429S (amino acid sequence)
EVQLQESGPSLVKPSQTLSLTCSVTGDSITSGYWNWIRQFPGNKLEYMGFINYSGNTYYNPSLRSRISITRDT
SKNQYFLHLNSVTTEDTATYYCARANWDVFAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMSEALHNHYTQK
SLSLSPGK
SEQ ID NO: 147
TNP1x22 H429S (nucleotide sequence)
GAGGTGCAGCTTCAGGAGTCAGGACCTAGCCTCGTGAAACCTTCTCAGACTCTGTCCCTCACATGTTCTGTCA
CTGGCGACTCCATCACCAGTGGTTACTGGAACTGGATCCGGCAATTCCCAGGGAATAAACTTGAGTACATGGG
TTTCATAAATTACAGTGGTAACACTTACTACAATCCATCTCTCAGAAGTCGAATCTCCATCACTCGAGACACA
TCCAAGAACCAGTACTTCCTGCACTTGAATTCTGTGACTACTGAGGACACAGCCACATATTACTGTGCAAGGG
CTAACTGGGACGTCTTTGCTTACTGGGGCCAAGGGACTCTGGTCACTGTCTCTGCAGCCTCCACCAAGGGCCC
ATCGGTCTTCCCCCTGGCACCCTCCTCCAAGAGCACCTCTGGGGGCACAGCGGCCCTGGGCTGCCTGGTCAAG
GACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCA
GACCTACATCTGCAACGTGAATCACAAGCCCAGCAACACCAAGGTGGACAAGAAAGTTGAGCCCAAATCTTGT
GACAAAACTCACACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGGCTGAATGGCAAGG
AGTACAAGTGCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTGACC
TGCCTGGTCAAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACCGTGGACAAGAG
CAGGTGGCAGCAGGGGAACGTCTTCTCATGCTCCGTGATGTCAGAGGCTCTGCACAACCACTACACGCAGAAG
AGCCTCTCCCTGTCTCCGGGTAAA
SEQ ID NO: 148
TNP1x23 L328F (amino acid sequence)
EVQLQESGPSLVKPSQTLSLTCSVTGDSITSGYWNWIRQFPGNKLEYMGFINYSGNTYYNPSLRSRISITRDT
SKNQYFLHLNSVTTEDTATYYCARANWDVFAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKENWYVDGVEVHNAKTKPRE
EQYNSTYRVVSVLTVLHQDWLNGKEYKCKVSNKAFPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMHEALHNHYTQK
SLSLSPGK
SEQ ID NO: 149
TNP1x23 L328F (nucleotide sequence)
GAGGTGCAGCTTCAGGAGTCAGGACCTAGCCTCGTGAAACCTTCTCAGACTCTGTCCCTCACATGTTCTGTCA
CTGGCGACTCCATCACCAGTGGTTACTGGAACTGGATCCGGCAATTCCCAGGGAATAAACTTGAGTACATGGG
TTTCATAAATTACAGTGGTAACACTTACTACAATCCATCTCTCAGAAGTCGAATCTCCATCACTCGAGACACA
TCCAAGAACCAGTACTTCCTGCACTTGAATTCTGTGACTACTGAGGACACAGCCACATATTACTGTGCAAGGG
CTAACTGGGACGTCTTTGCTTACTGGGGCCAAGGGACTCTGGTCACTGTCTCTGCAGCCTCCACCAAGGGCCC
ATCGGTCTTCCCCCTGGCACCCTCCTCCAAGAGCACCTCTGGGGGCACAGCGGCCCTGGGCTGCCTGGTCAAG
GACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCA
GACCTACATCTGCAACGTGAATCACAAGCCCAGCAACACCAAGGTGGACAAGAAAGTTGAGCCCAAATCTTGT
GACAAAACTCACACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGGCTGAATGGCAAGG
AGTACAAGTGCAAGGTCTCCAACAAAGCCTTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTGACC
TGCCTGGTCAAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACCGTGGACAAGAG
CAGGTGGCAGCAGGGGAACGTCTTCTCATGCTCCGTGATGCATGAGGCTCTGCACAACCACTACACGCAGAAG
AGCCTCTCCCTGTCTCCGGGTAAA
SEQ ID NO: 150
TNP1x24 S267E (amino acid sequence)
EVQLQESGPSLVKPSQTLSLTCSVTGDSITSGYWNWIRQFPGNKLEYMGFINYSGNTYYNPSLRSRISITRDT
SKNQYFLHLNSVTTEDTATYYCARANWDVFAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVEHEDPEVKENWYVDGVEVHNAKTKPRE
EQYNSTYRVVSVLTVLHQDWLNGKEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMHEALHNHYTQK
SLSLSPGK
SEQ ID NO: 151
TNP1X24 S267E (nucleotide sequence)
GAGGTGCAGCTTCAGGAGTCAGGACCTAGCCTCGTGAAACCTTCTCAGACTCTGTCCCTCACATGTTCTGTCA
CTGGCGACTCCATCACCAGTGGTTACTGGAACTGGATCCGGCAATTCCCAGGGAATAAACTTGAGTACATGGG
TTTCATAAATTACAGTGGTAACACTTACTACAATCCATCTCTCAGAAGTCGAATCTCCATCACTCGAGACACA
TCCAAGAACCAGTACTTCCTGCACTTGAATTCTGTGACTACTGAGGACACAGCCACATATTACTGTGCAAGGG
CTAACTGGGACGTCTTTGCTTACTGGGGCCAAGGGACTCTGGTCACTGTCTCTGCAGCCTCCACCAAGGGCCC
ATCGGTCTTCCCCCTGGCACCCTCCTCCAAGAGCACCTCTGGGGGCACAGCGGCCCTGGGCTGCCTGGTCAAG
GACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCA
GACTTACATCTGCAACGTGAATCACAAGCCCAGCAACACCAAGGTGGACAAGAAAGTTGAGCCCAAATCTTGT
GACAAAACTCACACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGACGTGGAGCACGA
AGACCCTGAGGTCAAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGGCTGAATGGCAAGG
AGTACAAGTGCAAGGTCTCCAACAAAGCCCTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTGACC
TGCCTGGTCAAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACCGTGGACAAGAG
CAGGTGGCAGCAGGGGAACGTCTTCTCATGCTCCGTGATGCATGAGGCTCTGCACAACCACTACACGCAGAAG
AGCCTCTCCCTGTCTCCGGGTAAA
SEQ ID NO: 152
TNP1x25 S267E, L328F (amino acid sequence)
EVQLQESGPSLVKPSQTLSLTCSVTGDSITSGYWNWIRQFPGNKLEYMGFINYSGNTYYNPSLRSRISITRDT
SKNQYFLHLNSVTTEDTATYYCARANWDVFAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVEHEDPEVKFNWYVDGVEVHNAKTKPRE
EQYNSTYRVVSVLTVLHQDWLNGKEYKCKVSNKAFPAPIEKTISKAKGQPREPQVYTLPPSRDELTKNQVSLT
CLVKGFYPSDIAVEWESNGQPENNYKTTPPVLDSDGSFFLYSKLTVDKSRWQQGNVFSCSVMHEALHNHYTQK
SLSLSPGK
SEQ ID NO: 153
TNP1x25 S267E, L328F (nucleotide sequence)
GAGGTGCAGCTTCAGGAGTCAGGACCTAGCCTCGTGAAACCTTCTCAGACTCTGTCCCTCACATGTTCTGTCA
CTGGCGACTCCATCACCAGTGGTTACTGGAACTGGATCCGGCAATTCCCAGGGAATAAACTTGAGTACATGGG
TTTCATAAATTACAGTGGTAACACTTACTACAATCCATCTCTCAGAAGTCGAATCTCCATCACTCGAGACACA
TCCAAGAACCAGTACTTCCTGCACTTGAATTCTGTGACTACTGAGGACACAGCCACATATTACTGTGCAAGGG
CTAACTGGGACGTCTTTGCTTACTGGGGCCAAGGGACTCTGGTCACTGTCTCTGCAGCCTCCACCAAGGGCCC
ATCGGTCTTCCCCCTGGCACCCTCCTCCAAGAGCACCTCTGGGGGCACAGCGGCCCTGGGCTGCCTGGTCAAG
GACTACTTCCCCGAACCGGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGCCCTCCAGCAGCTTGGGCACCCA
GACCTACATCTGCAACGTGAATCACAAGCCCAGCAACACCAAGGTGGACAAGAAAGTTGAGCCCAAATCTTGT
GACAAAACTCACACATGCCCACCGTGCCCAGCACCTGAACTCCTGGGGGGACCGTCAGTCTTCCTCTTCCCCC
CAAAACCCAAGGACACCCTCATGATCTCCCGGACCCCTGAGGTCACATGCGTGGTGGTGGACGTGGAGCACGA
AGACCCTGAGGTCAAGTTCAACTGGTACGTGGACGGCGTGGAGGTGCATAATGCCAAGACAAAGCCGCGGGAG
GAGCAGTACAACAGCACGTACCGTGTGGTCAGCGTCCTCACCGTCCTGCACCAGGACTGGCTGAATGGCAAGG
AGTACAAGTGCAAGGTCTCCAACAAAGCCTTCCCAGCCCCCATCGAGAAAACCATCTCCAAAGCCAAAGGGCA
GCCCCGAGAACCACAGGTGTACACCCTGCCCCCATCCCGGGATGAGCTGACCAAGAACCAGGTCAGCCTGACC
TGCCTGGTCAAAGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACCGTGGACAAGAG
CAGGTGGCAGCAGGGGAACGTCTTCTCATGCTCCGTGATGCATGAGGCTCTGCACAACCACTACACGCAGAAG
AGCCTCTCCCTGTCTCCGGGTAAA
SEQ ID NO: 154
Rit-IgG2-WT (amino acid sequence)
QVQLQQPGAELVKPGASVKMSCKASGYTFTSYNMHWVKQTPGRGLEWIGAIYPGNGDTSYNQKFKGKATLTAD
KSSSTAYMQLSSLTSEDSAVYYCARSTYYGGDWYFNVWGAGTTVTVSAASTKGPSVFPLAPCSRSTSESTAAL
GCLVKDYFPEPVTVSWNSGALTSGVHTFPAVLQSSGLYSLSSVVTVPSSNFGTQTYTCNVDHKPSNTKVDKTV
ERKCCVECPPCPAPPVAGPSVFLFPPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKGQPREPQVYTLPPSREEMTKNQVSL
TCLVKGFYPSDIAVEWESNGQPENNYKTTPPMLDSDGSFFLYSKLTVDKSRWQQGNVFSCSVMHEALHNHYTQ
KSLSLSPGK
SEQ ID NO: 155
Rit-IgG2-WT (nucleotide sequence)
CAGGTTCAGCTGCAACAGCCTGGCGCCGAGCTTGTGAAACCTGGCGCCTCTGTGAAGATGAGCTGCAAGGCCA
GCGGCTACACCTTCACCAGCTACAACATGCACTGGGTCAAGCAGACCCCTGGCAGAGGCCTGGAATGGATCGG
AGCCATCTATCCCGGCAACGGCGACACCTCCTACAACCAGAAGTTCAAGGGCAAAGCCACACTGACCGCCGAC
AAGAGCAGCAGCACAGCCTACATGCAGCTGAGCAGCCTGACCAGCGAAGATAGCGCCGTGTACTACTGCGCCA
GAAGCACCTATTACGGCGGCGACTGGTACTTCAACGTGTGGGGAGCTGGtACCACCGTGACAGTTTCTGCCGC
TAGCACAAAAGGCCCTAGCGTCTTTCCACTCGCCCCATGTTCAAGAAGCACCAGCGAGTCAACTGCGGCTTTG
GGCTGCTTGGTGAAGGATTACTTCCCAGAGCCTGTCACTGTCAGCTGGAATAGCGGGGCTCTGACCAGTGGAG
TGCACACCTTTCCCGCCGTGTTACAGAGCAGCGGCCTCTATAGCCTGAGCAGTGTGGTAACTGTGCCCTCGAG
CAATTTTGGcACCCAGACCTATACATGCAATGTCGATCATAAACCCAGTAACACAAAGGTAGACAAGACAGTC
GAAAGAAAATGCTGTGTGGAGTGCCCTCCTTGCCCCGCCCCCCCAGTGGCCGGGCCTTCGGTTTTTCTTTTTC
CACCTAAACCGAAAGACACCCTGATGATCAGCAGGACACCAGAAGTGACATGTGTCGTCGTGGACGTATCCCA
TGAAGACCCGGAGGTGCAATTCAACTGGTATGTTGATGGTGTGGAAGTGCATAATGCAAAAACTAAGCCACGG
GAGGAACAGTTCAATAGCACATTCAGAGTAGTTAGCGTTCTGACTGTCGTGCACCAAGATTGGCTGAACGGGA
AGGAATACAAGTGTAAGGTGAGCAACAAAGGTCTGCCCGCTCCAATTGAGAAAACAATTTCTAAGACCAAAGG
ACAGCCCCGGGAGCCACAGGTGTATACTCTGCCGCCTAGCAGGGAGGAAATGACCAAGAACCAGGTGAGCCTG
ACCTGTCTGGTGAAAGGCTTCTACCCCAGCGATATCGCAGTTGAGTGGGAGAGCAATGGCCAACCCGAGAACA
ACTACAAGACTACGCCCCCCATGCTTGATAGCGACGGATCATTTTTCCTGTACTCAAAACTGACCGTGGACAA
AAGCAGATGGCAGCAGGGAAACGTTTTCAGTTGCTCAGTGATGCACGAAGCGCTGCACAATCATTATACTCAG
AAAAGCCTGAGCTTGAGCccAGGAAAA
SEQ ID NO: 156
Rit-IgG2-HF (amino acid sequence)
QVQLQQPGAELVKPGASVKMSCKASGYTFTSYNMHWVKQTPGRGLEWIGAIYPGNGDTSYNQKFKGKATLTAD
KSSSTAYMQLSSLTSEDSAVYYCARSTYYGGDWYFNVWGAGTTVTVSAASTKGPSVFPLAPCSRSTSESTAAL
GCLVKDYFPEPVTVSWNSGALTSGVHTFPAVLQSSGLYSLSSVVTVPSSNFGTQTYTCNVDHKPSNTKVDKTV
ERKCCVECPPCPAPPVAGPSVFLFPPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKGQPREPQVYTLPPSREEMTKNQVSL
TCLVKGFYPSDIAVEWESNGQPENNYKTTPPMLDSDGSFFLYSKLTVDKSRWQQGNVFSCSVMFEALHNHYTQ
KSLSLSPGK
SEQ ID NO: 157
Rit-IgG2-HF (nucleotide sequence)
CAGGTTCAGCTGCAACAGCCTGGCGCCGAGCTTGTGAAACCTGGCGCCTCTGTGAAGATGAGCTGCAAGGCCA
GCGGCTACACCTTCACCAGCTACAACATGCACTGGGTCAAGCAGACCCCTGGCAGAGGCCTGGAATGGATCGG
AGCCATCTATCCCGGCAACGGCGACACCTCCTACAACCAGAAGTTCAAGGGCAAAGCCACACTGACCGCCGAC
AAGAGCAGCAGCACAGCCTACATGCAGCTGAGCAGCCTGACCAGCGAAGATAGCGCCGTGTACTACTGCGCCA
GAAGCACCTATTACGGCGGCGACTGGTACTTCAACGTGTGGGGAGCTGGtACCACCGTGACAGTTTCTGCCGC
TAGCACAAAAGGCCCTAGCGTCTTTCCACTCGCCCCATGTTCAAGAAGCACCAGCGAGTCAACTGCGGCTTTG
GGCTGCTTGGTGAAGGATTACTTCCCAGAGCCTGTCACTGTCAGCTGGAATAGCGGGGCTCTGACCAGTGGAG
TGCACACCTTTCCCGCCGTGTTACAGAGCAGCGGCCTCTATAGCCTGAGCAGTGTGGTAACTGTGCCCTCGAG
CAATTTTGGcACCCAGACCTATACATGCAATGTCGATCATAAACCCAGTAACACAAAGGTAGACAAGACAGTC
GAAAGAAAATGCTGTGTGGAGTGCCCTCCTTGCCCCGCCCCCCCAGTGGCCGGGCCTTCGGTTTTTCTTTTTC
CACCTAAACCGAAAGACACCCTGATGATCAGCAGGACACCAGAAGTGACATGTGTCGTCGTGGACGTATCCCA
TGAAGACCCGGAGGTGCAATTCAACTGGTATGTTGATGGTGTGGAAGTGCATAATGCAAAAACTAAGCCACGG
GAGGAACAGTTCAATAGCACATTCAGAGTAGTTAGCGTTCTGACTGTCGTGCACCAAGATTGGCTGAACGGGA
AGGAATACAAGTGTAAGGTGAGCAACAAAGGTCTGCCCGCTCCAATTGAGAAAACAATTTCTAAGACCAAAGG
ACAGCCCCGGGAGCCACAGGTGTATACTCTGCCGCCTAGCAGGGAGGAAATGACCAAGAACCAGGTGAGCCTG
ACCTGTCTGGTGAAAGGCTTCTACCCCAGCGATATCGCAGTTGAGTGGGAGAGCAATGGCCAACCCGAGAACA
ACTACAAGACTACGCCCCCCATGCTTGATAGCGACGGATCATTTTTCCTGTACTCAAAACTGACCGTGGACAA
AAGCAGATGGCAGCAGGGAAACGTTTTCAGTTGCTCAGTGATGttCGAAGCGCTGCACAATCATTATACTCAG
AAAAGCCTGAGCTTGAGCccAGGAAAA
SEQ ID NO: 158
Rit-IgG2-FLGG (amino acid sequence)
QVQLQQPGAELVKPGASVKMSCKASGYTFTSYNMHWVKQTPGRGLEWIGAIYPGNGDTSYNQKFKGKATLTAD
KSSSTAYMQLSSLTSEDSAVYYCARSTYYGGDWYFNVWGAGTTVTVSAASTKGPSVFPLAPCSRSTSESTAAL
GCLVKDYFPEPVTVSWNSGALTSGVHTFPAVLQSSGLYSLSSVVTVPSSNFGTQTYTCNVDHKPSNTKVDKTV
ERKCCVECPPCPAPEFLGGPSVFLFPPKPKDTLMISRTPEVTCVVVDVSHEDPEVQFNWYVDGVEVHNAKTKP
REEQFNSTFRVVSVLTVVHQDWLNGKEYKCKVSNKGLPAPIEKTISKTKGQPREPQVYTLPPSREEMTKNQVS
LTCLVKGFYPSDIAVEWESNGQPENNYKTTPPMLDSDGSFFLYSKLTVDKSRWQQGNVFSCSVMHEALHNHYT
QKSLSLSPGK
SEQ ID NO: 159
Rit-IgG2-FLGG (nucleotide sequence)
CAGGTTCAGCTGCAACAGCCTGGCGCCGAGCTTGTGAAACCTGGCGCCTCTGTGAAGATGAGCTGCAAGGCCA
GCGGCTACACCTTCACCAGCTACAACATGCACTGGGTCAAGCAGACCCCTGGCAGAGGCCTGGAATGGATCGG
AGCCATCTATCCCGGCAACGGCGACACCTCCTACAACCAGAAGTTCAAGGGCAAAGCCACACTGACCGCCGAC
AAGAGCAGCAGCACAGCCTACATGCAGCTGAGCAGCCTGACCAGCGAAGATAGCGCCGTGTACTACTGCGCCA
GAAGCACCTATTACGGCGGCGACTGGTACTTCAACGTGTGGGGAGCTGGTACCACCGTGACAGTTTCTGCCGC
TAGCACAAAAGGCCCTAGCGTCTTTCCACTCGCCCCATGTTCAAGAAGCACCAGCGAGTCAACTGCGGCTTTG
GGCTGCTTGGTGAAGGATTACTTCCCAGAGCCTGTCACTGTCAGCTGGAATAGCGGGGCTCTGACCAGTGGAG
TGCACACCTTTCCCGCCGTGTTACAGAGCAGCGGCCTCTATAGCCTGAGCAGTGTGGTAACTGTGCCCTCGAG
CAATTTTGGCACCCAGACCTATACATGCAATGTCGATCATAAACCCAGTAACACAAAGGTAGACAAGACAGTC
GAAAGAAAATGCTGTGTGGAGTGCCCTCCTTGCCCCGCTCCGGAGTTCCTGGGCGGGCCTTCGGTTTTTCTTT
TTCCACCTAAACCGAAAGACACCCTGATGATCAGCAGGACACCAGAAGTGACATGTGTCGTCGTGGACGTATC
CCATGAAGACCCGGAGGTGCAATTCAACTGGTATGTTGATGGTGTGGAAGTGCATAATGCAAAAACTAAGCCA
CGGGAGGAACAGTTCAATAGCACATTCAGAGTAGTTAGCGTTCTGACTGTCGTGCACCAAGATTGGCTGAACG
GGAAGGAATACAAGTGTAAGGTGAGCAACAAAGGTCTGCCCGCTCCAATTGAGAAAACAATTTCTAAGACCAA
AGGACAGCCCCGGGAGCCACAGGTGTATACTCTGCCGCCTAGCAGGGAGGAAATGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCATGCTTGATAGCGACGGATCATTTTTCCTGTACTCAAAACTGACCGTGGA
CAAAAGCAGATGGCAGCAGGGAAACGTTTTCAGTTGCTCAGTGATGCACGAAGCGCTGCACAATCATTATACT
CAGAAAAGCCTGAGCTTGAGCCCAGGAAAA
SEQ ID NO: 160
Rit-IgG2-FLGG-HF (amino acid sequence)
QVQLQQPGAELVKPGASVKMSCKASGYTFTSYNMHWVKQTPGRGLEWIGAIYPGNGDTSYNQKFKGKATLTAD
KSSSTAYMQLSSLTSEDSAVYYCARSTYYGGDWYFNVWGAGTTVTVSAASTKGPSVFPLAPCSRSTSESTAAL
GCLVKDYFPEPVTVSWNSGALTSGVHTFPAVLQSSGLYSLSSVVTVPSSNFGTQTYTCNVDHKPSNTKVDKTV
ERKCCVECPPCPAPEFLGGPSVFLFPPKPKDTLMISRTPEVTCVVVDVSHEDPEVQFNWYVDGVEVHNAKTKP
REEQFNSTFRVVSVLTVVHQDWLNGKEYKCKVSNKGLPAPIEKTISKTKGQPREPQVYTLPPSREEMTKNQVS
LTCLVKGFYPSDIAVEWESNGQPENNYKTTPPMLDSDGSFFLYSKLTVDKSRWQQGNVFSCSVMFEALHNHYT
QKSLSLSPGK
SEQ ID NO: 161
Rit-IgG2-FLGG-HF (nucleotide sequence)
CAGGTTCAGCTGCAACAGCCTGGCGCCGAGCTTGTGAAACCTGGCGCCTCTGTGAAGATGAGCTGCAAGGCCA
GCGGCTACACCTTCACCAGCTACAACATGCACTGGGTCAAGCAGACCCCTGGCAGAGGCCTGGAATGGATCGG
AGCCATCTATCCCGGCAACGGCGACACCTCCTACAACCAGAAGTTCAAGGGCAAAGCCACACTGACCGCCGAC
AAGAGCAGCAGCACAGCCTACATGCAGCTGAGCAGCCTGACCAGCGAAGATAGCGCCGTGTACTACTGCGCCA
GAAGCACCTATTACGGCGGCGACTGGTACTTCAACGTGTGGGGAGCTGGTACCACCGTGACAGTTTCTGCCGC
TAGCACAAAAGGCCCTAGCGTCTTTCCACTCGCCCCATGTTCAAGAAGCACCAGCGAGTCAACTGCGGCTTTG
GGCTGCTTGGTGAAGGATTACTTCCCAGAGCCTGTCACTGTCAGCTGGAATAGCGGGGCTCTGACCAGTGGAG
TGCACACCTTTCCCGCCGTGTTACAGAGCAGCGGCCTCTATAGCCTGAGCAGTGTGGTAACTGTGCCCTCGAG
CAATTTTGGCACCCAGACCTATACATGCAATGTCGATCATAAACCCAGTAACACAAAGGTAGACAAGACAGTC
GAAAGAAAATGCTGTGTGGAGTGCCCTCCTTGCCCCGCTCCGGAGTTCCTGGGCGGGCCTTCGGTTTTTCTTT
TTCCACCTAAACCGAAAGACACCCTGATGATCAGCAGGACACCAGAAGTGACATGTGTCGTCGTGGACGTATC
CCATGAAGACCCGGAGGTGCAATTCAACTGGTATGTTGATGGTGTGGAAGTGCATAATGCAAAAACTAAGCCA
CGGGAGGAACAGTTCAATAGCACATTCAGAGTAGTTAGCGTTCTGACTGTCGTGCACCAAGATTGGCTGAACG
GGAAGGAATACAAGTGTAAGGTGAGCAACAAAGGTCTGCCCGCTCCAATTGAGAAAACAATTTCTAAGACCAA
AGGACAGCCCCGGGAGCCACAGGTGTATACTCTGCCGCCTAGCAGGGAGGAAATGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCATGCTTGATAGCGACGGATCATTTTTCCTGTACTCAAAACTGACCGTGGA
CAAAAGCAGATGGCAGCAGGGAAACGTTTTCAGTTGCTCAGTGATGTTCGAAGCGCTGCACAATCATTATACT
CAGAAAAGCCTGAGCTTGAGCCCAGGAAAA
SEQ ID NO: 162
Rit-IgG2-FLGG EG (amino acid sequence)
QVQLQQPGAELVKPGASVKMSCKASGYTFTSYNMHWVKQTPGRGLEWIGAIYPGNGDTSYNQKFKGKATLTAD
KSSSTAYMQLSSLTSEDSAVYYCARSTYYGGDWYFNVWGAGTTVTVSAASTKGPSVFPLAPCSRSTSESTAAL
GCLVKDYFPEPVTVSWNSGALTSGVHTFPAVLQSSGLYSLSSVVTVPSSNFGTQTYTCNVDHKPSNTKVDKTV
ERKCCVECPPCPAPEFLGGPSVFLFPPKPKDTLMISRTPEVTCVVVDVSHEDPEVQFNWYVDGVEVHNAKTKP
REEQFNSTFRVVSVLTVVHQDWLNGKEYKCKVSNKGLPAPIEKTISKTKGQPREPQVYTLPPSREEMTKNQVS
LTCLVKGFYPSDIAVEWESNGQPENNYKTTPPMLDSDGSFFLYSKLTVDKSRWQQGNVFSCSVMHGALHNHYT
QKSLSLSPGK
SEQ ID NO: 163
Rit-IgG2-FLGG EG (nucleotide sequence)
CAGGTTCAGCTGCAACAGCCTGGCGCCGAGCTTGTGAAACCTGGCGCCTCTGTGAAGATGAGCTGCAAGGCCA
GCGGCTACACCTTCACCAGCTACAACATGCACTGGGTCAAGCAGACCCCTGGCAGAGGCCTGGAATGGATCGG
AGCCATCTATCCCGGCAACGGCGACACCTCCTACAACCAGAAGTTCAAGGGCAAAGCCACACTGACCGCCGAC
AAGAGCAGCAGCACAGCCTACATGCAGCTGAGCAGCCTGACCAGCGAAGATAGCGCCGTGTACTACTGCGCCA
GAAGCACCTATTACGGCGGCGACTGGTACTTCAACGTGTGGGGAGCTGGTACCACCGTGACAGTTTCTGCCGC
TAGCACAAAAGGCCCTAGCGTCTTTCCACTCGCCCCATGTTCAAGAAGCACCAGCGAGTCAACTGCGGCTTTG
GGCTGCTTGGTGAAGGATTACTTCCCAGAGCCTGTCACTGTCAGCTGGAATAGCGGGGCTCTGACCAGTGGAG
TGCACACCTTTCCCGCCGTGTTACAGAGCAGCGGCCTCTATAGCCTGAGCAGTGTGGTAACTGTGCCCTCGAG
CAATTTTGGCACCCAGACCTATACATGCAATGTCGATCATAAACCCAGTAACACAAAGGTAGACAAGACAGTC
GAAAGAAAATGCTGTGTGGAGTGCCCTCCTTGCCCCGCTCCGGAGTTCCTGGGCGGGCCTTCGGTTTTTCTTT
TTCCACCTAAACCGAAAGACACCCTGATGATCAGCAGGACACCAGAAGTGACATGTGTCGTCGTGGACGTATC
CCATGAAGACCCGGAGGTGCAATTCAACTGGTATGTTGATGGTGTGGAAGTGCATAATGCAAAAACTAAGCCA
CGGGAGGAACAGTTCAATAGCACATTCAGAGTAGTTAGCGTTCTGACTGTCGTGCACCAAGATTGGCTGAACG
GGAAGGAATACAAGTGTAAGGTGAGCAACAAAGGTCTGCCCGCTCCAATTGAGAAAACAATTTCTAAGACCAA
AGGACAGCCCCGGGAGCCACAGGTGTATACTCTGCCGCCTAGCAGGGAGGAAATGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCATGCTTGATAGCGACGGATCATTTTTCCTGTACTCAAAACTGACCGTGGA
CAAAAGCAGATGGCAGCAGGGAAACGTTTTCAGTTGCTCAGTGATGCACGGAGCGCTGCACAATCATTATACT
CAGAAAAGCCTGAGCTTGAGCCCAGGAAAA
SEQ ID NO: 164
Rit-IgG2-LLGG (amino acid sequence)
QVQLQQPGAELVKPGASVKMSCKASGYTFTSYNMHWVKQTPGRGLEWIGAIYPGNGDTSYNQKFKGKATLTAD
KSSSTAYMQLSSLTSEDSAVYYCARSTYYGGDWYFNVWGAGTTVTVSAASTKGPSVFPLAPCSRSTSESTAAL
GCLVKDYFPEPVTVSWNSGALTSGVHTFPAVLQSSGLYSLSSVVTVPSSNFGTQTYTCNVDHKPSNTKVDKTV
ERKCCVECPPCPAPELLGGPSVFLFPPKPKDTLMISRTPEVTCVVVDVSHEDPEVQFNWYVDGVEVHNAKTKP
REEQFNSTFRVVSVLTVVHQDWLNGKEYKCKVSNKGLPAPIEKTISKTKGQPREPQVYTLPPSREEMTKNQVS
LTCLVKGFYPSDIAVEWESNGQPENNYKTTPPMLDSDGSFFLYSKLTVDKSRWQQGNVFSCSVMHEALHNHYT
QKSLSLSPGK
SEQ ID NO: 165
Rit-IgG2-LLGG (nucleotide sequence)
CAGGTTCAGCTGCAACAGCCTGGCGCCGAGCTTGTGAAACCTGGCGCCTCTGTGAAGATGAGCTGCAAGGCCA
GCGGCTACACCTTCACCAGCTACAACATGCACTGGGTCAAGCAGACCCCTGGCAGAGGCCTGGAATGGATCGG
AGCCATCTATCCCGGCAACGGCGACACCTCCTACAACCAGAAGTTCAAGGGCAAAGCCACACTGACCGCCGAC
AAGAGCAGCAGCACAGCCTACATGCAGCTGAGCAGCCTGACCAGCGAAGATAGCGCCGTGTACTACTGCGCCA
GAAGCACCTATTACGGCGGCGACTGGTACTTCAACGTGTGGGGAGCTGGTACCACCGTGACAGTTTCTGCCGC
TAGCACAAAAGGCCCTAGCGTCTTTCCACTCGCCCCATGTTCAAGAAGCACCAGCGAGTCAACTGCGGCTTTG
GGCTGCTTGGTGAAGGATTACTTCCCAGAGCCTGTCACTGTCAGCTGGAATAGCGGGGCTCTGACCAGTGGAG
TGCACACCTTTCCCGCCGTGTTACAGAGCAGCGGCCTCTATAGCCTGAGCAGTGTGGTAACTGTGCCCTCGAG
CAATTTTGGCACCCAGACCTATACATGCAATGTCGATCATAAACCCAGTAACACAAAGGTAGACAAGACAGTC
GAAAGAAAATGCTGTGTGGAGTGCCCTCCTTGCCCCGCTCCGGAGCTGCTGGGCGGGCCTTCGGTTTTTCTTT
TTCCACCTAAACCGAAAGACACCCTGATGATCAGCAGGACACCAGAAGTGACATGTGTCGTCGTGGACGTATC
CCATGAAGACCCGGAGGTGCAATTCAACTGGTATGTTGATGGTGTGGAAGTGCATAATGCAAAAACTAAGCCA
CGGGAGGAACAGTTCAATAGCACATTCAGAGTAGTTAGCGTTCTGACTGTCGTGCACCAAGATTGGCTGAACG
GGAAGGAATACAAGTGTAAGGTGAGCAACAAAGGTCTGCCCGCTCCAATTGAGAAAACAATTTCTAAGACCAA
AGGACAGCCCCGGGAGCCACAGGTGTATACTCTGCCGCCTAGCAGGGAGGAAATGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCATGCTTGATAGCGACGGATCATTTTTCCTGTACTCAAAACTGACCGTGGA
CAAAAGCAGATGGCAGCAGGGAAACGTTTTCAGTTGCTCAGTGATGCACGAAGCGCTGCACAATCATTATACT
CAGAAAAGCCTGAGCTTGAGCCCAGGAAAA
SEQ ID NO: 166
Rit-IgG2-LLGG HF (amino acid sequence)
QVQLQQPGAELVKPGASVKMSCKASGYTFTSYNMHWVKQTPGRGLEWIGAIYPGNGDTSYNQKFKGKATLTAD
KSSSTAYMQLSSLTSEDSAVYYCARSTYYGGDWYFNVWGAGTTVTVSAASTKGPSVFPLAPCSRSTSESTAAL
GCLVKDYFPEPVTVSWNSGALTSGVHTFPAVLQSSGLYSLSSVVTVPSSNFGTQTYTCNVDHKPSNTKVDKTV
ERKCCVECPPCPAPELLGGPSVFLFPPKPKDTLMISRTPEVTCVVVDVSHEDPEVQFNWYVDGVEVHNAKTKP
REEQFNSTFRVVSVLTVVHQDWLNGKEYKCKVSNKGLPAPIEKTISKTKGQPREPQVYTLPPSREEMTKNQVS
LTCLVKGFYPSDIAVEWESNGQPENNYKTTPPMLDSDGSFFLYSKLTVDKSRWQQGNVFSCSVMFEALHNHYT
QKSLSLSPGK
SEQ ID NO: 167
Rit-IgG2-LLGG HF (nucleotide sequence)
CAGGTTCAGCTGCAACAGCCTGGCGCCGAGCTTGTGAAACCTGGCGCCTCTGTGAAGATGAGCTGCAAGGCCA
GCGGCTACACCTTCACCAGCTACAACATGCACTGGGTCAAGCAGACCCCTGGCAGAGGCCTGGAATGGATCGG
AGCCATCTATCCCGGCAACGGCGACACCTCCTACAACCAGAAGTTCAAGGGCAAAGCCACACTGACCGCCGAC
AAGAGCAGCAGCACAGCCTACATGCAGCTGAGCAGCCTGACCAGCGAAGATAGCGCCGTGTACTACTGCGCCA
GAAGCACCTATTACGGCGGCGACTGGTACTTCAACGTGTGGGGAGCTGGTACCACCGTGACAGTTTCTGCCGC
TAGCACAAAAGGCCCTAGCGTCTTTCCACTCGCCCCATGTTCAAGAAGCACCAGCGAGTCAACTGCGGCTTTG
GGCTGCTTGGTGAAGGATTACTTCCCAGAGCCTGTCACTGTCAGCTGGAATAGCGGGGCTCTGACCAGTGGAG
TGCACACCTTTCCCGCCGTGTTACAGAGCAGCGGCCTCTATAGCCTGAGCAGTGTGGTAACTGTGCCCTCGAG
CAATTTTGGCACCCAGACCTATACATGCAATGTCGATCATAAACCCAGTAACACAAAGGTAGACAAGACAGTC
GAAAGAAAATGCTGTGTGGAGTGCCCTCCTTGCCCCGCTCCGGAGCTGCTGGGCGGGCCTTCGGTTTTTCTTT
TTCCACCTAAACCGAAAGACACCCTGATGATCAGCAGGACACCAGAAGTGACATGTGTCGTCGTGGACGTATC
CCATGAAGACCCGGAGGTGCAATTCAACTGGTATGTTGATGGTGTGGAAGTGCATAATGCAAAAACTAAGCCA
CGGGAGGAACAGTTCAATAGCACATTCAGAGTAGTTAGCGTTCTGACTGTCGTGCACCAAGATTGGCTGAACG
GGAAGGAATACAAGTGTAAGGTGAGCAACAAAGGTCTGCCCGCTCCAATTGAGAAAACAATTTCTAAGACCAA
AGGACAGCCCCGGGAGCCACAGGTGTATACTCTGCCGCCTAGCAGGGAGGAAATGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCATGCTTGATAGCGACGGATCATTTTTCCTGTACTCAAAACTGACCGTGGA
CAAAAGCAGATGGCAGCAGGGAAACGTTTTCAGTTGCTCAGTGATGTTCGAAGCGCTGCACAATCATTATACT
CAGAAAAGCCTGAGCTTGAGCCCAGGAAAATGA
SEQ ID NO: 168
Rit-IgG2-LLGG EG (amino acid sequence)
QVQLQQPGAELVKPGASVKMSCKASGYTFTSYNMHWVKQTPGRGLEWIGAIYPGNGDTSYNQKFKGKATLTAD
KSSSTAYMQLSSLTSEDSAVYYCARSTYYGGDWYFNVWGAGTTVTVSAASTKGPSVFPLAPCSRSTSESTAAL
GCLVKDYFPEPVTVSWNSGALTSGVHTFPAVLQSSGLYSLSSVVTVPSSNFGTQTYTCNVDHKPSNTKVDKTV
ERKCCVECPPCPAPELLGGPSVFLFPPKPKDTLMISRTPEVTCVVVDVSHEDPEVQFNWYVDGVEVHNAKTKP
REEQFNSTFRVVSVLTVVHQDWLNGKEYKCKVSNKGLPAPIEKTISKTKGQPREPQVYTLPPSREEMTKNQVS
LTCLVKGFYPSDIAVEWESNGQPENNYKTTPPMLDSDGSFFLYSKLTVDKSRWQQGNVFSCSVMHGALHNHYT
QKSLSLSPGK
SEQ ID NO: 169
Rit-IgG2-LLGG EG (nucleotide sequence)
CAGGTTCAGCTGCAACAGCCTGGCGCCGAGCTTGTGAAACCTGGCGCCTCTGTGAAGATGAGCTGCAAGGCCA
GCGGCTACACCTTCACCAGCTACAACATGCACTGGGTCAAGCAGACCCCTGGCAGAGGCCTGGAATGGATCGG
AGCCATCTATCCCGGCAACGGCGACACCTCCTACAACCAGAAGTTCAAGGGCAAAGCCACACTGACCGCCGAC
AAGAGCAGCAGCACAGCCTACATGCAGCTGAGCAGCCTGACCAGCGAAGATAGCGCCGTGTACTACTGCGCCA
GAAGCACCTATTACGGCGGCGACTGGTACTTCAACGTGTGGGGAGCTGGTACCACCGTGACAGTTTCTGCCGC
TAGCACAAAAGGCCCTAGCGTCTTTCCACTCGCCCCATGTTCAAGAAGCACCAGCGAGTCAACTGCGGCTTTG
GGCTGCTTGGTGAAGGATTACTTCCCAGAGCCTGTCACTGTCAGCTGGAATAGCGGGGCTCTGACCAGTGGAG
TGCACACCTTTCCCGCCGTGTTACAGAGCAGCGGCCTCTATAGCCTGAGCAGTGTGGTAACTGTGCCCTCGAG
CAATTTTGGCACCCAGACCTATACATGCAATGTCGATCATAAACCCAGTAACACAAAGGTAGACAAGACAGTC
GAAAGAAAATGCTGTGTGGAGTGCCCTCCTTGCCCCGCTCCGGAGCTGCTGGGCGGGCCTTCGGTTTTTCTTT
TTCCACCTAAACCGAAAGACACCCTGATGATCAGCAGGACACCAGAAGTGACATGTGTCGTCGTGGACGTATC
CCATGAAGACCCGGAGGTGCAATTCAACTGGTATGTTGATGGTGTGGAAGTGCATAATGCAAAAACTAAGCCA
CGGGAGGAACAGTTCAATAGCACATTCAGAGTAGTTAGCGTTCTGACTGTCGTGCACCAAGATTGGCTGAACG
GGAAGGAATACAAGTGTAAGGTGAGCAACAAAGGTCTGCCCGCTCCAATTGAGAAAACAATTTCTAAGACCAA
AGGACAGCCCCGGGAGCCACAGGTGTATACTCTGCCGCCTAGCAGGGAGGAAATGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCATGCTTGATAGCGACGGATCATTTTTCCTGTACTCAAAACTGACCGTGGA
CAAAAGCAGATGGCAGCAGGGAAACGTTTTCAGTTGCTCAGTGATGCACGGAGCGCTGCACAATCATTATACT
CAGAAAAGCCTGAGCTTGAGCCCAGGAAAA
SEQ ID NO: 170
Rit-IgG3-KVH WT (amino acid sequence)
QVQLQQPGAELVKPGASVKMSCKASGYTFTSYNMHWVKQTPGRGLEWIGAIYPGNGDTSYNQKFKGKATLTAD
KSSSTAYMQLSSLTSEDSAVYYCARSTYYGGDWYFNVWGAGTTVTVSAASTKGPSVFPLAPCSRSTSGGTAAL
GCLVKDYFPEPVTVSWNSGALTSGVHTFPAVLQSSGLYSLSSVVTVPSSSLGTQTYTCNVNHKPSNTKVDKRV
ELKTPLGDTTHTCPRCPEPKSCDTPPPCPRCPEPKSCDTPPPCPRCPEPKSCDTPPPCPRCPAPELLGGPSVF
LFPPKPKDTLMISRTPEVTCVVVDVSHEDPEVQFKWYVDGVEVHNAKTKLREEQYNSTFRVVSVLTVLHQDWL
NGKEYKCKVSNKALPAPIEKTISKTKGQPREPQVYTLPPSREEMTKNQVSLTCLVKGFYPSDIAVEWESNGQP
ENNYKTTPPVLDSDGSFFLYSKLTVDKSRWQQGNIFSCSVMHEALHNHYTQKSLSLSPGK
SEQ ID NO: 171
Rit-IgG3-KVH WT (nucleotide sequence)
CAGGTTCAGCTGCAACAGCCTGGCGCCGAGCTTGTGAAACCTGGCGCCTCTGTGAAGATGAGCTGCAAGGCCA
GCGGCTACACCTTCACCAGCTACAACATGCACTGGGTCAAGCAGACCCCTGGCAGAGGCCTGGAATGGATCGG
AGCCATCTATCCCGGCAACGGCGACACCTCCTACAACCAGAAGTTCAAGGGCAAAGCCACACTGACCGCCGAC
AAGAGCAGCAGCACAGCCTACATGCAGCTGAGCAGCCTGACCAGCGAAGATAGCGCCGTGTACTACTGCGCCA
GAAGCACCTATTACGGCGGCGACTGGTACTTCAACGTGTGGGGAGCTGGTACCACCGTGACAGTTTCTGCCGC
TAGCACAAAAGGCCCTAGCGTCTTTCCACTCGCCCCATGTTCTAGAAGCACCTCTGGGGGCACTGCGGCTTTG
GGCTGCTTGGTGAAGGATTACTTCCCAGAGCCTGTCACTGTCAGCTGGAATAGCGGGGCTCTGACCAGTGGAG
TGCACACCTTTCCCGCCGTGTTACAGAGCAGCGGCCTCTATAGCCTGAGCAGTGTGGTAACTGTGCCCTCGAG
CAGCTTGGGCACCCAGACCTATACCTGCAATGTCAATCATAAACCCAGTAACACAAAGGTAGACAAGCGGGTC
GAACTGAAAACCCCCCTGGGCGACACCACCCACACCTGTCCTAGATGCCCCGAGCCCAAGTCCTGCGATACCC
CTCCACCTTGCCCCAGGTGCCCTGAGCCTAAGAGCTGTGATACCCCCCCTCCCTGCCCTCGGTGTCCAGAGCC
TAAGTCTTGTGACACCCCACCCCCATGTCCAAGGTGCCCCGCCCCCGAACTCCTGGGGGGACCTTCGGTTTTT
CTTTTTCCACCTAAACCGAAAGACACCCTGATGATCAGCAGGACACCAGAAGTGACATGTGTCGTCGTGGACG
TATCCCATGAGGACCCGGAGGTGCAGTTCAAGTGGTATGTTGATGGTGTGGAAGTGCATAATGCAAAAACTAA
GCTGCGGGAGGAACAGTACAATAGCACATTCAGAGTAGTTAGCGTTCTGACTGTCCTGCACCAAGATTGGCTG
AACGGGAAGGAATACAAGTGTAAGGTGAGCAACAAAGCCCTGCCCGCTCCAATTGAGAAAACAATTTCTAAGA
CCAAAGGACAGCCCCGGGAGCCACAGGTGTATACTCTGCCGCCTAGCAGGGAAGAGATGACCAAGAACCAGGT
GAGCCTGACCTGTCTGGTGAAAGGCTTCTACCCCAGCGATATCGCAGTTGAGTGGGAGAGCAATGGCCAACCC
GAGAACAACTACAAGACTACGCCCCCCGTGCTTGATAGCGACGGATCATTTTTCCTGTACTCAAAACTGACCG
TGGACAAAAGCAGATGGCAGCAGGGAAACATCTTCAGTTGCTCAGTGATGCACGAAGCGCTGCACAATCATTA
TACTCAGAAAAGCCTGAGCTTGAGCCCGGGAAAA
SEQ ID NO: 172
Rit-IgG3-KVH HF (amino acid sequence)
QVQLQQPGAELVKPGASVKMSCKASGYTFTSYNMHWVKQTPGRGLEWIGAIYPGNGDTSYNQKFKGKATLTAD
KSSSTAYMQLSSLTSEDSAVYYCARSTYYGGDWYFNVWGAGTTVTVSAASTKGPSVFPLAPCSRSTSGGTAAL
GCLVKDYFPEPVTVSWNSGALTSGVHTFPAVLQSSGLYSLSSVVTVPSSSLGTQTYTCNVNHKPSNTKVDKRV
ELKTPLGDTTHTCPRCPEPKSCDTPPPCPRCPEPKSCDTPPPCPRCPEPKSCDTPPPCPRCPAPELLGGPSVF
LFPPKPKDTLMISRTPEVTCVVVDVSHEDPEVQFKWYVDGVEVHNAKTKLREEQYNSTFRVVSVLTVLHQDWL
NGKEYKCKVSNKALPAPIEKTISKTKGQPREPQVYTLPPSREEMTKNQVSLTCLVKGFYPSDIAVEWESNGQP
ENNYKTTPPVLDSDGSFFLYSKLTVDKSRWQQGNIFSCSVMFEALHNHYTQKSLSLSPGK
SEQ ID NO: 173
Rit-IgG3-KVH HF (nucleotide sequence)
CAGGTTCAGCTGCAACAGCCTGGCGCCGAGCTTGTGAAACCTGGCGCCTCTGTGAAGATGAGCTGCAAGGCCA
GCGGCTACACCTTCACCAGCTACAACATGCACTGGGTCAAGCAGACCCCTGGCAGAGGCCTGGAATGGATCGG
AGCCATCTATCCCGGCAACGGCGACACCTCCTACAACCAGAAGTTCAAGGGCAAAGCCACACTGACCGCCGAC
AAGAGCAGCAGCACAGCCTACATGCAGCTGAGCAGCCTGACCAGCGAAGATAGCGCCGTGTACTACTGCGCCA
GAAGCACCTATTACGGCGGCGACTGGTACTTCAACGTGTGGGGAGCTGGTACCACCGTGACAGTTTCTGCCGC
TAGCACAAAAGGCCCTAGCGTCTTTCCACTCGCCCCATGTTCTAGAAGCACCTCTGGGGGCACTGCGGCTTTG
GGCTGCTTGGTGAAGGATTACTTCCCAGAGCCTGTCACTGTCAGCTGGAATAGCGGGGCTCTGACCAGTGGAG
TGCACACCTTTCCCGCCGTGTTACAGAGCAGCGGCCTCTATAGCCTGAGCAGTGTGGTAACTGTGCCCTCGAG
CAGCTTGGGCACCCAGACCTATACCTGCAATGTCAATCATAAACCCAGTAACACAAAGGTAGACAAGCGGGTC
GAACTGAAAACCCCCCTGGGCGACACCACCCACACCTGTCCTAGATGCCCCGAGCCCAAGTCCTGCGATACCC
CTCCACCTTGCCCCAGGTGCCCTGAGCCTAAGAGCTGTGATACCCCCCCTCCCTGCCCTCGGTGTCCAGAGCC
TAAGTCTTGTGACACCCCACCCCCATGTCCAAGGTGCCCCGCCCCCGAACTCCTGGGGGGACCTTCGGTTTTT
CTTTTTCCACCTAAACCGAAAGACACCCTGATGATCAGCAGGACACCAGAAGTGACATGTGTCGTCGTGGACG
TATCCCATGAGGACCCGGAGGTGCAGTTCAAGTGGTATGTTGATGGTGTGGAAGTGCATAATGCAAAAACTAA
GCTGCGGGAGGAACAGTACAATAGCACATTCAGAGTAGTTAGCGTTCTGACTGTCCTGCACCAAGATTGGCTG
AACGGGAAGGAATACAAGTGTAAGGTGAGCAACAAAGCCCTGCCCGCTCCAATTGAGAAAACAATTTCTAAGA
CCAAAGGACAGCCCCGGGAGCCACAGGTGTATACTCTGCCGCCTAGCAGGGAAGAGATGACCAAGAACCAGGT
GAGCCTGACCTGTCTGGTGAAAGGCTTCTACCCCAGCGATATCGCAGTTGAGTGGGAGAGCAATGGCCAACCC
GAGAACAACTACAAGACTACGCCCCCCGTGCTTGATAGCGACGGATCATTTTTCCTGTACTCAAAACTGACCG
TGGACAAAAGCAGATGGCAGCAGGGAAACATCTTCAGTTGCTCAGTGATGTTCGAAGCGCTGCACAATCATTA
TACTCAGAAAAGCCTGAGCTTGAGCCCGGGAAAA
SEQ ID NO: 174
Rit-IgG4 (amino acid sequence)
QVQLQQPGAELVKPGASVKMSCKASGYTFTSYNMHWVKQTPGRGLEWIGAIYPGNGDTSYNQKFKGKATLTAD
KSSSTAYMQLSSLTSEDSAVYYCARSTYYGGDWYFNVWGAGTTVTVSAASTKGPSVFPLAPCSRSTSESTAAL
GCLVKDYFPEPVTVSWNSGALTSGVHTFPAVLQSSGLYSLSSVVTVPSSSLGTKTYTCNVDHKPSNTKVDKRV
ESKYGPPCPSCPAPEFLGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQVYTLPPSQEEMTKNQVS
LTCLVKGFYPSDIAVEWESNGQPENNYKTTPPVLDSDGSFFLYSRLTVDKSRWQEGNVFSCSVMHEALHNHYT
QKSLSLSLGK
SEQ ID NO: 175
Rit-IgG4 (nucleotide sequence)
CAGGTTCAGCTGCAACAGCCTGGCGCCGAGCTTGTGAAACCTGGCGCCTCTGTGAAGATGAGCTGCAAGGCCA
GCGGCTACACCTTCACCAGCTACAACATGCACTGGGTCAAGCAGACCCCTGGCAGAGGCCTGGAATGGATCGG
AGCCATCTATCCCGGCAACGGCGACACCTCCTACAACCAGAAGTTCAAGGGCAAAGCCACACTGACCGCCGAC
AAGAGCAGCAGCACAGCCTACATGCAGCTGAGCAGCCTGACCAGCGAAGATAGCGCCGTGTACTACTGCGCCA
GAAGCACCTATTACGGCGGCGACTGGTACTTCAACGTGTGGGGAGCTGGTACCACCGTGACAGTTTCTGCCGC
TAGCACAAAAGGCCCTAGCGTCTTTCCACTCGCCCCATGTTCCAGAAGCACCTCTGAAAGTACTGCGGCTTTG
GGCTGCTTGGTGAAGGATTACTTCCCAGAGCCTGTCACTGTCAGCTGGAATAGCGGGGCTCTGACCAGTGGAG
TGCACACCTTTCCCGCCGTGTTACAGAGCAGCGGCCTCTATAGCCTGAGCAGTGTGGTAACTGTGCCCTCGAG
CAGCTTGGGCACCAAGACCTATACATGCAATGTCGACCATAAACCCAGTAACACAAAGGTAGACAAGCGAGTC
GAAAGCAAGTACGGCCCCCCCTGCCCTAGCTGCCCCGCCCCCGAATTTCTGGGGGGACCTTCGGTTTTTCTTT
TTCCACCTAAACCGAAAGACACCCTGATGATCAGCAGGACACCAGAAGTGACATGTGTCGTCGTGGACGTATC
CCAGGAGGACCCGGAGGTGCAGTTCAACTGGTATGTTGATGGTGTGGAAGTGCATAATGCAAAAACTAAGCCA
CGGGAGGAACAGTTCAATAGCACATACAGAGTAGTTAGCGTTCTGACTGTCCTGCACCAAGATTGGCTGAACG
GGAAGGAATACAAGTGTAAGGTGAGCAACAAAGGCCTGCCCAGTAGCATTGAGAAAACAATTTCTAAGGCCAA
AGGACAGCCCCGGGAGCCACAGGTGTATACTCTGCCGCCTAGCCAAGAGGAAATGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCGTGCTTGATAGCGACGGATCATTTTTCCTGTACTCACGGCTGACCGTGGA
CAAAAGCAGATGGCAGGAGGGAAACGTTTTCAGTTGCTCAGTGATGCACGAAGCGCTGCACAATCATTATACT
CAGAAAAGCCTGAGCTTGAGCTTAGGAAAA
SEQ ID NO: 176
Rit-IgG4 HF (amino acid sequence)
QVQLQQPGAELVKPGASVKMSCKASGYTFTSYNMHWVKQTPGRGLEWIGAIYPGNGDTSYNQKFKGKATLTAD
KSSSTAYMQLSSLTSEDSAVYYCARSTYYGGDWYFNVWGAGTTVTVSAASTKGPSVFPLAPCSRSTSESTAAL
GCLVKDYFPEPVTVSWNSGALTSGVHTFPAVLQSSGLYSLSSVVTVPSSSLGTKTYTCNVDHKPSNTKVDKRV
ESKYGPPCPSCPAPEFLGGPSVFLFPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAKGQPREPQVYTLPPSQEEMTKNQVS
LTCLVKGFYPSDIAVEWESNGQPENNYKTTPPVLDSDGSFFLYSRLTVDKSRWQEGNVFSCSVMFEALHNHYT
QKSLSLSLGK
SEQ ID NO: 177
Rit-IgG4 HF (nucleotide sequence)
CAGGTTCAGCTGCAACAGCCTGGCGCCGAGCTTGTGAAACCTGGCGCCTCTGTGAAGATGAGCTGCAAGGCCA
GCGGCTACACCTTCACCAGCTACAACATGCACTGGGTCAAGCAGACCCCTGGCAGAGGCCTGGAATGGATCGG
AGCCATCTATCCCGGCAACGGCGACACCTCCTACAACCAGAAGTTCAAGGGCAAAGCCACACTGACCGCCGAC
AAGAGCAGCAGCACAGCCTACATGCAGCTGAGCAGCCTGACCAGCGAAGATAGCGCCGTGTACTACTGCGCCA
GAAGCACCTATTACGGCGGCGACTGGTACTTCAACGTGTGGGGAGCTGGTACCACCGTGACAGTTTCTGCCGC
TAGCACAAAAGGCCCTAGCGTCTTTCCACTCGCCCCATGTTCCAGAAGCACCTCTGAAAGTACTGCGGCTTTG
GGCTGCTTGGTGAAGGATTACTTCCCAGAGCCTGTCACTGTCAGCTGGAATAGCGGGGCTCTGACCAGTGGAG
TGCACACCTTTCCCGCCGTGTTACAGAGCAGCGGCCTCTATAGCCTGAGCAGTGTGGTAACTGTGCCCTCGAG
CAGCTTGGGCACCAAGACCTATACATGCAATGTCGACCATAAACCCAGTAACACAAAGGTAGACAAGCGAGTC
GAAAGCAAGTACGGCCCCCCCTGCCCTAGCTGCCCCGCCCCCGAATTTCTGGGGGGACCTTCGGTTTTTCTTT
TTCCACCTAAACCGAAAGACACCCTGATGATCAGCAGGACACCAGAAGTGACATGTGTCGTCGTGGACGTATC
CCAGGAGGACCCGGAGGTGCAGTTCAACTGGTATGTTGATGGTGTGGAAGTGCATAATGCAAAAACTAAGCCA
CGGGAGGAACAGTTCAATAGCACATACAGAGTAGTTAGCGTTCTGACTGTCCTGCACCAAGATTGGCTGAACG
GGAAGGAATACAAGTGTAAGGTGAGCAACAAAGGCCTGCCCAGTAGCATTGAGAAAACAATTTCTAAGGCCAA
AGGACAGCCCCGGGAGCCACAGGTGTATACTCTGCCGCCTAGCCAAGAGGAAATGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCGTGCTTGATAGCGACGGATCATTTTTCCTGTACTCACGGCTGACCGTGGA
CAAAAGCAGATGGCAGGAGGGAAACGTTTTCAGTTGCTCAGTGATGttCGAAGCGCTGCACAATCATTATACT
CAGAAAAGCCTGAGCTTGAGCTTAGGAAAA
SEQ ID NO: 178 human IgG1 (amino acid sequence)
GQPREPQVYTLPPSRDELTKNQVSLTCLVKGFYPSDIAVEWESNGQPENNYKTTPPVLDSDGSFFLYSKL
TVDKSRWQQGNVFSCSVMHEALHNHYTQKSLSLSPGK
SEQ ID NO: 179 human IgG3 (amino acid sequence)
GQPREPQVYTLPPSREEMTKNQVSLTCLVKGFYPSDIAVEWESSGQPENNYNTTPPMLDS
DGSFFLYSKLTVDKSRWQQGNIFSCSVMHEALHNRFTQKSLSLSPGK
SEQ ID NO: 180 human IgG4 (amino acid sequence)
GQPREPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK
SEQ ID NO: 181 human IgG2 (amino acid sequence)
GQPREPQVYTLPPSREEMTKNQVSLTCLVKGFYPSDIAVEWESNGQPENNYKTTPPMLDS
DGSFFLYSKLTVDKSRWQQGNVFSCSVMHEALHNHYTQKSLSLSPGK
SEQ ID NO: 182 human IgAl (amino acid sequence)
GNTFRPEVHLLPPPSEELALNELVTLTCLARGESPKDVLVRWLQGSQELPREKYLTWASR
QEPSQGTTTFAVTSILRVAAEDWKKGDTFSCMVGHEALPLAFTQKTIDRLAGKPTHVNVS
VVMAEVDGTCY
SEQ ID NO: 183 human IgGA2 (amino acid sequence)
GNTFRPEVHLLPPPSEELALNELVTLTCLARGFSPKDVLVRWLQGSQELPREKYLTWASR
QEPSQGTTTFAVTSILRVAAEDWKKGDTFSCMVGHEALPLAFTQKTIDRLAGKPTHVNVS
VVMAEVDGTCY
SEQ ID 184: Isa-IgG2-WT: Isatuximab IgG2 WT Heavy Chain (Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPCSRSTSESTAALG
CLVKDYFPEPVTVSWNSGALTSGVHTFPAVLQSSGLYSLSSVVTVPSSNFGTQTYTCNVDHKPSNTKVDKTVE
RKCCVECPPCPAPPVAGPSVFLFPPKPKDTLMISRTPEVTCVVVDVSHEDPEVQFNWYVDGVEVHNAKTKPRE
EQFNSTFRVVSVLTVVHQDWLNGKEYKCKVSNKGLPAPIEKTISKTKGQPREPQVYTLPPSREEMTKNQVSLT
CLVKGFYPSDIAVEWESNGQPENNYKTTPPMLDSDGSFFLYSKLTVDKSRWQQGNVFSCSVMHEALHNHYTQK
SLSLSPG*
SEQ ID 185: Isa-IgG2-WT: Isatuximab IgG2 WT Heavy Chain (DNA)
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGTACCTCCGTCACAGTTAGCTCTGCTAG
CACAAAAGGCCCTAGCGTCTTTCCACTCGCCCCATGTTCAAGAAGCACCAGCGAGTCAACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAA
TTTTGGCACCCAGACCTATACATGCAATGTCGATCATAAACCCAGTAACACAAAGGTAGACAAGACAGTCGAA
AGAAAATGCTGTGTGGAGTGCCCTCCTTGCCCCGCCCCCCCAGTGGCCGGGCCTTCGGTTTTTCTTTTTCCAC
CTAAACCGAAAGACACCCTGATGATCAGCAGGACACCAGAAGTGACATGTGTCGTCGTGGACGTATCCCATGA
AGACCCGGAGGTGCAATTCAACTGGTATGTTGATGGTGTGGAAGTGCATAATGCAAAAACTAAGCCACGGGAG
GAACAGTTCAATAGCACATTCAGAGTAGTTAGCGTTCTGACTGTCGTGCACCAAGATTGGCTGAACGGGAAGG
AATACAAGTGTAAGGTGAGCAACAAAGGTCTGCCCGCTCCAATTGAGAAAACAATTTCTAAGACCAAAGGACA
GCCCCGGGAGCCACAGGTGTATACTCTGCCGCCTAGCAGGGAGGAAATGACCAAGAACCAGGTGAGCCTGACC
TGTCTGGTGAAAGGCTTCTACCCCAGCGATATCGCAGTTGAGTGGGAGAGCAATGGCCAACCCGAGAACAACT
ACAAGACTACGCCCCCCATGCTTGATAGCGACGGATCATTTTTCCTGTACTCAAAACTGACCGTGGACAAAAG
CAGATGGCAGCAGGGAAACGTTTTCAGTTGCTCAGTGATGCACGAAGCGCTGCACAATCATTATACTCAGAAA
AGCCTGAGCTTGAGCCCGGGATGA
SEQ ID 186: Isa-IgG2-M1: Isatuximab IgG2 C131S C220S EFLGG Heavy Chain
(Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPSSRSTSESTAALG
CLVKDYFPEPVTVSWNSGALTSGVHTFPAVLQSSGLYSLSSVVTVPSSNFGTQTYTCNVDHKPSNTKVDKTVE
RKCSVECPPCPAPEFLGGPSVFLFPPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKGQPREPQVYTLPPSREEMTKNQVSL
TCLVKGFYPSDIAVEWESNGQPENNYKTTPPMLDSDGSFFLYSKLTVDKSRWQQGNVFSCSVMHEALHNHYTQ
KSLSLSPG*
SEQ ID 187: Isa-IgG2-M1: Isatuximab IgG2 C131S C220S EFLGG Heavy Chain
(DNA)
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGTACCTCCGTCACAGTTAGCTCTGCTAG
CACAAAAGGCCCTAGCGTCTTTCCACTCGCCCCAAGCTCAAGAAGCACCAGCGAGTCAACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAA
TTTTGGCACCCAGACCTATACATGCAATGTCGATCATAAACCCAGTAACACAAAGGTAGACAAGACAGTCGAA
AGAAAATGCAGCGTGGAGTGCCCTCCTTGCCCCGCCCCCGAATTCCTGGGCGGCCCTTCGGTTTTTCTTTTTC
CACCTAAACCGAAAGACACCCTGATGATCAGCAGGACACCAGAAGTGACATGTGTCGTCGTGGACGTATCCCA
TGAAGACCCGGAGGTGCAATTCAACTGGTATGTTGATGGTGTGGAAGTGCATAATGCAAAAACTAAGCCACGG
GAGGAACAGTTCAATAGCACATTCAGAGTAGTTAGCGTTCTGACTGTCGTGCACCAAGATTGGCTGAACGGGA
AGGAATACAAGTGTAAGGTGAGCAACAAAGGTCTGCCCGCTCCAATTGAGAAAACAATTTCTAAGACCAAAGG
ACAGCCCCGGGAGCCACAGGTGTATACTCTGCCGCCTAGCAGGGAGGAAATGACCAAGAACCAGGTGAGCCTG
ACCTGTCTGGTGAAAGGCTTCTACCCCAGCGATATCGCAGTTGAGTGGGAGAGCAATGGCCAACCCGAGAACA
ACTACAAGACTACGCCCCCCATGCTTGATAGCGACGGATCATTTTTCCTGTACTCAAAACTGACCGTGGACAA
AAGCAGATGGCAGCAGGGAAACGTTTTCAGTTGCTCAGTGATGCACGAAGCGCTGCACAATCATTATACTCAG
AAAAGCCTGAGCTTGAGCCCGGGATGA
SEQ ID 188: Isa-IgG2-M2: Isatuximab IgG2 C131S C219S EFLGG Heavy Chain
(Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPSSRSTSESTAALG
CLVKDYFPEPVTVSWNSGALTSGVHTFPAVLQSSGLYSLSSVVTVPSSNFGTQTYTCNVDHKPSNTKVDKTVE
RKSCVECPPCPAPEFLGGPSVFLFPPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKGQPREPQVYTLPPSREEMTKNQVSL
TCLVKGFYPSDIAVEWESNGQPENNYKTTPPMLDSDGSFFLYSKLTVDKSRWQQGNVFSCSVMHEALHNHYTQ
KSLSLSPG*
SEQ ID 189: Isa-IgG2-M2: Isatuximab IgG2 C131S C219S EFLGG Heavy Chain
(DNA)
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGTACCTCCGTCACAGTTAGCTCTGCTAG
CACAAAAGGCCCTAGCGTCTTTCCACTCGCCCCAAGCTCAAGAAGCACCAGCGAGTCAACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAA
TTTTGGCACCCAGACCTATACATGCAATGTCGATCATAAACCCAGTAACACAAAGGTAGACAAGACAGTCGAA
AGAAAAAGCTGCGTGGAGTGCCCTCCTTGCCCCGCCCCCGAATTCCTGGGCGGCCCTTCGGTTTTTCTTTTTC
CACCTAAACCGAAAGACACCCTGATGATCAGCAGGACACCAGAAGTGACATGTGTCGTCGTGGACGTATCCCA
TGAAGACCCGGAGGTGCAATTCAACTGGTATGTTGATGGTGTGGAAGTGCATAATGCAAAAACTAAGCCACGG
GAGGAACAGTTCAATAGCACATTCAGAGTAGTTAGCGTTCTGACTGTCGTGCACCAAGATTGGCTGAACGGGA
AGGAATACAAGTGTAAGGTGAGCAACAAAGGTCTGCCCGCTCCAATTGAGAAAACAATTTCTAAGACCAAAGG
ACAGCCCCGGGAGCCACAGGTGTATACTCTGCCGCCTAGCAGGGAGGAAATGACCAAGAACCAGGTGAGCCTG
ACCTGTCTGGTGAAAGGCTTCTACCCCAGCGATATCGCAGTTGAGTGGGAGAGCAATGGCCAACCCGAGAACA
ACTACAAGACTACGCCCCCCATGCTTGATAGCGACGGATCATTTTTCCTGTACTCAAAACTGACCGTGGACAA
AAGCAGATGGCAGCAGGGAAACGTTTTCAGTTGCTCAGTGATGCACGAAGCGCTGCACAATCATTATACTCAG
AAAAGCCTGAGCTTGAGCCCGGGATGA
SEQ ID 190: Isa-IgG2-M3: Isatuximab IgG2 uH1 EFLGG Heavy Chain (Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPSSRSTSESTAALG
CLVKDYFPEPVTVSWNSGALTSGVHTFPAVLQSSGLYSLSSVVTVPSSNFGTQTYTCNVDHKPSNTKVDKTVE
PKSCDKTHTCPPCPAPEFLGGPSVFLFPPKPKDTLMISRTPEVTCVVVDVSHEDPEVQFNWYVDGVEVHNAKT
KPREEQFNSTFRVVSVLTVVHQDWLNGKEYKCKVSNKGLPAPIEKTISKTKGQPREPQVYTLPPSREEMTKNQ
VSLTCLVKGFYPSDIAVEWESNGQPENNYKTTPPMLDSDGSFFLYSKLTVDKSRWQQGNVFSCSVMHEALHNH
YTQKSLSLSPG*
SEQ ID 191: Isa-IgG2-M3: Isatuximab IgG2 uH1 EFLGG Heavy Chain (DNA)
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGTACCTCCGTCACAGTTAGCTCTGCTAG
CACAAAAGGCCCTAGCGTCTTTCCACTCGCCCCAAGCTCAAGAAGCACCAGCGAGTCAACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAA
TTTTGGCACCCAGACCTATACATGCAATGTCGATCATAAACCCAGTAACACAAAGGTAGACAAGACAGTCGAA
CCCAAGAGCTGCGACAAGACCCACACATGTCCACCATGCCCCGCCCCCGAATTCCTGGGCGGCCCTTCGGTTT
TTCTTTTTCCACCTAAACCGAAAGACACCCTGATGATCAGCAGGACACCAGAAGTGACATGTGTCGTCGTGGA
CGTATCCCATGAAGACCCGGAGGTGCAATTCAACTGGTATGTTGATGGTGTGGAAGTGCATAATGCAAAAACT
AAGCCACGGGAGGAACAGTTCAATAGCACATTCAGAGTAGTTAGCGTTCTGACTGTCGTGCACCAAGATTGGC
TGAACGGGAAGGAATACAAGTGTAAGGTGAGCAACAAAGGTCTGCCCGCTCCAATTGAGAAAACAATTTCTAA
GACCAAAGGACAGCCCCGGGAGCCACAGGTGTATACTCTGCCGCCTAGCAGGGAGGAAATGACCAAGAACCAG
GTGAGCCTGACCTGTCTGGTGAAAGGCTTCTACCCCAGCGATATCGCAGTTGAGTGGGAGAGCAATGGCCAAC
CCGAGAACAACTACAAGACTACGCCCCCCATGCTTGATAGCGACGGATCATTTTTCCTGTACTCAAAACTGAC
CGTGGACAAAAGCAGATGGCAGCAGGGAAACGTTTTCAGTTGCTCAGTGATGCACGAAGCGCTGCACAATCAT
TATACTCAGAAAAGCCTGAGCTTGAGCCCGGGATGA
SEQ ID 192: Isa-IgG2-C131S-C219S: Isatuximab IgG2 C131S C219S Heavy Chain
(Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPSSRSTSESTAALG
CLVKDYFPEPVTVSWNSGALTSGVHTFPAVLQSSGLYSLSSVVTVPSSNFGTQTYTCNVDHKPSNTKVDKTVE
RKSCVECPPCPAPPVAGPSVFLFPPKPKDTLMISRTPEVTCVVVDVSHEDPEVQFNWYVDGVEVHNAKTKPRE
EQFNSTFRVVSVLTVVHQDWLNGKEYKCKVSNKGLPAPIEKTISKTKGQPREPQVYTLPPSREEMTKNQVSLT
CLVKGFYPSDIAVEWESNGQPENNYKTTPPMLDSDGSFFLYSKLTVDKSRWQQGNVFSCSVMHEALHNHYTQK
SLSLSPG*
SEQ ID 193: Isa-IgG2-C131S-C219S: Isatuximab IgG2 C131S C219S Heavy Chain
(DNA)
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGTACCTCCGTCACAGTTAGCTCTGCTAG
CACAAAAGGCCCTAGCGTCTTTCCACTCGCCCCAAGCTCAAGAAGCACCAGCGAGTCAACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAA
TTTTGGCACCCAGACCTATACATGCAATGTCGATCATAAACCCAGTAACACAAAGGTAGACAAGACAGTCGAA
AGAAAAAGCTGCGTGGAGTGCCCTCCTTGCCCCGCCCCCCCAGTGGCCGGCCCTTCGGTTTTTCTTTTTCCAC
CTAAACCGAAAGACACCCTGATGATCAGCAGGACACCAGAAGTGACATGTGTCGTCGTGGACGTATCCCATGA
AGACCCGGAGGTGCAATTCAACTGGTATGTTGATGGTGTGGAAGTGCATAATGCAAAAACTAAGCCACGGGAG
GAACAGTTCAATAGCACATTCAGAGTAGTTAGCGTTCTGACTGTCGTGCACCAAGATTGGCTGAACGGGAAGG
AATACAAGTGTAAGGTGAGCAACAAAGGTCTGCCCGCTCCAATTGAGAAAACAATTTCTAAGACCAAAGGACA
GCCCCGGGAGCCACAGGTGTATACTCTGCCGCCTAGCAGGGAGGAAATGACCAAGAACCAGGTGAGCCTGACC
TGTCTGGTGAAAGGCTTCTACCCCAGCGATATCGCAGTTGAGTGGGAGAGCAATGGCCAACCCGAGAACAACT
ACAAGACTACGCCCCCCATGCTTGATAGCGACGGATCATTTTTCCTGTACTCAAAACTGACCGTGGACAAAAG
CAGATGGCAGCAGGGAAACGTTTTCAGTTGCTCAGTGATGCACGAAGCGCTGCACAATCATTATACTCAGAAA
AGCCTGAGCTTGAGCCCGGGATGA
SEQ ID 194: Isa-IgG2-EFLGG: Isatuximab IgG2 EFLGG Heavy Chain (Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPCSRSTSESTAALG
CLVKDYFPEPVTVSWNSGALTSGVHTFPAVLQSSGLYSLSSVVTVPSSNFGTQTYTCNVDHKPSNTKVDKTVE
RKCCVECPPCPAPEFLGGPSVFLFPPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKGQPREPQVYTLPPSREEMTKNQVSL
TCLVKGFYPSDIAVEWESNGQPENNYKTTPPMLDSDGSFFLYSKLTVDKSRWQQGNVFSCSVMHEALHNHYTQ
KSLSLSPG*
SEQ ID 195: Isa-IgG2-EFLGG: Isatuximab IgG2 EFLGG Heavy Chain (DNA)
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGTACCTCCGTCACAGTTAGCTCTGCTAG
CACAAAAGGCCCTAGCGTCTTTCCACTCGCCCCATGTTCAAGAAGCACCAGCGAGTCAACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAA
TTTTGGCACCCAGACCTATACATGCAATGTCGATCATAAACCCAGTAACACAAAGGTAGACAAGACAGTCGAA
AGAAAATGCTGTGTGGAGTGCCCTCCTTGCCCCGCCCCCGAATTCCTGGGCGGCCCTTCGGTTTTTCTTTTTC
CACCTAAACCGAAAGACACCCTGATGATCAGCAGGACACCAGAAGTGACATGTGTCGTCGTGGACGTATCCCA
TGAAGACCCGGAGGTGCAATTCAACTGGTATGTTGATGGTGTGGAAGTGCATAATGCAAAAACTAAGCCACGG
GAGGAACAGTTCAATAGCACATTCAGAGTAGTTAGCGTTCTGACTGTCGTGCACCAAGATTGGCTGAACGGGA
AGGAATACAAGTGTAAGGTGAGCAACAAAGGTCTGCCCGCTCCAATTGAGAAAACAATTTCTAAGACCAAAGG
ACAGCCCCGGGAGCCACAGGTGTATACTCTGCCGCCTAGCAGGGAGGAAATGACCAAGAACCAGGTGAGCCTG
ACCTGTCTGGTGAAAGGCTTCTACCCCAGCGATATCGCAGTTGAGTGGGAGAGCAATGGCCAACCCGAGAACA
ACTACAAGACTACGCCCCCCATGCTTGATAGCGACGGATCATTTTTCCTGTACTCAAAACTGACCGTGGACAA
AAGCAGATGGCAGCAGGGAAACGTTTTCAGTTGCTCAGTGATGCACGAAGCGCTGCACAATCATTATACTCAG
AAAAGCCTGAGCTTGAGCCCGGGATGA
SEQ ID 196: Isatuximab IgG1 H429F Heavy Chain (Protein)
QVQLQQPGAELVKPGASVKMSCKASGYTFTSYNMHWVKQTPGRGLEWIGAIYPGNGDTSYNQKFKGKATLTAD
KSSSTAYMQLSSLTSEDSAVYYCARSTYYGGDWYFNVWGAGTTVTVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGNVFSCSVMFEALHN
HYTQKSLSLSPGK*
SEQ ID 197: Isatuximab IgG1 H429F Heavy Chain (DNA)
CAGGTTCAGCTGCAACAGCCTGGCGCCGAGCTTGTGAAACCTGGCGCCTCTGTGAAGATGAGCTGCAAGGCCA
GCGGCTACACCTTCACCAGCTACAACATGCACTGGGTCAAGCAGACCCCTGGCAGAGGCCTGGAATGGATCGG
AGCCATCTATCCCGGCAACGGCGACACCTCCTACAACCAGAAGTTCAAGGGCAAAGCCACACTGACCGCCGAC
AAGAGCAGCAGCACAGCCTACATGCAGCTGAGCAGCCTGACCAGCGAAGATAGCGCCGTGTACTACTGCGCCA
GAAGCACCTATTACGGCGGCGACTGGTACTTCAACGTGTGGGGAGCTGGCACCACCGTGACAGTTTCTGCCGC
TAGCACAAAAGGCCCTAGCGTCTTTCCACTCGCCCCATCCTCCAAGAGCACCTCTGGGGGCACTGCGGCTTTG
GGCTGCTTGGTGAAGGATTACTTCCCAGAGCCTGTCACTGTCAGCTGGAATAGCGGGGCTCTGACCAGTGGAG
TGCACACCTTTCCCGCCGTGTTACAGAGCAGCGGCCTCTATAGCCTGAGCAGTGTGGTAACTGTGCCCTCGAG
CAGCTTGGGTACCCAGACCTATATCTGCAATGTCAATCATAAACCCAGTAACACAAAGGTAGACAAGAAAGTC
GAACCCAAATCTTGCGACAAAACTCACACATGCCCTCCTTGCCCCGCCCCCGAACTCCTGGGGGGACCTTCGG
TTTTTCTTTTTCCACCTAAACCGAAAGACACCCTGATGATCAGCAGGACACCAGAAGTGACATGTGTCGTCGT
GGACGTATCCCATGAGGACCCGGAGGTGAAGTTCAACTGGTATGTTGATGGTGTGGAAGTGCATAATGCAAAA
ACTAAGCCACGGGAGGAACAGTACAATAGCACATACAGAGTAGTTAGCGTTCTGACTGTCCTGCACCAAGATT
GGCTGAACGGGAAGGAATACAAGTGTAAGGTGAGCAACAAAGCCCTGCCCGCTCCAATTGAGAAAACAATTTC
TAAGGCCAAAGGACAGCCCCGGGAGCCACAGGTGTATACTCTGCCGCCTAGCAGGGATGAACTGACCAAGAAC
CAGGTGAGCCTGACCTGTCTGGTGAAAGGCTTCTACCCCAGCGATATCGCAGTTGAGTGGGAGAGCAATGGCC
AACCCGAGAACAACTACAAGACTACGCCCCCCGTGCTTGATAGCGACGGATCATTTTTCCTGTACTCAAAACT
GACCGTGGACAAAAGCAGATGGCAGCAGGGAAACGTTTTCAGTTGCTCAGTGATGttCGAAGCGCTGCACAAT
CATTATACTCAGAAAAGCCTGAGCTTGAGCCCGGGAAAATGA
SEQ ID 198: Isatuximab IgG2 H429F Heavy Chain (Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPCSRSTSESTAALG
CLVKDYFPEPVTVSWNSGALTSGVHTFPAVLQSSGLYSLSSVVTVPSSNFGTQTYTCNVDHKPSNTKVDKTVE
RKCCVECPPCPAPPVAGPSVFLFPPKPKDTLMISRTPEVTCVVVDVSHEDPEVQFNWYVDGVEVHNAKTKPRE
EQFNSTFRVVSVLTVVHQDWLNGKEYKCKVSNKGLPAPIEKTISKTKGQPREPQVYTLPPSREEMTKNQVSLT
CLVKGFYPSDIAVEWESNGQPENNYKTTPPMLDSDGSFFLYSKLTVDKSRWQQGNVFSCSVMFEALHNHYTQK
SLSLSPG*
SEQ ID 199 Isatuximab IgG2 H429F Heavy Chain (DNA)
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGTACCTCCGTCACAGTTAGCTCTGCTAG
CACAAAAGGCCCTAGCGTCTTTCCACTCGCCCCATGTTCAAGAAGCACCAGCGAGTCAACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAA
TTTTGGCACCCAGACCTATACATGCAATGTCGATCATAAACCCAGTAACACAAAGGTAGACAAGACAGTCGAA
AGAAAATGCTGTGTGGAGTGCCCTCCTTGCCCCGCCCCCCCAGTGGCCGGGCCTTCGGTTTTTCTTTTTCCAC
CTAAACCGAAAGACACCCTGATGATCAGCAGGACACCAGAAGTGACATGTGTCGTCGTGGACGTATCCCATGA
AGACCCGGAGGTGCAATTCAACTGGTATGTTGATGGTGTGGAAGTGCATAATGCAAAAACTAAGCCACGGGAG
GAACAGTTCAATAGCACATTCAGAGTAGTTAGCGTTCTGACTGTCGTGCACCAAGATTGGCTGAACGGGAAGG
AATACAAGTGTAAGGTGAGCAACAAAGGTCTGCCCGCTCCAATTGAGAAAACAATTTCTAAGACCAAAGGACA
GCCCCGGGAGCCACAGGTGTATACTCTGCCGCCTAGCAGGGAGGAAATGACCAAGAACCAGGTGAGCCTGACC
TGTCTGGTGAAAGGCTTCTACCCCAGCGATATCGCAGTTGAGTGGGAGAGCAATGGCCAACCCGAGAACAACT
ACAAGACTACGCCCCCCATGCTTGATAGCGACGGATCATTTTTCCTGTACTCAAAACTGACCGTGGACAAAAG
CAGATGGCAGCAGGGAAACGTTTTCAGTTGCTCAGTGATGTTCGAAGCGCTGCACAATCATTATACTCAGAAA
AGCCTGAGCTTGAGCCCGGGATGA
SEQ ID 200: Isatuximab IgG2 M1 H429F Heavy Chain (Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPSSRSTSESTAALG
CLVKDYFPEPVTVSWNSGALTSGVHTFPAVLQSSGLYSLSSVVTVPSSNFGTQTYTCNVDHKPSNTKVDKTVE
RKCSVECPPCPAPEFLGGPSVFLFPPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKGQPREPQVYTLPPSREEMTKNQVSL
TCLVKGFYPSDIAVEWESNGQPENNYKTTPPMLDSDGSFFLYSKLTVDKSRWQQGNVFSCSVMFEALHNHYTQ
KSLSLSPG*
SEQ ID 201: Isatuximab IgG2 M1 H429F Heavy Chain (DNA)
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGTACCTCCGTCACAGTTAGCTCTGCTAG
CACAAAAGGCCCTAGCGTCTTTCCACTCGCCCCAAGCTCAAGAAGCACCAGCGAGTCAACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAA
TTTTGGCACCCAGACCTATACATGCAATGTCGATCATAAACCCAGTAACACAAAGGTAGACAAGACAGTCGAA
AGAAAATGCAGCGTGGAGTGCCCTCCTTGCCCCGCCCCCGAATTCCTGGGCGGCCCTTCGGTTTTTCTTTTTC
CACCTAAACCGAAAGACACCCTGATGATCAGCAGGACACCAGAAGTGACATGTGTCGTCGTGGACGTATCCCA
TGAAGACCCGGAGGTGCAATTCAACTGGTATGTTGATGGTGTGGAAGTGCATAATGCAAAAACTAAGCCACGG
GAGGAACAGTTCAATAGCACATTCAGAGTAGTTAGCGTTCTGACTGTCGTGCACCAAGATTGGCTGAACGGGA
AGGAATACAAGTGTAAGGTGAGCAACAAAGGTCTGCCCGCTCCAATTGAGAAAACAATTTCTAAGACCAAAGG
ACAGCCCCGGGAGCCACAGGTGTATACTCTGCCGCCTAGCAGGGAGGAAATGACCAAGAACCAGGTGAGCCTG
ACCTGTCTGGTGAAAGGCTTCTACCCCAGCGATATCGCAGTTGAGTGGGAGAGCAATGGCCAACCCGAGAACA
ACTACAAGACTACGCCCCCCATGCTTGATAGCGACGGATCATTTTTCCTGTACTCAAAACTGACCGTGGACAA
AAGCAGATGGCAGCAGGGAAACGTTTTCAGTTGCTCAGTGATGTTCGAAGCGCTGCACAATCATTATACTCAG
AAAAGCCTGAGCTTGAGCCCGGGATGA
SEQ ID 202: Isatuximab IgG2 M2 H429F Heavy Chain (Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPSSRSTSESTAALG
CLVKDYFPEPVTVSWNSGALTSGVHTFPAVLQSSGLYSLSSVVTVPSSNFGTQTYTCNVDHKPSNTKVDKTVE
RKSCVECPPCPAPEFLGGPSVFLFPPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKGQPREPQVYTLPPSREEMTKNQVSL
TCLVKGFYPSDIAVEWESNGQPENNYKTTPPMLDSDGSFFLYSKLTVDKSRWQQGNVFSCSVMFEALHNHYTQ
KSLSLSPG*
SEQ ID 203: Isatuximab IgG2 M2 H429F Heavy Chain (DNA)
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGTACCTCCGTCACAGTTAGCTCTGCTAG
CACAAAAGGCCCTAGCGTCTTTCCACTCGCCCCAAGCTCAAGAAGCACCAGCGAGTCAACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAA
TTTTGGCACCCAGACCTATACATGCAATGTCGATCATAAACCCAGTAACACAAAGGTAGACAAGACAGTCGAA
AGAAAAAGCTGCGTGGAGTGCCCTCCTTGCCCCGCCCCCGAATTCCTGGGCGGCCCTTCGGTTTTTCTTTTTC
CACCTAAACCGAAAGACACCCTGATGATCAGCAGGACACCAGAAGTGACATGTGTCGTCGTGGACGTATCCCA
TGAAGACCCGGAGGTGCAATTCAACTGGTATGTTGATGGTGTGGAAGTGCATAATGCAAAAACTAAGCCACGG
GAGGAACAGTTCAATAGCACATTCAGAGTAGTTAGCGTTCTGACTGTCGTGCACCAAGATTGGCTGAACGGGA
AGGAATACAAGTGTAAGGTGAGCAACAAAGGTCTGCCCGCTCCAATTGAGAAAACAATTTCTAAGACCAAAGG
ACAGCCCCGGGAGCCACAGGTGTATACTCTGCCGCCTAGCAGGGAGGAAATGACCAAGAACCAGGTGAGCCTG
ACCTGTCTGGTGAAAGGCTTCTACCCCAGCGATATCGCAGTTGAGTGGGAGAGCAATGGCCAACCCGAGAACA
ACTACAAGACTACGCCCCCCATGCTTGATAGCGACGGATCATTTTTCCTGTACTCAAAACTGACCGTGGACAA
AAGCAGATGGCAGCAGGGAAACGTTTTCAGTTGCTCAGTGATGTTCGAAGCGCTGCACAATCATTATACTCAG
AAAAGCCTGAGCTTGAGCCCGGGATGA
SEQ ID 204 Isatuximab IgG2 M3 H429F Heavy Chain (Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPSSRSTSESTAALG
CLVKDYFPEPVTVSWNSGALTSGVHTFPAVLQSSGLYSLSSVVTVPSSNFGTQTYTCNVDHKPSNTKVDKTVE
PKSCDKTHTCPPCPAPEFLGGPSVFLFPPKPKDTLMISRTPEVTCVVVDVSHEDPEVQFNWYVDGVEVHNAKT
KPREEQFNSTFRVVSVLTVVHQDWLNGKEYKCKVSNKGLPAPIEKTISKTKGQPREPQVYTLPPSREEMTKNQ
VSLTCLVKGFYPSDIAVEWESNGQPENNYKTTPPMLDSDGSFFLYSKLTVDKSRWQQGNVFSCSVMFEALHNH
YTQKSLSLSPG*
SEQ ID 205: Isatuximab IgG2 M3 H429F Heavy Chain (DNA)
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGTACCTCCGTCACAGTTAGCTCTGCTAG
CACAAAAGGCCCTAGCGTCTTTCCACTCGCCCCAAGCTCAAGAAGCACCAGCGAGTCAACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAA
TTTTGGCACCCAGACCTATACATGCAATGTCGATCATAAACCCAGTAACACAAAGGTAGACAAGACAGTCGAA
CCCAAGAGCTGCGACAAGACCCACACATGTCCACCATGCCCCGCCCCCGAATTCCTGGGCGGCCCTTCGGTTT
TTCTTTTTCCACCTAAACCGAAAGACACCCTGATGATCAGCAGGACACCAGAAGTGACATGTGTCGTCGTGGA
CGTATCCCATGAAGACCCGGAGGTGCAATTCAACTGGTATGTTGATGGTGTGGAAGTGCATAATGCAAAAACT
AAGCCACGGGAGGAACAGTTCAATAGCACATTCAGAGTAGTTAGCGTTCTGACTGTCGTGCACCAAGATTGGC
TGAACGGGAAGGAATACAAGTGTAAGGTGAGCAACAAAGGTCTGCCCGCTCCAATTGAGAAAACAATTTCTAA
GACCAAAGGACAGCCCCGGGAGCCACAGGTGTATACTCTGCCGCCTAGCAGGGAGGAAATGACCAAGAACCAG
GTGAGCCTGACCTGTCTGGTGAAAGGCTTCTACCCCAGCGATATCGCAGTTGAGTGGGAGAGCAATGGCCAAC
CCGAGAACAACTACAAGACTACGCCCCCCATGCTTGATAGCGACGGATCATTTTTCCTGTACTCAAAACTGAC
CGTGGACAAAAGCAGATGGCAGCAGGGAAACGTTTTCAGTTGCTCAGTGATGTTCGAAGCGCTGCACAATCAT
TATACTCAGAAAAGCCTGAGCTTGAGCCCGGGATGA
SEQ ID 206: Isatuximab IgG2 EFLGG H429F Heavy Chain (Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPCSRSTSESTAALG
CLVKDYFPEPVTVSWNSGALTSGVHTFPAVLQSSGLYSLSSVVTVPSSNFGTQTYTCNVDHKPSNTKVDKTVE
RKCCVECPPCPAPEFLGGPSVFLFPPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKGQPREPQVYTLPPSREEMTKNQVSL
TCLVKGFYPSDIAVEWESNGQPENNYKTTPPMLDSDGSFFLYSKLTVDKSRWQQGNVFSCSVMFEALHNHYTQ
KSLSLSPG*
SEQ ID 207: Isatuximab IgG2 EFLGG H429F Heavy Chain (DNA)
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGTACCTCCGTCACAGTTAGCTCTGCTAG
CACAAAAGGCCCTAGCGTCTTTCCACTCGCCCCATGTTCAAGAAGCACCAGCGAGTCAACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAA
TTTTGGCACCCAGACCTATACATGCAATGTCGATCATAAACCCAGTAACACAAAGGTAGACAAGACAGTCGAA
AGAAAATGCTGTGTGGAGTGCCCTCCTTGCCCCGCCCCCGAATTCCTGGGCGGCCCTTCGGTTTTTCTTTTTC
CACCTAAACCGAAAGACACCCTGATGATCAGCAGGACACCAGAAGTGACATGTGTCGTCGTGGACGTATCCCA
TGAAGACCCGGAGGTGCAATTCAACTGGTATGTTGATGGTGTGGAAGTGCATAATGCAAAAACTAAGCCACGG
GAGGAACAGTTCAATAGCACATTCAGAGTAGTTAGCGTTCTGACTGTCGTGCACCAAGATTGGCTGAACGGGA
AGGAATACAAGTGTAAGGTGAGCAACAAAGGTCTGCCCGCTCCAATTGAGAAAACAATTTCTAAGACCAAAGG
ACAGCCCCGGGAGCCACAGGTGTATACTCTGCCGCCTAGCAGGGAGGAAATGACCAAGAACCAGGTGAGCCTG
ACCTGTCTGGTGAAAGGCTTCTACCCCAGCGATATCGCAGTTGAGTGGGAGAGCAATGGCCAACCCGAGAACA
ACTACAAGACTACGCCCCCCATGCTTGATAGCGACGGATCATTTTTCCTGTACTCAAAACTGACCGTGGACAA
AAGCAGATGGCAGCAGGGAAACGTTTTCAGTTGCTCAGTGATGTTCGAAGCGCTGCACAATCATTATACTCAG
AAAAGCCTGAGCTTGAGCCCGGGATGA
SEQ ID 208: Isatuximab IgG1 LALA Heavy Chain (Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVE
PKSCDKTHTCPPCPAPEAAGGPSVFLFPPKPKDTLMISRTPEVTCVVVDVSHEDPEVKENWYVDGVEVHNAKT
KPREEQYNSTYRVVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVFSCSVMHEALHNH
YTQKSLSLSPGK*
SEQ ID 209: Isatuximab IgG1 LALA Heavy Chain (DNA)
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGTACCTCCGTCACAGTTAGCTCTGCTAG
CACAAAAGGCCCTAGCGTCTTTCCACTCGCCCCATCCTCCAAGAGCACCTCTGGGGGCACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAG
CTTGGGTACCCAGACCTATATCTGCAATGTCAATCATAAACCCAGTAACACAAAGGTAGACAAGAAAGTCGAA
CCCAAATCTTGCGACAAAACTCACACATGCCCTCCTTGCCCCGCCCCCGAAGCCGCCGGGGGACCTTCGGTTT
TTCTTTTTCCACCTAAACCGAAAGACACCCTGATGATCAGCAGGACACCAGAAGTGACATGTGTCGTCGTGGA
CGTATCCCATGAGGACCCGGAGGTGAAGTTCAACTGGTATGTTGATGGTGTGGAAGTGCATAATGCAAAAACT
AAGCCACGGGAGGAACAGTACAATAGCACATACAGAGTAGTTAGCGTTCTGACTGTCCTGCACCAAGATTGGC
TGAACGGGAAGGAATACAAGTGTAAGGTGAGCAACAAAGCCCTGCCCGCTCCAATTGAGAAAACAATTTCTAA
GGCCAAAGGACAGCCCCGGGAGCCACAGGTGTATACTCTGCCGCCTAGCAGGGAGGAAATGACCAAGAACCAG
GTGAGCCTGACCTGTCTGGTGAAAGGCTTCTACCCCAGCGATATCGCAGTTGAGTGGGAGAGCAATGGCCAAC
CCGAGAACAACTACAAGACTACGCCCCCCGTGCTTGATAGCGACGGATCATTTTTCCTGTACTCAAAACTGAC
CGTGGACAAAAGCAGATGGCAGCAGGGAAACGTTTTCAGTTGCTCAGTGATGCACGAAGCGCTGCACAATCAT
TATACTCAGAAAAGCCTGAGCTTGAGCCCGGGAAAATGA
SEQ ID 210: Isatuximab IgG1 LALA H429F Heavy Chain (Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVE
PKSCDKTHTCPPCPAPEAAGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVFSCSVMFEALHNH
YTQKSLSLSPGK*
SEQ ID 211: Isatuximab IgG1 LALA H429F Heavy Chain (DNA)
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGTACCTCCGTCACAGTTAGCTCTGCTAG
CACAAAAGGCCCTAGCGTCTTTCCACTCGCCCCATCCTCCAAGAGCACCTCTGGGGGCACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAG
CTTGGGTACCCAGACCTATATCTGCAATGTCAATCATAAACCCAGTAACACAAAGGTAGACAAGAAAGTCGAA
CCCAAATCTTGCGACAAAACTCACACATGCCCTCCTTGCCCCGCCCCCGAAGCCGCCGGGGGACCTTCGGTTT
TTCTTTTTCCACCTAAACCGAAAGACACCCTGATGATCAGCAGGACACCAGAAGTGACATGTGTCGTCGTGGA
CGTATCCCATGAGGACCCGGAGGTGAAGTTCAACTGGTATGTTGATGGTGTGGAAGTGCATAATGCAAAAACT
AAGCCACGGGAGGAACAGTACAATAGCACATACAGAGTAGTTAGCGTTCTGACTGTCCTGCACCAAGATTGGC
TGAACGGGAAGGAATACAAGTGTAAGGTGAGCAACAAAGCCCTGCCCGCTCCAATTGAGAAAACAATTTCTAA
GGCCAAAGGACAGCCCCGGGAGCCACAGGTGTATACTCTGCCGCCTAGCAGGGAGGAAATGACCAAGAACCAG
GTGAGCCTGACCTGTCTGGTGAAAGGCTTCTACCCCAGCGATATCGCAGTTGAGTGGGAGAGCAATGGCCAAC
CCGAGAACAACTACAAGACTACGCCCCCCGTGCTTGATAGCGACGGATCATTTTTCCTGTACTCAAAACTGAC
CGTGGACAAAAGCAGATGGCAGCAGGGAAACGTTTTCAGTTGCTCAGTGATGTTCGAAGCGCTGCACAATCAT
TATACTCAGAAAAGCCTGAGCTTGAGCCCGGGAAAATGA
SEQ ID 212: Isatuximab IgG1 K2740 Heavy Chain (Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVQFNWYVDGVEVHNAKT
KPREEQYNSTYRVVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVFSCSVMHEALHNH
YTQKSLSLSPGK*
SEQ ID 213: Isatuximab IgG1 K2740 Heavy Chain (DNA)
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGTACCTCCGTCACAGTTAGCTCTGCTAG
CACAAAAGGCCCTAGCGTCTTTCCACTCGCCCCATCCTCCAAGAGCACCTCTGGGGGCACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAG
CTTGGGTACCCAGACCTATATCTGCAATGTCAATCATAAACCCAGTAACACAAAGGTAGACAAGAAAGTCGAA
CCCAAATCTTGCGACAAAACTCACACATGCCCTCCTTGCCCCGCCCCCGAACTCCTGGGGGGACCTTCGGTTT
TTCTTTTTCCACCTAAACCGAAAGACACCCTGATGATCAGCAGGACACCAGAAGTGACATGTGTCGTCGTGGA
CGTATCCCATGAGGACCCGGAGGTGCAGTTCAACTGGTATGTTGATGGTGTGGAAGTGCATAATGCAAAAACT
AAGCCACGGGAGGAACAGTACAATAGCACATACAGAGTAGTTAGCGTTCTGACTGTCCTGCACCAAGATTGGC
TGAACGGGAAGGAATACAAGTGTAAGGTGAGCAACAAAGCCCTGCCCGCTCCAATTGAGAAAACAATTTCTAA
GGCCAAAGGACAGCCCCGGGAGCCACAGGTGTATACTCTGCCGCCTAGCAGGGAGGAAATGACCAAGAACCAG
GTGAGCCTGACCTGTCTGGTGAAAGGCTTCTACCCCAGCGATATCGCAGTTGAGTGGGAGAGCAATGGCCAAC
CCGAGAACAACTACAAGACTACGCCCCCCGTGCTTGATAGCGACGGATCATTTTTCCTGTACTCAAAACTGAC
CGTGGACAAAAGCAGATGGCAGCAGGGAAACGTTTTCAGTTGCTCAGTGATGCACGAAGCGCTGCACAATCAT
TATACTCAGAAAAGCCTGAGCTTGAGCCCGGGAAAATGA
SEQ ID 214: Isatuximab IgG1 K2740 H429F Heavy Chain (Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSHEDPEVQFNWYVDGVEVHNAKT
KPREEQYNSTYRVVSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVFSCSVMFEALHNH
YTQKSLSLSPGK*
SEQ ID 215: Isatuximab IgG1 K2740 H429F Heavy Chain (DNA)
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGTACCTCCGTCACAGTTAGCTCTGCTAG
CACAAAAGGCCCTAGCGTCTTTCCACTCGCCCCATCCTCCAAGAGCACCTCTGGGGGCACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAG
CTTGGGTACCCAGACCTATATCTGCAATGTCAATCATAAACCCAGTAACACAAAGGTAGACAAGAAAGTCGAA
CCCAAATCTTGCGACAAAACTCACACATGCCCTCCTTGCCCCGCCCCCGAACTCCTGGGGGGACCTTCGGTTT
TTCTTTTTCCACCTAAACCGAAAGACACCCTGATGATCAGCAGGACACCAGAAGTGACATGTGTCGTCGTGGA
CGTATCCCATGAGGACCCGGAGGTGCAGTTCAACTGGTATGTTGATGGTGTGGAAGTGCATAATGCAAAAACT
AAGCCACGGGAGGAACAGTACAATAGCACATACAGAGTAGTTAGCGTTCTGACTGTCCTGCACCAAGATTGGC
TGAACGGGAAGGAATACAAGTGTAAGGTGAGCAACAAAGCCCTGCCCGCTCCAATTGAGAAAACAATTTCTAA
GGCCAAAGGACAGCCCCGGGAGCCACAGGTGTATACTCTGCCGCCTAGCAGGGAGGAAATGACCAAGAACCAG
GTGAGCCTGACCTGTCTGGTGAAAGGCTTCTACCCCAGCGATATCGCAGTTGAGTGGGAGAGCAATGGCCAAC
CCGAGAACAACTACAAGACTACGCCCCCCGTGCTTGATAGCGACGGATCATTTTTCCTGTACTCAAAACTGAC
CGTGGACAAAAGCAGATGGCAGCAGGGAAACGTTTTCAGTTGCTCAGTGATGTTCGAAGCGCTGCACAATCAT
TATACTCAGAAAAGCCTGAGCTTGAGCCCGGGAAAATGA
SEQ ID 216: Isatuximab IgG1 H268E A327D H429F Heavy Chain (Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVE
PKSCDKTHTCPPCPAPELLGGPSVFLFPPKPKDTLMISRTPEVTCVVVDVSEEDPEVKFNWYVDGVEVHNAKT
KPREEQYNSTYRVVSVLTVLHQDWLNGKEYKCKVSNKDLPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNVFSCSVMFEALHNH
YTQKSLSLSPGK*
SEQ ID 217: Isatuximab IgG1 H268E A327D H429F Heavy Chain (DNA)
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGTACCTCCGTCACAGTTAGCTCTGCTAG
CACAAAAGGCCCTAGCGTCTTTCCACTCGCCCCATCCTCCAAGAGCACCTCTGGGGGCACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAG
CTTGGGTACCCAGACCTATATCTGCAATGTCAATCATAAACCCAGTAACACAAAGGTAGACAAGAAAGTCGAA
CCCAAATCTTGCGACAAAACTCACACATGCCCTCCTTGCCCCGCCCCCGAACTCCTGGGGGGACCTTCGGTTT
TTCTTTTTCCACCTAAACCGAAAGACACCCTGATGATCAGCAGGACACCAGAAGTGACATGTGTCGTCGTGGA
CGTATCCGAGGAGGACCCGGAGGTGAAGTTCAACTGGTATGTTGATGGTGTGGAAGTGCATAATGCAAAAACT
AAGCCACGGGAGGAACAGTACAATAGCACATACAGAGTAGTTAGCGTTCTGACTGTCCTGCACCAAGATTGGC
TGAACGGGAAGGAATACAAGTGTAAGGTGAGCAACAAAGACCTGCCCGCTCCAATTGAGAAAACAATTTCTAA
GGCCAAAGGACAGCCCCGGGAGCCACAGGTGTATACTCTGCCGCCTAGCAGGGAGGAAATGACCAAGAACCAG
GTGAGCCTGACCTGTCTGGTGAAAGGCTTCTACCCCAGCGATATCGCAGTTGAGTGGGAGAGCAATGGCCAAC
CCGAGAACAACTACAAGACTACGCCCCCCGTGCTTGATAGCGACGGATCATTTTTCCTGTACTCAAAACTGAC
CGTGGACAAAAGCAGATGGCAGCAGGGAAACGTTTTCAGTTGCTCAGTGATGTTCGAAGCGCTGCACAATCAT
TATACTCAGAAAAGCCTGAGCTTGAGCCCGGGAAAATGA
SEQ ID 218: Isatuximab IgG2 LALA H429F Heavy Chain (Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPCSRSTSESTAALG
CLVKDYFPEPVTVSWNSGALTSGVHTFPAVLQSSGLYSLSSVVTVPSSNFGTQTYTCNVDHKPSNTKVDKTVE
RKCCVECPPCPAPEAAGGPSVFLFPPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKGQPREPQVYTLPPSREEMTKNQVSL
TCLVKGFYPSDIAVEWESNGQPENNYKTTPPMLDSDGSFFLYSKLTVDKSRWQQGNVFSCSVMFEALHNHYTQ
KSLSLSPG*
SEQ ID 219: Isatuximab IgG2 LALA H429F Heavy Chain (DNA)
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGTACCTCCGTCACAGTTAGCTCTGCTAG
CACAAAAGGCCCTAGCGTCTTTCCACTCGCCCCATGTTCAAGAAGCACCAGCGAGTCAACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAA
TTTTGGCACCCAGACCTATACATGCAATGTCGATCATAAACCCAGTAACACAAAGGTAGACAAGACAGTCGAA
AGAAAATGCTGTGTGGAGTGCCCTCCTTGCCCCGCCCCCGAAGCCGCCGGCGGCCCTTCGGTTTTTCTTTTTC
CACCTAAACCGAAAGACACCCTGATGATCAGCAGGACACCAGAAGTGACATGTGTCGTCGTGGACGTATCCCA
TGAAGACCCGGAGGTGCAATTCAACTGGTATGTTGATGGTGTGGAAGTGCATAATGCAAAAACTAAGCCACGG
GAGGAACAGTTCAATAGCACATTCAGAGTAGTTAGCGTTCTGACTGTCGTGCACCAAGATTGGCTGAACGGGA
AGGAATACAAGTGTAAGGTGAGCAACAAAGGTCTGCCCGCTCCAATTGAGAAAACAATTTCTAAGACCAAAGG
ACAGCCCCGGGAGCCACAGGTGTATACTCTGCCGCCTAGCAGGGAGGAAATGACCAAGAACCAGGTGAGCCTG
ACCTGTCTGGTGAAAGGCTTCTACCCCAGCGATATCGCAGTTGAGTGGGAGAGCAATGGCCAACCCGAGAACA
ACTACAAGACTACGCCCCCCATGCTTGATAGCGACGGATCATTTTTCCTGTACTCAAAACTGACCGTGGACAA
AAGCAGATGGCAGCAGGGAAACGTTTTCAGTTGCTCAGTGATGTTCGAAGCGCTGCACAATCATTATACTCAG
AAAAGCCTGAGCTTGAGCCCGGGATGA
SEQ ID 220: Isatuximab IgG2 0274K H429F Heavy Chain (Protein)
QVQLVQSGAEVAKPGTSVKLSCKASGYTFTDYWMQWVKQRPGQGLEWIGTIYPGDGDTGYAQKFQGKATLTAD
KSSKTVYMHLSSLASEDSAVYYCARGDYYGSNSLDYWGQGTSVTVSSASTKGPSVFPLAPCSRSTSESTAALG
CLVKDYFPEPVTVSWNSGALTSGVHTFPAVLQSSGLYSLSSVVTVPSSNFGTQTYTCNVDHKPSNTKVDKTVE
RKCCVECPPCPAPPVAGPSVFLFPPKPKDTLMISRTPEVTCVVVDVSHEDPEVKENWYVDGVEVHNAKTKPRE
EQFNSTFRVVSVLTVVHQDWLNGKEYKCKVSNKGLPAPIEKTISKTKGQPREPQVYTLPPSREEMTKNQVSLT
CLVKGFYPSDIAVEWESNGQPENNYKTTPPMLDSDGSFFLYSKLTVDKSRWQQGNVFSCSVMFEALHNHYTQK
SLSLSPG*
SEQ ID 221: Isatuximab IgG2 Q274K H429F Heavy Chain (DNA)
CAGGTTCAGCTGGTTCAGTCTGGCGCCGAGGTGGCCAAACCTGGCACATCTGTGAAGCTGAGCTGCAAGGCCA
GCGGCTACACCTTCACCGACTACTGGATGCAGTGGGTCAAGCAGAGGCCTGGACAGGGCCTCGAATGGATCGG
CACAATCTATCCTGGCGACGGCGATACAGGCTACGCCCAGAAGTTTCAGGGCAAAGCCACACTGACCGCCGAC
AAGAGCAGCAAGACCGTGTACATGCACCTGAGCAGCCTGGCCTCTGAGGATAGCGCCGTGTACTATTGCGCCA
GAGGCGACTACTACGGCAGCAACAGCCTGGATTATTGGGGCCAGGGTACCTCCGTCACAGTTAGCTCTGCTAG
CACAAAAGGCCCTAGCGTCTTTCCACTCGCCCCATGTTCAAGAAGCACCAGCGAGTCAACTGCGGCTTTGGGC
TGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAA
TTTTGGCACCCAGACCTATACATGCAATGTCGATCATAAACCCAGTAACACAAAGGTAGACAAGACAGTCGAA
AGAAAATGCTGTGTGGAGTGCCCTCCTTGCCCCGCCCCCCCAGTGGCCGGCCCTTCGGTTTTTCTTTTTCCAC
CTAAACCGAAAGACACCCTGATGATCAGCAGGACACCAGAAGTGACATGTGTCGTCGTGGACGTATCCCATGA
AGACCCGGAGGTGAAGTTCAACTGGTATGTTGATGGTGTGGAAGTGCATAATGCAAAAACTAAGCCACGGGAG
GAACAGTTCAATAGCACATTCAGAGTAGTTAGCGTTCTGACTGTCGTGCACCAAGATTGGCTGAACGGGAAGG
AATACAAGTGTAAGGTGAGCAACAAAGGTCTGCCCGCTCCAATTGAGAAAACAATTTCTAAGACCAAAGGACA
GCCCCGGGAGCCACAGGTGTATACTCTGCCGCCTAGCAGGGAGGAAATGACCAAGAACCAGGTGAGCCTGACC
TGTCTGGTGAAAGGCTTCTACCCCAGCGATATCGCAGTTGAGTGGGAGAGCAATGGCCAACCCGAGAACAACT
ACAAGACTACGCCCCCCATGCTTGATAGCGACGGATCATTTTTCCTGTACTCAAAACTGACCGTGGACAAAAG
CAGATGGCAGCAGGGAAACGTTTTCAGTTGCTCAGTGATGTTCGAAGCGCTGCACAATCATTATACTCAGAAA
AGCCTGAGCTTGAGCCCGGGATGA
SEQ ID 222: Rituximab IgG1 H429F Heavy Chain (Protein)
QVQLQQPGAELVKPGASVKMSCKASGYTFTSYNMHWVKQTPGRGLEWIGAIYPGNGDTSYNQKFKGKATLTAD
KSSSTAYMQLSSLTSEDSAVYYCARSTYYGGDWYFNVWGAGTTVTVSAASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVVDVSHEDPEVKENWYVDGVEVHNAK
TKPREEQYNSTYRVVSVLTVLHQDWLNGKEYKCKVSNKALPAPIEKTISKAKGQPREPQVYTLPPSRDELTKN
QVSLTCLVKGFYPSDIAVEWESNGQPENNYKTTPPVLDSDGSFFLYSKLTVDKSRWQQGNVFSCSVMFEALHN
HYTQKSLSLSPGK
SEQ ID 223: Rituximab IgG1 H429F Heavy Chain (DNA)
CAGGTTCAGCTGCAACAGCCTGGCGCCGAGCTTGTGAAACCTGGCGCCTCTGTGAAGATGAGCTGCAAGGCCA
GCGGCTACACCTTCACCAGCTACAACATGCACTGGGTCAAGCAGACCCCTGGCAGAGGCCTGGAATGGATCGG
AGCCATCTATCCCGGCAACGGCGACACCTCCTACAACCAGAAGTTCAAGGGCAAAGCCACACTGACCGCCGAC
AAGAGCAGCAGCACAGCCTACATGCAGCTGAGCAGCCTGACCAGCGAAGATAGCGCCGTGTACTACTGCGCCA
GAAGCACCTATTACGGCGGCGACTGGTACTTCAACGTGTGGGGAGCTGGCACCACCGTGACAGTTTCTGCCGC
TAGCACAAAAGGCCCTAGCGTCTTTCCACTCGCCCCATCCTCCAAGAGCACCTCTGGGGGCACTGCGGCTTTG
GGCTGCTTGGTGAAGGATTACTTCCCAGAGCCTGTCACTGTCAGCTGGAATAGCGGGGCTCTGACCAGTGGAG
TGCACACCTTTCCCGCCGTGTTACAGAGCAGCGGCCTCTATAGCCTGAGCAGTGTGGTAACTGTGCCCTCGAG
CAGCTTGGGTACCCAGACCTATATCTGCAATGTCAATCATAAACCCAGTAACACAAAGGTAGACAAGAAAGTC
GAACCCAAATCTTGCGACAAAACTCACACATGCCCTCCTTGCCCCGCCCCCGAACTCCTGGGGGGACCTTCGG
TTTTTCTTTTTCCACCTAAACCGAAAGACACCCTGATGATCAGCAGGACACCAGAAGTGACATGTGTCGTCGT
GGACGTATCCCATGAGGACCCGGAGGTGAAGTTCAACTGGTATGTTGATGGTGTGGAAGTGCATAATGCAAAA
ACTAAGCCACGGGAGGAACAGTACAATAGCACATACAGAGTAGTTAGCGTTCTGACTGTCCTGCACCAAGATT
GGCTGAACGGGAAGGAATACAAGTGTAAGGTGAGCAACAAAGCCCTGCCCGCTCCAATTGAGAAAACAATTTC
TAAGGCCAAAGGACAGCCCCGGGAGCCACAGGTGTATACTCTGCCGCCTAGCAGGGATGAACTGACCAAGAAC
CAGGTGAGCCTGACCTGTCTGGTGAAAGGCTTCTACCCCAGCGATATCGCAGTTGAGTGGGAGAGCAATGGCC
AACCCGAGAACAACTACAAGACTACGCCCCCCGTGCTTGATAGCGACGGATCATTTTTCCTGTACTCAAAACT
GACCGTGGACAAAAGCAGATGGCAGCAGGGAAACGTTTTCAGTTGCTCAGTGATGttCGAgGCGCTGCACAAT
CATTATACTCAGAAAAGCCTGAGCTTGAGCCCGGGAAAA
SEQ ID 224: S2P6 IgG2 WT (Protein)
EVQLVQSGAEVKKPGASVKVSCKASGYTFTSQYMHWVRQAPGQGLEWIGIINPSGVHTSYAQKFQGRVTLTRD
TSTSTLYMELSSLRSEDTAVYYCARGSPKGAFDYWGQGTLVTVSSASTKGPSVFPLAPCSRSTSESTAALGCL
VKDYFPEPVTVSWNSGALTSGVHTFPAVLQSSGLYSLSSVVTVPSSNFGTQTYTCNVDHKPSNTKVDKTVERK
CCVECPPCPAPPVAGPSVFLFPPKPKDTLMISRTPEVTCVVVDVSHEDPEVQFNWYVDGVEVHNAKTKPREEQ
FNSTFRVVSVLTVVHQDWLNGKEYKCKVSNKGLPAPIEKTISKTKGQPREPQVYTLPPSREEMTKNQVSLTCL
VKGFYPSDIAVEWESNGQPENNYKTTPPMLDSDGSFFLYSKLTVDKSRWQQGNVFSCSVMHEALHNHYTQKSL
SLSPG
SEQ ID 225: S2P6 IgG2 WT (DNA)
GAAGTTCAGCTGGTTCAGTCTGGCGCCGAAGTGAAGAAACCTGGCGCCTCTGTGAAGGTGTCCTGCAAGGCCA
GCGGCTACACCTTTACCAGCCAGTACATGCACTGGGTCCGACAGGCTCCAGGACAGGGACTCGAGTGGATCGG
CATCATCAATCCTAGCGGCGTGCACACCAGCTACGCCCAGAAATTCCAGGGCAGAGTGACCCTGACCAGAGAC
ACCAGCACCAGCACACTGTACATGGAACTGAGCAGCCTGCGGAGCGAGGATACCGCCGTGTACTATTGTGCCA
GAGGCTCTCCTAAGGGCGCCTTCGATTATTGGGGCCAGGGTACCCTGGTCACCGTGTCCTCTGCTAGCACAAA
AGGCCCTAGCGTCTTTCCACTCGCCCCATGTTCAAGAAGCACCAGCGAGTCAACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAATTTTGG
CACCCAGACCTATACATGCAATGTCGATCATAAACCCAGTAACACAAAGGTAGACAAGACAGTCGAAAGAAAA
TGCTGTGTGGAGTGCCCTCCTTGCCCCGCCCCCCCAGTGGCCGGGCCTTCGGTTTTTCTTTTTCCACCTAAAC
CGAAAGACACCCTGATGATCAGCAGGACACCAGAAGTGACATGTGTCGTCGTGGACGTATCCCATGAAGACCC
GGAGGTGCAATTCAACTGGTATGTTGATGGTGTGGAAGTGCATAATGCAAAAACTAAGCCACGGGAGGAACAG
TTCAATAGCACATTCAGAGTAGTTAGCGTTCTGACTGTCGTGCACCAAGATTGGCTGAACGGGAAGGAATACA
AGTGTAAGGTGAGCAACAAAGGTCTGCCCGCTCCAATTGAGAAAACAATTTCTAAGACCAAAGGACAGCCCCG
GGAGCCACAGGTGTATACTCTGCCGCCTAGCAGGGAGGAAATGACCAAGAACCAGGTGAGCCTGACCTGTCTG
GTGAAAGGCTTCTACCCCAGCGATATCGCAGTTGAGTGGGAGAGCAATGGCCAACCCGAGAACAACTACAAGA
CTACGCCCCCCATGCTTGATAGCGACGGATCATTTTTCCTGTACTCAAAACTGACCGTGGACAAAAGCAGATG
GCAGCAGGGAAACGTTTTCAGTTGCTCAGTGATGCACGAAGCGCTGCACAATCATTATACTCAGAAAAGCCTG
AGCTTGAGCCCGGGA
SEQ ID 226: S2P6 IgG2 H429F (Protein)
EVQLVQSGAEVKKPGASVKVSCKASGYTFTSQYMHWVRQAPGQGLEWIGIINPSGVHTSYAQKFQGRVTLTRD
TSTSTLYMELSSLRSEDTAVYYCARGSPKGAFDYWGQGTLVTVSSASTKGPSVFPLAPCSRSTSESTAALGCL
VKDYFPEPVTVSWNSGALTSGVHTFPAVLQSSGLYSLSSVVTVPSSNFGTQTYTCNVDHKPSNTKVDKTVERK
CCVECPPCPAPPVAGPSVFLFPPKPKDTLMISRTPEVTCVVVDVSHEDPEVQFNWYVDGVEVHNAKTKPREEQ
FNSTFRVVSVLTVVHQDWLNGKEYKCKVSNKGLPAPIEKTISKTKGQPREPQVYTLPPSREEMTKNQVSLTCL
VKGFYPSDIAVEWESNGQPENNYKTTPPMLDSDGSFFLYSKLTVDKSRWQQGNVFSCSVMFEALHNHYTQKSL
SLSPG
SEQ ID 227: S2P6 IgG2 H429F (DNA)
GAAGTTCAGCTGGTTCAGTCTGGCGCCGAAGTGAAGAAACCTGGCGCCTCTGTGAAGGTGTCCTGCAAGGCCA
GCGGCTACACCTTTACCAGCCAGTACATGCACTGGGTCCGACAGGCTCCAGGACAGGGACTCGAGTGGATCGG
CATCATCAATCCTAGCGGCGTGCACACCAGCTACGCCCAGAAATTCCAGGGCAGAGTGACCCTGACCAGAGAC
ACCAGCACCAGCACACTGTACATGGAACTGAGCAGCCTGCGGAGCGAGGATACCGCCGTGTACTATTGTGCCA
GAGGCTCTCCTAAGGGCGCCTTCGATTATTGGGGCCAGGGTACCCTGGTCACCGTGTCCTCTGCTAGCACAAA
AGGCCCTAGCGTCTTTCCACTCGCCCCATGTTCAAGAAGCACCAGCGAGTCAACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAATTTTGG
CACCCAGACCTATACATGCAATGTCGATCATAAACCCAGTAACACAAAGGTAGACAAGACAGTCGAAAGAAAA
TGCTGTGTGGAGTGCCCTCCTTGCCCCGCCCCCCCAGTGGCCGGGCCTTCGGTTTTTCTTTTTCCACCTAAAC
CGAAAGACACCCTGATGATCAGCAGGACACCAGAAGTGACATGTGTCGTCGTGGACGTATCCCATGAAGACCC
GGAGGTGCAATTCAACTGGTATGTTGATGGTGTGGAAGTGCATAATGCAAAAACTAAGCCACGGGAGGAACAG
TTCAATAGCACATTCAGAGTAGTTAGCGTTCTGACTGTCGTGCACCAAGATTGGCTGAACGGGAAGGAATACA
AGTGTAAGGTGAGCAACAAAGGTCTGCCCGCTCCAATTGAGAAAACAATTTCTAAGACCAAAGGACAGCCCCG
GGAGCCACAGGTGTATACTCTGCCGCCTAGCAGGGAGGAAATGACCAAGAACCAGGTGAGCCTGACCTGTCTG
GTGAAAGGCTTCTACCCCAGCGATATCGCAGTTGAGTGGGAGAGCAATGGCCAACCCGAGAACAACTACAAGA
CTACGCCCCCCATGCTTGATAGCGACGGATCATTTTTCCTGTACTCAAAACTGACCGTGGACAAAAGCAGATG
GCAGCAGGGAAACGTTTTCAGTTGCTCAGTGATGTTCGAAGCGCTGCACAATCATTATACTCAGAAAAGCCTG
AGCTTGAGCCCGGGA
SEQ ID 228: S2P6 IgG2 C131S C220S FLGG (Protein)
EVQLVQSGAEVKKPGASVKVSCKASGYTFTSQYMHWVRQAPGQGLEWIGIINPSGVHTSYAQKFQGRVTLTRD
TSTSTLYMELSSLRSEDTAVYYCARGSPKGAFDYWGQGTLVTVSSASTKGPSVFPLAPSSRSTSESTAALGCL
VKDYFPEPVTVSWNSGALTSGVHTFPAVLQSSGLYSLSSVVTVPSSNFGTQTYTCNVDHKPSNTKVDKTVERK
CSVECPPCPAPEFLGGPSVFLFPPKPKDTLMISRTPEVTCVVVDVSHEDPEVQFNWYVDGVEVHNAKTKPREE
QFNSTFRVVSVLTVVHQDWLNGKEYKCKVSNKGLPAPIEKTISKTKGQPREPQVYTLPPSREEMTKNQVSLTC
LVKGFYPSDIAVEWESNGQPENNYKTTPPMLDSDGSFFLYSKLTVDKSRWQQGNVFSCSVMHEALHNHYTQKS
LSLSPG
SEQ ID 229: S2P6 IgG2 C131S C220S FLGG (DNA)
GAAGTTCAGCTGGTTCAGTCTGGCGCCGAAGTGAAGAAACCTGGCGCCTCTGTGAAGGTGTCCTGCAAGGCCA
GCGGCTACACCTTTACCAGCCAGTACATGCACTGGGTCCGACAGGCTCCAGGACAGGGACTCGAGTGGATCGG
CATCATCAATCCTAGCGGCGTGCACACCAGCTACGCCCAGAAATTCCAGGGCAGAGTGACCCTGACCAGAGAC
ACCAGCACCAGCACACTGTACATGGAACTGAGCAGCCTGCGGAGCGAGGATACCGCCGTGTACTATTGTGCCA
GAGGCTCTCCTAAGGGCGCCTTCGATTATTGGGGCCAGGGTACCCTGGTCACCGTGTCCTCTGCTAGCACAAA
AGGCCCTAGCGTCTTTCCACTCGCCCCAAGCTCAAGAAGCACCAGCGAGTCAACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAATTTTGG
CACCCAGACCTATACATGCAATGTCGATCATAAACCCAGTAACACAAAGGTAGACAAGACAGTCGAAAGAAAA
TGCAGCGTGGAGTGCCCTCCTTGCCCCGCCCCCGAATTCCTGGGCGGCCCTTCGGTTTTTCTTTTTCCACCTA
AACCGAAAGACACCCTGATGATCAGCAGGACACCAGAAGTGACATGTGTCGTCGTGGACGTATCCCATGAAGA
CCCGGAGGTGCAATTCAACTGGTATGTTGATGGTGTGGAAGTGCATAATGCAAAAACTAAGCCACGGGAGGAA
CAGTTCAATAGCACATTCAGAGTAGTTAGCGTTCTGACTGTCGTGCACCAAGATTGGCTGAACGGGAAGGAAT
ACAAGTGTAAGGTGAGCAACAAAGGTCTGCCCGCTCCAATTGAGAAAACAATTTCTAAGACCAAAGGACAGCC
CCGGGAGCCACAGGTGTATACTCTGCCGCCTAGCAGGGAGGAAATGACCAAGAACCAGGTGAGCCTGACCTGT
CTGGTGAAAGGCTTCTACCCCAGCGATATCGCAGTTGAGTGGGAGAGCAATGGCCAACCCGAGAACAACTACA
AGACTACGCCCCCCATGCTTGATAGCGACGGATCATTTTTCCTGTACTCAAAACTGACCGTGGACAAAAGCAG
ATGGCAGCAGGGAAACGTTTTCAGTTGCTCAGTGATGCACGAAGCGCTGCACAATCATTATACTCAGAAAAGC
CTGAGCTTGAGCCCGGGA
SEQ ID 230: S2P6 IgG2 C131S C220S FLGG H429F (Protein)
EVQLVQSGAEVKKPGASVKVSCKASGYTFTSQYMHWVRQAPGQGLEWIGIINPSGVHTSYAQKFQGRVTLTRD
TSTSTLYMELSSLRSEDTAVYYCARGSPKGAFDYWGQGTLVTVSSASTKGPSVFPLAPSSRSTSESTAALGCL
VKDYFPEPVTVSWNSGALTSGVHTFPAVLQSSGLYSLSSVVTVPSSNFGTQTYTCNVDHKPSNTKVDKTVERK
CSVECPPCPAPEFLGGPSVFLFPPKPKDTLMISRTPEVTCVVVDVSHEDPEVQFNWYVDGVEVHNAKTKPREE
QFNSTFRVVSVLTVVHQDWLNGKEYKCKVSNKGLPAPIEKTISKTKGQPREPQVYTLPPSREEMTKNQVSLTC
LVKGFYPSDIAVEWESNGQPENNYKTTPPMLDSDGSFFLYSKLTVDKSRWQQGNVFSCSVMFEALHNHYTQKS
LSLSPG
SEQ ID 231: S2P6 IgG2 C131S C220S FLGG H429F (DNA)
GAAGTTCAGCTGGTTCAGTCTGGCGCCGAAGTGAAGAAACCTGGCGCCTCTGTGAAGGTGTCCTGCAAGGCCA
GCGGCTACACCTTTACCAGCCAGTACATGCACTGGGTCCGACAGGCTCCAGGACAGGGACTCGAGTGGATCGG
CATCATCAATCCTAGCGGCGTGCACACCAGCTACGCCCAGAAATTCCAGGGCAGAGTGACCCTGACCAGAGAC
ACCAGCACCAGCACACTGTACATGGAACTGAGCAGCCTGCGGAGCGAGGATACCGCCGTGTACTATTGTGCCA
GAGGCTCTCCTAAGGGCGCCTTCGATTATTGGGGCCAGGGTACCCTGGTCACCGTGTCCTCTGCTAGCACAAA
AGGCCCTAGCGTCTTTCCACTCGCCCCAAGCTCAAGAAGCACCAGCGAGTCAACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAATTTTGG
CACCCAGACCTATACATGCAATGTCGATCATAAACCCAGTAACACAAAGGTAGACAAGACAGTCGAAAGAAAA
TGCAGCGTGGAGTGCCCTCCTTGCCCCGCCCCCGAATTCCTGGGCGGCCCTTCGGTTTTTCTTTTTCCACCTA
AACCGAAAGACACCCTGATGATCAGCAGGACACCAGAAGTGACATGTGTCGTCGTGGACGTATCCCATGAAGA
CCCGGAGGTGCAATTCAACTGGTATGTTGATGGTGTGGAAGTGCATAATGCAAAAACTAAGCCACGGGAGGAA
CAGTTCAATAGCACATTCAGAGTAGTTAGCGTTCTGACTGTCGTGCACCAAGATTGGCTGAACGGGAAGGAAT
ACAAGTGTAAGGTGAGCAACAAAGGTCTGCCCGCTCCAATTGAGAAAACAATTTCTAAGACCAAAGGACAGCC
CCGGGAGCCACAGGTGTATACTCTGCCGCCTAGCAGGGAGGAAATGACCAAGAACCAGGTGAGCCTGACCTGT
CTGGTGAAAGGCTTCTACCCCAGCGATATCGCAGTTGAGTGGGAGAGCAATGGCCAACCCGAGAACAACTACA
AGACTACGCCCCCCATGCTTGATAGCGACGGATCATTTTTCCTGTACTCAAAACTGACCGTGGACAAAAGCAG
ATGGCAGCAGGGAAACGTTTTCAGTTGCTCAGTGATGTTCGAAGCGCTGCACAATCATTATACTCAGAAAAGC
CTGAGCTTGAGCCCGGGA
SEQ ID 232: S2P6 IgG2 C131S C219S FLGG (Protein)
EVQLVQSGAEVKKPGASVKVSCKASGYTFTSQYMHWVRQAPGQGLEWIGIINPSGVHTSYAQKFQGRVTLTRD
TSTSTLYMELSSLRSEDTAVYYCARGSPKGAFDYWGQGTLVTVSSASTKGPSVFPLAPSSRSTSESTAALGCL
VKDYFPEPVTVSWNSGALTSGVHTFPAVLQSSGLYSLSSVVTVPSSNFGTQTYTCNVDHKPSNTKVDKTVERK
SCVECPPCPAPEFLGGPSVFLFPPKPKDTLMISRTPEVTCVVVDVSHEDPEVQFNWYVDGVEVHNAKTKPREE
QFNSTFRVVSVLTVVHQDWLNGKEYKCKVSNKGLPAPIEKTISKTKGQPREPQVYTLPPSREEMTKNQVSLTC
LVKGFYPSDIAVEWESNGQPENNYKTTPPMLDSDGSFFLYSKLTVDKSRWQQGNVFSCSVMHEALHNHYTQKS
LSLSPG
SEQ ID 233: S2P6 IgG2 C131S C219S FLGG (DNA)
GAAGTTCAGCTGGTTCAGTCTGGCGCCGAAGTGAAGAAACCTGGCGCCTCTGTGAAGGTGTCCTGCAAGGCCA
GCGGCTACACCTTTACCAGCCAGTACATGCACTGGGTCCGACAGGCTCCAGGACAGGGACTCGAGTGGATCGG
CATCATCAATCCTAGCGGCGTGCACACCAGCTACGCCCAGAAATTCCAGGGCAGAGTGACCCTGACCAGAGAC
ACCAGCACCAGCACACTGTACATGGAACTGAGCAGCCTGCGGAGCGAGGATACCGCCGTGTACTATTGTGCCA
GAGGCTCTCCTAAGGGCGCCTTCGATTATTGGGGCCAGGGTACCCTGGTCACCGTGTCCTCTGCTAGCACAAA
AGGCCCTAGCGTCTTTCCACTCGCCCCAAGCTCAAGAAGCACCAGCGAGTCAACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAATTTTGG
CACCCAGACCTATACATGCAATGTCGATCATAAACCCAGTAACACAAAGGTAGACAAGACAGTCGAAAGAAAA
AGCTGCGTGGAGTGCCCTCCTTGCCCCGCCCCCGAATTCCTGGGCGGCCCTTCGGTTTTTCTTTTTCCACCTA
AACCGAAAGACACCCTGATGATCAGCAGGACACCAGAAGTGACATGTGTCGTCGTGGACGTATCCCATGAAGA
CCCGGAGGTGCAATTCAACTGGTATGTTGATGGTGTGGAAGTGCATAATGCAAAAACTAAGCCACGGGAGGAA
CAGTTCAATAGCACATTCAGAGTAGTTAGCGTTCTGACTGTCGTGCACCAAGATTGGCTGAACGGGAAGGAAT
ACAAGTGTAAGGTGAGCAACAAAGGTCTGCCCGCTCCAATTGAGAAAACAATTTCTAAGACCAAAGGACAGCC
CCGGGAGCCACAGGTGTATACTCTGCCGCCTAGCAGGGAGGAAATGACCAAGAACCAGGTGAGCCTGACCTGT
CTGGTGAAAGGCTTCTACCCCAGCGATATCGCAGTTGAGTGGGAGAGCAATGGCCAACCCGAGAACAACTACA
AGACTACGCCCCCCATGCTTGATAGCGACGGATCATTTTTCCTGTACTCAAAACTGACCGTGGACAAAAGCAG
ATGGCAGCAGGGAAACGTTTTCAGTTGCTCAGTGATGCACGAAGCGCTGCACAATCATTATACTCAGAAAAGC
CTGAGCTTGAGCCCGGGA
SEQ ID 234: S2P6 IgG2 C131S C219S FLGG H429F (Protein)
EVQLVQSGAEVKKPGASVKVSCKASGYTFTSQYMHWVRQAPGQGLEWIGIINPSGVHTSYAQKFQGRVTLTRD
TSTSTLYMELSSLRSEDTAVYYCARGSPKGAFDYWGQGTLVTVSSASTKGPSVFPLAPSSRSTSESTAALGCL
VKDYFPEPVTVSWNSGALTSGVHTFPAVLQSSGLYSLSSVVTVPSSNFGTQTYTCNVDHKPSNTKVDKTVERK
SCVECPPCPAPEFLGGPSVFLFPPKPKDTLMISRTPEVTCVVVDVSHEDPEVQFNWYVDGVEVHNAKTKPREE
QFNSTFRVVSVLTVVHQDWLNGKEYKCKVSNKGLPAPIEKTISKTKGQPREPQVYTLPPSREEMTKNQVSLTC
LVKGFYPSDIAVEWESNGQPENNYKTTPPMLDSDGSFFLYSKLTVDKSRWQQGNVFSCSVMFEALHNHYTQKS
LSLSPG
SEQ ID 235: S2P6 IgG2 C131S C219S FLGG H429F (DNA)
GAAGTTCAGCTGGTTCAGTCTGGCGCCGAAGTGAAGAAACCTGGCGCCTCTGTGAAGGTGTCCTGCAAGGCCA
GCGGCTACACCTTTACCAGCCAGTACATGCACTGGGTCCGACAGGCTCCAGGACAGGGACTCGAGTGGATCGG
CATCATCAATCCTAGCGGCGTGCACACCAGCTACGCCCAGAAATTCCAGGGCAGAGTGACCCTGACCAGAGAC
ACCAGCACCAGCACACTGTACATGGAACTGAGCAGCCTGCGGAGCGAGGATACCGCCGTGTACTATTGTGCCA
GAGGCTCTCCTAAGGGCGCCTTCGATTATTGGGGCCAGGGTACCCTGGTCACCGTGTCCTCTGCTAGCACAAA
AGGCCCTAGCGTCTTTCCACTCGCCCCAAGCTCAAGAAGCACCAGCGAGTCAACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAATTTTGG
CACCCAGACCTATACATGCAATGTCGATCATAAACCCAGTAACACAAAGGTAGACAAGACAGTCGAAAGAAAA
AGCTGCGTGGAGTGCCCTCCTTGCCCCGCCCCCGAATTCCTGGGCGGCCCTTCGGTTTTTCTTTTTCCACCTA
AACCGAAAGACACCCTGATGATCAGCAGGACACCAGAAGTGACATGTGTCGTCGTGGACGTATCCCATGAAGA
CCCGGAGGTGCAATTCAACTGGTATGTTGATGGTGTGGAAGTGCATAATGCAAAAACTAAGCCACGGGAGGAA
CAGTTCAATAGCACATTCAGAGTAGTTAGCGTTCTGACTGTCGTGCACCAAGATTGGCTGAACGGGAAGGAAT
ACAAGTGTAAGGTGAGCAACAAAGGTCTGCCCGCTCCAATTGAGAAAACAATTTCTAAGACCAAAGGACAGCC
CCGGGAGCCACAGGTGTATACTCTGCCGCCTAGCAGGGAGGAAATGACCAAGAACCAGGTGAGCCTGACCTGT
CTGGTGAAAGGCTTCTACCCCAGCGATATCGCAGTTGAGTGGGAGAGCAATGGCCAACCCGAGAACAACTACA
AGACTACGCCCCCCATGCTTGATAGCGACGGATCATTTTTCCTGTACTCAAAACTGACCGTGGACAAAAGCAG
ATGGCAGCAGGGAAACGTTTTCAGTTGCTCAGTGATGTTCGAAGCGCTGCACAATCATTATACTCAGAAAAGC
CTGAGCTTGAGCCCGGGA
SEQ ID 236: S2P6 IgG2 C131S uH1 FLGG (Protein)
EVQLVQSGAEVKKPGASVKVSCKASGYTFTSQYMHWVRQAPGQGLEWIGIINPSGVHTSYAQKFQGRVTLTRD
TSTSTLYMELSSLRSEDTAVYYCARGSPKGAFDYWGQGTLVTVSSASTKGPSVFPLAPSSRSTSESTAALGCL
VKDYFPEPVTVSWNSGALTSGVHTFPAVLQSSGLYSLSSVVTVPSSNFGTQTYTCNVDHKPSNTKVDKTVEPK
SCDKTHTCPPCPAPEFLGGPSVFLFPPKPKDTLMISRTPEVTCVVVDVSHEDPEVQFNWYVDGVEVHNAKTKP
REEQFNSTFRVVSVLTVVHQDWLNGKEYKCKVSNKGLPAPIEKTISKTKGQPREPQVYTLPPSREEMTKNQVS
LTCLVKGFYPSDIAVEWESNGQPENNYKTTPPMLDSDGSFFLYSKLTVDKSRWQQGNVFSCSVMHEALHNHYT
QKSLSLSPG
SEQ ID 237: S2P6 IgG2 C131S uH1 FLGG (DNA)
GAAGTTCAGCTGGTTCAGTCTGGCGCCGAAGTGAAGAAACCTGGCGCCTCTGTGAAGGTGTCCTGCAAGGCCA
GCGGCTACACCTTTACCAGCCAGTACATGCACTGGGTCCGACAGGCTCCAGGACAGGGACTCGAGTGGATCGG
CATCATCAATCCTAGCGGCGTGCACACCAGCTACGCCCAGAAATTCCAGGGCAGAGTGACCCTGACCAGAGAC
ACCAGCACCAGCACACTGTACATGGAACTGAGCAGCCTGCGGAGCGAGGATACCGCCGTGTACTATTGTGCCA
GAGGCTCTCCTAAGGGCGCCTTCGATTATTGGGGCCAGGGTACCCTGGTCACCGTGTCCTCTGCTAGCACAAA
AGGCCCTAGCGTCTTTCCACTCGCCCCAAGCTCAAGAAGCACCAGCGAGTCAACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAATTTTGG
CACCCAGACCTATACATGCAATGTCGATCATAAACCCAGTAACACAAAGGTAGACAAGACAGTCGAACCCAAG
AGCTGCGACAAGACCCACACATGTCCACCATGCCCCGCCCCCGAATTCCTGGGCGGCCCTTCGGTTTTTCTTT
TTCCACCTAAACCGAAAGACACCCTGATGATCAGCAGGACACCAGAAGTGACATGTGTCGTCGTGGACGTATC
CCATGAAGACCCGGAGGTGCAATTCAACTGGTATGTTGATGGTGTGGAAGTGCATAATGCAAAAACTAAGCCA
CGGGAGGAACAGTTCAATAGCACATTCAGAGTAGTTAGCGTTCTGACTGTCGTGCACCAAGATTGGCTGAACG
GGAAGGAATACAAGTGTAAGGTGAGCAACAAAGGTCTGCCCGCTCCAATTGAGAAAACAATTTCTAAGACCAA
AGGACAGCCCCGGGAGCCACAGGTGTATACTCTGCCGCCTAGCAGGGAGGAAATGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCATGCTTGATAGCGACGGATCATTTTTCCTGTACTCAAAACTGACCGTGGA
CAAAAGCAGATGGCAGCAGGGAAACGTTTTCAGTTGCTCAGTGATGCACGAAGCGCTGCACAATCATTATACT
CAGAAAAGCCTGAGCTTGAGCCCGGGA
SEQ ID 238: S2P6 IgG2 C131S uH1 FLGG H429F (Protein)
EVQLVQSGAEVKKPGASVKVSCKASGYTFTSQYMHWVRQAPGQGLEWIGIINPSGVHTSYAQKFQGRVTLTRD
TSTSTLYMELSSLRSEDTAVYYCARGSPKGAFDYWGQGTLVTVSSASTKGPSVFPLAPSSRSTSESTAALGCL
VKDYFPEPVTVSWNSGALTSGVHTFPAVLQSSGLYSLSSVVTVPSSNFGTQTYTCNVDHKPSNTKVDKTVEPK
SCDKTHTCPPCPAPEFLGGPSVFLFPPKPKDTLMISRTPEVTCVVVDVSHEDPEVQFNWYVDGVEVHNAKTKP
REEQFNSTFRVVSVLTVVHQDWLNGKEYKCKVSNKGLPAPIEKTISKTKGQPREPQVYTLPPSREEMTKNQVS
LTCLVKGFYPSDIAVEWESNGQPENNYKTTPPMLDSDGSFFLYSKLTVDKSRWQQGNVFSCSVMFEALHNHYT
QKSLSLSPG
SEQ ID 239: S2P6 IgG2 C131S uH1 FLGG H429F (DNA)
GAAGTTCAGCTGGTTCAGTCTGGCGCCGAAGTGAAGAAACCTGGCGCCTCTGTGAAGGTGTCCTGCAAGGCCA
GCGGCTACACCTTTACCAGCCAGTACATGCACTGGGTCCGACAGGCTCCAGGACAGGGACTCGAGTGGATCGG
CATCATCAATCCTAGCGGCGTGCACACCAGCTACGCCCAGAAATTCCAGGGCAGAGTGACCCTGACCAGAGAC
ACCAGCACCAGCACACTGTACATGGAACTGAGCAGCCTGCGGAGCGAGGATACCGCCGTGTACTATTGTGCCA
GAGGCTCTCCTAAGGGCGCCTTCGATTATTGGGGCCAGGGTACCCTGGTCACCGTGTCCTCTGCTAGCACAAA
AGGCCCTAGCGTCTTTCCACTCGCCCCAAGCTCAAGAAGCACCAGCGAGTCAACTGCGGCTTTGGGCTGCTTG
GTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAATTTTGG
CACCCAGACCTATACATGCAATGTCGATCATAAACCCAGTAACACAAAGGTAGACAAGACAGTCGAACCCAAG
AGCTGCGACAAGACCCACACATGTCCACCATGCCCCGCCCCCGAATTCCTGGGCGGCCCTTCGGTTTTTCTTT
TTCCACCTAAACCGAAAGACACCCTGATGATCAGCAGGACACCAGAAGTGACATGTGTCGTCGTGGACGTATC
CCATGAAGACCCGGAGGTGCAATTCAACTGGTATGTTGATGGTGTGGAAGTGCATAATGCAAAAACTAAGCCA
CGGGAGGAACAGTTCAATAGCACATTCAGAGTAGTTAGCGTTCTGACTGTCGTGCACCAAGATTGGCTGAACG
GGAAGGAATACAAGTGTAAGGTGAGCAACAAAGGTCTGCCCGCTCCAATTGAGAAAACAATTTCTAAGACCAA
AGGACAGCCCCGGGAGCCACAGGTGTATACTCTGCCGCCTAGCAGGGAGGAAATGACCAAGAACCAGGTGAGC
CTGACCTGTCTGGTGAAAGGCTTCTACCCCAGCGATATCGCAGTTGAGTGGGAGAGCAATGGCCAACCCGAGA
ACAACTACAAGACTACGCCCCCCATGCTTGATAGCGACGGATCATTTTTCCTGTACTCAAAACTGACCGTGGA
CAAAAGCAGATGGCAGCAGGGAAACGTTTTCAGTTGCTCAGTGATGTTCGAAGCGCTGCACAATCATTATACT
CAGAAAAGCCTGAGCTTGAGCCCGGGA
SEQ ID NO: 240 Human IgG1 HC (J00228) upper hinge residues
PKSCDKTHT
SEQ ID NO: 241 Human IgG2 HC (J00230) upper hinge residues
RKCCVE
SEQ ID NO: 242 Human IgG1 HC (J00228) upper hinge residues
EPKSCDKTHT
SEQ ID NO: 243 Human IgG2 HC (J00230) upper hinge residues
ERKCCVE
SEQ ID NO: 244 Human IgG3 HC (M12958) H1 upper hinge residues
ELKTPLGDTTHT
SEQ ID NO: 245 Human IgG4 HC (K01316) upper hinge residues
ESKYGPP
SEQ ID NO: 246 Human IgG1 HC (J00228) and IgG2 HC (J00230) core hinge
CPPCP
SEQ ID NO: 247 Human IgG3 HC (M12958) core hinge
CPRCP
SEQ ID NO: 248 Human IgG4 HC (K01316) core hinge
CPSCP
SEQ ID NO: 249 Human IgG1 HC (J00228) and IgG3 HC (M12958) lower hinge
residues
APELLGG
SEQ ID NO: 250 Human IgG2 HC (J00230) lower hinge residues
APPVAG
SEQ ID NO: 251 Human IgG4 HC (K01316) lower hinge residues
APEFLGG
SEQ ID NO: 252 Residues for substitution in the lower hinge
EFEG
SEQ ID NO: 253 Human IgG4 lower hinge residues
EFLG
SEQ ID NO: 254 Human IgG1 lower hinge residues
ELLG
SEQ ID NO: 255 Human IgG2 lower hinge residues
PVAG
SEQ ID NO: 256 Human IgG4 lower hinge residues
EFLGG
SEQ ID NO: 257 Human IgG1 lower hinge residues
ELLGG
SEQ ID NO: 258 Human IgG LALA mutation
EAAGG
SEQ ID NO: 259 Human IgG4 lower hinge residues
FLGG
SEQ ID NO: 260 Human IgG1 lower hinge residues
LLGG

1. An immunotherapeutic protein comprising one or more immunoglobulin heavy chain polypeptide comprising one or more of: a constant heavy chain domain 1 (CH1), a constant heavy chain domain 2 (CH2), a constant heavy chain domain 3 (CH3) and a hinge region, wherein the one or more polypeptide comprises an amino acid substitution at a position corresponding to H429 of the amino acid sequence of human IgG1, IgG2, IgG3 or IgG4 heavy chain (Eu numbering), and wherein the one or more polypeptide comprises at least one C1q binding modification.

2. The immunotherapeutic protein of claim 1, wherein the amino acid substitution at the position corresponding to H429 of the amino acid sequence of a human IgG1, IgG2, IgG3 or IgG4 heavy chain polypeptide is a H to an aromatic amino acid or cyclic amino acid (Eu numbering).

3. The immunotherapeutic protein of claim 1 or claim 2, wherein the substitution at H429 is selected from: H429 substituted with F, H429 substituted with Q, H429 substituted with E, H429 substituted with S, H429 substituted with A, H429 substituted with Y, H429 substituted with L, H429 substituted with V, H429 substituted with G, H429 substituted with H429W, H429 substituted with R and H429 substituted with P (Eu numbering).

4. The immunotherapeutic protein of any one of claims 1 to 3, wherein the substitution at H429 is H429 substituted with F (Eu numbering).

5. The immunotherapeutic protein of any one of claims 1 to 3, wherein the substitution at H429 is H429 substituted with Q (Eu numbering).

6. The immunotherapeutic protein of any one of claims 1 to 3, wherein the substitution at H429 is H429 substituted with E (Eu numbering).

7. The immunotherapeutic protein of any one of claims 1 to 3, wherein the substitution at H429 is H429 substituted with S (Eu numbering).

8. The immunotherapeutic protein of any one of claims 1 to 3, wherein the substitution at H429 is H429 substituted with A (Eu numbering).

9. The immunotherapeutic protein of any one of claims 1 to 3, wherein the substitution at H429 is H429 substituted with Y (Eu numbering).

10. The immunotherapeutic protein of any one of claims 1 to 3, wherein the substitution at H429 is H429 substituted with V (Eu numbering).

11. The immunotherapeutic protein of any one of claims 1 to 10, wherein the immunotherapeutic protein is an antibody that forms oligomers through self-association in solution at physiological pH.

12. The immunotherapeutic protein of claim 11, wherein the immunotherapeutic protein is a hexamer.

13. The immunotherapeutic protein of any one of claims 1 to 10, wherein the immunotherapeutic protein is an antibody that forms oligomers upon binding to a relevant target through “on target” oligomerisation.

14. The immunotherapeutic protein of claim 13, wherein the immunotherapeutic protein forms a hexamer upon binding to a relevant target through “on target” oligomerisation.

15. The immunotherapeutic protein of any one of claims 1 to 14, wherein the C1q binding modification is selected from a modification in C1q group 1, C1q group 2, C1q group 3, C1q group 4 and C1q group 5.

16. The immunotherapeutic protein of claim 15, wherein the C1q binding modification is selected from a modification in C1q Group 1, C1q Group 2 and C1q Group 3 and wherein the modification increases C1q binding compared to a control IgG2 immunotherapeutic protein and/or a control IgG1 immunotherapeutic protein lacking the C1q binding modification.

17. The immunotherapeutic protein of any one of claims 1 to 16, wherein the C1q binding modification increases complement-based lysis compared to a control immunotherapeutic protein lacking the C1q binding modification.

18. The immunotherapeutic protein of claim 15, wherein the C1q binding modification is selected from a modification in C1q Group 4 and C1q Group 5 and wherein the modification decreases C1q binding compared to a control IgG2 immunotherapeutic protein and/or a control IgG1 immunotherapeutic protein lacking the C1q binding modification.

19. The immunotherapeutic protein of any one of claims 1 to 15 and 18, wherein the C1q binding modification decreases complement-based lysis compared to a control immunotherapeutic protein lacking the C1q binding modification.

20. The immunotherapeutic protein of any one of claims 1 to 19, wherein the polypeptide is selected from an IgG1, IgG2, IgG3 or IgG4.

21. The immunotherapeutic protein of any one of claims 1 to 17 and 20, wherein the polypeptide is an IgG1, the C1q binding modification is in the CH1 domain and wherein the modification is a substitution at a position corresponding to S131 of the amino acid sequence of a human IgG1 heavy chain polypeptide substituted with a C (Eu numbering).

22. The immunotherapeutic protein of any one of claims 1 to 20, wherein the polypeptide is an IgG1, IgG2, IgG3 or IgG4, the modification is in the hinge region and wherein the modification is a substitution at a position corresponding to 216-225 or 217-225 of a human IgG1, IgG2, IgG3 or IgG4 heavy chain polypeptide (Eu numbering).

23. The immunotherapeutic protein of claim 22, wherein the substitution in the hinge region is selected from: RKCCVE217-225 substituted with PKSCDKTHT, EPKSCDKTHT216-225 substituted with ERKCCVE, and EPKSCDKTHT216-225 substituted with ESKYGPP.

24. The immunotherapeutic protein of any one of claims 1 to 20, wherein the polypeptide is an IgG2, the modification is in the CH2 domain and wherein the modification is a substitution at a position corresponding to PVA233-236, L328 or S267 of a human IgG1, IgG2, IgG3 or IgG4 heavy chain polypeptide (Eu numbering).

25. The immunotherapeutic protein of claim 24, wherein the substitution in the CH2 domain is selected from: PVA233-236 substituted with EFLG, PVA233-236 substituted with ELLG, and PVA233-236 substituted with EFEG.

26. The immunotherapeutic protein of any one of claims 1 to 15 or 18 to 20, wherein the polypeptide is an IgG1, IgG3 and IgG4, the modification is in the CH2 domain and wherein the modification is a substitution at a position corresponding to L235 of a human IgG1, IgG3 or IgG4 heavy chain polypeptide substituted with an E (Eu numbering).

27. The immunotherapeutic protein of any one of claims 1 to 20, wherein the polypeptide is an IgG1, IgG2, IgG3 or IgG3, the modification is in the CH2 domain and wherein the modification is a substitution at a position corresponding to QYN295-297, YNS296-298, ED269-270, L328 or S267 of a human IgG1, IgG2, IgG3 or IgG4 heavy chain polypeptide (Eu numbering).

28. The immunotherapeutic protein of claim 27, wherein the substitution in the CH2 domain is selected from: QYN295-297 substituted with NST, YNS296-298 substituted with NSS, ED269-270 substituted with AA, L328 substituted with F and S267 substituted with E (Eu numbering).

29. The immunotherapeutic protein of claim 24 or claim 25, wherein the substitution in the CH2 domain is PVA233-236 substituted with EFLG (Eu numbering).

30. The immunotherapeutic protein of claim 24 or claim 25, wherein the substitution in the CH2 domain is PVA233-236 substituted with ELLG (Eu numbering).

31. The immunotherapeutic protein of claim 27 or claim 28, wherein the substitution in the CH2 domain is selected from:
i) 5267 substituted with E and L328 substituted with F (Eu numbering);
ii) S267 substituted with E (Eu numbering); and
iii) L328 substituted with L328 F (Eu numbering).

32. The immunotherapeutic protein of any one of claims 1 to 17 or 20, wherein the polypeptide is an IgG1, IgG2, IgG3 or IgG4 and wherein the modification is in the CH3 domain and wherein the modification is a substitution at a position corresponding to E430 or H435 of a human IgG1, IgG2, IgG3 or IgG4 heavy chain polypeptide (Eu numbering).

33. The immunotherapeutic protein of claim 32, wherein the substitution in the CH3 domain is selected from: E430 substituted with G, and H435 substituted with A.

34. The immunotherapeutic protein of claim 1 to 20, wherein the polypeptide is an IgG3, the modification is in the CH3 domain and wherein the modification is a substitution at a position corresponding to N392, M397 and R435 of the IgG3 heavy chain polypeptide (Eu numbering).

35. The immunotherapeutic protein of claim 34, wherein the substitution in the CH3 domain comprises N392 substituted with K, M397 substituted with V and R435 substituted with H (Eu numbering).

36. The immunotherapeutic protein of any one of claims 1 to 17 and 20, wherein the polypeptide is an IgG2, the C1q binding modification is in the CH1 domain and wherein the modification is a substitution at a position corresponding to C131 of the amino acid sequence of a human IgG1 heavy chain polypeptide substituted with a S (Eu numbering).

37. The immunotherapeutic protein of any one of claims 1 to 17 and 20, wherein the polypeptide is an IgG2 and the C1q binding modification is PVA233-236 substituted with EFLG, C131 substituted with S, and C220 substituted with S.

38. The immunotherapeutic protein of any one of claims 1 to 17 and 20, wherein the polypeptide is an IgG2 and the C1q binding modification is PVA233-236 substituted with EFLG, C131 substituted with S, and C219 substituted with S.

39. The immunotherapeutic protein of any one of claims 1 to 17 and 20, wherein the polypeptide is an IgG2 and the C1q binding modification is PVA233-236 substituted with EFLG, C131 substituted with S, and ERKCCVE substituted with IgG1 216-225EPKSCDKTHT.

40. The immunotherapeutic protein of any one of claims 1 to 15, 19 and 20, wherein the polypeptide is an IgG1 and the C1q binding modification is PVA233-236 substituted with EAAGG.

41. The immunotherapeutic protein of any one of claims 1 to 15, 19 and 20, wherein the polypeptide is an IgG1 and the C1q binding modification is K274 substituted with a Q.

42. The immunotherapeutic protein of any one of claims 1 to 15, 19 and 20, wherein the polypeptide is an IgG2, IgG3 or IgG4 and the C1q binding modification is Q274 substituted with K.

43. The immunotherapeutic protein of any one of claims 1 to 42, wherein the immunotherapeutic protein is an anti-CD20 antibody.

44. The immunotherapeutic protein of any one of claims 1 to 43, wherein the immunotherapeutic protein is selected from: rituximab, ofatumumab, obinutuzumab, isatuximab, trastuzumab and pertuzumab.

45. The immunotherapeutic protein of any one of claims 1 to 44, wherein the C1q binding modification is in the CH1 domain, hinge region, CH2 domain or CH3 domain or a combination thereof.

46. The immunotherapeutic protein of any one of claims 1 to 45, wherein the immunotherapeutic protein is selected from: rituximab (Rit)-IgG2 FLGG HF, Rit-IgG2 FLGG EG, Rit-IgG2 LLGG HF, Isa-IgG2-M1-H429F, Isa-IgG2-M2-H429F, Isa-IgG2-M3-H429F, S2P6-IgG2-M1-H429F, S2P6-IgG2-M2-H429F and S2P6-IgG2-M3-H429F.

47. The immunotherapeutic protein of any one of claims 1 to 46, wherein the immunotherapeutic protein comprises a constant heavy domain 3 (CH3) and a constant heavy domain 2 (CH2) of an immunoglobulin heavy chain, optionally in combination with a lower, core and/or upper hinge sequence.

48. The immunotherapeutic protein of any one of claims 1 to 47, wherein the immunotherapeutic protein is a bispecific immunotherapeutic protein.

49. The immunotherapeutic protein of any one of claims 1 to 48, wherein the immunotherapeutic protein comprises at least two C1q binding modifications.

50. The immunotherapeutic protein of any one of claims 1 to 49, wherein the immunotherapeutic protein comprises at least three C1q binding modifications.

51. The immunotherapeutic protein of any one of claims 1 to 50, wherein the polypeptide comprises a further mutation selected from one or more of:
i) an amino acid substitution at a position corresponding to 274, of the amino acid sequence of human IgG1, IgG2, IgG3 or IgG4 heavy chain (Eu numbering);
ii) an amino acid substitution at a position corresponding to 219 of the amino acid sequence of human IgG1, IgG2, IgG3 or IgG4 heavy chain (Eu numbering);
iii) an amino acid substitution at a position corresponding to 220 of the amino acid sequence of human IgG1, IgG2, IgG3 or IgG4 heavy chain (Eu numbering); and
iv) an amino acid substitution at a position corresponding to 219 and 220 of the amino acid sequence of human IgG1, IgG2, IgG3 or IgG4 heavy chain (Eu numbering).

52. The immunotherapeutic protein of claim 51, wherein the further mutation is a substitution at position corresponding to C219 or a substitution at position corresponding to C220 of an amino acid sequence of human IgG1, IgG2, IgG3 or IgG4 heavy chain (Eu numbering).

53. The immunotherapeutic protein of claim 51, wherein the further mutation is a substitution selected from K274 with Q, Q274 with K, C219 with S, 5219 with C, and C220 with S.

54. The immunotherapeutic protein of any one of claims 1 to 20, 22 to 25, 27 to 32, 36 to 39 and 43 to 53, wherein the polypeptide is an IgG2 and wherein the polypeptide comprises a further mutation that prevents formation of an intra-heavy chain disulfide bond in the upper hinge region of the polypeptide.

55. The immunotherapeutic of claim 54, wherein the intra-heavy chain disulfide bond is between C131 and C219, or between C131 and C220.

56. The immunotherapeutic protein of any one of claims 1 to 20 22 to 25, 27 to 32, 36 to 39 and 43 and 53, wherein the polypeptide is an IgG2 and wherein the polypeptide comprises a further mutation that forms a disulfide bond between the light chain and upper hinge region of the polypeptide.

57. The method of claim 56, wherein the disulfide bond is formed between C214 of the light chain and C219 of the heavy chain or between C214 of the light chain and C220 of the heavy chain.

58. The immunotherapeutic protein of any one of claims 54 to 57, wherein the further mutation is M1, substitution of C131 with S and a substitution of C220 with S.

59. The immunotherapeutic protein of any one of claims 54 to 57, wherein the further mutation is M2, substitution of C131 with S and substitution of C219 with S.

60. The immunotherapeutic protein of claim 54 or 57, wherein the further mutation is M3, a substation of C131 with S and substitution of ERKCCVE in the IgG2 upper hinge with EPKSCDKTHT.

61. The immunotherapeutic protein of any one of claims 1 to 60, wherein the immunotherapeutic protein comprises:
a) H429 substituted with F and wherein the C1q modification is PVA233-236 substituted with EFLG;
b) H429 substituted with F and wherein the C1q modification is PVA233-236 substituted with ELLG;
c) H429 substituted with Q and wherein the C1q modification is PVA233-236 substituted with EFLG;
d) H429 substituted with Q and wherein the C1q modification is PVA233-236 substituted with ELLG;
e) H429 substituted with E and wherein the C1q modification is PVA233-236 substituted with EFLG;
f) H429 substituted with E and wherein the C1q modification is PVA233-236 substituted with ELLG;
g) H429 substituted with S and wherein the C1q modification is PVA233-236 substituted with EFLG;
h) H429 substituted with S and wherein the C1q modification is PVA233-236 substituted with ELLG.
i) H429 substituted with A and wherein the C1q modification is PVA233-236 substituted with EFLG;
j) H429 substituted with A and wherein the C1q modification is PVA233-236 substituted with ELLG.
k) H429 substituted with Y and wherein the C1q modification is PVA233-236 substituted with EFLG;
l) H429 substituted with Y and wherein the C1q modification is PVA233-236 substituted with ELLG;
m) H429 substituted with F, wherein the C1q modification is PVA233-236 substituted with EFLG, C131 is substituted with S, and C220 is substitute with S;
n) H429 substituted with F, wherein the C1q modification is PVA233-236 substituted with EFLG, C131 is substituted with S, and C219 is substitute with S;
o) H429 substituted with F, wherein the C1q modification is PVA233-236 substituted with EFLG, C131 substituted with S, and ERKCCVE in the IgG2 upper hinge substitute with EPKSCDKTHT;
p) H429 substituted with F, wherein the C1q modification is PVA233-236 substituted with ELLG, C131 substituted with S, and C220 substituted with S;
q) H429 substituted with F, wherein the C1q modification is PVA233-236 substituted with ELLG, C131 substituted with S, and C219 substituted with S; and
r) H429 substituted with F, wherein the C1q modification is PVA233-236 substituted with ELLG, C131 substituted with S, and ERKCCVE in the IgG2 upper hinge substituted with IgG1 EPKSCDKTHT.

62. The immunotherapeutic protein of claim 17 or claim 19, wherein the control immunotherapeutic protein is IgG1.

63. The immunotherapeutic protein of claim 17 or claim 19, wherein the control immunotherapeutic protein is human IgG2.

64. An oligomer comprising the immunotherapeutic protein of any one of claims 1 to 63.

65. A nucleic acid encoding the immunotherapeutic protein of any one of claims 1 to 63.

66. The nucleic acid of claim 65, wherein the nucleic acid is selected from an RNA, DNA or combination thereof.

67. The use of the immunotherapeutic protein, oligomer, or nucleic acid of any one of claims 1 to 66 for treating or preventing a disease or condition in a subject, wherein the disease or condition is selected from autoimmune diseases and conditions, cardiovascular disease, neurodegenerative disease, other inflammatory diseases, transplantation condition or rejection, infectious diseases and proliferative diseases.

68. The use of the immunotherapeutic protein, oligomer, or nucleic acid of any one of claims 1 to 66 in the manufacture of a medicament for treating or preventing a disease or condition, wherein the disease or condition is selected from autoimmune diseases and conditions, cardiovascular disease, neurodegenerative disease, other inflammatory diseases, transplantation condition or rejection, infectious diseases and proliferative diseases.

69. A method for treating or preventing a disease or condition, comprising administering to the subject an effective amount of the immunotherapeutic protein, oligomer, or nucleic acid of any one of claims 1 to 66, wherein the disease or condition is selected from autoimmune diseases and conditions, cardiovascular disease, neurodegenerative disease, other inflammatory diseases, transplantation condition or rejection, infectious diseases and proliferative diseases.

70. The use of claim 67 or 68 or method of claim 69, wherein the immunotherapeutic protein or oligomer removes soluble molecules and/or immune complexes from circulation.

71. The use of claim 67 or 68 or method of claim 69, wherein the immunotherapeutic protein enhances death receptor signalling responses.

72. The use of claim 67 or 68 or method of claim 69, wherein the immunotherapeutic protein enhances complement-based lysis when administered to a subject.

73. A pharmaceutical composition or medicament comprising the immunotherapeutic protein, oligomer, or nucleic acid of any one of claims 1 to 66, and a pharmaceutically acceptable carrier, diluent and/or excipient.

74. A kit comprising at least one immunotherapeutic protein, oligomer, or nucleic acid of any one of claims 1 to 66.

75. A method of producing an immunotherapeutic protein of claim 1 to 66, comprising culturing a host cell comprising a construct encoding said protein under conditions suitable for the expression of said protein, and recovering the protein from culture supernatant under conditions of:
(i) mildly acidic pH to recover immunotherapeutic protein in a monomeric form; or
(ii) substantially neutral pH to recover immunotherapeutic protein in an oligomeric form.

76. A method of producing an immunotherapeutic protein of any one of claims 1 to 66, comprising culturing a host cell comprising a construct encoding said protein under conditions suitable for the expression of said protein, and recovering the protein from culture supernatant using a method comprising affinity chromatography using an elution buffer comprising a concentration of arginine of less than 130 mM and at less than or equal to pH 5.0.

77. A method of producing/modifying a immunotherapeutic protein comprising one or more immunoglobulin heavy chain polypeptide comprising: i) substituting the amino acid at a position corresponding to H429 of the amino acid sequence of human IgG1, IgG2, IgG3 or IgG4 heavy chain (Eu numbering) with an aromatic, cyclic or hydrophobic amino acid, and ii) introducing a C1q modification as described in table 5 into the immunotherapeutic protein, wherein the one or more polypeptide comprises one or more of: a constant heavy chain domain 1 (CH1), a constant heavy chain domain 2 (CH2), a constant heavy chain domain 3 (CH3) domain and a hinge region.

78. A method of producing/modifying a immunotherapeutic protein comprising one or more immunoglobulin heavy chain polypeptide comprising: introducing a C1q modification as described in table 5 into the immunotherapeutic protein, wherein the one or more polypeptide comprises one or more of: a constant heavy chain domain 1 (CH1), a constant heavy chain domain 2 (CH2), a constant heavy chain domain 3 (CH3) domain and a hinge region, and wherein the one or more polypeptide comprises an amino acid substitution at a position corresponding to H429 of the amino acid sequence of human IgG1, IgG2, IgG3 or IgG4 heavy chain (Eu numbering) with an aromatic, cyclic or hydrophobic amino acid.

79. A method of producing/modifying a immunotherapeutic protein comprising one or more immunoglobulin heavy chain polypeptide comprising: substituting the amino acid at a position corresponding to H429 of the amino acid sequence of human IgG1, IgG2, IgG3 or IgG4 heavy chain (Eu numbering) with an aromatic, cyclic or hydrophobic amino acid, wherein the one or more polypeptide comprises one or more of: a constant heavy chain domain 1 (CH1), a constant heavy chain domain 2 (CH2), a constant heavy chain domain 3 (CH3) domain and a hinge region, and wherein the one or more polypeptide comprises at least one C1q binding modification.

80. A method of enhancing complement-based lysis mediated by an immunotherapeutic protein comprising one or more immunoglobulin heavy chain polypeptide, the method comprising: i) substituting the amino acid at a position corresponding to H429 of the amino acid sequence of human IgG1, IgG2, IgG3 or IgG4 heavy chain (Eu numbering) with an aromatic, cyclic or hydrophobic amino acid, and ii) introducing a C1q modification as described in table 5 into the immunotherapeutic protein, wherein the polypeptide comprises one or more of: a constant heavy chain domain 5 (CH1), a constant heavy chain domain 2 (CH2), a constant heavy chain domain 3 (CH3) domain and a hinge region.

81. A method of producing/modifying a immunotherapeutic protein comprising one or more immunoglobulin heavy chain polypeptide comprising: i) substituting an amino acid at a position corresponding to H429 of the amino acid sequence of human IgG1, IgG2, IgG3 or IgG4 heavy chain (Eu numbering) with F, Q, E, S, A, Y, L, V, G, W, R or P, and ii) introducing a C1q modification as described in table 5 into the immunotherapeutic protein, wherein the one or more polypeptide comprises one or more of: a constant heavy chain domain 1 (CH1), a constant heavy chain domain 2 (CH2), a constant heavy chain domain 3 (CH3) domain and a hinge region.

82. A method of producing/modifying a immunotherapeutic protein comprising one or more immunoglobulin heavy chain polypeptide comprising: introducing a C1q modification as described in table 5 into the immunotherapeutic protein, wherein the one or more polypeptide comprises one or more of: a constant heavy chain domain 1 (CH1), a constant heavy chain domain 2 (CH2), a constant heavy chain domain 3 (CH3) domain and a hinge region, and wherein the one or more polypeptide comprises an amino acid substitution at a position corresponding to H429 of the amino acid sequence of human IgG1, IgG2, IgG3 or IgG4 heavy chain (Eu numbering) with F, Q, E, S, A, Y, L, V, G, W, R or P.

83. A method of producing/modifying a immunotherapeutic protein comprising one or more immunoglobulin heavy chain polypeptide comprising: substituting an amino acid at a position corresponding to H429 of the amino acid sequence of human IgG1, IgG2, IgG3 or IgG4 heavy chain (Eu numbering) with F, Q, E, S, A, Y, L, V, G, W, R or P, wherein the one or more polypeptide comprises one or more of: a constant heavy chain domain 1 (CH1), a constant heavy chain domain 2 (CH2), a constant heavy chain domain 3 (CH3) domain and a hinge region, and wherein the one or more polypeptide comprises at least one C1q binding modification.

84. A method of enhancing complement-based lysis mediated by an immunotherapeutic protein comprising one or more immunoglobulin heavy chain polypeptide, the method comprising: i) substituting an amino acid at a position corresponding to H429 of the amino acid sequence of human IgG1, IgG2, IgG3 or IgG4 heavy chain (Eu numbering) with F, Q, E, S, A, Y, L, V, G, W, R or P, and ii) introducing a C1q modification as described in table 5 into the immunotherapeutic protein, wherein the polypeptide comprises one or more of: a constant heavy chain domain 5 (CH1), a constant heavy chain domain 2 (CH2), a constant heavy chain domain 3 (CH3) domain and a hinge region.

85. The method of any one of claims 77 to 84, wherein the C1q binding modification is selected from a modification in C1q group 1, C1q group 2, C1q group 3, C1q group 4 and C1q group 5.

86. An immunotherapeutic protein comprising modified C1q binding wherein the immunotherapeutic protein comprises one or more immunoglobulin heavy chain polypeptide comprising one or more of: a constant heavy chain domain 1 (CH1), a constant heavy chain domain 2 (CH2), a constant heavy chain domain 3 (CH3) domain and a hinge region, wherein the one or more polypeptide comprises substitution of an amino acid at a position corresponding to H429 of the amino acid sequence of human IgG1, IgG2, IgG3 or IgG4 heavy chain (Eu numbering) with F, Q, E, S, A, Y, L, V, G, W, R or P.

87. A method of producing/modifying C1q binding of an immunotherapeutic protein comprising one or more immunoglobulin heavy chain polypeptide, the method comprising substituting an amino acid at a position corresponding to H429 of the amino acid sequence of human IgG1, IgG2, IgG3 or IgG4 heavy chain (Eu numbering) with F, Q, E, S, A, Y, L, V, G, W, R or P.

88. A method of enhancing complement-based lysis mediated by an immunotherapeutic protein comprising one or more immunoglobulin heavy chain polypeptide, the method comprising: substituting an amino acid at a position corresponding to H429 of the amino acid sequence of human IgG1, IgG2, IgG3 or IgG4 heavy chain (Eu numbering) with F, Q, E, S, A, Y, L, V, G, W, R or P.

89. The method of any one of claims 77, 79, 80, 81, 83, 84, 85, 87 and 88, the substitution at H429 is selected from: H429 substituted with F, H429 substituted with Q, H429 substituted with E, H429 substituted with S, H429 substituted with A, H429 substituted with Y, H429 substituted with L, H429 substituted with V, H429 substituted with G, H429 substituted with H429W, H429 substituted with R and H429 substituted with P (Eu numbering).

90. The method of claim 89, wherein the substitution at H429 is H429 substituted with F (Eu numbering).


SEQUENCE LISTING (260 sequences)

SEQ ID NO: 1 - protein, 968 aa, synthetic construct
ETGSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDRAYEWNDNEMYLFRSSVAYAMRQYFLKVKNQMILFGEEDVRVANLKPRISFNFFVTAPKNVSDIIPRTEVEKAIRMSRSRINDAFRLNDNFCEUNUMBERINGSLEFLGIQPTLGPPNQPPVSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 832 aa, synthetic construct
ETGSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSGSGSGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 330 aa, synthetic construct [sequence omitted]

SEQ ID NO: 4 - protein, 377 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 5 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 7 - protein, 353 aa, synthetic construct
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 8 - protein, 340 aa, synthetic construct
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 9 - protein, 110 aa, synthetic construct
GPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 10 - protein, 131 aa, synthetic construct
GVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 11 - protein, 117 aa, synthetic construct
AAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 12 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1353 bp, synthetic construct
  source 1..1353: mol_type = other DNA
gaagtgcagctggttgaatctggcggcggactggttcaaccaggcggatctctgagactgagctgtgccgccagcggcttcaacatcaaggacacctacatccactgggtccgacaggcccctggcaaaggacttgaatgggtcgccagaatctaccccaccaacggctacaccagatacgccgactctgtgaagggcagattcaccatcagcgccgacaccagcaagaacaccgcctacctgcagatgaacagcctgagagccgaggacaccgccgtgtactactgtagtagatggggaggcgacggcttctacgctatggattattggggccagggcaccctggtcacagttagctctgcgtcgacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaatga

SEQ ID NO: 14 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - DNA, 645 bp, synthetic construct
  source 1..645: mol_type = other DNA
gatatccagatgacacagagccccagcagcctgtctgcctctgtgggagacagagtgaccatcacctgtagagccagccaggacgtgaacacagccgtggcttggtatcagcagaagcctggcaaggcccctaagctgctgatctacagcgccagctttctgtacagcggcgtgcccagcagattcagcggaagcagaagcggcaccgacttcaccctgaccataagcagtctgcagcccgaggacttcgccacctactactgtcagcagcactacaccacacctccaaccttcggccagggtaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgctaa

SEQ ID NO: 16 - protein, 452 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 1359 bp, synthetic construct
  source 1..1359: mol_type = other DNA
gaagtgcagctggttgaatctggcggcggactggttcagcctggcagatctctgagactgagctgtgccgccagcggcttcaccttcaacgattacgccatgcactgggtccgacaggcccctggaaaaggccttgaatgggtgtccaccatcagctggaacagcggctctatcggctacgccgattccgtgaagggcagattcaccatctccagagacaacgccaagaagtccctgtacctgcagatgaacagcctgagagccgaggacacagccctgtactactgcgccaaggacatccagtacggcaactactactacggcatggacgtgtggggccagggcacactggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaatga

SEQ ID NO: 18 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 645 bp, synthetic construct
  source 1..645: mol_type = other DNA
gaaattgtgctgacacagagccccgccacactgtcactttctccaggcgaaagagccacactgagctgcagagccagccagagcgtgtcctcttacctggcctggtatcagcagaagcctggacaggctccccggctgctgatctacgatgccagcaatagagccaccggcattcccgccagattttctggctctggaagcggcaccgacttcaccctgaccataagcagcctggaacctgaggacttcgccgtgtactactgccagcagagaagcaactggcccatcacctttggccagggtaccagactggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgctaa

SEQ ID NO: 20 - protein, 452 aa, synthetic construct
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 1359 bp, synthetic construct
  source 1..1359: mol_type = other DNA
gaagttcagctgctggaatctggcggcggactggttcaaccaggcggatctctgagactgagctgtgccgtgtccggcttcaccttcaacagcttcgccatgagctgggtccgacaggcccctggaaaaggccttgaatgggtgtccgccatctctggctctggcggaggcacatattacgccgactctgtgaagggcagattcaccatcagccgggacaacagcaagaacaccctgtacctgcagatgaacagcctgagagccgaggataccgccgtgtacttctgcgccaaggacaagatcctttggttcggcgagcccgtgttcgattattggggccagggcaccctggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagagagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaatga

SEQ ID NO: 22 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 645 bp, synthetic construct
  source 1..645: mol_type = other DNA
gaaattgtgctgacacagagccccgccacactgtcactttctccaggcgaaagagccacactgagctgcagagccagccagagcgtgtcctcttacctggcctggtatcagcagaagcctggacaggctccccggctgctgatctacgatgccagcaatagagccaccggcattcccgccagattttctggctctggaagcggcaccgacttcaccctgaccataagcagcctggaacctgaggacttcgccgtgtactactgccagcagagaagcaactggcctcctacctttggccagggtaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgctaa

SEQ ID NO: 24 - protein, 449 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 1350 bp, synthetic construct
  source 1..1350: mol_type = other DNA
gaagtgcagctggttgaatctggcggcggactggttcaaccaggcggatctctgagactgagctgtgccgccagcggcttcaccttcaccgactacacaatggactgggtccgacaggcccctggcaaaggacttgaatgggtcgccgacgtgaaccccaatagcggcggcagcatctacaaccagcggttcaagggcagattcaccctgagcgtggacagaagcaagaacaccctgtacctgcagatgaacagcctgagagccgaggacaccgccgtgtactactgcgccagaaatctgggccccagcttctacttcgattattggggccagggcaccctggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaatga

SEQ ID NO: 26 - protein, 214 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - DNA, 645 bp, synthetic construct
  source 1..645: mol_type = other DNA
gatatccagatgacacagagccccagcagcctgtctgcctctgtgggagacagagtgaccatcacctgtaaagccagccaggacgtgtccatcggcgtggcatggtatcagcagaagcctggcaaggcccctaagctgctgatctacagcgccagctacagatacaccggcgtgcccagcagattttctggcagcggctctggcaccgacttcaccctgaccatatctagcctgcagcctgaggacttcgccacctactactgccagcagtactacatctacccctacaccttcggccagggtaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgctaa

SEQ ID NO: 28 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 1356 bp, synthetic construct
  source 1..1356: mol_type = other DNA
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggcaccaccgtgacagtttctgccgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggatgaactgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaatga

SEQ ID NO: 30 - protein, 213 aa, synthetic construct
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 642 bp, synthetic construct
  source 1..642: mol_type = other DNA
caaattgtgctgtctcagagccccgccatcctgagtgcttctccaggcgagaaagtgaccatgacctgcagagccagcagcagcgtgtcctacatccactggttccagcagaagcctggcagcagccctaagccttggatctacgccacaagcaatctggccagcggcgtgccagtcagattttctggctctggcagcggcaccagctacagcctgacaatcagcagagtggaagccgaggatgccgccacctactactgtcagcagtggaccagcaatcctcctacctttggcggcggtaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgctaa

SEQ ID NO: 32 - protein, 446 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 1341 bp, synthetic construct
  source 1..1341: mol_type = other DNA
gaggtgcagcttcaggagtcaggacctagcctcgtgaaaccttctcagactctgtccctcacatgttctgtcactggcgactccatcaccagtggttactggaactggatccggcaattcccagggaataaacttgagtacatgggtttcataaattacagtggtaacacttactacaatccatctctcagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 34 - protein, 442 aa, synthetic construct
EVQLQESGPSLVKPSQTLSLTCSVTGDSITSGYWNWIRQVPGNKLEYMGFINYSGNTYYNPSLRSRISITRDTSKNQYFLHLNSVTTEDTATYYCARANWDVFAYWGQGTLVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSLGK

SEQ ID NO: 35 - DNA, 1329 bp, synthetic construct
  source 1..1329: mol_type = other DNA
gaggtgcagttgcaggaatctggtcccagtctagttaagcccagccagacactgagcctgacatgtagtgtgaccggtgacagcattacaagcggctactggaactggatcagacaggtgccaggaaacaaactcgaatacatggggttcatcaattacagtggcaatacttactataatcccagcctgagaagcagaatttctataaccagagacaccagcaaaaaccagtactttctgcacctgaacagcgtaacaacggaagacaccgccacctattactgcgccagggccaactgggatgtgttcgcatactggggccagggcactctggtgacggtgagcgccgcgtcgacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggtacccagacctatacatgcaatgtcgatcataaacccagtaacacaaaggtagacaagacagtcgaaagaaaatgctgtgtggagtgccctccttgccccgcccccccagtggccgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcttaggaaaatga

SEQ ID NO: 36 - protein, 220 aa, synthetic construct
DIVMSQSPSSLAVSVGEKVTMSCKSSQSLLYSSNQKNYLAWYQRKPGQSPKLLIYWASTRESGVPDRFTGSGSGTDFTLTISSVKAEDLAVYYCQHYYSSPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - DNA, 660 bp, synthetic construct
  source 1..660: mol_type = other DNA
gacatagtgatgtcacagagcccaagcagccttgcagtgtctgttggtgagaaggtgaccatgagctgcaagtcaagccagagcctgctctatagcagcaatcagaagaactatttggcctggtatcagagaaaacccggccagagccctaagctccttatttattgggctagcacacgcgagtccggcgtgccggacagatttaccggcagcggtagcggcaccgattttactttgaccatctcgtctgtaaaggccgaagacctggcagtgtattactgtcaacattattactcatctccctacactttcggaggggggaccaaactggaaattaagcgtacggttgctgccccttctgtcttcatcttccctcccagcgatgaacagctgaaaagtgggacagcgagtgtagtgtgcctgctaaacaatttttacccacgggaggccaaagtgcagtggaaagtggacaacgctctgcaaagtggaaattctcaggagtccgtcacagagcaggactcgaaggacagcacttacagcttgtcaagcaccctgacgctgagcaaagccgattacgagaagcacaaggtgtacgcatgcgaagttacgcaccaaggactgagtagcccagtcacaaagagcttcaacaggggggaatgt

SEQ ID NO: 38 - protein, 498 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKLREEQYNSTFRVVSVLTVLHQDWLNGKEYKCKVSNKALPAPIEKTISKTKGQPREPQVYTLPPSREEMTKNQVSLTCLVKGFYPSDIAVEWESNGQPENNYKTTPPVLDSDGSFFLYSKLTVDKSRWQQGNIFSCSVMHEALHNHYTQKSLSLSPGK

SEQ ID NO: 39 - DNA, 1494 bp, synthetic construct
  source 1..1494: mol_type = other DNA
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggtaccaccgtgacagtttctgccgctagcacaaaaggccctagcgtctttccactcgccccatgttctagaagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggcacccagacctatacctgcaatgtcaatcataaacccagtaacacaaaggtagacaagcgggtcgaactgaaaacccccctgggcgacaccacccacacctgtcctagatgccccgagcccaagtcctgcgatacccctccaccttgccccaggtgccctgagcctaagagctgtgataccccccctccctgccctcggtgtccagagcctaagtcttgtgacaccccacccccatgtccaaggtgccccgcccccgaactcctggggggaccttcggtttttctttttccacctaaaccgaaagacaccctgatgatcagcaggacaccagaagtgacatgtgtcgtcgtggacgtatcccatgaggacccggaggtgcagttcaagtggtatgttgatggtgtggaagtgcataatgcaaaaactaagctgcgggaggaacagtacaatagcacattcagagtagttagcgttctgactgtcctgcaccaagattggctgaacgggaaggaatacaagtgtaaggtgagcaacaaagccctgcccgctccaattgagaaaacaatttctaagaccaaaggacagccccgggagccacaggtgtatactctgccgcctagcagggaagagatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacatcttcagttgctcagtgatgcacgaagcgctgcacaatcattatactcagaaaagcctgagcttgagcccgggaaaa

SEQ ID NO: 40 - protein, 448 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - DNA, 1344 bp, synthetic construct
  source 1..1344: mol_type = other DNA
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggtaccaccgtgacagtttctgccgctagcacaaaaggccctagcgtctttccactcgccccatgttccagaagcacctctgaaagtactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggcaccaagacctatacatgcaatgtcgaccataaacccagtaacacaaaggtagacaagcgagtcgaaagcaagtacggccccccctgccctagctgccccgcccccgaatttctggggggaccttcggtttttctttttccacctaaaccgaaagacaccctgatgatcagcaggacaccagaagtgacatgtgtcgtcgtggacgtatcccaggaggacccggaggtgcagttcaactggtatgttgatggtgtggaagtgcataatgcaaaaactaagccacgggaggaacagttcaatagcacatacagagtagttagcgttctgactgtcctgcaccaagattggctgaacgggaaggaatacaagtgtaaggtgagcaacaaaggcctgcccagtagcattgagaaaacaatttctaaggccaaaggacagccccgggagccacaggtgtatactctgccgcctagccaagaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcacggctgaccgtggacaaaagcagatggcaggagggaaacgttttcagttgctcagtgatgcacgaagcgctgcacaatcattatactcagaaaagcctgagcttgagcttaggaaaa

SEQ ID NO: 42 - protein, 450 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 1353 bp, synthetic construct
  source 1..1353: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggcacctccgtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaatga

SEQ ID NO: 44 - protein, 214 aa, synthetic construct
DIVMTQSHLSMSTSLGDPVSITCKASQDVSTVVAWYQQKPGQSPRRLIYSASYRYIGVPDRFTGSGAGTDFTFTISSVQAEDLAVYYCQQHYSPPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - DNA, 645 bp, synthetic construct
  source 1..645: mol_type = other DNA
gatatcgtgatgacacagagccacctgagcatgagcaccagcctgggagatcccgtgtccatcacctgtaaagccagccaggatgtgtccaccgtggtggcctggtatcagcagaagcctggccagtctcctcggcggctgatctactctgccagctacagatatatcggcgtgcccgacagattcaccggatctggcgctggcaccgacttcacctttaccatcagctctgtgcaggccgaggacctggccgtgtactattgtcagcagcactacagccctccatacacctttggcggcggtaccaagctggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgctaa

SEQ ID NO: 46 - protein, 447 aa, synthetic construct
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - DNA, 1341 bp, synthetic construct
  source 1..1341: mol_type = other DNA
gaagtgcagctgcaacagtctggcgccgaggttgtgaaacctggcgcctctgtgaagctgagctgtaaagccagcggcttcaacatcaaggacaccttcatccactgggtcaagcaggcccctggacagggactcgaatggatcggcagaatcgaccccgccaacaccaacactaagtacgaccccaagttccagggcaaagccaccatcaccaccgacaccagcagcaacaccgcctacatggaactgagcagcctgagaagcgaggacaccgccgtgtactattgtgtgcggggcctgtacacctactacttcgattattggggccagggtaccctggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggcacccagacctatatctgcaatgtcaatcataaacccagtaacacaaaggtagacaagagagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccggga

SEQ ID NO: 48 - protein, 214 aa, synthetic construct
EIVMTQSPATLSVSPGERATLSCRASQSISNNLHWYQQKPGQAPRLLIKFASQSITGIPARFSGSGSGTEFTLTISSLQSEDFAVYYCQQGNSWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - DNA, 642 bp, synthetic construct
  source 1..642: mol_type = other DNA
gagattgtgatgacacagagccccgccacactgtccgtttctccaggcgaaagagccacactgagctgcagagccagccagagcatcagcaacaacctgcactggtatcagcagaagcccggacaggctcccagactgctgatcaagtttgccagccagtccatcacaggcatccccgccagattttctggcagcggctctggcaccgagttcaccctgacaatcagcagcctgcagagcgaggacttcgccgtgtactattgccagcagggcaacagctggccctacaccttcggccagggtaccaagctggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgc

SEQ ID NO: 50 - protein, 443 aa, synthetic construct
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 51 - DNA, 1329 bp, synthetic construct
  source 1..1329: mol_type = other DNA
gaagtgcagctgcaacagtctggcgccgaggttgtgaaacctggcgcctctgtgaagctgagctgtaaagccagcggcttcaacatcaaggacaccttcatccactgggtcaagcaggcccctggacagggactcgaatggatcggcagaatcgaccccgccaacaccaacactaagtacgaccccaagttccagggcaaagccaccatcaccaccgacaccagcagcaacaccgcctacatggaactgagcagcctgagaagcgaggacaccgccgtgtactattgtgtgcggggcctgtacacctactacttcgactattggggccagggtaccctggtcacagttagctctgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgcccccccagtggccgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccggga

SEQ ID NO: 52 - protein, 447 aa, synthetic construct
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - DNA, 1341 bp, synthetic construct
  source 1..1341: mol_type = other DNA
caggttcagctggttcagtctggcgccgaagtgaagaaacctggcgcctctgtgaaggtgtcctgcaaggccagcggcttcaacatcaaggacacccacatgcactgggtccgacaggctccaggacagagactggaatggatcggcagaatcgaccccgccaacggcaacaccgagtacgaccagaaattccagggcagagtgaccatcaccgtggacacatctgccagcaccgcctacatggaactgagcagcctgagaagcgaggacaccgccgtgtactactgtgccagatggggcaccaacgtgtacttcgcctattggggccagggtaccctggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggcacccagacctatatctgcaatgtcaatcataaacccagtaacacaaaggtagacaagagagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccggga

SEQ ID NO: 54 - protein, 213 aa, synthetic construct
DIQLTQSPSSLSASVGDRVTITCSASSSVSYMYWYQQKPGKAPKPWIYRTSNLASGVPSRFSGSGSGTDFTLTISSLQPEDFATYYCQQYHSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - DNA, 639 bp, synthetic construct
  source 1..639: mol_type = other DNA
gatatccagctgacacagagccctagcagcctgtctgcctctgtgggcgacagagtgaccatcacatgtagcgccagcagcagcgtgtcctacatgtactggtatcagcagaagcccggcaaggcccctaagccttggatctacagaaccagcaatctggccagcggcgtgcccagcagattttctggttctggcagcggcaccgacttcaccctgaccatatctagcctgcagcctgaggacttcgccacctactactgccagcagtaccacagctaccctccaacctttggcggcggtaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgc

SEQ ID NO: 56 - protein, 443 aa, synthetic construct
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 57 - DNA, 1329 bp, synthetic construct
  source 1..1329: mol_type = other DNA
caggttcagctggttcagtctggcgccgaagtgaagaaacctggcgcctctgtgaaggtgtcctgcaaggccagcggcttcaacatcaaggacacccacatgcactgggtccgacaggctccaggacagagactggaatggatcggcagaatcgaccccgccaacggcaacaccgagtacgaccagaaattccagggcagagtgaccatcaccgtggacacatctgccagcaccgcctacatggaactgagcagcctgagaagcgaggacaccgccgtgtactactgtgccagatggggcaccaacgtgtacttcgcctattggggacagggtaccctcgtgaccgttagctctgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgcccccccagtggccgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccggga

SEQ ID NO: 58 - protein, 439 aa, synthetic construct
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAG

SEQ ID NO: 59 - DNA, 1317 bp, synthetic construct
  source 1..1317: mol_type = other DNA
gaagttcagctgcaacagtctggcgccgaggttgtgaaacctggcgcctctgtgaagctgagctgtaaagccagcggcttcaacatcaaggacaccttcatccactgggtcaagcaggcccctggacagggactcgagtggatcggaagaatcgaccccgccaacaccaacactaagtacgaccccaagttccagggcaaagccaccatcaccaccgacaccagcagcaacaccgcctacatggaactgagcagcctgagaagcgaggacaccgccgtgtactattgtgtgcggggcctgtacacctactacttcgactattggggccagggtaccctcgtgaccgttagctctgctagccctacaagccccaaggtgttccctctgagcctggatagcacaccccaggacggaaatgtggtggtggcttgtctggtgcagggattcttcccacaagagcccctgtccgtgacttggagcgagagcggacagaatgtgaccgccagaaacttcccacctagccaggatgccagcggcgatctgtacacaacaagcagccagctgaccctgcctgccacacagtgtcctgatggcaagagcgtgacctgccacgtgaagcactacacaaaccccagccaggacgtgaccgtgccttgtcctgttcctcctccacctccatgctgtcaccctagactgagcctgcacagacccgctctggaagatctgctgctgggctctgaggccaacctgacctgtacactgaccggcctgagagatgcctctggcgccacctttacatggacacctagcagcggaaagtccgccgttcagggacctcctgagagggatctgtgtggctgttacagcgtgtcctctgtgctgcctggatgtgcccagccttggaatcacggcgagacattcacctgtaccgccgctcatcccgagctgaaaacacccctgaccgccaacatcaccaagagcggcaataccttcagacccgaagtgcatctgctgcctccaccatctgaagaactggccctgaacgagctggtgaccctgacatgtctggccaggggcttcagccctaaggatgtgctcgttagatggctgcagggcagccaagagctgcccagagagaagtatctgacctgggccagtcgacaggaaccttctcagggcaccacaacctttgccgtgaccagcattctgagagtggccgccgaggattggaagaagggcgacacctttagctgcatggtcggacacgaagcgctgcccctggctttcacccagaaaaccatcgatagactggccggc

SEQ ID NO: 60 - protein, 439 aa, synthetic construct
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAG

SEQ ID NO: 61 - DNA, 1317 bp, synthetic construct
  source 1..1317: mol_type = other DNA
caggttcagctggttcagtctggcgccgaagtgaagaaacctggcgcctctgtgaaggtgtcctgcaaggccagcggcttcaacatcaaggacacccacatgcactgggtccgacaggctccaggacagagactggaatggatcggcagaatcgaccccgccaacggcaacaccgagtacgaccagaaattccagggcagagtgaccatcaccgtggacacatctgccagcaccgcctacatggaactgagcagcctgagaagcgaggacaccgccgtgtactactgtgccagatggggcaccaacgtgtacttcgcctattggggacagggtaccctcgtgaccgttagctctgctagccctacaagccccaaggtgttccctctgagcctggatagcacaccccaggacggaaatgtggtggtggcttgtctggtgcagggattcttcccacaagagcccctgtccgtgacttggagcgagagcggacagaatgtgaccgccagaaacttcccacctagccaggatgccagcggcgatctgtacacaacaagcagccagctgaccctgcctgccacacagtgtcctgatggcaagagcgtgacctgccacgtgaagcactacacaaaccccagccaggacgtgaccgtgccttgtcctgttcctcctccacctccatgctgtcaccctagactgagcctgcacagacccgctctggaagatctgctgctgggctctgaggccaacctgacctgtacactgaccggcctgagagatgcctctggcgccacctttacatggacacctagcagcggaaagtccgccgttcagggacctcctgagagggatctgtgtggctgttacagcgtgtcctctgtgctgcctggatgtgcccagccttggaatcacggcgagacattcacctgtaccgccgctcatcccgagctgaaaacacccctgaccgccaacatcaccaagagcggcaataccttcagacccgaagtgcatctgctgcctccaccatctgaagaactggccctgaacgagctggtgaccctgacatgtctggccaggggcttcagccctaaggatgtgctcgttagatggctgcagggcagccaagagctgcccagagagaagtatctgacctgggccagtcgacaggaaccttctcagggcaccacaacctttgccgtgaccagcattctgagagtggccgccgaggattggaagaagggcgacacctttagctgcatggtcggacacgaagcgctgcccctggctttcacccagaaaaccatcgatagactggccggc

SEQ ID NO: 62 - protein, 447 aa, synthetic construct
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMFEALHNHYTQKSLSLSPG

SEQ ID NO: 63 - DNA, 1341 bp, synthetic construct
  source 1..1341: mol_type = other DNA
gaagtgcagctgcaacagtctggcgccgaggttgtgaaacctggcgcctctgtgaagctgagctgtaaagccagcggcttcaacatcaaggacaccttcatccactgggtcaagcaggcccctggacagggactcgaatggatcggcagaatcgaccccgccaacaccaacactaagtacgaccccaagttccagggcaaagccaccatcaccaccgacaccagcagcaacaccgcctacatggaactgagcagcctgagaagcgaggacaccgccgtgtactattgtgtgcggggcctgtacacctactacttcgattattggggccagggtaccctggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggcacccagacctatatctgcaatgtcaatcataaacccagtaacacaaaggtagacaagagagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccggga

SEQ ID NO: 64 - protein, 443 aa, synthetic construct
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMFEALHNHYTQKSLSLSPG

SEQ ID NO: 65 - DNA, 1329 bp, synthetic construct
  source 1..1329: mol_type = other DNA
gaagtgcagctgcaacagtctggcgccgaggttgtgaaacctggcgcctctgtgaagctgagctgtaaagccagcggcttcaacatcaaggacaccttcatccactgggtcaagcaggcccctggacagggactcgaatggatcggcagaatcgaccccgccaacaccaacactaagtacgaccccaagttccagggcaaagccaccatcaccaccgacaccagcagcaacaccgcctacatggaactgagcagcctgagaagcgaggacaccgccgtgtactattgtgtgcggggcctgtacacctactacttcgactattggggccagggtaccctggtcacagttagctctgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgcccccccagtggccgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccggga

SEQ ID NO: 66 - protein, 447 aa, synthetic construct
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMFEALHNHYTQKSLSLSPG

SEQ ID NO: 67 - DNA, 1341 bp, synthetic construct
  source 1..1341: mol_type = other DNA
caggttcagctggttcagtctggcgccgaagtgaagaaacctggcgcctctgtgaaggtgtcctgcaaggccagcggcttcaacatcaaggacacccacatgcactgggtccgacaggctccaggacagagactggaatggatcggcagaatcgaccccgccaacggcaacaccgagtacgaccagaaattccagggcagagtgaccatcaccgtggacacatctgccagcaccgcctacatggaactgagcagcctgagaagcgaggacaccgccgtgtactactgtgccagatggggcaccaacgtgtacttcgcctattggggccagggtaccctggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggcacccagacctatatctgcaatgtcaatcataaacccagtaacacaaaggtagacaagagagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccggga

SEQ ID NO: 68 - protein, 443 aa, synthetic construct
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMFEALHNHYTQKSLSLSPG

SEQ ID NO: 69 - DNA, 1329 bp, synthetic construct
  source 1..1329: mol_type = other DNA
caggttcagctggttcagtctggcgccgaagtgaagaaacctggcgcctctgtgaaggtgtcctgcaaggccagcggcttcaacatcaaggacacccacatgcactgggtccgacaggctccaggacagagactggaatggatcggcagaatcgaccccgccaacggcaacaccgagtacgaccagaaattccagggcagagtgaccatcaccgtggacacatctgccagcaccgcctacatggaactgagcagcctgagaagcgaggacaccgccgtgtactactgtgccagatggggcaccaacgtgtacttcgcctattggggacagggtaccctcgtgaccgttagctctgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgcccccccagtggccgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccggga

SEQ ID NO: 70 - protein, 439 aa, synthetic construct
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGFEALPLAFTQKTIDRLAG

SEQ ID NO: 71 - DNA, 1317 bp, synthetic construct
  source 1..1317: mol_type = other DNA
gaagttcagctgcaacagtctggcgccgaggttgtgaaacctggcgcctctgtgaagctgagctgtaaagccagcggcttcaacatcaaggacaccttcatccactgggtcaagcaggcccctggacagggactcgagtggatcggaagaatcgaccccgccaacaccaacactaagtacgaccccaagttccagggcaaagccaccatcaccaccgacaccagcagcaacaccgcctacatggaactgagcagcctgagaagcgaggacaccgccgtgtactattgtgtgcggggcctgtacacctactacttcgactattggggccagggtaccctcgtgaccgttagctctgctagccctacaagccccaaggtgttccctctgagcctggatagcacaccccaggacggaaatgtggtggtggcttgtctggtgcagggattcttcccacaagagcccctgtccgtgacttggagcgagagcggacagaatgtgaccgccagaaacttcccacctagccaggatgccagcggcgatctgtacacaacaagcagccagctgaccctgcctgccacacagtgtcctgatggcaagagcgtgacctgccacgtgaagcactacacaaaccccagccaggacgtgaccgtgccttgtcctgttcctcctccacctccatgctgtcaccctagactgagcctgcacagacccgctctggaagatctgctgctgggctctgaggccaacctgacctgtacactgaccggcctgagagatgcctctggcgccacctttacatggacacctagcagcggaaagtccgccgttcagggacctcctgagagggatctgtgtggctgttacagcgtgtcctctgtgctgcctggatgtgcccagccttggaatcacggcgagacattcacctgtaccgccgctcatcccgagctgaaaacacccctgaccgccaacatcaccaagagcggcaataccttcagacccgaagtgcatctgctgcctccaccatctgaagaactggccctgaacgagctggtgaccctgacatgtctggccaggggcttcagccctaaggatgtgctcgttagatggctgcagggcagccaagagctgcccagagagaagtatctgacctgggccagtcgacaggaaccttctcagggcaccacaacctttgccgtgaccagcattctgagagtggccgccgaggattggaagaagggcgacacctttagctgcatggtcggattcgaagcgctgcccctggctttcacccagaaaaccatcgatagactggccggc

SEQ ID NO: 72 - protein, 439 aa, synthetic construct
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGFEALPLAFTQKTIDRLAG

SEQ ID NO: 73 - DNA, 1317 bp, synthetic construct
  source 1..1317: mol_type = other DNA
caggttcagctggttcagtctggcgccgaagtgaagaaacctggcgcctctgtgaaggtgtcctgcaaggccagcggcttcaacatcaaggacacccacatgcactgggtccgacaggctccaggacagagactggaatggatcggcagaatcgaccccgccaacggcaacaccgagtacgaccagaaattccagggcagagtgaccatcaccgtggacacatctgccagcaccgcctacatggaactgagcagcctgagaagcgaggacaccgccgtgtactactgtgccagatggggcaccaacgtgtacttcgcctattggggacagggtaccctcgtgaccgttagctctgctagccctacaagccccaaggtgttccctctgagcctggatagcacaccccaggacggaaatgtggtggtggcttgtctggtgcagggattcttcccacaagagcccctgtccgtgacttggagcgagagcggacagaatgtgaccgccagaaacttcccacctagccaggatgccagcggcgatctgtacacaacaagcagccagctgaccctgcctgccacacagtgtcctgatggcaagagcgtgacctgccacgtgaagcactacacaaaccccagccaggacgtgaccgtgccttgtcctgttcctcctccacctccatgctgtcaccctagactgagcctgcacagacccgctctggaagatctgctgctgggctctgaggccaacctgacctgtacactgaccggcctgagagatgcctctggcgccacctttacatggacacctagcagcggaaagtccgccgttcagggacctcctgagagggatctgtgtggctgttacagcgtgtcctctgtgctgcctggatgtgcccagccttggaatcacggcgagacattcacctgtaccgccgctcatcccgagctgaaaacacccctgaccgccaacatcaccaagagcggcaataccttcagacccgaagtgcatctgctgcctccaccatctgaagaactggccctgaacgagctggtgaccctgacatgtctggccaggggcttcagccctaaggatgtgctcgttagatggctgcagggcagccaagagctgcccagagagaagtatctgacctgggccagtcgacaggaaccttctcagggcaccacaacctttgccgtgaccagcattctgagagtggccgccgaggattggaagaagggcgacacctttagctgcatggtcggattcgaagcgctgcccctggctttcacccagaaaaccatcgatagactggccggc

SEQ ID NO: 74 - protein, 453 aa, synthetic construct
EVQLLESGGGLVQPGGSLRLSCAASGFTFDDYGMSWVRQAPGKGLEWVSDISWNGGKTHYVDSVKGQFTISRDNSKNTLYLQMNSLRAEDTAVYYCARGSLFHDSSGFYFGHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - DNA, 1359 bp, synthetic construct
  source 1..1359: mol_type = other DNA
gaagtgcagctgctggaatctggcggcggacttgttcaaccaggcggctctctgagactgagctgtgccgcttccggcttcaccttcgacgattacggcatgagctgggtccgacaggcccctggaaaaggccttgaatgggtgtccgacatcagctggaatggcggcaagacccactacgtggactctgtgaagggccagttcaccatcagccgggacaacagcaagaacaccctgtacctgcagatgaacagcctgagagccgaggacaccgccgtgtactattgtgccagaggcagcctgttccacgacagcagcggcttctattttggccactggggccagggcacactggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagagagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaa

SEQ ID NO: 76 - protein, 216 aa, synthetic construct
QSVLTQPPSASGTPGQRVTISCSGSSSNIGDNYVSWYQQLPGTAPKLLIYRDSQRPSGVPDRFSGSKSGTSASLAISGLRSEDEADYYCQSYDSSLSGSVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 77 - DNA, 648 bp, synthetic construct
  source 1..648: mol_type = other DNA
caatctgttctgacacagcctcctagcgcctctggcacacctggacagagagtgaccatcagctgtagcggcagcagcagcaacatcggcgacaactacgtgtcctggtatcagcagctgcctggcacagcccctaaactgctgatctaccgggatagccagaggccaagcggcgtgccagatagattttccggcagcaagagcggcacatctgccagcctggctatcagcggactgagatctgaggacgaggccgactactactgccagagctacgatagcagcctgagcggctctgtgtttggcggaggtaccaagctgaccgtgctgggccaacctaaagccaatcctaccgtgacactgttccctccaagcagcgaagaactgcaggccaacaaggccacactcgtgtgcctgatcagcgacttttatcctggcgccgtgaccgtggcctggaaggctgatggatctcctgtgaaagccggcgtggaaaccaccaagcctagcaagcagagcaacaacaaatacgccgccagcagctacctgagcctgacacctgagcagtggaagtcccacagatcctacagctgccaagtgacccacgagggcagcaccgtggaaaagacagtggcccctaccgagtgcagc

SEQ ID NO: 78 - protein, 448 aa, synthetic construct
EVQLVQSGAEVKKPGASVKVSCKASGYTFTSQYMHWVRQAPGQGLEWIGIINPSGVHTSYAQKFQGRVTLTRDTSTSTLYMELSSLRSEDTAVYYCARGSPKGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - DNA, 1344 bp, synthetic construct
  source 1..1344: mol_type = other DNA
gaagttcagctggttcagtctggcgccgaagtgaagaaacctggcgcctctgtgaaggtgtcctgcaaggccagcggctacacctttaccagccagtacatgcactgggtccgacaggctccaggacagggactcgagtggatcggcatcatcaatcctagcggcgtgcacaccagctacgcccagaaattccagggcagagtgaccctgaccagagacaccagcaccagcacactgtacatggaactgagcagcctgcggagcgaggataccgccgtgtactattgtgccagaggctctcctaagggcgccttcgattattggggccagggcacactggtcaccgtgtcctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaa

SEQ ID NO: 80 - protein, 217 aa, synthetic construct
EIVMMQSPGTLSLSPGERATLSCRASQSVRSNYLAWYQQKPGQAPRLLIYGASSRATGIPDRFSGSGSGTDFTLTISRLEPEDFAVYYCQQYGSSPPRFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - DNA, 651 bp, synthetic construct
  source 1..651: mol_type = other DNA
gagatcgtcatgatgcagagccccggcacactgtcactgtctccaggcgaaagagccacactgagctgtagagcctctcagagcgtgcggagcaattacctggcctggtatcagcagaagcccggacaggctcctcggctgttgatctatggcgcctctagcagagccactggcatccccgatagattttctggcagcggctccggcaccgacttcaccctgacaatcagcagactggaacccgaggacttcgccgtgtactactgtcagcagtacggcagcagccctcctcggttcacatttggccctggtaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgc

SEQ ID NO: 82 - protein, 447 aa, synthetic construct
QVQEVQLLESGGGLVQPGGSLRLSCAASGFTFSSYVMTWARQAPGKGLEWVSAISGTGYTYYADSVKGRFTVSRDNSKNTLFLQMSSLRAEDTAVYYCAITMAPVVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 455 aa, synthetic construct
QVQLVESGAEVKKPGESLKISCKGSGYTFTRYWIGWVRQMPGKGLEWMGIIYPGDSDTRYSPSFQGHVTISADKSISTAYLQWNSLKASDTAMYYCARLPQYCSNGVCQRWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - DNA, 1341 bp, synthetic construct
  source 1..1341: mol_type = other DNA
caagtgcaggaagtgcagctgctggaatctggcggaggactggttcaaccaggcggctctctgagactgtcttgtgccgccagcggcttcaccttcagcagctacgttatgacctgggccagacaggcccctggcaaaggacttgaatgggtgtccgccatcagcggcacaggctacacctactacgccgactctgtgaagggcagattcaccgtgtccagagacaacagcaagaataccctgttcctgcagatgagcagcctgagagccgaggataccgccgtgtactactgcgccattacaatggcccctgtcgtgtggggccagggcacaacagtgacagtcagcagcgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaa

SEQ ID NO: 85 - DNA, 1365 bp, synthetic construct
  source 1..1365: mol_type = other DNA
caagtgcagctggttgaatctggcgccgaagtgaagaagcctggcgagagcctgaagatcagctgcaaaggcagcggctacaccttcaccagatactggatcggctgggtccgacagatgcctggcaaaggccttgagtggatgggcatcatctaccccggcgacagcgataccagatacagccctagctttcagggccacgtgaccatcagcgccgacaagtctatcagcaccgcctacctgcagtggaacagcctgaaggccagcgacaccgccatgtactactgtgccagactgccccagtactgcagcaatggcgtttgccagcgttggttcgatccttggggacagggcacactggtcaccgttagcagcgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaa

SEQ ID NO: 86 - protein, 216 aa, synthetic construct
SYELTQPPSVSVSPGQTARITCSGDALPKRYAYWYQQKSGQAPILVIYEDKKRPSGIPERLSGSKSGTVATLTISGAQVEDEADYYCYSTDSSGNHAVFGGGTQLTVLGQPKAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 214 aa, synthetic construct
EIVLTQSPSSVSASVGDRVTITCRASQGISSWLAWYQQKPGKAPKLLIYAASSLQSGVPSRFSGSGSGTDFTLTISSLQPEDFATYYCQQGNSFPYTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - DNA, 648 bp, synthetic construct
  source 1..648: mol_type = other DNA
agctatgaactgacacagcctccaagcgtgtccgtgtctcctggacagaccgccagaatcacatgtagcggagatgccctgcctaagagatacgcctactggtatcagcagaagtccggacaggcccctatcctggtcatctacgaggacaagaagcggccctctggcatccctgagagactgagcggaagcaagtctggcacagtggccacactgacaatctctggcgcccaggtggaagatgaggccgactactactgctacagcaccgacagctctggcaaccacgctgtgtttggcggaggtacccaactgaccgtgctgggccaacctaaagccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgc

SEQ ID NO: 89 - DNA, 642 bp, synthetic construct
  source 1..642: mol_type = other DNA
gaaattgtgctgacacagagccccagcagcgtgtcagcctctgtgggagacagagtgaccatcacctgtagagccagccagggcatctcttcttggctggcctggtatcagcagaagcctggcaaggcccctaagctgctgatctatgccgctagctctctgcagtctggcgtgcccagcagattttctggcagcggctctggcaccgacttcaccctgaccatatctagcctgcagcctgaggacttcgccacctactattgccagcagggcaacagcttcccctacacctttggccagggtaccaacctggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgc

SEQ ID NO: 90 - protein, 448 aa, synthetic construct
EVQLVQSGAEVKKPGASVKVSCKASGYTFTSQYMHWVRQAPGQGLEWIGIINPSGVHTSYAQKFQGRVTLTRDTSTSTLYMELSSLRSEDTAVYYCARGSPKGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMFEALHNHYTQKSLSLSPGK

SEQ ID NO: 91 - DNA, 1344 bp, synthetic construct
  source 1..1344: mol_type = other DNA
gaagttcagctggttcagtctggcgccgaagtgaagaaacctggcgcctctgtgaaggtgtcctgcaaggccagcggctacacctttaccagccagtacatgcactgggtccgacaggctccaggacagggactcgagtggatcggcatcatcaatcctagcggcgtgcacaccagctacgcccagaaattccagggcagagtgaccctgaccagagacaccagcaccagcacactgtacatggaactgagcagcctgcggagcgaggataccgccgtgtactattgtgccagaggctctcctaagggcgccttcgattattggggccagggcacactggtcaccgtgtcctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaagtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggatgaactgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggaaaa

SEQ ID NO: 92 - protein, 447 aa, synthetic construct
QVQEVQLLESGGGLVQPGGSLRLSCAASGFTFSSYVMTWARQAPGKGLEWVSAISGTGYTYYADSVKGRFTVSRDNSKNTLFLQMSSLRAEDTAVYYCAITMAPVVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMFEALHNHYTQKSLSLSPGK

SEQ ID NO: 93 - DNA, 1341 bp, synthetic construct
  source 1..1341: mol_type = other DNA
caagtgcaggaagtgcagctgctggaatctggcggaggactggttcaaccaggcggctctctgagactgtcttgtgccgccagcggcttcaccttcagcagctacgttatgacctgggccagacaggcccctggcaaaggacttgaatgggtgtccgccatcagcggcacaggctacacctactacgccgactctgtgaagggcagattcaccgtgtccagagacaacagcaagaataccctgttcctgcagatgagcagcctgagagccgaggataccgccgtgtactactgcgccattacaatggcccctgtcgtgtggggccagggcacaacagtgacagtcagcagcgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggatgaactgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggaaaa

SEQ ID NO: 94 - protein, 455 aa, synthetic construct
QVQLVESGAEVKKPGESLKISCKGSGYTFTRYWIGWVRQMPGKGLEWMGIIYPGDSDTRYSPSFQGHVTISADKSISTAYLQWNSLKASDTAMYYCARLPQYCSNGVCQRWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMFEALHNHYTQKSLSLSPGK

SEQ ID NO: 95 - DNA, 1365 bp, synthetic construct
  source 1..1365: mol_type = other DNA
caagtgcagctggttgaatctggcgccgaagtgaagaagcctggcgagagcctgaagatcagctgcaaaggcagcggctacaccttcaccagatactggatcggctgggtccgacagatgcctggcaaaggccttgagtggatgggcatcatctaccccggcgacagcgataccagatacagccctagctttcagggccacgtgaccatcagcgccgacaagtctatcagcaccgcctacctgcagtggaacagcctgaaggccagcgacaccgccatgtactactgtgccagactgccccagtactgcagcaatggcgtttgccagcgttggttcgatccttggggacagggcacactggtcaccgttagcagcgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggatgaactgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggaaaa

SEQ ID NO: 96 - protein, 19 aa, synthetic construct
HEALHNHYTQKSLSLSPGK

SEQ ID NO: 97 - DNA, 57 bp, synthetic construct
  source 1..57: mol_type = other DNA
cacgaagcgctgcacaatcattatactcagaaaagcctgagcttgagcccgggaaaa

SEQ ID NO: 98 - protein, 19 aa, synthetic construct
FEALHNHYTQKSLSLSPGK

SEQ ID NO: 99 - DNA, 57 bp, synthetic construct
  source 1..57: mol_type = other DNA
ttcgaggcgctgcacaatcattatactcagaaaagcctgagcttgagcccgggaaaa

SEQ ID NO: 100 - protein, 19 aa, synthetic construct
FEALHNHYTQESLSLSPGK

SEQ ID NO: 101 - DNA, 57 bp, synthetic construct
  source 1..57: mol_type = other DNA
ttcgaagcgctgcacaatcattatactcaggagagcctgagcttgagcccgggaaaa

SEQ ID NO: 102 - protein, 19 aa, synthetic construct
FEALHNHYTQKKLSLSPGK

SEQ ID NO: 103 - DNA, 57 bp, synthetic construct
  source 1..57: mol_type = other DNA
ttcgaagcgctgcacaatcattatactcagaaaaagctgagcttgagcccgggaaaa

SEQ ID NO: 104 - protein, 1065 aa, synthetic construct
ETGSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDRAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - DNA, 3195 bp, synthetic construct
  source 1..3195: mol_type = other DNA
gaaaccggttccaccattgaggaacaggccaagtactttttggacaagtttaaccacgaagccgaagacctgttctatcaaagttcacttgcttcttggaattataacaccaatattactgaagagaatgtccaaaacatgaacaacgccggcgacaagtggagcgccttcctgaaagagcagagcacaacagcccagatgtaccctctgcaagagatccagaacctgaccgtgaagctccagctgcaggccctccagcagaatggaagctctgtgctgagcgaggacaagagcaagcggctgaacaccatcctgaataccatgagcaccatctacagcaccggcaaagtgtgcaaccccgacaatccccaagagtgcctgctgctggaacccggcctgaatgagatcatggccaacagcctggactacaacgagagactgtgggcctgggagtcttggagaagcgaagtgggaaagcagctgcggcccctgtacgaggaatacgtggtgctgaagaacgagatggccagagccaaccactacgaggactacggcgactattggagaggcgactacgaagtgaatggcgtggacggctacgactacagcagaggccagctgatcgaggacgtggaacacaccttcgaggaaatcaagcctctgtacgagcatctgcacgcctacgtgcgggccaagctgatgaatgcttaccccagctacatcagccccatcggctgtctgcctgctcatctgctgggagacatgtggggcagattctggaccaacctgtacagcctgacagtgcccttcggccagaaacctaacatcgacgtgaccgacgctatggtggatcaggcttgggatgcccagcggatcttcaaagaggccgagaagttcttcgtgtccgtgggcctgcctaatatgacccaaggcttctgggagtactccatgctgacagaccccggcaacgtgcagaaagccgtgtgtcatcctaccgcctgggatctcggcaagggcgacttcagaatcctgatgtgcaccaaagtgacgatggacgacttcctgacagcccaccacgagatgggccacatccagtacgatatggcctacgccgctcagcccttcctgctgagaaatggcgccaatgagggcttccatgaggccgtgggagagatcatgagcctgtctgccgccacacctaagcacctgaagtctatcggactgctgagccccgacttccaagaggacaacgagacagagatcaacttcctgctcaagcaggccctgaccatcgtgggcacactgccctttacctacatgctggaaaagtggcggtggatggtctttaagggcgagatccccaaggaccagtggatgaagaaatggtgggagatgaagcgcgagatcgtgggcgttgtggaacctgtgcctcacgacgagacatactgcgatcctgccagcctgtttcacgtgtccaacgactactccttcatccggtactacacccggacactgtaccagttccagtttcaagaggctctgtgccaggccgccaagcacgaaggacctctgcacaagtgcgacatcagcaactctacagaggccggacagaaactgttcaacatgctgcggctgggcaagagcgagccttggacactggctctggaaaatgtcgtgggcgccaagaacatgaatgtgcggccactgctgaactacttcgagcccctgttcacctggctgaaggaccagaacaagaacagcttcgtcggctggtcgaccgattggagcccttatgccgatcagagcatcaaagtgcggatcagcctgaagtctgccctgggcgatagagcctacgagtggaacgacaacgagatgtacctgttccggtccagcgtggcatatgctatgcggcagtacttcctgaaagtgaagaaccagatgatcctgttcggcgaagaggacgtgcgcgtggccaatctgaagcccagaatcagcttcaacttcttcgtgacagcccctaagaacgtgtccgatatcatccccagaaccgaggtggaaaaggccatcagaatgagcagaagccggatcaacgacgccttccggctgaacgacaatagcctggaatttctgggcatccagcctacactgggccctccaaatcagcctcctgttagcggaggaggaggatctggaggaggaggatccgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgagctcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaa

SEQ ID NO: 106 - protein, 842 aa, synthetic construct
ETGSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDRAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - DNA, 2526 bp, synthetic construct
  source 1..2526: mol_type = other DNA
gaaaccggttccaccattgaggaacaggccaagtactttttggacaagtttaaccacgaagccgaagacctgttctatcaaagttcacttgcttcttggaattataacaccaatattactgaagagaatgtccaaaacatgaacaacgccggcgacaagtggagcgccttcctgaaagagcagagcacaacagcccagatgtaccctctgcaagagatccagaacctgaccgtgaagctccagctgcaggccctccagcagaatggaagctctgtgctgagcgaggacaagagcaagcggctgaacaccatcctgaataccatgagcaccatctacagcaccggcaaagtgtgcaaccccgacaatccccaagagtgcctgctgctggaacccggcctgaatgagatcatggccaacagcctggactacaacgagagactgtgggcctgggagtcttggagaagcgaagtgggaaagcagctgcggcccctgtacgaggaatacgtggtgctgaagaacgagatggccagagccaaccactacgaggactacggcgactattggagaggcgactacgaagtgaatggcgtggacggctacgactacagcagaggccagctgatcgaggacgtggaacacaccttcgaggaaatcaagcctctgtacgagcatctgcacgcctacgtgcgggccaagctgatgaatgcttaccccagctacatcagccccatcggctgtctgcctgctcatctgctgggagacatgtggggcagattctggaccaacctgtacagcctgacagtgcccttcggccagaaacctaacatcgacgtgaccgacgctatggtggatcaggcttgggatgcccagcggatcttcaaagaggccgagaagttcttcgtgtccgtgggcctgcctaatatgacccaaggcttctgggagtactccatgctgacagaccccggcaacgtgcagaaagccgtgtgtcatcctaccgcctgggatctcggcaagggcgacttcagaatcctgatgtgcaccaaagtgacgatggacgacttcctgacagcccaccacgagatgggccacatccagtacgatatggcctacgccgctcagcccttcctgctgagaaatggcgccaatgagggcttccatgaggccgtgggagagatcatgagcctgtctgccgccacacctaagcacctgaagtctatcggactgctgagccccgacttccaagaggacaacgagacagagatcaacttcctgctcaagcaggccctgaccatcgtgggcacactgccctttacctacatgctggaaaagtggcggtggatggtctttaagggcgagatccccaaggaccagtggatgaagaaatggtgggagatgaagcgcgagatcgtgggcgttgtggaacctgtgcctcacgacgagacatactgcgatcctgccagcctgtttcacgtgtccaacgactactccttcatccggtactacacccggacactgtaccagttccagtttcaagaggctctgtgccaggccgccaagcacgaaggacctctgcacaagtgcgacatcagcaactctacagaggccggacagaaactgttcaacatgctgcggctgggcaagagcgagccttggacactggctctggaaaatgtcgtgggcgccaagaacatgaatgtgcggccactgctgaactacttcgagcccctgttcacctggctgaaggaccagaacaagaacagcttcgtcggctggtcgaccgattggagcccttatgccgatcagagcatcaaagtgcggatcagcctgaagtctgccctgggcgatagagcctacgagtggaacgacaacgagatgtacctgttccggtccagcgtggcatatgctatgcggcagtacttcctgaaagtgaagaaccagatgatcctgttcggcgaagaggacgtgcgcgtggccaatctgaagcccagaatcagcttcaacttcttcgtgacagcccctaagaacgtgtccgatatcatccccagaaccgaggtggaaaaggccatcagaatgagcagaagccggatcaacgacgccttccggctgaacgacaatagcctggaatttctgggcatccagcctacactgggccctccaaatcagcctcctgttagcggaggaggaggatctggaggaggaggctcgaggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgc

SEQ ID NO: 108 - protein, 1065 aa, synthetic construct
ETGSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDRAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMFEALHNHYTQKSLSLSPGK

SEQ ID NO: 109 - DNA, 3195 bp, synthetic construct
  source 1..3195: mol_type = other DNA
gaaaccggttccaccattgaggaacaggccaagtactttttggacaagtttaaccacgaagccgaagacctgttctatcaaagttcacttgcttcttggaattataacaccaatattactgaagagaatgtccaaaacatgaacaacgccggcgacaagtggagcgccttcctgaaagagcagagcacaacagcccagatgtaccctctgcaagagatccagaacctgaccgtgaagctccagctgcaggccctccagcagaatggaagctctgtgctgagcgaggacaagagcaagcggctgaacaccatcctgaataccatgagcaccatctacagcaccggcaaagtgtgcaaccccgacaatccccaagagtgcctgctgctggaacccggcctgaatgagatcatggccaacagcctggactacaacgagagactgtgggcctgggagtcttggagaagcgaagtgggaaagcagctgcggcccctgtacgaggaatacgtggtgctgaagaacgagatggccagagccaaccactacgaggactacggcgactattggagaggcgactacgaagtgaatggcgtggacggctacgactacagcagaggccagctgatcgaggacgtggaacacaccttcgaggaaatcaagcctctgtacgagcatctgcacgcctacgtgcgggccaagctgatgaatgcttaccccagctacatcagccccatcggctgtctgcctgctcatctgctgggagacatgtggggcagattctggaccaacctgtacagcctgacagtgcccttcggccagaaacctaacatcgacgtgaccgacgctatggtggatcaggcttgggatgcccagcggatcttcaaagaggccgagaagttcttcgtgtccgtgggcctgcctaatatgacccaaggcttctgggagtactccatgctgacagaccccggcaacgtgcagaaagccgtgtgtcatcctaccgcctgggatctcggcaagggcgacttcagaatcctgatgtgcaccaaagtgacgatggacgacttcctgacagcccaccacgagatgggccacatccagtacgatatggcctacgccgctcagcccttcctgctgagaaatggcgccaatgagggcttccatgaggccgtgggagagatcatgagcctgtctgccgccacacctaagcacctgaagtctatcggactgctgagccccgacttccaagaggacaacgagacagagatcaacttcctgctcaagcaggccctgaccatcgtgggcacactgccctttacctacatgctggaaaagtggcggtggatggtctttaagggcgagatccccaaggaccagtggatgaagaaatggtgggagatgaagcgcgagatcgtgggcgttgtggaacctgtgcctcacgacgagacatactgcgatcctgccagcctgtttcacgtgtccaacgactactccttcatccggtactacacccggacactgtaccagttccagtttcaagaggctctgtgccaggccgccaagcacgaaggacctctgcacaagtgcgacatcagcaactctacagaggccggacagaaactgttcaacatgctgcggctgggcaagagcgagccttggacactggctctggaaaatgtcgtgggcgccaagaacatgaatgtgcggccactgctgaactacttcgagcccctgttcacctggctgaaggaccagaacaagaacagcttcgtcggctggtcgaccgattggagcccttatgccgatcagagcatcaaagtgcggatcagcctgaagtctgccctgggcgatagagcctacgagtggaacgacaacgagatgtacctgttccggtccagcgtggcatatgctatgcggcagtacttcctgaaagtgaagaaccagatgatcctgttcggcgaagaggacgtgcgcgtggccaatctgaagcccagaatcagcttcaacttcttcgtgacagcccctaagaacgtgtccgatatcatccccagaaccgaggtggaaaaggccatcagaatgagcagaagccggatcaacgacgccttccggctgaacgacaatagcctggaatttctgggcatccagcctacactgggccctccaaatcagcctcctgttagcggaggaggaggatctggaggaggaggatccgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgagctcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggaaaa

SEQ ID NO: 110 - protein, 447 aa, synthetic construct
EVQLQQSGAEVVKPGASVKLSCKASGFNIKDTFIHWVKQAPGQGLEWIGRIDPANTNTKYDPKFQGKATITTDTSSNTAYMELSSLRSEDTAVYYCVRGLYTYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMFEALHNHYTQKSLSLSPG

SEQ ID NO: 111 - DNA, 1341 bp, synthetic construct
  source 1..1341: mol_type = other DNA
gaagtgcagctgcaacagtctggcgccgaggttgtgaaacctggcgcctctgtgaagctgagctgtaaagccagcggcttcaacatcaaggacaccttcatccactgggtcaagcaggcccctggacagggactcgaatggatcggcagaatcgaccccgccaacaccaacactaagtacgaccccaagttccagggcaaagccaccatcaccaccgacaccagcagcaacaccgcctacatggaactgagcagcctgagaagcgaggacaccgccgtgtactattgtgtgcggggcctgtacacctactacttcgattattggggccagggtaccctggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggcacccagacctatatctgcaatgtcaatcataaacccagtaacacaaaggtagacaagagagtcgaacccaaatcttgcgacaaaactcacacatgccctccttgccccgcccccgaagccgcggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccggga

SEQ ID NO: 112 - protein, 447 aa, synthetic construct
QVQLVQSGAEVKKPGASVKVSCKASGFNIKDTHMHWVRQAPGQRLEWIGRIDPANGNTEYDQKFQGRVTITVDTSASTAYMELSSLRSEDTAVYYCARWGTNVYFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMFEALHNHYTQKSLSLSPG

SEQ ID NO: 113 - DNA, 1341 bp, synthetic construct
  source 1..1341: mol_type = other DNA
caggttcagctggttcagtctggcgccgaagtgaagaaacctggcgcctctgtgaaggtgtcctgcaaggccagcggcttcaacatcaaggacacccacatgcactgggtccgacaggctccaggacagagactggaatggatcggcagaatcgaccccgccaacggcaacaccgagtacgaccagaaattccagggcagagtgaccatcaccgtggacacatctgccagcaccgcctacatggaactgagcagcctgagaagcgaggacaccgccgtgtactactgtgccagatggggcaccaacgtgtacttcgcctattggggccagggtaccctggtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggcacccagacctatatctgcaatgtcaatcataaacccagtaacacaaaggtagacaagagagtcgaacccaaatcttgcgacaaaactcacacatgccctccttgccccgcccccgaagccgcggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccggga

SEQ ID NO: 114 - protein, 443 aa, synthetic construct
EVQLQESGPSLVKPSQTLSLTCSVTGDSITSGYWNWIRQVPGNKLEYMGFINYSGNTYYNPSLRSRISITRDTSKNQYFLHLNSVTTEDTATYYCARANWDVFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 115 - DNA, 1329 bp, synthetic construct
  source 1..1329: mol_type = other DNA
gaggtgcagttgcaggaatctggtcccagtctagttaagcccagccagacactgagcctgacatgtagtgtgaccggtgacagcattacaagcggctactggaactggatcagacaggtgccaggaaacaaactcgaatacatggggttcatcaattacagtggcaatacttactataatcccagcctgagaagcagaatttctataaccagagacaccagcaaaaaccagtactttctgcacctgaacagcgtaacaacggaagacaccgccacctattactgcgccagggccaactgggatgtgttcgcatactggggccagggcactctggtgacggtgagcgccgcgtcgacaaaaggtcccagcgtgtttcccctggctccttgtagcagaagcacgtcagaaagtacagctgccctgggatgcctggtgaaagattattttcccgagcccgttaccgtttcctggaatagtggcgccctgacctcgggcgtgcatacttttcccgctgttctgcagagcagcggcctgtatagcctgagcagtgtggtgactgttccgtctagcagcctgggtaccaagacttatacatgcaacgtggaccataagcctagcaatactaaggtagataagcgagtggagagcaagtacggccccccctgccctagctgtcccgcacctgagtttctaggcgggcccagcgtgtttctgttccccccgaagcctaaagataccctgatgatcagcagaacacccgaagtgacctgcgtggttgtggacgtcagccaggaagatcctgaggtgcagttcaattggtacgtggacggcgtggaggtgcacaatgctaagacaaagccccgcgaagagcaattcaacagcacatatagagtagtatctgtgctgaccgtgctgcaccaggattggctgaacggcaaggaatacaaatgtaaagtgagcaacaagggcttacccagtagcatcgagaagaccattagcaaagcaaaaggtcaaccccgggagccccaggtgtacaccctgcctccgagccaagaggaaatgacaaagaatcaggtgagcctgacctgcctggtgaagggattttaccccagcgatattgctgtggagtgggagagcaatgggcaaccagagaataactacaaaacgaccccaccagtattagactcggacggatctttcttcttgtattcgcggctgactgtggacaagagcagatggcaagagggcaatgttttcagctgtagcgttatgcacgaagcgctgcacaatcattatactcagaaaagcctgagcttgagcttaggaaaa

SEQ ID NO: 116 - protein, 443 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPEFLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 117 - DNA, 1329 bp, synthetic construct
  source 1..1329: mol_type = other DNA
gaggtgcagcttcaggagtcaggacctagcctcgtgaaaccttctcagactctgtccctcacatgttctgtcactggcgactccatcaccagtggttactggaactggatccggcaattcccagggaataaacttgagtacatgggtttcataaattacagtggtaacacttactacaatccatctctcagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgcagcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccagagtttttgggaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 118 - protein, 443 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPELLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 119 - DNA, 1329 bp, synthetic construct
  source 1..1329: mol_type = other DNA
gaggtgcagcttcaggagtcaggacctagcctcgtgaaaccttctcagactctgtccctcacatgttctgtcactggcgactccatcaccagtggttactggaactggatccggcaattcccagggaataaacttgagtacatgggtttcataaattacagtggtaacacttactacaatccatctctcagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgcagcctccaccaagggcccatcggtcttccccctggcgccctgctccaggagcacctccgagagcacagccgccctgggctgcctggtcaaggactacttccccgaaccggtgacggtgtcgtggaactcaggcgctctgaccagcggcgtgcacaccttcccagctgtcctacagtcctcaggactctactccctcagcagcgtggtgaccgtgccctccagcaacttcggcacccagacctacacctgcaacgtagatcacaagcccagcaacaccaaggtggacaagacagttgagcgcaaatgttgtgtcgagtgcccaccgtgcccagcaccagagcttttgggaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgttgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 120 - protein, 443 aa, synthetic construct
EVQLQESGPSLVKPSQTLSLTCSVTGDSITSGYWNWIRQVPGNKLEYMGFINYSGNTYYNPSLRSRISITRDTSKNQYFLHLNSVTTEDTATYYCARANWDVFAYWGQGTLVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPEFEGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSLGK

SEQ ID NO: 121 - DNA, 1329 bp, synthetic construct
  source 1..1329: mol_type = other DNA
gaggtgcagttgcaggaatctggtcccagtctagttaagcccagccagacactgagcctgacatgtagtgtgaccggtgacagcattacaagcggctactggaactggatcagacaggtgccaggaaacaaactcgaatacatggggttcatcaattacagtggcaatacttactataatcccagcctgagaagcagaatttctataaccagagacaccagcaaaaaccagtactttctgcacctgaacagcgtaacaacggaagacaccgccacctattactgcgccagggccaactgggatgtgttcgcatactggggccagggcactctggtgacggtgagcgccgcgtcgacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggtacccagacctatacatgcaatgtcgatcataaacccagtaacacaaaggtagacaagacagtcgaaagaaaatgctgtgtggagtgccctccttgccccgcccccgagtttgagggggggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcttaggaaaa

SEQ ID NO: 122 - protein, 445 aa, synthetic construct
EVQLQESGPSLVKPSQTLSLTCSVTGDSITSGYWNWIRQVPGNKLEYMGFINYSGNTYYNPSLRSRISITRDTSKNQYFLHLNSVTTEDTATYYCARANWDVFAYWGQGTLVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSLGK

SEQ ID NO: 123 - DNA, 1335 bp, synthetic construct
  source 1..1335: mol_type = other DNA
gaggtgcagttgcaggaatctggtcccagtctagttaagcccagccagacactgagcctgacatgtagtgtgaccggtgacagcattacaagcggctactggaactggatcagacaggtgccaggaaacaaactcgaatacatggggttcatcaattacagtggcaatacttactataatcccagcctgagaagcagaatttctataaccagagacaccagcaaaaaccagtactttctgcacctgaacagcgtaacaacggaagacaccgccacctattactgcgccagggccaactgggatgtgttcgcatactggggccagggcactctggtgacggtgagcgccgctagcacaaaagggcccagcgtctttccactcgccccctgttcaaggagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgcctagcagcaattttggtacccagacctatacatgcaatgtcgatcataaacccagtaacacaaaggtcgacaagacagtcgaacccaagagctgcgacaagacccacacctgccctccttgccctgcccccccagtggccgggccttcggtttttctttttccacctaaaccgaaagacaccctgatgatctcgaggacaccagaggtcacctgtgtcgtcgtggacgtcagtcacgaggatccggaggtgcaattcaactggtatgttgatggtgtggaagtgcataatgcaaaaactaagccccgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcttaggaaaa

SEQ ID NO: 124 - protein, 443 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSSASTKGPSVFPLAPSSRSTSGGTAALGCLVKDYFPEPVTVSWNSGALTSGVHTFPAVLQSSGLYSLSSVVTVPSSSLGTQTYICNVNHKPSNTKVDKKVERKCCVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - DNA, 1329 bp, synthetic construct
  source 1..1329: mol_type = other DNA
gaggttcagctgcaagagtctggccctagcctggtcaagcctagccagacactgagcctgacctgtagcgtgaccggcgatagcatcacaagcggctactggaactggatcagacagttccccggcaacaaacttgagtacatgggcttcatcaactacagcggcaacacctactacaaccccagcctgcggagccggatcagcatcaccagagacaccagcaagaaccagtacttcctgcacctgaacagcgtgaccaccgaggataccgccacctactattgcgccagagccaactgggacgtgttcgcctattggggccagggaacactcgtgaccgttagctccgctagcacaaagggccctagcgtctttccactcgccccctcctccaggtctacatctggtggaacagccgctctgggctgtctggtcaaggactactttcccgagcctgtgaccgtgtcctggaatagcggagcactgacaagcggcgtgcacacctttccagccgtgctgcaatctagcggcctgtactctctgagcagcgtggtcacagtgcctagctctagcctcggtacccagacctacatctgcaatgtgaatcacaagcccagcaacaccaaggtcgacaagaaggtggaaagaaaatgctgtgtggagtgtcctccatgtcctgctccagagctcctcggaggcccttccgtgtttctgttccctccaaagcctaaggacaccctgatgatctcgaggacccctgaggtcacctgtgtggtggttgacgtcagtcacgaggatcctgaagtgaagttcaattggtacgtggacggcgtggaagtgcacaacgccaagacaaaaccccgggaggaacagtacaacagcacctacagagtggtgtccgtgctgacagtgctgcaccaggattggctgaacggcaaagagtacaagtgcaaggtgtccaacaaagctctgcccgctcctatcgagaaaacgatatccaaggccaagggccagcctcgggaaccccaagtgtataccttgcctcctagccgggacgagctgaccaagaatcaggtgtccctgacctgcctcgtgaaaggcttctaccccagcgacattgccgtggaatgggagagcaatggccagcctgagaacaactacaagacaacccctcctgtgctggacagcgacggctcattcttcctgtacagcaagctgaccgtggacaagtcgcgatggcagcagggcaacgtgttctcctgttccgtgatgcacgaagcgctgcacaaccactacacccagaagtccctgtctctgagccccggcaag

SEQ ID NO: 126 - protein, 446 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMFEALHNHYTQKSLSLSPGK

SEQ ID NO: 127 - DNA, 1338 bp, synthetic construct
  source 1..1338: mol_type = other DNA
gaggtgcagcttcaggagtcaggacctagcctcgtgaaaccttctcagactctgtccctcacatgttctgtcactggcgactccatcaccagtggttactggaactggatccggcaattcccagggaataaacttgagtacatgggtttcataaattacagtggtaacacttactacaatccatctctcagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgtttgaggctctgcacaaccactacacgcagaagagcctctccctgtctccgggtaaa

SEQ ID NO: 128 - protein, 446 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHGALHNHYTQKSLSLSPGK

SEQ ID NO: 129 - DNA, 1338 bp, synthetic construct
  source 1..1338: mol_type = other DNA
gaggtgcagcttcaggagtcaggacctagcctcgtgaaaccttctcagactctgtccctcacatgttctgtcactggcgactccatcaccagtggttactggaactggatccggcaattcccagggaataaacttgagtacatgggtttcataaattacagtggtaacacttactacaatccatctctcagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatggggctctgcacaaccactacacgcagaagagcctctccctgtctccgggtaaa

SEQ ID NO: 130 - protein, 446 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMQEALHNHYTQKSLSLSPGK

SEQ ID NO: 131 - DNA, 1338 bp, synthetic construct
  source 1..1338: mol_type = other DNA
gaggtgcagcttcaggagtcaggacctagcctcgtgaaaccttctcagactctgtccctcacatgttctgtcactggcgactccatcaccagtggttactggaactggatccggcaattcccagggaataaacttgagtacatgggtttcataaattacagtggtaacacttactacaatccatctctcagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcaggaggctctgcacaaccactacacgcagaagagcctctccctgtctccgggtaaa

SEQ ID NO: 132 - protein, 446 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMEEALHNHYTQKSLSLSPGK

SEQ ID NO: 133 - DNA, 1338 bp, synthetic construct
  source 1..1338: mol_type = other DNA
gaggtgcagcttcaggagtcaggacctagcctcgtgaaaccttctcagactctgtccctcacatgttctgtcactggcgactccatcaccagtggttactggaactggatccggcaattcccagggaataaacttgagtacatgggtttcataaattacagtggtaacacttactacaatccatctctcagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatggaagaggctctgcacaaccactacacgcagaagagcctctccctgtctccgggtaaa

SEQ ID NO: 134 - protein, 446 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNAYTQKSLSLSPGK

SEQ ID NO: 135 - DNA, 1338 bp, synthetic construct
  source 1..1338: mol_type = other DNA
gaggtgcagcttcaggagtcaggacctagcctcgtgaaaccttctcagactctgtccctcacatgttctgtcactggcgactccatcaccagtggttactggaactggatccggcaattcccagggaataaacttgagtacatgggtttcataaattacagtggtaacacttactacaatccatctctcagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaacgcctacacgcagaagagcctctccctgtctccgggtaaa

SEQ ID NO: 136 - protein, 446 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSAASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - DNA, 1338 bp, synthetic construct
  source 1..1338: mol_type = other DNA
gaggtgcagcttcaggagtcaggacctagcctcgtgaaaccttctcagactctgtccctcacatgttctgtcactggcgactccatcaccagtggttactggaactggatccggcaattcccagggaataaacttgagtacatgggtttcataaattacagtggtaacacttactacaatccatctctcagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgcagcctccaccaagggcccatcggtcttccccctggcaccctgctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 138 - protein, 443 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSAASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKKVESKYGPPCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - DNA, 1329 bp, synthetic construct
  source 1..1329: mol_type = other DNA
gaggtgcagcttcaggagtcaggacctagcctcgtgaaaccttctcagactctgtccctcacatgttctgtcactggcgactccatcaccagtggttactggaactggatccggcaattcccagggaataaacttgagtacatgggtttcataaattacagtggtaacacttactacaatccatctctcagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgcagcctccaccaagggcccatcggtcttccccctggcaccctgctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagagcaagtacggccccccctgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 140 - protein, 446 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREENSTSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - DNA, 1338 bp, synthetic construct
  source 1..1338: mol_type = other DNA
gaggtgcagcttcaggagtcaggacctagcctcgtgaaaccttctcagactctgtccctcacatgttctgtcactggcgactccatcaccagtggttactggaactggatccggcaattcccagggaataaacttgagtacatgggtttcataaattacagtggtaacacttactacaatccatctctcagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagaacagcacgagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 142 - protein, 446 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQNSSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - DNA, 1338 bp, synthetic construct
  source 1..1338: mol_type = other DNA
gaggtgcagcttcaggagtcaggacctagcctcgtgaaaccttctcagactctgtccctcacatgttctgtcactggcgactccatcaccagtggttactggaactggatccggcaattcccagggaataaacttgagtacatgggtttcataaattacagtggtaacacttactacaatccatctctcagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagaacagcagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 144 - protein, 446 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHAAPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - DNA, 1338 bp, synthetic construct
  source 1..1338: mol_type = other DNA
gaggtgcagcttcaggagtcaggacctagcctcgtgaaaccttctcagactctgtccctcacatgttctgtcactggcgactccatcaccagtggttactggaactggatccggcaattcccagggaataaacttgagtacatgggtttcataaattacagtggtaacacttactacaatccatctctcagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgcagcccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 146 - protein, 446 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMSEALHNHYTQKSLSLSPGK

SEQ ID NO: 147 - DNA, 1338 bp, synthetic construct
  source 1..1338: mol_type = other DNA
gaggtgcagcttcaggagtcaggacctagcctcgtgaaaccttctcagactctgtccctcacatgttctgtcactggcgactccatcaccagtggttactggaactggatccggcaattcccagggaataaacttgagtacatgggtttcataaattacagtggtaacacttactacaatccatctctcagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgtcagaggctctgcacaaccactacacgcagaagagcctctccctgtctccgggtaaa

SEQ ID NO: 148 - protein, 446 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - DNA, 1338 bp, synthetic construct
  source 1..1338: mol_type = other DNA
gaggtgcagcttcaggagtcaggacctagcctcgtgaaaccttctcagactctgtccctcacatgttctgtcactggcgactccatcaccagtggttactggaactggatccggcaattcccagggaataaacttgagtacatgggtttcataaattacagtggtaacacttactacaatccatctctcagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccttcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 150 - protein, 446 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - DNA, 1338 bp, synthetic construct
  source 1..1338: mol_type = other DNA
gaggtgcagcttcaggagtcaggacctagcctcgtgaaaccttctcagactctgtccctcacatgttctgtcactggcgactccatcaccagtggttactggaactggatccggcaattcccagggaataaacttgagtacatgggtttcataaattacagtggtaacacttactacaatccatctctcagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacttacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtggagcacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 152 - protein, 446 aa, synthetic construct
EVQLQESGPSLVKPSQTLSLTCSVTGDSITSGYWNWIRQFPGNKLEYMGFINYSGNTYYNPSLRSRISITRDTSKNQYFLHLNSVTTEDTATYYCARANWDVFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - DNA, 1338 bp, synthetic construct
  source 1..1338: mol_type = other DNA
gaggtgcagcttcaggagtcaggacctagcctcgtgaaaccttctcagactctgtccctcacatgttctgtcactggcgactccatcaccagtggttactggaactggatccggcaattcccagggaataaacttgagtacatgggtttcataaattacagtggtaacacttactacaatccatctctcagaagtcgaatctccatcactcgagacacatccaagaaccagtacttcctgcacttgaattctgtgactactgaggacacagccacatattactgtgcaagggctaactgggacgtctttgcttactggggccaagggactctggtcactgtctctgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtggagcacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccttcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 154 - protein, 447 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 155 - DNA, 1341 bp, synthetic construct
  source 1..1341: mol_type = other DNA
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggtaccaccgtgacagtttctgccgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgcccccccagtggccgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccaggaaaa

SEQ ID NO: 156 - protein, 447 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMFEALHNHYTQKSLSLSPGK

SEQ ID NO: 157 - DNA, 1341 bp, synthetic construct
  source 1..1341: mol_type = other DNA
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggtaccaccgtgacagtttctgccgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgcccccccagtggccgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccaggaaaa

SEQ ID NO: 158 - protein, 448 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPEFLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 159 - DNA, 1344 bp, synthetic construct
  source 1..1344: mol_type = other DNA
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggtaccaccgtgacagtttctgccgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgctccggagttcctgggcgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccaggaaaa

SEQ ID NO: 160 - protein, 448 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPEFLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMFEALHNHYTQKSLSLSPGK

SEQ ID NO: 161 - DNA, 1344 bp, synthetic construct
  source 1..1344: mol_type = other DNA
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggtaccaccgtgacagtttctgccgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgctccggagttcctgggcgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccaggaaaa

SEQ ID NO: 162 - protein, 448 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPEFLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHGALHNHYTQKSLSLSPGK

SEQ ID NO: 163 - DNA, 1344 bp, synthetic construct
  source 1..1344: mol_type = other DNA
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggtaccaccgtgacagtttctgccgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgctccggagttcctgggcgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacggagcgctgcacaatcattatactcagaaaagcctgagcttgagcccaggaaaa

SEQ ID NO: 164 - protein, 448 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPELLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 165 - DNA, 1344 bp, synthetic construct
  source 1..1344: mol_type = other DNA
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggtaccaccgtgacagtttctgccgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgctccggagctgctgggcgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccaggaaaa

SEQ ID NO: 166 - protein, 448 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPELLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMFEALHNHYTQKSLSLSPGK

SEQ ID NO: 167 - DNA, 1347 bp, synthetic construct
  source 1..1347: mol_type = other DNA
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggtaccaccgtgacagtttctgccgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgctccggagctgctgggcgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccaggaaaatga

SEQ ID NO: 168 - protein, 448 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPELLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHGALHNHYTQKSLSLSPGK

SEQ ID NO: 169 - DNA, 1344 bp, synthetic construct
  source 1..1344: mol_type = other DNA
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggtaccaccgtgacagtttctgccgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgctccggagctgctgggcgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacggagcgctgcacaatcattatactcagaaaagcctgagcttgagcccaggaaaa

SEQ ID NO: 170 - protein, 498 aa, synthetic construct; identical to SEQ ID NO: 38

SEQ ID NO: 171 - DNA, 1494 bp, synthetic construct; identical to SEQ ID NO: 39
  source 1..1494: mol_type = other DNA

SEQ ID NO: 172 - protein, 498 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKLREEQYNSTFRVVSVLTVLHQDWLNGKEYKCKVSNKALPAPIEKTISKTKGQPREPQVYTLPPSREEMTKNQVSLTCLVKGFYPSDIAVEWESNGQPENNYKTTPPVLDSDGSFFLYSKLTVDKSRWQQGNIFSCSVMFEALHNHYTQKSLSLSPGK

SEQ ID NO: 173 - DNA, 1494 bp, synthetic construct
  source 1..1494: mol_type = other DNA
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggtaccaccgtgacagtttctgccgctagcacaaaaggccctagcgtctttccactcgccccatgttctagaagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggcacccagacctatacctgcaatgtcaatcataaacccagtaacacaaaggtagacaagcgggtcgaactgaaaacccccctgggcgacaccacccacacctgtcctagatgccccgagcccaagtcctgcgatacccctccaccttgccccaggtgccctgagcctaagagctgtgataccccccctccctgccctcggtgtccagagcctaagtcttgtgacaccccacccccatgtccaaggtgccccgcccccgaactcctggggggaccttcggtttttctttttccacctaaaccgaaagacaccctgatgatcagcaggacaccagaagtgacatgtgtcgtcgtggacgtatcccatgaggacccggaggtgcagttcaagtggtatgttgatggtgtggaagtgcataatgcaaaaactaagctgcgggaggaacagtacaatagcacattcagagtagttagcgttctgactgtcctgcaccaagattggctgaacgggaaggaatacaagtgtaaggtgagcaacaaagccctgcccgctccaattgagaaaacaatttctaagaccaaaggacagccccgggagccacaggtgtatactctgccgcctagcagggaagagatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacatcttcagttgctcagtgatgttcgaagcgctgcacaatcattatactcagaaaagcctgagcttgagcccgggaaaa

SEQ ID NO: 174 - protein, 448 aa, synthetic construct; identical to SEQ ID NO: 40

SEQ ID NO: 175 - DNA, 1344 bp, synthetic construct; identical to SEQ ID NO: 41
  source 1..1344: mol_type = other DNA

SEQ ID NO: 176 - protein, 448 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMFEALHNHYTQKSLSLSLGK

SEQ ID NO: 177 - DNA, 1344 bp, synthetic construct
  source 1..1344: mol_type = other DNA
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggtaccaccgtgacagtttctgccgctagcacaaaaggccctagcgtctttccactcgccccatgttccagaagcacctctgaaagtactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggcaccaagacctatacatgcaatgtcgaccataaacccagtaacacaaaggtagacaagcgagtcgaaagcaagtacggccccccctgccctagctgccccgcccccgaatttctggggggaccttcggtttttctttttccacctaaaccgaaagacaccctgatgatcagcaggacaccagaagtgacatgtgtcgtcgtggacgtatcccaggaggacccggaggtgcagttcaactggtatgttgatggtgtggaagtgcataatgcaaaaactaagccacgggaggaacagttcaatagcacatacagagtagttagcgttctgactgtcctgcaccaagattggctgaacgggaaggaatacaagtgtaaggtgagcaacaaaggcctgcccagtagcattgagaaaacaatttctaaggccaaaggacagccccgggagccacaggtgtatactctgccgcctagccaagaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcacggctgaccgtggacaaaagcagatggcaggagggaaacgttttcagttgctcagtgatgttcgaagcgctgcacaatcattatactcagaaaagcctgagcttgagcttaggaaaa

SEQ ID NO: 178 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 107 aa, synthetic construct
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 180 - protein, 107 aa, synthetic construct
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 181 - protein, 107 aa, synthetic construct
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 182 - protein, 131 aa, synthetic construct
GNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 183 - protein, 131 aa, synthetic construct; identical to SEQ ID NO: 182

SEQ ID NO: 184 - protein, 445 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 185 - DNA, 1338 bp, synthetic construct
  source 1..1338: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggtacctccgtcacagttagctctgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgcccccccagtggccgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggatga

SEQ ID NO: 186 - protein, 446 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCSVECPPCPAPEFLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 187 - DNA, 1341 bp, synthetic construct
  source 1..1341: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggtacctccgtcacagttagctctgctagcacaaaaggccctagcgtctttccactcgccccaagctcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgcagcgtggagtgccctccttgccccgcccccgaattcctgggcggcccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggatga

SEQ ID NO: 188 - protein, 446 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPEFLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 189 - DNA, 1341 bp, synthetic construct
  source 1..1341: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggtacctccgtcacagttagctctgctagcacaaaaggccctagcgtctttccactcgccccaagctcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaaagctgcgtggagtgccctccttgccccgcccccgaattcctgggcggcccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggatga

SEQ ID NO: 190 - protein, 449 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPEFLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 191 - DNA, 1350 bp, synthetic construct
  source 1..1350: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggtacctccgtcacagttagctctgctagcacaaaaggccctagcgtctttccactcgccccaagctcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaacccaagagctgcgacaagacccacacatgtccaccatgccccgcccccgaattcctgggcggcccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggatga

SEQ ID NO: 192 - protein, 445 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 193 - DNA, 1338 bp, synthetic construct
  source 1..1338: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggtacctccgtcacagttagctctgctagcacaaaaggccctagcgtctttccactcgccccaagctcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaaagctgcgtggagtgccctccttgccccgcccccccagtggccggcccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggatga

SEQ ID NO: 194 - protein, 446 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPEFLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 195 - DNA, 1341 bp, synthetic construct
  source 1..1341: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggtacctccgtcacagttagctctgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgcccccgaattcctgggcggcccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggatga

SEQ ID NO: 196 - protein, 451 aa, synthetic construct
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMFEALHNHYTQKSLSLSPGK

SEQ ID NO: 197 - DNA, 1356 bp, synthetic construct
  source 1..1356: mol_type = other DNA
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggcaccaccgtgacagtttctgccgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggatgaactgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggaaaatga

SEQ ID NO: 198 - protein, 445 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMFEALHNHYTQKSLSLSPG

SEQ ID NO: 199 - DNA, 1338 bp, synthetic construct
  source 1..1338: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggtacctccgtcacagttagctctgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgcccccccagtggccgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggatga

SEQ ID NO: 200 - protein, 446 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCSVECPPCPAPEFLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMFEALHNHYTQKSLSLSPG

SEQ ID NO: 201 - DNA, 1341 bp, synthetic construct
  source 1..1341: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggtacctccgtcacagttagctctgctagcacaaaaggccctagcgtctttccactcgccccaagctcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgcagcgtggagtgccctccttgccccgcccccgaattcctgggcggcccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggatga

SEQ ID NO: 202 - protein, 446 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPEFLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMFEALHNHYTQKSLSLSPG

SEQ ID NO: 203 - DNA, 1341 bp, synthetic construct
  source 1..1341: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggtacctccgtcacagttagctctgctagcacaaaaggccctagcgtctttccactcgccccaagctcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaaagctgcgtggagtgccctccttgccccgcccccgaattcctgggcggcccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggatga

SEQ ID NO: 204 - protein, 449 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPEFLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMFEALHNHYTQKSLSLSPG

SEQ ID NO: 205 - DNA, 1350 bp, synthetic construct
  source 1..1350: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggtacctccgtcacagttagctctgctagcacaaaaggccctagcgtctttccactcgccccaagctcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaacccaagagctgcgacaagacccacacatgtccaccatgccccgcccccgaattcctgggcggcccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggatga

SEQ ID NO: 206 - protein, 446 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPEFLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMFEALHNHYTQKSLSLSPG

SEQ ID NO: 207 - DNA, 1341 bp, synthetic construct
  source 1..1341: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggtacctccgtcacagttagctctgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgcccccgaattcctgggcggcccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggatga

SEQ ID NO: 208 - protein, 450 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - DNA, 1353 bp, synthetic construct
  source 1..1353: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggtacctccgtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaagccgccgggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaatga

SEQ ID NO: 210 - protein, 450 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMFEALHNHYTQKSLSLSPGK

SEQ ID NO: 211 - DNA, 1353 bp, synthetic construct
  source 1..1353: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggtacctccgtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaagccgccgggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggaaaatga

SEQ ID NO: 212 - protein, 450 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVQFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - DNA, 1353 bp, synthetic construct
  source 1..1353: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggtacctccgtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgcagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaatga

SEQ ID NO: 214 - protein, 450 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVQFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMFEALHNHYTQKSLSLSPGK

SEQ ID NO: 215 - DNA, 1353 bp, synthetic construct
  source 1..1353: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggtacctccgtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgcagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggaaaatga

SEQ ID NO: 216 - protein, 450 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSEEDPEVKFNWYVDGVEVHNAKTKPREEQYNSTYRVVSVLTVLHQDWLNGKEYKCKVSNKDLPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMFEALHNHYTQKSLSLSPGK

SEQ ID NO: 217 - DNA, 1353 bp, synthetic construct
  source 1..1353: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggtacctccgtcacagttagctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatccgaggaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagacctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggaaaatga

SEQ ID NO: 218 - protein, 446 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPEAAGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMFEALHNHYTQKSLSLSPG

SEQ ID NO: 219 - DNA, 1341 bp, synthetic construct
  source 1..1341: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggtacctccgtcacagttagctctgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgcccccgaagccgccggcggcccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggatga

SEQ ID NO: 220 - protein, 445 aa, synthetic construct
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGSNSLDYWGQGTSVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVKFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMFEALHNHYTQKSLSLSPG

SEQ ID NO: 221 - DNA, 1338 bp, synthetic construct
  source 1..1338: mol_type = other DNA
caggttcagctggttcagtctggcgccgaggtggccaaacctggcacatctgtgaagctgagctgcaaggccagcggctacaccttcaccgactactggatgcagtgggtcaagcagaggcctggacagggcctcgaatggatcggcacaatctatcctggcgacggcgatacaggctacgcccagaagtttcagggcaaagccacactgaccgccgacaagagcagcaagaccgtgtacatgcacctgagcagcctggcctctgaggatagcgccgtgtactattgcgccagaggcgactactacggcagcaacagcctggattattggggccagggtacctccgtcacagttagctctgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgcccccccagtggccggcccttcggtttttctttttccacctaaaccgaaagacaccctgatgatcagcaggacaccagaagtgacatgtgtcgtcgtggacgtatcccatgaagacccggaggtgaagttcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggatga

SEQ ID NO: 222 - protein, 451 aa, synthetic construct; identical to SEQ ID NO: 196

SEQ ID NO: 223 - DNA, 1353 bp, synthetic construct
  source 1..1353: mol_type = other DNA
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggcaccaccgtgacagtttctgccgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggatgaactgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaggcgctgcacaatcattatactcagaaaagcctgagcttgagcccgggaaaa

SEQ ID NO: 224 - protein, 443 aa, synthetic construct
EVQLVQSGAEVKKPGASVKVSCKASGYTFTSQYMHWVRQAPGQGLEWIGIINPSGVHTSYAQKFQGRVTLTRDTSTSTLYMELSSLRSEDTAVYYCARGSPKGAFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 225 - DNA, 1329 bp, synthetic construct
  source 1..1329: mol_type = other DNA
gaagttcagctggttcagtctggcgccgaagtgaagaaacctggcgcctctgtgaaggtgtcctgcaaggccagcggctacacctttaccagccagtacatgcactgggtccgacaggctccaggacagggactcgagtggatcggcatcatcaatcctagcggcgtgcacaccagctacgcccagaaattccagggcagagtgaccctgaccagagacaccagcaccagcacactgtacatggaactgagcagcctgcggagcgaggataccgccgtgtactattgtgccagaggctctcctaagggcgccttcgattattggggccagggtaccctggtcaccgtgtcctctgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgcccccccagtggccgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccggga

SEQ ID NO: 226 - protein, 443 aa, synthetic construct
EVQLVQSGAEVKKPGASVKVSCKASGYTFTSQYMHWVRQAPGQGLEWIGIINPSGVHTSYAQKFQGRVTLTRDTSTSTLYMELSSLRSEDTAVYYCARGSPKGAFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMFEALHNHYTQKSLSLSPG

SEQ ID NO: 227 - DNA, 1329 bp, synthetic construct
  source 1..1329: mol_type = other DNA
gaagttcagctggttcagtctggcgccgaagtgaagaaacctggcgcctctgtgaaggtgtcctgcaaggccagcggctacacctttaccagccagtacatgcactgggtccgacaggctccaggacagggactcgagtggatcggcatcatcaatcctagcggcgtgcacaccagctacgcccagaaattccagggcagagtgaccctgaccagagacaccagcaccagcacactgtacatggaactgagcagcctgcggagcgaggataccgccgtgtactattgtgccagaggctctcctaagggcgccttcgattattggggccagggtaccctggtcaccgtgtcctctgctagcacaaaaggccctagcgtctttccactcgccccatgttcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgctgtgtggagtgccctccttgccccgcccccccagtggccgggccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccggga

SEQ ID NO: 228 - protein, 444 aa, synthetic construct
EVQLVQSGAEVKKPGASVKVSCKASGYTFTSQYMHWVRQAPGQGLEWIGIINPSGVHTSYAQKFQGRVTLTRDTSTSTLYMELSSLRSEDTAVYYCARGSPKGAFDYWGQGTLVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCSVECPPCPAPEFLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 229 - DNA, 1332 bp, synthetic construct
  source 1..1332: mol_type = other DNA
gaagttcagctggttcagtctggcgccgaagtgaagaaacctggcgcctctgtgaaggtgtcctgcaaggccagcggctacacctttaccagccagtacatgcactgggtccgacaggctccaggacagggactcgagtggatcggcatcatcaatcctagcggcgtgcacaccagctacgcccagaaattccagggcagagtgaccctgaccagagacaccagcaccagcacactgtacatggaactgagcagcctgcggagcgaggataccgccgtgtactattgtgccagaggctctcctaagggcgccttcgattattggggccagggtaccctggtcaccgtgtcctctgctagcacaaaaggccctagcgtctttccactcgccccaagctcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgcagcgtggagtgccctccttgccccgcccccgaattcctgggcggcccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccggga

SEQ ID NO: 230 - protein, 444 aa, synthetic construct
EVQLVQSGAEVKKPGASVKVSCKASGYTFTSQYMHWVRQAPGQGLEWIGIINPSGVHTSYAQKFQGRVTLTRDTSTSTLYMELSSLRSEDTAVYYCARGSPKGAFDYWGQGTLVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCSVECPPCPAPEFLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMFEALHNHYTQKSLSLSPG

SEQ ID NO: 231 - DNA, 1332 bp, synthetic construct
  source 1..1332: mol_type = other DNA
gaagttcagctggttcagtctggcgccgaagtgaagaaacctggcgcctctgtgaaggtgtcctgcaaggccagcggctacacctttaccagccagtacatgcactgggtccgacaggctccaggacagggactcgagtggatcggcatcatcaatcctagcggcgtgcacaccagctacgcccagaaattccagggcagagtgaccctgaccagagacaccagcaccagcacactgtacatggaactgagcagcctgcggagcgaggataccgccgtgtactattgtgccagaggctctcctaagggcgccttcgattattggggccagggtaccctggtcaccgtgtcctctgctagcacaaaaggccctagcgtctttccactcgccccaagctcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaatgcagcgtggagtgccctccttgccccgcccccgaattcctgggcggcccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccggga

SEQ ID NO: 232 - protein, 444 aa, synthetic construct
EVQLVQSGAEVKKPGASVKVSCKASGYTFTSQYMHWVRQAPGQGLEWIGIINPSGVHTSYAQKFQGRVTLTRDTSTSTLYMELSSLRSEDTAVYYCARGSPKGAFDYWGQGTLVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPEFLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 233 - DNA, 1332 bp, synthetic construct
  source 1..1332: mol_type = other DNA
gaagttcagctggttcagtctggcgccgaagtgaagaaacctggcgcctctgtgaaggtgtcctgcaaggccagcggctacacctttaccagccagtacatgcactgggtccgacaggctccaggacagggactcgagtggatcggcatcatcaatcctagcggcgtgcacaccagctacgcccagaaattccagggcagagtgaccctgaccagagacaccagcaccagcacactgtacatggaactgagcagcctgcggagcgaggataccgccgtgtactattgtgccagaggctctcctaagggcgccttcgattattggggccagggtaccctggtcaccgtgtcctctgctagcacaaaaggccctagcgtctttccactcgccccaagctcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaaagctgcgtggagtgccctccttgccccgcccccgaattcctgggcggcccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccggga

SEQ ID NO: 234 - protein, 444 aa, synthetic construct
EVQLVQSGAEVKKPGASVKVSCKASGYTFTSQYMHWVRQAPGQGLEWIGIINPSGVHTSYAQKFQGRVTLTRDTSTSTLYMELSSLRSEDTAVYYCARGSPKGAFDYWGQGTLVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPEFLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMFEALHNHYTQKSLSLSPG

SEQ ID NO: 235 - DNA, 1332 bp, synthetic construct
  source 1..1332: mol_type = other DNA
gaagttcagctggttcagtctggcgccgaagtgaagaaacctggcgcctctgtgaaggtgtcctgcaaggccagcggctacacctttaccagccagtacatgcactgggtccgacaggctccaggacagggactcgagtggatcggcatcatcaatcctagcggcgtgcacaccagctacgcccagaaattccagggcagagtgaccctgaccagagacaccagcaccagcacactgtacatggaactgagcagcctgcggagcgaggataccgccgtgtactattgtgccagaggctctcctaagggcgccttcgattattggggccagggtaccctggtcaccgtgtcctctgctagcacaaaaggccctagcgtctttccactcgccccaagctcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaaagaaaaagctgcgtggagtgccctccttgccccgcccccgaattcctgggcggcccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccggga

SEQ ID NO: 236 - protein, 447 aa, synthetic construct
EVQLVQSGAEVKKPGASVKVSCKASGYTFTSQYMHWVRQAPGQGLEWIGIINPSGVHTSYAQKFQGRVTLTRDTSTSTLYMELSSLRSEDTAVYYCARGSPKGAFDYWGQGTLVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPEFLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 237 - DNA, 1341 bp, synthetic construct
  source 1..1341: mol_type = other DNA
gaagttcagctggttcagtctggcgccgaagtgaagaaacctggcgcctctgtgaaggtgtcctgcaaggccagcggctacacctttaccagccagtacatgcactgggtccgacaggctccaggacagggactcgagtggatcggcatcatcaatcctagcggcgtgcacaccagctacgcccagaaattccagggcagagtgaccctgaccagagacaccagcaccagcacactgtacatggaactgagcagcctgcggagcgaggataccgccgtgtactattgtgccagaggctctcctaagggcgccttcgattattggggccagggtaccctggtcaccgtgtcctctgctagcacaaaaggccctagcgtctttccactcgccccaagctcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaacccaagagctgcgacaagacccacacatgtccaccatgccccgcccccgaattcctgggcggcccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccggga

SEQ ID NO: 238 - protein, 447 aa, synthetic construct
EVQLVQSGAEVKKPGASVKVSCKASGYTFTSQYMHWVRQAPGQGLEWIGIINPSGVHTSYAQKFQGRVTLTRDTSTSTLYMELSSLRSEDTAVYYCARGSPKGAFDYWGQGTLVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPEFLGGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMFEALHNHYTQKSLSLSPG

SEQ ID NO: 239 - DNA, 1341 bp, synthetic construct
  source 1..1341: mol_type = other DNA
gaagttcagctggttcagtctggcgccgaagtgaagaaacctggcgcctctgtgaaggtgtcctgcaaggccagcggctacacctttaccagccagtacatgcactgggtccgacaggctccaggacagggactcgagtggatcggcatcatcaatcctagcggcgtgcacaccagctacgcccagaaattccagggcagagtgaccctgaccagagacaccagcaccagcacactgtacatggaactgagcagcctgcggagcgaggataccgccgtgtactattgtgccagaggctctcctaagggcgccttcgattattggggccagggtaccctggtcaccgtgtcctctgctagcacaaaaggccctagcgtctttccactcgccccaagctcaagaagcaccagcgagtcaactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcaattttggcacccagacctatacatgcaatgtcgatcataaacccagtaacacaaaggtagacaagacagtcgaacccaagagctgcgacaagacccacacatgtccaccatgccccgcccccgaattcctgggcggcccttcggtttttctttttccacctaaaccgaaagacaccctgatgatcagcaggacaccagaagtgacatgtgtcgtcgtggacgtatcccatgaagacccggaggtgcaattcaactggtatgttgatggtgtggaagtgcataatgcaaaaactaagccacgggaggaacagttcaatagcacattcagagtagttagcgttctgactgtcgtgcaccaagattggctgaacgggaaggaatacaagtgtaaggtgagcaacaaaggtctgcccgctccaattgagaaaacaatttctaagaccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccatgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccggga

SEQ ID NO: 240 - protein, 9 aa, synthetic construct
PKSCDKTHT

SEQ ID NO: 241 - protein, 6 aa, synthetic construct
RKCCVE

SEQ ID NO: 242 - protein, 10 aa, synthetic construct
EPKSCDKTHT

SEQ ID NO: 243 - protein, 7 aa, synthetic construct
ERKCCVE

SEQ ID NO: 244 - protein, 12 aa, synthetic construct
ELKTPLGDTTHT

SEQ ID NO: 245 - protein, 7 aa, synthetic construct
ESKYGPP

SEQ ID NO: 246 - protein, 5 aa, synthetic construct
CPPCP

SEQ ID NO: 247 - protein, 5 aa, synthetic construct
CPRCP

SEQ ID NO: 248 - protein, 5 aa, synthetic construct
CPSCP

SEQ ID NO: 249 - protein, 7 aa, synthetic construct
APELLGG

SEQ ID NO: 250 - protein, 6 aa, synthetic construct
APPVAG

SEQ ID NO: 251 - protein, 7 aa, synthetic construct
APEFLGG

SEQ ID NO: 252 - protein, 4 aa, synthetic construct
EFEG

SEQ ID NO: 253 - protein, 4 aa, synthetic construct
EFLG

SEQ ID NO: 254 - protein, 4 aa, synthetic construct
ELLG

SEQ ID NO: 255 - protein, 4 aa, synthetic construct
PVAG

SEQ ID NO: 256 - protein, 5 aa, synthetic construct
EFLGG

SEQ ID NO: 257 - protein, 5 aa, synthetic construct
ELLGG

SEQ ID NO: 258 - protein, 5 aa, synthetic construct
EAAGG

SEQ ID NO: 259 - protein, 4 aa, synthetic construct
FLGG

SEQ ID NO: 260 - protein, 4 aa, synthetic construct
LLGG